(19) United States
(12) Patent Application Publication
(10) Pub. No.: US 2026/0185088 A1
(43) Pub. Date: Jul. 2, 2026

(54) Modified Short Interfering Nucleic Acid (siNA) Molecules and Uses Thereof

(71) Applicant: Aligos Therapeutics, Inc., South San Francisco, CA (US)

(72) Inventors: Leonid Beigelman, San Mateo, CA (US); Vivek Kumar Rajwanshi, Cupertino, CA (US); Jin Hong, Pacifica, CA (US); Kellan Passow, South San Francisco, CA (US); Saul Martinez Montero, San Bruno, CA (US); Santhosh Kumar Thatikonda, Fremont, CA (US); David B. Smith, South San Francisco, CA (US)

(73) Assignee: Aligos Therapeutics, Inc., South San Francisco, CA (US)

(21) Appl. No.: 19/126,545

(22) PCT Filed: Oct. 30, 2023
(86) PCT No.: PCT/US2023/078224

§ 371 Date: May 1, 2025

Related U.S. Application Data
(60) Provisional application No. 63/421,946, filed on Nov. 2, 2022.
(60) Provisional application No. 63/591,984, filed on Oct. 20, 2023.

(51) Int. Cl. C12N 15/113

(52) U.S. Cl. CPC C12N 15/113; C12N 15/1131; C12N 2310/14; C12N 2310/3231; C12N 2310/3341; C12N 2310/351

(57) ABSTRACT

Described are short interfering nucleic acid (siNA) molecules comprising modified nucleotides, compositions, and methods and uses thereof.

### CROSS-REFERENCE TO RELATED APPLICATION

This application claims priority under 35 U.S.C. § 119 to Provisional Application Ser. No. 63/421,946, filed Nov. 2, 2022, and to Provisional Application Ser. No. 63/591,984, filed Oct. 20, 2023, the disclosures of which are incorporated herein by reference in their entireties.

### TECHNICAL FIELD

Described are short interfering nucleic acid (siNA) molecules comprising modified nucleotides, compositions, and methods and uses thereof.

### BACKGROUND

RNA interference (RNAi) is a biological response to double-stranded RNA that mediates resistance to both endogenous parasitic and exogenous pathogenic nucleic acids, and regulates the expression of protein-coding genes. The short interfering nucleic acids (siNA), such as siRNA, have been developed for RNAi therapy to treat a variety of diseases. For instance, RNAi therapy has been proposed for the treatment of metabolic diseases, neurodegenerative diseases, cancer, and pathogenic infections (See e.g., Rondindone, Biotechniques, 2018, 40 (4S), doi.org/10.2144/000112163, Boudreau and Davidson, Curr Top Dev Biol, 2006, 75:73-92, Chalbatani et al., Int J Nanomedicine, 2019, 14:3111-3128, Arbuthnot, Drug News Perspect, 2010, 23 (6): 341-50, and Chernikov et. al., Front. Pharmacol., 2019, doi.org/10.3389/fphar.2019.00444, each of which are incorporated by reference in their entirety). However, major limitations of RNAi therapy are the ability to effectively deliver siRNA to target cells and the degradation of the siRNA.

The present disclosure improves the delivery and stability of siNA molecules by providing siNA molecules comprising modified nucleobases. The siNA molecules of the present disclosure provide optimized combinations and numbers of modified nucleotides, nucleotide lengths, design (e.g., blunt ends or overhangs, internucleoside linkages, conjugates), and modification patterns for improving the delivery and stability of siNA molecules.

### SUMMARY

Described herein are short interfering nucleic acid (siNA) molecules comprising novel modified nucleobase monomers, phosphate mimics, and/or other modifications. Also described herein are methods of using the disclosed siNA molecules for treating various diseases and conditions.

In a first aspect, the present disclosure provides an oligonucleotide comprising a nucleotide comprising a structure selected from:

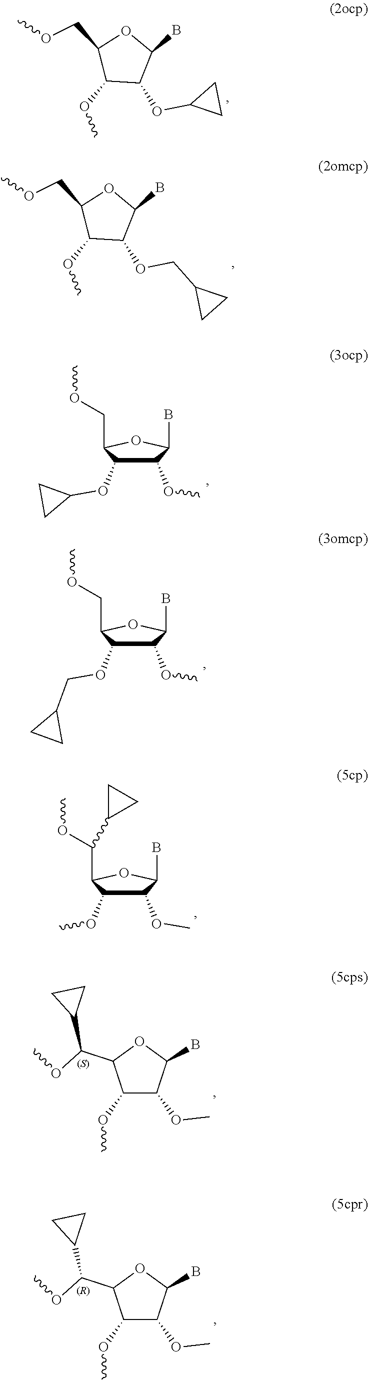

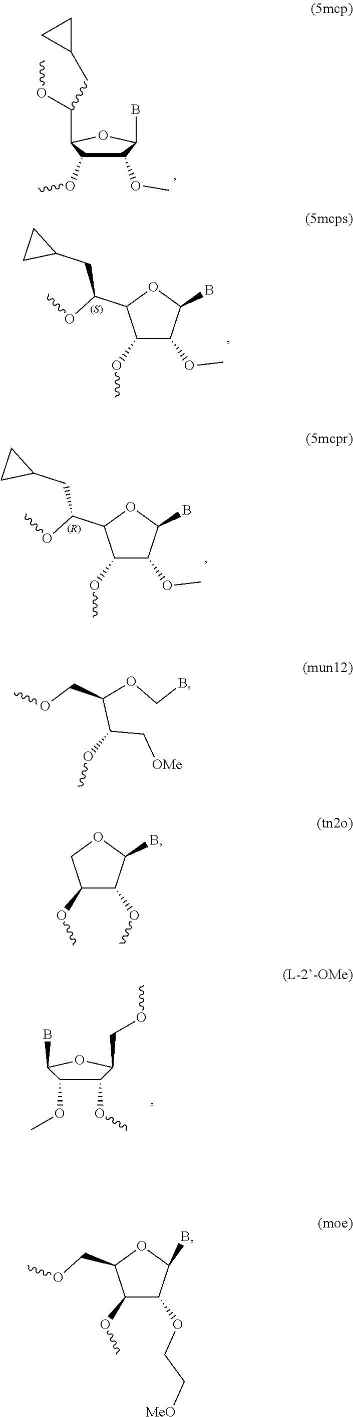

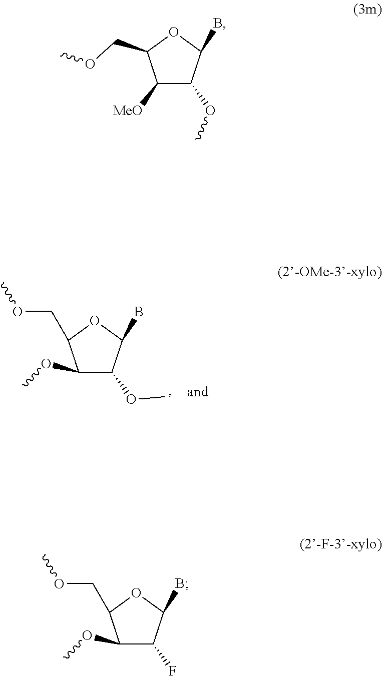

wherein B is a nucleobase selected from adenine, guanine, cytosine, thymine, and uracil, aryl, heteroaryl, or H, and wherein [symbol] represents a phosphodiester linkage, a phosphorothioate linkage, a mesyl phosphoroamidate linkage, or H. For example, the oligonucleotide comprises a nucleotide comprising a structure selected from:

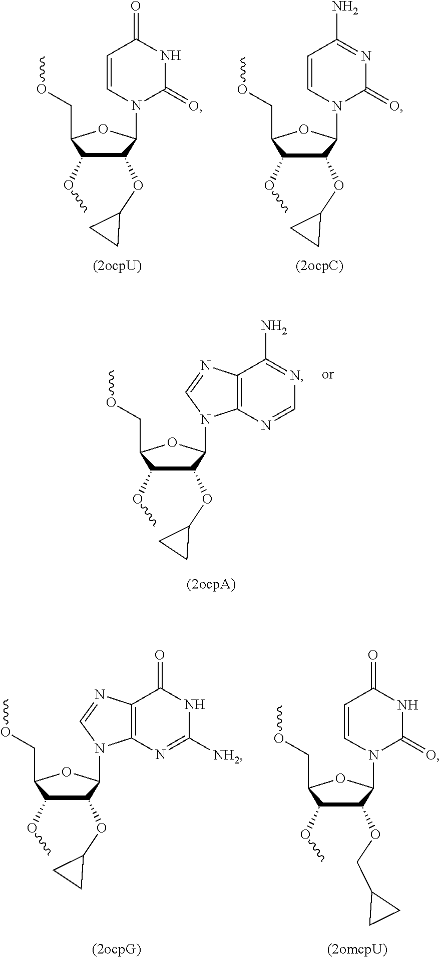

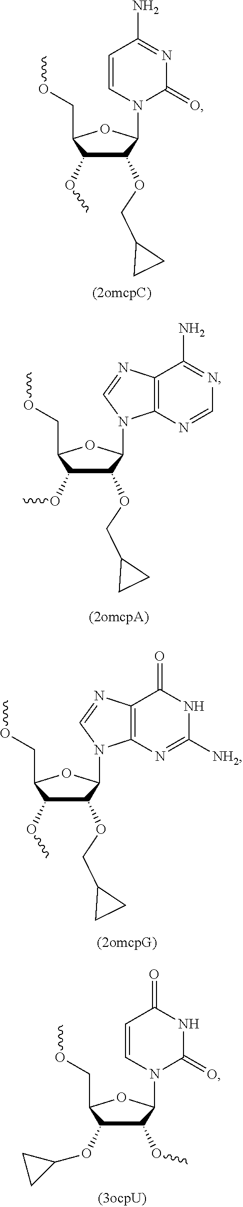

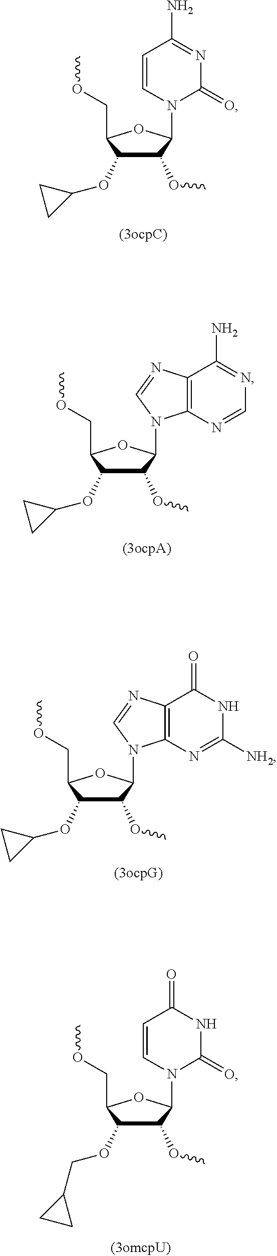

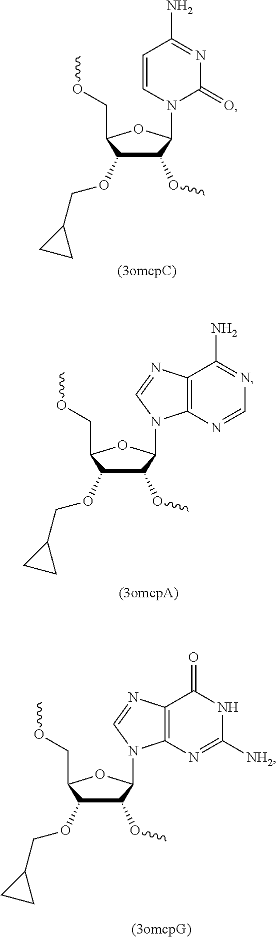

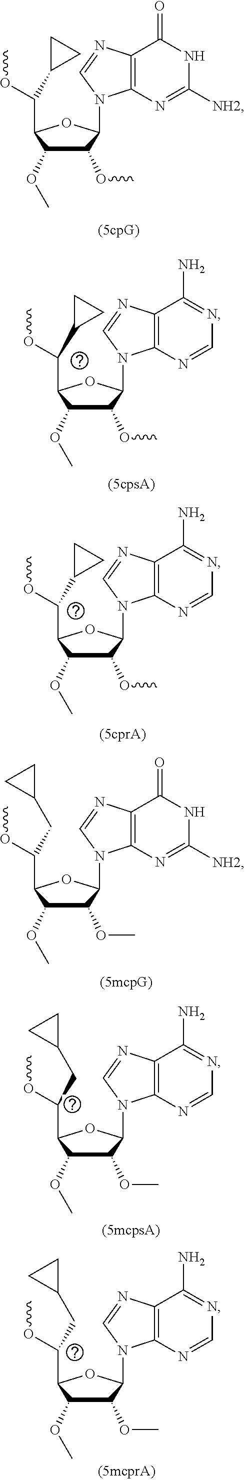

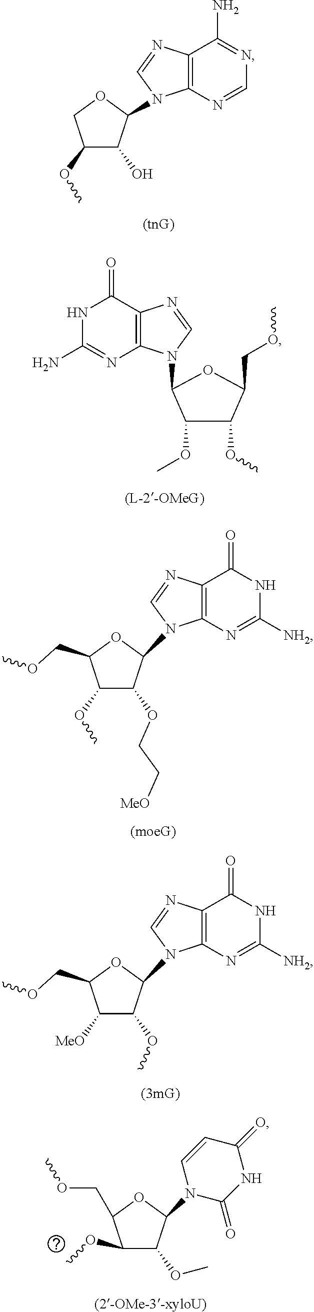

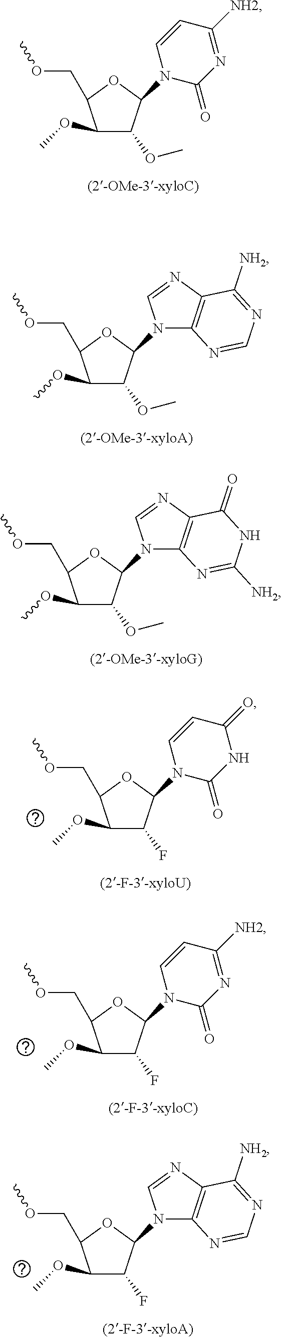

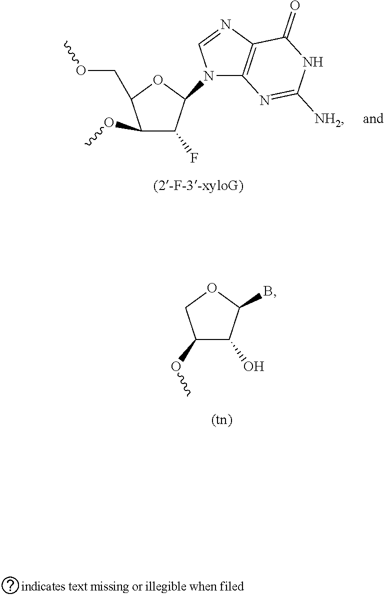

wherein B is a nucleobase, aryl, heteroaryl, or H, and wherein [symbol] represents a phosphodiester linkage, a phosphorothioate linkage, a mesyl phosphoroamidate linkage, or H.

In some embodiments, the oligonucleotide comprises at least 2, at least 3, at least 4, or at least 5 nucleotides comprising a structure independently selected from:

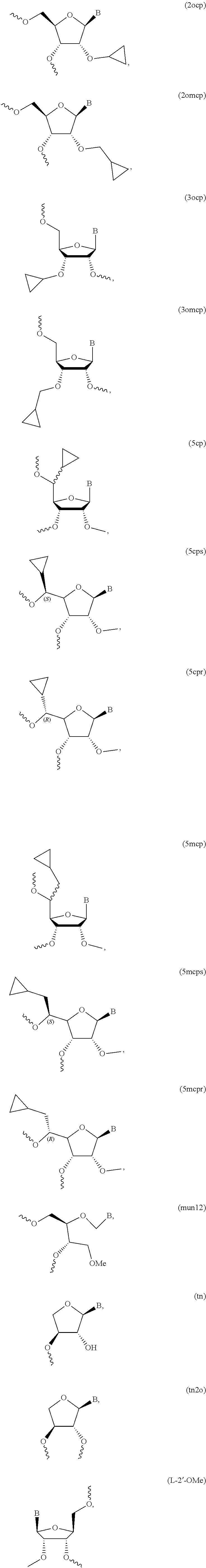

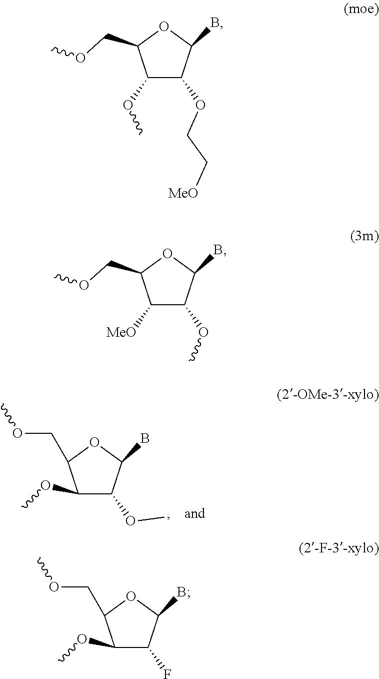

wherein B is a nucleobase, aryl, heteroaryl, or H, and wherein [symbol] represents a phosphodiester linkage, a phosphorothioate linkage, a mesyl phosphoroamidate linkage, or H.

In a second aspect, the present disclosure provides an oligonucleotide comprising a nucleotide analog comprising a structure of:

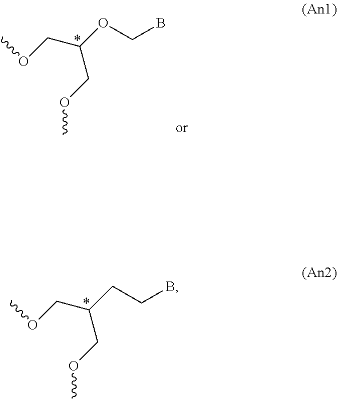

wherein B is a nucleobase, an aryl, heteroaryl, or H; wherein [symbol] represents a phosphodiester linkage, a phosphorothioate linkage, or H; and wherein * represent chiral center (e.g., R or S isomer). For example, oligonucleotide comprises at least 2, at least 3, at least 4, or at least 5 nucleotide analogs comprising a structure independently selected from:

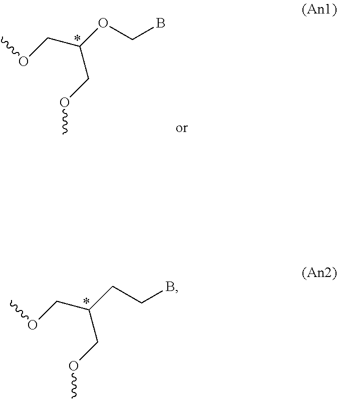

wherein B is a nucleobase, an aryl, heteroaryl, or H; wherein [symbol] represents a phosphodiester linkage, a phosphorothioate linkage, or H; and wherein * represent chiral center (e.g., R or S isomer).

In a third aspect, the present disclosure provides an oligonucleotide comprising a structure selected from:

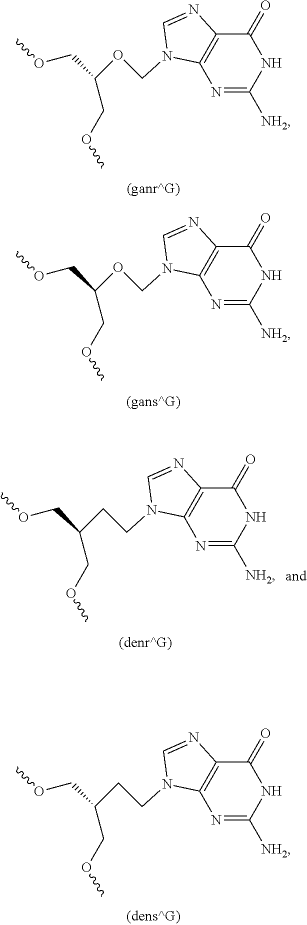

wherein B is a nucleobase, aryl, heteroaryl, or H; wherein [symbol] represents a phosphodiester linkage, a phosphorothioate linkage, or a mesyl phosphoroamidate linkage.

In a fourth aspect, the present disclosure provides an oligonucleotide comprising a structure of.

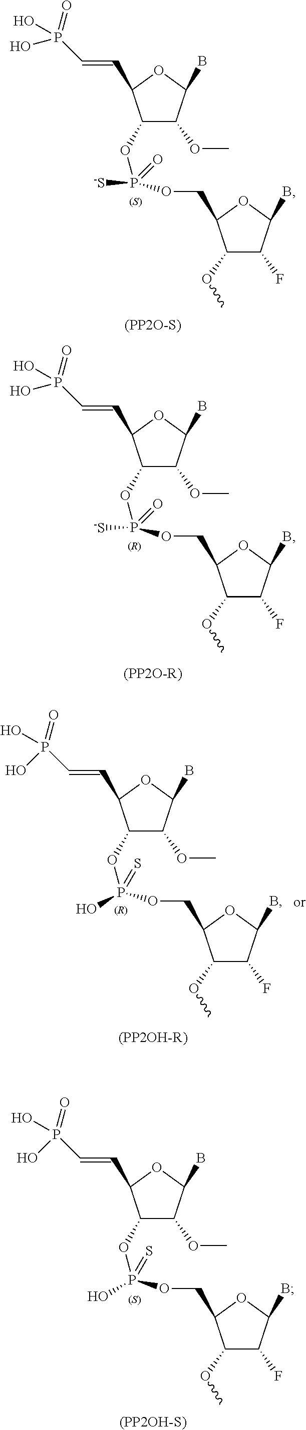

wherein each B is undependably selected from a nucleobase, aryl, heteroaryl, and H; wherein [symbol] represents a phosphodiester linkage, a phosphorothioate linkage, or a mesyl phosphoroamidate linkage.

In some embodiments, the oligonucleotide is selected from a short interfering nucleic acid (siNA), an antisense oligonucleotide (ASO), a steric blocker, a short hairpin RNA (shRNA), and an mRNA.

In a fifth aspect, the present disclosure provides a short interfering nucleic acid (siNA), comprising a sense strand and an antisense strand, wherein the sense strand, the antisense strand, or both comprise at least 1, at least 2, at least 3, at least 4, or at least 5 nucleotide(s) independently selected from:

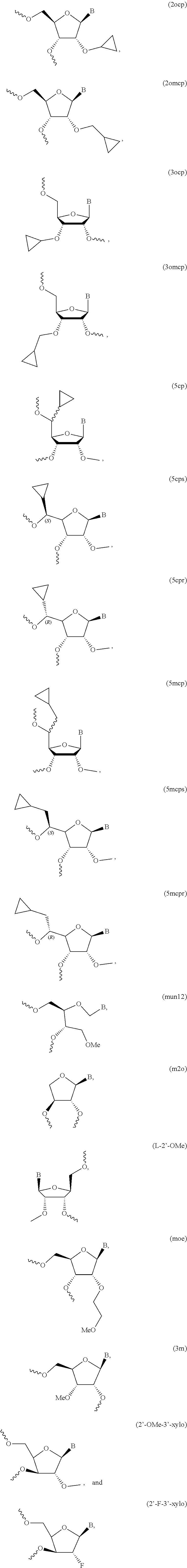

or at least 1, at least 2, at least 3, at least 4, or at least 5 nucleotide analog(s) independently selected from:

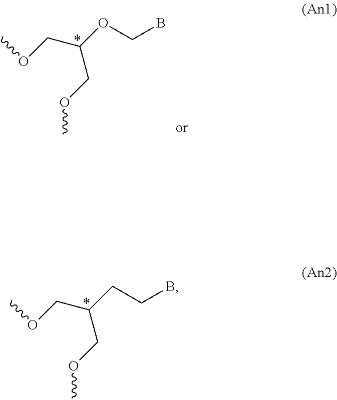

wherein B is a nucleobase, an aryl, heteroaryl, or H; wherein [symbol] represents a phosphodiester linkage, a phosphorothioate linkage, or H; and wherein * represent chiral center (e.g., R or S isomer).

In a sixth aspect, the present disclosure provides a short interfering nucleic acid (siNA) comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide, and
   an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 7, 8, 10, 14, 16, 17, and/or 18 from the 5′ end of the second nucleotide sequence is a 2′-fluoro nucleotide;
      wherein the sense strand and/or the antisense strand comprise at least 1, at least 2, at least 3, at least 4, or at least 5 nucleotide(s) selected from:

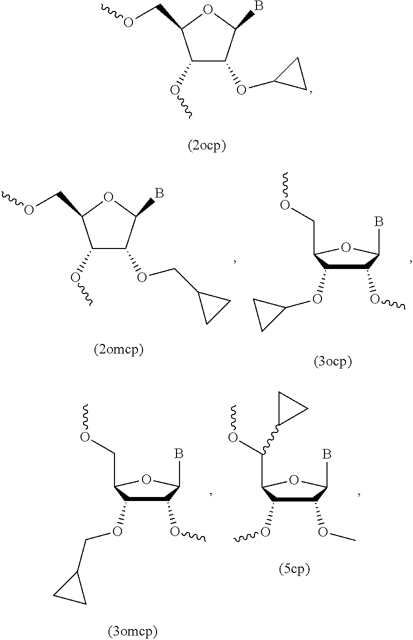

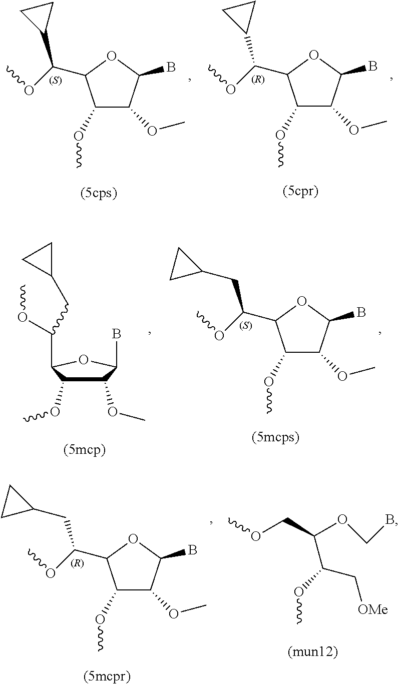

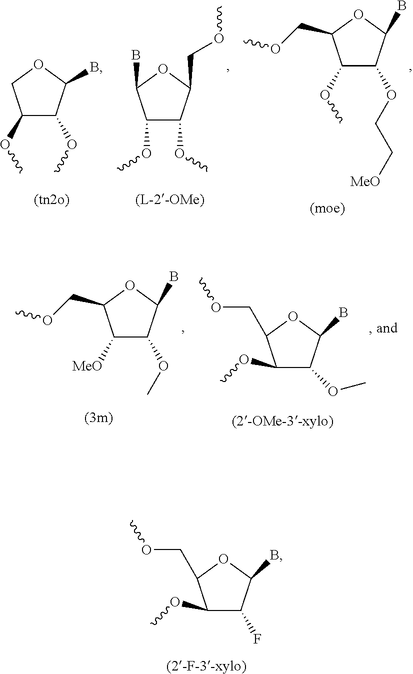

or at least 1, at least 2, at least 3, at least 4, or at least 5 nucleotide analog(s) independently selected from:

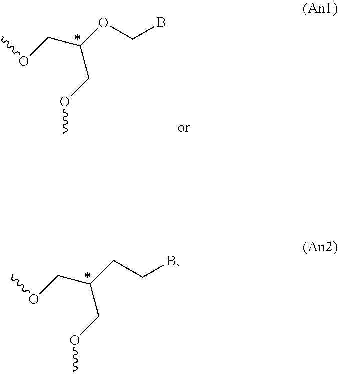

wherein B is a nucleobase, an aryl, heteroaryl, or H; wherein [symbol] represents a phosphodiester linkage, a phosphorothioate linkage, or H; and wherein * represent chiral center (e.g., R or S isomer).

In some embodiments, the antisense strand of the siRNA comprises a 5′-stabilized end cap selected from:

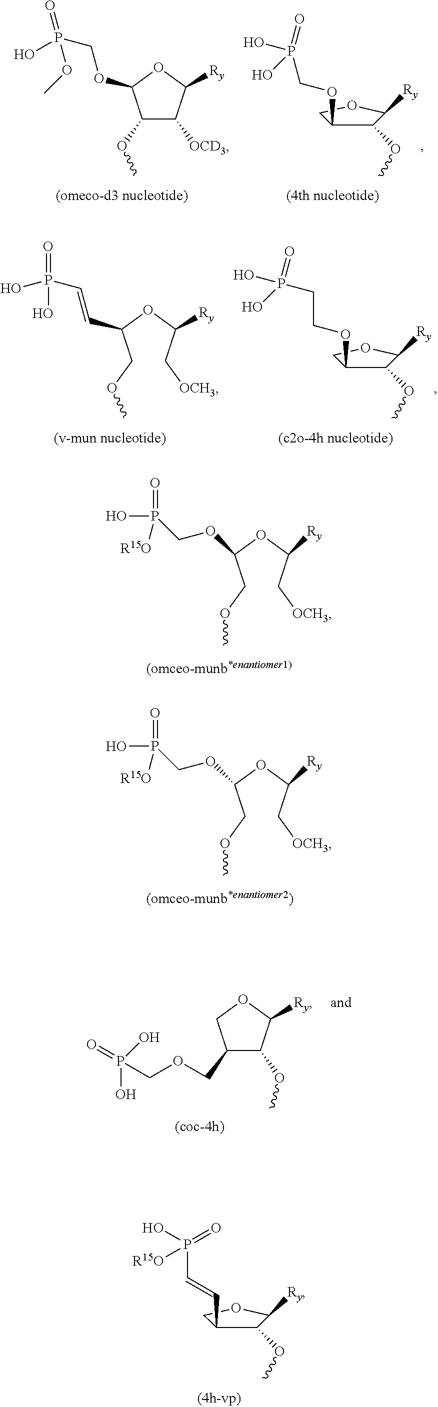

wherein R_{y} is a nucleobase and R¹⁵ is H or CH₃, and wherein [symbol] represents a phosphodiester linkage, a phosphorothioate linkage, or a mesyl phosphoroamidate linkage.

In some embodiments, the antisense strand of the siRNA comprises a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61), Formula (62), and Formula (63), wherein Rₓ is a nucleobase, aryl, heteroaryl, or H.

In some embodiments, the antisense strand of the siRNA comprises a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′, wherein Rₓ is a nucleobase, aryl, heteroaryl, or H.

In some embodiments, the antisense strand of the siRNA comprises a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY).

In some embodiments, the antisense strand of the siRNA comprises a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY).

In some embodiments, the antisense strand of the siRNA comprises a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′).

In a seventh aspect, the present disclosure provides a short interfering nucleic acid (siNA), comprising a sense strand and an antisense strand, wherein the antisense strand comprises a 5′vinyl phosphonate dimer moiety comprising a structure of:

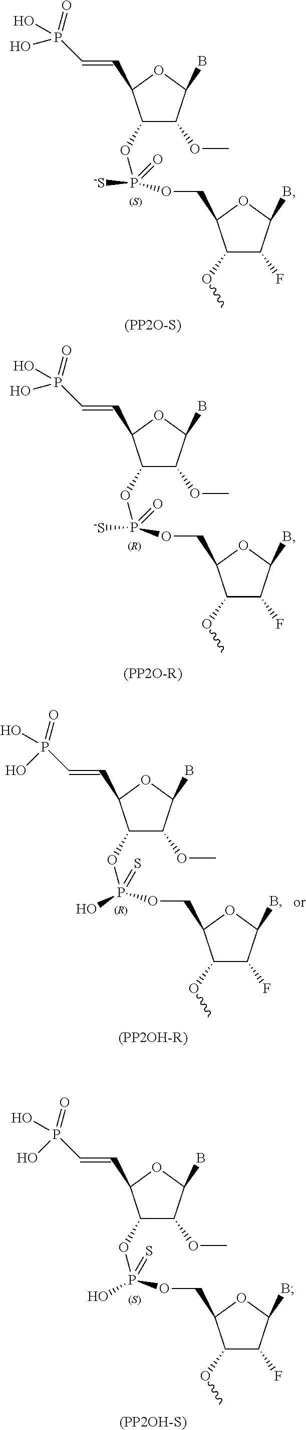

wherein each B is independently selected from a nucleobase, aryl, heteroaryl, and H; wherein [symbol] represents a phosphodiester linkage, a phosphorothioate linkage, or a mesyl phosphoroamidate linkage.

In some embodiments, the sense strand of the siNA, the antisense strand or the siNA, or both comprise at least 1, at least 2, at least 3, at least 4, or at least 5 nucleotide(s) comprising a structure independently selected from:

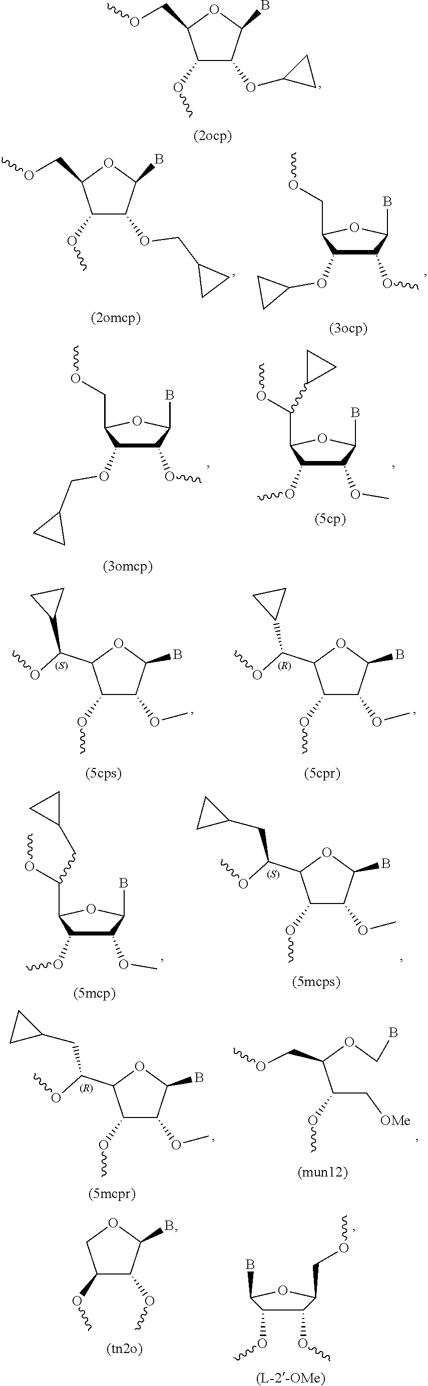

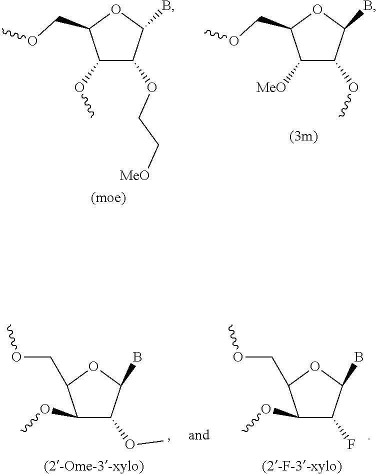

or at least 1, at least 2, at least 3, at least 4, or at least 5 nucleotide analog(s) comprising a structure independently selected from:

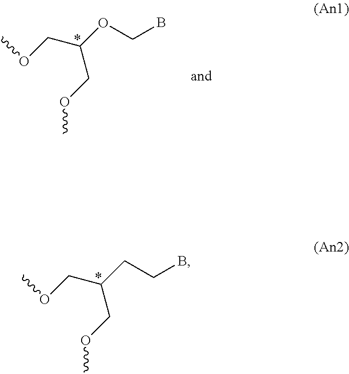

wherein B is a nucleobase, an aryl, heteroaryl, or H; wherein [symbol] represents a phosphodiester linkage, a phosphorothioate linkage, or H; and wherein * represent chiral center (e.g., R or S isomer). In some embodiments, the sense strand, the antisense strand, or both each independently comprise 1 or more phosphorothioate internucleoside linkages. In some embodiments, the siNA further comprises a phosphorylation blocker.

In some embodiments, the sense strand of the siNA comprises at least 1, 2, 3, 4, 5, 6, 7, 8, 9, 10, 11, 12, 13, 14, 15 or more phosphorothioate internucleoside linkages. In some embodiments, at least one phosphorothioate internucleoside linkage in the sense strand is between the nucleotides at positions 1 and 2 from the 5′ end of the sense strand, and/or at least one phosphorothioate internucleoside linkage in the sense strand is between the nucleotides at positions 2 and 3 from the 5′ end of the sense strand.

In some embodiments, the antisense strand of the siNA further comprises at least 1, 2, 3, 4, 5, 6, 7, 8, 9, 10, 11, 12, 13, 14, 15 or more phosphorothioate internucleoside linkages. In some embodiments, at least one phosphorothioate internucleoside linkage in the antisense strand is between the nucleotides at positions 1 and 2 from the 5′ end of the antisense strand at least one phosphorothioate internucleoside linkage in the antisense strand is between the nucleotides at positions 2 and 3 from the 5′ end of the antisense strand, at least one phosphorothioate internucleoside linkage in the antisense strand is between the nucleotides at positions 1 and 2 from the 3′ end of the secant sense strand, and/or at least one phosphorothioate internucleoside linkage is between the nucleotides at positions 2 and 3 from the 3′ end of the antisense strand.

In some embodiments, the sense strand of the siNA comprises at least 1, 2, 3, 4, 5, 6, 7, 8, 9, 10, 11, 12, 13, 14, 15 or more mesyl phosphoroamidate internucleoside linkages. In some embodiments, at least one mesyl phosphoroamidate internucleoside linkage in the sense strand is between the nucleotides at positions 1 and 2 from the 5′ end of the sense strand, and/or at least one mesyl phosphoroamidate internucleoside linkage is between the nucleotides at positions 2 and 3 from the 5′ end of the sense strand.

In some embodiments, the antisense strand of the siNA further comprises at least 1, 2, 3, 4, 5, 6, 7, 8, 9, 10, 11, 12, 13, 14, 15 or more mesyl phosphoroamidate internucleoside linkages. In some embodiments, at least one mesyl phosphoroamidate internucleoside linkage in the antisense strand is between the nucleotides at positions 1 and 2 from the 5′ end of the antisense strand, at least one mesyl phosphoroamidate internucleoside linkage in the antisense strand is between the nucleotides at positions 2 and 3 from the 5′ end of the antisense strand, at least one mesyl phosphoroamidate internucleoside linkage in the antisense strand is between the nucleotides at positions 1 and 2 from the 3′ end of the antisense strand, and/or at least one mesyl phosphoroamidate internucleoside linkage is between the nucleotides at positions 2 and 3 from the 3′ end of the antisense strand.

In some embodiments, the sense strand of the siNA, the antisense strand of the siNA, or both each independently comprise at least 1, at least 2, at least 3, at least 4, or at least 5 or more of

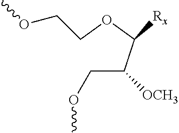

wherein Rₓ is a nucleobase, aryl, heteroaryl, or H,

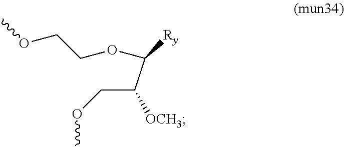

wherein R_{y} is a nucleobase,

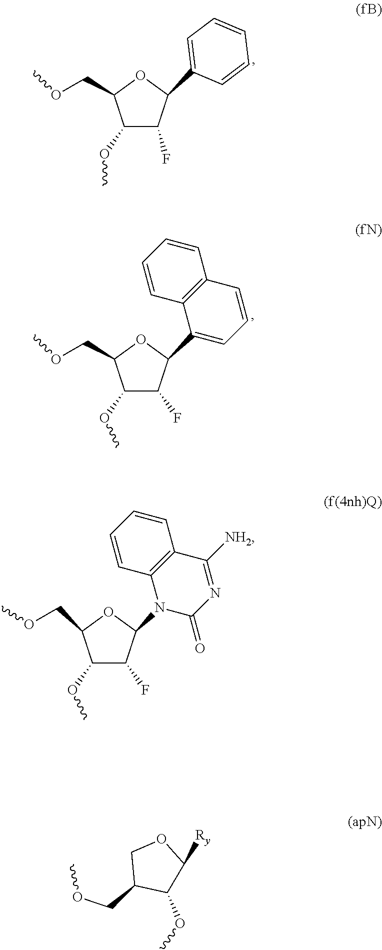

wherein R_{y} is a nucleobase, or combinations thereof.

In some embodiments, the siNA further comprises a galactosamine. In some embodiments, the galactosamine is N-acetylgalactosamine (GalNAc) of Formula (VI):

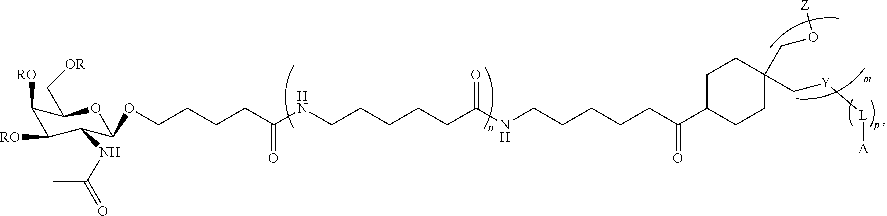

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide. In some embodiments, the galactosamine is N-acetylgalactosamine (GalNAc) of Formula (VII):

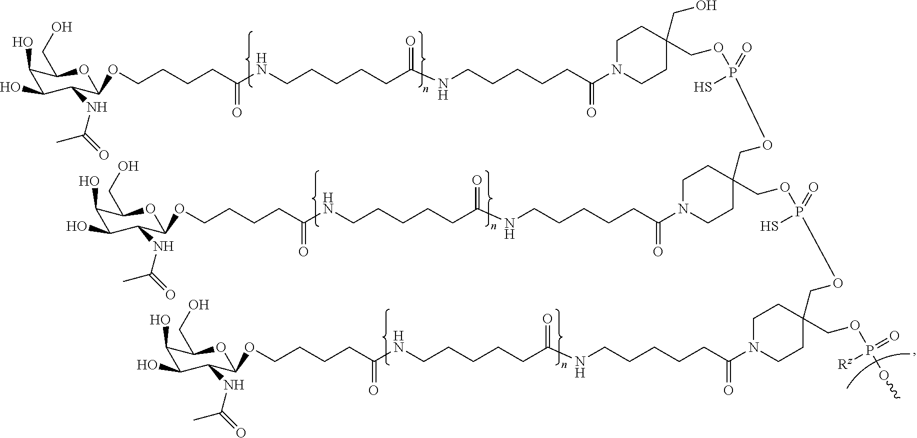

wherein R^{z} is OH or SH; and each n is independently 1 or 2.

In some embodiments, at least one end of the siNA is a blunt end, at least one end of the siNA comprises an overhang, wherein the overhang comprises at least one nucleotide, or both ends of the siNA comprise an overhang, wherein the overhang comprises at least one nucleotide.

In some embodiments, target gene of the siNA is a viral gene, a gene is from a DNA virus, a gene from a double-stranded DNA (dsDNA) virus, a gene from a hepadnavirus, a gene from a hepatitis B virus (HBV), a gene from a HBV of any one of genotypes A-J, or the target gene is selected from the S gene or X gene of a HBV.

In a seventh aspect, the present disclosure provides an siNA as shown in Table 1, Table 2, Table 3, Table 4, Table 5, Table 6, Table 7, Table 8, Table 9, Table 10, Table 11, Table 12, Table 13, Table 14, Table 15, Table 16. Table 17, or Table 18.

The present disclosure provides composition comprising an siNA according to any one of the siNAs disclosed herein, and a pharmaceutically acceptable excipient. In some embodiments, the composition comprises 2, 3, 4, 5, 6, 7, 8, 9, 10 or more of any of the siNAs disclosed herein. In some embodiments, the composition comprises an additional treatment agent. In some embodiments, the additional treatment agent is selected from a nucleotide analog, nucleoside analog, a capsid assembly modulator (CAM), a recombinant interferon, an entry inhibitor, a small molecule immunomodulatory, and oligonucleotide therapy. In some embodiments, the oligonucleotide therapy is an additional siNA, an antisense oligonucleotide (ASO), NAPs, or STOPS™.

The present disclosure provides methods of treating a disease in a subject in need thereof, comprising administering to the subject the siNA disclosed herein or a composition comprising the siNA disclosed herein. The present disclosure further provides uses of the disclosed siNA and compositions for treating a disease in a subject. The present disclosure further provides siNA and compositions for use in treating a disease in a subject.

In some embodiments of the disclosed methods and uses, the disease is a viral disease, which is optionally caused by a DNA virus or a double stranded DNA (dsDNA) virus. In some embodiments, the dsDNA virus is a hepadnavirus. In some embodiments, the hepadnavirus is a hepatitis B virus (HBV), and optionally wherein the HBV is selected from HBV genotypes A-J. In some embodiments, the methods and uses may further comprise administering an additional HBV treatment agent. In some embodiments, the siNA or the composition and the additional HBV treatment agent are administered concurrently or administered sequentially. In some embodiments, the additional HBV treatment agent is selected from a nucleotide analog, nucleoside analog, a capsid assembly modulator (CAM), a recombinant interferon, an entry inhibitor, a small molecule immunomodulator and oligonucleotide therapy. In some embodiments, the viral disease is a disease caused by a coronavirus, and optionally wherein the coronavirus is SARS-COV-2.

In some embodiments of the disclosed methods and uses, the disease is a liver disease. In some embodiments, the liver disease is a nonalcoholic fatty liver disease (NAFLD) or hepatocellular carcinoma (HCC). In some embodiments, the NAFLD is nonalcoholic steatohepatitis (NASH). Some embodiments may further comprise administering to the subject a liver disease treatment agent. In some embodiments, the liver disease treatment agent is selected from a peroxisome proliferator-activator receptor (PPAR) agonist, farnesoid X receptor (FXR) agonist, lipid-altering agent, and incretin-based therapy. In some embodiments, (i) the PPAR agonist is selected from a PPARα agonist, dual PPARα/δ agonist, PPARγ agonist, and dual PPARα/γ agonist; (ii) the lipid-altering agent is aramchol; or (iii) the incretin-based therapy is a glucagon-like peptide 1 (GLP-1) receptor agonist or dipeptidyl peptidase 4 (DPP-4) inhibitor. In some embodiments, the siNA or composition and the liver disease treatment agent are administered concurrently or administered sequentially.

In some embodiments of the disclosed methods and uses, the siNA or the composition is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg.

In some embodiments of the disclosed methods and uses, the siNA or the composition is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

In some embodiments of the disclosed methods and uses, the siNA or the composition is administered at least 1, 2, 3, 4, 5, 6, 7, 8, 9, or 10 times.

In some embodiments of the disclosed methods and uses, the siNA or the composition is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month.

In some embodiments of the disclosed methods and uses, the siNA or the composition is administered at least once every 1, 2, 3, 4, 5, 6, 7, 8, 9, 10, 11, 12, 13, 14, 15, 16, 17, 18, 19, 20, or 21 days.

In some embodiments of the disclosed methods and uses, the siNA or the composition is administered for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

In some embodiments of the disclosed methods and uses, the siNA or the composition is administered at a single dose of 5 mg/kg or 10 mg/kg, at three doses of 10 mg/kg once a week, at three doses of 10 mg/kg once every three days, or at five doses of 10 mg/kg once every three days.

In some embodiments of the disclosed methods and uses, the siNA or the composition is administered at six doses of ranging from 1 mg/kg to 15 mg/kg, 1 mg/kg to 10 mg/kg, 2 mg/kg to 15 mg/kg, 2 mg/kg to 10 mg/kg, 3 mg/kg to 15 mg/kg, or 3 mg/kg to 10 mg/kg; wherein the first dose and second dose are optionally administered at least 3 days apart; wherein the second dose and third dose are optionally administered at least 4 days apart; and wherein the third dose and fourth dose, fourth dose and fifth dose, and or fifth dose and sixth dose are optionally administered at least 7 days apart.

In some embodiments of the disclosed methods and uses, the siNA or the composition are administered in a particle or viral vector, wherein the viral vector is optionally selected from a vector of adenovirus, adeno-associated virus (AAV), alphavirus, flavivirus, herpes simplex virus, lentivirus, measles virus, picornavirus, poxvirus, retrovirus, and rhabdovirus. In some embodiments, the viral vector is a recombinant viral vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

In some embodiments of the disclosed methods and uses, the siNA or the composition is administered systemically or administered locally.

In some embodiments of the disclosed methods and uses, the siNA or the composition is administered intravenously, subcutaneously, or intramuscularly.

In a eighth aspect, the present disclosure provides an siNA comprising a sense strand and an antisense strand, wherein the antisense strand comprises a 3′ overhang comprising at least one modified nucleotide selected from the structure of:

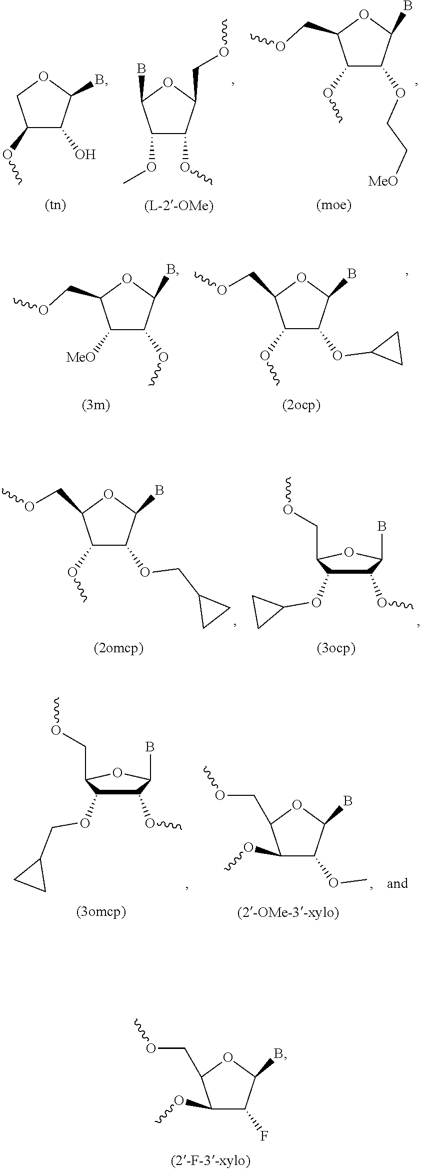

wherein B is a nucleobase, aryl, heteroaryl, or H, and wherein [symbol] represents a phosphodiester linkage, a phosphorothioate linkage, a mesyl phosphoroamidate linkage, or H. For example, the nucleotide comprises a structure selected from:

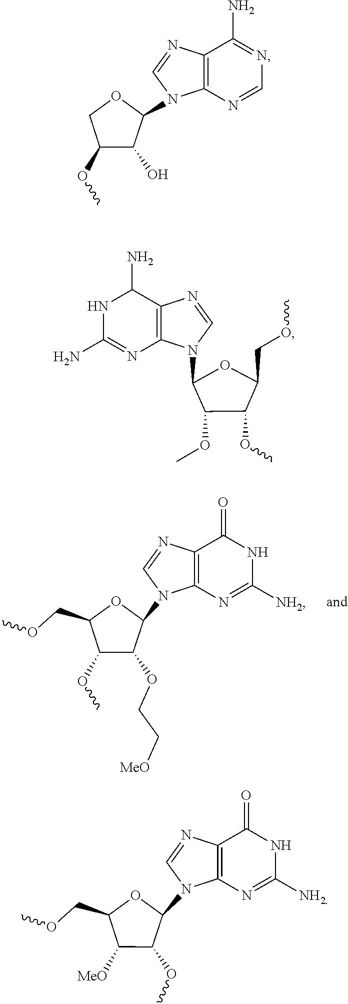

In some embodiments, the modified nucleotide is the last nucleotide or second to last nucleotide at the 3′ end of the antisense strand. In some embodiments, the siNA is resistant to nuclease activity relative to a siNA of the same sequence without the modified nucleotide in the 3′ overhang.

In a seventh aspect, the present disclosure provides a phosphoramidite comprising a structure of:

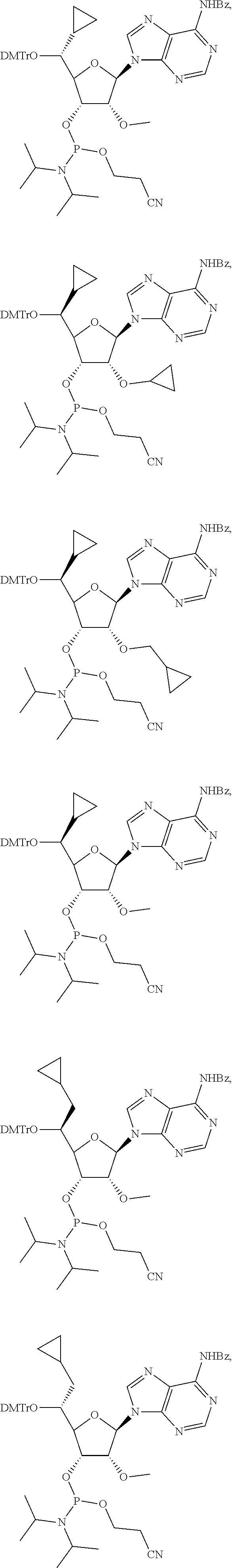

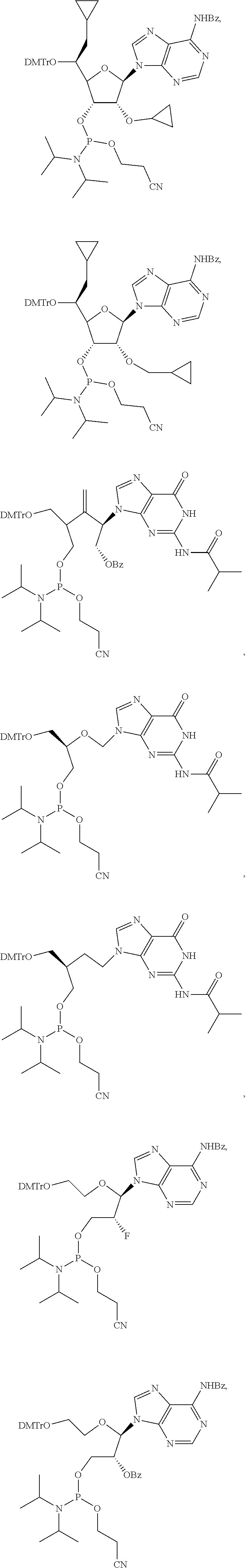

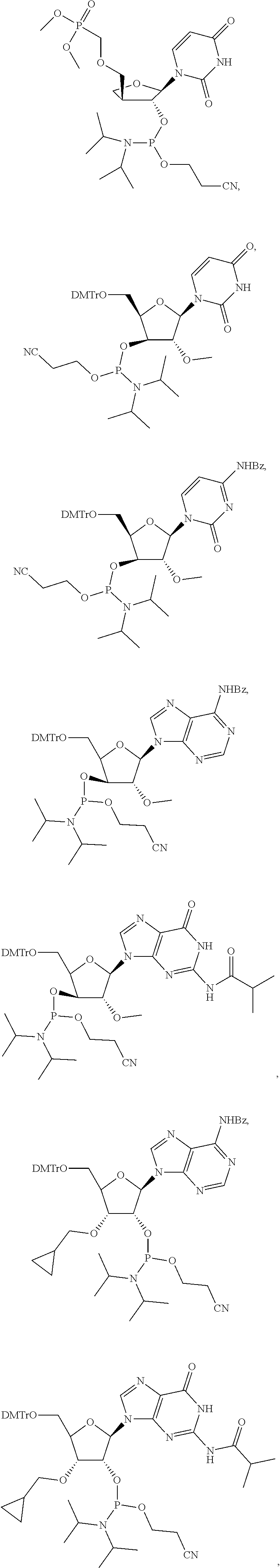

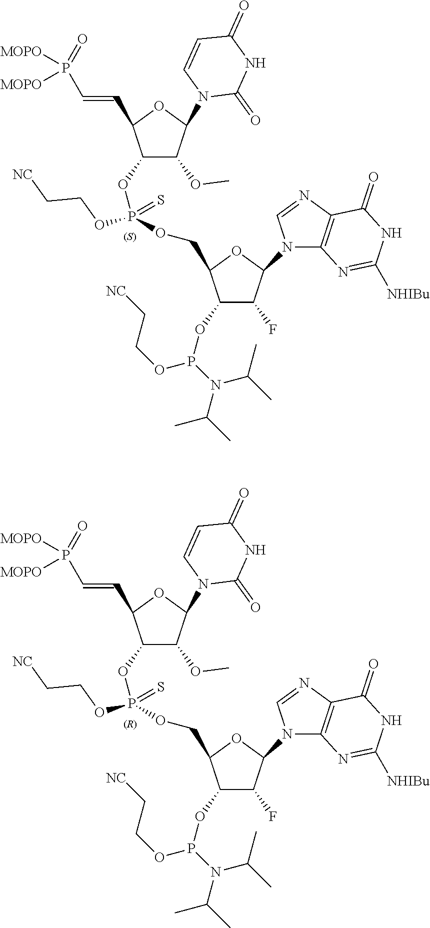

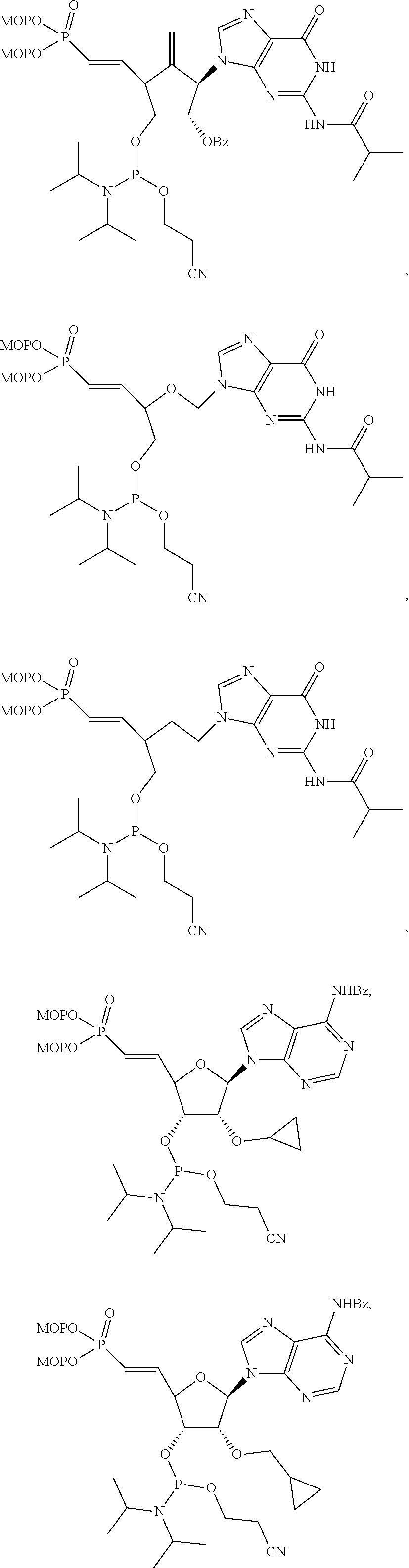

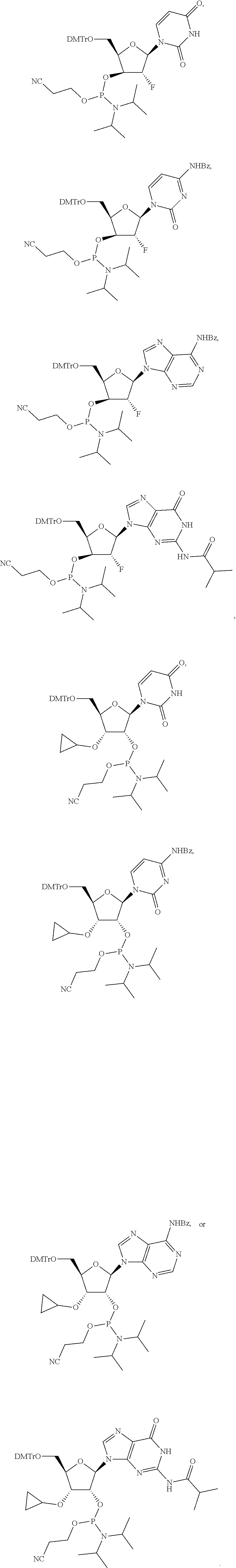

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an exemplary siNA molecule.

FIG. 2 illustrates an exemplary siNA molecule.

FIG. 3A-3J illustrate exemplary double-stranded siNA molecules. G3 represents a GalNAc conjugated moiety.

FIG. 4A-4AB illustrate exemplary double-stranded siNA molecules. G3 represents a GalNAc conjugated moiety.

FIG. 5A-SF illustrate exemplary double-stranded siNA molecules. G3 represents a GalNAc conjugated moiety.

FIG. 6A-6K illustrate exemplary double-stranded siNA molecules. G3 represents a GalNAc conjugated moiety.

FIG. 7A-7D illustrate exemplary double-stranded siNA molecules. G3 represents a GalNAc conjugated moiety.

FIG. 8A-8B illustrate exemplary double-stranded siNA molecules. G3 represents a GalNAc conjugated moiety.

FIG. 9 illustrates the in vivo activity of ds-siNAs comprising a 5′vinyl phosphonate moiety and modified unlocked nucleotides on the antisense strand. Activity was determined by measuring serum HBsAg levels assayed through ELISA. Error bars represent standard error of the mean

FIG. 10 illustrates the effects of 5′-cyclopropyl nucleotides on the stability of siNAs in mouse liver homogenate.

FIG. 11 illustrates the in vivo activity of ds-siNAs comprising 5′-cyclopropyl nucleotides on the antisense strand. Activity was determined by measuring serum HBsAg levels assayed through ELISA. Error bars represent standard error of the mean.

FIG. 12 illustrates the in vivo activity of ds-siNAs comprising 3OH and unlocked modified nucleotides on the antisense strand. Activity was determined by measuring serum HBsAg levels assayed through ELISA. Error bars represent standard error of the mean.

FIG. 13 illustrates in vivo activity of ds-siNAs comprising a 5′-end cap on the antisense strand. Activity was determined by measuring serum HBsAg levels assayed through ELISA. Error bars represent standard error of the mean.

FIG. 14A-14B illustrates (FIG. 14A) the in vitro stability measured in mouse liver homogenate, and (FIG. 14B) in vivo activity of ds-siNA analogues determined by measuring serum HBsAg levels assayed through ELISA. Error bars represent standard error of the mean.

FIG. 15A-15B illustrates (FIG. 15A) the effect of xylo modifications on the stability of siNAs in mouse liver homogenate, and (FIG. 15B) in vivo activity of ds-siNA comprising a xylo modification determined by measuring serum HBsAg levels assayed through ELISA. Error bars represent standard error of the mean.

FIG. 16A-16B illustrates (FIG. 16A) in vivo activity of ds-siNA comprising 2′F modifications along the antisense strand. Activity was determined by measuring serum HBsAg levels assayed through ELISA. Error bars represent standard error of the mean. (FIG. 16B) the effects of 2′F modifications on the stability of siNAs in mouse liver homogenate.

FIG. 17A-17B illustrates comparison in vivo activity of ds-siNA comprising 2′F modifications along the antisense strand and HBV treatment Vir-2218. Activity was determined by measuring (FIG. 17A) serum HBsAg, (FIG. 17B) alanine amino transferase (ALT) levels assayed through ELISA. Error bars represent standard error of the mean.

FIG. 18A-18C illustrates comparison in vivo activity of ds-siNA analogues and HBV treatment Vir-2218. Activity was determined by measuring (FIG. 18A) serum HBsAg, (FIG. 18B) HBeAg, and (FIG. 18C) alanine amino transferase (ALT) levels assayed through ELISA. Error bars represent standard error of the mean.

FIG. 19 illustrates in vivo activity of ds-siNA and Roch/Discerna administered at different concentrations to non-infected mice. Activity was determined by measuring serum ALT levels assayed through ELISA. Error bars represent standard error of the mean.

FIG. 20A-20B illustrates in vivo activity of ganciclovir, denvir, and 30 cp modified ds-siNA. Activity was determined by measuring (FIG. 20A) serum ALT and (FIG. 20B) serum HBsAg levels assayed through ELISA. Error bars represent standard error of the mean.

FIG. 21A-21B illustrate the effects of xylo modifications on the stability of siNAs in mouse liver homogenate.

FIG. 22A-22B illustrates in vivo activity of xylo modified ds-siNA. Activity was determined by measuring (FIG. 22A) serum HBsAg and (FIG. 22B) serum ALT levels assayed through ELISA. Error bars represent standard error of the mean.

FIG. 23A-23B illustrate the effects of stereodefined PS linkages on the stability of siNAs in mouse liver homogenate.

FIG. 24 illustrates in vivo activity of ds-siNA comprising stereodefined PS linkages. Activity was determined by measuring serum HBsAg levels assayed through ELISA. Error bars represent standard error of the mean.

FIG. 25 illustrates in vivo activity of ds-siNA comprising denavir (S) modified nucleotides on the antisense strand. Activity was determined by measuring serum HBsAg levels assayed through ELISA. Error bars represent standard error of the mean.

### DETAILED DESCRIPTION

Disclosed herein are oligonucleotide molecules (including short interfering nucleic acids or “siNAs”) comprising novel, modified nucleotide monomers and dimers that comprise a unique chemical moiety and/or other modifications. Also disclosed herein are methods of using the disclosed oligonucleotides and siNA molecules for treating various diseases and conditions.

In general, the siNA molecules described herein may be double-stranded siNA (ds-siNA) molecules. The siNA molecules described herein may comprise modified nucleotides selected from 2′-O-methyl nucleotides and 2′ fluoro nucleotides. The siNA molecules described herein may comprise 1, 2, 3, 4, 5, 6, 7, 8, 9, or 10 or more phosphorothiate internucleoside linkages. The siNA molecules described herein may comprise 1, 2, 3, 4, 5, 6, 7, 8, 9, or 10 or more mesyl phosphoramidate internucleoside linkages. The siNA molecules d scribed herein may comprise at least one phosphorylation blocker. The siNA molecules described herein may comprise a S′-stabilized end cap. The siNA molecules described herein may comprise a galactosamine. The siNA molecules described herein may comprise one or more blunt ends. The siNA molecules described herein may comprise one or more overhangs.

For instance, the present disclosure provides modified nucleotides comprising a structure of:

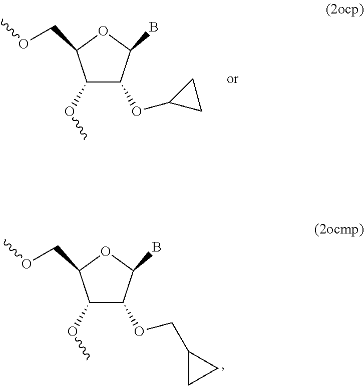

wherein B is a nucleobase, aryl, heteroaryl, or H, and wherein [symbol] represents a phosphodiester linkage, a phosphorothiate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

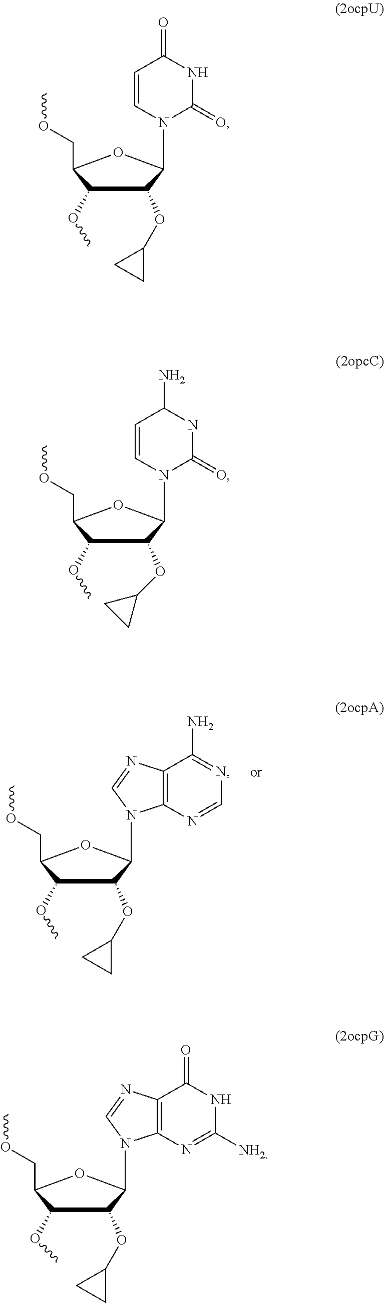

In some embodiments, the the modified nucleotides may comprise a structure of:

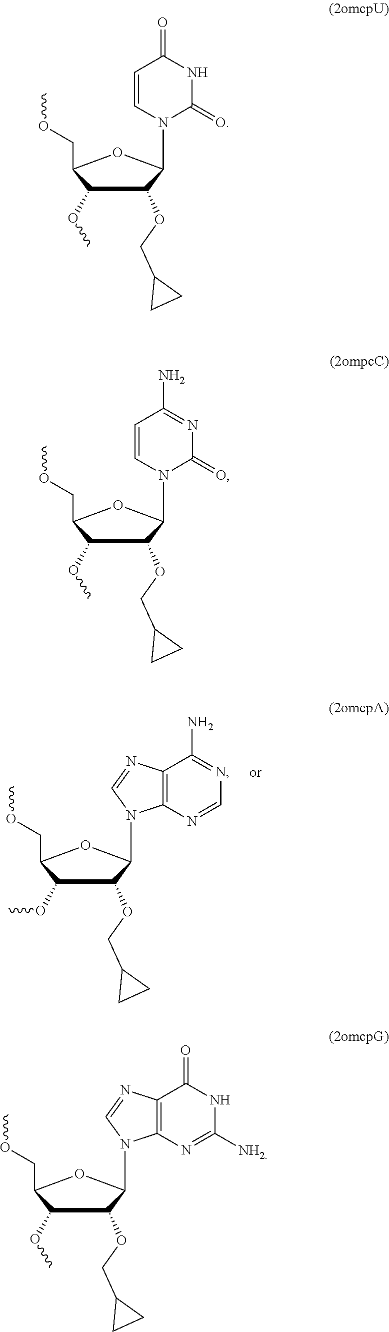

The present disclosure also provides modified nucleotides comprising a structure of:

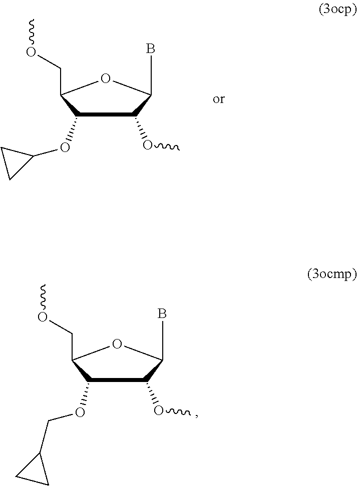

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

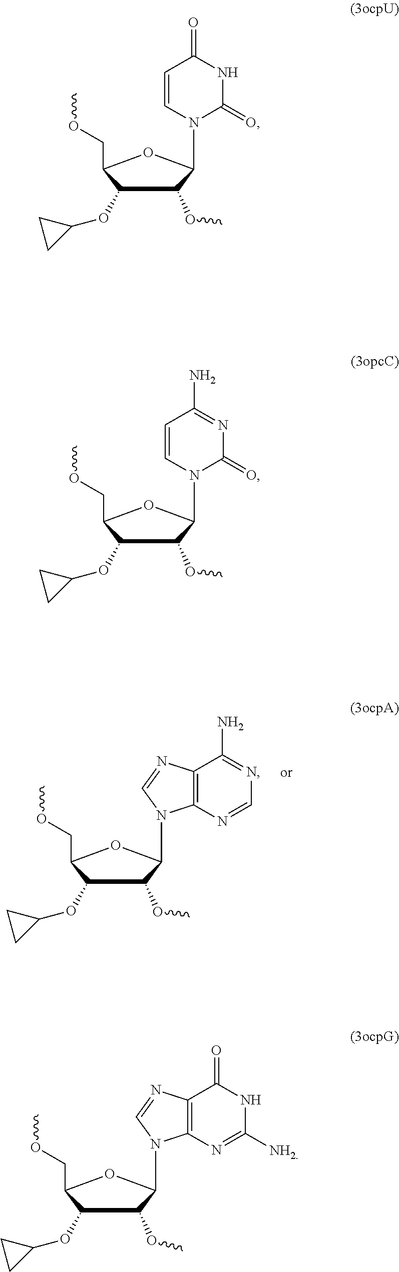

In some embodiments, the the modified nucleotides may comprise a structure of:

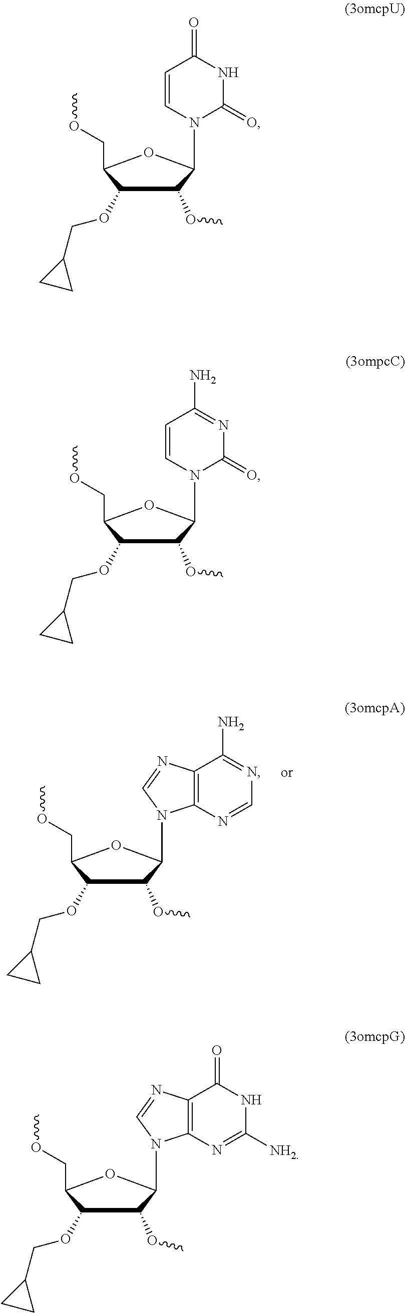

The present disclosure also provides modified nucleotides comprising a structure of:

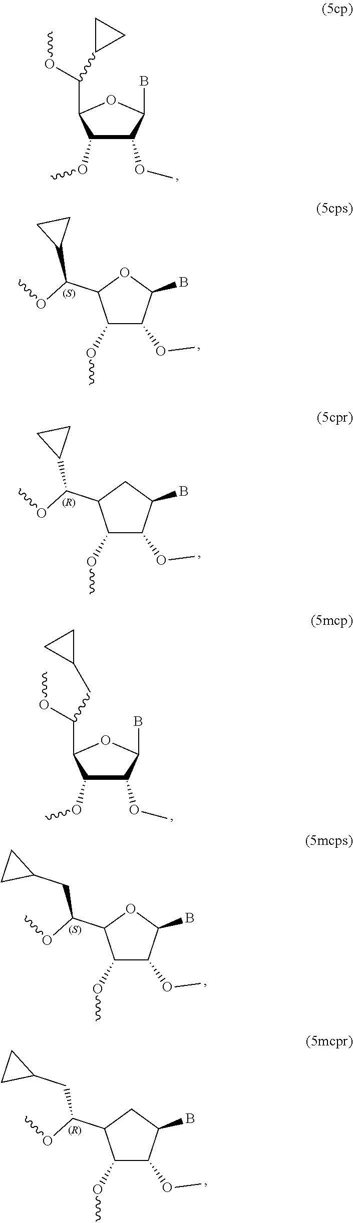

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

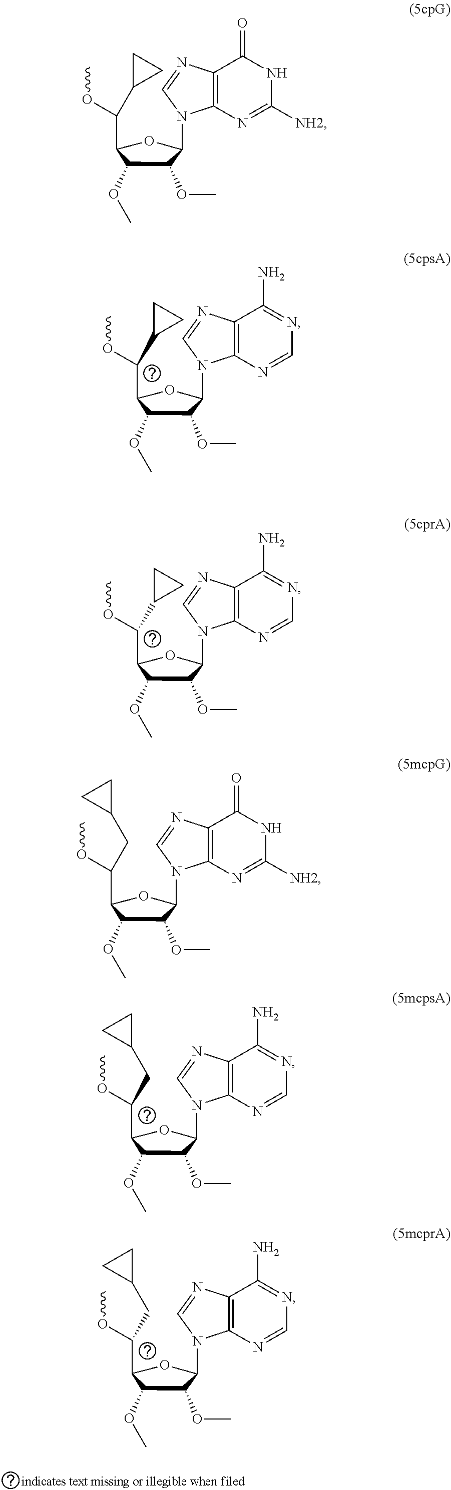

wherein A is adenine and G is guanine.

The present disclosure also provides modified nucleotides comprising a structure of:

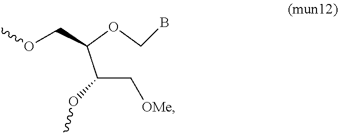

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

The present disclosure also provides modified nucleotides comprising a structure of:

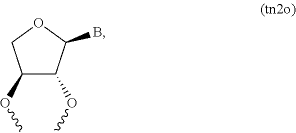

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

The present disclosure also provides modified nucleotides comprising a structure of:

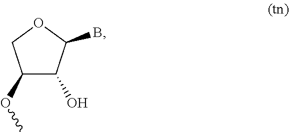

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

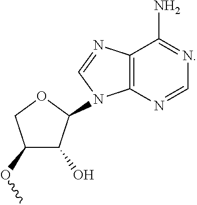

The present disclosure also provides modified nucleotides comprising a structure of:

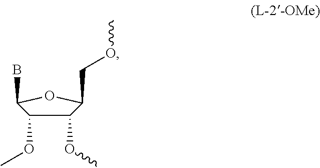

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

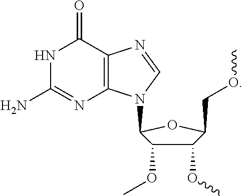

The present disclosure also provides modified nucleotides comprising a structure of:

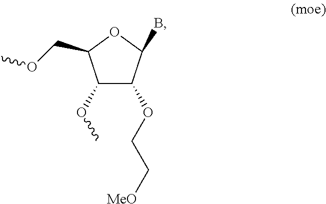

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of.

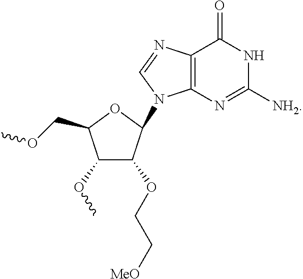

The present disclosure also provides modified nucleotides comprising a structure of:

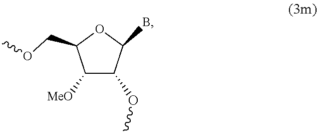

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

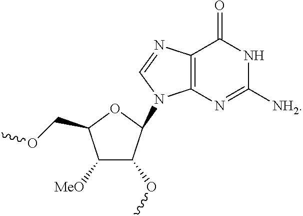

The present disclosure also provides modified nucleotides comprising a structure of:

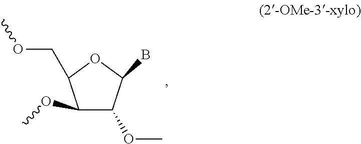

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

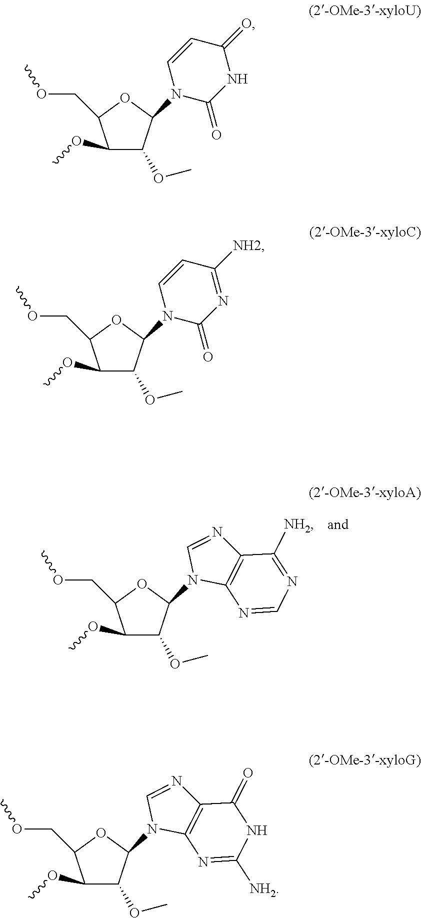

The present disclosure also provides modified nucleotides comprising a structure of:

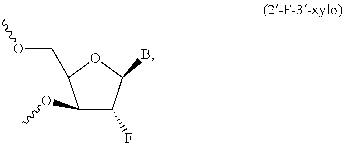

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the modified nucleotides may comprise a structure of:

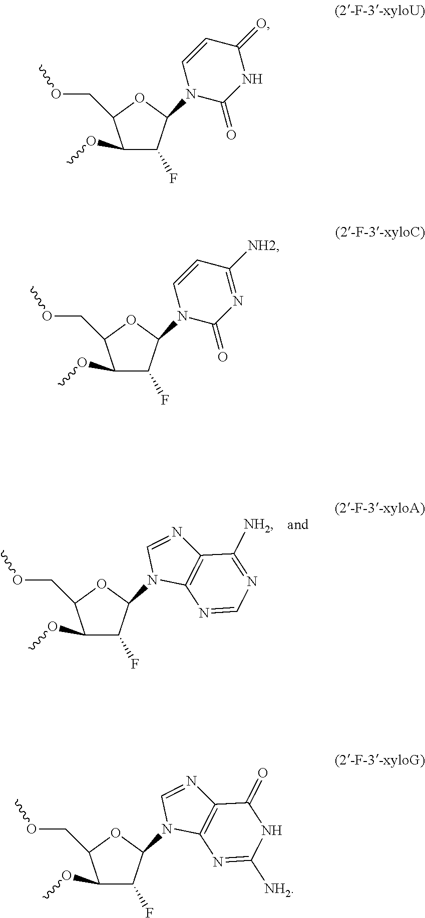

The present disclosure also provides nucleotide analogs comprising a structure of:

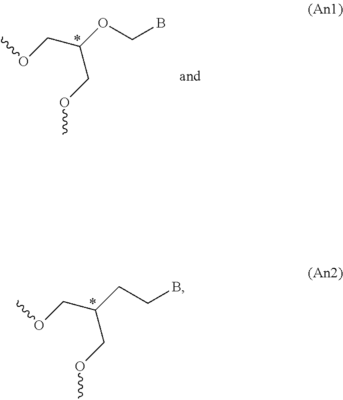

wherein B is a nucleobase, an aryl, heteroaryl, or H; wherein [symbol] represents a phosphodiester linkage, a phosphorothioate linkage, or H; and wherein * represent chiral center (e.g., R or S isomer). In some embodiments, the nucleobase is selected from thymine, cytosine, guanine, adenine, uracil, and an analog or derivative thereof.

The present disclosure further provides modified nucleotides comprising a structure of:

In a third aspect, the present disclosure provides an oligonucleotide comprising a structure selected from:

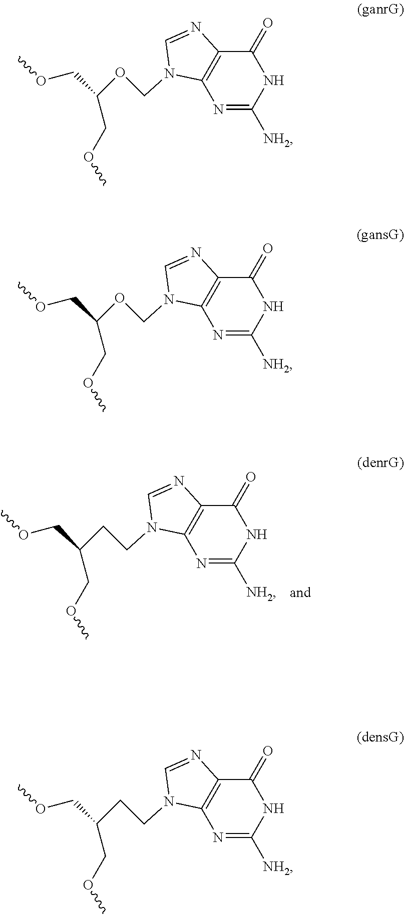

wherein B is a nucleobase, aryl, heteroaryl, or H; wherein [symbol] represents a phosphodiester linkage, a phosphorothioate linkage, or a mesyl phosphoroamidate linkage.

The present disclosure further provides modified nucleotides comprising a structure of:

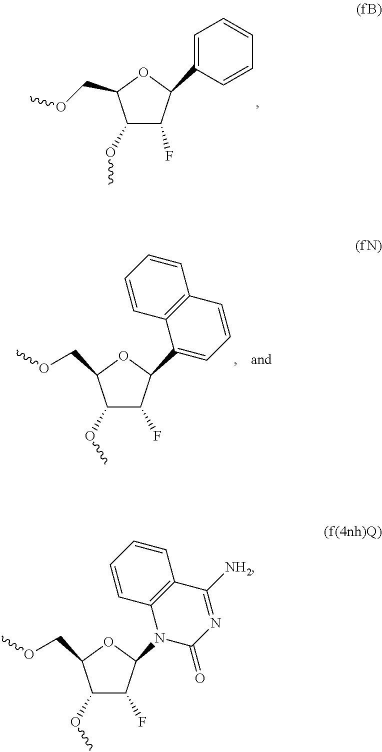

as well as modified nucleotides comprising a structure of:

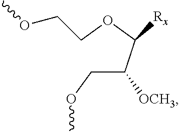

wherein Rₓ is a nucleobase, aryl, heteroaryl, or H. In some embodiments, the modified nucleotides may comprise a structure of:

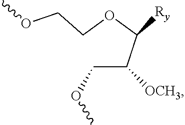

wherein R_{y} is a nucleobase. In some embodiments, the nucleobase is selected from thymine, cytosine, guanine, adenine, uracil, and an analog or derivative thereof.

The present disclosure also provides oligonucleotides comprising a structure that can serve as a stabilized end cap at the 5′ end of the antisense strand of any of the disclosed siNA. The disclosed 5′-stabilized end cap may include, but is not limited to, the structure:

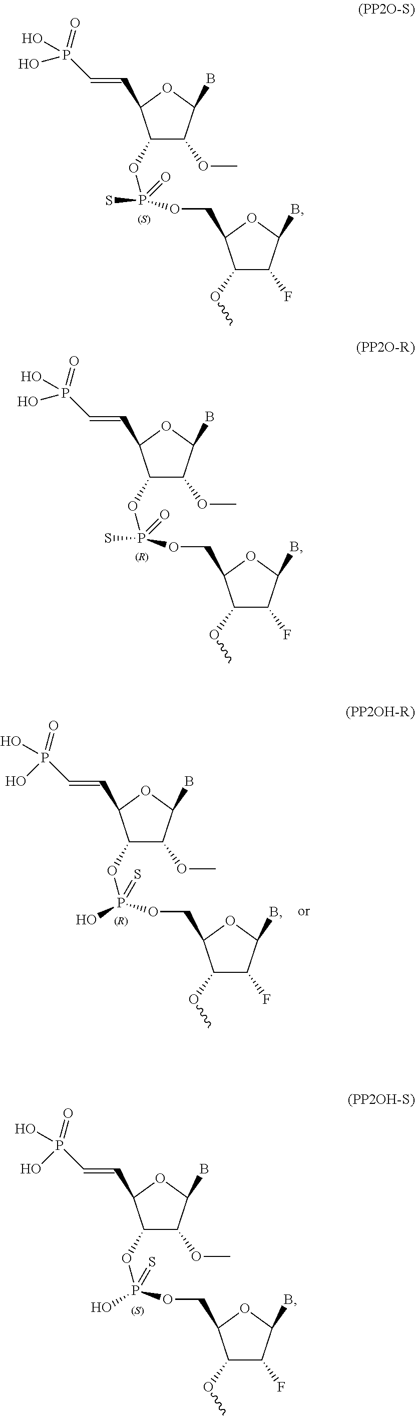

wherein each B is independently selected from a nucleobase, aryl, heteroaryl, and H; wherein β represents a phosphodiester linkage, a phosphorothioate linkage, or a mesyl phosphoroamidate linkage. In some embodiments, the nucleobase is selected from thymine, cytosine, guanine, adenine, uracil, and an analog or derivative thereof.

The present disclosure also provides structures that can serve as a stabilized end cap at the 5′ end of the antisense strand of any of the disclosed siNA. The disclosed 5′-stabilized end cap may include, but is not limited to, the structures:

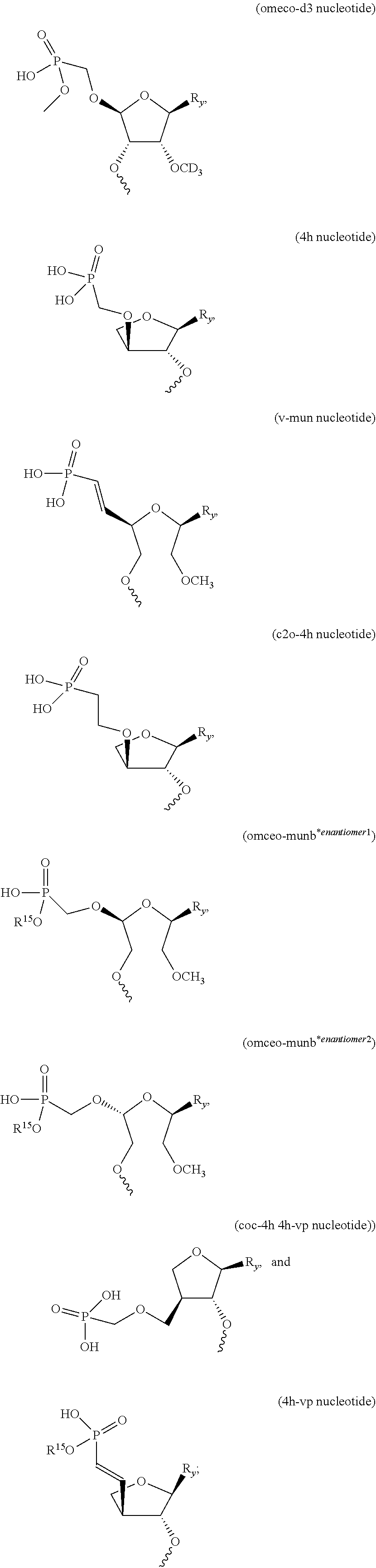

wherein R_{y} is a nucleobase and R¹⁵ is H or CH₃, and wherein [symbol] represents a phosphodiester linkage, a phosphorothioate linkage, or a mesyl phosphoroamidate linkage. In some embodiments, the nucleobase is selected from thymine, cytosine, guanine, adenine, uracil, and an analog or derivative thereof. In some embodiments, 5′-stabilized end cap may be selected from, but is not limited to, the structures:

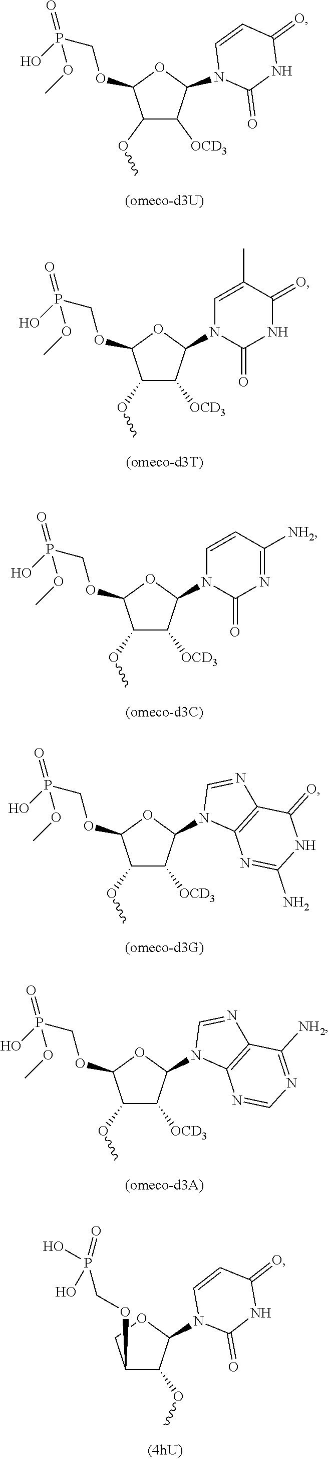

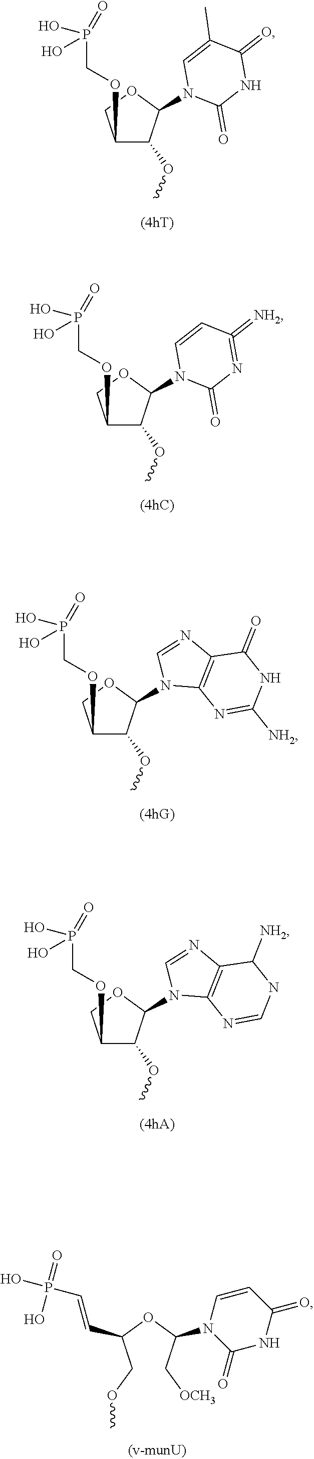

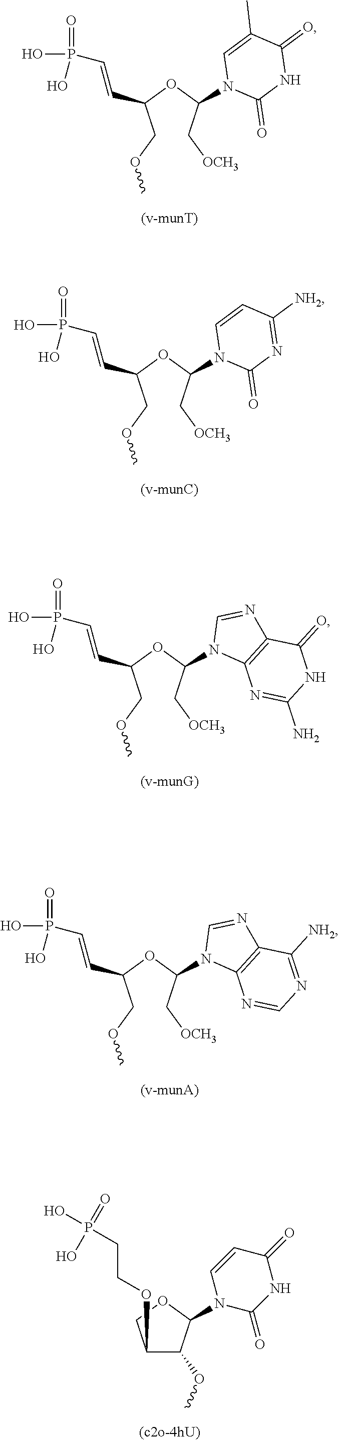

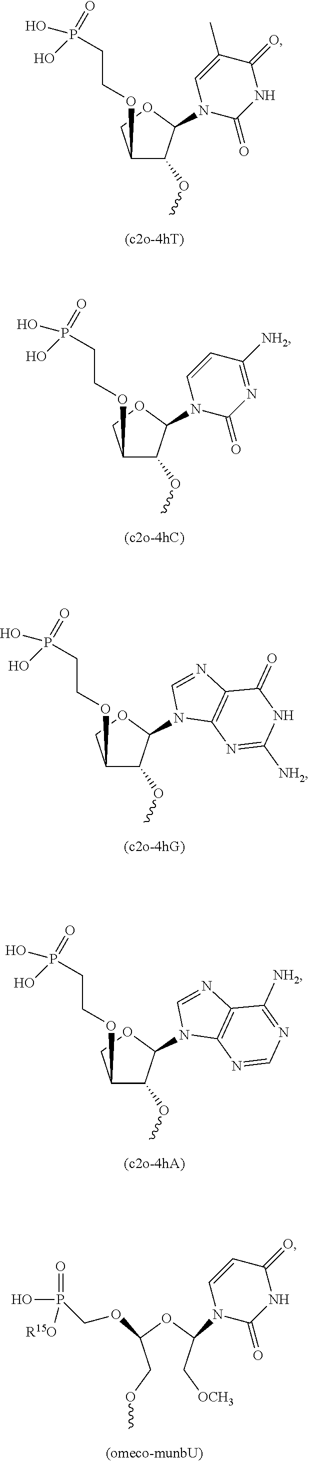

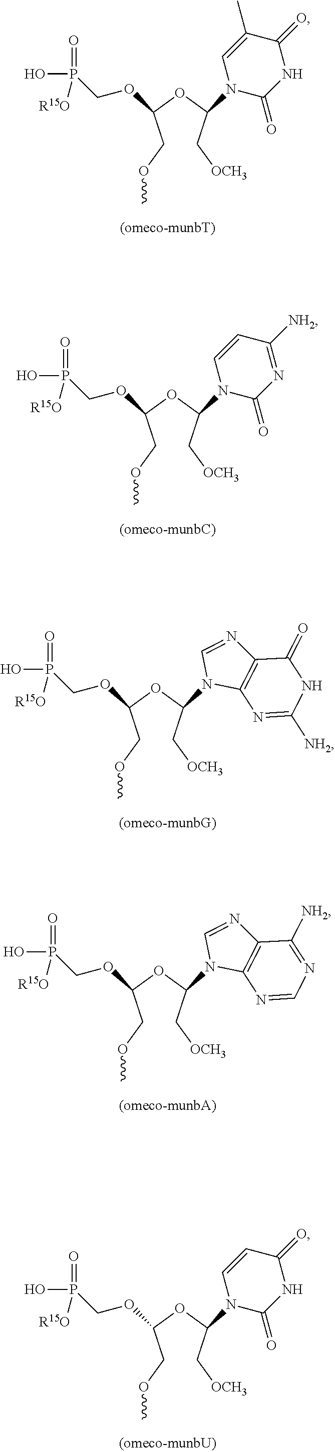

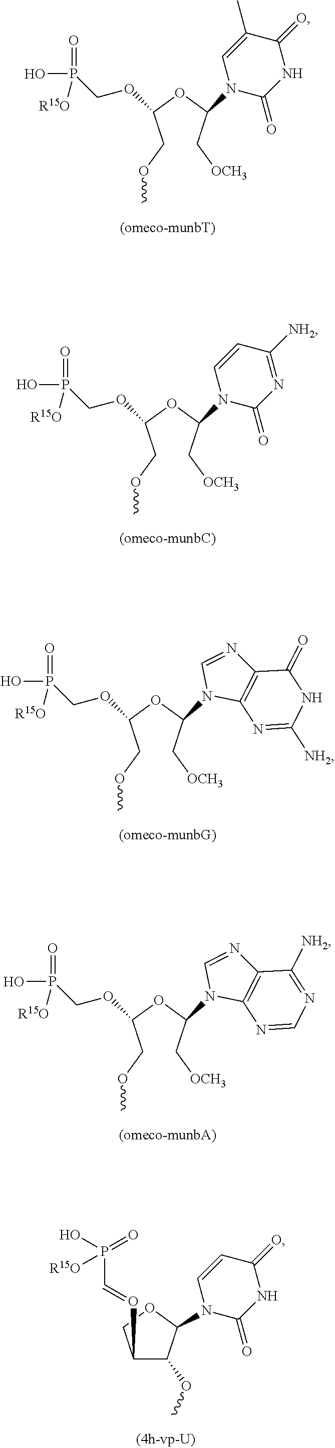

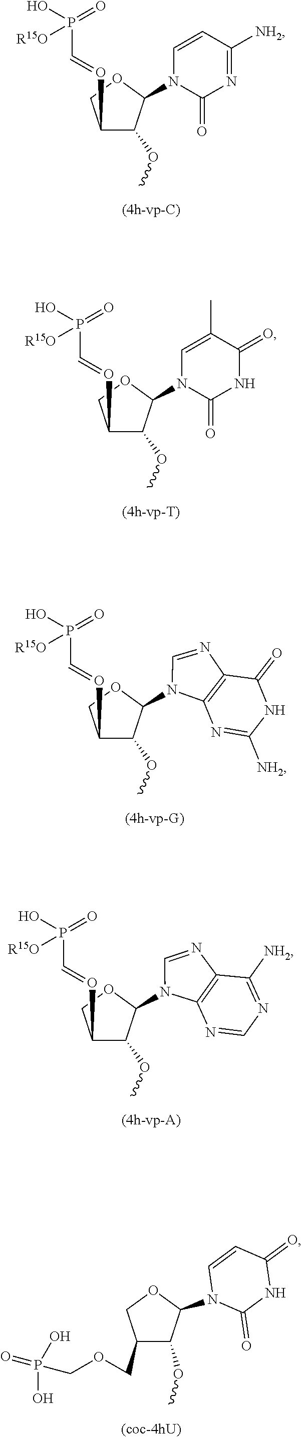

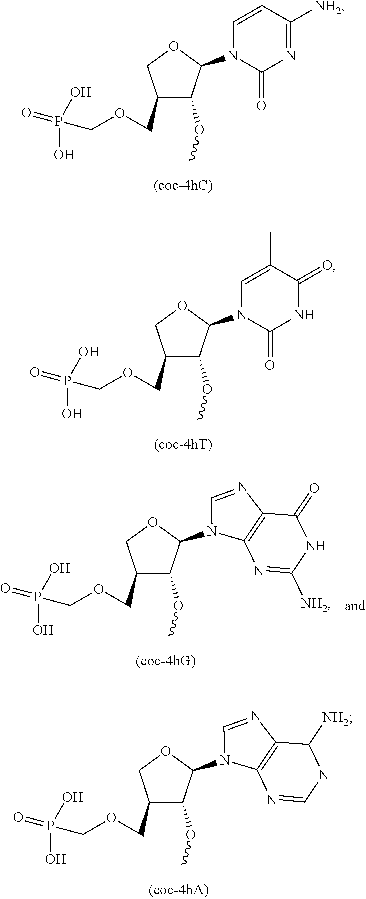

wherein R¹⁵ is H or CH₃, and wherein [symbol] represents a phosphodiester linkage, a phosphorothioate linkage, or a mesyl phosphoroamidate linkage.

The disclosed short interfering nucleic acid (siNA) molecules may comprise at least one, at least two, at least 3, at least 4, or at least 5 of the foregoing modified nucleotides and/or one of the foregoing 5′-stabilized end caps at the 5′ end of the antisense strand. Indeed, a short interfering nucleic acid (siNA) molecule of the present disclosure may comprise:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
   an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
   an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides and/or nucleotide analogs independently selected from a 2′-O-methyl nucleotide and a 2′-fluoro nucleotide, wherein at least one modified nucleotide is a 2′-O-methyl nucleotide and the nucleotide at position 2, 5, 6, 7, 8, 10, 14, 16, 17, and/or 18 from the 5′ end of the second nucleotide sequence is a 2′-fluoro nucleotide; or
(c) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (v) is 15 to 30 nucleotides in length; and
   (vi) comprises 15 or more modified nucleotides and/or nucleotide analogs independently selected from a 2′-O-methyl nucleotide and a 2′-fluoro nucleotide, wherein at least one modified nucleotide and/or nucleotide analog is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (vii) is 15 to 30 nucleotides in length; and
   (viii) comprises 15 or more modified nucleotides and/or nucleotide analog independently selected from a 2′-O-methyl nucleotide and a 2′-fluoro nucleotide, wherein at least one modified nucleotide is a 2′-O-methyl nucleotide and the nucleotide at position 2, 5, 6, 7, 8, 10, 14, 16, 17, and/or 18 from the 5′ end of the second nucleotide sequence is a 2′-fluoro nucleotide;
so long as the sense strand and/or the antisense strand comprise at least one, at least two, at least 3, at least 4, or at least 5 of the modified nucleotide(s) and/or nucleotide analog(s) selected from:

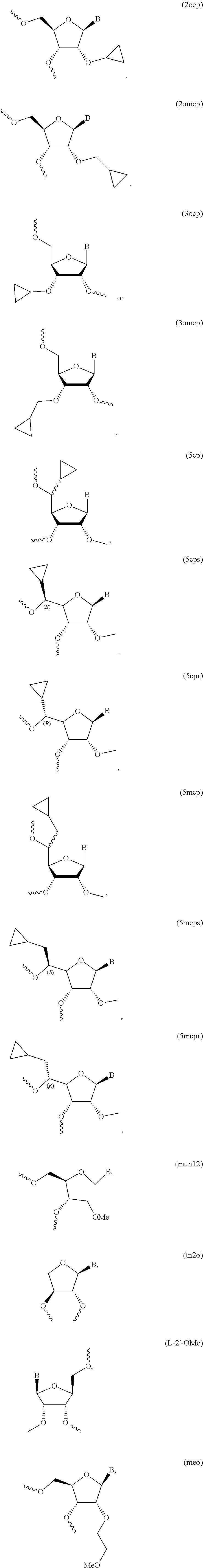

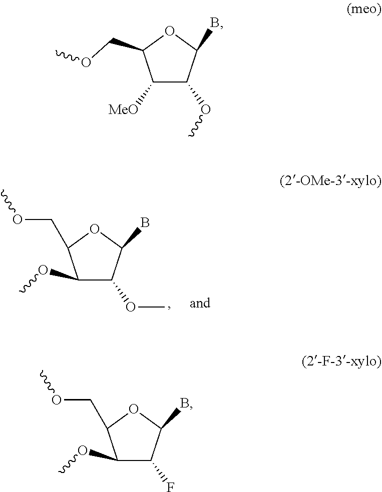

or at least 1, at least 2, at least 3, at least 4, or at least 5 nucleotide analog(s) independently selected from:

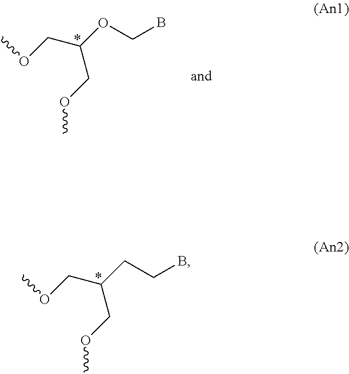

wherein B is a nucleobase, an aryl, heteroaryl, or H, wherein [symbol] represents a phosphodiester linkage, a phosphorothioate linkage, or H; and wherein * represent chiral center (e.g., R or S isomer).

Further, the siNA of the present disclosure may comprise a sense strand and/or an antisense strand that each independently comprise 1 or more phosphorothioate internucleoside linkages, 1 or more mesyl phosphoramidate internucleoside linkages, or a combination thereof. The siNA may comprise a phosphorylation blocker, a galactosamine, and/or a 5′-stabilized end cap (other than those noted above). The siNA may be conjugated to a targeting moiety, such as a galactosamine.

The present disclosure also provides siNA comprising a sense strand and an antisense strand, wherein the antisense strand comprises a 3′ overhang comprising at least one modified nucleotide selected from:

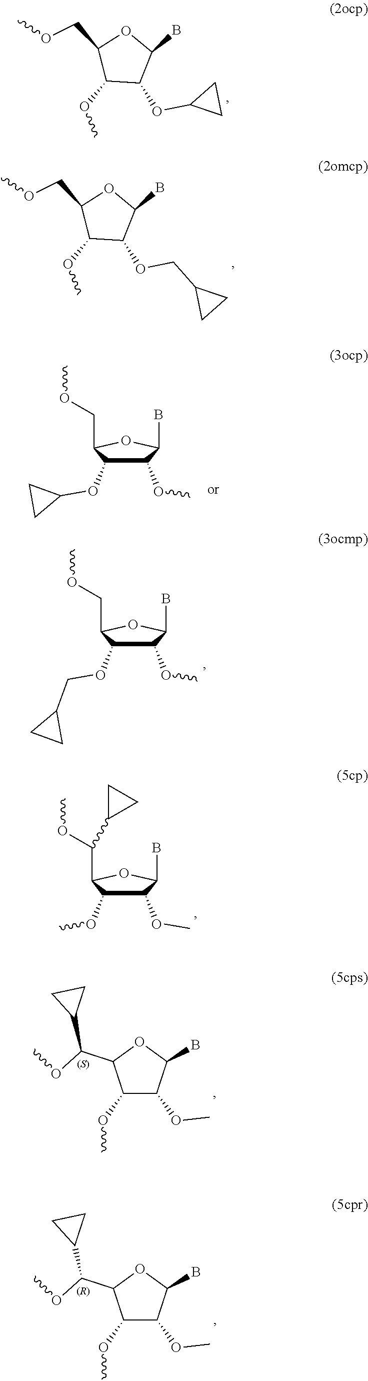

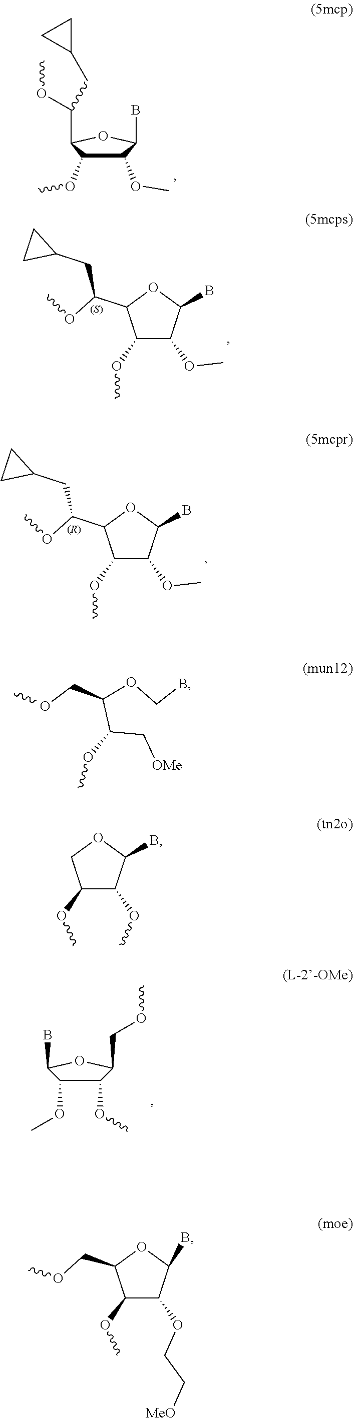

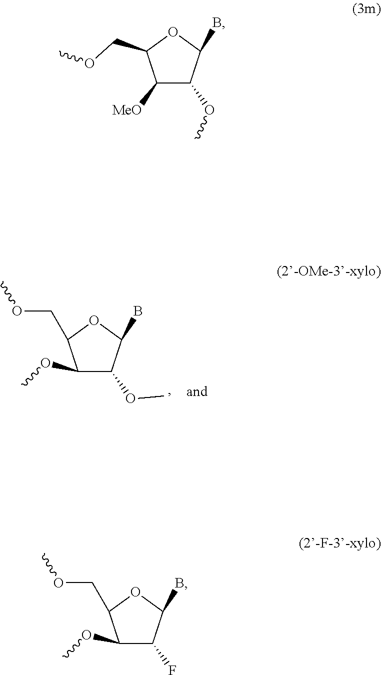

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof. In some embodiments, the disclosed nucleotide includes, but are not limited to, the structures:

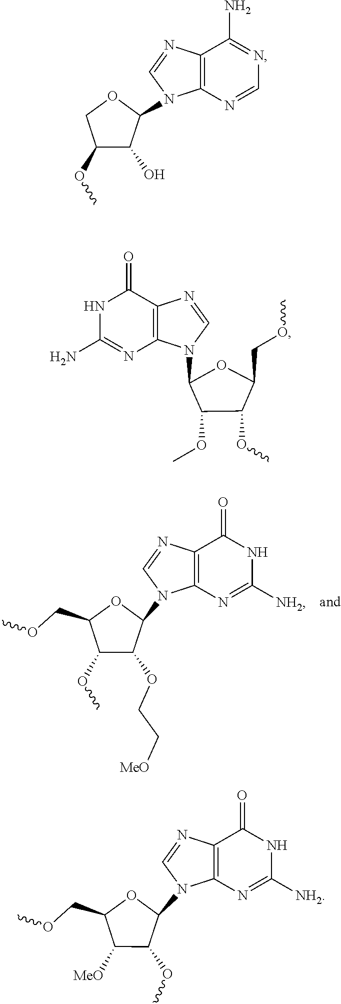

In some embodiments, the modified nucleotide is the last nucleotide or second to last nucleotide at the 3′ end of the antisense strand. In some embodiments, the siNA is resistant to nuclease activity relative to a siNA of the same sequence without the modified nucleotide in the 3′ overhang.

Further disclosed herein are compositions comprising two or more of the siNA molecules described herein.

Further disclosed herein are compositions comprising any of the siNA molecule described and a pharmaceutically acceptable carrier or diluent. Such compositions may also include an additional therapeutic agent, or may be administered in conjunction with an additional therapeutic agent (either concurrently or sequentially).

Further disclosed herein are compositions comprising two or more of the siNA molecules described herein for use as a medicament.

Further disclosed herein are compositions comprising any of the siNA molecule described and a pharmaceutically acceptable carrier or diluent for use as a medicament. Such medicaments may also include an additional therapeutic agent, or may be administered in conjunction with an additional therapeutic agent (either concurrently or sequentially).

Further disclosed herein are methods of treating a disease in a subject in need thereof, the methods comprising administering to the subject any of the siNA molecules (or a combination thereof) or compositions/medicaments described herein.

Further disclosed herein are uses of any of the siNA molecules described herein (or a combination thereof) in the manufacture of a medicament for treating a disease.

Short Interfering Nucleic Acid (siNA) Molecules

As indicated above, the present disclosure provides siNA molecules comprising modified nucleotides. Any of the siNA molecules described herein may be double-stranded siNA (ds-siNA) molecules. The terms “siNA molecules” and “ds-siNA molecules” may be used interchangeably. In some embodiments, the ds-siNA molecules comprise a sense strand and an antisense strand.

For the purposes of the present disclosure, the siNA molecules disclosed herein may generally comprise (a) at least one phosphorylation blocker, conjugated moiety, and/or 5′-stabilized end cap; and (b) a short interfering nucleic acid (siNA). In some embodiments, the phosphorylation blocker is a phosphorylation blocker disclosed herein. In some embodiments, the conjugated moiety is a galactosamine disclosed herein. In some embodiments, the 5′-stabilized end cap is a 5′-stabilized end cap disclosed herein.

The siNA may comprise any of the first nucleotide, second nucleotide, sense strand, or antisense strand sequences disclosed herein. The siNA may comprise 5 to 100, 5 to 90, 10 to 100, 10 to 90, 10 to 80, 10 to 70, 10 to 60, 10 to 50, 10 to 30, 10 to 25, 15 to 100, 15 to 90, 15 to 80, 15 to 70, 15 to 60, 15 to 50, 15 to 30, or 15 to 25 nucleotides. The siNA may comprise at least 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. The siNA may comprise less than or equal to 50, 45, 40, 39, 38, 37, 36, 35, 34, 33, 32, 31, 30, 29, 28, 27, 26, 25, 24, 23, 22, 21, 20, or 19 nucleotides. The nucleotides may be modified nucleotides. The nucleotides may be nucleotide analogs. The siNA may be single stranded (ss-siNA). The siNA may be double stranded (ds-siNA).

The ds-siNA may comprise (a) a sense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides; and (b) an antisense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides. The ds-siNA may comprise (a) a sense strand comprising about 15, 16, 17, 18, 19, 20, 21, 22, or 23 nucleotides; and (b) an antisense strand comprising about 15, 16, 17, 18, 19, 20, 21, 22, or 23 nucleotides. The ds-siNA may comprise (a) a sense strand comprising about 19 nucleotides; and (b) an antisense strand comprising about 21 nucleotides. The ds-siNA may comprise (a) a sense strand comprising about 21 nucleotides; and (b) an antisense strand comprising about 23 nucleotides.

Any of the siNA molecules disclosed herein may further comprise one or more linkers independently selected from a phosphodiester (PO) linker, phosphorothioate (PS) linker, phosphorodithioate linker, mesyl phosphoramidate (Ms), and PS-mimic linker. In some embodiments, the PS-mimic linker is a sulfur linker. In some embodiments, the linkers are internucleoside linkers. Alternatively or additionally, the linkers may connect a nucleotide of the siNA molecule to at least one phosphorylation blocker, conjugated moiety, or 5′-stabilized end cap. In some embodiments, the linkers connect a conjugated moiety to a phosphorylation blocker or 5′-stabilized end cap.

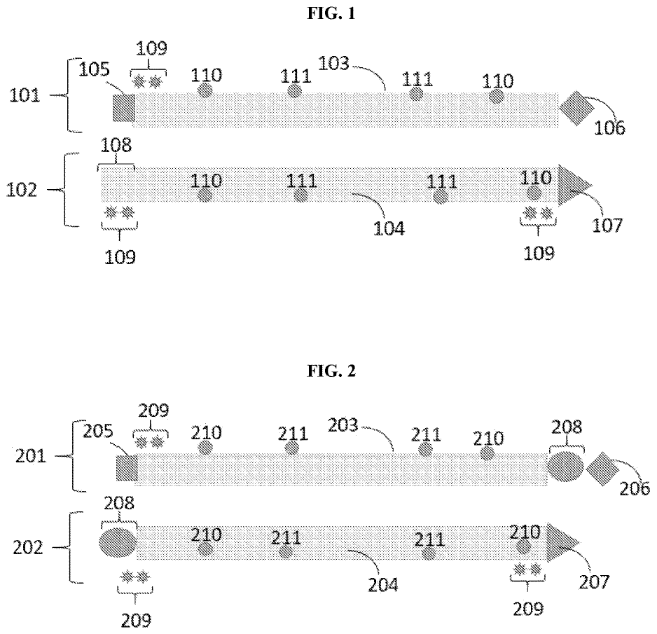

An exemplary siNA molecule of the present disclosure is shown in FIG. 1. As shown in FIG. 1, an exemplary siNA molecule comprises a sense strand (101) and an antisense strand (102). The sense strand (101) may comprise a first oligonucleotide sequence (103). The first oligonucleotide sequence (103) may comprise one or more phosphorothioate internucleoside linkages (109). The phosphorothioate internucleoside linkage (109) may be between the nucleotides at the 5′ or 3′ terminal end of the first oligonucleotide sequence (103). The phosphorothioate internucleoside linkage (109) may be between the first three nucleotides from the 5′ end of the first oligonucleotide sequence (103). The first oligonucleotide sequence (103) may comprise one or more 2′-fluoro nucleotides (110). The first oligonucleotide sequence (103) may comprise one or more 2′-O-methyl nucleotides (111). The first oligonucleotide sequence (103) may comprise 15 or more modified nucleotides independently selected from 2′-fluoro nucleotides (110) and 2′-O-methyl nucleotides (111). The sense strand (101) may further comprise a phosphorylation blocker (105). The sense strand (101) may further comprise a galactosamine (106). The antisense strand (102) may comprise a second oligonucleotide sequence (104). The second oligonucleotide sequence (104) may comprise one or more phophorothioate internucleoside linkages (109). The phosphorothioate internucleoside linkage (109) may be between the nucleotides at the 5′ or 3′ terminal end of the second oligonucleotide sequence (104). The phosphorothioate internucleoside linkage (109) may be between the first three nucleotides from the 5′ end of the second oligonucleotide sequence (104). The phosphorothioate internucleoside linkage (109) may be between the first three nucleotides from the 3′ end of the second oligonucleotide sequence (104). The second oligonucleotide sequence (104) may comprise one or more 2′-fluoro nucleotides (110). The second oligonucleotide sequence (104) may comprise one or more 2′-O-methyl nucleotides (111). The second oligonucleotide sequence (104) may comprise 15 or more modified nucleotides independently selected from 2′-fluoro nucleotides (110) and 2′-O-methyl nucleotides (111). The antisense strand (102) may further comprise a 5′-stabilized end cap (107). The siNA may further comprise one or more blunt ends. Alternatively, or additionally, one end of the siNA may comprise an overhang (108). The overhang (108) may be part of the sense strand (101). The overhang (108) may be part of the antisense strand (102). The overhang (108) may be distinct from the first nucleotide sequence (103). The overhang (108) may be distinct from the second nucleotide sequence (104). The overhang (108) may be part of the first nucleotide sequence (103). The overhang (108) may be part of the second nucleotide sequence (104). The overhang (108) may comprise 1 or more nucleotides. The overhang (108) may comprise 1 or more deoxyribonucleotides. The overhang (108) may comprise 1 or more modified nucleotides. The overhang (108) may comprise 1 or more modified ribonucleotides. The sense strand (101) may be shorter than the antisense strand (102). The sense strand (101) may be the same length as the antisense strand (102). The sense strand (101) may be longer than the antisense strand (102).

An exemplary siNA molecule of the present disclosure is shown in FIG. 2. As shown in FIG. 2, an exemplary siNA molecule comprises a sense strand (201) and an antisense strand (202). The sense strand (201) may comprise a first oligonucleotide sequence (203). The first oligonucleotide sequence (203) may comprise one or more phophorothioate internucleoside linkages (209). The phosphorothioate internucleoside linkage (209) may be between the nucleotides at the 5′ or 3′ terminal end of the first oligonucleotide sequence (203). The phosphorothioate internucleoside linkage (209) may be between the first three nucleotides from the 5′ end of the first oligonucleotide sequence (203). The first oligonucleotide sequence (203) may comprise one or more 2′-fluoro nucleotides (210). The first oligonucleotide sequence (203) may comprise one or more 2′-O-methyl nucleotides (211). The first oligonucleotide sequence (203) may comprise 15 or more modified nucleotides independently selected from 2′-fluoro nucleotides (210) and 2′-O-methyl nucleotides (211). The sense strand (201) may further comprise a phosphorylation blocker (205). The sense strand (201) may further comprise a galactosamine (206). The antisense strand (202) may comprise a second oligonucleotide sequence (204). The second oligonucleotide sequence (204) may comprise one or more phophorothioate internucleoside linkages (209). The phosphorothioate internucleoside linkage (209) may be between the nucleotides at the 5′ or 3′ terminal end of the second oligonucleotide sequence (204). The phosphorothioate internucleoside linkage (209) may be between the first three nucleotides from the 5′ end of the second oligonucleotide sequence (204). The phosphorothioate internucleoside linkage (209) may be between the first three nucleotides from the 3′ end of the second oligonucleotide sequence (204). The second oligonucleotide sequence (204) may comprise one or more 2′-fluoro nucleotides (210). The second oligonucleotide sequence (204) may comprise one or more 2′-O-methyl nucleotides (211). The second oligonucleotide sequence (204) may comprise 15 or more modified nucleotides independently selected from 2′-fluoro nucleotides (210) and 2′-O-methyl nucleotides (211). The antisense strand (202) may further comprise a 5′-stabilized end cap (207). The siNA may further comprise one or more overhangs (208). The overhang (208) may be part of the sense strand (201). The overhang (208) may be part of the antisense strand. (202). The overhang (208) may be distinct from the first nucleotide sequence (203). The overhang (208) may be distinct from the second nucleotide sequence (204). The overhang (208) may be part of the first nucleotide sequence (203). The overhang (208) may be part of the second nucleotide sequence (204). The overhang (208) may be adjacent to the 3′ end of the first nucleotide sequence (203). The overhang (208) may be adjacent to the 5′ end of the first nucleotide sequence (203). The overhang (208) may be adjacent to the 3′ end of the second nucleotide sequence (204). The overhang (208) may be adjacent to the 5′ end of the second nucleotide sequence (204). The overhang (208) may comprise 1 or more nucleotides. The overhang (208) may comprise 1 or more deoxyribonucleotides. The overhang (208) may comprise a TT sequence. The overhang (208) may comprise 1 or more modified nucleotides. The overhang (208) may comprise 1 or more modified nucleotides disclosed herein (e.g., 2-fluoro nucleotide, 2′-O-methyl nucleotide, 2′-fluoro nucleotide mimic, 2′-O-methyl nucleotide mimic, or a nucleotide comprising a modified nucleobase). The overhang (208) may comprise 1 or more modified ribonucleotides. The sense strand (201) may be shorter than the antisense strand (202). The sense strand (201) may be the same length as the antisense strand (202). The sense strand (201) may be longer than the antisense strand (202).

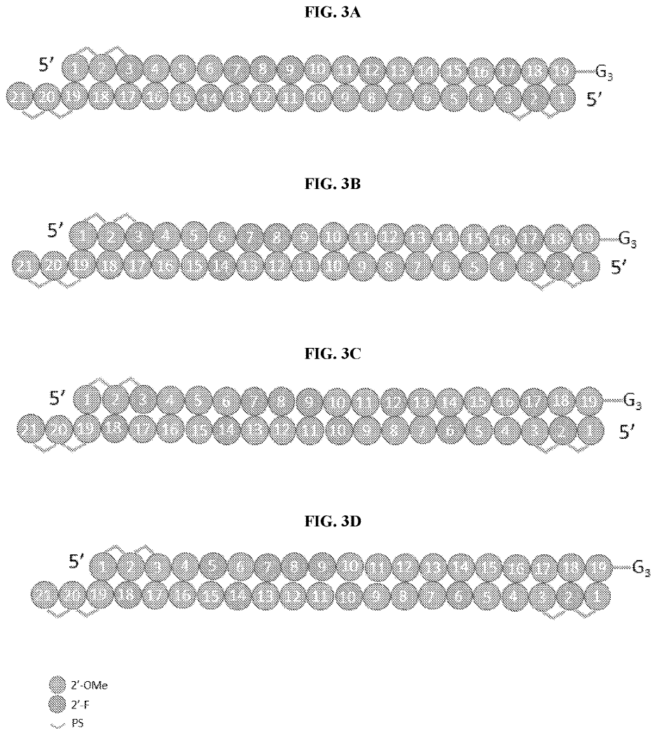

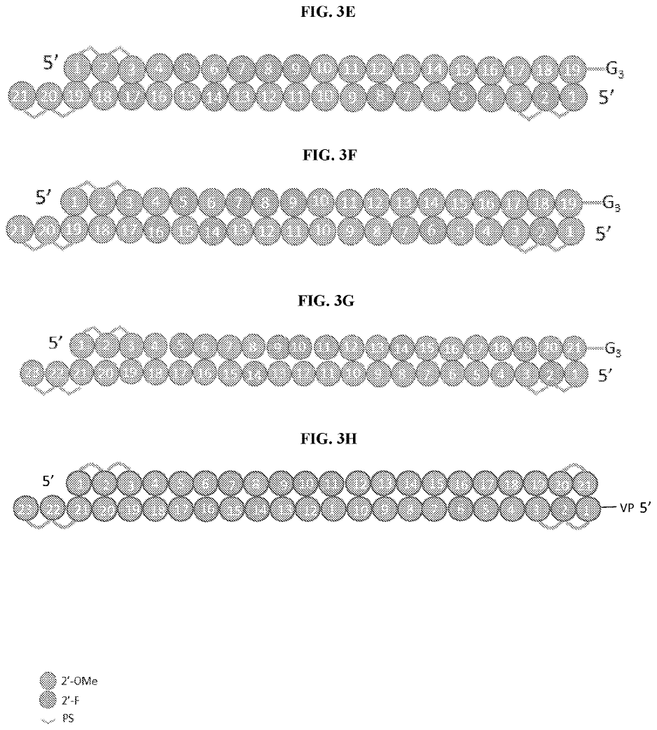

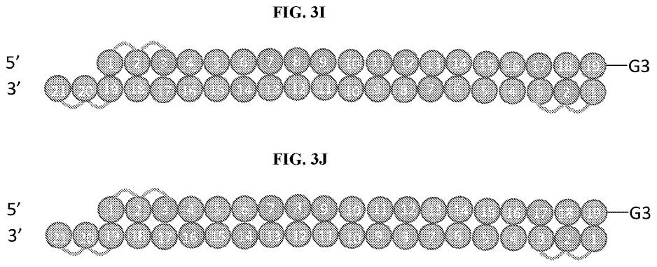

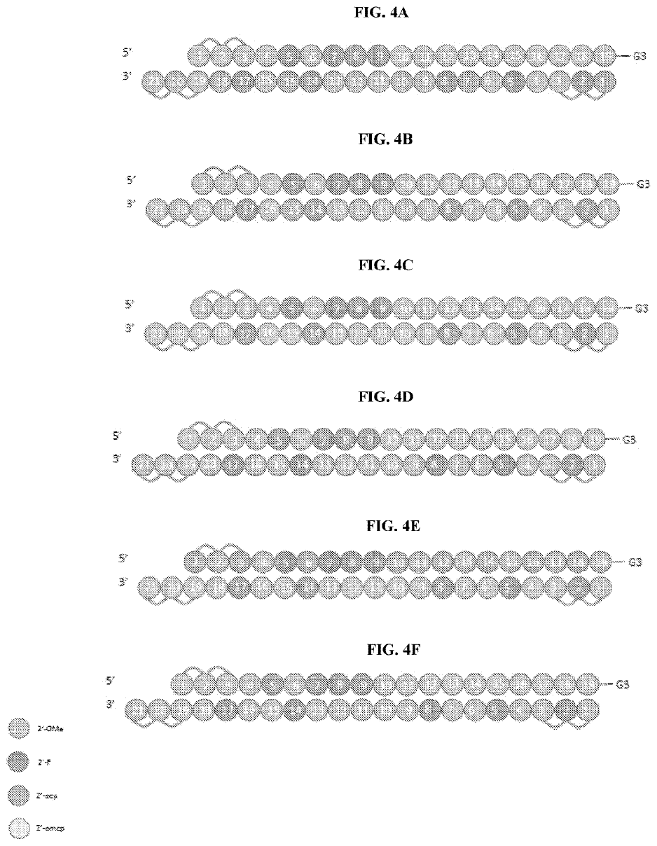

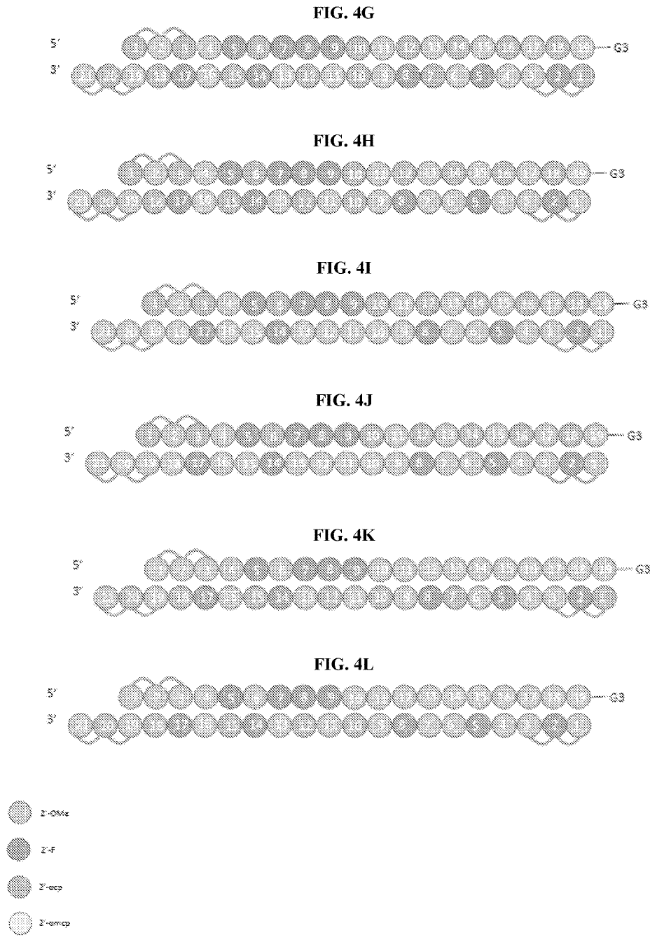

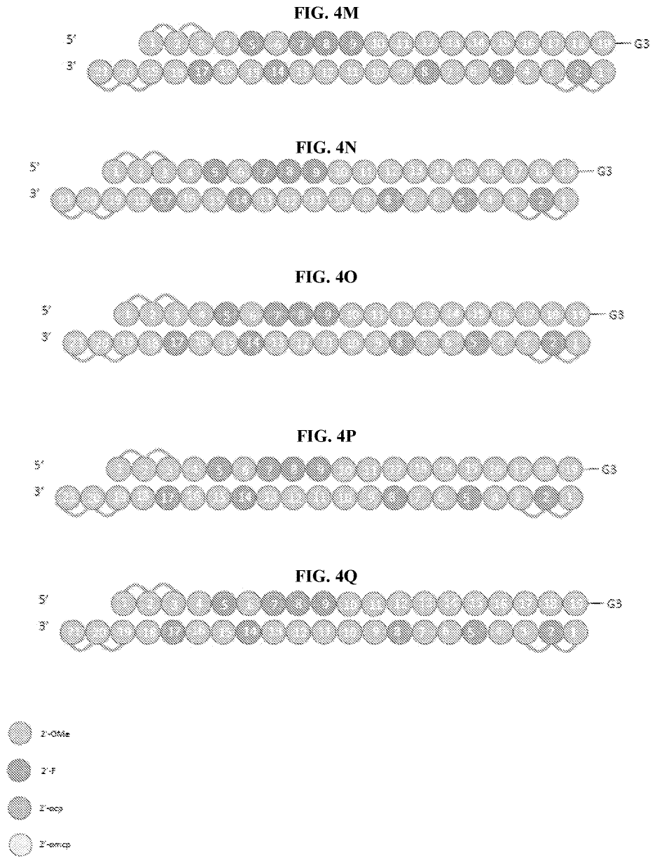

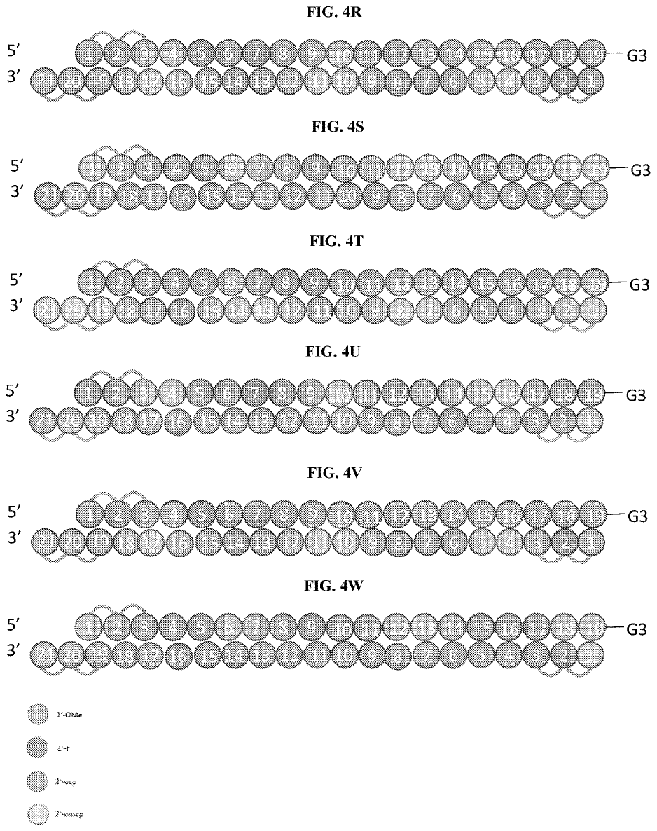

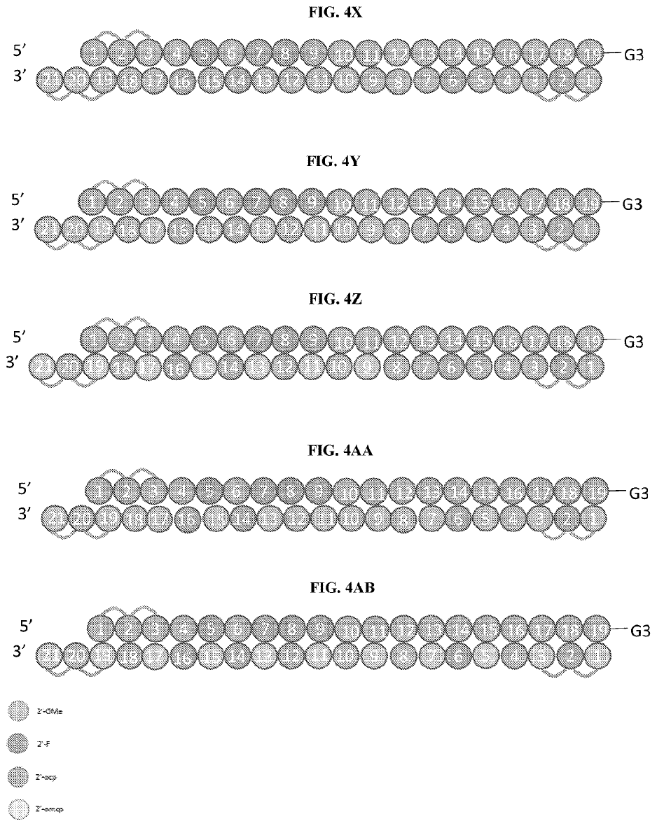

FIGS. 3A-3J, 4A-4AB, 5A-F, 6A-K, 7A-D, and 8A-B depict exemplary ds-siNA modification patterns. As shown in FIGS. 3A-3J, an exemplary ds-siNA molecule may have the following formula:

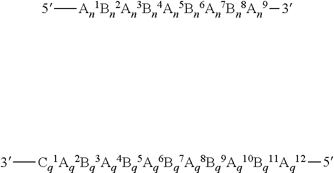

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide, a deoxy nucleotide, or uracil;
n¹=1-6 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length.

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) The ds-siNA may further comprise a 5′-stabilizing end cap. The 5′-stabilizing end cap may be a vinyl phosphonate. The 5′-stabilizing end cap may be attached to the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. An exemplary ds-siNA molecule may have the following formula:

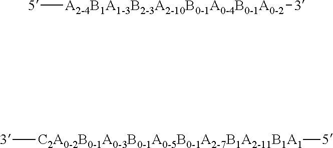

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′ stabilized end cap or phosphorylation blocker;
B is a 2′-fluoro nucleotide;
C represents overhanging nucleotides and is a 2′-O-methyl nucleotide, a deoxy nucleotide, or uracil.

The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) The ds-siNA may further comprise a 5′-stabilizing end cap. The 5′-stabilizing end cap may be a vinyl phosphonate. The vinyl phosphonate may be a deuterated vinyl phosphonate. The deuterated vinyl phosphonate may be a mono-deuterated vinyl phosphonate. The deuterated vinyl phosphonate may be a mono-di-deuterated vinyl phosphonate. The 5′-stabilizing end cap may be attached to the 5′ end of the antisense strand. The 5′-stabilizing end cap may be attached to the 3′ end of the antisense strand. The 5′-stabilizing end cap may be attached to the 5′ end of the sense strand. The 5′-stabilizing end cap may be attached to the 3′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

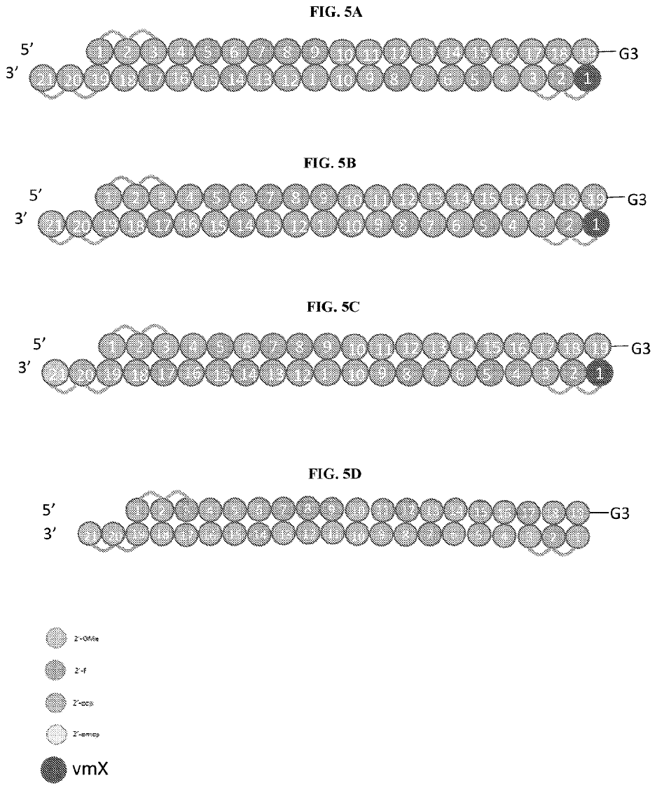

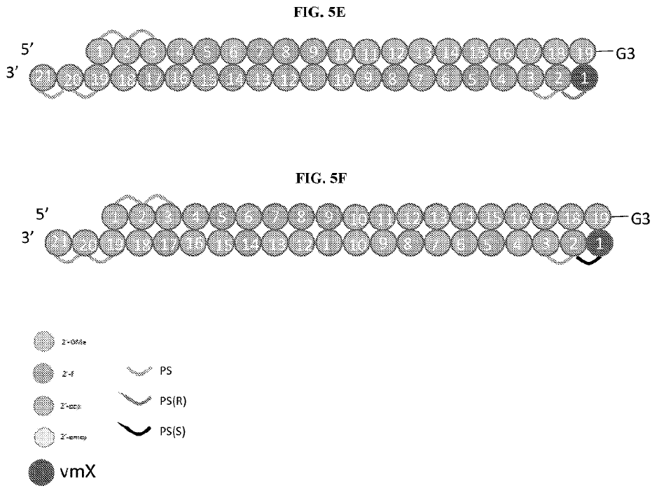

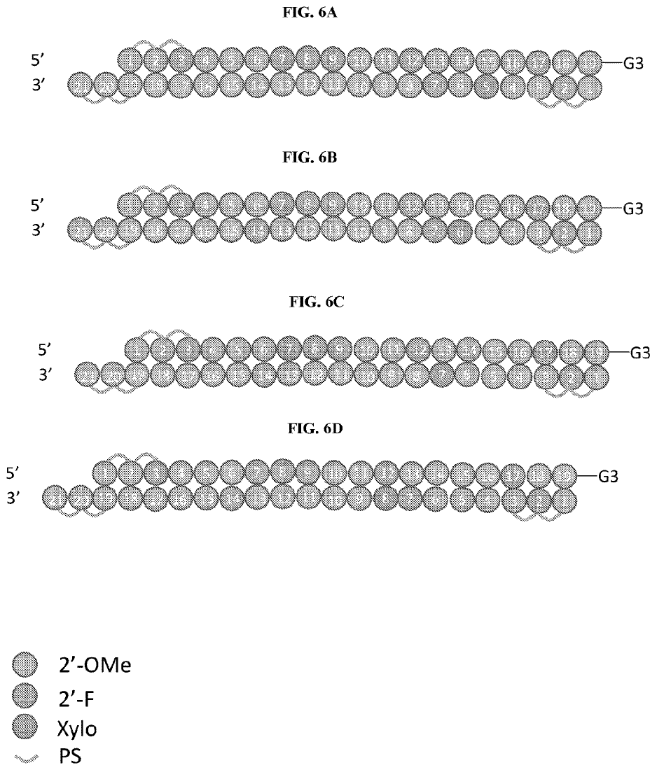

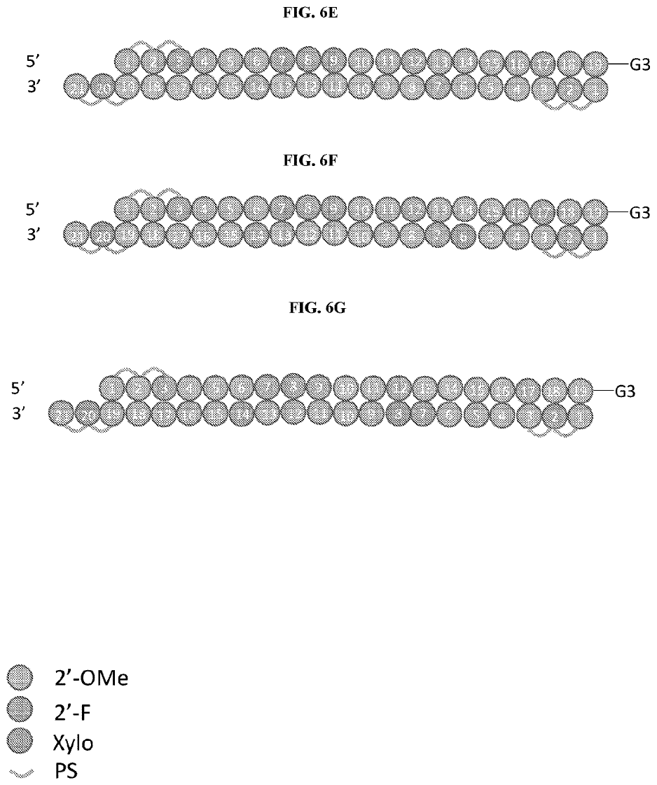

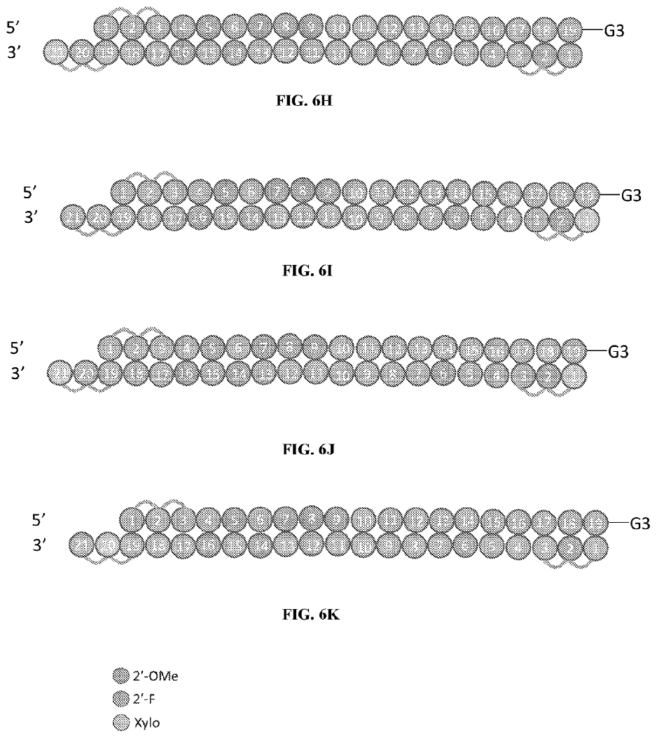

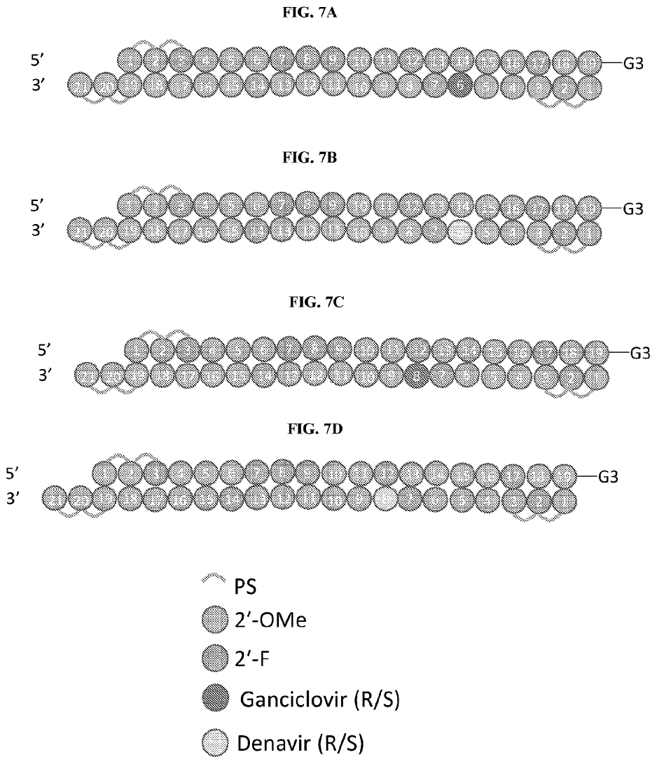

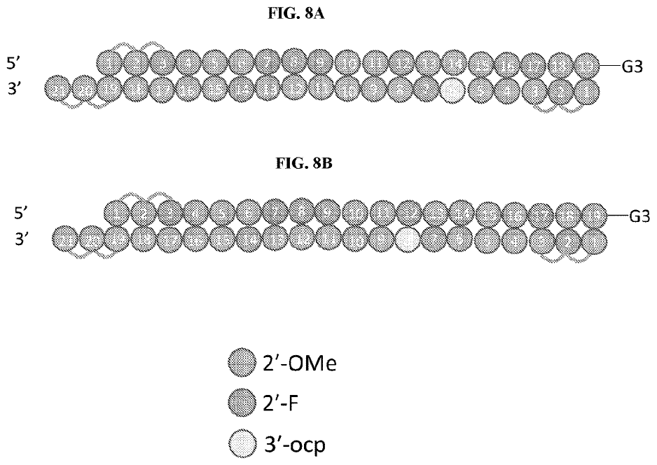

The exemplary ds-siNA shown in FIGS. 3A-3J, 4A-4AB, 5A-F, 6A-K, 7A-D, and 8A-B comprise (i) a sense strand comprising 19-21 nucleotides; and (ii) an antisense strand comprising 21-23 nucleotides. The ds-siNA may optionally further comprise (iii) a conjugated moiety, wherein the conjugated moiety (e.g., a GalNAc, noted as G3 in the FIGs.) is attached to the 3′ end or the 5′ end of the sense strand or the antisense strand. The ds-siNA may comprise a 2 nucleotide overhang consisting of nucleotides at positions 20 and 21 from the 5′ end of the antisense strand. The ds-siNA may comprise a 2 nucleotide overhang consisting of nucleotides at positions 22 and 23 from the 5′ end of the antisense strand. The ds-siNA may further comprise 1, 2, 3, 4, 5, 6 or more phosphorothioate (ps) internucleoside linkages or mesyl phosphoramidate internucleoside linkage (Ms). At least one phosphorothioate internucleoside linkage or mesyl phosphoramidate internucleoside linkage (Ms) may be between the nucleotides at positions 1 and 2 or positions 2 and 3 from the 5′ end of the sense strand. At least one phosphorothioate internucleoside linkage or mesyl phosphoramidate internucleoside linkage (Ms) may be between the nucleotides at positions 1 and 2 or positions 2 and 3 from the 5′ end of the antisense strand. At least one phosphorothioate internucleoside linkage or mesyl phosphoramidate internucleoside linkage (Ms) may be between the nucleotides at positions 19 and 20, positions 20 and 21, positions 21 and 22, or positions 22 and 23 from the 5′ end of the antisense strand. As shown in FIGS. 3A-3J, 4A-4AB, 5A-F, 6A-K, 7A-D, and &A-B, 4-6 nucleotides in the sense strand may be 2′-fluoro nucleotides. As shown in FIGS. 3A-3J, 4A-4AB, 5A-F, 6A-K, 7A-D, and 8A-B, 2-5 nucleotides in the antisense strand may be 2′-fluoro nucleotides. As shown in FIGS. 3A-3J, 4A-4D, 4P, 4R-4AB, 5A-F, 6A-K, 7A-D, and 8A-B, 13-15 nucleotides in the sense strand may be 2′-O-methyl nucleotides. As shown in FIGS. 3A-3J, 4E, 4F, 40, 4R-X, 5A-F, 6A-K, 7A-D, and 8A-B, 14-19 nucleotides in the antisense strand may be 2′-O-methyl nucleotides. As shown in FIGS. 4E and 4G-4J, up to 8 nucleotides (i.e., 1, 2, 3, 4, 5, 6, 7, 8) in the sense strand may be 2′-O-cyclopropane (2′-ocp). As shown in FIGS. 4A, 4B, 4G, 4H, 4K, 4L, 4R, 4S, 4V, 4X, 4Y, 4AA, 5B, and 5D, up to 11 nucleotides (i.e., 1, 2, 3, 4, 5, 6, 7, 8, 9, 10, 11) in the antisense strand may be 2′-ocp. As shown in FIGS. 4F, 4K-4N, and 4Q, 9-15 nucleotides in the sense strand may be 2′-OMe-cyclopropane (2′-omcp). As shown in FIGS. 4J, 4M, 4N, 4P 4Q, 4T, 4U, 4W, 4Z, 4AB, and 5C, 1-15 nucleotides in the sense strand may be 2′-omcp. As shown in FIGS. 5A-C, E, and F, position 1 from the 5′ end of the antisense strand may be vmX. As shown in FIGS. 6A-6G, one or two nucleotides in the antisense strand may be xylo nucleotides, i.e., 2′-OMe-3′-xylo or 2′-F-3′-xylo nucleotides. As shown in FIGS. 7A-D, one nucleotide in the antisense strand may be modified with Ganciclovir or Denavir. As shown in FIGS. 8A-B, one nucleotide on the antisense strand may be 3′-ocp. As shown in FIGS. 3A-3J, 4A-4AB, 5A-F, 6A-K, 7A-D, and 8A-B the ds-siNA does not contain a base pair between 2′-fluoro nucleotides on the sense and antisense strands. In some embodiments, the 2′-O-methyl, 2′-ocp, or 2′-omcp nucleotide at position 1 from the 5′ end of the sense strand is further modified to contain a 5′ stabilizing end cap. In some embodiments, the 2′-O-methyl, 2′-ocp, or 2′-omcp nucleotide at position 1 from the 5′ end of the antisense strand is further modified to contain a 5′ stabilizing end cap. In some embodiments, the 2′-O-methyl, 2′-ocp, or 2′-omcp nucleotide at position 1 from the 5′ end of the sense strand is further modified to contain a phosphorylation blocker. In some embodiments, the 2′-O-methyl, 2′-ocp, or 2′-omcp nucleotide at position 1 from the 3′ end of the sense strand is further modified to contain a phosphorylation blocker. In some embodiments, the 2′-O-methyl, 2′-ocp, or 2′-omcp nucleotide at position 1 from the 5′ end of the antisense strand is further modified to contain a phosphorylation blocker. In some embodiments, the 2′-O-methyl, 2′-ocp, or 2′-omcp nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIG. 3A); a sense strand consisting of 19 nucleotides, wherein 2′-fluoro nucleotides are at positions 3, 7, 8, and 17 from the 5′ end of the sense strand, and wherein 2′-O-methyl nucleotides are at positions 1, 2, 4-6, 9-16, 18, and 19 from the 5′ end of the sense strand (FIG. 3B); a sense strand consisting of 19 nucleotides, wherein 2′-fluoro nucleotides are at positions 3, 7-9, 12 and 17 from the 5′ end of the sense strand, and wherein 2′-O-methyl nucleotides are at positions 1, 2, 4-6, 10, 11, 13-16, 18, and 19 from the 5′ end of the sense strand (FIG. 3C); a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand (FIG. 3D-F); a sense strand consisting of 21 nucleotides, wherein 2′-fluoro nucleotides are at positions 5, 9-11, 14, and 19 from the 5′ end of the sense strand, and wherein 2′-O-methyl nucleotides are at positions 1-4, 6-8, 12, 13, 15-18, 20, and 21 from the 5′ end of the sense strand (FIG. 3G); a sense strand consisting of 21 nucleotides, wherein 2′-fluoro nucleotides are at positions 7 and 9-11 from the 5′ end of the sense strand, and wherein 2′-O-methyl nucleotides are at positions 1-6, 8, and 12-21 from the 5′ end of the sense strand (FIG. 3H); a sense strand consisting of 21 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIG. 3I); and a sense strand consisting of 21 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, Ind 19 from the 5′ end of the sense strand (FIG. 3J).

A ds-siNA may comprise an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O-methyl nucleotides (FIGS. 3A and B); an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O-methyl nucleotides (FIGs. C and D); an antisense strand consisting of 21 nucleotides, wherein the nucleotides in the antisense strand comprise an alternating 1:2 modification pattern, and wherein 1 nucleotide is a 2′-fluoro nucleotide and 2 nucleotides are 2′-O-methyl nucleotides (FIG. 3E); an antisense strand consisting of 21 nucleotides, wherein 2′-fluoro nucleotides are at positions 2, 6, 14, and 16 from the 5′ end of the antisense strand, and wherein 2′-O-methyl nucleotides are at positions 1, 3-5, 7-13, 15, and 17-21 from the 5′ end of (FIG. 3F); an antisense strand consisting of 23 nucleotides, wherein 2′-flouro nucleotides are at positions 2 and 14 from the 5′ end of the antisense strand, and wherein 2′-O-methyl nucleotides are at positions 1, 3-13, and 15-23 from the 5′ end of the antisense strand (FIG. 3G); an antisense strand consisting of 23 nucleotides, wherein 2′-flouro nucleotides are at positions 2, 6, 14, and 16 from the 5′ end of the antisense strand, and wherein 2′-O-methyl nucleotides are at positions 1, 3-5, 7-13, 15, and 17-23 from the 5′ end of the antisense strand (FIG. 3H); an antisense strand consisting of 23 nucleotides, wherein 2′-flouro nucleotides are at positions 2, 7, and 14 from the 5′ end of the antisense strand, and wherein 2′-O-methyl nucleotides are at positions 1, 3-6, 8-13, and 15-23 from the 5′ end of the antisense strand (FIG. 3I); and an antisense strand consisting of 23 nucleotides, wherein 2′-flouro nucleotides are at positions 2, 14, and 16 from the 5′ end of the antisense strand, and wherein 2′-O-methyl nucleotides are at positions 1, 3-13, 15, and 17-23 from the 5′ end of the antisense strand (FIG. 3J).

As shown in FIGS. 3A-G, I, and J, the ds-siNA may further comprise a conjugated moiety attached to the 3′ end of the sense strand. As shown in FIGS. 3A-J, the ds-siNA may further comprise phosphorothioate internucleoside linkages between the nucleotides at positions 1 and 2 and positions 2 and 3 from the 5′ end of the sense strand.

As shown in FIGs. A-F, I and J, the ds-siNA may further comprise phosphorothioate internucleoside linkages between the nucleotides at positions 1 and 2; positions 2 and 3; positions 19 and 20; and positions 20 and 21 from the 5′ end of the antisense strand. As shown in FIGs. G and H, the ds-siNA may further comprise phosphorothioate internucleoside linkages between the nucleotides at positions 1 and 2; positions 2 and 3; positions 21 and 22; and positions 22 and 23 from the 5′ end of the antisense strand.

Optionally, the nucleotides at positions 22 and 23 of from the 5′ end of the antisense strand of FIGS. 3G and H may be unlocked nucleotides. The ds-siNA may optionally comprise a vinyl phosphonate attached to the 5′ end of the antisense strand (FIG. 3H), but in some embodiments, a 5′ end cap disclosed herein may be suitable as well.

In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand (FIGS. 4A-D, P, and R-AB); a sense strand consisting of 19 nucleotides, wherein 2′-fluoro nucleotides are at positions 5 and 7-9 from the 5′ end of the sense strand, 2′-O-methyl nucleotides are at positions 2, 4, 11, 13, 15, 17, and 19 from the 5′ end of the sense strand, and wherein 2′-ocp nucleotides are at positions 1, 3, 6, 10, 12, 14, 16, and 18 from the 5′ end of the sense strand (FIGS. 4E, G-J); a sense strand consisting of 19 nucleotides, wherein 2′-fluoro nucleotides are at positions 5 and 7-9 from the 5′ end of the sense strand, 2′-O-methyl nucleotides are at positions 2, 4, 11, 13, 15, and 17 from the 5′ end of the sense strand, and wherein 2′-omcp nucleotides are at positions 1, 3, 6, 10, 12, 14, 16, 18, and 19 from the 5′ end of the sense strand (FIGS. 4F and K—N); or a sense strand consisting of 19 nucleotides, wherein 2′-fluoro nucleotides are at positions 5 and 7-9 from the 5′ end of the sense strand, and wherein 2′-omcp nucleotides are at positions 1-4, 6, and 10-19 from the 5′ end of the sense strand (FIGS. 4O and Q).

A ds-siNA may comprise an antisense strand consisting of 21 nucleotides, wherein 2′-flouro nucleotides are at positions 2, 5, 8, 14, and 17 from the 5′ end of the antisense strand, 2′-O-methyl nucleotides are at positions 3, 4, 6, 9, 11, 13, 16, 19, and 21 from the 5′ end of the antisense strand, and wherein 2′-ocp nucleotides are at positions 1, 7, 10, 12, 15, 18, and 20 from the 5′ end of the antisense strand (FIGS. 4A, G, and K); an antisense strand consisting of 21 nucleotides, wherein 2′-flouro nucleotides are at positions 2, 5, 8, 14, and 17 from the 5′ end of the antisense strand, 2′-O-methyl nucleotides are at positions 1, 3, 4, 6, 7, 9, 11, 13, 16, 19, and 21 from the 5′ end of the antisense strand, and wherein 2′-ocp nucleotides are at positions 10, 12, 15, 18, and 20 from the 5′ end of the antisense strand (FIGS. 4B, H, and L); an antisense strand consisting of 21 nucleotides, wherein 2′-flouro nucleotides are at positions 2, 5, 8, 14, and 17 from the 5′ end of the antisense strand, 2′-O-methyl nucleotides are at positions 3, 6, 9, 11, 13, 16, 19, and 21 from the 5′ end of the antisense strand, and wherein 2′-omcp nucleotides are at positions 1, 4, 7, 10, 12, 15, 18, and 20 from the 5′ end of the antisense strand (FIGS. 4C, I, and M); an antisense strand consisting of 21 nucleotides, wherein 2′-flouro nucleotides are at positions 2, 5, 8, 14, and 17 from the 5′ end of the antisense strand, 2′-O-methyl nucleotides are at positions 1, 3, 4, 6, 7, 11, 13, 16, 19, and 21 from the 5′ end of the antisense strand, and wherein 2′-omcp nucleotides are at positions 9, 10, 12, 15, 18, and 20 from the 5′ end of the antisense strand (FIGS. 4D and J); an antisense strand consisting of 21 nucleotides, wherein 2′-flouro nucleotides are at positions 2, 5, 8, 14, and 17 from the 5′ end of the antisense strand, and wherein 2′-O-methyl nucleotides are at positions 1, 3, 4, 6, 7, 9-13, 15, 16, and 18-21 from the 5′ end of the antisense strand (FIGS. 4E, F, and O); an antisense strand consisting of 21 nucleotides, wherein 2′-flouro nucleotides are at positions 2, 5, 8, 14, and 17 from the 5′ end of the antisense strand, 2′-O-methyl nucleotides are at positions 1, 3, 4, 6, 7, 11, 13, 16, 19, and 21 from the 5′ end of the antisense strand, and wherein 2′-omcp nucleotides are at positions 9, 10, 12, 15, 18, and 20 from the 5′ end of the antisense strand (FIG. 4N); an antisense strand consisting of 21 nucleotides, wherein 2′-flouro nucleotides are at positions 2, 5, 8, 14, and 17 from the 5′ end of the antisense strand, and wherein 2′-omcp nucleotides are at positions 1, 3, 4, 6, 7, 9-12, 15, 16, and 18-21 from the 5′ end of the antisense strand (FIGS. 4P and Q); an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7-13, 15, and 17-20 from the 5′ end of the antisense strand, and wherein a 2′-ocp nucleotide is at position 21 from the 5′ end of the antisense strand (FIG. 4R); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-21 from the 5′ end of the antisense strand, and wherein a 2′-ocp nucleotide is at positions 1 from the 5′ end of the antisense strand (FIG. 4S); an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7-13, 15, and 17-20 from the 5′ end of the antisense strand, and wherein a 2′-omcp nucleotides is at positions 21 from the 5′ end of the antisense strand (FIG. 4T); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-21 from the 5′ end of the antisense strand, and wherein a 2′-omcp nucleotide is at position 1 from the 5′ end of the antisense strand (FIG. 4U); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-20 from the 5′ end of the antisense strand, and wherein 2′-ocp nucleotides are at positions 1 and 21 from the 5′ end of the antisense strand (FIG. 4V); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-20 from the 5′ end of the antisense strand, and wherein 2′-omcp nucleotides are at positions 1 and 21 from the 5′ end of the antisense strand (FIG. 4W); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-19 from the 5′ end of the antisense strand, and wherein 2′-ocp nucleotides are at positions 1, 20, and 21 from the 5′ end of the antisense strand (FIG. 4X); an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7, 8, 10, 12, 18, and 20 from the 5′ end of the antisense strand, and wherein 2′-ocp nucleotides are at positions 9, 11, 13, 15, 17, 19, and 21 from the 5′ end of the antisense strand (FIG. 4Y); an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7, 8, 10, 12, 18, and 20 from the 5′ end of the antisense strand, and wherein 2′-omcp nucleotides are at positions 9, 11, 13, 15, 17, 19, and 21 from the 5′ end of the antisense strand (FIG. 4Z); an antisense strand consisting of 21 nucleotides, wherein 2′-flouro nucleotides are at positions 2, 6, 14, and 16 from the 5′ end of the antisense strand, wherein 2′-O-methyl nucleotides are at positions 4, 8, 10, 12, 18, and 20 from the 5′ end of the antisense strand, and wherein 2′-ocp nucleotides are at positions 1, 3, 5, 7, 9, 11, 13, 15, 17, 19 and 21 from the 5′ end of the antisense strand (FIG. 4AA); or an antisense strand consisting of 21 nucleotides, wherein 2′-flouro nucleotides are at positions 2, 6, 14, and 16 from the 5′ end of the antisense strand, wherein 2′-O-methyl nucleotides are at positions 4, 8, 10, 12, 18, and 20 from the 5′ end of the antisense strand, and wherein 2′-omcp nucleotides are at positions 1, 3, 5, 7, 9, 11, 13, 15, 17, 19 and 21 from the 5′ end of the antisense strand (FIG. 4AB).

Optionally, the ds-siNA may further comprise a conjugated moiety attached to the 3′ end of the sense strand. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 19 and 20, and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-ocp nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker.

In some embodiments, the 2′-ocp nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand (FIGS. 5A-C, E, and F); or a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIG. 5D).

A ds-siNA may comprise: an antisense strand consisting of 21 nucleotides, wherein 2′-flouro nucleotides are at positions 2, 5, 8, 14, and 17 from the 5′ end of the antisense strand, wherein 2′-O-methyl nucleotides are at positions 3, 4, 6, 7, 9-13, 15, 16, and 18-21 from the 5′ end of the antisense strand, and wherein a vmX nucleotide is at positions 1 from the 5′ end of the antisense strand (FIGS. 5A, E, and F); an antisense strand consisting of 21 nucleotides, wherein 2′-flouro nucleotides are at positions 2, 5, 8, 14, and 17 from the 5′ end of the antisense strand, wherein 2′-O-methyl nucleotides are at positions 3, 4, 6, 7, 9-13, 15, 16, and 18-20 from the 5′ end of the antisense strand, wherein a 2′-ocp nucleotide is at position 21 from the 5′ end of the antisense strand, and wherein a vmX nucleotide is at position 1 from the 5′ end of the antisense strand (FIG. 5b); an antisense strand consisting of 21 nucleotides, wherein 2′-flouro nucleotides are at positions 2, 5, 8, 14, and 17 from the 5′ end of the antisense strand, wherein 2′-O-methyl nucleotides are at positions 3, 4, 6, 7, 9-13, 15, 16, and 18-20 from the 5′ end of the antisense strand, wherein a 2′-omcp nucleotide is at position 21 from the 5′ end of the antisense strand, and wherein a vmX nucleotide is at position 1 from the 5′ end of the antisense strand (FIG. 5C); or an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 8-13, and 15-20 from the 5′ end of the antisense strand, and wherein a 2′-ocp nucleotide is at position 21 from the 5′ end of the antisense strand (FIG. 5D).

Optionally, the ds-siNA may further comprise a conjugated moiety attached to the 3′ end of the sense strand. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 19 and 20, and positions 20 and 21 from the 5′ end of the antisense strand. In some embodiments, phosphorothioate internucleoside linkages may be an S enantiomer. In some embodiments, the phosphorothioate internucleoside linkages may be an R enantiomer.

In some embodiments, the 2′-O-methyl nucleotide at position 1 from the 5′ end of the sense strand is further modified to contain a 5′ stabilizing end cap. In some embodiments, the vmX nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the vmX nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIGS. 6A-G); or a sense strand consisting of 19 nucleotides, wherein 2′-fluoro nucleotides are at positions 5 and 7-9, 12 from the 5′ end of the sense strand, and wherein 2′-O-methyl nucleotides are at positions 1-4, 6, and 10-19 from the 5′ end of the sense strand (FIGS. 6H-K).

A ds-siNA may comprise: an antisense strand consisting of 21 nucleotides, wherein 2′-flouro nucleotides are at positions 2, 7, and 14 from the 5′ end of the antisense strand, wherein 2′-O-methyl nucleotides are at positions 1, 3, 4, 6, 8-13, and 15-21 from the 5′ end of the antisense strand, and wherein a xylo nucleotide is at position 5 from the 5′ end of the antisense strand (FIG. 6A); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-5, 8-13, and 15-21 from the 5′ end of the antisense strand, and wherein a xylo nucleotide is at position 6 from the 5′ end of the antisense strand (FIG. 6B); an antisense strand consisting of 21 nucleotides, wherein 2′-flouro nucleotides are at positions 2 and 14 from the 5′ end of the antisense strand, wherein 2′-O-methyl nucleotides are at positions 1, 3-6, 8-13, and 15-21 from the 5′ end of the antisense strand, and wherein a xylo nucleotide is at position 7 from the 5′ end of the antisense strand (FIG. 6C); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 9-13, and 15-21 from the 5′ end of the antisense strand, and wherein a xylo nucleotide is at position 8 from the 5′ end of the antisense strand (FIG. 6D); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 8-13, 15-19, and 21 from the 5′ end of the antisense strand, and wherein a xylo nucleotide is at position 20 from the 5′ end of the antisense strand (FIG. 6E); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-5, 8-13, 15-19, and 21 from the 5′ end of the antisense strand, and wherein xylo nucleotides are at positions 6 and from the 5′ end of the antisense strand (FIG. 6F); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 9-13, 15-19, and 21 from the 5′ end of the antisense strand, and wherein xylo nucleotides are at positions 8 and 20 from the 5′ end of the antisense strand (FIG. 6G); an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7-13, 15, and 17-20 from the 5′ end of the antisense strand, and wherein a xylo nucleotide is at position 21 from the 5′ end of the antisense strand (FIG. 6H); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-21 from the 5′ end of the antisense strand, and wherein a xylo nucleotide is at position 1 from the 5′ end of the antisense strand (FIG. 6I); an antisense strand consisting of 21 nucleotides, wherein 2′-flouro nucleotides are at positions 2, 6, 14, and 16 from the 5′ end of the antisense strand, wherein 2′-O-methyl nucleotides are at positions 3-5, 7-13, 15, and 17-20 from the 5′ end of the antisense strand, and wherein xylo nucleotides are at positions 1 and 21 from the 5′ end of the antisense strand (FIG. 6J); or an antisense strand consisting of 21 nucleotides, wherein 2′-flouro nucleotides are at positions 2, 6, 14, and 16 from the 5′ end of the antisense strand, wherein 2′-O-methyl nucleotides are at positions 1, 3-5, 7-13, 15, 17-19, and 21 from the 5′ end of the antisense strand, and wherein a xylo nucleotide is at position 20 from the 5′ end of the antisense strand (FIG. 6K).

In some embodiments, the xylo nucleotide can be a 2′-OMe-3′-xylo nucleotide. In some embodiments, the xylo nucleotide can be a 2′-F-3′-xylo nucleotide. Optionally, the ds-siNA may further comprise a conjugated moiety attached to the 3′ end of the sense strand. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 19 and 20, and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIGS. 7A-D).

A ds-siNA may comprise: an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-5, 8-13, and 15-21 from the 5′ end of the antisense strand, and wherein an acyclic ganciclovir nucleotide analogue is at position 6 from the 5′ end of the antisense strand (FIG. 7A); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-5, 8-13, and 15-21 from the 5′ end of the antisense strand, and wherein a denavir nucleotide analogue is at position 6 from the 5′ end of the antisense strand (FIG. 7B); an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 9-13, and 15-21 from the 5′ end of the antisense strand, and wherein a ganciclovir nucleotide is at position 8 from the 5′ end of the antisense strand (FIG. 7C); or an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 9-13, and 15-21 from the 5′ end of the antisense strand, and wherein a denavir nucleotide is at position 8 from the 5′ end of the antisense strand (FIG. 7D).

In some embodiments, the acyclic ganciclovir nucleotide analogue is an S enantiomer. In some embodiments, the acyclic ganciclovir nucleotide analogue is an R enantiomer. In some embodiments, the denavir nucleotide is an S antiomer. In some embodiments, the denavir nucleotide is an R antiomer.

Optionally, the ds-siNA may further comprise a conjugated moiety attached to the 3′ end of the sense strand. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 19 and 20, and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

A ds-siNA may comprise: a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand (FIGS. 8A and B).

A ds-siNA may comprise: an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-5, 8-13, and 15-21 from the 5′ end of the antisense strand, and wherein a denavir 3′-ocp nucleotide is at position 6 from the 5′ end of the antisense strand (FIG. 8A); or an antisense strand consisting of 21 nucleotides, wherein 2′-flouro nucleotides are at positions 2, 7, and 14 from the 5′ end of the antisense strand, wherein 2′-O-methyl nucleotides are at positions 1, 3-6, 9-13, and 15-21 from the 5′ end of the antisense strand, and wherein a 3′-ocp nucleotide is at position 8 from the 5′ end of the antisense strand (FIG. 8B).

Optionally, the ds-siNA may further comprise a conjugated moiety attached to the 3′ end of the sense strand. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 19 and 20, and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

In some embodiments, the nucleotide at position 1 from the 5′ end of the sense strand is a 5′vinyl phosphonate dimer moiety (e.g., either enantiomer of PP20 or PP2OH), a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-munb nucleotide, a d2vm nucleotide, or a d2vmA nucleotide, a d2vd3U nucleotide, an omeco-d3U nucleotide, a 4hU nucleotide, a v-mun nucleotide, a c2o-4h nucleotide, an omeco-munb nucleotide, or a d2vmA nucleotide. In some embodiments, the nucleotide at position 1 from the 5′ end of the antisense strand is a 5′vinyl phosphonate dimer moiety (e.g., either enantiomer of PP20 or PP2OH), a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-munb nucleotide, a d2vm nucleotide, or a d2vmA nucleotide. In some embodiments, the nucleotide at position 1 from the 3′ end of the sense strand is a 5′vinyl phosphonate dimer moiety (e.g., either enantiomer of PP20 or PP2OH), a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-munb nucleotide, a d2vm nucleotide, or a d2vmA nucleotide. In some embodiments, the nucleotide at position 1 from the 3′ end of the antisense strand is a 5′vinyl phosphonate dimer moiety (e.g., either enantiomer of PP20 or PP2OH), a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-munb nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a 3′,4′-secoF, 3′,4′-secoFA, fB, fN, f(4nh) Q, f4P, f2P, or fX nucleotide. In some embodiments, at least 1, 2, 3, 4 or more 2′-fluoro nucleotides on the antisense strand is a 3′,4′-secoF, 3′,4′-secoFA, fB, fN, f(4nh) Q, f4P, f2P, or fX nucleotide. In some embodiments, at least 1, 2, 3, 4 or more 2′-O-methyl nucleotides on the sense or antisense strand is a 2′-O-methyl nucleotide mimic. In some embodiments, at least 1, 2, 3, 4 or more nucleotides on the sense strand or antisense strand is a 2′-ocp, 2′-ocmp, 3′-ocp, 3′-omcp, 5cp, 5mcp, mun12, moe, 3m, L-2′-OMe, tn20, tn, 2′-OMe-3′-xylo, or 2′-F-3′-xylo nucleotide. In some embodiments, one or more nucleotides in the sense strand and/or the antisense strand may be a 3′,4′-seco modified nucleotide in which the bond between the 3′ and 4′ positions of the furanose ring is broken (e.g., 3′4′-secoOBz, 3′4′-secoF, or mun34).

In some embodiments, the sense strand and/or the antisense strand may also comprise one or more nucleotide analogs (e.g., An1 and An2).

siNA Sense Strand

Any of the siNA molecules described herein may comprise a sense strand. The sense strand may comprise a first nucleotide sequence. The first nucleotide sequence may be 15 to 30, 15 to 25, 15 to 23, 17 to 23, 19 to 23, or 19 to 21 nucleotides in length. In some embodiments, the first nucleotide sequence is 15, 16, 17, 18, 19, 20, 21, 22, 23, 24, 25, 26, 27, 28, 29, or 30 nucleotides in length. In some embodiments, the first nucleotide sequence is at least 19 nucleotides in length. In some embodiments, the first nucleotide sequence is at least 21 nucleotides in length.

In some embodiments, the sense strand is the same length as the first nucleotide sequence. In some embodiments, the sense strand is longer than the first nucleotide sequence. In some embodiments, the sense strand may further comprise 1, 2, 3, 4, or 5 or more nucleotides than the first nucleotide sequence. In some embodiments, the sense strand may further comprise a deoxyribonucleic acid (DNA). In some embodiments, the DNA is thymine (T). In some embodiments, the sense strand may further comprise a TT sequence. In some embodiments, the sense strand may further comprise one or more modified nucleotides that are adjacent to the first nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, 2′-ocp nucleotide, 2′-omcp nucleotide, or a nucleotide comprising a modified nucleobase).

In some embodiments, the first nucleotide sequence comprises 15, 16, 17, 18, 19, 20, 21, 22, 23, or more modified nucleotides independently selected from a 2′-O-methyl nucleotide, a 2′-fluoro nucleotide, a 2′-ocp nucleotide, a 2′-omcp nucleotide, a 3′-ocp nucleotide, a 2′-OMe-3′-xylo nucleotide, a 2′-F-3′-xylo nucleotide, a vmX nucleotide, a ganciclovir nucleotide (referred to interchangeably as “acyclic ganciclovir nucleotide analogue” herein), and a denavir nucleotide. In some embodiments, 70%, 75%, 80%, 85%, 90%, 95% or 100% of the nucleotides in the first nucleotide sequence are modified nucleotides independently selected from a 2′-O-methyl nucleotide, a 2′-fluoro nucleotide, a 2′-ocp nucleotide, a 2′-omcp nucleotide, a 3′-ocp nucleotide, a 2′-OMe-3′-xylo nucleotide, a 2′-F-3′-xylo nucleotide, a vmX nucleotide, a ganciclovir nucleotide, and a denavir nucleotide. In some embodiments, 100% of the nucleotides in the first nucleotide sequence are modified nucleotides independently selected from a 2′-O-methyl nucleotide, a 2′-fluoro nucleotide, a 2′-ocp nucleotide, a 2′-omcp nucleotide, a 3′-ocp nucleotide, a 2′-OMe-3′-xylo nucleotide, a 2′-F-3′-xylo nucleotide, a vmX nucleotide, a ganciclovir nucleotide, and a denavir nucleotide. In some embodiments, the 2′-O-methyl nucleotide is a 2′-O-methyl nucleotide mimic. In some embodiments, the 2′-fluoro nucleotide is a 2′-fluoro nucleotide mimic.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the first nucleotide sequence are 2′-O-methyl nucleotides. In some embodiments, 1 or none of the modified nucleotides of the first nucleotide sequence are 2′-O-methyl nucleotides. In some embodiments, between about 2 to 20 modified nucleotides of the first nucleotide sequence are 2′-O-methyl nucleotides. In some embodiments, between about 5 to 25 modified nucleotides of the first nucleotide sequence are 2′-O-methyl nucleotides. In some embodiments, between about 10 to 25 modified nucleotides of the first nucleotide sequence are 2′-O-methyl nucleotides. In some embodiments, between about 12 to 25 modified nucleotides of the first nucleotide sequence are 2′-O-methyl nucleotides. In some embodiments, at least about 10, 11, 12, 13, 14, 15, 16, 17, 18, 19, 20, 21, or 22 modified nucleotides of the first nucleotide sequence are 2′-O-methyl nucleotides. In some embodiments, at least about 12 modified nucleotides of the first nucleotide sequence are 2′-O-methyl nucleotides. In some embodiments, at least about 13 modified nucleotides of the first nucleotide sequence are 2′-O-methyl nucleotides. In some embodiments, at least about 14 modified nucleotides of the first nucleotide sequence are 2′-O-methyl nucleotides. In some embodiments, at least about 15 modified nucleotides of the first nucleotide sequence are 2′-O-methyl nucleotides. In some embodiments, at least about 16 modified nucleotides of the first nucleotide sequence are 2′-O-methyl nucleotides. In some embodiments, at least about 17 modified nucleotides of the first nucleotide sequence are 2′-O-methyl nucleotides. In some embodiments, at least about 18 modified nucleotides of the first nucleotide sequence are 2′-O-methyl nucleotides. In some embodiments, at least about 19 modified nucleotides of the first nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides. In some embodiments, less than or equal to 21 modified nucleotides of the first nucleotide sequence are 2′-O-methyl nucleotides. In some embodiments, less than or equal to 20 modified nucleotides of the first nucleotide sequence are 2′-O-methyl nucleotides. In some embodiments, less than or equal to 19 modified nucleotides of the first nucleotide sequence are 2′-O-methyl nucleotides. In some embodiments, less than or equal to 18 modified nucleotides of the first nucleotide sequence are 2′-O-methyl nucleotides. In some embodiments, less than or equal to 17 modified nucleotides of the first nucleotide sequence are 2′-O-methyl nucleotides. In some embodiments, less than or equal to 16 modified nucleotides of the first nucleotide sequence are 2′-O-methyl nucleotides. In some embodiments, less than or equal to 15 modified nucleotides of the first nucleotide sequence are 2′-O-methyl nucleotides. In some embodiments, less than or equal to 14 modified nucleotides of the first nucleotide sequence are 2′-O-methyl nucleotides. In some embodiments, less than or equal to 13 modified nucleotides of the first nucleotide sequence are 2′-O-methyl nucleotides. In some embodiments, at least one modified nucleotide of the first nucleotide sequence is a 2′-O-methyl pyrimidine. In some embodiments, at least 5, 6, 7, 8, 9, or 10 modified nucleotides of the first nucleotide sequence are 2′-O-methyl pyrimidines. In some embodiments, at least one modified nucleotide of the first nucleotide sequence is a 2′-O-methyl purine. In some embodiments, at least 5, 6, 7, 8, 9, or 10 modified nucleotides of the first nucleotide sequence are 2′-O-methyl purines. In some embodiments, the 2′-O-methyl nucleotide is a 2′-O-methyl nucleotide mimic.

In some embodiments, between 2 to 15 modified nucleotides of the first nucleotide sequence are 2′-fluoro nucleotides. In some embodiments, between 2 to 10 modified nucleotides of the first nucleotide sequence are 2′-fluoro nucleotides. In some embodiments, between 2 to 6 modified nucleotides of the first nucleotide sequence are 2′-fluoro nucleotides. In some embodiments, 1 to 6, 1 to 5, 1 to 4, or 1 to 3 modified nucleotides of the first nucleotide sequence are 2′-fluoro nucleotides. In some embodiments, at least 1, 2, 3, 4, 5, or 6 modified nucleotides of the first nucleotide sequence are 2′-fluoro nucleotides. In some embodiments, at least 1 modified nucleotide of the first nucleotide sequence is a 2′-fluoro nucleotide. In some embodiments, at least 2 modified nucleotides of the first nucleotide sequence are 2′-fluoro nucleotides. In some embodiments, at least 3 modified nucleotides of the first nucleotide sequence are 2′-fluoro nucleotides. In some embodiments, at least 4 modified nucleotides of the first nucleotide sequence are 2′-fluoro nucleotides. In some embodiments, at least 5 modified nucleotides of the first nucleotide sequence are 2′-fluoro nucleotides. In some embodiments, at least 6 modified nucleotides of the first nucleotide sequence are 2′-fluoro nucleotides. In some embodiments, 10, 9, 8, 7, 6, 5, 4, 3 or fewer modified nucleotides of the first nucleotide sequence are 2′-fluoro nucleotides. In some embodiments, 10 or fewer modified nucleotides of the first nucleotide sequence are 2′-fluoro nucleotides. In some embodiments, 7 or fewer modified nucleotides of the first nucleotide sequence are 2′-fluoro nucleotides. In some embodiments, 6 or fewer modified nucleotides of the first nucleotide sequence are 2′-fluoro nucleotides. In some embodiments, 5 or fewer modified nucleotides of the first nucleotide sequence are 2′-fluoro nucleotides. In some embodiments, 4 or fewer modified nucleotides of the first nucleotide sequence are 2′-fluoro nucleotides. In some embodiments, 3 or fewer modified nucleotides of the first nucleotide sequence are 2′-fluoro nucleotides. In some embodiments, 2 or fewer modified nucleotides of the first nucleotide sequence are 2′-fluoro nucleotides. In some embodiments, at least one modified nucleotide of the first nucleotide sequence is a 2′-fluoro pyrimidine. In some embodiments, 1, 2, 3, 4, 5, or 6 modified nucleotides of the first nucleotide sequence are 2′-fluoro pyrimidines. In some embodiments, at least one modified nucleotide of the first nucleotide sequence is a 2′-fluoro purine. In some embodiments, 1, 2, 3, 4, 5, or 6 modified nucleotides of the first nucleotide sequence are 2′-fluoro purines. In some embodiments, the 2′-fluoro nucleotide is a 2′-fluoro nucleotide mimic.

In some embodiments, the nucleotide at position 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, at least four nucleotides at positions 3, 5, 7, 8, 9, 10, 11, 12, 14, 17, and/or 19 from the 5′ end of the first nucleotide sequence are 2′-fluoro nucleotides. In some embodiments, at least five nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 3, 5, 7, 8, 9, 10, 11, 12, 14, 17, and/or 19 from the 5′ end of the first nucleotide sequence is a 2′-fluoro nucleotide. In some embodiments, the nucleotide at positions 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 5 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 10 from the 5′ end of the first nucleotide sequence is a 2′-fluoro nucleotide. In some embodiments, the nucleotide at position 11 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 14 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the nucleotide at position 19 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 3, 7, 8, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 5, 7, 8, and/or 9 from the 5′ end of the first nucleotide sequence is a 2′-fluoro nucleotide. In some embodiments, the nucleotide at position 5, 9, 10, 11, 12, and/or 19 from the 5′ end of the first nucleotide sequence is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

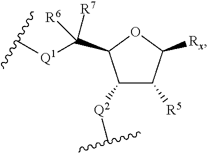

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analog or derivative thereof.

In some embodiments, the 2′-fluoro or 2′-O-methyl nucleotide mimic is a nucleotide mimic of Formula (16)-Formula (20):

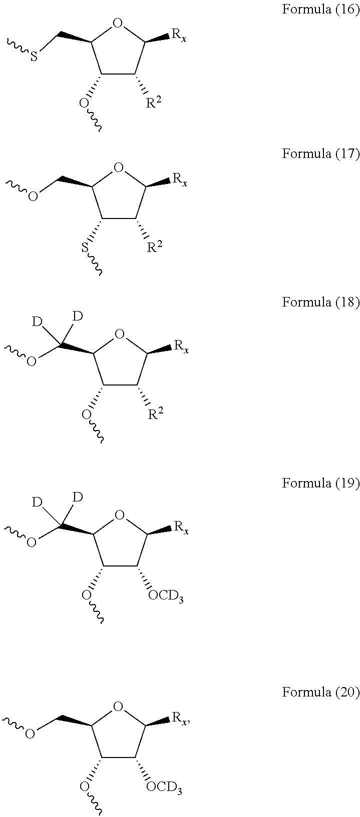

wherein Rₓ is independently a nucleobase, aryl, heteroaryl, or H and R² is F or —OCH₃. In some embodiments, the nucleobase is selected from thymine, cytosine, guanine, adenine, uracil, and an analog or derivative thereof.

In some embodiments, the sense strand, the antisense strand, or both may each independently comprise at least 1, at least 2, at least 3, at least 4, or at least 5 or more modified nucleotide(s) and/or nucleotide analog(s), having the following chemical structure:

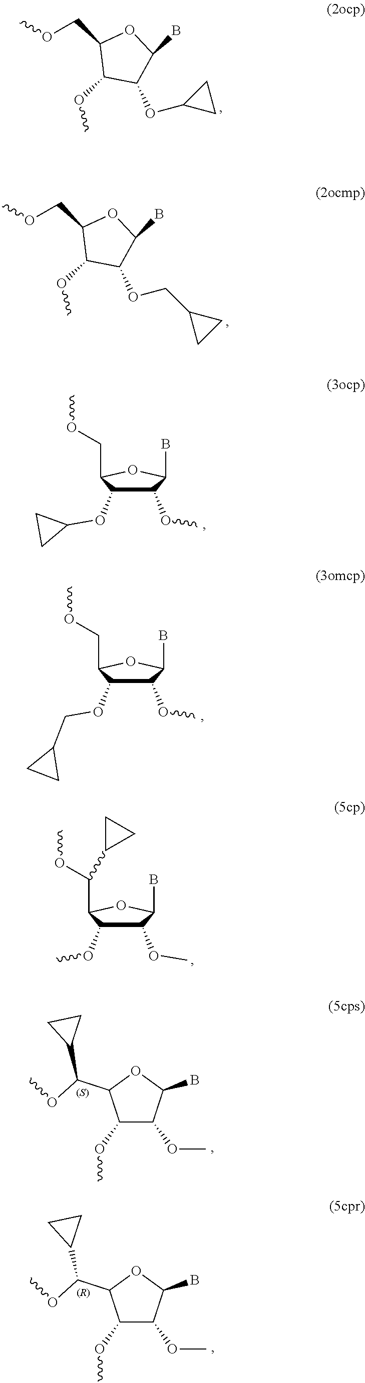

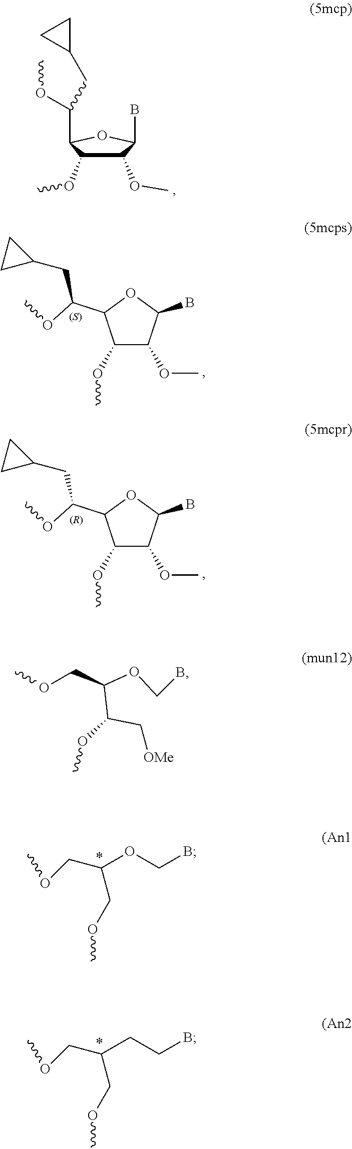

wherein * represents a chiral center),

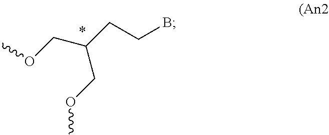

wherein * represents a chiral center),

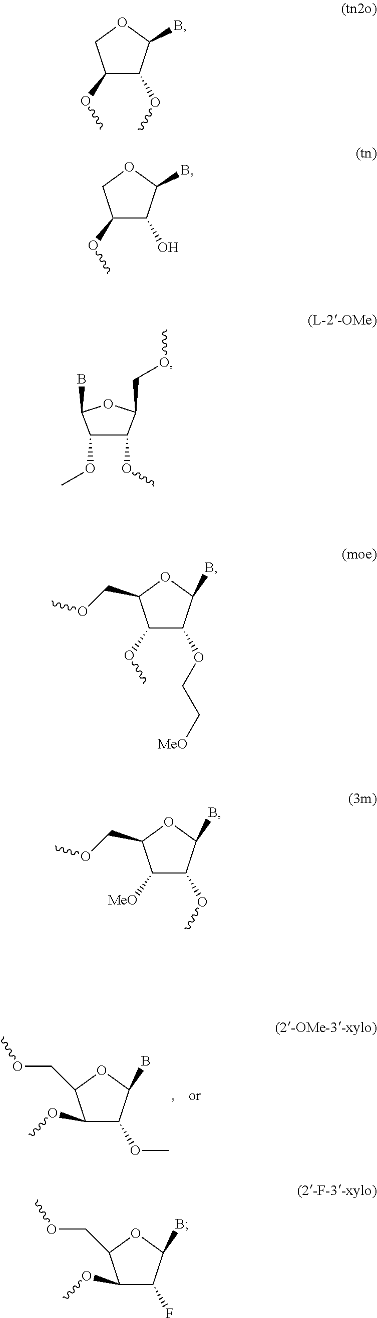

wherein B is a nucleobase, aryl, heteroaryl, or H, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

In some embodiments, the sense strand, the antisense strand, or both may each additionally independently comprise at least 1, at least 2, at least 3, at least 4, or at least 5 or more modified nucleotide(s) having the following chemical structure:

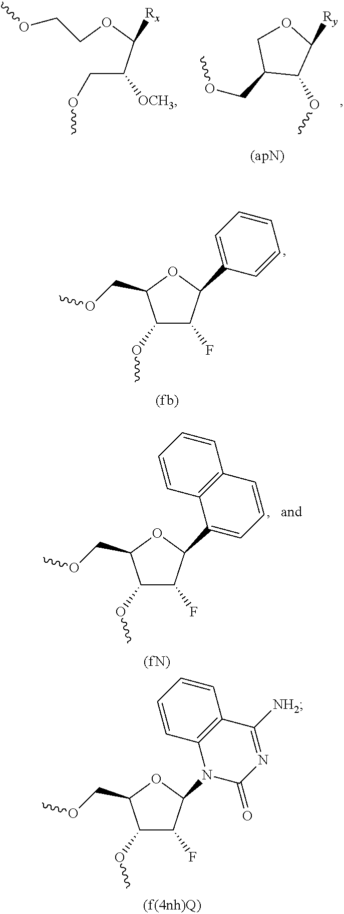

wherein Rₓ is a nucleobase, aryl, heteroaryl, or H and R_{y} is a nucleobase, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

In some embodiments, the sense strand, the antisense strand, or both may each additionally independently comprise at least 1, at least 2, at least 3, at least 4, or at least 5 or more modified nucleotide(s) having the following chemical structure:

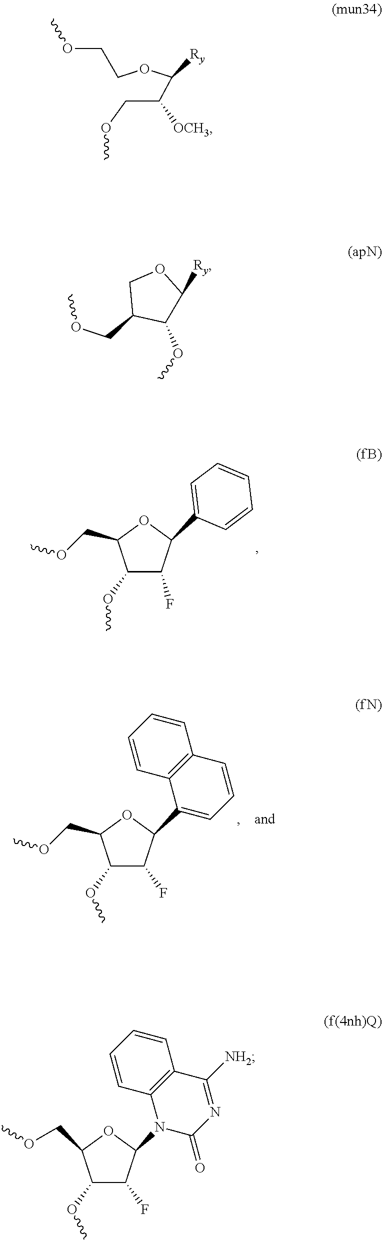

wherein R_{y} is a nucleobase, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

For the purposes of the present disclosure, the modified nucleotide or nucleotide analog may be in any position of the sense strand. In some embodiments, the modified nucleotide or nucleotide analog may be at position 1, 2, 3, 4, 5, 6, 7, 8, 9, 10, 11, 12, 13, 14, 15, 16, 17, 18, 19, 20, 21, 22, or 23 of the sense strand relative to the 5′ end.

In some embodiments, the first nucleotide sequence comprises, consists of, or consists essentially of ribonucleic acids (RNAs). In some embodiments, the first nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the first nucleotide sequence are independently selected from 2′-O-methyl RNA and 2′-fluoro RNA.

In some embodiments, the sense strand may further comprise one or more internucleoside linkages independently selected from a phosphodiester (PO) internucleoside linkage, phosphorothioate (PS) internucleoside linkage, mesyl phosphoramidate internucleoside linkage (Ms), phosphorodithioate internucleoside linkage, and PS-mimic internucleoside linkage. In some embodiments, the PS-mimic internucleoside linkage is a sulfo internucleoside linkage.

In some embodiments, the sense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of the first nucleotide sequence. In some embodiments, at least one phosphorothioate internucleoside linkage is between the nucleotides at positions 2 and 3 from the 5′ end of the first nucleotide sequence. In some embodiments, the sense strand comprises two phosphorothioate internucleoside linkages between the nucleotides at positions 1 to 3 from the 5′ end of the first nucleotide sequence.

In some embodiments, the sense strand may further comprise at least 1, 2, 3, 4, 5, 6, 7, 8, 9, 10, 11, 12, 13, 14, or 15 or more mesyl phosphoramidate internucleoside linkages. In some embodiments, the sense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the sense strand comprises 2 to 10, 2 to 8, 2 to 6, 1 to 5, 1 to 4, 1 to 3, or 1 to 2 mesyl phosphoramidate internucleoside linkages. In some embodiments, the sense strand comprises 1 to 2 mesyl phosphoramidate internucleoside linkages. In some embodiments, the sense strand comprises 2 to 4 mesyl phosphoramidate internucleoside linkages.

In some embodiments, the sense strand may comprise any of the modified nucleotides disclosed in the sub-section titled “Modified Nucleotides” below. In some embodiments, the sense strand may comprise a 5′-stabilized end cap, and the 5′-stabilized end cap may be selected from those disclosed in the sub-section titled “5′-Stabilized End Cap” below.

siNA Antisense Strand

Any of the siNA molecules described herein may comprise an antisense strand. The antisense strand may comprise a second nucleotide sequence. The second nucleotide sequence may be 15 to 30, 15 to 25, 15 to 23, 17 to 23, 19 to 23, or 19 to 21 nucleotides in length. In some embodiments, the second nucleotide sequence is 15, 16, 17, 18, 19, 20, 21, 22, 23, 24, 25, 26, 27, 28, 29, or 30 nucleotides in length. In some embodiments, the second nucleotide sequence is at least 19 nucleotides in length. In some embodiments, the second nucleotide sequence is at least 21 nucleotides in length.

In some embodiments, the antisense strand is the same length as the second nucleotide sequence. In some embodiments, the antisense strand is longer than the second nucleotide sequence. In some embodiments, the antisense strand may further comprise 1, 2, 3, 4, or 5 or more nucleotides than the second nucleotide sequence. In some embodiments, the antisense strand is the same length as the sense strand. In some embodiments, the antisense strand is longer than the sense strand. In some embodiments, the antisense strand may further comprise 1, 2, 3, 4, or 5 or more nucleotides than the sense strand. In some embodiments, the antisense strand may further comprise a deoxyribonucleic acid (DNA). In some embodiments, the DNA is thymine (T). In some embodiments, the antisense strand may further comprise a TT sequence. In some embodiments, the antisense strand may further comprise one or more modified nucleotides that are adjacent to the second nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, a 2′-ocp nucleotide, a 2′-omcp nucleotide, 3′-ocp nucleotide, 2′-OMe-3′-xylo nucleotide, 2′-F-3′-xylo nucleotide, vmX nucleotide, ganciclovir nucleotide, and a denavir nucleotide or a nucleotide comprising a modified nucleobase).

In some embodiments, the second nucleotide sequence comprises 15, 16, 17, 18, 19, 20, 21, 22, 23, or more modified nucleotides independently selected from a 2′-O-methyl nucleotide, a 2′-fluoro nucleotide, a 2′-ocp nucleotide, and a 2′-omcp nucleotide. In some embodiments, 70%, 75%, 80%, 85%, 90%, 95% or 100% of the nucleotides in the second nucleotide sequence are modified nucleotides independently selected from a 2′-O-methyl nucleotide, a 2′-fluoro nucleotide, a 2′-ocp nucleotide, a 2′-omcp nucleotide, a 3′-ocp nucleotide, a 2′-OMe-3′-xylo nucleotide, a 2′-F-3′-xylo nucleotide, a vinX nucleotide, a ganciclovir nucleotide, and a denavir nucleotide. In some embodiments, 100% of the nucleotides in the second nucleotide sequence are modified nucleotides independently selected from a 2′-O-methyl nucleotide, a 2′-fluoro nucleotide, a 2′-ocp nucleotide, a 2′-omcp nucleotide, a3′-ocp nucleotide, a 2′-OMe-3′-xylo nucleotide, a 2′-F-3′-xylo nucleotide, a vmX nucleotide, a ganciclovir nucleotide, and a denavir nucleotide.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the second nucleotide sequence are 2′-O-methyl nucleotides. In some embodiments, 1 or none of the modified nucleotides of the second nucleotide sequence are 2′-O-methyl nucleotides. In some embodiments, between about 2 to 20 modified nucleotides of the second nucleotide sequence are 2′-O-methyl nucleotides. In some embodiments, between about 5 to 25 modified nucleotides of the second nucleotide sequence are 2′-O-methyl nucleotides. In some embodiments, between about 10 to 25 modified nucleotides of the second nucleotide sequence are 2′-O-methyl nucleotides. In some embodiments, between about 12 to 25 modified nucleotides of the second nucleotide sequence are 2′-O-methyl nucleotides. In some embodiments, at least 10, 11, 12, 13, 14, 15, 16, 17, 18, 19, 20, 21, or 22 modified nucleotides of the second nucleotide sequence are 2′-O-methyl nucleotides. In some embodiments, at least about 12 modified nucleotides of the second nucleotide sequence are 2′-O-methyl nucleotides. In some embodiments, at least about 13 modified nucleotides of the second nucleotide sequence are 2′-O-methyl nucleotides. In some embodiments, at least about 14 modified nucleotides of the second nucleotide sequence are 2′-O-methyl nucleotides. In some embodiments, at least about 15 modified nucleotides of the second nucleotide sequence are 2′-O-methyl nucleotides. In some embodiments, at least about 16 modified nucleotides of the second nucleotide sequence are 2′-O-methyl nucleotides. In some embodiments, at least about 17 modified nucleotides of the second nucleotide sequence are 2′-O-methyl nucleotides. In some embodiments, at least about 18 modified nucleotides of the second nucleotide sequence are 2′-O-methyl nucleotides. In some embodiments, at least about 19 modified nucleotides of the second nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides. In some embodiments, less than or equal to 21 modified nucleotides of the second nucleotide sequence are 2′-O-methyl nucleotides. In some embodiments, less than or equal to 20 modified nucleotides of the second nucleotide sequence are 2′-O-methyl nucleotides. In some embodiments, less than or equal to 19 modified nucleotides of the second nucleotide sequence are 2′-O-methyl nucleotides. In some embodiments, less than or equal to 18 modified nucleotides of the second nucleotide sequence are 2′-O-methyl nucleotides. In some embodiments, less than or equal to 17 modified nucleotides of the second nucleotide sequence are 2′-O-methyl nucleotides. In some embodiments, less than or equal to 16 modified nucleotides of the second nucleotide sequence are 2′-O-methyl nucleotides. In some embodiments, less than or equal to 15 modified nucleotides of the second nucleotide sequence are 2′-O-methyl nucleotides. In some embodiments, less than or equal to 14 modified nucleotides of the second nucleotide sequence are 2′-O-methyl nucleotides. In some embodiments, less than or equal to 13 modified nucleotides of the second nucleotide sequence are 2′-O-methyl nucleotides. In some embodiments, at least one modified nucleotide of the second nucleotide sequence is a 2′-O-methyl pyrimidine. In some embodiments, at least 5, 6, 7, 8, 9, or 10 modified nucleotides of the second nucleotide sequence are 2′-O-methyl pyrimidines. In some embodiments, at least one modified nucleotide of the second nucleotide sequence is a 2′-O-methyl purine. In some embodiments, at least 5, 6, 7, 8, 9, or 10 modified nucleotides of the second nucleotide sequence are 2′-O-methyl purines. In some embodiments, the 2′-O-methyl nucleotide is a 2′-O-methyl nucleotide mimic.

In some embodiments, between 2 to 15 modified nucleotides of the second nucleotide sequence are 2′-fluoro nucleotides. In some embodiments, between 2 to 10 modified nucleotides of the second nucleotide sequence are 2′-fluoro nucleotides. In some embodiments, between 2 to 6 modified nucleotides of the second nucleotide sequence are 2′-fluoro nucleotides. In some embodiments, 1 to 6, 1 to 5, 1 to 4, or 1 to 3 modified nucleotides of the second nucleotide sequence are 2′-fluoro nucleotides. In some embodiments, at least 1, 2, 3, 4, 5, or 6 modified nucleotides of the second nucleotide sequence are 2′-fluoro nucleotides. In some embodiments, at least 1 modified nucleotide of the second nucleotide sequence is a 2′-fluoro nucleotide. In some embodiments, at least 2 modified nucleotides of the second nucleotide sequence are 2′-fluoro nucleotides. In some embodiments, at least 3 modified nucleotides of the second nucleotide sequence are 2′-fluoro nucleotides. In some embodiments, at least 4 modified nucleotides of the second nucleotide sequence are 2′-fluoro nucleotides. In some embodiments, at least 5 modified nucleotides of the second nucleotide sequence are 2′-fluoro nucleotides. In some embodiments, 10, 9, 8, 7, 6, 5, 4, 3 or fewer modified nucleotides of the second nucleotide sequence are 2′-fluoro nucleotides. In some embodiments, 10 or fewer modified nucleotides of the second nucleotide sequence are 2′-fluoro nucleotides. In some embodiments, 7 or fewer modified nucleotides of the second nucleotide sequence are 2′-fluoro nucleotides. In some embodiments, 6 or fewer modified nucleotides of the second nucleotide sequence are 2′-fluoro nucleotides. In some embodiments, 5 or fewer modified nucleotides of the second nucleotide sequence are 2′-fluoro nucleotides. In some embodiments, 4 or fewer modified nucleotides of the second nucleotide sequence are 2′-fluoro nucleotides. In some embodiments, 3 or fewer modified nucleotides of the second nucleotide sequence are 2′-fluoro nucleotides. In some embodiments, 2 or fewer modified nucleotides of the second nucleotide sequence are 2′-fluoro nucleotides. In some embodiments, at least one modified nucleotide of the second nucleotide sequence is a 2′-fluoro pyrimidine. In some embodiments, 1, 2, 3, 4, 5, or 6 modified nucleotides of the second nucleotide sequence are 2′-fluoro pyrimidines. In some embodiments, at least one modified nucleotide of the second nucleotide sequence is a 2′-fluoro purine. In some embodiments, 1, 2, 3, 4, 5, or 6 modified nucleotides of the second nucleotide sequence are 2′-fluoro purines. In some embodiments, the 2′-fluoro nucleotide is a 2′-fluoro nucleotide mimic.

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

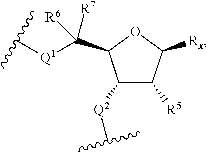

wherein Rₓ is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analog or derivative thereof.

In some embodiments, the 2′-fluoro or 2′-O-methyl nucleotide mimic is a nucleotide mimic of Formula (16)-Formula (20):

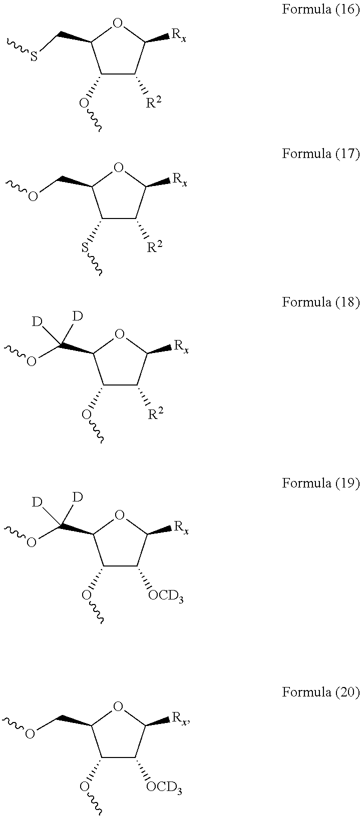

wherein Rₓ is a nucleobase, aryl, heteroaryl, or H and R² is independently F or —OCH, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

In some embodiments, the sense strand, the antisense strand, or both may each independently comprise at least 1, at least 2, at least 3, at least 4, or at least 5 or more modified nucleotide(s) having the following chemical structure:

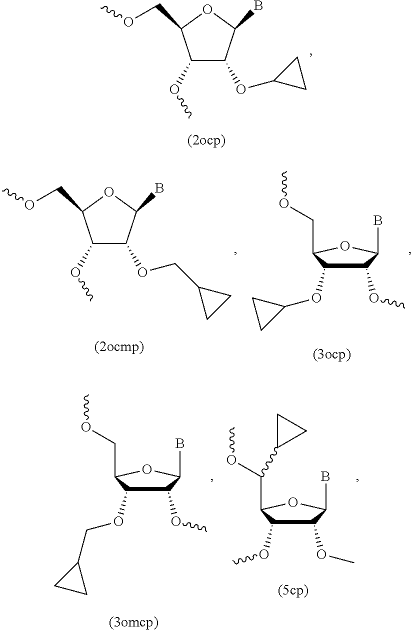

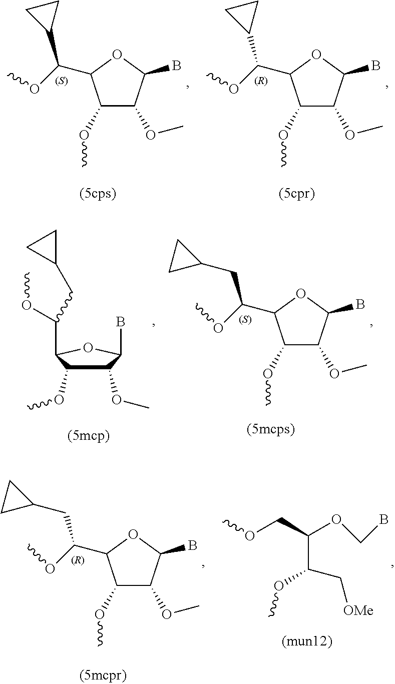

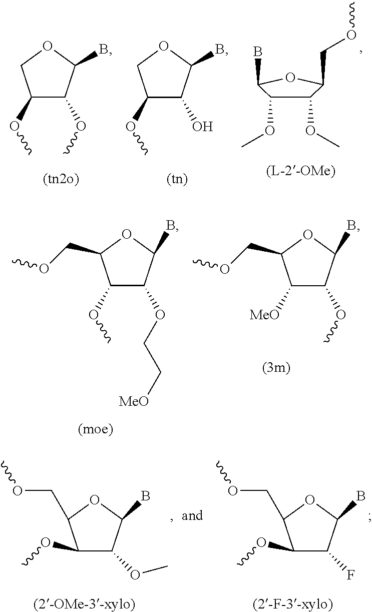

wherein B is a nucleobase, aryl, heteroaryl, or H, and wherein [symbol] represents a phosphodiester linkage, a phosphorothiate linkage, a mesyl phosphoramidate linkage, or H. In some embodiments, the nucleobase is selected from thymine, cytosine, guanine, adenine, uracil, and an analog or derivative thereof.

In some embodiments, the antisense strand, sense strand, or both may each additionally independently comprise at least 1, at least 2, at least 3, at least 4, or at least 5 or more modified nucleotide(s) having the following chemical structure:

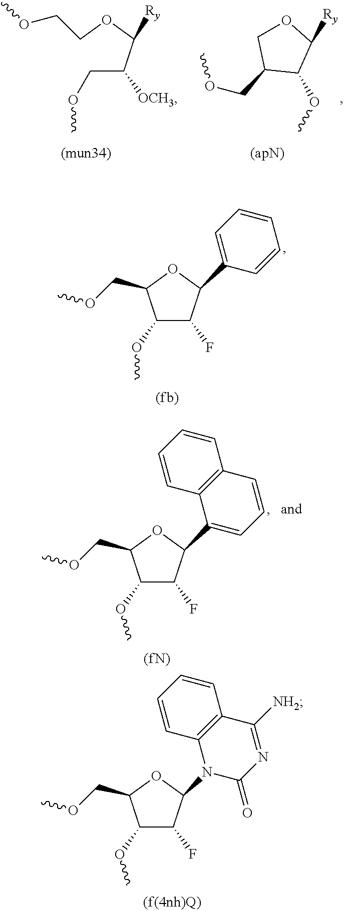

wherein R_{y} is a nucleobase, and wherein [symbol] represents a phosphodiester linkage, a phosphorothioate linkage, a mesyl phosphoroamidate linkage, or H. In some embodiments, the nucleobase is selected from thymine, cytosine, guanine, adenine, uracil, and an analog or derivative thereof.

For the purposes of the present disclosure, the modified nucleotide may be in any position of the antisense strand. In some embodiments, the modified nucleotide may be at position 1, 2, 3, 4, 5, 6, 7, 8, 9, 10, 11, 12, 13, 14, 15, 16, 17, 18, 19, 20, 21, 22, or 23 of the antisense strand relative to the 5′ end.

In some embodiments, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide. In some embodiments, the nucleotide at position 2, 5, 6, 8, 10, 14, 16, 17, and/or 18 from the 5′ end of the second nucleotide sequence is a 2′-fluoro nucleotide. In some embodiments, at least two nucleotides at positions 2, 5, 6, 8, 10, 14, 16, 17, and/or 18 from the 5′ end of the second nucleotide sequence are 2′-fluoro nucleotides. In some embodiments, at least three nucleotides at positions 2, 5, 6, 8, 10, 14, 16, 17, and/or 18 from the 5′ end of the second nucleotide sequence are 2′-fluoro nucleotides. In some embodiments, at least four nucleotides at positions 2, 5, 6, 8, 10, 14, 16, 17, and/or 18 from the 5′ end of the second nucleotide sequence are 2′-fluoro nucleotides. In some embodiments, at least five nucleotides at positions 2, 5, 6, 8, 10, 14, 16, 17, and/or 18 from the 5′ end of the second nucleotide sequence are 2′-fluoro nucleotides. In some embodiments, the nucleotides at positions 2 and/or 14 from the 5′ end of the second nucleotide sequence are 2′-fluoro nucleotides. In some embodiments, the nucleotides at positions 2, 6, and/or 16 from the 5′ end of the second nucleotide sequence are 2′-fluoro nucleotides. In some embodiments, the nucleotides at positions 2, 6, 14, and/or 16 from the 5′ end of the second nucleotide sequence are 2′-fluoro nucleotides. In some embodiments, the nucleotides at positions 2, 6, 10, 14, and/or 18 from the 5′ end of the second nucleotide sequence are 2′-fluoro nucleotides. In some embodiments, the nucleotides at positions 2, 5, 8, 14, and/or 17 from the 5′ end of the second nucleotide sequence are 2′-fluoro nucleotides. In some embodiments, the nucleotide at position 2 from the 5′ end of the second nucleotide sequence is a 2′-fluoro nucleotide. In some embodiments, the nucleotide at position 5 from the 5′ end of the second nucleotide sequence is a 2′-fluoro nucleotide. In some embodiments, the nucleotide at position 6 from the 5′ end of the second nucleotide sequence is a 2′-fluoro nucleotide. In some embodiments, the nucleotide at position 8 from the 5′ end of the second nucleotide sequence is a 2′-fluoro nucleotide. In some embodiments, the nucleotide at position 10 from the 5′ end of the second nucleotide sequence is a 2′-fluoro nucleotide. In some embodiments, the nucleotide at position 14 from the 5′ end of the second nucleotide sequence is a 2′-fluoro nucleotide. In some embodiments, the nucleotide at position 16 from the 5′ end of the second nucleotide sequence is a 2′-fluoro nucleotide. In some embodiments, the nucleotide at position 17 from the 5′ end of the second nucleotide sequence is a 2′-fluoro nucleotide. In some embodiments, the nucleotide at position 18 from the 5′ end of the second nucleotide sequence is a 2′-fluoro nucleotide. In some embodiments, the 2′-fluoro nucleotide is a 2′-fluoro nucleotide mimic.

In some embodiments, the nucleotides in the second nucleotide sequence are arranged in an alternating 1:3 modification pattern, wherein 1 nucleotide is a 2′-fluoro nucleotide and 3 nucleotides are 2′-O-methyl nucleotides, and wherein the alternating 1:3 modification pattern occurs at least 2 times. In some embodiments, the alternating 1:3 modification pattern occurs 2-5 times. In some embodiments, at least two of the alternating 1:3 modification pattern occur consecutively. In some embodiments, at least two of the alternating 1:3 modification pattern occurs nonconsecutively. In some embodiments, at least 1, 2, 3, 4, or 5 alternating 1:3 modification pattern begins at nucleotide position 2, 6, 10, 14, and/or 18 from the 5′ end of the antisense strand. In some embodiments, at least one alternating 1:3 modification pattern begins at nucleotide position 2 from the 5′ end of the antisense strand. In some embodiments, wherein at least one alternating 1:3 modification pattern begins at nucleotide position 6 from the 5′ end of the antisense strand. In some embodiments, at least one alternating 1:3 modification pattern begins at nucleotide position 10 from the 5′ end of the antisense strand. In some embodiments, at least one alternating 1:3 modification pattern begins at nucleotide position 14 from the 5′ end of the antisense strand. In some embodiments, at least one alternating 1:3 modification pattern begins at nucleotide position 18 from the 5′ end of the antisense strand. In some embodiments, the 2′-fluoro nucleotide is a 2′-fluoro nucleotide mimic.

In some embodiments, the nucleotides in the second nucleotide sequence are arranged in an alternating 1:2 modification pattern, wherein 1 nucleotide is a 2′-fluoro nucleotide and 2 nucleotides are 2′-O-methyl nucleotides, and wherein the alternating 1:2 modification pattern occurs at least 2 times. In some embodiments, the alternating 1:2 modification pattern occurs 2-5 times. In some embodiments, at least two of the alternating 1:2 modification pattern occurs consecutively. In some embodiments, at least two of the alternating 1:2 modification pattern occurs nonconsecutively. In some embodiments, at least 1, 2, 3, 4, or 5 alternating 1:2 modification pattern begins at nucleotide position 2, 5, 8, 14, and/or 17 from the 5′ end of the antisense strand. In some embodiments, at least one alternating 1:2 modification pattern begins at nucleotide position 2 from the 5′ end of the antisense strand. In some embodiments, at least one alternating 1:2 modification pattern begins at nucleotide position 5 from the 5′ end of the antisense strand. In some embodiments, at least one alternating 1:2 modification pattern begins at nucleotide position 8 from the 5′ end of the antisense strand. In some embodiments, at least one alternating 1:2 modification pattern begins at nucleotide position 14 from the 5′ end of the antisense strand. In some embodiments, at least one alternating 1:2 modification pattern begins at nucleotide position 17 from the 5′ end of the antisense strand. In some embodiments, the 2′-fluoro nucleotide is a 2′-fluoro nucleotide mimic.

In some embodiments, the second nucleotide sequence comprises, consists of, or consists essentially of ribonucleic acids (RNAs). In some embodiments, the second nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the second nucleotide sequence are independently selected from 2′-O-methyl RNA and 2′-fluoro RNA. In some embodiments, the 2′-fluoro nucleotide is a 2′-fluoro nucleotide mimic.

In some embodiments, the sense strand may further comprise one or more internucleoside linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a sulfo internucleoside linkage.

In some embodiments, the antisense strand may further comprise at least 1, 2, 3, 4, 5, 6, 7, 8, 9, 10, 11, 12, 13, 14, or 15 or more phosphorothioate internucleoside linkages. In some embodiments, the antisense strand comprises 20, 19, 18, 17, 16, 15, 14, 13, 12, 11, 10, 9, 8, 7, 6, 5, 4, or 3 or fewer phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 8 phosphorothioate internucleoside linkages. In some embodiments, the antisense strand comprises 3 to 8 phosphorothioate internucleoside linkages. In some embodiments, the antisense strand comprises 4 to 8 phosphorothioate internucleoside linkages. In some embodiments, at least one phosphorothioate internucleoside linkage is between the nucleotides at positions 1 and 2 from the 5′ end of the second nucleotide sequence. In some embodiments, at least one phosphorothioate internucleoside linkage is between the nucleotides at positions 2 and 3 from the 5′ end of the second nucleotide sequence. In some embodiments, at least one phosphorothioate internucleoside linkage is between the nucleotides at positions 1 and 2 from the 3′ end of the second nucleotide sequence. In some embodiments, at least one phosphorothioate internucleoside linkage is between the nucleotides at positions 2 and 3 from the 3′ end of the second nucleotide sequence. In some embodiments, the antisense strand comprises two phosphorothioate internucleoside linkages between the nucleotides at positions 1 to 3 from the 5′ end of the first nucleotide sequence. In some embodiments, the antisense strand comprises two phosphorothioate internucleoside linkages between the nucleotides at positions 1 to 3 from the 3′ end of the first nucleotide sequence. In some embodiments, the antisense strand comprises (a) two phosphorothioate internucleoside linkages between the nucleotides at positions 1 to 3 from the 5′ end of the first nucleotide sequence; and (b) two phosphorothioate internucleoside linkages between the nucleotides at positions 1 to 3 from the 3′ end of the first nucleotide sequence.

In some embodiments, the antisense strand may further comprise at least 1, 2, 3, 4, 5, 6, 7, 8, 9, 10, 11, 12, 13, 14, or 15 or more mesyl phosphoramidate internucleoside linkages. In some embodiments, the antisense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 8 mesyl phosphoramidate internucleoside linkages. In some embodiments, the antisense strand comprises 3 to 8 mesyl phosphoramidate internucleoside linkages. In some embodiments, the antisense strand comprises 4 to 8 mesyl phosphoramidate internucleoside linkages.

In some embodiments, at least one end of the ds-siNA is a blunt end. In some embodiments, at least one end of the ds-siNA comprises an overhang, wherein the overhang comprises at least one nucleotide. In some embodiments, both ends of the ds-siNA comprise an overhang, wherein the overhang comprises at least one nucleotide. In some embodiments, the overhang comprises 1 to 5 nucleotides, 1 to 4 nucleotides, 1 to 3 nucleotides, or 1 to 2 nucleotides. In some embodiments, the overhang consists of 1 to 2 nucleotides.

In some embodiments, the sense strand may comprise any of the modified nucleotides disclosed in the sub-section titled “Modified Nucleotides” below. In some embodiments, the sense strand may comprise a 5′-stabilized end cap, and the 5′-stabilized end cap may be selected from those disclosed in the sub-section titled “S′-Stabilized End Cap” below.

### Modified Nucleotides

The present disclosure provides oligonucleotides that comprise one or more modified nucleotides disclosed herein. The oligonucleotide may be selected from a short interfering nucleic acid (siNA), an antisense oligonucleotide (ASO), a steric blocker, a short hairpin RNA (shRNA), and an mRNA.

The oligonucleotide may be a siNA, which may comprise a sense strand and an antisense strand. In some embodiments, the sense strands disclosed herein comprise one or more modified nucleotides. In some embodiments, any of the first nucleotide sequences disclosed herein comprise one or more modified nucleotides. In some embodiments, the antisense strands disclosed herein comprise one or more modified nucleotides. In some embodiments, any of the second nucleotide sequences disclosed herein comprise one or more modified nucleotides. In some embodiments, the one or more modified nucleotides is adjacent to the first nucleotide sequence. In some embodiments, at least one modified nucleotide is adjacent to the 5′ end of the first nucleotide sequence. In some embodiments, at least one modified nucleotide is adjacent to the 3′ end of the first nucleotide sequence. In some embodiments, at least one modified nucleotide is adjacent to the 5′ end of the first nucleotide sequence and at least one modified nucleotide is adjacent to the 3′ end of the first nucleotide sequence. In some embodiments, the one or more modified nucleotides is adjacent to the second nucleotide sequence. In some embodiments, at least one modified nucleotide is adjacent to the 5′ end of the second nucleotide sequence. In some embodiments, at least one modified nucleotide is adjacent to the 3′ end of the second nucleotide sequence. In some embodiments, at least one modified nucleotide is adjacent to the 5′ end of the second nucleotide sequence and at least one modified nucleotide is adjacent to the 3′ end of the second nucleotide sequence. In some embodiments, a 2′-O-methyl nucleotide in any of sense strands or first nucleotide sequences disclosed herein is replaced with a modified nucleotide. In some embodiments, a 2′-O-methyl nucleotide in any of antisense strands or second nucleotide sequences disclosed herein is replaced with a modified nucleotide.

In some embodiments, any of the siNA molecules, siNAs, sense strands, first nucleotide sequences, antisense strands, and second nucleotide sequences disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more modified nucleotides. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the siNA molecule, siNA, sense strand, first nucleotide sequence, antisense strand, or second nucleotide sequence are modified nucleotides.

In some embodiments, a modified nucleotide is selected from the group consisting of 2′-fluoro nucleotide, 2′-O-methyl nucleotide, 2′-fluoro nucleotide mimic, 2′-O-methyl nucleotide mimic, 2′-ocp nucleotide, 2′-omcp nucleotide, 3′-ocp nucleotide, 2′-OMe-3′-xylo nucleotide, 2′-F-3′-xylo nucleotide, vmX nucleotide, ganciclovir nucleotide, or denavir nucleotide, a locked nucleic acid, an unlocked nucleic acid, a nucleotide analog, and a nucleotide comprising a modified nucleobase. In some embodiments, the unlocked nucleic acid is a 2′,3′-unlocked nucleic acid. In some embodiments, the unlocked nucleic acid is a 3′,4′-unlocked nucleic acid (e.g., 3′,4′-seco and mun34) in which the furanose ring lacks a bond between the 3′ and 4; carbons.

In some aspects, the siNA of the present disclosure will comprise at least one modified nucleotide selected from:

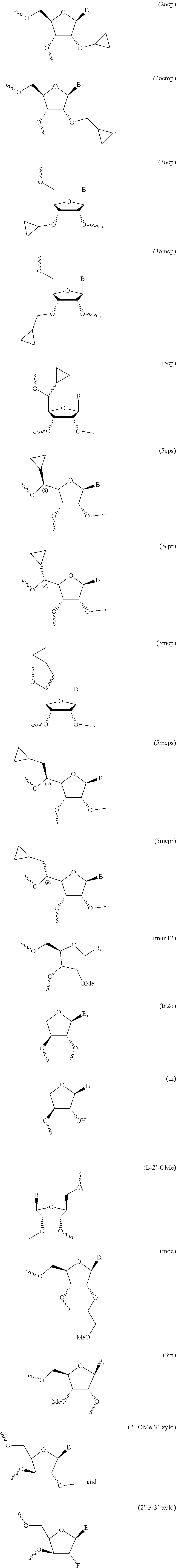

or combinations thereof. In some embodiments, the siNA may comprise at least 2, at least 3, at least 4, or at least 5 or more of these modified nucleotides. In some embodiments, the sense strand may comprise at least 1, at least 2, at least 3, at least 4, or at least 5 or more of:

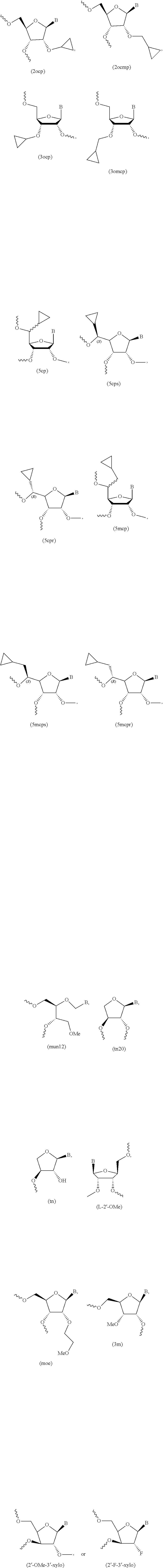

or combinations thereof. In some embodiments, the antisense strand may comprise at least 1, at least 2, at least 3, at least 4, or at least 5 or more

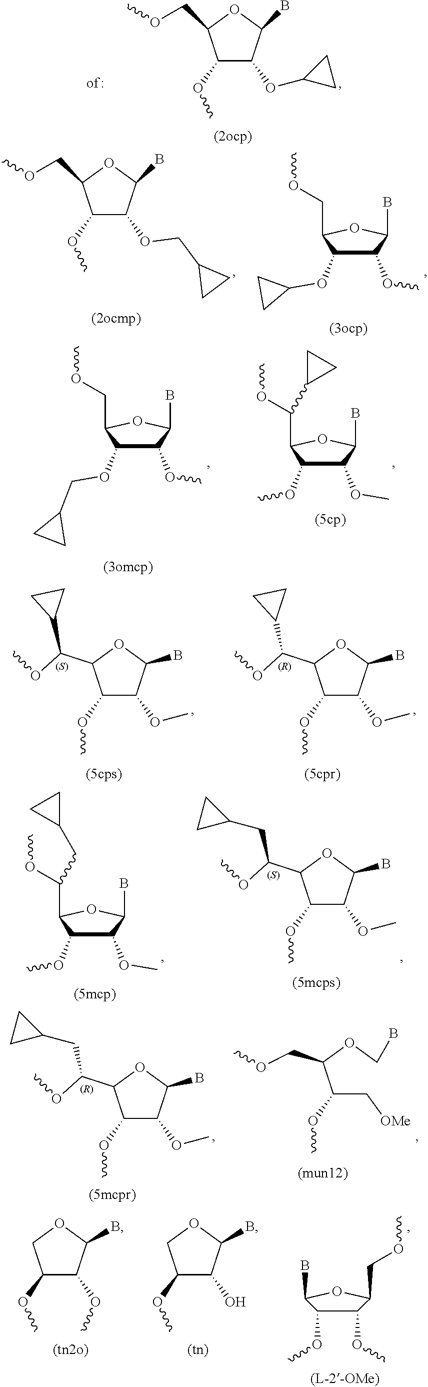

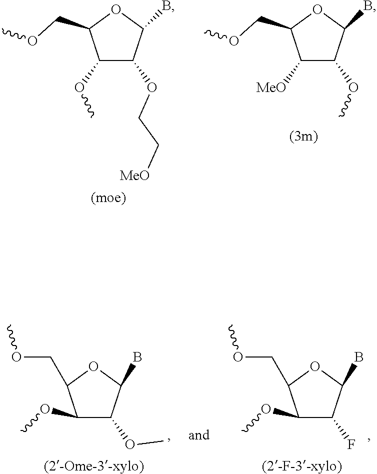

or combinations thereof; wherein B is a nucleobase, aryl, heteroaryl, or H. In some embodiments, the nucleobase is selected from thymine, cytosine, guanine, adenine, uracil, and an analog or derivative thereof.

In some aspects, the siNA of the present disclosure will optionally additionally comprise one or more modified nucleotides selected from:

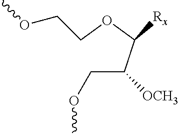

(wherein Rx is a nucleobase, aryl, heteroaryl, or H),

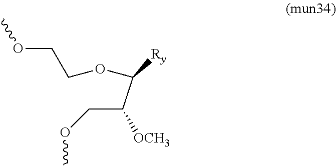

wherein R_{y} is a nucleobase, and

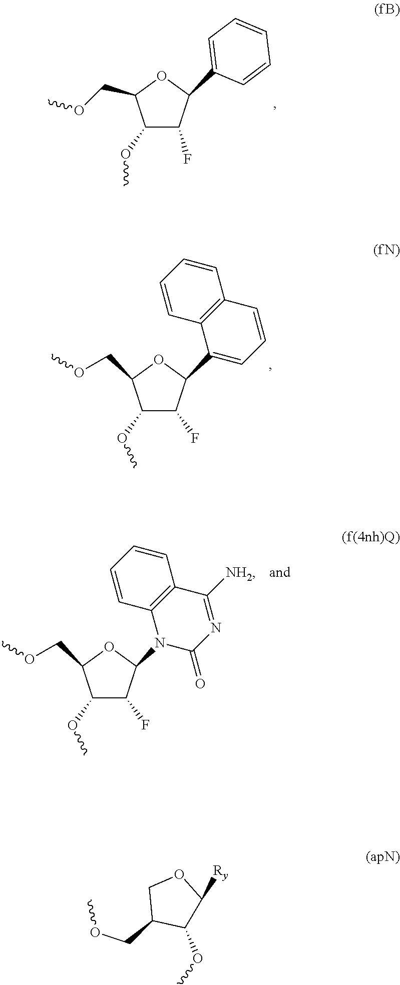

wherein R_{y} is a nucleobase, or combinations thereof. In some embodiments, the siNA may comprise 2, 3, 4, or 5 or more of these modified nucleotides. In some embodiments, the sense strand may optionally additionally comprise one or more modified nucleotides comprising 1, 2, 3, 4, or 5 or more of

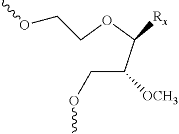

(wherein Rx is a nucleobase, aryl, heteroaryl, or H),

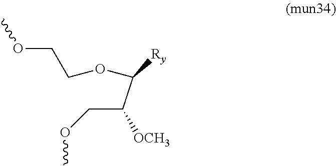

wherein R_{y} is a nucleobase, and

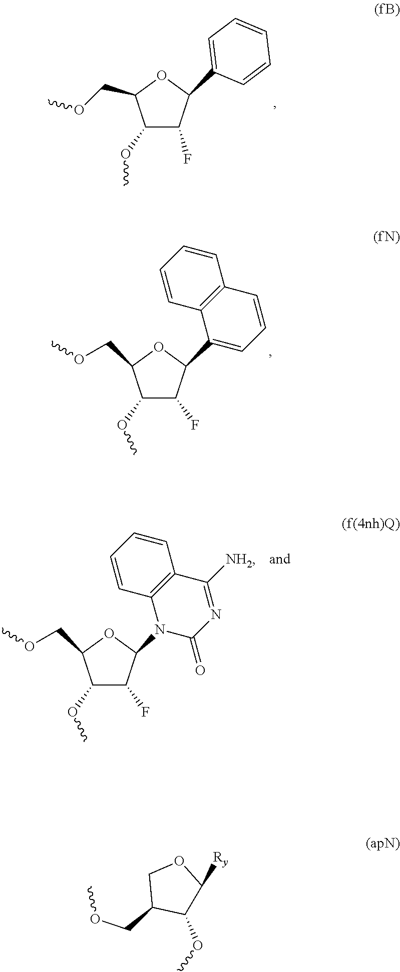

wherein R_{y} is a nucleobase, or combinations thereof. In some embodiments, the antisense strand may comprise 1, 2, 3, 4, or 5 or more of

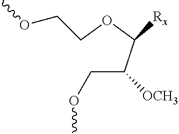

(wherein Rₓ is a nucleobase, aryl, heteroaryl, or H),

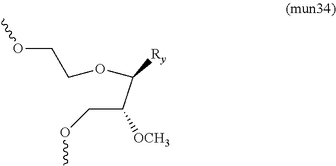

wherein R_{y} is a nucleobase, and

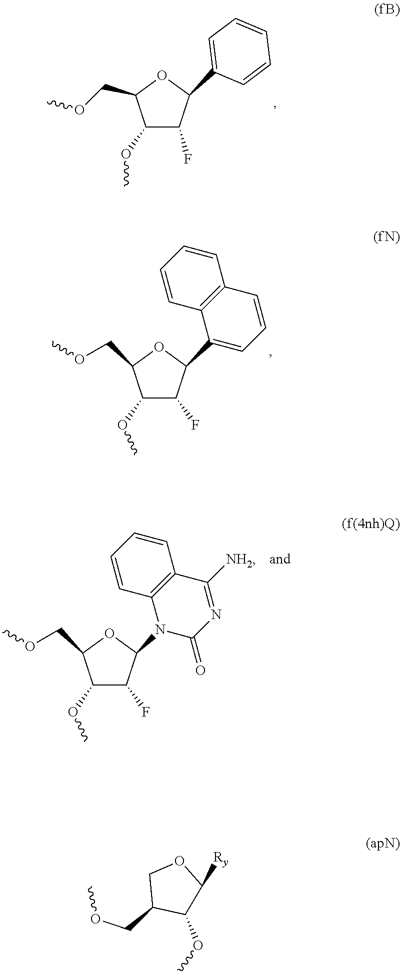

wherein R_{y} is a nucleobase, or combinations thereof. In some embodiments, both the sense strand and the antisense strand may each independently comprise 1, 2, 3, 4, 5 or more of

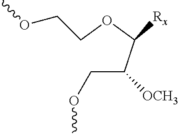

(wherein Rₓ is a nucleobase, aryl, heteroaryl, or H),

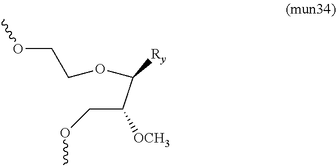

wherein R_{y} is a nucleobase, and

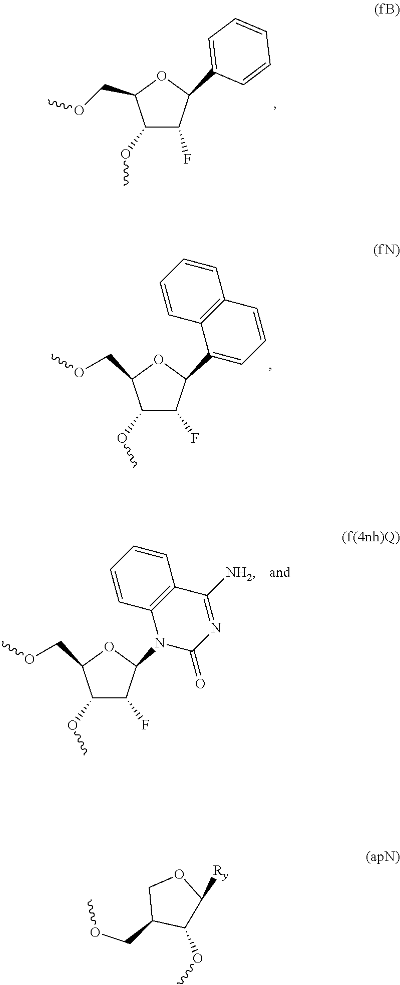

wherein R_{y} is a nucleobase, or combinations thereof. In some embodiments, the nucleobase is selected from thymine, cytosine, guanine, adenine, uracil, and an analog or derivative thereof. For example, in some embodiments of

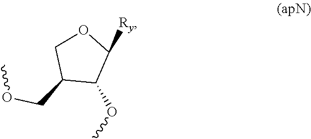

the modified nucleotide may have a structure of

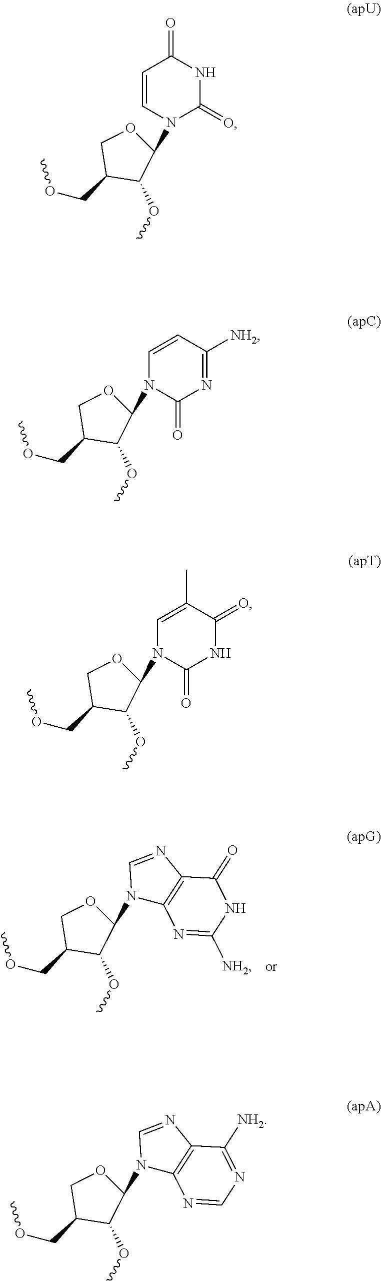

In some embodiments, any of the siNAs disclosed herein may additionally comprise other modified nucleotides, such as 2′-fluoro or 2′-O-methyl nucleotide mimics. For example, the disclosed siNA may comprise at least 1, 2, 3, 4, 5, 6, 7, 8, 9, or 10 or more 2′-fluoro or 2′-O-methyl nucleotide mimics. In some embodiments, any of the sense strands disclosed herein comprise at least 1, 2, 3, 4, 5, 6, 7, 8, 9, or 10 or more 2′-fluoro or 2′-O-methyl nucleotide mimics. In some embodiments, any of the first nucleotide sequences disclosed herein comprise at least 1, 2, 3, 4, 5, 6, 7, 8, 9, or 10 or more 2′-fluoro or 2′-O-methyl nucleotide mimics. In some embodiments, any of the antisense strand disclosed herein comprise at least 1, 2, 3, 4, 5, 6, 7, 8, 9, or 10 or more 2′-fluoro or 2′-O-methyl nucleotide mimics. In some embodiments, any of the second nucleotide sequences disclosed herein comprise at least 1, 2, 3, 4, 5, 6, 7, 8, 9, or 10 or more 2′-fluoro or 2′-O-methyl nucleotide mimics. In some embodiments, the 2′-fluoro or 2′-O-methyl nucleotide mimic is a nucleotide mimic of Formula (16)-Formula (20):

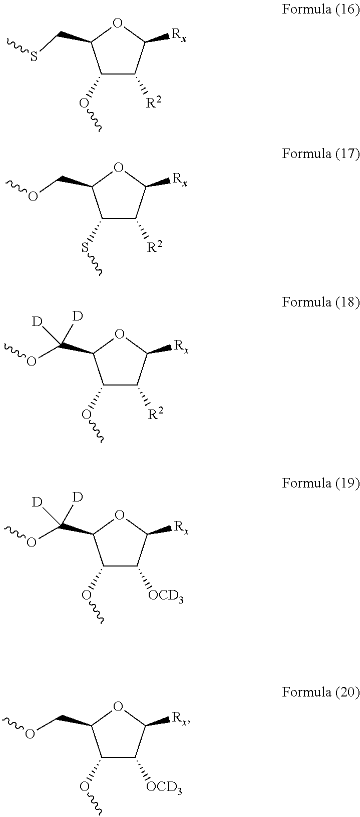

wherein Rₓ is a nucleobase, aryl, heteroaryl, or H and R² is independently F or —OCH₃. In some embodiments, the nucleobase is selected from thymine, cytosine, guanine, adenine, uracil, and an analog or derivative thereof.

In some embodiments, the siNA molecules disclosed herein comprise at least one 2′-fluoro nucleotide, at least one 2′-O-methyl nucleotide, and at least one 2′-fluoro or 2′-O-methyl nucleotide mimic. In some embodiments, the at least one 2′-fluoro or 2′-O-methyl nucleotide mimic is adjacent to the first nucleotide sequence. In some embodiments, the at least one 2′-fluoro or 2′-O-methyl nucleotide mimic is adjacent to the 5′ end of first nucleotide sequence. In some embodiments, the at least one 2′-fluoro or 2′-O-methyl nucleotide mimic is adjacent to the 3′ end of first nucleotide sequence. In some embodiments, the at least one 2′-fluoro or 2′-O-methyl nucleotide mimic is adjacent to the second nucleotide sequence. In some embodiments, the at least one 2′-fluoro or 2′-O-methyl nucleotide mimic is adjacent to the 5′ end of second nucleotide sequence. In some embodiments, the at least one 2′-fluoro or 2′-O-methyl nucleotide mimic is adjacent to the 3′ end of second nucleotide sequence. In some embodiments, the first nucleotide sequence does not comprise a 2′-fluoro nucleotide mimic. In some embodiments, the first nucleotide sequence does not comprise a 2′-O-methyl nucleotide mimic. In some embodiments, the second nucleotide sequence does not comprise a 2′-fluoro nucleotide mimic. In some embodiments, the second nucleotide sequence does not comprise a 2′-O-methyl nucleotide mimic.

In some embodiments, any of the siNAs, sense strands, first nucleotide sequences, antisense strands, or second nucleotide sequences disclosed herein may optionally comprise at least one modified nucleotide that is

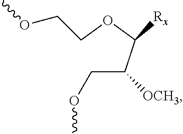

wherein Rx is a nucleobase, aryl, heteroaryl, or H; or

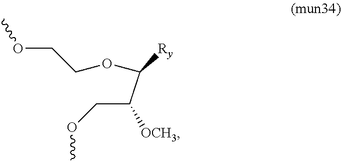

wherein R_{y} is a nucleobase.

In some embodiments, any of the siNAs, sense strands, first nucleotide sequences, antisense strands, or second nucleotide sequences disclosed herein may optionally comprise at least one modified nucleotide that is

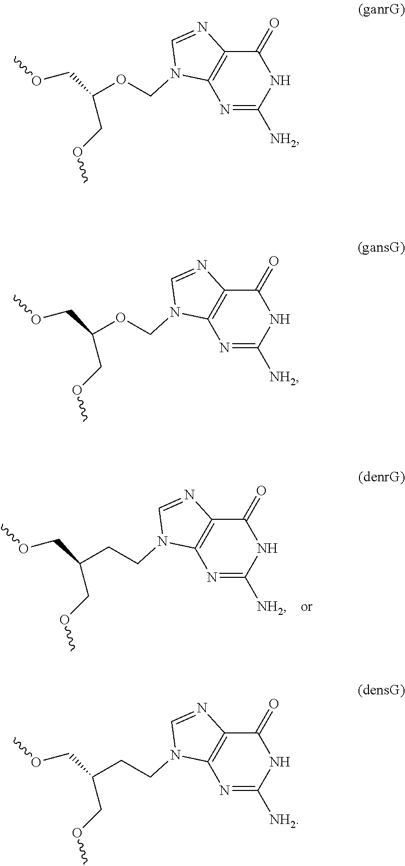

wherein B is a nucleobase, aryl, heteroaryl, or H; wherein [symbol] represents a phosphodiester linkage, a phosphorothicate linkage, or a mesyl phosphoroamidate linkage.

### Phosphorylation Blocker

Further disclosed herein are siNA molecules comprising a phosphorylation blocker. In some embodiments, a 2′-O-methyl nucleotide in any of sense strands or first nucleotide sequences disclosed herein is replaced with a nucleotide containing a phosphorylation blocker. In some embodiments, a 2′-O-methyl, 2′-ocp, or 2′-omcp nucleotide in any of antisense strands or second nucleotide sequences disclosed herein is replaced with a nucleotide containing a phosphorylation blocker. In some embodiments, a 2′-O-methyl, 2′-ocp, or 2′-omcp nucleotide in any of sense strands or first nucleotide sequences disclosed herein is further modified to contain a phosphorylation blocker. In some embodiments, a 2′-O-methyl, 2′-ocp, or 2′-omcp nucleotide in any of antisense strands or second nucleotide sequences disclosed herein is further modified to contain a phosphorylation blocker.

In some embodiments, any of the siNA molecules disclosed herein comprise a phosphorylation blocker of Formula (IV):

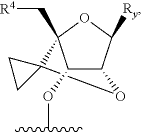

wherein R_{y} is a nucleobase, R⁴ is —O—R³⁰ of —NR³¹R³², R³⁰ is C₁-C₈ substituted or unsubstituted alkyl; and R³¹ and R³² together with the nitrogen to which they are attached form a substituted or unsubstituted heterocyclic ring. In some embodiments, the nucleobase is selected from thymine, cytosine, guanine, adenine, uracil, and an analog or derivative thereof.

In some embodiments, any of the siNA molecules disclosed herein comprise a phosphorylation blocker of Formula (IV):

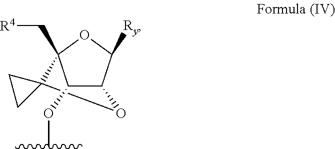

wherein R_{y} is a nucleobase, and R⁴ is —OCH₃ or —N(CH₂CH₂)₂O. In some embodiments, the nucleobase is selected from thymine, cytosine, guanine, adenine, uracil, and an analog or derivative thereof.

In some embodiments, a siNA molecule comprises (a) a phosphorylation blocker of Formula (IV):

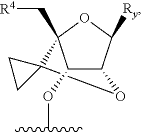

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring; and (b) a short interfering nucleic acid (siNA), wherein the phosphorylation blocker is conjugated to the siNA. In some embodiments, the nucleobase is selected from thymine, cytosine, guanine, adenine, uracil, and an analog or derivative thereof.

In some embodiments, a siNA molecule comprises (a) a phosphorylation blocker of Formula (IV):

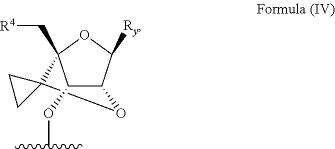

wherein R_{y} is a nucleobase, and R⁴ is —OCH₃ or —N(CH₂CH₂)₂O; and (b) a short interfering nucleic acid (siNA), wherein the phosphorylation blocker is conjugated to the siNA.

In some embodiments, the phosphorylation blocker is attached to the 3′ end of the sense strand or first nucleotide sequence. In some embodiments, the phosphorylation blocker is attached to the 3′ end of the sense strand or first nucleotide sequence via 1, 2, 3, 4, or 5 or more linkers. In some embodiments, the phosphorylation blocker is attached to the 5′ end of the sense strand or first nucleotide sequence. In some embodiments, the phosphorylation blocker is attached to the 5′ end of the sense strand or first nucleotide sequence via 1, 2, 3, 4, or 5 or more linkers. In some embodiments, the phosphorylation blocker is attached to the 3′ end of the antisense strand or second nucleotide sequence. In some embodiments, the phosphorylation blocker is attached to the 3′ end of the antisense strand or second nucleotide sequence via 1, 2, 3, 4, or 5 or more linkers. In some embodiments, the phosphorylation blocker is attached to the 5′ end of the antisense strand or second nucleotide sequence. In some embodiments, the phosphorylation blocker is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, mesyl phosphoramidate linker and phosphorodithioate linker.

### Conjugated Moiety

Further disclosed herein are siNA molecules comprising a conjugated moiety. In some embodiments, the conjugated moiety is selected from galactosamine, peptides, proteins, sterols, lipids, phospholipids, biotin, phenoxazines, active drug substance, cholesterols, phenanthridine, anthraquinone, acridine, fluoresces, rhodamines, coumarins, and dyes. In some embodiments, the conjugated moiety is attached to the 3′ end of the sense strand or first nucleotide sequence. In some embodiments, the conjugated moiety is attached to the 3′ end of the sense strand or first nucleotide sequence via 1, 2, 3, 4, or 5 or more linkers. In some embodiments, the conjugated moiety is attached to the 5′ end of the sense strand or first nucleotide sequence. In some embodiments, the conjugated moiety is attached to the S′ end of the sense strand or first nucleotide sequence via 1, 2, 3, 4, or 5 or more linkers. In some embodiments, the conjugated moiety is attached to the 3′ end of the antisense strand or second nucleotide sequence. In some embodiments, the conjugated moiety is attached to the 3′ end of the antisense strand or second nucleotide sequence via 1, 2, 3, 4, or 5 or more linkers. In some embodiments, the conjugated moiety is attached to the S′ end of the antisense strand or second nucleotide sequence. In some embodiments, the conjugated moiety is attached to the S′ end of the antisense strand or second nucleotide sequence via 1, 2, 3, 4, or 5 or more linkers. In some embodiments, the one or more linkers are independently selected from the group consisting of a phosphodiester linker, phosphorothicate linker, phosphorodithioate linker, and mesyl phosphoramidate linker.

In some embodiments, the conjugated moiety is galactosamine. In some embodiments, any of the siNAs disclosed herein are attached to a conjugated moiety that is galactosamine. In some embodiments, the galactosamine is N-acetylgalactosamine (GalNAc). In some embodiments, any of the siNA molecules disclosed herein comprise GalNAc. In some embodiments, the GalNAc is of Formula (VI):

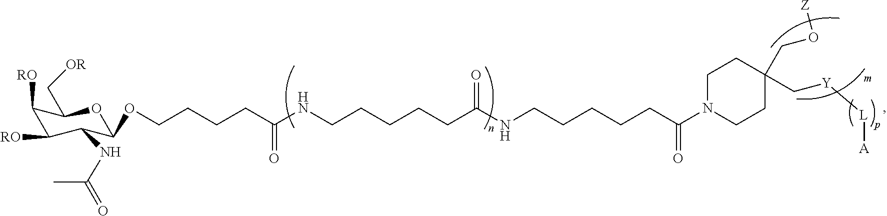

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. In some embodiments, the first protecting group is acetyl. In some embodiments, the second protecting group is trimethoxytrityl (TMT). In some embodiments, the activated group is a phosphoramidite group. In some embodiments, the phosphoramidite group is a cyanoethoxy N,N-diisopropylphosphoramidite group. In some embodiments, the linker is a C₆—NH₂ group. In some embodiments, A is a short interfering nucleic acid (siNA) or siNA molecule. In some embodiments, m is 3. In some embodiments, R is H, Z is H, and n is 1. In some embodiments, R is H, Z is H, and n is 2.

In some embodiments, the GalNAc is Formula (VII):

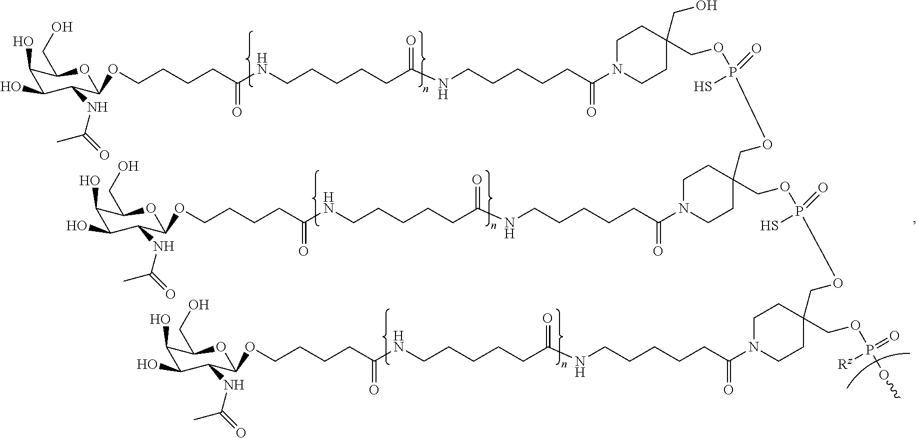

wherein R^{z} is OH or SH; and each n is independently 1 or 2. In some embodiments, the targeting ligand may be a GalNAc targeting ligand may comprise 1, 2, 3, 4, 5 or 6 GalNAc units. In some embodiments, the targeting ligand may be a GalNAc selected from GalNAc2, GalNAc3, GalNAc4 (the GalNAc of Formula VII, wherein n=1 and R^{z}=OH), GalNAc5, and GalNAc6.

In some embodiments, the GalNAc may be GalNAc amidite (i.e., compound 40-9, see Example 22), GalNAc 4 CPG (i.e., compound 40-8, see Example 22 and Example 23), GalNAc phophoramidite, or GalNAc4-ps-GalNAc4-ps-GalNAc4. These GalNAc moieties are shown below:

GalNAc 4 moieties

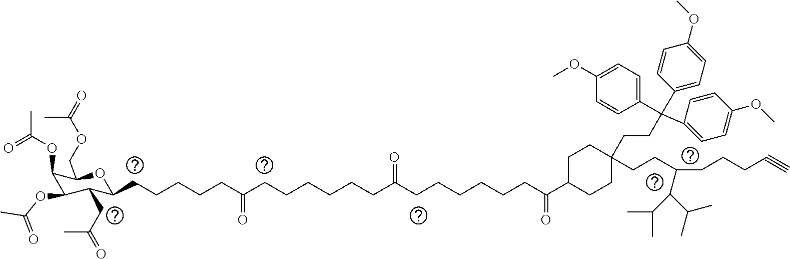

GalNAc4 phosphoramidite

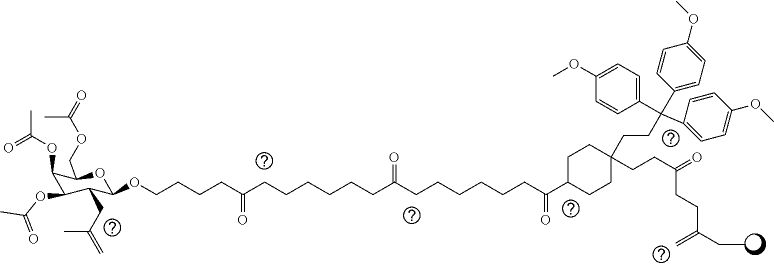

GalNAc4 CPG

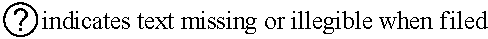

GalNAc3, GalNAc4, GalNAc5 and GalNAc6 may be conjugated to an siNA disclosed herein during synthesis with 1 2, or 3 moieties. Further GalNAc moieties, such as GalNAc1 and GalNAc2, can be used to form 5′ and 3′-GalNAc using post synthesis conjugation.

GalNAc Phosphoramidites
GalNAc building blocks 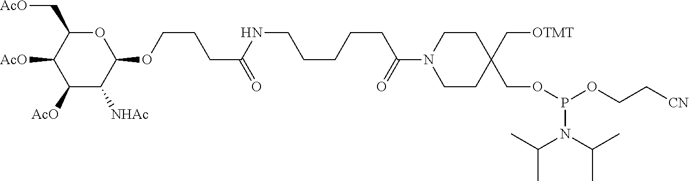
GalNAc-3 phosphoramidite 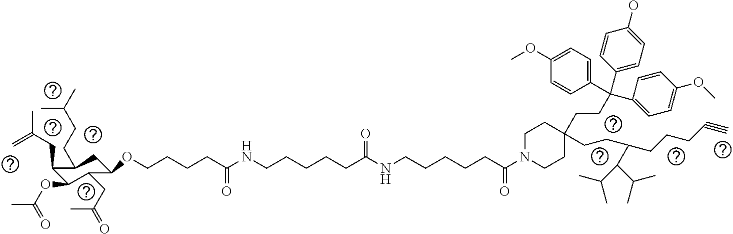
GalNAc-4 phosphoramidite 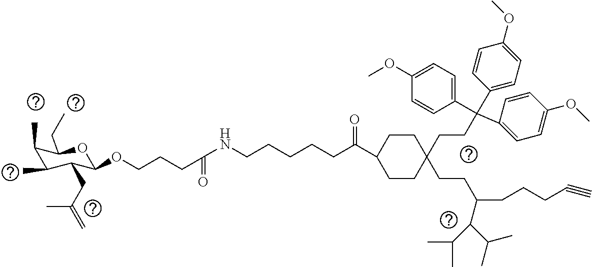
GalNAc-5 phosphoramidite 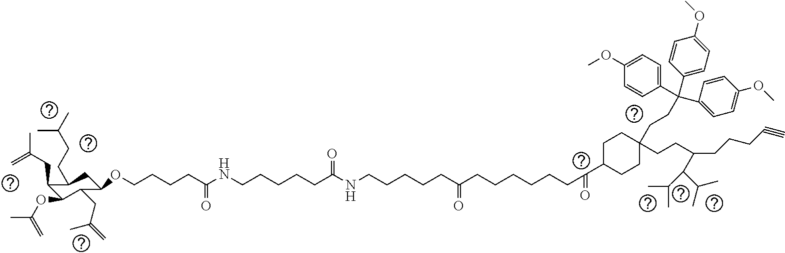
GalNAc-6 phosphoramidite 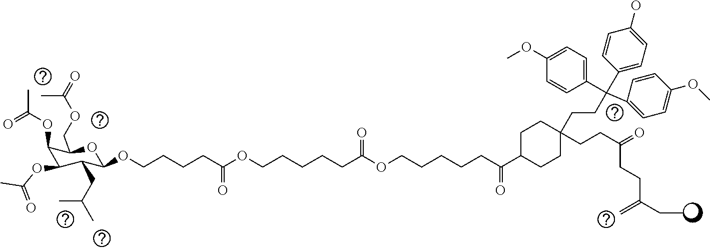
GalNAc4 CPG
After Attachment to Oligos (Nomenclature) 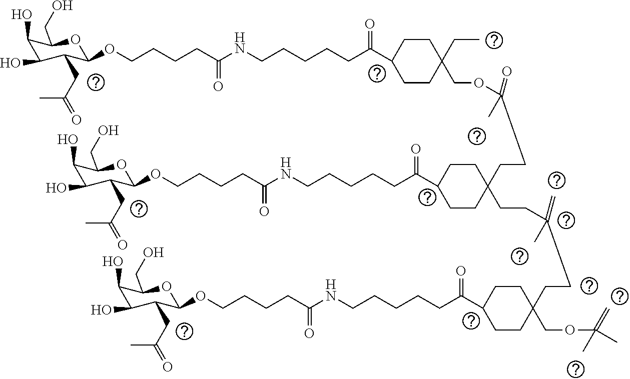
(GalNAc3-(PS)2-p) 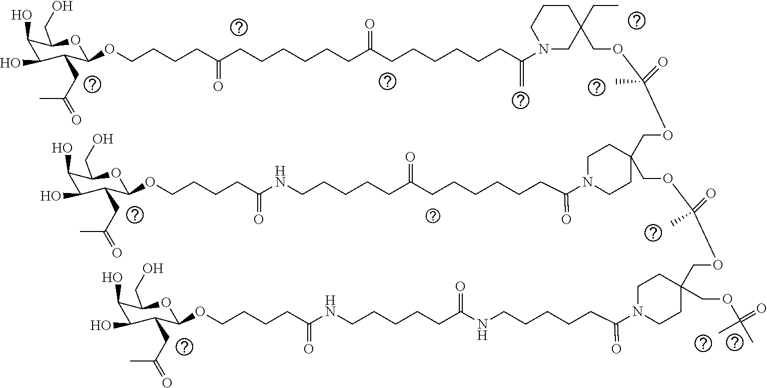
(GalNAc4-(PS)2-p) 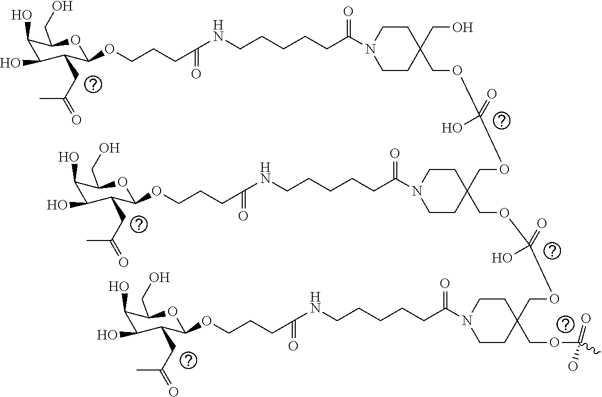
(GalNAc5-(PS)2-p) 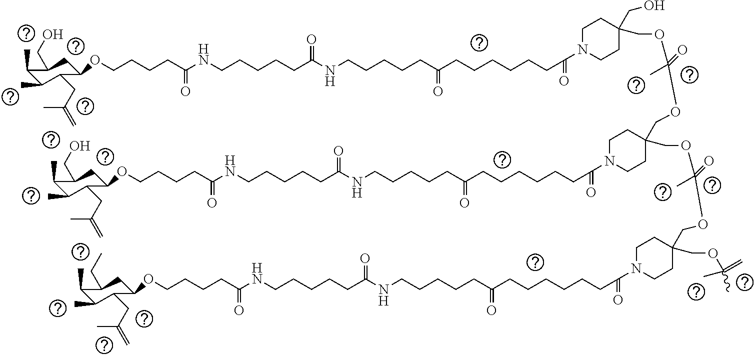
(GalNAc6-(PS)2-p) 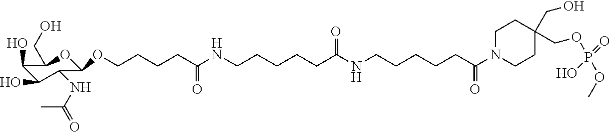
Mono GaINAc4

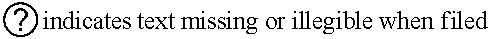

In some embodiments, the galactosamine is attached to the 3′ end of the sense strand or first nucleotide sequence. In some embodiments, the galactosamine is attached to the 3′ end of the sense strand or first nucleotide sequence via 1, 2, 3, 4, or 5 or more linkers. In some embodiments, the galactosamine is attached to the 5′ end of the sense strand or first nucleotide sequence. In some embodiments, the galactosamine is attached to the 5′ end of the sense strand or first nucleotide sequence via 1, 2, 3, 4, or 5 or more linkers. In some embodiments, the galactosamine is attached to the 3′ end of the antisense strand or second nucleotide sequence. In some embodiments, the galactosamine is attached to the 3′ end of the antisense strand or second nucleotide sequence via 1, 2, 3, 4, or 5 or more linkers. In some embodiments, the galactosamine is attached to the 5′ end of the antisense strand or second nucleotide sequence. In some embodiments, the galactosamine is attached to the 5′ end of the antisense strand or second nucleotide sequence via 1, 2, 3, 4, or 5 or more linkers.

In some embodiments, the one or more linkers are independently selected from the group consisting of a phosphodiester (p or po) linker, phosphorothioate (ps) linker, mesyl phosphoramidate linker (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

In some embodiments, the conjugated moiety is a lipid moiety. In some embodiments, any of the siNAs disclosed herein are attached to a conjugated moiety that is a lipid moiety. Examples of lipid moieties include, but are not limited to, a cholesterol moiety, a thioether, e.g., hexyl-S-tritylthiol, a thiocholesterol, an aliphatic chain, e.g., dodecandiol or undecyl residues a phospholipid, e.g., di-hexadecyl-rac-glycerol or triethylammonium 1-di-O-hexadecyl-rac-glycero-S—H-phosphonate, a polyamine or a polyethylene glycol chain, adamantane acetic acid, a palmityl moiety, or an octadecylamine or hexylamino-carbonyl-oxycholesterol moiety.

In some embodiments, the conjugated moiety is an active drug substance. In some embodiments, any of the siNAs disclosed herein are attached to a conjugated moiety that is an active drug substance. Examples of active drug substances include, but are not limited to, aspirin, warfarin, phenylbutazone, ibuprofen, suprofen, fenbufen, ketoprofen, (5)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

### 5′-Stabilized End Cap

Further disclosed herein are oligonucleotides (e.g., siNA) comprising a 5′-stabilized end cap. As used herein the terms “5′-stabilized end cap” and “5′ end cap” are used interchangeably. In some embodiments, a 2′-O-methyl, 2′-ocp, or 2′-omcp nucleotide in any of sense strands or first nucleotide sequences disclosed herein is replaced with a nucleotide containing a 5′-stabilized end cap. In some embodiments, a 2′-O-methyl, 2′-ocp, or 2′-omcp nucleotide in any of antisense strands or second nucleotide sequences disclosed herein is replaced with a nucleotide containing a 5′-stabilized end cap. In some embodiments, a 2′-O-methyl, 2′-ocp, or 2′-omcp nucleotide in any of sense strands or first nucleotide sequences disclosed herein is further modified to contain a 5′-stabilized end cap. In some embodiments, a 2′-O-methyl, 2′-ocp, or 2′-omcp nucleotide in any of antisense strands or second nucleotide sequences disclosed herein is further modified to contain a 5′-stabilized end cap.

In some embodiments, the 5′-stabilized end cap is a 5′ phosphate mimic. In some embodiments, the 5′-stabilized end cap is a modified 5′ phosphate mimic. In some embodiments, the modified 5′ phosphate is a chemically modified 5′ phosphate. In some embodiments, the 5′-stabilized end cap is a 5′-vinyl phosphonate. In some embodiments, the 5′-vinyl phosphonate is a 5′-(E)-vinyl phosphonate or 5′-(Z)-vinyl phosphonate. In some embodiments, the 5′-vinyl phosphonate is a deuterated vinyl phosphonate. In some embodiments, the deuterated vinyl phosphonate is a mono-deuterated vinyl phosphonate. In some embodiments, the deuterated vinyl phosphonate is a di-deuterated vinyl phosphonate. In some embodiments, the 5′-stabilized end cap is a phosphate mimic. Examples of phosphate mimics are disclosed in Parmar et al., J Med Chem, 201861 (3): 734-744, International Publication Nos. WO2018/045317 and WO2018/044350, and U.S. Pat. No. 10,087,210, each of which is incorporated by reference in its entirety.

In some aspects, the present disclosure provides a short interfering nucleic acid (siNA), comprising a sense strand and an antisense strand, wherein the antisense strand comprises a 5′vinyl phosphonate dimer moiety comprising a structure of:

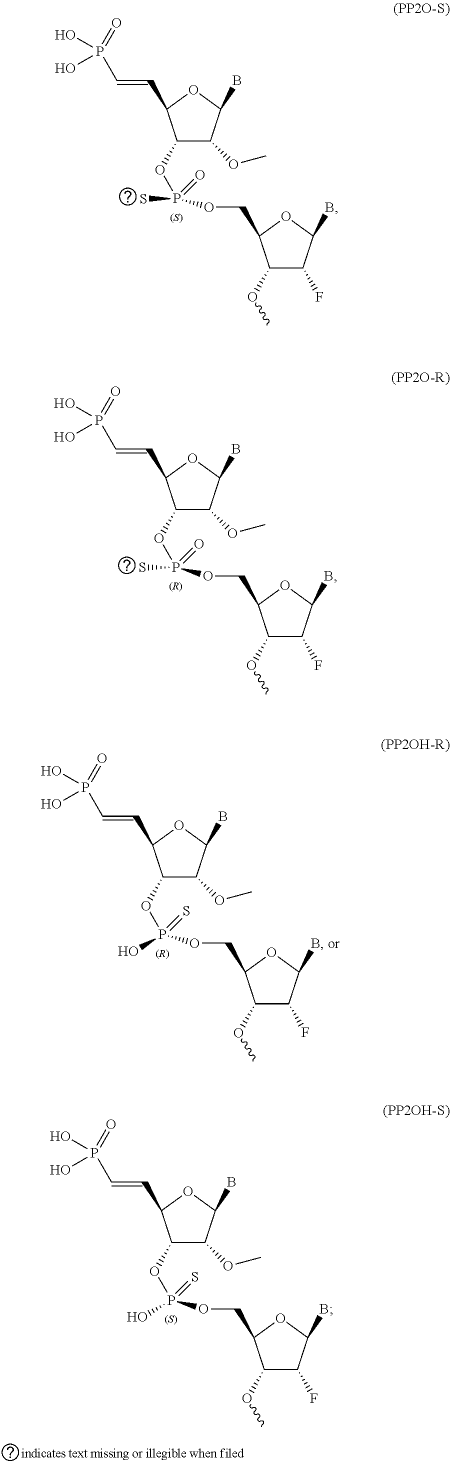

wherein each B is independently selected from a nucleobase, aryl, heteroaryl, and H; wherein [symbol] represents a phosphodiester linkage, a phosphorothioate linkage, or a mesyl phosphoroamidate linkage. In some embodiments, the chiral center is in an S configuration. In some embodiments, the chiral center is in an R configuration. In some embodiments, each B of the vinyl phosphonate dimer moiety may be the same nucleobase, whereas in some embodiments, each B may be a different nucleobase. The nucleobase may be selected from thymine, cytosine, guanine, adenine, uracil, and an analog or derivative thereof. In some embodiments, each B is independently thymine, cytosine, guanine, adenine, or uracil. For example, a vinyl phosphonate dimer of the present disclosure may comprise two different nucleobases, as shown in the following structures:

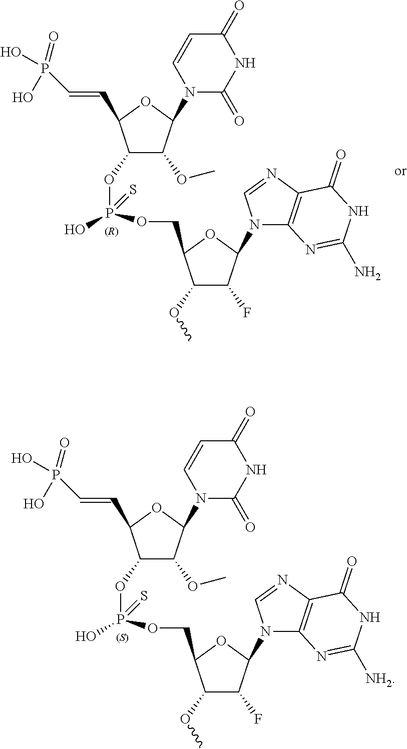

In some aspects, the present disclosure provides siNA optionally comprising a nucleotide phosphate mimic selected from:

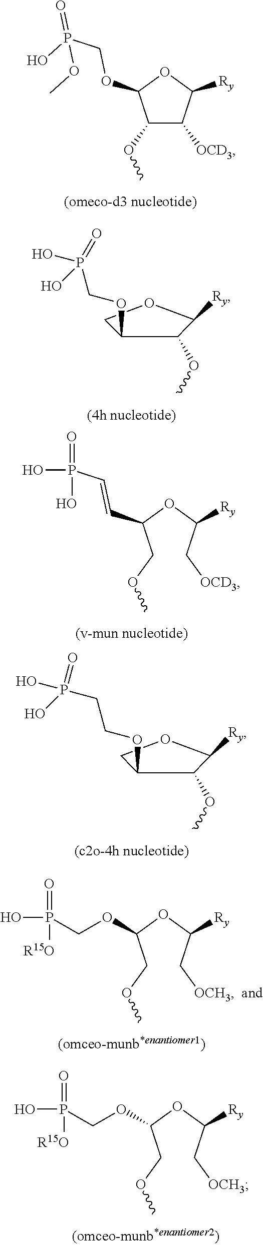

wherein R_{y} is a nucleobase and R¹⁵ is H or CH₃. In some embodiments, the nucleobase is selected from thymine, cytosine, guanine, adenine, uracil, and an analog or derivative thereof. In some embodiments, the disclosed nucleotide phosphate mimics include, but are not limited to, the structures:

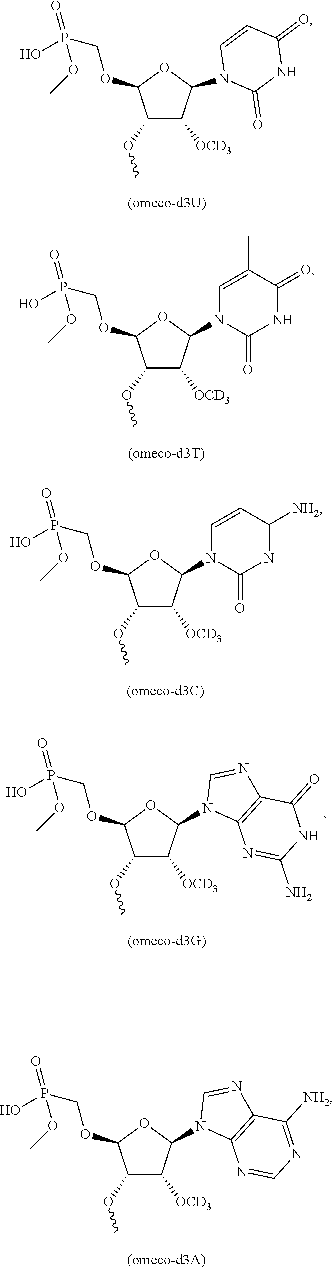

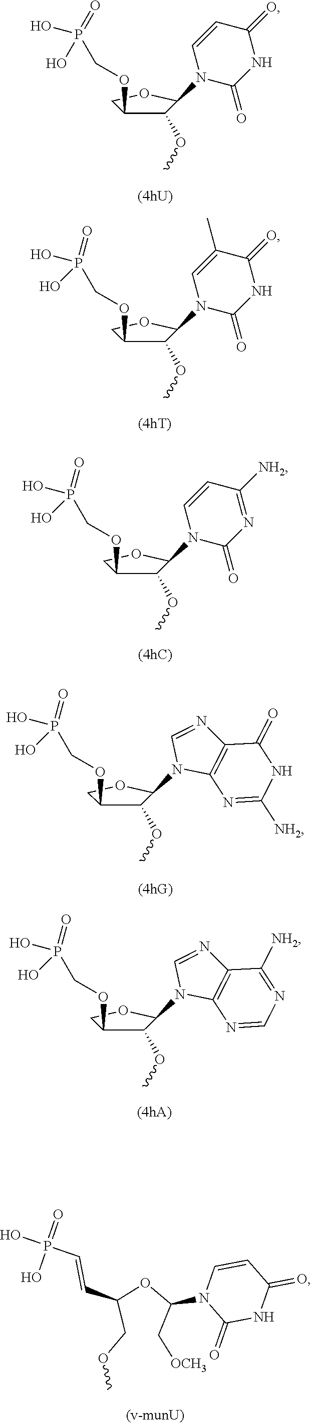

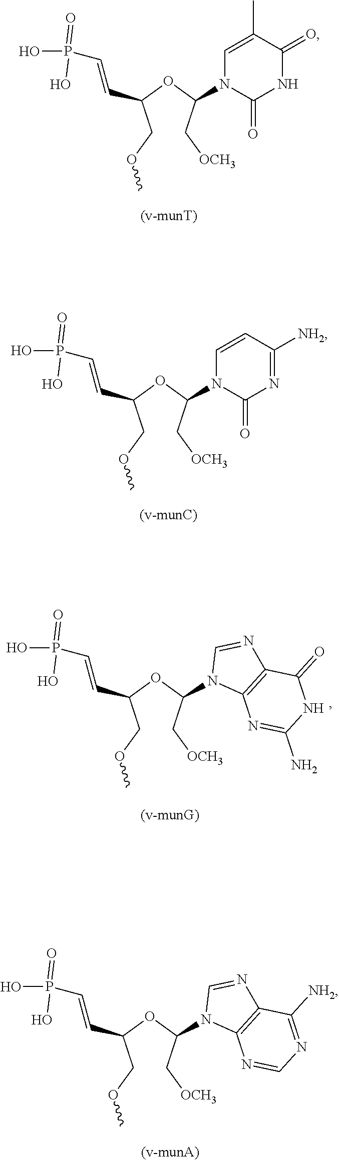

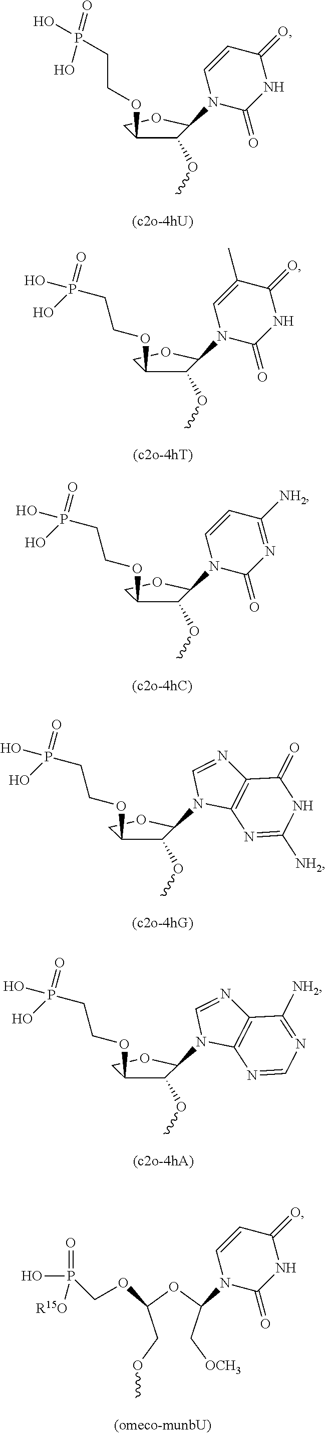

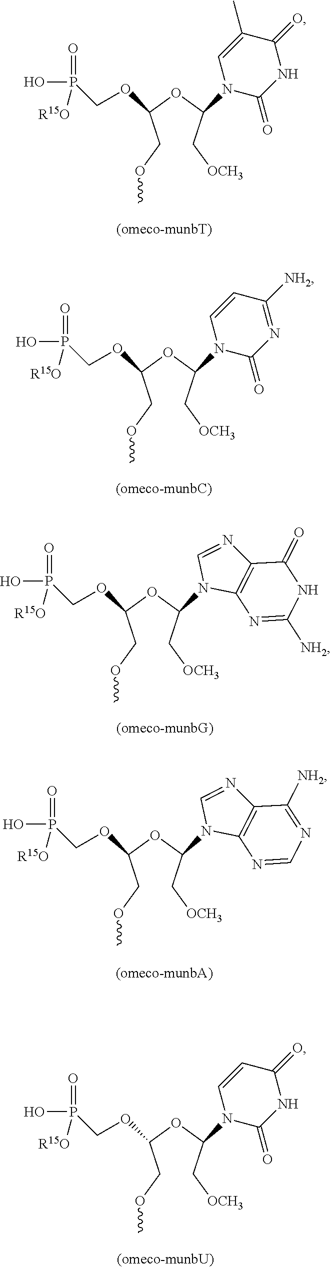

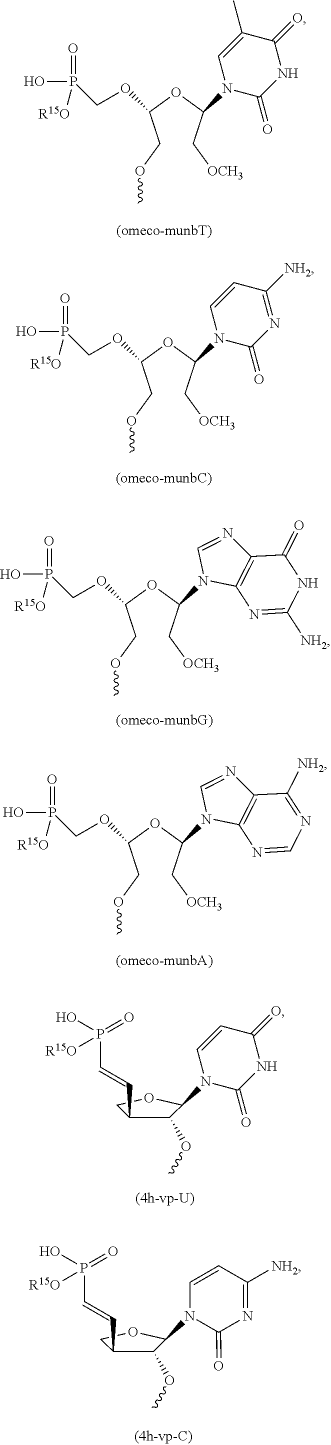

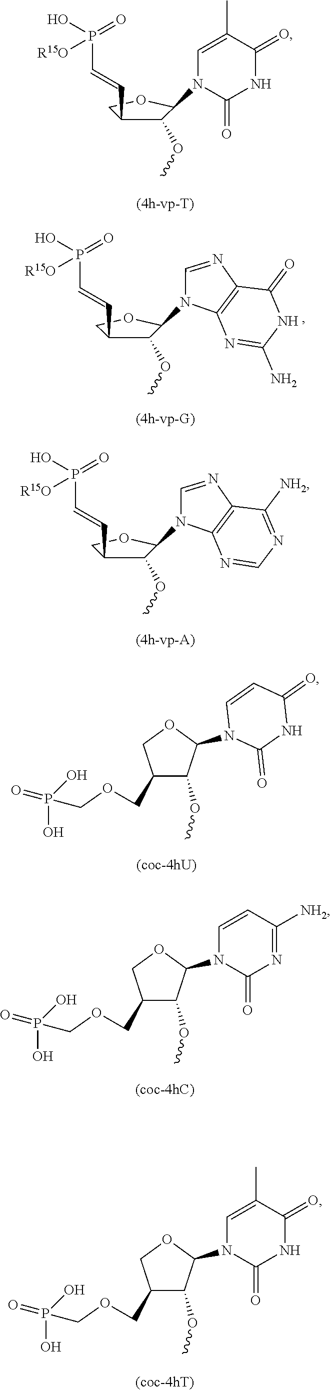

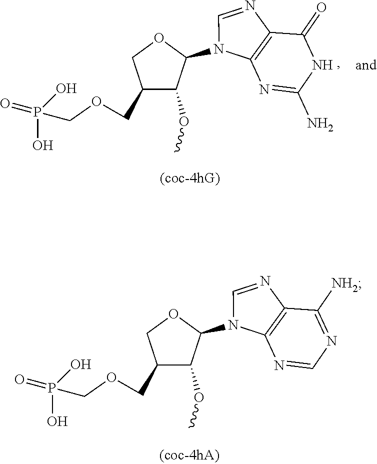

wherein R¹⁵ is H or CH₃.

In some aspects, the present disclosure provides siNA optionally comprising a nucleotide phosphate mimic selected from:

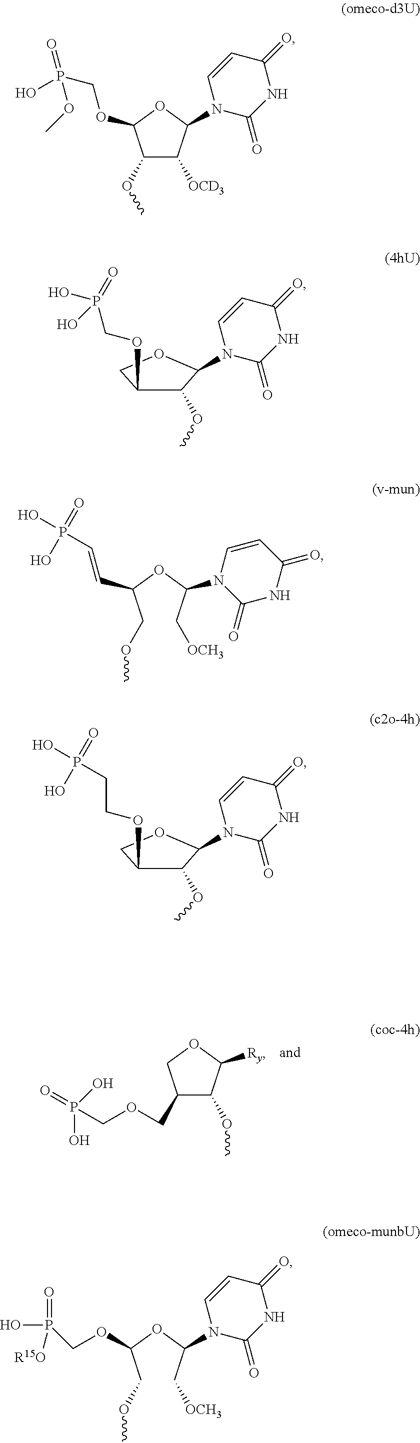

when R¹⁵ is CH₃); where R¹⁵ is H or CH₃. In some embodiments, one of these novel nucleotide phosphate mimics (e.g., omeco-d3 nucleotide, 4h nucleotide, v-mun nucleotide, c20-4 h nucleotide, coc-4b nucleotide, omeco-munb nucleotide, or d2vm nucleotide) may be located at the S′ end of the antisense strand; however, these novel nucleotide phosphate mimics may also be incorporated at the S′ end of the sense strand, the 3′ end of the antisense strand, or the 3′ end of the sense strand.

Additionally or alternatively, the siNA molecules disclosed herein may comprise in the sense strand, the antisense strand, or both a S′-stabilized end cap of Formula (Ia):

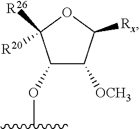

wherein Rₓ is H, a nucleobase, aryl, or heteroaryl; R²⁶ is

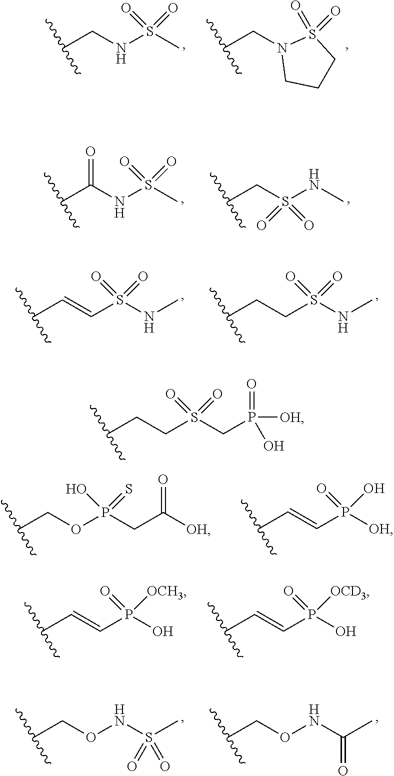

—CH═CD-Z, —CD═CH—Z, —CD═CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH), —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group, R²³ is hydrogen or C₁-C₆ alkyl; R²⁴ is —SO₂R²⁵ or —C(O) R²⁵; or R²³ and R²⁴ together with the nitrogen to which they are attached form a substituted or unsubstituted heterocyclic ring; R₂₅ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a S′-stabilized end cap of Formula (Ib):

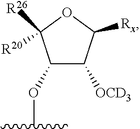

wherein Rₓ is H, a nucleobase, aryl, or heteroaryl; R²⁶ is

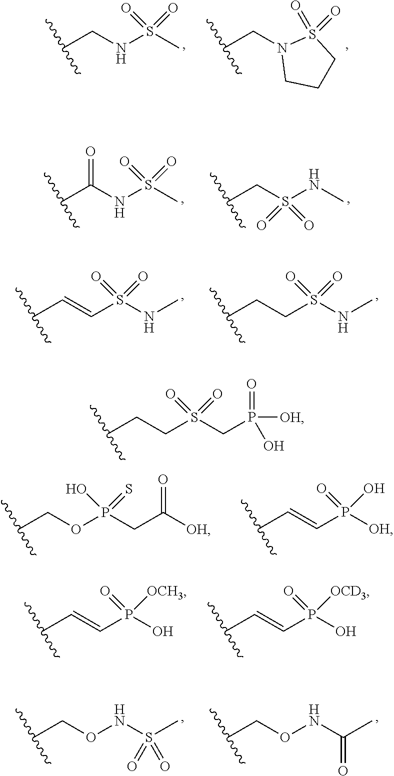

—CH═CD—Z, —CD═CH—Z, —CD═CD—Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₅ alkenylene)-7; n is 1, 2, 3, or 4; Z is —ONR²³R²⁴, —OP(O)OH(CH₃)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, —NR²¹SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O) R²⁵, or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R′ is an aryl. In some embodiments, the aryl is a phenyl.

Additionally or alternatively, the siNA molecules disclosed herein may comprise in the sense strand, the antisense strand, or both a 5′-stabilized end cap of Formula (Ic):

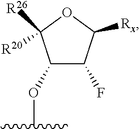

wherein Rₓ is a nucleobase, aryl, heteroaryl, or H,
R²⁶ is

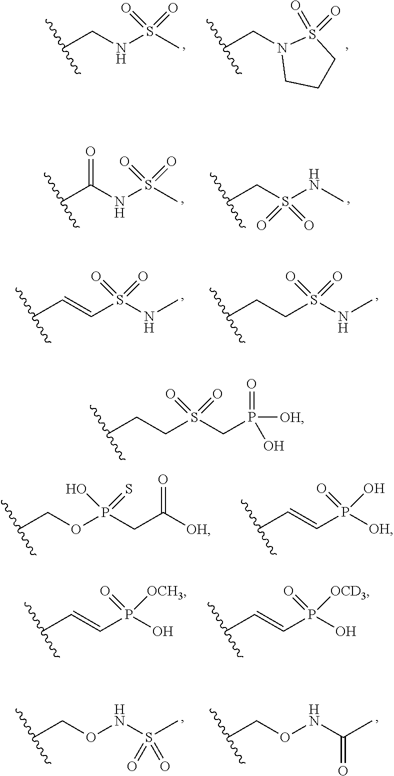

—CH═CD—Z, —CD═CH—Z, —CD-CD—Z, —(CR²¹R²²)ₙ—Z, or —(C₂—Ce alkenylene)-Z and R²⁰ is hydrogen; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4;
Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴ or —NR²³SOR²⁴; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O) R²⁵; or

R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIa):

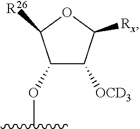

wherein Rₓ is a nucleobase, aryl, heteroaryl, or H, R²⁶ is

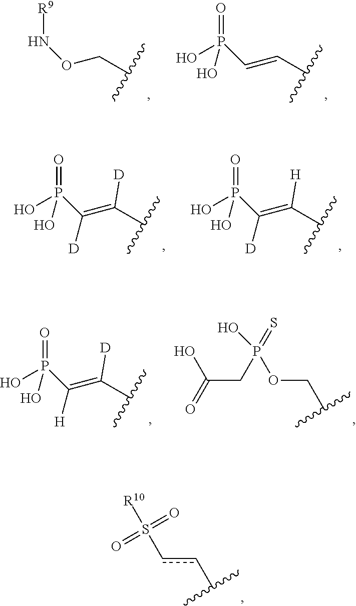

—CH₂SO₂NHCH₃, or

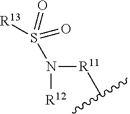

R⁹ is —SO₂CH₃ or —COCH₃, [symbol] is a double or single bond, R¹⁰=— CH₂PO₃H or —NHCH₃, R¹¹ is —CH₂— or —CO—, and R¹² is Hand R¹³ is CH; or R¹² and R¹³ together form-CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a S′-stabilized end cap of Formula (IIb);

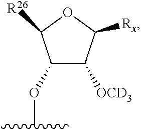

wherein Rₓ is a nucleobase, aryl, heteroaryl, or H, R²⁶ is

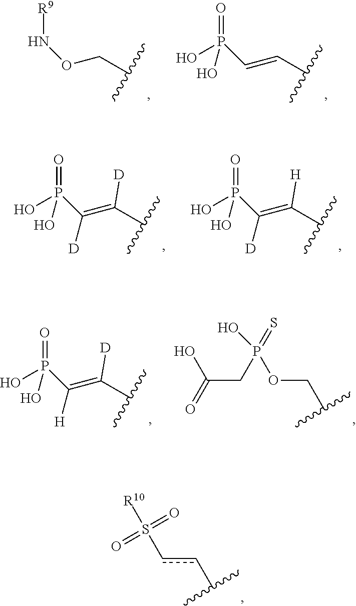

—CH₂SO₂NHCH₃, of

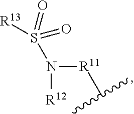

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form-CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (III):

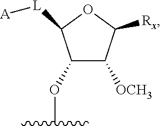

wherein Rₓ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂). In some embodiments, R¹ is an aryl. In some embodiments, the aryl is a phenyl.

Additionally or alternatively, the siNA molecules disclosed herein may comprise a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61), Formula (62), and Formula (63):

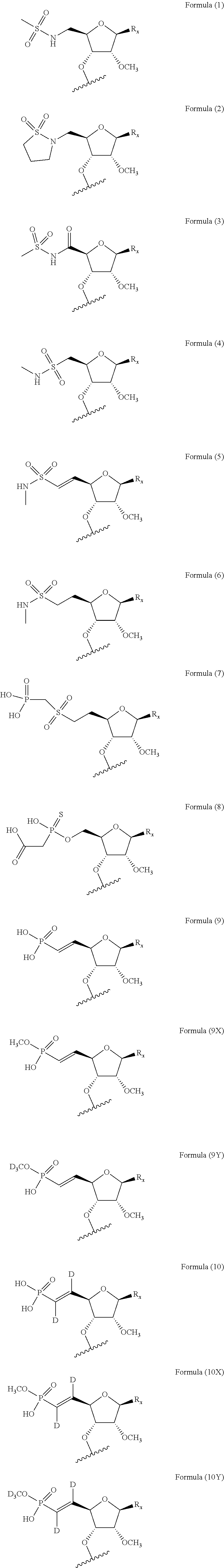

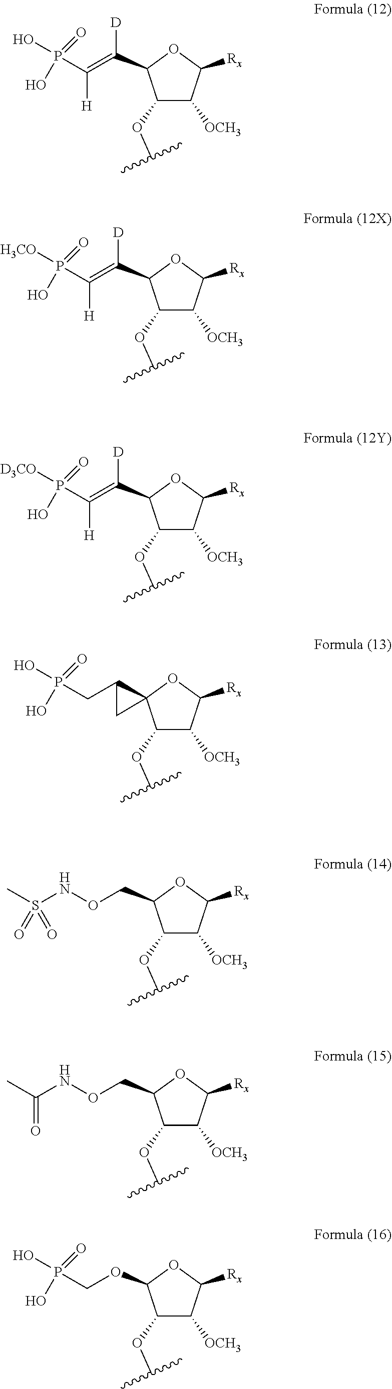

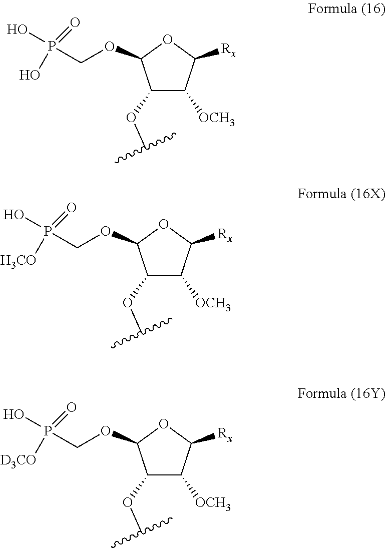

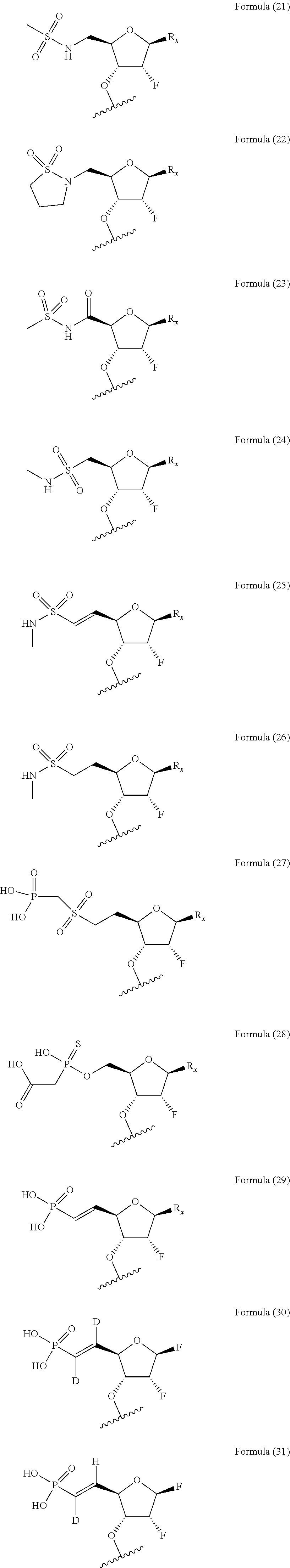

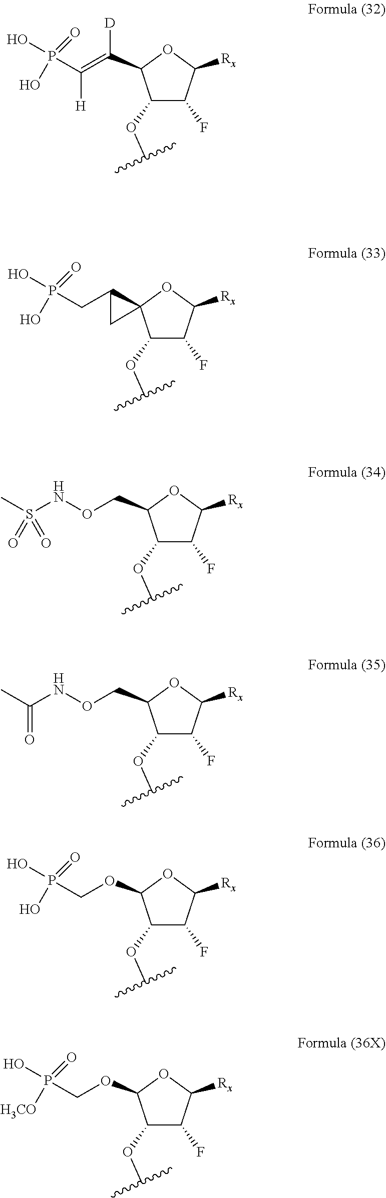

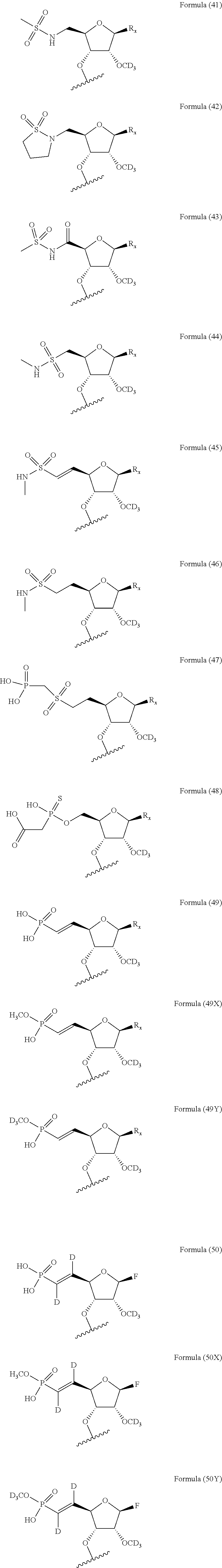

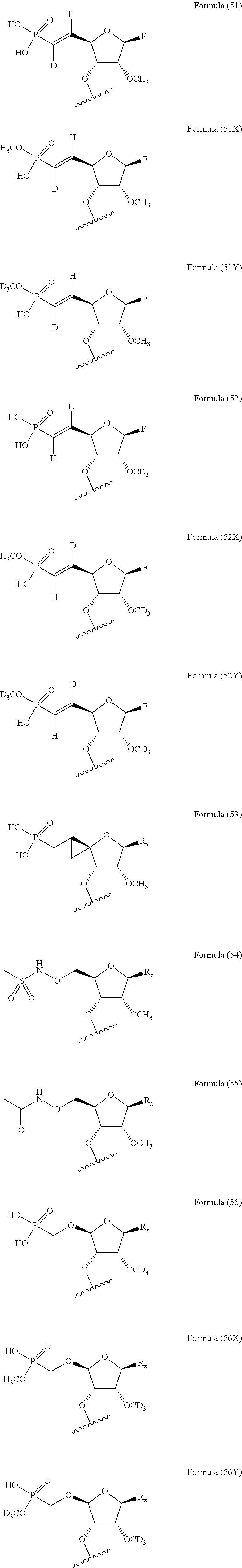

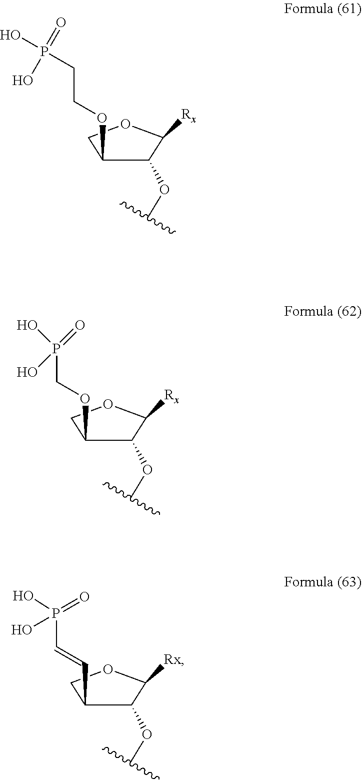

wherein Rₓ is a nucleobase, aryl, hetero aryl, or H.

In some embodiments, any of the siNA molecules disclosed herein comprise a 5′-stabilized end cap selected from the group consisting of Formula (50), Formula (50X), Formula (50Y), Formula (56), Formula (56X), Formula (56Y), Formula (61), Formula (62), and Formula (63):

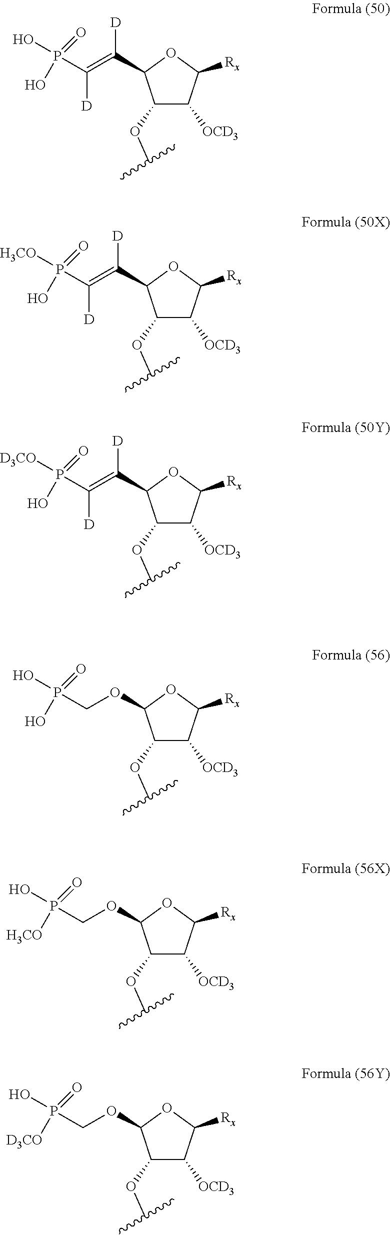

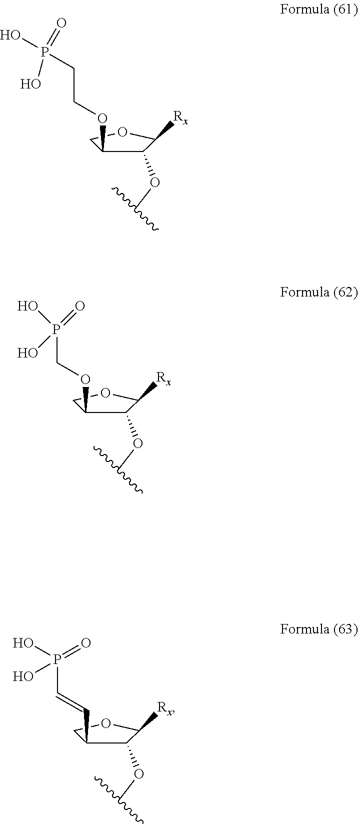

wherein Rₓ is a nucleobase, aryl, heteroaryl, or H.

In some embodiments, any of the siNA molecules disclosed herein comprise a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′:

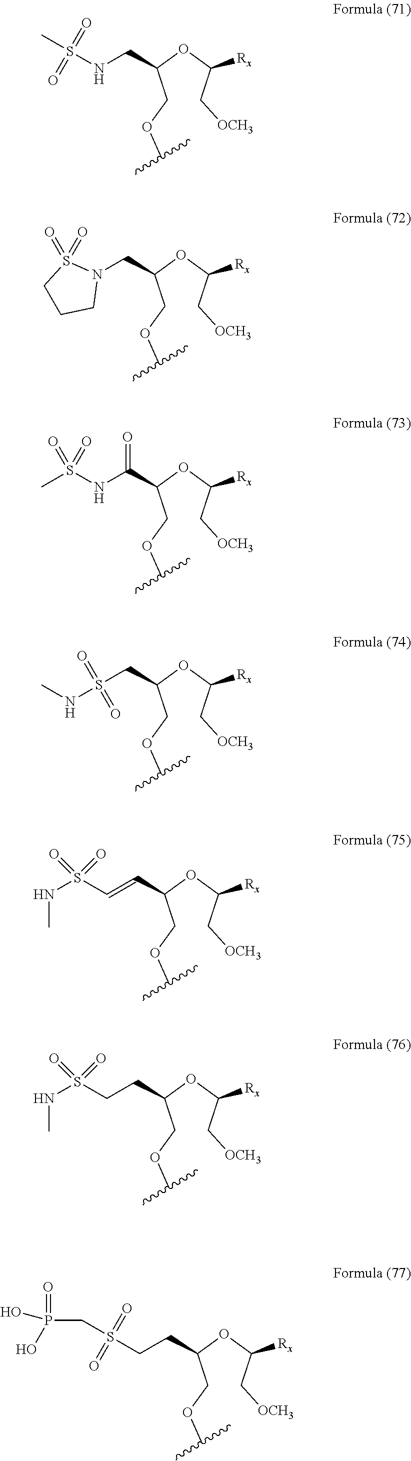

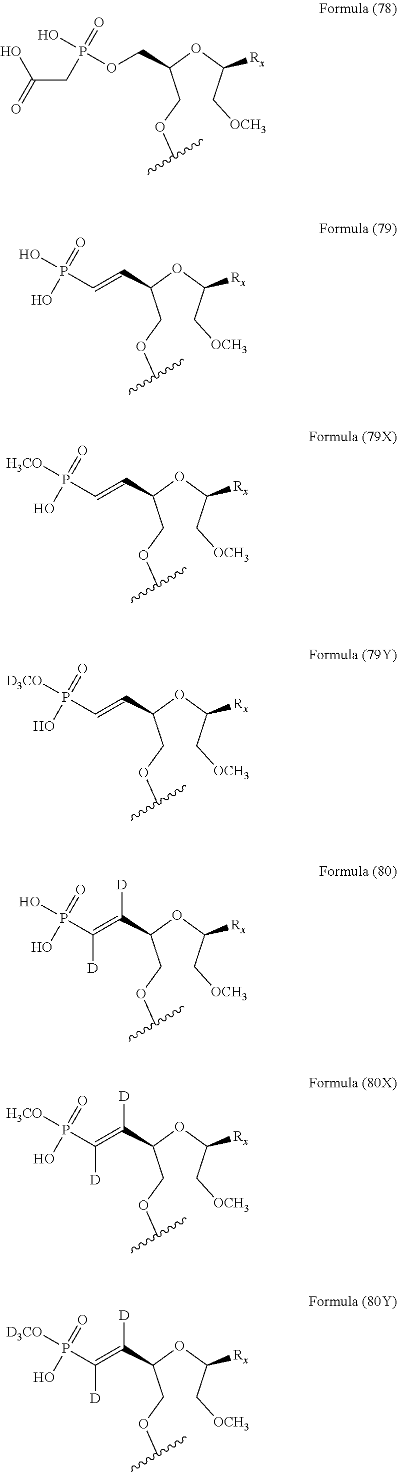

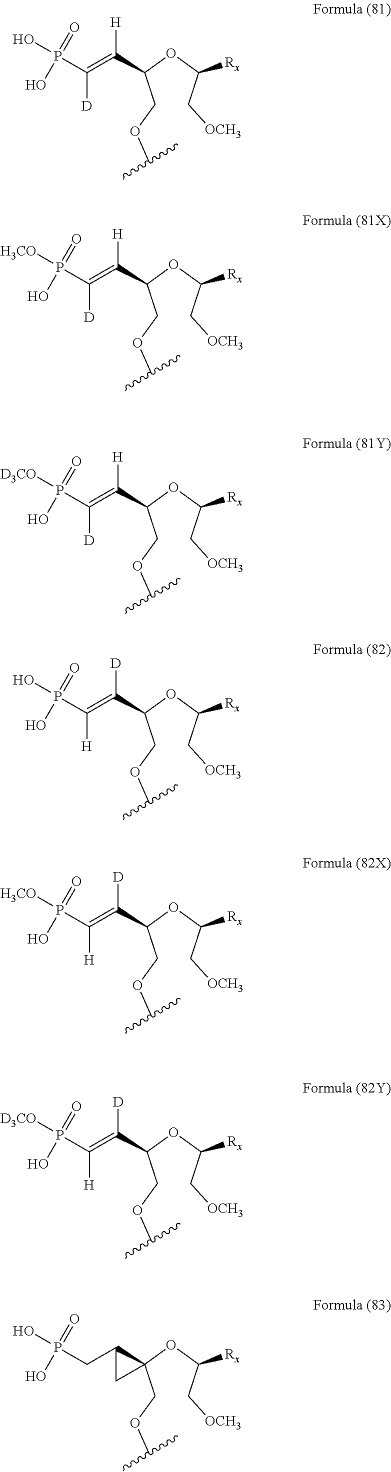

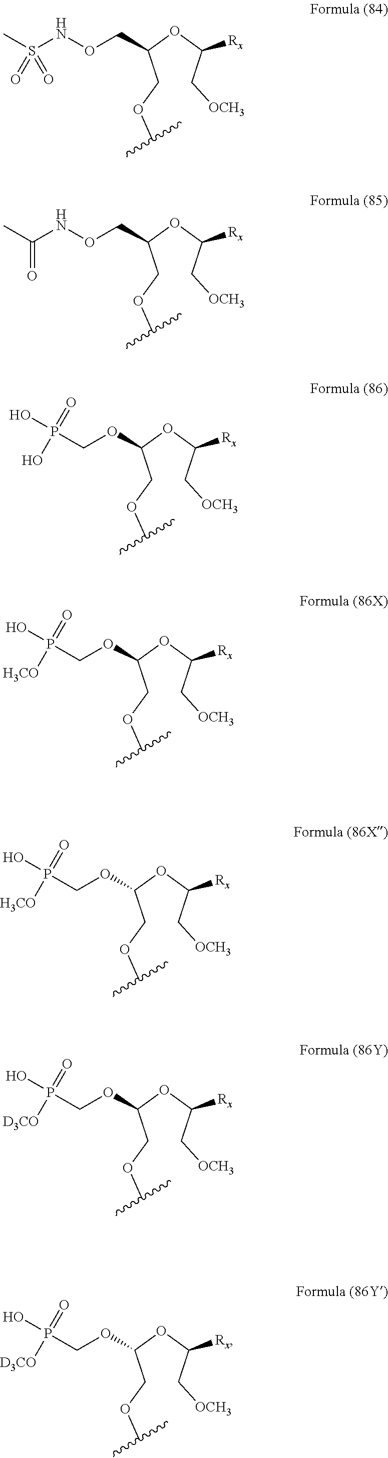

wherein Rₓ is a nucleobase, aryl, heteroaryl, or H.

In some embodiments, any of the siNA molecules disclosed herein comprise a 5′-stabilized end cap selected from the group consisting of Formula (78), Formula (79), Formula (79X), Formula (79Y), Formula (86), Formula (86X), and Formula (86X′):

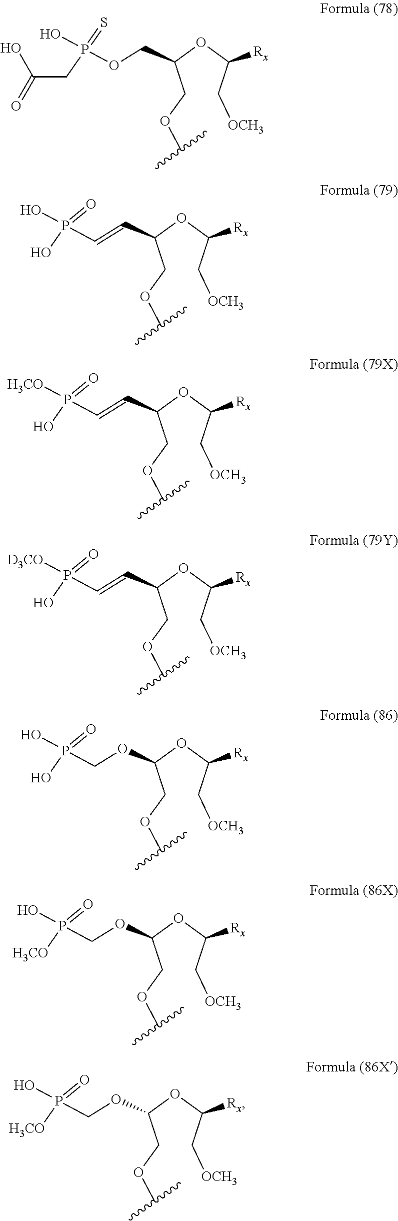

wherein Rₓ is a nucleobase, aryl, heteroaryl, or H.

In some embodiments, any of the siNA molecules disclosed herein comprise a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY):

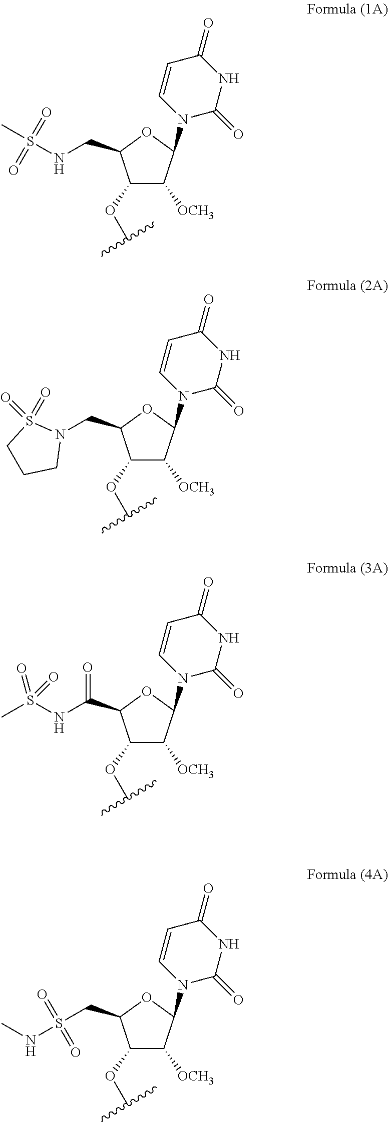 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC]

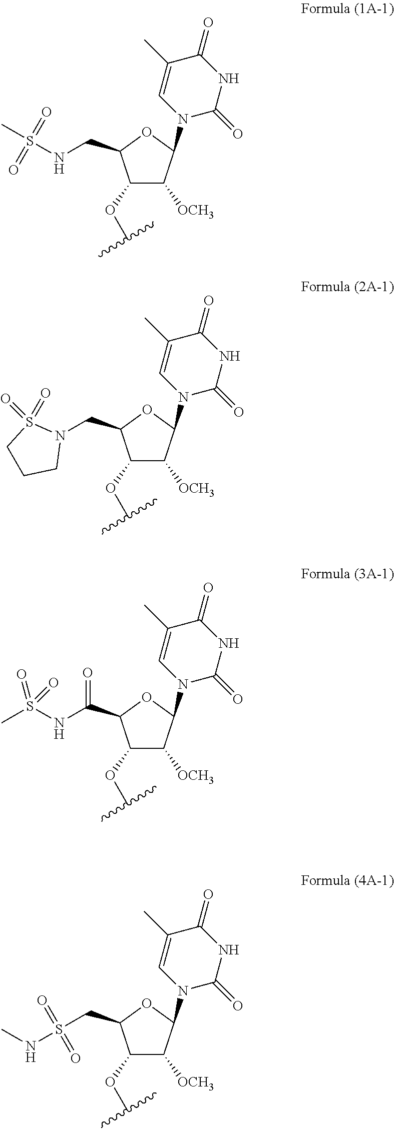 [SMILES: *O[C@H]1[C@@H](OC)[C@H](n2cc(C)c(=O)[nH]c2=O)O[C@@H]1CNS(C)(=O)=O ; *O[C@H]1[C@@H](OC)[C@H](n2cc(C)c(=O)[nH]c2=O)O[C@@H]1CN1CCCS1(=O)=O ; *O[C@H]1[C@@H](OC)[C@H](n2cc(C)c(=O)[nH]c2=O)O[C@@H]1C(=O)NS(C)(=O)=O ; *O[C@H]1[C@@H](OC)[C@H](n2cc(C)c(=O)[nH]c2=O)O[C@@H]1CS(=O)(=O)NC]

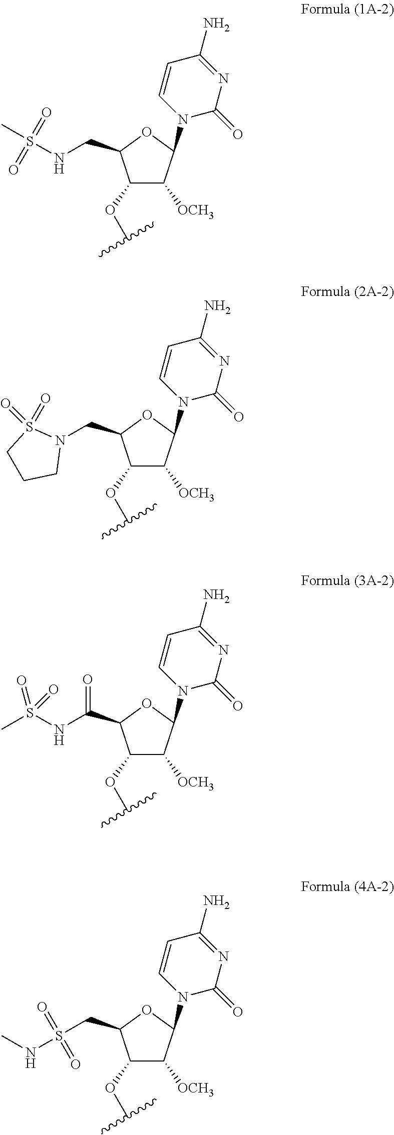 [SMILES: *O[C@H]1[C@@H](OC)[C@H](n2ccc(N)nc2=O)O[C@@H]1CNS(C)(=O)=O ; *O[C@H]1[C@@H](OC)[C@H](n2ccc(N)nc2=O)O[C@@H]1CN1CCCS1(=O)=O ; *O[C@H]1[C@@H](OC)[C@H](n2ccc(N)nc2=O)O[C@@H]1C(=O)NS(C)(=O)=O ; *O[C@H]1[C@@H](OC)[C@H](n2ccc(N)nc2=O)O[C@@H]1CS(=O)(=O)NC]

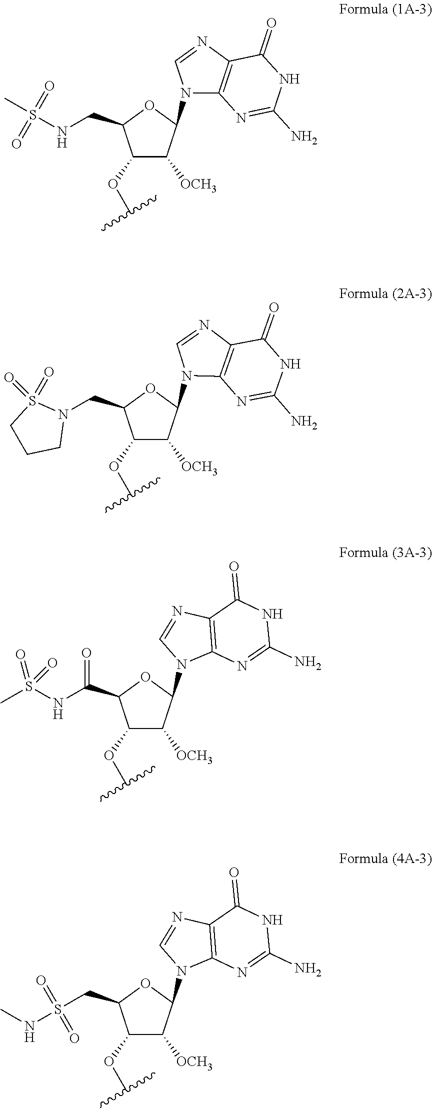 [SMILES: *O[C@H]1[C@@H](OC)[C@H](n2cnc3c(=O)[nH]c(N)nc32)O[C@@H]1CNS(C)(=O)=O ; *O[C@H]1[C@@H](OC)[C@H](n2cnc3c(=O)[nH]c(N)nc32)O[C@@H]1CN1CCCS1(=O)=O ; *O[C@H]1[C@@H](OC)[C@H](n2cnc3c(=O)[nH]c(N)nc32)O[C@@H]1C(=O)NS(C)(=O)=O ; *O[C@H]1[C@@H](OC)[C@H](n2cnc3c(=O)[nH]c(N)nc32)O[C@@H]1CS(=O)(=O)NC]

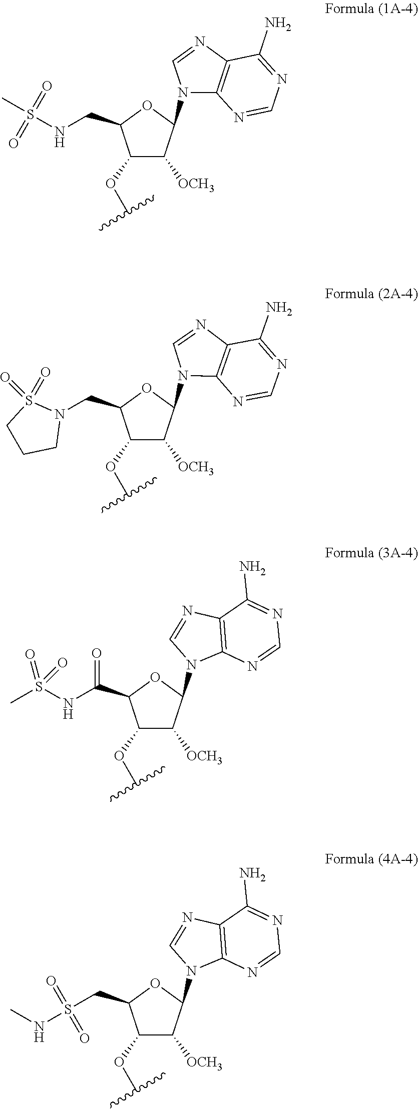 [SMILES: *O[C@H]1[C@@H](OC)[C@H](n2cnc3c(N)ncnc32)O[C@@H]1CNS(C)(=O)=O ; *O[C@H]1[C@@H](OC)[C@H](n2cnc3c(N)ncnc32)O[C@@H]1CN1CCCS1(=O)=O ; *O[C@H]1[C@@H](OC)[C@H](n2cnc3c(N)ncnc32)O[C@@H]1C(=O)NS(C)(=O)=O ; *O[C@H]1[C@@H](OC)[C@H](n2cnc3c(N)ncnc32)O[C@@H]1CS(=O)(=O)NC]

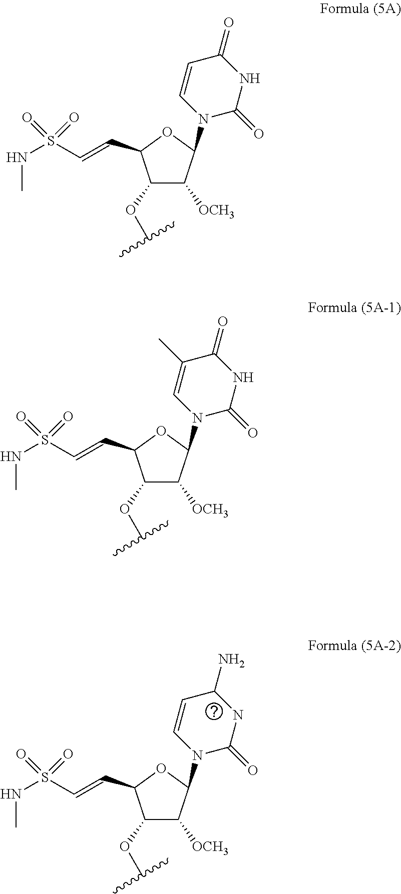 [SMILES: *O[C@H]1[C@@H](OC)[C@H](n2ccc(=O)[nH]c2=O)O[C@@H]1/C=C/S(=O)(=O)NC ; *O[C@H]1[C@@H](OC)[C@H](n2cc(C)c(=O)[nH]c2=O)O[C@@H]1/C=C/S(=O)(=O)NC ; *O[C@H]1[C@@H](OC)[C@H](N2C=CC(N)NC2=O)O[C@@H]1/C=C/S(=O)(=O)NC]

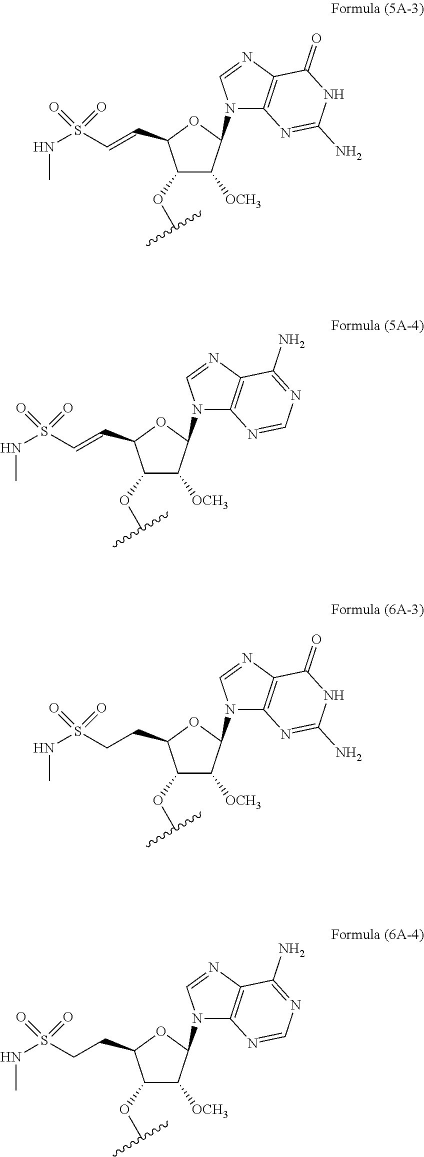 [SMILES: *O[C@H]1[C@@H](OC)[C@H](n2cnc3c(=O)[nH]c(N)nc32)O[C@@H]1/C=C/S(=O)(=O)NC ; *O[C@H]1[C@@H](OC)[C@H](n2cnc3c(N)ncnc32)O[C@@H]1/C=C/S(=O)(=O)NC ; *O[C@H]1[C@@H](OC)[C@H](n2cnc3c(=O)[nH]c(N)nc32)O[C@@H]1CCS(=O)(=O)NC ; *O[C@H]1[C@@H](OC)[C@H](n2cnc3c(N)ncnc32)O[C@@H]1CCS(=O)(=O)NC]

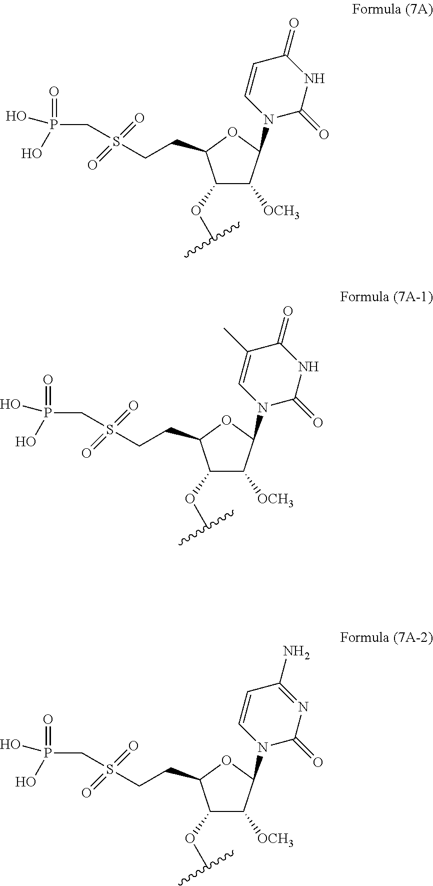 [SMILES: *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2cc(C)c(=O)[nH]c2=O)O[C@@H]1CCS(=O)(=O)CP(=O)(O)O ; *O[C@H]1[C@@H](OC)[C@H](n2ccc(N)nc2=O)O[C@@H]1CCS(=O)(=O)CP(=O)(O)O]

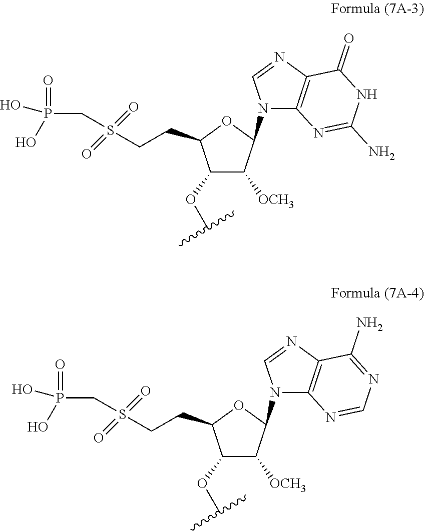 [SMILES: *O[C@H]1[C@@H](OC)[C@H](n2cnc3c(=O)[nH]c(N)nc32)O[C@@H]1CCS(=O)(=O)CP(=O)(O)O ; *O[C@H]1[C@@H](OC)[C@H](n2cnc3c(N)ncnc32)O[C@@H]1CCS(=O)(=O)CP(=O)(O)O]

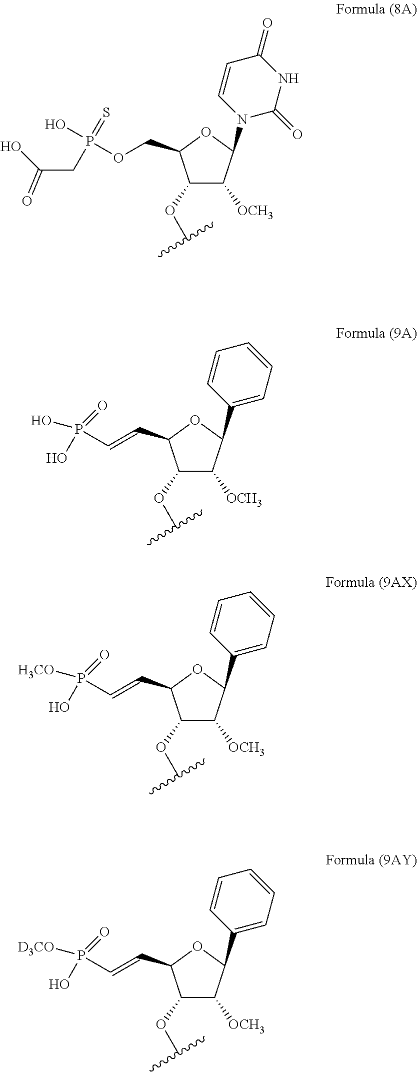

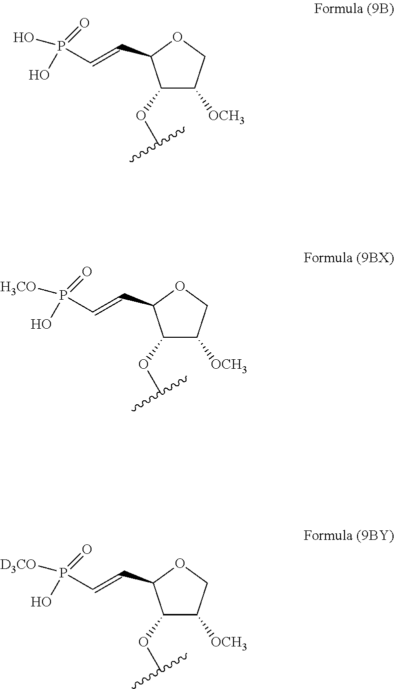

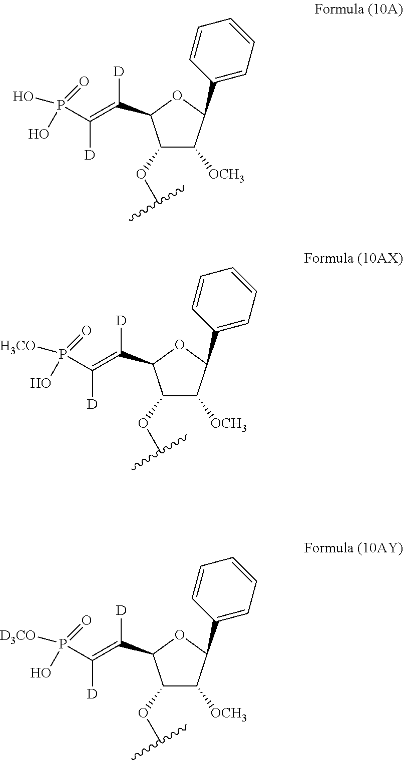

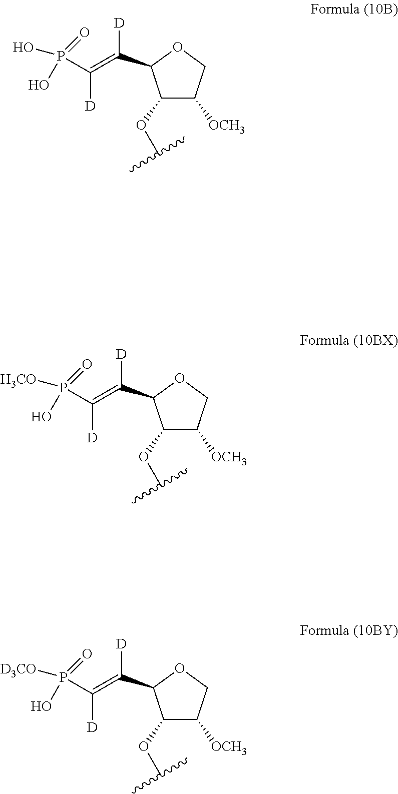

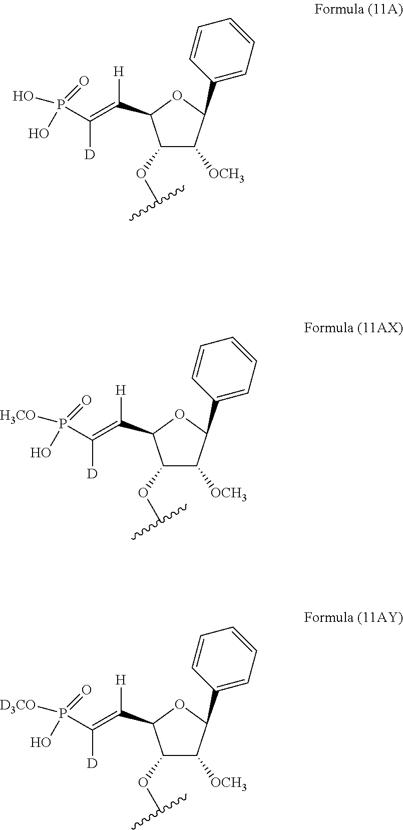

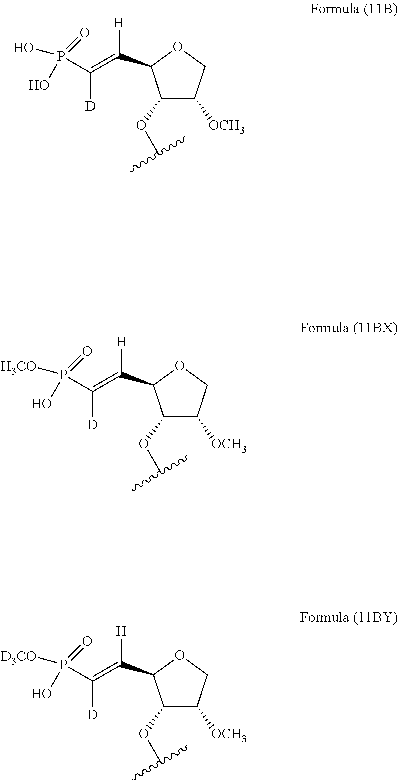

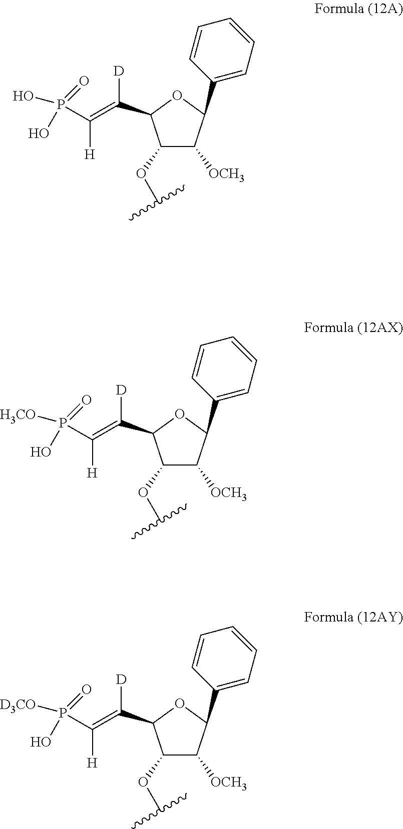

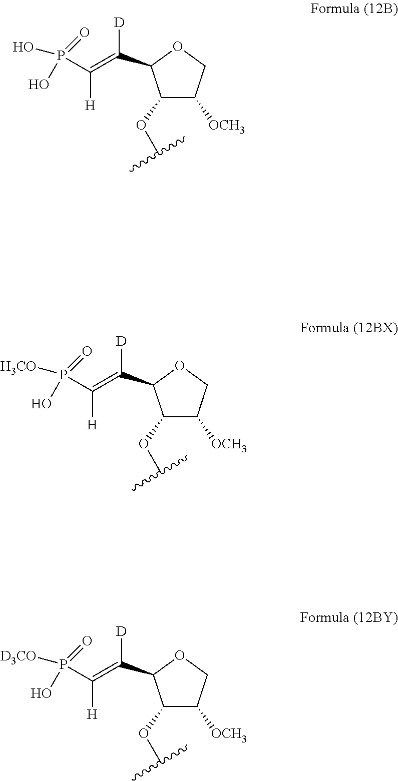

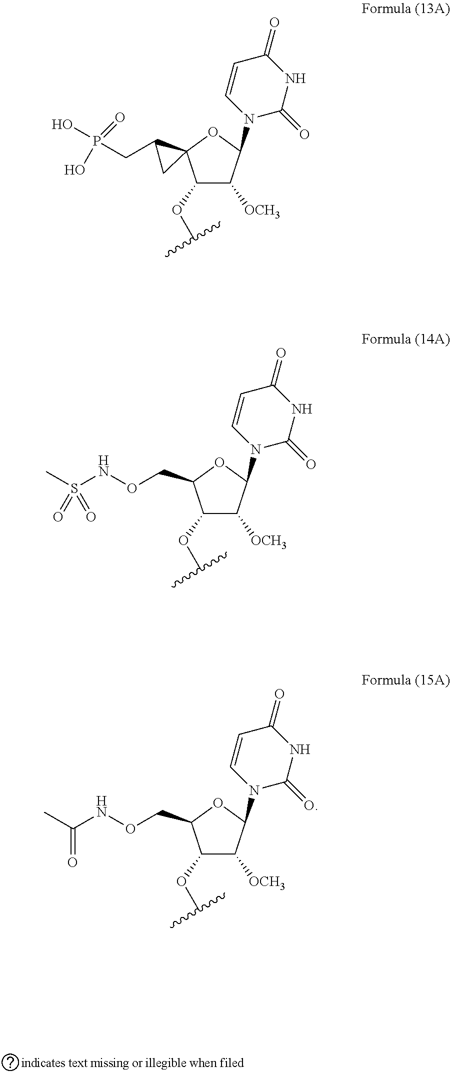

In some embodiments, any of the siNA molecules disclosed herein comprise a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

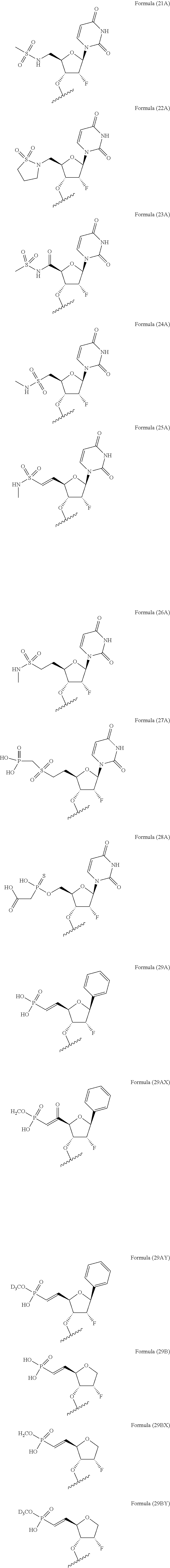

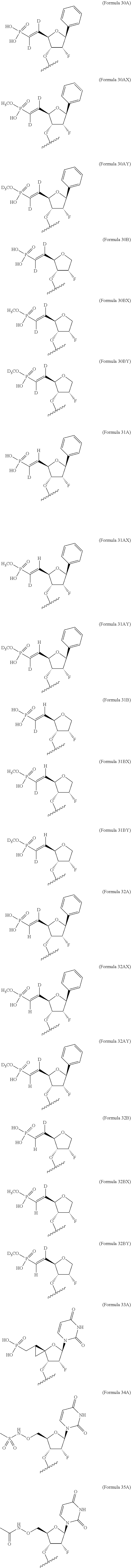

In some embodiments, any of the siNA molecules disclosed herein comprise a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′):

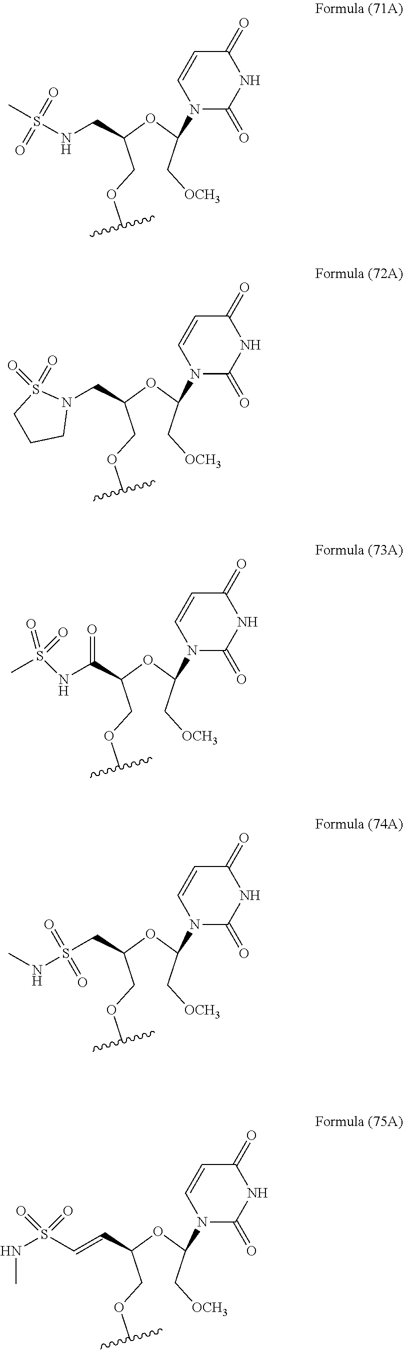 [SMILES: *OC[C@@H](CNS(C)(=O)=O)O[C@H](COC)n1ccc(=O)[nH]c1=O ; *OC[C@@H](CN1CCCS1(=O)=O)O[C@H](COC)n1ccc(=O)[nH]c1=O ; *OC[C@H](O[C@H](COC)n1ccc(=O)[nH]c1=O)C(=O)NS(C)(=O)=O ; *OC[C@@H](CS(=O)(=O)NC)O[C@H](COC)n1ccc(=O)[nH]c1=O ; *OC[C@@H](/C=C/S(=O)(=O)NC)O[C@H](COC)n1ccc(=O)[nH]c1=O]

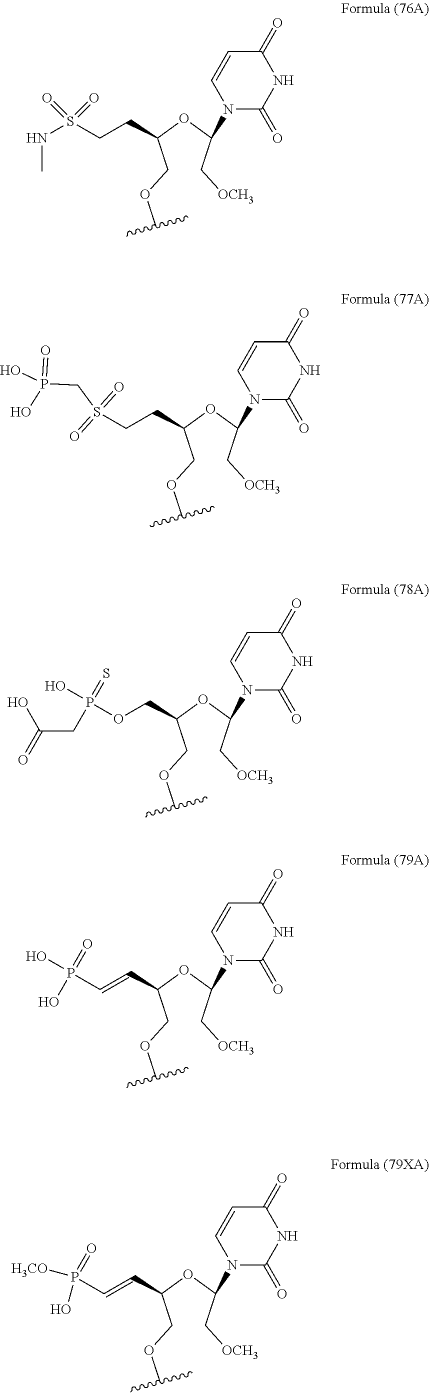 [SMILES: *OC[C@@H](CCS(=O)(=O)NC)O[C@H](COC)n1ccc(=O)[nH]c1=O ; *OC[C@@H](CCS(=O)(=O)CP(=O)(O)O)O[C@H](COC)n1ccc(=O)[nH]c1=O ; *OC[C@@H](COP(O)(=S)CC(=O)O)O[C@H](COC)n1ccc(=O)[nH]c1=O ; *OC[C@@H](/C=C/P(=O)(O)O)O[C@H](COC)n1ccc(=O)[nH]c1=O ; *OC[C@@H](/C=C/P(=O)(O)OC)O[C@H](COC)n1ccc(=O)[nH]c1=O]

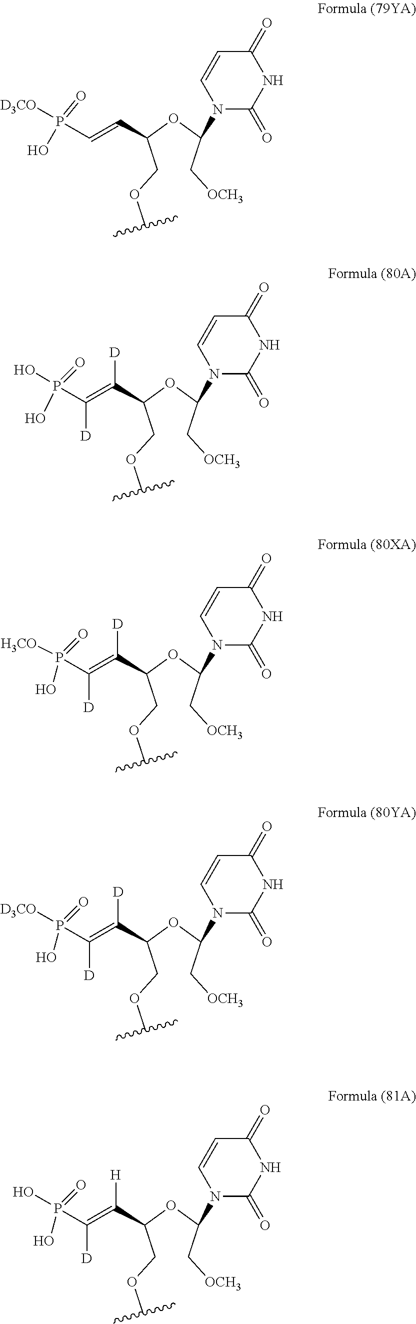

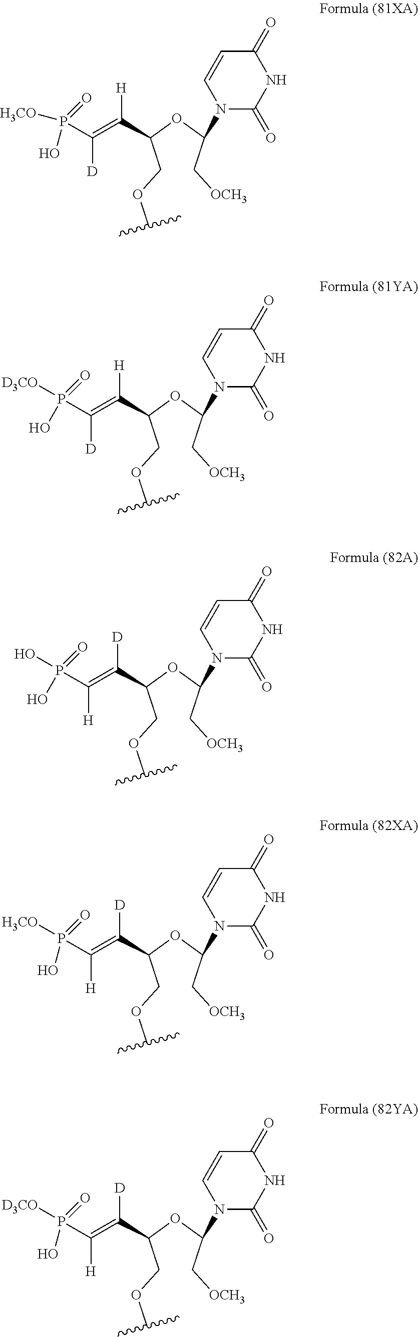

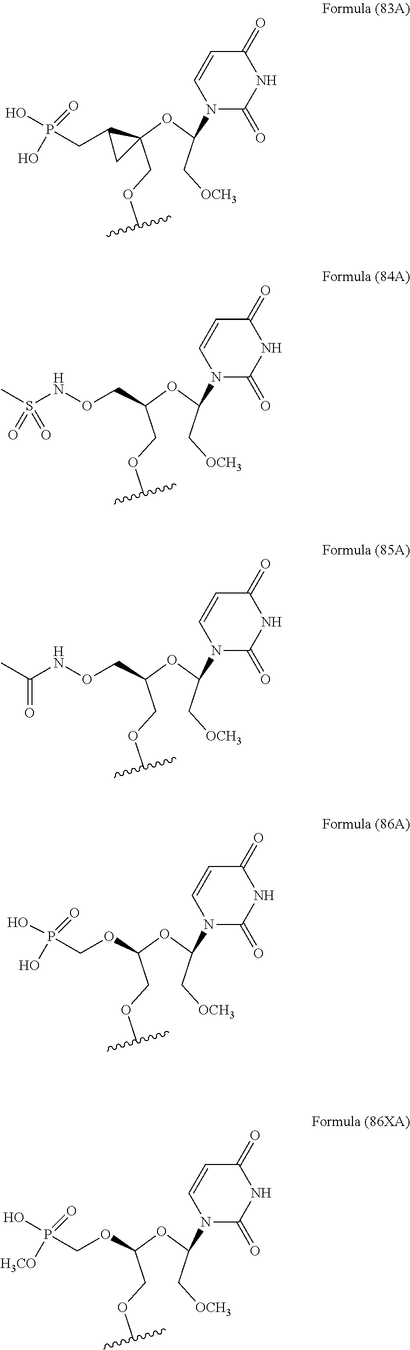

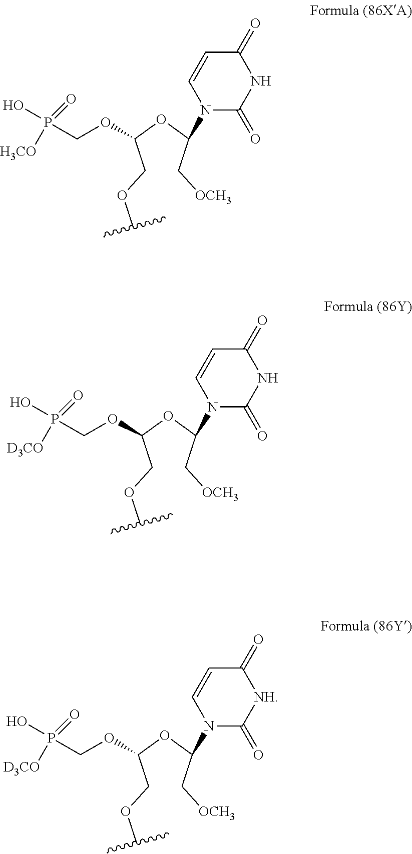

In some embodiments, any of the siNA molecules disclosed herein comprise a 5′-stabilized end cap selected from the group consisting of Formula (78A), Formula (79A), Formula (79XA), Formula (79YA), Formula (86A), Formula (86XA), and Formula (86X′A):

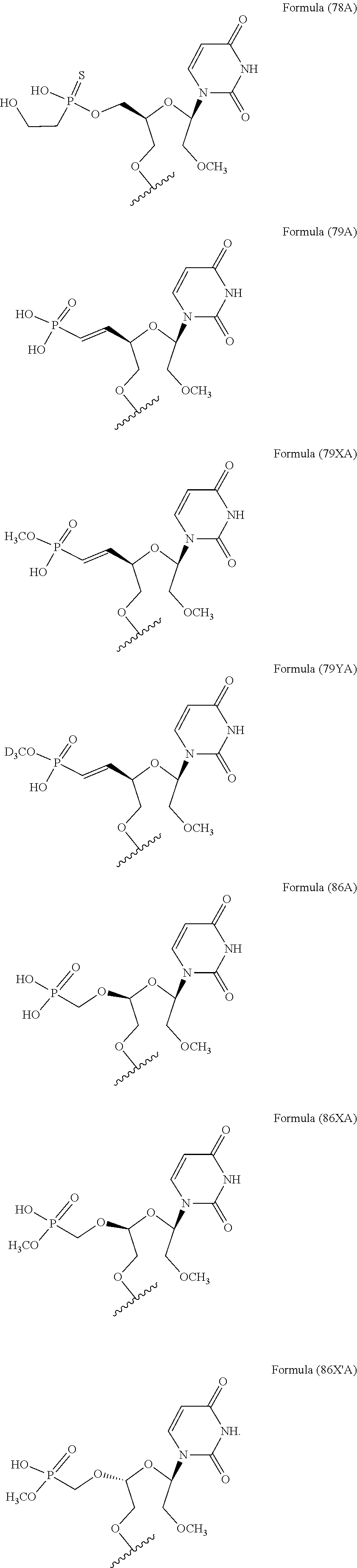

In some embodiments, the 5′-stabilized end cap is attached to the 5′ end of the antisense strand. In some embodiments, the 5′-stabilized end cap is attached to the 5′ end of the antisense strand via 1, 2, 3, 4, or 5 or more linkers. In some embodiments, the one or more linkers are independently selected from the group consisting of a phosphodiester (p or po) linker, phosphorothioate (ps) linker, mesyl phosphoramidate (Ms) linker, phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p. (PS)2-p-HEG-p, and (PS)2-p-(HEG-p)2.

As indicated above, the present disclosure provides compositions comprising any of the siNA molecules, sense strands, antisense strands, first nucleotide sequences, or second nucleotide sequences described herein. The disclosed siNA and compositions thereof can be used in the treatment of various diseases and conditions (e.g., viral diseases, liver disease, etc.).

### Linker

In some embodiments, any of the siNAs, sense strands, first nucleotide sequences, antisense strands, and/or second nucleotide sequences disclosed herein comprise 1, 2, 3, 4, 5, 6, 7, 8, 9, 10, 11, 12, 13, 14, 15, 16, 17, 18, 19, 20, 21, 22, 23 or more internucleoside linkers. In some embodiments, 1, 2, 3, 4, 5, 6, 7, 8, 9, 10 or more internucleoside linkers are independently selected from the group consisting of a phosphodiester (p or po) linker, phosphorothioate (ps) linker, mesyl phosphoramidate (Ms) linker, or phosphorodithioate linker.

In some embodiments, any of the siNAs, sense strands, first nucleotide sequences, antisense strands, and/or second nucleotide sequences disclosed herein further comprise 1, 2, 3, 4 or more linkers that attach a conjugated moiety, phosphorylation blocker, and/or 5′ end cap to the siNA, sense strand, first nucleotide sequence, antisense strand, and/or second nucleotide sequences. In some embodiments, the 1, 2, 3, 4 or more linkers are independently selected from the group consisting of a phosphodiester (p or po) linker, phosphorothioate (ps) linker, mesyl phosphoramidate (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

### Exemplary siNA

As noted above, the siNA disclosed herein may comprise a modified nucleotide at position 1 or 2 from the 3′ end of the antisense strand (i.e., N1-stabilizers). N1-stabilizing nucleotides (e.g., moe, ln, cp, mun34, bl-m, tn, 3m and bolded in Table 1) may comprise one or more of the disclosed N1-stabilizing nucleotides and the one or more N1-stabilizing nucleotides may be present in the sense strand or the antisense strand or both. Table 1 shows exemplary siNA comprising these N1-stabilizing nucleotides.

TABLE 1
siNA Comprising N1-stabilizing Nucleotides

| Name | SS/AS 5′ to 3′ |
|---|---|
| ds-siNA- | mGpsmUpsmGmGfUmGfGfAfCmUmUmCmUmCmUm |
| 001 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpsmoeGpsmA |
| | (SEQ ID NO: 2) |
| ds-siNA- | mGpsmUpsmGmGfUmGfGfAfCmUmUmCmUmCmUm |
| 002 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpsInGpsmA |
| | (SEQ ID NO: 3) |
| ds-siNA- | mGpsmUpsmGmGfUmGfGfAfCmUmUmCmUmCmUm |
| 003 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpscpGpsmA |
| | (SEQ ID NO: 4) |
| ds-siNA- | mGpsmUpsmGmGfUmGfGfAfCmUmUmCmUmCmUm |
| 004 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpsmun34GpsmA |
| | (SEQ ID NO: 5) |
| ds-siNA- | mGpsmUpsmGmGfUmGfGfAfCmUmUmCmUmCmUm |
| 005 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpsbl-mGpsmA |
| | (SEQ ID NO: 6) |
| ds-siNA- | mGpsmUpsmGmGfUmGfGfAfCmUmUmCmUmCmUm |
| 006 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCps2-mGpsmA |
| | (SEQ ID NO: 7) |
| ds-siNA- | mGpsmUpsmGmGfUmGfGfAfCmUmUmCmUmCmUm |
| 007 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCpsmGpstnA |
| | (SEQ ID NO: 8) |
| ds-siNA- | mGpsmUpsmGmGfUmGfGfAfCmUmUmCmUmCmUm |
| 008 | CmAmAmU-p-(ps)2-GalNAc4 |
| | (SEQ ID NO: 1) |
| | mApsfUpsmUmGmAfGmAmGmAmAmGmUmCfCmAf |
| | CmCmAmCps3mGpsmA |
| | (SEQ ID NO: 9) |
| ds-siNA- | mGpsmUpsmGmGfUmGfGfAfCmUmUmCmUmCmUm |
| 009 | CmAmAmU-p-(ps)2-GaINAc4 |
| | (SEQ ID NO: 1) |
| | mApsfUpsmUmGmAfGmAmGmAmAmOmUmCfCmAf |
| | CmCmAmCpsmGpsmA |
| | (SEQ ID NO: 10) |

mX = 2′-O-methyl nucleotide;
fX = 2′-fluoro nucleotide; 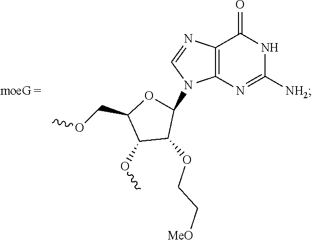 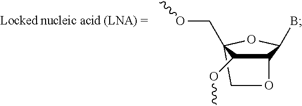
;
InG = Locked nucleic acid (LNA) G; 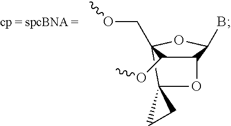
cpG = spcBNA G; 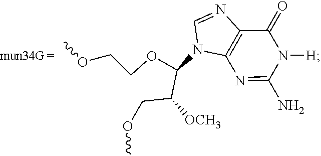
L-2′-OMe = bl-m; 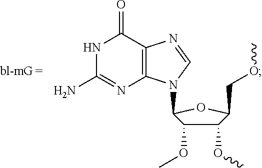 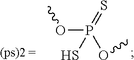 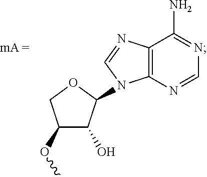 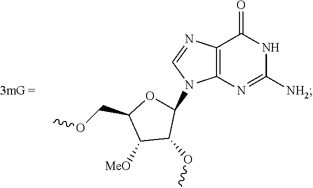
ps= phosphorothioate linkage.

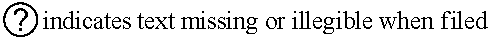

Additionally or alternatively, the disclosed siNA may also incorporate a novel nucleotide (e.g., 2′-ocp and 2′-omcp). Table 2 shows exemplary siNA comprising these nucleotides. A siNA comprising a disclosed novel nucleotides (e.g., 2′-ocp and 2′-omcp and bolded in Table 2) may comprise one or more of the disclosed novel nucleotides and the one or more novel nucleotides may be present in the sense strand or the antisense strand or both.

TABLE 2
siNA Comprising 2′-ocp and 2′-omcp Nucleotides

| Name | SS/AS 5′ to 3′ |
|---|---|
| ds-siNA- | mCpsmCpsmGmUfGmUfGfCfAm |
| 010 | CmUmUmCmGmCmUmUmCmA |
| | (SEQ ID NO: 11) |
| | 2ocpUpsfGpsmAmAfGmCmGfAm |
| | AmGmUmGmCfAmCmAfCmGmGpsm |
| | UpsmC |
| | (SEQ ID NO: 12) |
| ds-siNA- | mCpsmCpsmGmUfGmUfGfCfAm |
| 011 | CmUmUmCmGmCmUmUmCmA |
| | (SEQ ID NO: 11) |
| | 2omcpUpsfGpsmAmAfGmCmGfAm |
| | AmGmUmGmCfAmCmAfCmGmGps |
| | mUpsmC |
| | (SEQ ID NO: 13) |
| ds-siNA- | mCpsmCpsmGmUfGmUfGfCfAm |
| 012 | CmUmUmCmGmCmUmUmCmA |
| | (SEQ ID NO: 11) |
| | mUpsfGpsmAmAfG2ocpCmGfAm |
| | AmGmUmGmCfAmCmAfCmGmGpsm |
| | UpsmC |
| | (SEQ ID NO: 14) |
| ds-siNA- | mCpsmCpsmGmUfGmUfGfCfAm |
| 013 | CmUmUmCmGmCmUmUmCmA |
| | (SEQ ID NO: 11) |
| | mUpsfGpsmAmAfG2omcpCmGf |
| | AmAmGmUmGmCfAmCmAfCmGmGps |
| | mUpsmC |
| | (SEQ ID NO: 15) |
| ds-siNA- | mCpsmCpsmGmUfGmUfGfCfAm |
| 014 | CmUmUmCmGmCmUmUmCmA |
| | (SEQ ID NO: 11) |
| | mUpsfGpsmAmAfGmCmGfAmAm |
| | G2ocpUmGmCfAmCmAfCmGmGpsm |
| | UpsmC |
| | (SEQ ID NO: 16) |
| ds-siNA- | mCpsmCpsmGmUfGmUfGfCfAmCm |
| 015 | UmUmCmGmCmUmUmCmA |
| | (SEQ ID NO: 11) |
| | mUpsfGpsmAmAfGmCmGfAmAm |
| | G2omcpUmGmCfAmCmAfCmGmGps |
| | mUpsmC |
| | (SEQ ID NO: 17) |
| ds-siNA- | mCpsmCpsmGmUfGmUfGfCfAmCm |
| 016 | UmUmCmGmCmUmUmCmA |
| | (SEQ ID NO: 11) |
| | mUpsfGpsmAmAfGmCmGfAmAm |
| | GmUmG2ocpCfAmCmAfCmGmGpsm |
| | UpsmC |
| | (SEQ ID NO: 18) |
| ds-siNA- | mCpsmCpsmGmUfGmUfGfCfAm |
| 017 | CmUmUmCmGmCmUmUmCmA |
| | (SEQ ID NO: 11) |
| | mUpsfGpsmAmAfGmCmGfAmAm |
| | GmUmG2omcpCfAmCmAfCmGmGps |
| | mUpsmC |
| | (SEQ ID NO: 19) |
| ds-siNA- | mCpsmCpsmGmUfGmUfGfCfAm |
| 019 | CmUmUmCmGmCmUmUmCmA |
| | (SEQ ID NO: 11) |
| | mUpsfGpsmAmAfGmCmGfAmAm |
| | GmUmGmCfA2ocpCmAfCmGmGpsm |
| | UpsmC |
| | (SEQ ID NO: 20) |
| ds-siNA- | mCpsmCpsmGmUfGmUfGfCfAm |
| 020 | CmUmUmCmGmCmUmUmCmA |
| | (SEQ ID NO: 11) |
| | mUpsfGpsmAmAfGmCmGfAmAm |
| | GmUmGmCfA2omcpCmAfCmGmGps |
| | mUpsmC |
| | (SEQ ID NO: 21) |
| ds-siNA- | mCpsmCpsmGmUfGmUfGfCfAm |
| 021 | CmUmUmCmGmCmUmUmCmA |
| | (SEQ ID NO: 11) |
| | mUpsfGpsmAmAfGmCmGfAmAm |
| | GmUmGmCfAmCmA2ocpCmGmGps |
| | mUpsmC |
| | (SEQ ID NO: 22) |
| ds-siNA- | mCpsmCpsmGmUfGmUfGfCfAmCm |
| 022 | UmUmCmGmCmUmUmCmA |
| | (SEQ ID NO: 11) |
| | mUpsfGpsmAmAfGmCmGfAmAm |
| | GmUmGmCfAmCmA2omcpCmGmGp |
| | smUpsmC |
| | (SEQ ID NO: 23) |
| ds-siNA- | mCpsmCpsmGmUfGmUfGfCfAm |
| 023 | CmUmUmCmGmCmUmUmCmA |
| | (SEQ ID NO: 11) |
| | mUpsfGpsmAmAfGmCmGfAmAm |
| | GmUmGmCfAmCmAfCmGmGps2ocp |
| | UpsmC |
| | (SEQ ID NO: 24) |
| ds-siNA- | mCpsmCpsmGmUfGmUfGfCfAm |
| 024 | CmUmUmCmGmCmUmUmCmA |
| | (SEQ ID NO: 11) |
| | mUpsfGpsmAmAfGmCmGfAmAm |
| | GmUmGmCfAmCmAfCmGmGps2om |
| | cpUpsmC |
| | (SEQ ID NO: 25) |
| ds-siNA- | mCpsmCpsmGmUfGmUfGfCfAm |
| 025 | CmUmUmCmGmCmUmUmCmA |
| | (SEQ ID NO: 11) |
| | mUpsfGpsmAmAfGmCmGfAmAm |
| | GmUmGmCfAmCmAfCmGmGps2ocp |
| | Ups2ocpC |
| | (SEQ ID NO: 26) |
| ds-siNA- | mCpsmCpsmGmUfGmUfGfCfAm |
| 026 | CmUmUmCmGmCmUmUmCmA |
| | (SEQ ID NO: 11) |
| | mUpsfGpsmAmAfGmCmGfAmAm |
| | GmUmGmCfAmCmAfCmGmGps2om |
| | cpUps2omcpC |
| | (SEQ ID NO: 27) |

mX = 2′-O-methyl nucleotide;
fX = 2′-fluoro nucleotide;
2ocpU = 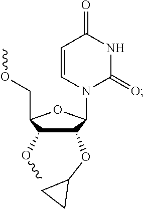
2ocpC = 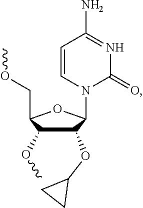
2ocpA = 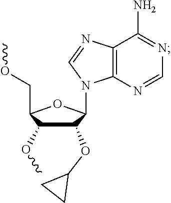
2ocpG = 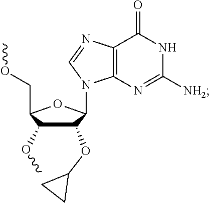
2omcpU = 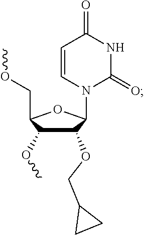
2omcpC = 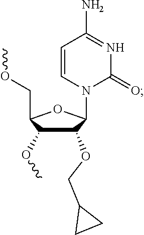
2omcpA = 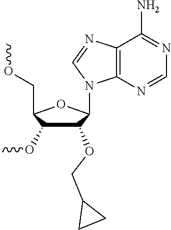
2omcpG= 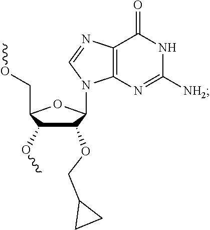
ps = phosphorothioate linkage.

Additionally or alternatively, the disclosed siNA may also incorporate a conjugated moiety. In some embodiments, conjugated moiety is galactosamine. In some embodiments, any of the siNAs disclosed herein are attached to a conjugated moiety that is galactosamine. In some embodiments, the galactosamine is N-acetylgalactosamine (GalNAc4). Table 3 shows exemplary siNA comprising these conjugated moieties in addition to novel nucleotides (e.g., 2′-ocp and 2′-omcp and bolded in the Table). In some embodiments, the siNA may comprise one or more of the disclosed conjugated moieties and the one or more conjugated moieties may be present in the sense strand or the antisense strand or both.

TABLE 3
siNA Comprising 2′-ocp and 2′-omcp Nucleotides and Conjugated Moieties

| Name | SS/AS 5′ to 3′ |
|---|---|
| ds-siNA- | 2ocpCpsmCpsmGmUfGmUfGfCfAm |
| 027 | CmUmUmCmGmCmUmUmCmA- |
| | GalNAc4 |
| | (SEQ ID NO: 28) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | 2omcpCpsmCpsmGmUfGmUfGfCf |
| 028 | AmCmUmUmCmGmCmUmUmCmA- |
| | GalNAc4 |
| | (SEQ ID NO: 30) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCps2ocpCpsmGmUfGmUfGfCfAm |
| 029 | CmUmUmCmGmCmUmUmCmA- |
| | GalNAc4 |
| | (SEQ ID NO: 31) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCps2omcpCpsmGmUfGmUfGfCfAm |
| 030 | CmUmUmCmGmCmUmUmCmA- |
| | GalNAc4 |
| | (SEQ ID NO: 32) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmG2ocpUfGmUfGfCfAm |
| 031 | CmUmUmCmGmCmUmUmCmA- |
| | GalNAc4 |
| | (SEQ ID NO: 33) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmG2omcpUfGmUfGfCfAm |
| 032 | CmUmUmCmGmCmUmUmCmA- |
| | GalNAc4 |
| | (SEQ ID NO: 34) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfG2ocpUfGfCfAm |
| 033 | CmUmUmCmGmCmUmUmCmA- |
| | GalNAc4 |
| | (SEQ ID NO: 35) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfG2omcpUfGfCf |
| 034 | AmCmUmUmCmGmCmUmUmCmA- |
| | GalNAc4 |
| | (SEQ ID NO: 36) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfA2ocp |
| 035 | CmUmUmCmGmCmUmUmCmA- |
| | GalNAc4 |
| | (SEQ ID NO: 37) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfA2omcp |
| 036 | CmUmUmCmGmCmUmUmCmA- |
| | GalNAc4 |
| | (SEQ ID NO: 38) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfAmC2 |
| 037 | ocpUmUmCmGmCmUmUmCmA- |
| | GalNAc4 |
| | (SEQ ID NO: 39) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfAmC2 |
| 038 | omcpUmUmCmGmCmUmUmCmA- |
| | GalNAc4 |
| | (SEQ ID NO: 40) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 039 | U2ocpUmCmGmCmUmUmCmA- |
| | GalNAc4 |
| | (SEQ ID NO: 41) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 040 | U2omcpUmCmGmCmUmUmCmA- |
| | GalNAc4 |
| | (SEQ ID NO: 42) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 041 | UmU2ocpCmGmCmUmUmCmA- |
| | GalNAc4 |
| | (SEQ ID NO: 43) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 042 | UmU2omcpCmGmCmUmUmCmA- |
| | GalNAc4 |
| | (SEQ ID NO: 44) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 043 | UmUmCmG2ocpCmUmUmCmA- |
| | GalNAc4 |
| | (SEQ ID NO: 45) |
| | mUps/GpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAm |
| 044 | CmUmUmCmG2omcpCmUmUmCmA- |
| | GalNAc4 |
| | (SEQ ID NO: 46) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAm |
| 045 | CmUmUmCmGmC2ocpUmUmCmA- |
| | GalNAc4 |
| | (SEQ ID NO: 47) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 046 | UmUmCmGmC2omcpUmUmCmA- |
| | GalNAc4 |
| | (SEQ ID NO: 48) |
| | mUpsfGpsmAmAfGmCmGfAmAm |
| | GmUmGmCfAmCmAfCmGmGpsmUp |
| | smC |
| | (SEQ ID NO: 29) |
| ds-siNA- | mCpsmCpsmGmUfGmUfGfCfAmCm |
| 047 | UmUmCmGmCmU2ocpUmCmA- |
| | GalNAc4 |
| | (SEQ ID NO: 49) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfAm |
| 048 | CmUmUmCmGmCmU2omcpUmCmA- |
| | GalNAc4 |
| | (SEQ ID NO: 50) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfAmCm |
| 049 | UmUmCmGmCmUmU2ocpCmA- |
| | GalNAc4 |
| | (SEQ ID NO: 51) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpamCpsmGmUfGmUfGfCfAmCm |
| 050 | UmUmCmGmCmUmU2omcpCmA- |
| | GalNAc4 |
| | (SEQ ID NO: 52) |
| | mUpsfGpsmAmAfGmCmGfAmAmGm |
| | UmGmCfAmCmAfCmGmGpsmUp |
| | smC |
| | (SEQ ID NO: 29) |
| ds-siNA- | mCpsmCpsmGmUfGmUfGfCfAm |
| 051 | CmUmUmCmGmCmUmUmCmA- |
| | GalNAc4 |
| | (SEQ ID NO: 53) |
| | mUpsfGpsmAmAfGmCmGfAmAmGm |
| | UmGmCfAmCmAfCmGmGpsmUp |
| | smC |
| | (SEQ ID NO: 29) |

mX = 2′-O-methyl nucleotide;
fX = 2′-fluoro nucleotide;
2ocpU = 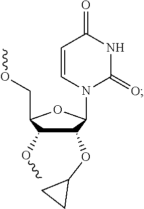
2ocpC = 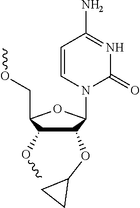
2ocpA = 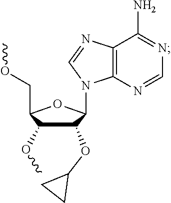
2ocpG = 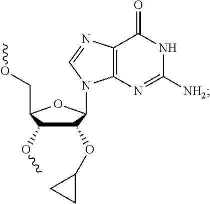
2omcpU = 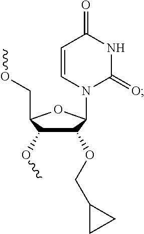
2omcpC = 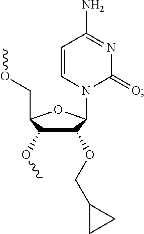
2omcpA = 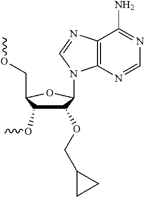
2omcpG = 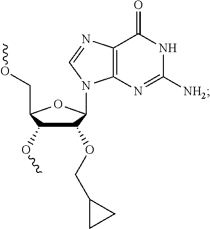
ps = phosphorothioate linkage.

Additionally or alternatively, the disclosed siNA may also incorporate alternating 2′-ocp or 2′-omcp nucleotides. Table 4 shows exemplary siNA comprising these alternating 2′-ocp or 2′-omcp nucleotides. A siNA comprising alternating 2′-ocp or 2′-omcp nucleotides (bolded in the Table) may comprise one or more alternating 2′-ocp or 2′-omcp nucleotides and the one or more alternating 2′-ocp or 2′-omcp nucleotides may be present in the sense strand or the antisense strand or both.

TABLE 4
Duplex siNA Comprising Alternating 2′-ocp or 2′-omcp Nucleotides

| Name | SS/AS (5′ to 3′) |
|---|---|
| ds-siNA-052 | mCpsmCpsmGmUfGmUfGfCfAmCm |
| | UmUmCmGmCmUmUmCmA- |
| | p-(ps)2-GalNAc4 |
| | (SEQ ID NO: 56) |
| | 2ocpUpsfGpsmAmAfGmC2ocpGf |
| | AmA2ocpGmU2ocpGmCfA2ocpC |
| | mAfC2ocpGmGps2ocpUpsmC |
| | (SEQ ID NO: 57) |
| ds-siNA-053 | mCpsmCpsmGmUfGmUfGfCfAmCm |
| | UmUmCmGmCmUmUmCmA- |
| | p-(ps)2-GalNAc4 |
| | (SEQ ID NO: 56) |
| | mUpsfGpsmAmAfGmCmGfAm |
| | A2ocpGmU2ocpGmCfA2ocpCmAfC |
| | 2ocpGmGps2ocpUpsmC |
| | (SEQ ID NO: 58) |
| ds-siNA-054 | mCpsmCpsmGmUfGmUfGfCfAm |
| | CmUmUmCmGmCmUmUmCmA- |
| | p-(ps)2-GalNAc4 (SEQ ID NO: 56) |
| | 2omcpUpsfGpsmA2omcpAfGmC |
| | 2omcpGfAmA2omcpGmU2omcpG |
| | mCfA2omcpCmAfC2omcpGmGps2omcpUpsmC |
| | (SEQ ID NO: 59) |
| ds-siNA-055 | mCpsmCpsmGmUfGmUfGfCfAm |
| | CmUmUmCmGmCmUmUmCmA- |
| | p-(ps)2-GalNAc4 |
| | (SEQ ID NO: 56) |
| | mUpsfGpsmAmAfGmCmGfA2omcp |
| | A2omcpGmU2omcpGmCfA2omcp |
| | CmAfC2omcpGmGps2omcpUpsmC |
| | (SEQ ID NO: 60) |
| ds-siNA-056 | 2ocpCpsmCps2ocpGmUfG2ocp |
| | UfGfCfA2ocpCmU2ocpUmC2ocpGm |
| | C2ocpUmU2ocpCmA-p-(ps)2-GalNAc4 |
| | (SEQ ID NO: 54) |
| | mUpsfGpsmAmAfGmCmGfAmAm |
| | GmUmGmCfAmCmAfCmGmGps |
| | mUpsmC |
| | (SEQ ID NO: 29) |
| ds-siNA-057 | 2omcpCpsmCps2omcpGmUfG2omcp |
| | UfGfCfA2omcpCmU2omcpUm |
| | C2omcpGmC2omcpUmU2omcp |
| | C2omcpA-p-(ps)2-GalNAc4 |
| | (SEQ ID NO: 55) |
| | mUpsfGpsmAmAfGmCmGfAmAm |
| | GmUmGmCfAmCmAfCmGmGps |
| | mUpsmC |
| | (SEQ ID NO: 29) |
| ds-siNA-058 | 2ocpCpsmCps2ocpGmUfG2ocp |
| | UfGfCfA2ocpCmU2ocpUmC2ocpGm |
| | C2ocpUmU2ocpCmA-p-(ps)2-GalNAc4 |
| | (SEQ ID NO: 54) |
| | 2ocpUpsfGpsmAmAfGmC2ocp |
| | GfAmA2ocpGmU2ocpGmCfA2ocpC |
| | mAfC2ocpGmGps2ocpUpsmC |
| | (SEQ ID NO: 57) |
| ds-siNA-059 | 2ocpCpsmCps2ocpGmUfG2ocpUfGfCfA |
| | 2ocpCmU2ocpUmC2ocpGm |
| | C2ocpUmU2ocpCmA- p-(ps)2-GalNAc4 |
| | (SEQ ID NO: 54) |
| | mUpsfGpsmAmAfGmCmGfAmA2ocp |
| | GmU2ocpGmCfA2ocpCmAfC |
| | 2ocpGmGps2ocpUpsmC |
| | (SEQ ID NO: 58) |
| ds-siNA-060 | 2ocpCpsmCps2ocpGmUfG2ocp |
| | UfGfCfA2ocpCmU2ocpUmC2ocpGm |
| | C2ocpUmU2ocpCmA- p-(ps)2-GalNAc4 |
| | (SEQ ID NO: 54) |
| | 2omcpUpsfGpsmA2omcpAfGmC2omcp |
| | GfAmA2omcpGmU2omcpG |
| | mCfA2omcpCmAfC2omcpGmGps2omcpUpsmC |
| | (SEQ ID NO: 59) |
| ds-siNA-061 | 2ocpCpsmCps2ocpGmUfG2ocp |
| | UfGfCfA2ocpCmU2ocpUmC2ocpGm |
| | C2ocpUmU2ocpCmA-p-(ps)2-GalNAc4 |
| | (SEQ ID NO: 54) |
| | mUpsfGpsmAmAfGmCmGfA2omcp |
| | A2omcpGmU2omcpGmCfA2omcp |
| | CmAfC2omcpGmGps2omcpUpsmC |
| | (SEQ ID NO: 60) |
| ds-siNA-062 | 2omcpCpsmCps2omcpGmUfG2omcp |
| | UfGfCfA2omcpCmU2omcpUm |
| | C2omcpGmC2omcpUmU2omcp |
| | C2omcpA-p-(ps)2-GalNAc4 |
| | (SEQ ID NO: 55) |
| | 2ocpUpsfGpsmAmAfGmC2ocpGfAm |
| | A2ocpGmU2ocpGmCfA2ocpC |
| | mAfC2ocpGmGps2ocpUpsmC |
| | (SEQ ID NO: 57) |
| ds-siNA-063 | 2omcpCpsmCps2omcpGmUfG2omcp |
| | UfGfCfA2omcpCmU2omcpUmC2omcpGm |
| | C2omcpUmU2omcpC2omcpA-p-(ps)2-GalNAc4 |
| | (SEQ ID NO: 55) |
| | mUpsfGpsmAmAfGmCmGfAm |
| | A2ocpGmU2ocpGmCfA2ocpCmAfC |
| | 2ocpGmGps2ocpUpsmC |
| | (SEQ ID NO: 58) |
| ds-siNA-064 | 2omcpCpsmCps2omcpGmUfG |
| | 2omcpUfGfCfA2omcpCmU2omcpUm |
| | C2omcpGmC2omcpUmU2omcp |
| | C2omcpA-p-(ps)2-GalNAc4 |
| | (SEQ ID NO: 55) |
| | 2omcpUpsfGpsmA2omcpAfGm |
| | C2omcpGfAmA2omcpGmU2omcpG |
| | mCfA2omcpCmAfC2omcpGmGps2omcpUpsmC |
| | (SEQ ID NO: 59) |
| ds-siNA-065 | 2omcpCpsmCps2omcpGmUfG2omcp |
| | UfGfCfA2omcpCmU2omcpUmC2omcpGm |
| | C2omcpUmU2omcpC2omcpA- p-(ps)2-GalNAc4 |
| | (SEQ ID NO: 55) |
| | mUpsfGpsmAmAfGmCmGfA2omcp |
| | A2omcpGmU2omcpGmCfA2omcp |
| | CmAfC2omcpGmGps2omcpUpsmC |
| | (SEQ ID NO: 60) |

mX = 2′-O-methyl nucleotide;
fX = 2′-fluoro nucleotide;
2ocpU = 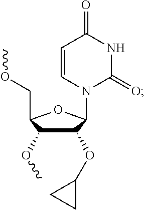
2ocpC = 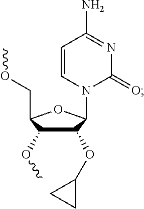
2ocpA = 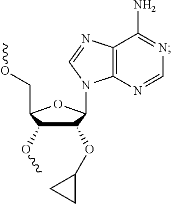
2ocpG = 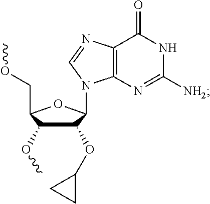
2omcpA = 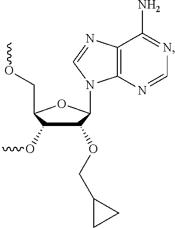
2omcpG = 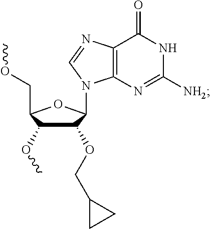
2omcpU = 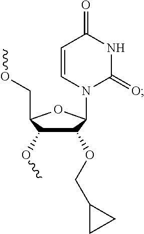
2omcpC = 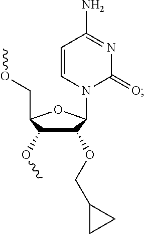
ps = phosphorothioate linkage.

Additionally or alternatively, the disclosed siNA may also incorporate 2′-ocp or 2′-omcp nucleotides at each of the 5′- and 3′-ends of the antisense strand. Table S shows exemplary siNA comprising these end-modified duplexes (bolded in the Table). An end-modified siNA may comprise 2′-ocp and/or 2′-omcp nucleotides in place of 2′-O-methyl nucleotides in the first, second, third, and/or forth position from either end of the sense strand or the antisense strand or both.

TABLE 5
siNA Comprising 2′-ocp or 2′-omcp Nucleotides at the 3′- and 5′-
ends of the Antisense Strand

| Name | SS/AS (5′to 3′) |
|---|---|
| ds-siNA-066 | mCpsmCpsmGmUfGmUfGfCfAm |
| | CmUmUmCmGmCmUmUmCmA- |
| | p-(ps)2-GalNAc4 |
| | (SEQ ID NO: 56) |
| | 2ocpUpsfGps2ocpAmAfGmCmGf |
| | AmAmGmUmGmCfAmCmAfCmG |
| | mGps2ocpUps2ocpC |
| | (SEQ ID NO: 61) |
| ds-siNA-67 | mCpsmCpsmGmUfGmUfGfCfAm |
| | CmUmUmCmGmCmUmUmCmA- |
| | p-(ps)2-GalNAc4 |
| | (SEQ ID NO: 56) |
| | 2ocpUpsfGps2ocpA2ocpAfGmCm |
| | GfAmAmGmUmGmCfAmCmAfC |
| | mG2ocpGps2ocpUps2ocpC |
| | (SEQ ID NO: 62) |
| ds-siNA-068 | mCpsmCpsmGmUfGmUfGfCfAmCm |
| | UmUmCmGmCm UmUmCmA- |
| | p-(ps)2-GalNAc4 |
| | (SEQ ID NO: 56) |
| | 2omcpUpsfGps2omcpAmAfGmCm |
| | GfAmAmGmUmGmCfAmCmAfC |
| | mGmGps2omcpUps2omcpC |
| | (SEQ ID NO: 63) |
| ds-siNA-069 | mCpsmCpsmGmUfGmUfGfCfAm |
| | CmUmUmCmGmCmUmUmCmA- |
| | p-(ps)2-GalNAc4 |
| | (SEQ ID NO: 56) |
| | 2omcpUpsfGps2omcpA2omcpAfGm |
| | CmGfAmAmGmUmGmCfAmC |
| | mAfCmG2omcpGps2omcpUps2omcpC |
| | (SEQ ID NO: 64) |

mX = 2′-O-methyl nucleotide;
fX = 2′-fluoro nucleotide;
2ocpU = 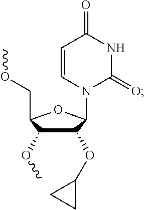
2ocpC = 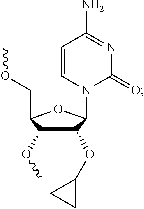
2ocpA = 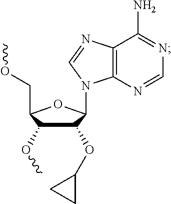
2ocpG =

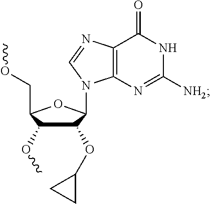

Additionally or alternatively, the disclosed siNA may also incorporate 2′-ocp or 2′-omcp nucleotides to replace one or more of the 2′O-methyl nucleotides in the 3′-overhang of the antisense strand. Table 6 shows exemplary siNA comprising these overhang-modified duplexes (bolded in the Table).

TABLE 6
siNA Comprising 2′-ocp or 2′-omcp Nucleotides in the 3′-
overhang of the Antisense Strand

| Name | SS/AS (S′ to 3′) |
|---|---|
| ds-siNA-070 | mGpsmUpsmGmGfUmGfGfAfCm |
| | UmUmCmUmCmUmCmAmAmU-p- |
| | (ps)2-GalNAc4 |
| | (SEQ ID NO: 1) |
| | mApsfUpsmUmGmAfGmAmGmAm |
| | AmGmUmCfCmAfCmCmAmCps |
| | 2ocpGpsmA |
| | (SEQ ID NO:65) |
| ds-siNA-071 | mGpsmUpsmGmGfUmGfGfAfCm |
| | UmUmCmUmCmUmCmAmAmU-p- |
| | (ps)2-GalNAc4 |
| | (SEQ ID NO. 1) |
| | mApsfUpsmUmGmAfGmAmGmAm |
| | AmGmUmCfCmAfCmCmAmCps |
| | 2omcpGpsmA |
| | (SEQ ID NO:66) |
| ds-siNA-072 | mGpsmUpsmGmGfUmGfGfAfCmUm |
| | UmCmUmCmUmCmAmAmU-p- |
| | (ps)2-GalNAc4 |
| | (SEQ ID NO. 1) |
| | mApsfUpsmUmGmAfGmAmGmAm |
| | AmGmUmCfCmAfCmCmAmCps |
| | 2ocpGps2ocpA |
| | (SEQ ID NO:67) |
| ds-siNA-073 | mGpsmUpsmGmGfUmGfGfAfCmUm |
| | UmCmUmCmUmCmAmAmU-p- |
| | (ps)2-GalNAc4 |
| | (SEQ ID NO: 1) |
| | mApsfUpsmUmGmAfGmAmGmAm |
| | AmGmUmCfCmAfCmCmAmCps |
| | 2omcpOps2omcpA |
| | (SEQ ID NO:68) |

X = 2′-O-methyl nucleotide;
fX = 2′-fluoro nucleotide;
2ocpU = 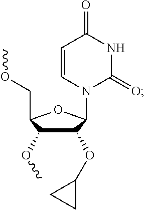
2ocpC = 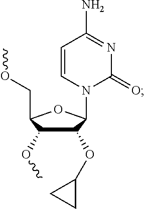
2omcpA = 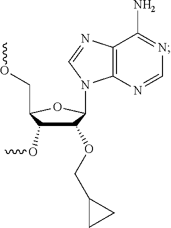
2omcpU = 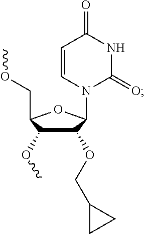
2omcpG = 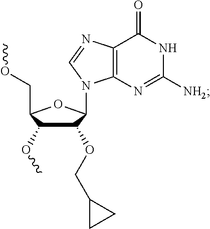
2omcpC = 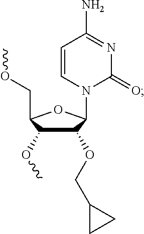
ps = phosphorothioate

Additionally or alternatively, the disclosed siNA may also incorporate 2′-ocp of 2′-omcp nucleotides to replace most of or all 2′O-methyl nucleotides. Table S shows exemplary siNA comprising these fully modified duplexes (bolded in the Table). A fully modified siNA may comprise most of all 2′-ocp and/or 2′-omcp nucleotides in place of 2′-O-methyl nucleotides in the sense strand or the antisense strand or both.

TABLE 7
Modified Duplex siNA Comprising High 2′-omcp Nucleotide Contents

| Name | SS/AS (S′ to 3″) |
|---|---|
| ds-siNA-074 | 2omcpCps2omcpCps2omcpG2omcpUfG2omcp |
| | UfGfCfA2omcpC2omcp |
| | U2omcpU2omcpC2omcpG2omcpC2omcp |
| | U2omcpU2omcpC2omcpA-p- |
| | (ps)2-GalNAc4 |
| | (SEQ ID NO: 69) |
| | mUps/GpsmAmAfGmCmGfAmAmGm |
| | UmGmCfAmCmAfCmGmGpsmU |
| | psmC |
| | (SEQ ID NO: 29) |
| ds-siNA-075 | mCpsmCpsmGmUfGmUfGfCfAmCm |
| | UmUmCmOmCmUmUmCmA-p- |
| | (ps)2-GalNAc4 |
| | (SEQ ID NO: S3) |
| | 2omcpUpsfGps2omcpA2omcpAfG |
| | 2omcpC2omcpGfA2omcpA2omcpG |
| | 2omcpU2omcpGmCfA2omcpC2omcp |
| | AfC2omcpG2omcpGps2omcpUps |
| | 2omcpC |
| | (SEQ ID NO: 70) |
| ds-siNA-076 | 2omcpCps2omcpCps2omcpG2omcp |
| | UfG2omcpUfGfCfA2omcpC2omcp |
| | U2omcpU2omcpC2omcpG2omcpC |
| | 2omcpU2omcpU2omcpC2omcpA-p- |
| | (ps)2-GalNAc4 |
| | (SEQ ID NO: 69) |
| | 2omcpUpsfGps2omcpA2omcpAfG |
| | 2omcpC2omcpGfA2omcpA2omcpG |
| | 2omcpU2omcpGmCfA2omcpC2omcp |
| | AfC2omcpGZomcpGps2omcpUps |
| | 2omcpC |
| | (SEQ ID NO: 70) |

mX = 2′-()-methyl nucleotide;
fX = 2′-fluoro nucleotide;
2ocpU = 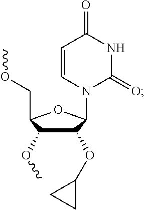
2ocpC = 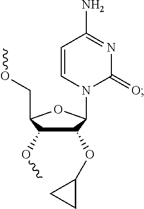
2ocpA = 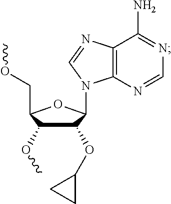
2ocpG = 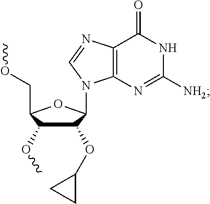
2omcpA = 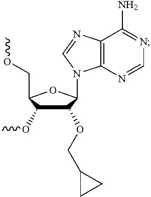
2omcpQ = 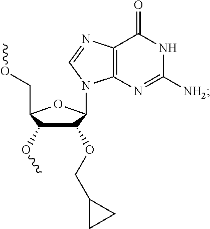
2omcpU = 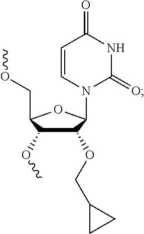
2omcpC = 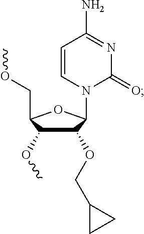
ps = phosphorothioate linkage;
X is a nucleobase (e.g. A, G, C, U or T).

Additionally or alternatively, the disclosed siNA may also incorporate a novel unlocked nucleotide monomers. These novel unlocked nucleotides may have of structure of

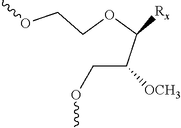

(wherein Rₓ is a nucleobase, aryl, heteroaryl, or H) or, more specifically,

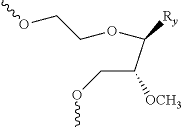

(mum34) wherein R_{y} is a nucleobase. These unlocked nucleotides are distinct from unlock nucleic acids (UNA) known in the art in which the 2′ to 3′ bond is missing

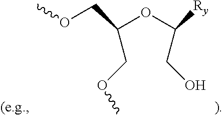

Table 7 shows exemplary siNA comprising these unlocked nucleotides (bolded in the Table). A siNA comprising a 3′,4′ UNA (e.g., mun34) may comprise one or more of the disclosed 3′,4′ UNAs and the one or more 3′,4′ UNAs may be present in the sense strand or the antisense strand or both.

TABLE 8
siNA Comprising Modified Unlocked Nucleotides and 5′
End Caps on the Antisense Strand

| Name | SS/AS (5′ to 3′) |
|---|---|
| ds-siNA-077 | mGpsmUpsmGmGfUmGfGfAfCmUmUmCm |
| | UmCmUmCmAmAmU-p-(ps)2-GalINAc4 |
| | (SEQ ID NO: 1) |
| | d2vd3ApsfUpsmUmGmAfGunAmGmAm |
| | AmGmUmCfCmAfCmCmAmCpsmGpsmA |
| | (SEQ ID NO: 71) |
| ds-siNA-078 | mGpsmUpsmGmGfUmGfGfAfCmUmUm |
| | CmUmCmUmCmAmAmU-p-(ps)2-GalNAc4 |
| | (SEQ ID NO: 1) |
| | vmApsfUpsmUmGmAfGmun34AmGmAm |
| | AmGmUmCfCmAfCmCmAmCpsmGpsmA |
| | (SEQ ID NO: 72) |
| ds-siNA-160 | mGpsmUpsmGmGfUmGfGfAfCmUmUm |
| | CmUmCmUmCmAmAmU-p-(ps)2-GalNAc4 |
| | (SEQ ID NO: 1) |
| | d2vmApsfUpsmUmGmAfGmAmGmAmAm |
| | GmUmCfCmAfCmCmAmCpsmGpsmA |
| | (SEQ ID NO: 163) |
| Control | mGpsmUpsmGmGfUmGfGfAfCmUmUmCm |
| ds-siNA-161 | UmCmUmCmAmAmU-p-(ps)2-GalNAc4 |
| | (SEQ ID NO: 1) |
| | mApsmUpsmUmGmAfGmAmGmAmAm |
| | GmUmCmCmAfCmCmAmCpsmGpsmA |
| | (SEQ ID NO: 164) |

mX = 2′-O-methyl nucleotide; d2vd3 =
vmA = 5′-vinyl phosphonate 2′-O-methyl adenosine;
d2vmA = deuterated 5′ vinyl phosphonate adenosine;
mun34 = 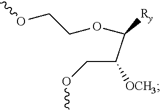
unA = unlocked adenonine;
ps= phosphorothioate linkage;
X is a nucleobase (e.g. A, G, C, U or T)

Additionally or alternatively, the disclosed siNA may also incorporate further modifications to the nucleotide monomers. These modifications include 3m, 3oh, un, mun34, as well as changes to the 2′-fluoro nucleotide pattern. Table 9 shows exemplary siNA comprising these additional modifications (bolded in the Table). In some embodiments, the siNA may comprise one or more of the disclosed modifications and the one or more disclosed modifications may be present in the sense strand or the antisense strand or both.

TABLE 9
siNA Comprising Alternative 2′-Fluoro Nucleotide Pattern

| Name | SS/AS (5′ to 3′) |
|---|---|
| ds-siNA-079 | mGpsmUpsfGmGmUmGfGfAfCmUm |
| | UfCmUmCmUmCfAmAmU-p- |
| | (ps)2-GalNAc4 |
| | (SEQ ID NO: 73) |
| | mApsfUpsmUmGmA3mGfAm |
| | GmAmAmGmUmCfCmAmCmCmAm |
| | CpsmGpsmA |
| | (SEQ ID NO: 74) |
| ds-siNA-080 | mGpsmUpsfGmGmUmGfGfAfCm |
| | UmUfCmUmCmUmCfAmAmU-p- |
| | (ps)2-GalNAc4 |
| | (SEQ ID NO: 73) |
| | mApsfUpsmUmGmA3ohGfAmGm |
| | AmAmGmUmCfCmAmCmCmAm |
| | CpsmGpsmA |
| | (SEQ ID NO: 75) |
| ds-siNA-081 | mGpsmUpsfGmGmUmGfGfAfCm |
| | UmUfCmUmCmUmCfAmAmU-p- |
| | (ps)2-GalNAc4 |
| | (SEQ ID NO: 73) |
| | mApsfUpsmUmGmAunGfAmGmAm |
| | AmGmUmCfCmAmCmCmAmC |
| | psmGpsmA |
| | (SEQ ID NO: 76) |
| ds-siNA-082 | mGpsmUpsfGmGmUmGfGfAfCmUm |
| | UfCmUmCmUmCfAmAmU-p- |
| | (ps)2-GalNAc4 |
| | (SEQ ID NO: 73) |
| | mApsfUpsmUmGmAmun34GfAmGm |
| | AmAmGmUmCfCmAmCmCm |
| | AmCpsmGpsmA |
| | (SEQ ID NO: 77) |
| ds-siNA-083 | mGpsmUpsfGmGmUmGfGfAfCmUm |
| | UfCmUmCmUmCfAmAmU-p- |
| | (ps)2-GalNAc4 |
| | (SEQ ID NO: 73) |
| | mApsmUpsmUmGmAmGfAmGmAm |
| | AmGmUmCmCmAmCmCmAm |
| | CpsmGpsmA |
| | (SEQ ID NO: 78) |
| ds-siNA-084 | mGpsmUpsfGmGmUmGfGfAfCmUm |
| | UfCmUmCmUmCfAmAmU-p- |
| | (ps)2-GalNAc4 |
| | (SEQ ID NO: 73) |
| | mApsfUpsmUmGmAmGfAmGmAm |
| | AmGmUmCfCmAmCmCmAmC |
| | psmGpsmA |
| | (SEQ ID NO: 79) |

mX = 2′-O-methyl nucleotide;
fX = 2′-fluoro nucleotide;
mun34 = 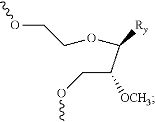
unG = unlocked guanine;
3mG = 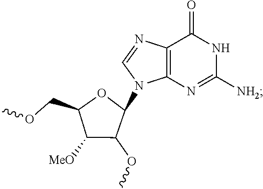
3ohG = 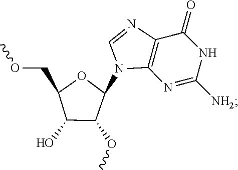
ps= phosphorothioate linkage;
X is a nucleobase (e.g. A, G, C, U or T)

Additionally or alternatively, the disclosed siNA may also incorporate further modifications to the nucleotide monomers. In some embodiments, the modifications may be 5′-cyclopropyl modifications. For example, the siNA may include 5cpr\hat{ }mA, 5 cps\hat{ }mA, 5mcpr\hat{ }mA, or 5mcps\hat{ }mA. Table 10 shows exemplary siNA comprising these additional modifications. In some embodiments, the siNA may comprise one or more of the disclosed modifications and the one or more disclosed modifications may be present in the sense strand or the antisense strand or both.

TABLE 10
siNA Comprising 5′-cyclopropyl Nucleotides on the Antisense Strand

| Name | SS/AS (5′ to 3′) |
|---|---|
| ds-siNA-085 | mGpsmUpsmGmGfUmGfGfAfCmUmUmCm |
| | UmCmUmCmAmAmU-p-(ps)2-GalNAc4 |
| | (SEQ ID NO: 1) |
| | 5cpr?mApsfUpsmUmGmAfGmAmGmAm |
| | AmGmUmCfCmAfCmCmAmCpsmGpsmA |
| | (SEQ ID NO: 80) |
| ds-siNA-086 | mGpsmUpsmGmGfUmGfGfAfCmUmUmCm |
| | UmCmUmCmAmAmU-p-(ps)2-GalNAc4 |
| | (SEQ ID NO: 1) |
| | mApsfUpsmUmGmAfGmAmGmAmAmGm |
| | UmCfCmAfCmCmAmCpsmGps5cpr?mA |
| | (SEQ ID NO: 81) |
| ds-siNA-087 | mGpsmUpsmGmGfUmGfGfAfCmUmUm |
| | CmUmCmUmCmAmAmU-p-(ps)2-GalNAc4 |
| | (SEQ ID NO: 1) |
| | 5cps?mApsfUpsmUmGmAfGmAmGmAm |
| | AmGmUmCfCmAfCmCmAmCpsmGpsmA |
| | (SEQ ID NO: 82) |
| ds-siNA-088 | inGpsmUpsmGinGfUmGfGfAfCmUmUm |
| | CmUmCmUmCmAmAmU-p-(ps)2-GalNAc4 |
| | (SEQ ID NO: 1) |
| | mApsfUpsmUmGmAfGmAmGmAmAm |
| | GmUmCfCmAfCmCmAmCpsmGps5cps?mA |
| | (SEQ ID NO: 83) |
| ds-siNA-089 | mGpsmUpsmGmGfUmGfGfAfCmUm |
| | UmCmUmCmUmCmAmAmU- |
| | p-(ps)2-GalNAc4 |
| | (SEQ ID NO: 1) |
| | 5mcpr?mApsfUpsmUmGmAfGmAmGm |
| | AmAmGmUmCfCmAfCmCmAmCpsmGpsmA |
| | (SEQ ID NO: 84) |
| ds-siNA-090 | mGpsmUpsmGmGfUmGfGfAfCmUmUm |
| | CmUmCmUmCmAmAmU-p-(ps)2-GalNAc4 |
| | (SEQ ID NO: 1) |
| | mApsfUpsmUmGmAfGmAmGmAmAmGm |
| | UmCfCmAfCmCmAmCpsmGps5mcpr?mA |
| | (SEQ ID NO: 85) |
| ds-siNA-091 | mGpsmUpsmGmGfUmGfGfAfCmUmUm |
| | CmUmCmUmCmAmAmU-p-(ps)2-GalNAc4 |
| | (SEQ ID NO: 1) |
| | 5mcps?mApsfUpsmUmGmAfGmAmGm |
| | AmAmGmUmCfCmAfCmCmAmCpsmGpsmA |
| | (SEQ ID NO: 86) |
| ds-siNA-092 | mGpsmUpsmGmGfUmGfGfAfCmUmUm |
| | CmUmCmUmCmAmAmU-p-(ps)2-GalNAc4 |
| | (SEQ ID NO: 1) |
| | m.ApsfUpsmUmGmAfGmAmGmAmAmGmUm |
| | CfCmAfCmCmAmCpsmGps5mcps?mA |
| | (SEQ ID NO: 87) |

mX = 2′-O-methyl nucleotide;
fX = 2′-fluoro nucleotide;
5mcps?mA = 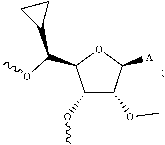
5mcpr?mA = 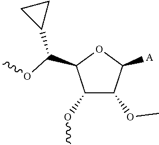
5cps?mA = 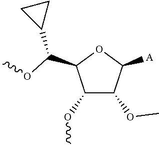
ps = phosphorothioate linkage;
X is a nucleobase (e.g. A, G, C, U or T)

Additionally or alternatively, the disclosed siNA may also incorporate further modifications to the nucleotide monomers. In some embodiments, the modifications may be 2′-F′3′-xylo modifications. For example, the siNA may include IfG, IfA, IfC, and/or IfU. Table 11 shows exemplary siNA comprising these additional modifications (bolded). In some embodiments, the siNA may comprise one or more of the disclosed modifications and the one or more disclosed modifications may be present in the sense strand or the antisense strand or both.

TABLE 11
siNA Comprising 2′-F-3′-xylo Modified Nucleotides
on the Sense or Antisense Strand

| Name | SS/AS (5′ to 3′) |
|---|---|
| ds-siNA-093 | mGpsmUpslfGmGmUmGfGfAfCmUmUfCmUm |
| | CmUmCfAmAmU-p-(ps)2-GalNAc4 |
| | (SEQ ID NO: 98) |
| | mApsfUpsmUmGmAmGfAmGmAmAmGmUm |
| | CfCmAmCmCmAmCpsmGpsmA |
| | (SEQ ID NO: 79) |
| ds-siNA-094 | mGpsmUpsfGmGmUmGIfGfAfCmUmUfCmUm |
| | CmUmCfAmAmU-p-(ps)2-GalNAc4 |
| | (SEQ ID NO: 99) |
| | mApsfUpsmUmGmAmGfAmGmAmAmGmUm |
| | CfCmAmCmCmAmCpsmGpsmA |
| | (SEQ ID NO: 79) |
| ds-siNA-095 | mGpsmUpsfGmGmUmGfGlfAfCmUmUfCmUm |
| | CmUmCfAmAmU-p-(ps)2-GalNAc4 |
| | (SEQ ID NO: 100) |
| | mApsfUpsmUmGmAmGfAmGmAmAmGmUm |
| | CfCmAmCmCmAmCpsmGpsmA |
| | (SEQ ID NO: 79) |
| ds-siNA-096 | mGpsmUpsfGmGmUmGfGfAlfCmUmUfCm |
| | UmCmUmCfAmAmU-p-(ps)2-GalNAc4 |
| | (SEQ ID NO: 101) |
| | mApsfUpsmUmGmAmGfAmGmAmAmGm |
| | UmCfCmAmCmCmAmCpsmGpsmA |
| | (SEQ ID NO: 79) |
| ds-siNA-097 | mGpsmUpsfGmGmUmGfGfAfCmUmUlfCm |
| | UmCmUmCfAmAmU-p-(ps)2-GalNAc4 |
| | (SEQ ID NO: 102) |
| | mApsfUpsmUmGmAmGfAmGmAmAmGm |
| | UmCfCmAmCmCmAmCpsmGpsmA |
| | (SEQ ID NO: 79) |
| ds-siNA-098 | mGpsmUpsfGmGmUmGfGfAfCmUmUfCm |
| | UmCmUmClfAmAmU-p-(ps)2-GalNAc4 |
| | (SEQ ID NO: 103) |
| | mApsfUpsmUmGmAmGfAmGmAmAmGm |
| | UmCfCmAmCmCmAmCpsmGpsmA |
| | (SEQ ID NO: 79) |
| ds-siNA-099 | mGpsmUpsfGmGmUmGfGfAfCmUmUfCm |
| | UmCmUmCfAmAmU-p-(ps)2-GalNAc4 |
| | (SEQ ID NO: 73) |
| | mApslfUpsmUmGmAmGfAmGmAmAmGm |
| | UmCfCmAmCmCmAmCpsmGpsmA |
| | (SEQ ID NO: 104) |
| ds-siNA-100 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmAmGIfAmGmAmAm |
| | GmUmCfCmAmCmCmAmCpsmGpsmA |
| | (SEQ ID NO: 105) |
| ds-siNA-101 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmAmGfAmGmAmAmGm |
| | UmClfCmAmCmCmAmCpsmGpsmA |
| | (SEQ ID NO: 106) |

mX = 2′-O-methyl nucleotide;
fX = 2′-fluoro nucleotide;
1fX = 2′-F-3′-xylo nucleotide 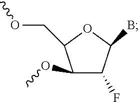
ps- phosphorothioate linkage;
X is a nucleobase (e.g. A, G, C, U ).

Additionally or alternatively, the disclosed siNA may also incorporate further modifications to the nucleotide monomers. In some embodiments, the modifications include additional 2′-F nucleotides at different positions along the antisense strand. For example, the siNA may include an additional fG, fA, fC, and/or fU. Table 12 shows exemplary siNA comprising these additional modifications (bolded). In some embodiments, the siNA may comprise one or more of the disclosed modifications and the one or more disclosed modifications may be present in the sense strand or the antisense strand or both.

TABLE 12
siNA Comprising 2′F Nucleotide ″walk″ on the Antisense Strand

| Name | SS/AS (5′ to 3′) |
|---|---|
| ds-siNA-102 | mGpsmUpsmGmUfGmCfAfCfUmUmCmGmCmUmUmCmAmCmA |
| | (SEQ ID NO: 106) |
| | mUpsfGmUmGmAgnAmGfCfGmAmAmGmUfGmCfAmCmAmCpsmU |
| | psmU |
| | (SEQ ID NO: 107) |
| ds-siNA-103 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGmAmAmGmUmCfCmAmCmCmAmCp |
| | smGpsmA |
| | (SEQ ID NO: 108) |
| ds-siNA-104 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUfGmAmGmAmGmAmAmGmUmCfCmAmCmCmAmCps |
| | mGpsmA |
| | (SEQ ID NO: 109) |
| ds-siNA-105 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGfAmGmAmGmAmAmGmUmCfCmAmCmCmAmCps |
| | mGpsmA |
| | (SEQ ID NO: 110) |
| ds-siNA-084 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGfAmGmAmAmGmUmCfCmAmCmCmAmCps |
| | mGpsmA |
| | (SEQ ID NO: 79) |
| ds-siNA-106 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGfAmAmGmUmCfCmAmCmCmAmCps |
| | mGpsmA |
| | (SEQ ID NO: 111) |
| ds-siNA-107 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGmAfAmGmUmCfCmAmCmCmAmCps |
| | mGpsmA |
| | (SEQ ID NO: 112) |
| ds-siNA-108 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGmAmAmGmUmCfCmAfCmCmAmCps |
| | mGpsmA |
| | (SEQ ID NO: 113) |
| ds-siNA-109 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGmAmAmGmUmCfCmAmCmCfAmCps |
| | mGpsmA |
| | (SEQ ID NO: 114) |
| ds-siNA-110 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGmAmGmAmAmGmUmCfCmAmCmCmAfCps |
| | mGpsmA |
| | (SEQ ID NO: 115) |

mX = 2′-O-methyl nucleotide; fX = 2′-fluoro nucleotide; gn = glycol nucleic acid (GNA); ps = phosphorothioate linkage; X is a nucleobase (e.g. A, G, C, U or T)

Additionally or alternatively, the disclosed siNA may also incorporate further modifications to the nucleotide monomers. In some embodiments, the modifications include Ganciclovir, Denvir, and 3′-ocp Nucleotides along the sense and/or antisense strand. For example, the siNA may include an ganr\hat{ }G, gans\hat{ }G, denr\hat{ }G, dens\hat{ }G, and/or a 3ocp. Table 13 shows exemplary siNA comprising these modifications (bolded). In some embodiments, the siNA may comprise one or more of the disclosed modifications and the one or more disclosed modifications may be present in the sense strand or the antisense strand or both.

TABLE 13
siNA Comprising Ganciclovir, Denvir, and 3′-ocp Nucleotides
on the Sense or Antisense Strand

| Name | SS/AS (5′ to 3′) |
|---|---|
| ds-siNA-111 | mGpsmUpsfGmGmUmGfGfAfCmUmUfCmUmCm |
| | UmCfAmAmU-p-(ps)2-GalNAc4 |
| | (SEQ ID NO: 73) |
| | mApsfUpsmUmGmAganr?GfAmGmAmAmGm |
| | UmCfCmAmCmCmAmCpsmGpsmA |
| | (SEQ ID NO: 116) |
| ds-siNA-112 | mGpsmUpsfGmGmUmGfGfAfCmUmUfCmUm |
| | CmUmCfAmAmU-p-(ps)2-GalNAc4 |
| | (SEQ ID NO: 73) |
| | mApsfUpsmUmGmAgans?GfAmGmAmAmGm |
| | UmCfCmAmCmCmAmCpsmGpsmA |
| | (SEQ ID NO: 117) |
| ds-siNA-113 | mGpsmUpsfGmGmUmGfGfAfCmUmUfCmUm |
| | CmUmCfAmAmU-p-(ps)2-GalNAc4 |
| | (SEQ ID NO: 73) |
| | mApsfUpsmUmGmAdenr?GfAmGmAmAmGm |
| | UmCfCmAmCmCmAmCpsmGpsmA |
| | (SEQ ID NO: 118) |
| ds-siNA-114 | mGpsmUpsfGmGmUmGfGfAfCmUmUfCmUm |
| | CmUmCfAmAmU-p-(ps)2-GalNAc4 |
| | (SEQ ID NO: 73) |
| | mApsfUpsmUmGmAdens?GfAmGmAmAmGm |
| | UmCfCmAmCmCmAmCpsmGpsmA |
| | (SEQ ID NO: 119) |
| ds-siNA-115 | mGpsmUpsfGmGmUmGfGfAfCmUmUfCmUm |
| | CmUmCfAmAmU-p-(ps)2-GalNAc4 |
| | (SEQ ID NO: 73) |
| | mApsfUpsmUmGmAmGfAganr?GmAmAmGm |
| | UmCfCmAmCmCmAmCpsmGpsmA |
| | (SEQ ID NO: 120) |
| ds-siNA-116 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmAmGfAgans?GmAmAm |
| | GmUmCfCmAmCmCmAmCpsmGpsmA |
| | (SEQ ID NO: 121) |
| ds-siNA-117 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmAmGfAdenr?GmAmAm |
| | GmUmCfCmAmCmCmAmCpsmGpsmA |
| | (SEQ ID NO: 122) |
| ds-siNA-118 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmAmGfAdens?GmAmAm |
| | GmUmCfCmAmCmCmAmCpsmGpsmA |
| | (SEQ ID NO: 123) |
| ds-siNA-119 | mGpsmUpsfGmGmUmGfGfAfCmUmUfCm |
| | UmCmUmCfAmAmU-p-(ps)2-GalNAc4 |
| | (SEQ ID NO: 73) |
| | mApsfUpsmUmGmA3ocpGfAmGmAmAm |
| | GmUmCfCmAmCmCmAmCpsmGpsmA |
| | (SEQ ID NO: 124) |
| ds-siNA-120 | mOpsmUpsfGmGmUmGfGfAfCmUmUf |
| | CmUmCmUmCfAmAmU-p-(ps)2-CalNAc4 |
| | (SEQ ID NO: 73) |
| | mApsfUpsmUmGmAmGfA3ocpGmAmAm |
| | GmUmCfCmAmCmCmAmCpsmGpsmA |
| | (SEQ ID NO: 125) |

mX = 2′-O-methyl nucleotide;
fX = 2′-fluoro nucleotide;
ganr?G = 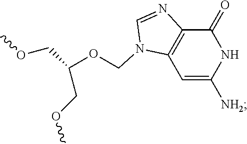
gans?G = 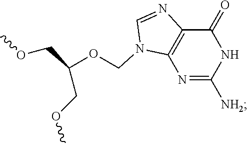
denr?G = 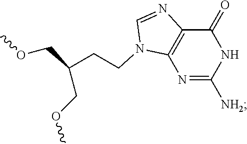
dens?G = 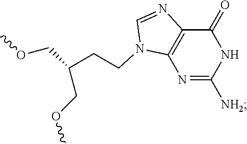
3ocpG = 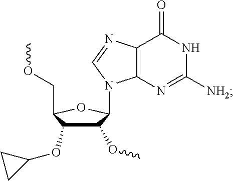
ps = phosphorothioate linkage;
X is a nucleobase (e.g. A, G, C, U or T).

Additionally or alternatively, the disclosed siNA may also incorporate further modifications to the nucleotide monomers. In some embodiments, the modifications include 2′-OMe-3′-Xylo Nucleotides along the antisense strand. For example, the siNA May include an ImG, ImG, ImG, and/or ImG. Table 14 shows exemplary siNA comprising these modifications (bolded). In some embodiments, the siNA may comprise one or more of the disclosed modifications and the one or more disclosed modifications may be present in the sense strand or the antisense strand or both.

TABLE 14
siNA Comprising 2′-OMe-3′-xylo Modified Nucleotides
on the Antisense Strand

| Name | SS/AS (S′ to 3′) |
|---|---|
| ds-siNA-084 | mGpsmUpsfGmGmUmGfGfAfCmUmUfCmUmCmUmCfAmAmU-p- |
| | (ps)2-GaINAc4 |
| | (SEQ ID NO: 73) |
| | mApsfUpsmUmGmAmGfAmGmAmAmGmUmCfCmAmCmCmAmCps |
| | mGpsmA |
| | (SEQ ID NO: 79) |
| ds-siNA-121 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGlmAmGfAmGmAmAmGmUmCfCmAmCmCmAmCp |
| | smGpsmA |
| | (SEQ ID NO: 126) |
| ds-siNA-122 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAlmGfAmGmAmAmGmUmCfCmAmCmCmAmCp |
| | smGpsmA |
| | (SEQ ID NO: 127) |
| ds-siNA-123 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGImAmGmAmAmGmUmCfCmAmCmCmAmC |
| | psmGpsmA |
| | (SEQ ID NO: 128) |
| ds-siNA-124 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGfAlmGmAmAmGmUmCfCmAmCmCmAmCp |
| | smGpsmA |
| | (SEQ ID NO: 129) |
| ds-siNA-125 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGfAmGmAmAmGmUmCfCmAmCmCmAmCpsl |
| | mGpsmA |
| | (SEQ ID NO: 130) |
| ds-siNA-126 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAlmGfAmGmAmAmGmUmCfCmAmCmCmAmCp |
| | slmGpsmA |
| | (SEQ ID NO: 131) |
| ds-siNA-127 | mGpsmUpsfGmGmUmGfGfAfCmUmUfCmUmCmUmCfAmAmU-p- |
| | (ps)2-GalNAc4 |
| | (SEQ ID NO: 73) |
| | mApsfUpsmUmGmAmGfAlmGmAmAmGmUmCfCmAmCmCmAmCp |
| | slmGpsmA |
| | (SEQ ID NO: 132) |
| ds-siNA-128 | mGpsmUpsmGmGfUmGfGfAfCmUmUmCmUmCmUmCmAmAmU- |
| | (ps)2-GalNAc4 |
| | (SEQ ID NO: 1) |
| | mApsfUpsmUmGmAfGmAmGmAmAmGmUmCfCmAfCmCmAmCps |
| | mGpslmA |
| | (SEQ ID NO: 133) |
| ds-siNA-129 | mGpsmUpsmGmGfUmGfGfAfCmUmUmCmUmCmUmCmAmAmU- |
| | (ps)2-GalNAc4 |
| | (SEQ ID NO: 1) |
| | lmApsfUpsmUmGmAfGmAmGmAmAmGmUmCfCmAfCmCmAmC |
| | psmGpsmA |
| | (SEQ ID NO: 134) |
| ds-siNA-130 | mGpsmUpsmGmGfUmGfGfAfCmUmUmCmUmCmUmCmAmAmU- |
| | (ps)2-GalNAc4 |
| | (SEQ ID NO: 1) |
| | lmApsfUpsmUmGmAfGmAmGmAmAmGmUmCfCmAfCmCmAmCps |
| | mGlpsmA |
| | (SEQ ID NO: 135) |
| ds-siNA-131 | mGpsmUpsmGmGfUmGfGfAfCmUmUmCmUmCmUmCmAmAmU- |
| | (ps)2-GalNAc4 |
| | (SEQ ID NO: 1) |
| | mApsfUpsmUmGmAfGmAmGmAmAmGmUmCfCmAfCmCmAmCpsl |
| | mGpsmA |
| | (SEQ ID NO: 136) |

mX = 2′-O-methyl nucleotide; fX = 2′-fluoro nucleotide; ImX = 2′-OMe-3′-Xylo; ps = phosphorothioate linkage; X is a nucleobase (e.g. A, G, C, U or T).

Additionally or alternatively, the disclosed siNA may also incorporate further modifications to the nucleotide monomers. In some embodiments, the modifications include 2′-ocp, 2′-omcp, and/or 5′-vinyl phosphonate 2′-O-methyl Nucleotides along the antisense strand. For example, the siNA may include an 2ocpA, 2ocpC, 2ocpG, 2ocpU, 2omcpA, 2omcpC, 2omcpG, 2omcpU, and/or vmU. Table 15 shows exemplary siNA comprising these modifications (bolded). In some embodiments, the siNA may comprise one or more of the disclosed modifications and the one or more disclosed modifications may be present in the sense strand or the antisense strand or both.

TABLE 15
siNA Comprising 2′-ocp, 2′-omcp, and vmX Nucleotides

| Name | SS/AS (5′ to 3′) |
|---|---|
| ds-siNA-132 | mGpsmUpsmGmGfUmGfGfAfCmUm |
| | UmCmUmCmUmCmAmAmU-p- |
| | (ps)2-GalNAc4 |
| | (SEQ ID NO: 1) |
| | mApsfUpsmUmGmAfGmAmGmAm |
| | AmGmUmCfCmAfCmCmAmCps |
| | mGps2ocpA |
| | (SEQ ID NO: 137) |
| ds-siNA-133 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2ocpApsfUpsmUmGmAfGmAmGmAm |
| | AmGmUmCfCmAfCmCmAmC |
| | psmGpsmA |
| | (SEQ ID NO: 138) |
| ds-siNA-134 | mGpsmUpsmGmGfUmGfGfAfCmUm |
| | UmCmUmCmUmCmAmAmU-p- |
| | (ps)2-GalNAc4 |
| | (SEQ ID NO: 1) |
| | mApsfUpsmUmGmAfGmAmGmAm |
| | AmGmUmCfCmAfCmCmAmCps |
| | mGps2omcpA |
| | (SEQ ID NO: 139) |
| ds-siNA-135 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2omcpApsfUpsmUmGmAfGmAmGm |
| | AmAmGmUmCfCmAfCmCmAm |
| | CpsmGpsmA |
| | (SEQ ID NO: 140) |
| ds-siNA-136 | mGpsmUpsmGmGfUmGfGfAfCm |
| | UmUmCmUmCmUmCmAmAmU-p- |
| | (ps)2-GalNAc4 |
| | (SEQ ID NO: 1) |
| | 2ocpApsfUpsmUmGmAfGmAmGm |
| | AmAmGmUmCfCmAfCmCmAmC |
| | psmGps2ocpA |
| | (SEQ ID NO: 141) |
| ds-siNA-137 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2omcpApsfUpsmUmGmAfGmAmGm |
| | AmAmGmUmCfCmAfCmCmAm |
| | CpsmGps2omcpA |
| | (SEQ ID NO: 142) |
| ds-siNA-138 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2ocpApsfUpsmUmGmAfGmAmGm |
| | AmAmGmUmCfCmAfCmCmAmC |
| | ps2ocpGps2ocpA |
| | (SEQ ID NO: 143) |
| ds-siNA-139 | mGpsmUpsmGmGfUmGfGfAfCmUm |
| | UmCmUmCmUmCmAmAmU-p- |
| | (ps)2-GalNAc4 |
| | (SEQ ID NO: 1) |
| | mApsfUpsmUmGmAfGmAmG2ocpAm |
| | A2ocpGmU2ocpCfC2ocpAfC2o |
| | cpCmA2ocpCpsmGps2ocpA |
| | (SEQ ID NO: 144) |
| ds-siNA-140 | mGpsmUpsmGmGfUmGfGfAfCmUm |
| | UmCmUmCmUmCmAmAmU-p- |
| | (ps)2-GalNAc4 |
| | (SEQ ID NO: 1) |
| | mApsfUpsmUmGmAfGmAmG2omcp |
| | AmA2omcpGmU2omcpCfC2om |
| | cpAfC2omcpCmA2omcpCpsmGps2omcpA |
| | (SEQ ID NO: 145) |
| ds-siNA-141 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2ocpApsfUps2ocpUmG2ocpAfG2ocpAm |
| | G2ocpAmA2ocpGmU2ocpCf |
| | C2ocpAfC2ocpCmA2ocpCpsmGps2ocpA |
| | (SEQ ID NO: 146) |
| ds-siNA-142 | mGpsmUpsmGmGfUmGfGfAfCmUm |
| | UmCmUmCmUmCmAmAmU-p- |
| | (ps)2-GalNAc4 |
| | (SEQ ID NO: 1) |
| | 2omcpApsfUps2omcpUmG2omcpAfG2 |
| | omcpAmG2omcpAmA2omcpG |
| | mU2omcpCfC2omcpAfC2omcpCmA |
| | 2omcpCpsmGps2omcpA |
| | (SEQ ID NO: 147) |
| ds-siNA-143 | mCpsmCpsmGmUfGmUfGfCfAmCm |
| | UmUmCmGmCmUmUmCmA-p- |
| | (ps)2-GalNAc4 |
| | (SEQ ID NO: 56) |
| | vmUpsfGpsmAmAfGmCmGfAm |
| | AmGmUmGmCfAmCmAfCmGmGps |
| | mUpsmC |
| | (SEQ ID NO: 148) |
| ds-SINA-144 | mCpsm:CpsmGmUfGmUfGfCfAm |
| | CmUmUmCmGmCmUmUmCmA-p- |
| | (ps)2-GalNAc4 |
| | (SEQ ID NO: 56) |
| | vmUpsfGpsmAmAfGmCmGfAmAm |
| | GmUmGmCfAmCmAfCmGmGps |
| | mUps2ocpC |
| | (SEQ ID NO: 149) |
| ds-SINA-145 | mCpsmCpsmGmUfGmUfGfCfAm |
| | CmUmUmCmGmCmUmUmCmA-p- |
| | (ps)2-GalNAc4 |
| | (SEQ ID NO: 56) |
| | vmUpsfGpsmAmAfGmCmGfAmAm |
| | GmUmGmCfAmCmAfCmGmGps |
| | mUps2omcpC |
| | (SEQ ID NO: 150) |
| ds-SINA-146 | mCpsmCpsmGmUfGmUfGfCfAmCm |
| | UmUmCmGmCmUmUmCmA-p- |
| | (ps)2-GalNAc4 |
| | (SEQ ID NO: 56) |
| | mApsfUpsmUmGmAmGfAmGmAm |
| | AmGmUmCfCmAmCmCmAmCps |
| | mGps2ocpA |
| | (SEQ ID NO: 151) |

mX = 2′-O-methyl nucleotide;
fX = 2′-fluoro nucleotide;
2ocpU = 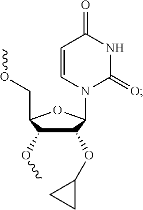
2ocpC = 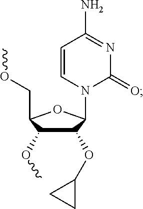
2ocpA = 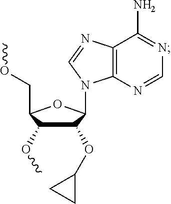
2ocpG = 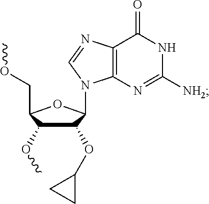
2omcpA = 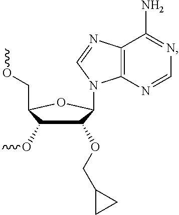
2omcpG = 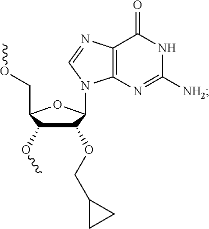
2omcpU = 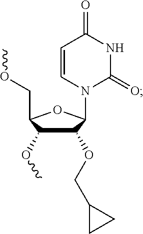
2omcpC = 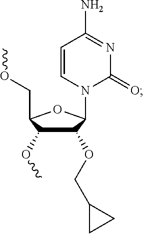
vmX = 5′-vinyl phosphonate 2′-O-methyl nucleotide;
ps = phosphorothioate linkage;
X is a nucleobase (e.g. A, G, C, U or T)

Additionally or alternatively, the disclosed siNA may also incorporate further modifications to the nucleotide monomers. In some embodiments, the modifications include vinyl phosphate 5′ end caps such as vmU and/or G analog nucleotides such as dens/G and mun12G along the antisense strand. Table 16 shows exemplary siNA comprising these modifications (bolded). In some embodiments, the siNA may comprise one or more of the disclosed modifications and the one or more disclosed modifications may be present in the sense strand or the antisense strand or both.

TABLE 16
siRNA comprising G analogs and vinyl phosphate 5′ end-caps

| Name | SS/AS 5′ to 3′ |
|---|---|
| ds-siNA- | mCpsmCpsmGmUfGmUfGfCfAmCm |
| 147 | UmUmCmGmCmUmUmCmA-p- |
| | (ps)2-GalNAc4 |
| | (SEQ ID NO: 56) |
| | vmUpsfGpsmAmAfGmCdens\hat{ }GfAm |
| | AmGmUmGmCfAmCmAfCmGmGp |
| | smUpsmC |
| | (SEQ ID NO: 153) |
| ds-siNA- | mCpsmCpsmGmUfGmUfGfCfAm |
| 148 | CmUmUmCmGmCmUmUmCmA-p- |
| | (ps)2-GalNAc4 |
| | (SEQ ID NO: 56) |
| | vmUpsfGpsmAmAfGmCmun12GfAm |
| | AmGmUmGmCfAmCmAfCmGmGp |
| | psmUpsmC |
| | (SEQ ID NO: 154) |
| ds-siRN- | mCpsmCpsmOmUfOmUfOfCfAmCm |
| 149 | UmUmCmGmCmUmUmCmA-p- |
| | (ps)2-GalNAc4 |
| | (SEQ ID NO: 56) |
| | d2vd3UpsfGpsmAmAfGmCmGfAm |
| | AmGmUmGmCfAmCmAfCmGmGps |
| | mUpsmC |
| | (SBQ ID NO: 155) |
| Control | mCpsmCpsmGmUfGmUfGfCfAm |
| ds-SINA- | CmUmUmCmGmCmUmUmCmA |
| 150 | (SEQ ID NO: 11) |
| | mUpsfGpsmAmAfGmCmGfAmAm |
| | GmUmGmCfAmCmAfCmGmGpsmUp |
| | smC |
| | (SEQ ID NO: 29) |

mX = 2′-O-methyl nucleotide;
fX = 2′-fluoro nucleotide;
dens\hat{ }G = 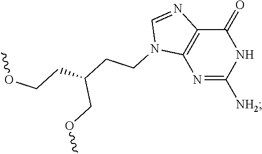
vmX = 5′-vinyl phosphonate 2′-O-methyl nucleotide;
mun 12G = 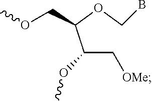
ps = phosphorothioate linkage;
X is a nucleobase (e.g. A, G, C, U or T).

Additionally or alternatively, the disclosed siNA may also incorporate further modifications to the nucleotide monomers. In some embodiments, the modifications include S′ TNA modifications such as coc-4 h on the antisense strand. Table 17 shows exemplary siNA comprising these modifications (bolded). In some embodiments, the siNA may comprise one or more of the disclosed modifications and the one or more disclosed modifications may be present in the sense strand or the antisense strand or both.

TABLE 17
siRNA with TNA phosphonate chemistry

| Name | SS/AS 5′ to 3′ |
|---|---|
| ds- | mCpsmCpsmGmUfGmUfGfCfAmCmUm |
| siNA- | UmCmGmCmUmUmCmA-(ps)2- |
| 151 | GalNAc4 |
| | (SEQ ID NO: 56) |
| | coc-4hpsfGpsmAmAfGmCmGfAmAm |
| | GmUmGmCfAmCmAfCmGmGpsm |
| | UpsmC |
| | (SEQ ID NO: 156) |

mX = 2′-O-methyl nucleotide;
fX = 2′-fluoro nucleotide;
coc-4h = 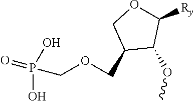
ps = phosphorothioate linkage;
X is a nucleobase (e.g. A, G, C, U or T).

Additionally or alternatively, the disclosed siNA may also incorporate further modifications to the nucleotide linkages. In some embodiments, the modifications include stereodefined PS linkages such as psr and pss on the antisense strand. Table 18 shows exemplary siNA comprising these modifications (bolded). In some embodiments, the siNA may comprise one or more of the disclosed modifications and the one or more disclosed modifications may be present in the sense strand or the antisense strand or both.

TABLE 18
siRNA Comprising Stereodefined PS linkages

| Name | SS/AS 5′ to 3′ |
|---|---|
| ds-siNA- | mCpsmCpsmGmUfGmUfGfCfAmCmUm |
| 156 | UmCmGmCmUmUmCmA-p-(ps)2-GalNAc4 |
| | (SEQ ID NO: 56) |
| | vmUpsr\hat{ }fGpsmAmAfGmCmGfAmAmGmUmGm |
| | CfAmCmAfCmGmGpsmUpsmC |
| | (SEQ ID NO: 161) |
| ds-siNA- | mCpsmCpsmGmUfGmUfGfCfAmCmUm |
| 157 | UmCmGmCmUmUmCmA-p-(ps)2-GalNAc4 |
| | (SEQ ID NO: 56) |
| | vmUpss\hat{ }fGpsmAmAfGmCmGfAmAmGm |
| | UmGmCfAmCmAfCmGmGpsmUpsmC |
| | (SEQ ID NO: 162) |
| ds-siNA- | mCpsmCpsmGmUfGmUfGfCfAmCmUm |
| 150 | UmCmGmCmUmUmCmA |
| | (SEQ ID NO: 11) |
| | mUpsfGpsmAmAfGmCmGfAmAmGmUmG |
| | mCfAmCmAfCmGmGpsmUpsmC |
| | (SEQ ID NO: 29) |
| ds-siNA- | mCpsmCpsmGmUfGmUfGfCfAmCmUmUm |
| 162 | CmGmCmUmUmCmA-p-(ps)2-GalNAc4 |
| | (SEQ ID NO: 56) |
| | mUpsfGpsmAmAfGmCmGfAmAmGm |
| | UmGmCfAmCmAfCmGmGpsmUpsmC |
| | (SEQ ID NO:29) |
| Control | fCpsmUpsfGmCfUmAfUmGfCmCfUmCfAmUfCmUfUmCfU |
| ds-siNA- | (SEQ ID NO: 165) |
| 158 | mApsfGpsmAfAmGfAmUfGmAfGmGfCmAf |
| | UmAfGmCfAmGpsmUpsmU |
| | (SEQ ID NO: 152) |

mX = 2′-O-methyl nucleotide;
fX = 2′-fluoro nucleotide;
pss?= 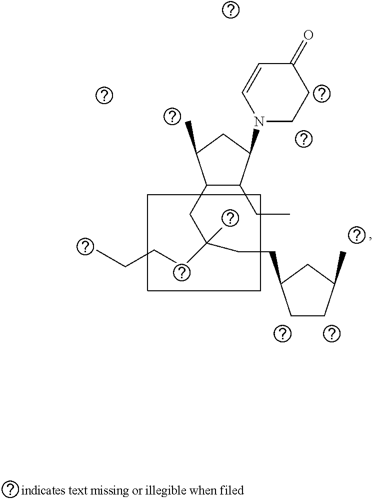 [SMILES: CCCCC(C)(CC[C@@H]1CC[C@H](C)C1)CC1C(CC)[C@H](N2C=CC(=O)CC2)C[C@@H]1C]
psr?= 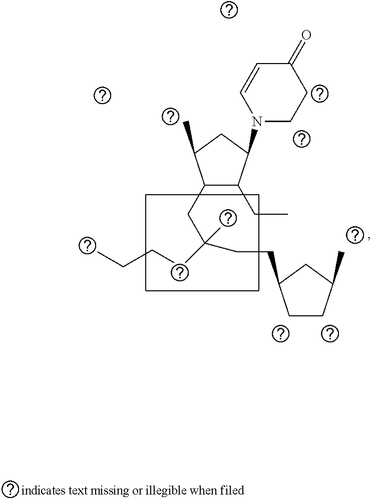 [SMILES: CCCCC(C)(CC[C@@H]1CC[C@H](C)C1)CC1C(CC)[C@H](N2C=CC(=O)CC2)C[C@@H]1C]
vmX = 5′-vinyl phosphonate 2′-O-methyl nucleotide;
ps = phosphorothioate linkage;
X is a nucleobase (e.g. A, G, C, U or T).

### Target Gene

Without wishing to be bound by theory, upon entry into a cell, any of the ds-siNA molecules disclosed herein may interact with proteins in the cell to form a RNA-Induced Silencing Complex (RISC). Once the ds-siNA is part of the RISC, the ds-siNA may be unwound to form a single-stranded siNA (ss-siNA). The ss-siNA may comprise the antisense strand of the ds-siNA. The antisense strand may bind to a complementary messenger RNA (mRNA), which results in silencing of the gene that encodes the mRNA.

The target gene may be any gene in a cell. In some embodiments, the target gene is a viral gene. In some embodiments, the viral gene is from a DNA virus. In some embodiments, the DNA virus is a double-stranded DNA (dsDNA) virus. In some embodiments, the dsDNA virus is a hepadnavirus. In some embodiments, the hepadnavirus is a hepatitis B virus (HBV). In some embodiments, the HBV is selected from HBV genotypes A-J. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+) ssRNA virus). In some embodiments, the (+) ssRNA virus is a coronavirus. In some embodiments, the coronavirus is a B-coronaviruses. In some embodiments, the B-coronaviruses is selected from the group consisting of severe acute respiratory syndrome coronavirus 2 (SARS-COV-2)(also known by the provisional name 2019 novel coronavirus, or 2019-nCOV), human coronavirus OC43 (hCoV-OC43), Middle East respiratory syndrome-related coronavirus (MERS-COV, also known by the provisional name 2012 novel coronavirus, or 2012-nCOV), and severe acute respiratory syndrome-related coronavirus (SARS-COV, also known as SARS-COV-1). In some embodiments, the B-coronaviruses is SARS-COV-2, the causative agent of COVID-19. Some exemplary target genes are shown in Table 23 at the end of the specification.

In some embodiments, the target gene is selected from the S gene or X gene of the HBV. In some embodiments, the HBV has a genome sequence shown in the nucleotide sequence of SEQ ID NO: 89 which corresponds to the nucleotide sequence of GenBank Accession No. U95551.1, which is incorporated by reference in its entirety.

An exemplary HBV genome sequence is shown in SEQ ID NO: 81, corresponding to Genbank Accession No. KC315400.1, which is incorporated by reference in its entirety. Nucleotides 2307 . . . 3215, 1 . . . 1623 of SEQ ID NO: 94 correspond to the polymerase/RT gene sequence, which encodes for the polymerase protein. Nucleotides 2848 . . . 3215, 1 . . . 835 of SEQ ID NO: 94 correspond to the PreS1/S2/S gene sequence, which encodes for the large S protein. Nucleotides 3205 . . . 3215, 1 . . . 835 of SEQ ID NO: 94 correspond to the PreS2/S gene sequence, which encodes for the middle S protein. Nucleotides 155 . . . 835 of SEQ ID NO: 94 correspond to the S gene sequence, which encodes the small S protein. Nucleotides 1374 . . . 1838 of SEQ ID NO: 94 correspond to the X gene sequence, which encodes the X protein. Nucleotides 1814 . . . 2452 of SEQ ID NO: 94 correspond to the PreC/C gene sequence, which encodes the precore/core protein. Nucleotides 1901 . . . 2452 of SEQ ID NO: 94 correspond to the C gene sequence, which encodes the core protein. The HBV genome further comprises viral regulatory elements, such as viral promoters (preS2, preS1, Core, and X) and enhancer elements (ENH1 and ENH2). Nucleotides 1624 . . . 1771 of SEQ ID NO: 94 correspond to ENH2. Nucleotides 1742 . . . 1849 of SEQ ID NO: 94 correspond to the Core promoter. Nucleotides 1818 . . . 3215, 1 . . . 1930 of SEQ ID NO: 94 correspond to the pregenomic RNA (pgRNA), which encodes the core and polymerase proteins.

In some embodiments, the sense strand comprises a sequence that is at least about 60%, 65%, 70%, 75%, 80%, 85%, 90%, 95%, or 100% complementary or hybridizes to a viral target RNA sequence that begins in an X region of HBV or in an S region of HBV. The viral target may, e.g., begin at the 5′-end of target-site in acc. KC315400.1 (genotype B, “gt B”), or in any one of genotypes A, C, or D. The skilled person would understand the HBV position, e.g., as described in Wing-Kin Sung, et al., Nature Genetics 44:765 (2012). In some embodiments, the S region is defined as from the beginning of small S protein (in genotype B KC315400.1 isolate, position #155) to before beginning of X protein (in genotype B KC315400.1 isolate, position #1373). In some embodiments, the X region is defined as from the beginning X protein (in genotype B KC315400.1 isolate, position #1374) to end of DR2 site (in genotype B KC315400.1 isolate, position #1603).

In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-720 or 1100-1700 of SEQ ID NO: 89. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-280, 300-445, 460-510, 650-720, 1170-1220, 1250-1300, or 1550-1630 of SEQ ID NO: 89. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-230, 250-280, 300-330, 370-400, 405-445, 460-500, 670-700, 1180-1210, 1260-1295, 1520-1550, or 1570-1610 of SEQ ID NO: 89. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides starting at position 203, 206, 254, 305, 375, 409, 412, 415, 416, 419, 462, 466, 467, 674, 676, 1182, 1262, 1263, 1268, 1526, 1577, 1578, 1580, 1581, 1583, or 1584 of SEQ ID NO: 89.

In some embodiments, the first nucleotide sequence is at least about 60%, 65%, 70%, 75%, 80%, 85%, 90%, 95%, or 100% identical to a nucleotide region within SEQ ID NO: 89, with the exception that the thymines (Ts) in SEQ ID NO: 89 are replaced with uracil (U). In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-720 or 1100-1700 of SEQ ID NO: 89. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-280, 300-445, 460-510, 650-720, 1170-1220, 1250-1300, or 1550-1630 of SEQ ID NO: 89. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-230, 250-280, 300-330, 370-400, 405-445, 460-500, 670-700, 1180-1210, 1260-1295, 1520-1550, or 1570-1610 of SEQ ID NO: 89. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides starting at position 203, 206, 254, 305, 375, 409, 412, 415, 416, 419, 462, 466, 467, 674, 676, 1182, 1262, 1263, 1268, 1526, 1577, 1578, 1580, 1581, 1583, or 1584 of SEQ ID NO: 89.

Several disease-causing coronaviruses share a high degree of homology in the regions of the genome encoding non-structural proteins (nsp), and more specifically, in the region encoding nsp8-nsp15. Indeed, there is roughly 65% identity across the roughly 7 kB sequence of β-coronaviruses from about nucleotide 12900 to about nucleotide 19900 of 2019-nCOV, and some subsections of the genomic span of nsp8 to nsp15 may comprise 95% or more identity. All of the genes in this region encode non-structural proteins associated with replication. Accordingly, this segment of the genome is suitable for targeting with an siNA that can provide a broad spectrum treatment for multiple different types of coronavirus, such as MERS-COV, SARS-COV-1, and SARS-COV-2.

In some embodiments, the target gene is selected from genome of SARS-COV-2. In some embodiments, SARS-COV-2 has a genome sequence shown in the nucleotide sequence of SEQ ID NO: 97, which corresponds to the nucleotide sequence of GenBank Accession No. NC_045512.2, which is incorporated by reference in its entirety. In some embodiments, the target gene a sequence 15 to 30, 15 to 25, 15 to 23, 17 to 23, 19 to 23, or 19 to 21 nucleotides in length, and preferably 19 or 21 nucleotides in length, within SEQ ID NO: 97. In some embodiments, the antisense strand sequence is complementary to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 97. In some embodiments, the sense strand sequence is identical to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 97.

In some embodiments, the target gene is selected from genome of SARS-COV. In some embodiments, SARS-COV has a genome corresponding to the nucleotide sequence of GenBank Accession No. NC_004718.3, which is incorporated by reference in its entirety.

In some embodiments, the target gene is selected from the genome of MERS-CoV. In some embodiments, MERS-COV has a genome corresponding to the nucleotide sequence of GenBank Accession No. NC_019843.3, which is incorporated by reference in its entirety.

In some embodiments, the target gene is selected from the genome of hCoV-OC43. In some embodiments, hCoV-OC43 has a genome corresponding to the nucleotide sequence of GenBank Accession No. NC_006213.1, which is incorporated by reference in its entirety.

In some embodiments, the target gene is involved in liver metabolism. In some embodiments, the target gene is an inhibitor of the electron transport chain. In some embodiments, the target gene encodes the MCJ protein (MCJ/DnaJC15 or Methylation-Controlled J protein). In some embodiments, the MCJ protein is encoded by the mRNA sequence of SEQ ID NO: 90, which corresponds to the nucleotide sequence of GenBank Accession No. NM_013238.3, which is incorporated by reference in its entirety.

In some embodiments, the target gene is TAZ. In some embodiments, TAZ comprises the nucleotide sequence of SEQ ID NO: 91, which corresponds to the nucleotide sequence of GenBank Accession No. NM_000116.5, which is incorporated by reference in its entirety.

In some embodiments, the target gene is angiopoietin like 3 (ANGPTL3). In some embodiments, ANGPTL3 comprises the nucleotide sequence of SEQ ID NO: 92, which corresponds to the nucleotide sequence of GenBank Accession No. NM_014495.4, which is incorporated by reference in its entirety. In some embodiments, the target gene is diacylglycerol acyltransferase 2 (DGAT2). In some embodiments, DGAT2 comprises the nucleotide sequence of SEQ ID NO: 93, which corresponds to the nucleotide sequence of GenBank Accession No. NM_001253891.1, which is incorporated by reference in its entirety.

### Compositions

As indicated above, the present disclosure provides compositions comprising any of the oligonucleotides, siNA molecules, sense strands, antisense strands, first nucleotide sequences, or second nucleotide sequences described herein. The compositions may comprise 1, 2, 3, 4, 5, 6, 7, 8, 9, 10, 11, 12 or more siNA molecules described herein. The compositions may comprise a first nucleotide sequence comprising a nucleotide sequence of any one of SEQ ID NOs: 1, 11, 28, 30-56, 69, 73, 98-103, 106, 158-160 and 165. In some embodiments, the composition comprises a second nucleotide sequence comprising a nucleotide sequence of any one of SEQ ID NOs: 2-10, 12-27, 29, 57-68, 70-72, 74-87, 104-157, and 161-164. In some embodiments, the composition comprises a sense strand comprising a nucleotide sequence of any one of SEQ ID NOs: 1, 11, 28, 30-56, 69, 73, 98-103, 106, 158-160 and 165. In some embodiments, the composition comprises an antisense strand comprising a nucleotide sequence of any one of SEQ ID NOs: 2-10, 12-27, 29, 57-68, 70-72, 74-87, 104-157, and 161-164.

Alternatively, the compositions may comprise (a) a phosphorylation blocker; and (b) a short interfering nucleic acid (siNA). In some embodiments, the phosphorylation blocker is any of the phosphorylation blockers disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide, a 2′-O-methyl nucleotide, a 2′-ocp nucleotide, a 2′-omcp nucleotide, a 3′-ocp nucleotide, a 3′-omcp nucleotide, a 2′-OMe-3′-Xylo nucleotide, a 2′-F-3′-xylo nucleotide, a Ganciclovir nucleotide, a Denvir nucleotide, a 5′-vinyl phosphonate 2′-O-methyl nucleotide, and a nucleotide analog. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) a conjugated moiety; and

(b) a short interfering nucleic acid (siNA). In some embodiments, the conjugated moiety is any of the galactosamines disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide, a 2′-O-methyl nucleotide, a 2′-ocp nucleotide, a 2′-omcp nucleotide, a 3′-ocp nucleotide, a 3′-omcp nucleotide, a 2′-OMe-3′-Xylo nucleotide, a 2′-F-3′-xylo nucleotide, a Ganciclovir nucleotide, a Denvir nucleotide, a 5′-vinyl phosphonate 2′-O-methyl nucleotide, and a nucleotide analog. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) a 5′-stabilized end cap; and (b) a short interfering nucleic acid (siNA). In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide, a 2′-O-methyl nucleotide, a 2′-ocp nucleotide, a 2′-omcp nucleotide, a 3′-ocp nucleotide, a 3′-omcp nucleotide, a 2′-OMe-3′-Xylo nucleotide, a 2′-F-3′-xylo nucleotide, a Ganciclovir nucleotide, a Denvir nucleotide, a 5′-vinyl phosphonate 2′-O-methyl nucleotide, and a nucleotide analog. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) at least one phosphorylation blocker, conjugated moiety, or 5′-stabilized end cap; and (b) a short interfering nucleic acid (siNA). In some embodiments, the phosphorylation blocker is any of the phosphorylation blockers disclosed herein. In some embodiments, the conjugated moiety is any of the galactosamines disclosed herein. In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide, a 2′-O-methyl nucleotide, a 2′-ocp nucleotide, a 2′-omcp nucleotide, a 3′-ocp nucleotide, a 3′-omcp nucleotide, a 2′-OMe-3′-Xylo nucleotide, a 2′-F-3′-xylo nucleotide, a Ganciclovir nucleotide, a Denvir nucleotide, a 5′-vinyl phosphonate 2′-O-methyl nucleotide, and the nucleotide analog. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

The composition may be a pharmaceutical composition. In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a siNA of the present disclosure which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., siNA molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siNA molecule) of the present disclosure. In certain embodiments, an aforementioned formulation renders orally bioavailable a compound (e.g., siNA molecule) of the present disclosure.

Methods of preparing these formulations or compositions include the step of bringing into association a compound (e.g., siNA molecule) of the present disclosure with the carrier and, optionally, one or more accessory ingredients. In general, the formulations are prepared by uniformly and intimately bringing into association a compound (e.g., siNA molecule) of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound (e.g., siNA molecule) of the present disclosure as an active ingredient. A compound (e.g., siNA molecule) of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds (e.g., siNA molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds (e.g., siNA molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., siNA molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., siNA molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., siNA molecule) of this disclosure include powders, sprays, ointments, pastes, creams, lotions, gels, solutions, patches and inhalants. The active compound (e.g., siNA molecule) may be mixed under sterile conditions with a pharmaceutically acceptable carrier, and with any preservatives, buffers, or propellants which may be required.

The ointments, pastes, creams and gels may contain, in addition to an active compound (e.g., siNA molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., siNA molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., siNA molecule) of the present disclosure to the body. Such dosage forms can be made by dissolving or dispersing the compound (e.g., siNA molecule) in the proper medium. Absorption enhancers can also be used to increase the flux of the compound (e.g., siNA molecule) across the skin. The rate of such flux can be controlled by either providing a rate controlling membrane or dispersing the compound (e.g., siNA molecule) in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds (e.g., siNA molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., siNA molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., siNA molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### Treatments and Administration

The siNA molecules of the present disclosure may be used to treat a disease in a subject in need thereof. In some embodiments, a method of treating a disease in a subject in need thereof comprises administering to the subject any of the siNA molecules disclosed herein. In some embodiments, a method of treating a disease in a subject in need thereof comprises administering to the subject any of the compositions disclosed herein.

The preparations (e.g., siNA molecules or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNA molecules) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNA molecules) of the disclosure employed in the pharmaceutical composition at levels lower than that required in order to achieve the desired therapeutic effect and gradually increase the dosage until the desired effect is achieved.

In general, a suitable daily dose of a compound (e.g., siNA molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

When the compounds (e.g., siNA molecules) described herein are co-administered with another, the effective amount may be less than when the compound is used alone.

If desired, the effective daily dose of the active compound (e.g., siNA molecule) may be administered as two, three, four, five, six or more sub-doses administered separately at appropriate intervals throughout the day, optionally, in unit dosage forms. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered once every 1, 2, 3, 4, 5, 6, 7, or 8 weeks.

### Diseases

The siNA molecules and compositions described herein may be administered to a subject to treat a disease. Further disclosed herein are uses of any of the siNA molecules or compositions disclosed herein in the manufacture of a medicament for treating a disease.

In some embodiments, the disease is a viral disease. In some embodiments, the viral disease is caused by a DNA virus. In some embodiments, the DNA virus is a double stranded DNA (dsDNA virus). In some embodiments, the dsDNA virus is a hepadnavirus. In some embodiments, the hepadnavirus is a hepatitis B virus (HBV).

In some embodiments, the disease is a liver disease. In some embodiments, the liver disease is nonalcoholic fatty liver disease (NAFLD). In some embodiments, the NAFLD is nonalcoholic steatohepatitis (NASH). In some embodiments, the liver disease is hepatocellular carcinoma (HCC).

The siNA molecules of the present disclosure may be used to treat or prevent a disease in a subject in need thereof. In some embodiments, a method of treating or preventing a disease in a subject in need thereof comprises administering to the subject any of the siNA molecules disclosed herein. In some embodiments, a method of treating or preventing a disease in a subject in need thereof comprises administering to the subject any of the compositions disclosed herein.

In some embodiments of the disclosed methods and uses, the disease is a respiratory disease. In some embodiments, the respiratory disease is a viral infection. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (e.g., COVID-19). In some embodiments, the respiratory disease can include one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can include complications selected from sinusitis, otitis media, pneumonia, acute respiratory distress syndrome, disseminated intravascular coagulation, pericarditis, and kidney failure. In some embodiments, the respiratory disease is idiopathic.

In some embodiments, the present disclosure provides methods of treating or preventing a coronavirus infection, comprising administering to a subject in need thereof a therapeutically effective amount of one or more of the siNAs or a pharmaceutical composition as disclosed herein. In some embodiments, the coronavirus infection is selected from the group consisting of Middle East Respiratory Syndrome (MERS), Severe Acute Respiratory Syndrome (SARS), and COVID-19. In some embodiments, the subject has been treated with one or more additional coronavirus treatment agents. In some embodiments, the subject is concurrently treated with one or more additional coronavirus treatment agents.

Administration of siNA

Administration of any of the siNAs disclosed herein may be conducted by methods known in the art. In some embodiments, the siNA is administered by subcutaneous (SC) or intravenous (IV) delivery. The preparations (e.g., siNAs or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. In some embodiments, subcutaneous administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNAs) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNAs) of the disclosure employed in the pharmaceutical composition at levels lower than that required in order to achieve the desired therapeutic effect and gradually increase the dosage until the desired effect is achieved.

In general, a suitable daily dose of a compound (e.g., siNA) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at about 1 mg/kg to about 40 mg/kg, about 1 mg/kg to about 30 mg/kg, about 1 mg/kg to about 20 mg/kg, about 1 mg/kg to about 15 mg/kg, or 1 mg/kg to about 10 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

If desired, the effective daily dose of the active compound (e.g., siNA) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, any one of the siNAs or compositions disclosed herein is administered in a particle or viral vector. In some embodiments, the viral vector is a vector of adenovirus, adeno-associated virus (AAV), alphavirus, flavivirus, herpes simplex virus, lentivirus, measles virus, picornavirus, poxvirus, retrovirus, or rhabdovirus. In some embodiments, the viral vector is a recombinant viral vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

The subject of the described methods may be a mammal, and it includes humans and non-human mammals. In some embodiments, the subject is a human, such as an adult human.

Some embodiments include a method for treating an HBV virus in a subject infected with the virus comprising administering a therapeutically effective amount of one or more siNA of the present disclosure or a composition of the present disclosure to the subject in need thereof thereby reducing the viral load of the virus in the subject and/or reducing a level of a virus antigen in the subject. The siNA may be complementary or hybridize to a portion of the target RNA in the virus, e.g., an X region and/or an S region of HBV.

### Combination Therapies

Any of the methods disclosed herein may further comprise administering to the subject an additional HBV treatment agent. Any of the compositions disclosed herein may further comprise an additional HBV treatment agent. In some embodiments, the additional HBV treatment agent is selected from a nucleotide analog, nucleoside analog, a capsid assembly modulator (CAM), a recombinant interferon, an entry inhibitor, a small molecule immunomodulator and oligonucleotide therapy. In some embodiments, the additional HBV treatment agent is selected from HBV STOPS™ ALG-010133, HBV CAM ALG-000184, ASO 1 (SEQ ID NO: 95), ASO 2 (SEQ ID NO: 96) recombinant interferon alpha 2b, IFN-α, PEG-IFN-a-2a, lamivudine, telbivudine, adefovir dipivoxil, clevudine, entecavir, tenofovir alafenamide, tenofovir disoproxil, NVR3-778, BAY41-4109, JNJ=632, JNJ=3989 (ARO-HBV), RG6004, GSK3228836, REP-2139, REP-2165, AB-729, VIR-2218, RG6346 (DCR-HBVS), JNJ=6379, GLS4, ABI-HO731, JNJ=440, NZ-4, RG7907, EDP-514, AB-423, AB-506, ABI-H03733 and ABI-H2158. In some embodiments, the oligonucleotide therapy is selected from Nucleic Acid Polymers or S-Antigen Transport-inhibiting Oligonucleotide Polymers (NAPs or STOPS), siRNA, and ASO. In some embodiments, the oligonucleotide therapy is an additional siNA. In some embodiments, the additional siNA is selected from any of ds-siNA-001 to ds-siNA-092. In some embodiments, the oligonucleotide therapy is an antisense oligonucleotide (ASO). In some embodiments, the ASO is ASO 1 (SEQ ID NO: 95) or ASO 2 (SEQ ID NO: 96). In some embodiments, any of the siNAs disclosed herein are co-administered with STOPS. Exemplary STOPS are described in International Publication No. WO2020/097342 and U.S. Publication No. 2020/0147124, both of which are incorporated by reference in their entirety. In some embodiments, the STOPS is ALG-010133. In some embodiments, any of the siNAs disclosed herein are co-administered with tenofovir. In some embodiments, any of the siNAs disclosed herein are co-administered with a CAM. Exemplary CAMs are described in Berke et al., Antimicrob Agents Chemother, 2017, 61 (8): e00560-17, Klumpp, et al., Gastroenterology, 2018, 154 (3): 652-662.e8, International Application Nos. PCT/US2020/017974, PCT/US2020/026116, and PCT/US2020/028349 and U.S. application Ser. Nos. 16/789,298, 16/837,515, and 16/849,851, each which is incorporated by reference in its entirety. In some embodiments, the CAM is ALG-000184, ALG-001075, ALG-001024, JNJ=632, BAY41-4109, or NVR3-778. In some embodiments, the siNA and the HBV treatment agent are administered simultaneously. In some embodiments, the siNA and the HBV treatment agent are administered concurrently. In some embodiments, the siNA and the HBV treatment agent are administered sequentially. In some embodiments, the siNA is administered prior to administering the HBV treatment agent. In some embodiments, the siNA is administered after administering the HBV treatment agent. In some embodiments, the siNA and the HBV treatment agent are in separate containers. In some embodiments, the siNA and the HBV treatment agent are in the same container.

Any of the methods disclosed herein may further comprise administering to the subject a liver disease treatment agent. Any of the compositions disclosed herein may further comprise a liver disease treatment agent. In some embodiments, the liver disease treatment agent is selected from a peroxisome proliferator-activator receptor (PPAR) agonist, farnesoid X receptor (FXR) agonist, lipid-altering agent, and incretin-based therapy. In some embodiments, the PPAR agonist is selected from a PPARα agonist, dual PPARα/δ agonist, PPARγ agonist, and dual PPARα/γ agonist. In some embodiments, the dual PPARα agonist is a fibrate. In some embodiments, the PPARα/δ agonist is elafibranor. In some embodiments, the PPARγ agonist is a thiazolidinedione (TZD). In some embodiments, TZD is pioglitazone. In some embodiments, the dual PPARα/γ agonist is saroglitazar. In some embodiments, the FXR agonist is obeticholic acis (OCA). In some embodiments, the lipid-altering agent is aramchol. In some embodiments, the incretin-based therapy is a glucagon-like peptide 1 (GLP-1) receptor agonist or dipeptidyl peptidase 4 (DPP-4) inhibitor. In some embodiments, the GLP-1 receptor agonist is exenatide or liraglutide. In some embodiments, the DPP-4 inhibitor is sitagliptin or vildapliptin. In some embodiments, the siNA and the liver disease treatment agent are administered concurrently. In some embodiments, the siNA and the liver disease treatment agent are administered sequentially. In some embodiments, the siNA is administered prior to administering the liver disease treatment agent. In some embodiments, the siNA is administered after administering the liver disease treatment agent. In some embodiments, the siNA and the liver disease treatment agent are in separate containers. In some embodiments, the siNA and the liver disease treatment agent are in the same container.

### Phosphoamidites

In addition to the disclosed oligonucleotides comprising novel nucleotide monomers, the present disclosure also provides phosphoramidite selected from:

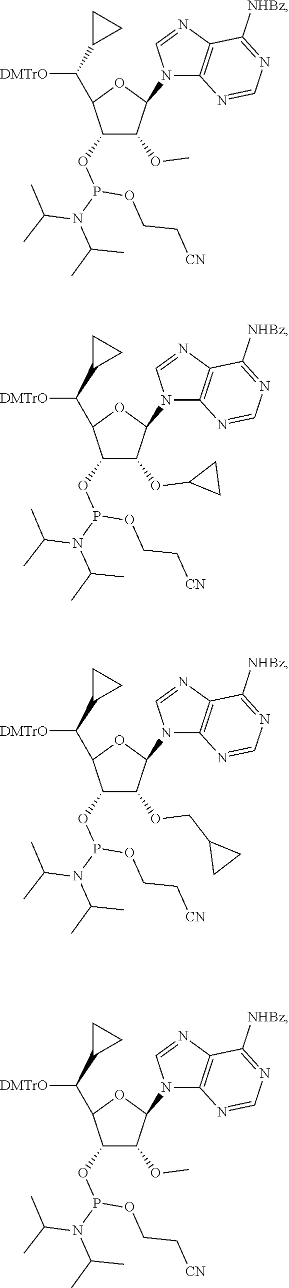

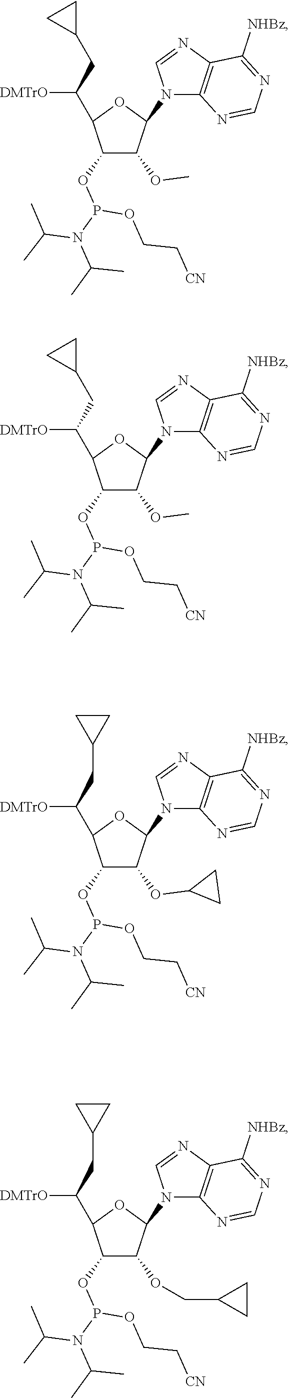

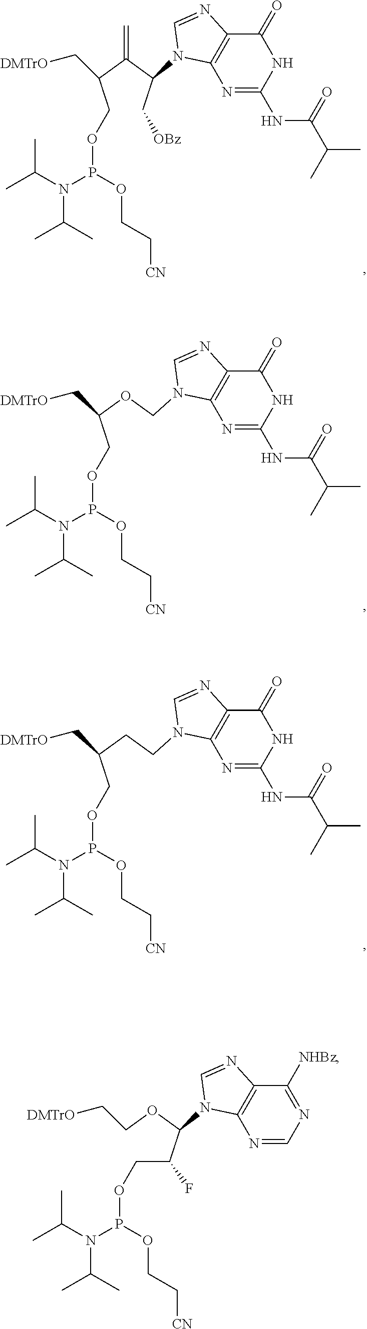

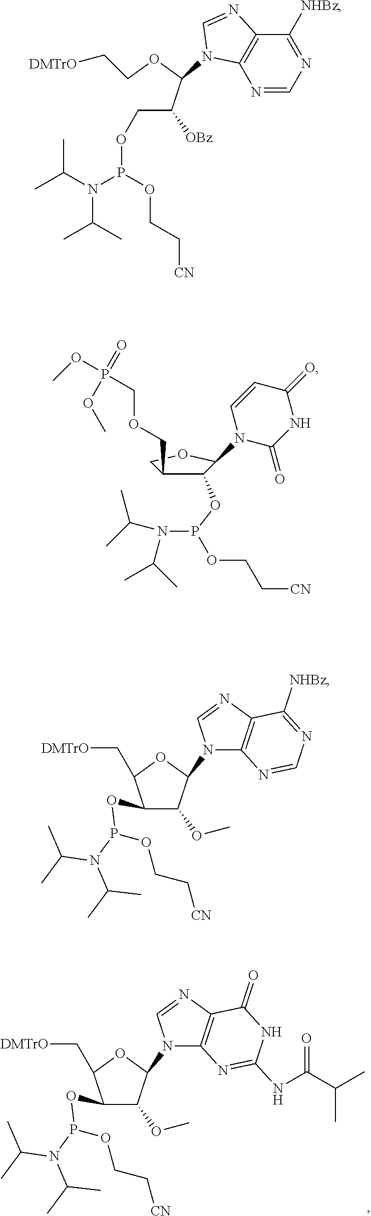

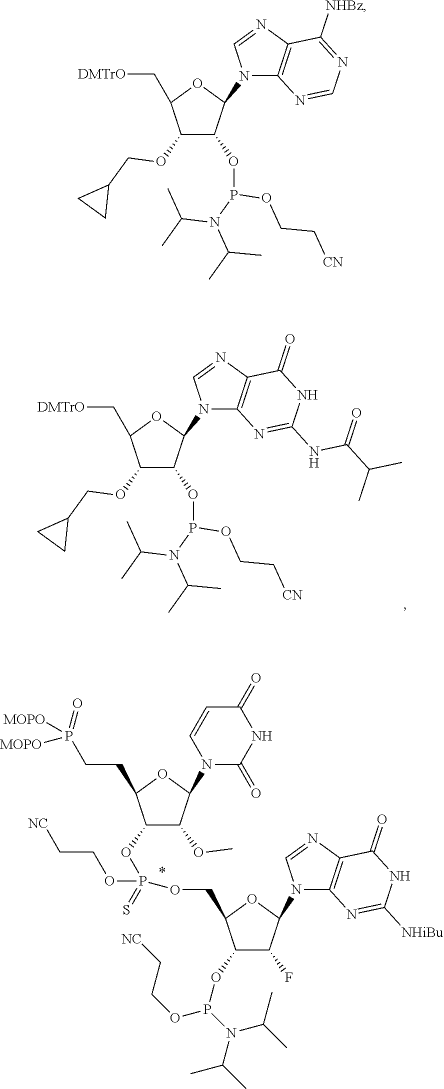

wherein * is a chiral center,

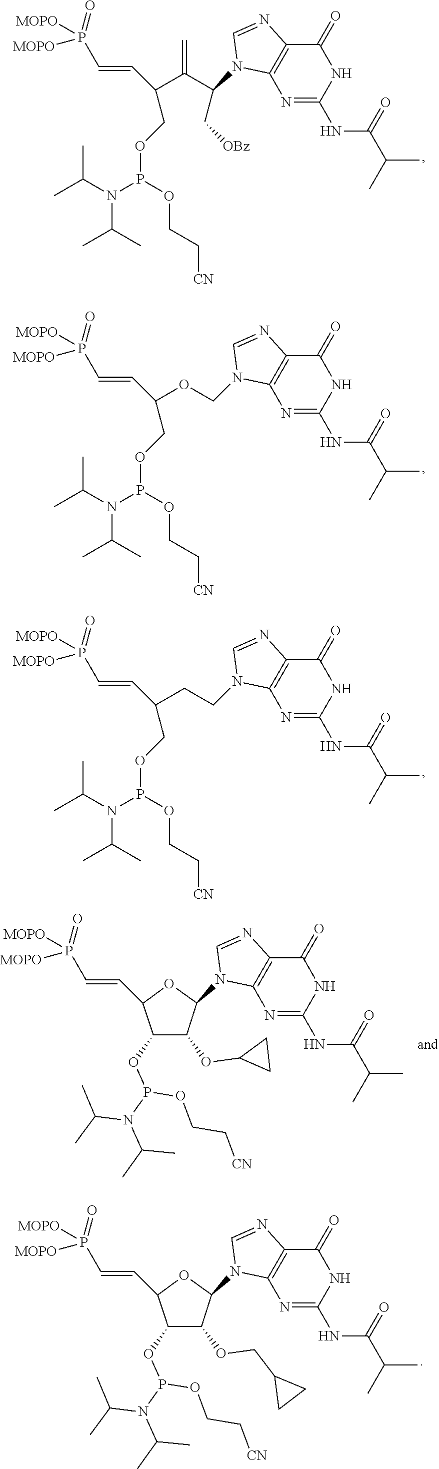

Those skilled in the art will understand that the disclosed phosphoramidites may be used in the synthesis of the disclosed nucleotide monomers.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

The terms “a” and “an” as used herein mean “one or more” and include the plural unless the context is inappropriate.

As used herein, the terms “patient” and “subject” refer to organisms to be treated by the methods of the present disclosure. Such organisms are preferably mammals (e.g., marines, simians, equines, bovines, porcinis, canines, felines, and the like), and more preferably humans.

As used herein, the term “effective amount” refers to the amount of a compound (e.g., a siNA of the present disclosure) sufficient to effect beneficial or desired results. An effective amount can be administered in one or more administrations, applications, or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the terms “alleviate” and “alleviating” refer to reducing the severity of the condition, such as reducing the severity by, for example, at least 10%, 20%, 30%, 40%, 50%, 60%, 70%, 80%, 90%, or 95%.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

The term “about” as used herein when referring to a measurable value (e.g., weight, time, and dose) is meant to encompass variations, such as +10%, +5%, =1%, or +0.1% of the specified value.

As used herein, the term “nucleobase” refers to a nitrogen-containing biological compound that forms a nucleoside. Examples of nucleobases include, but are not limited to, thymine, uracil, adenine, cytosine, guanine, and an analog or derivative thereof.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates that may need to be independently confirmed.

### EXAMPLES

### Example 1: siNA Synthesis

This example describes an exemplary method for synthesizing ds-siNAs, such as the siNAs disclosed in Tables 1-15 (as identified by the ds-siNA ID).

The 2′-OMe phosphoramidite 5′-O-DMT-deoxy Adenosine (NH-Bz), 3′-O-(2-cyanoethyl-N,N-diisopropyl phosphoramidite, 5′-O-DMT-deoxy Guanosine (NH-ibu), 3′-O-(2-cyanoethyl-N,N-diisopropyl phosphoramidite, 5′-O-DMT-deoxy Cytosine (NH-Bz), 3′-O-(2-cyanoethyl-N,N-diisopropyl phosphoramidite, 5′-O-DMT-Uridine 3′-O-(2-cyanoethyl-N,N-diisopropyl phosphoramidite and solid supports were purchased from Chemgenes Corp. MA.

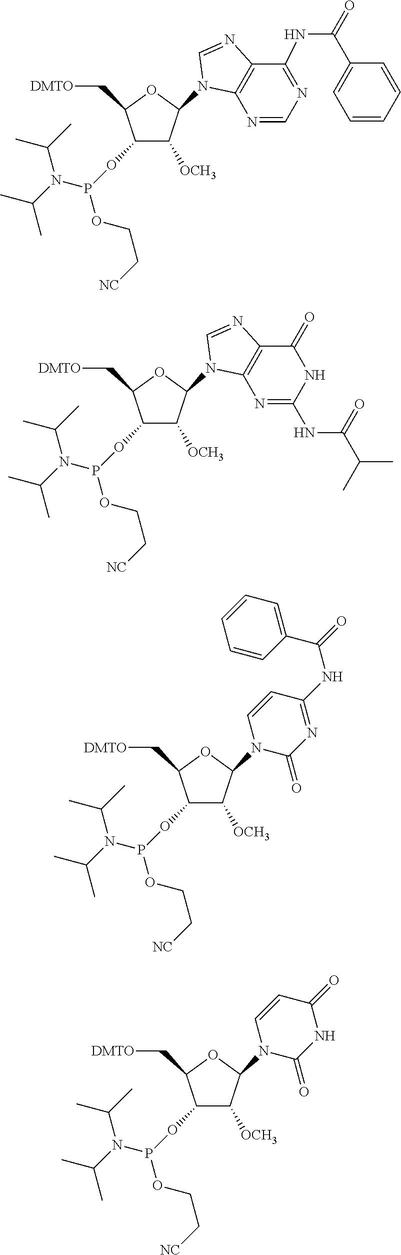

The 2′-F-5′-O-DMT-(NH-Bz) Adenosine-3′-O-(2-cyanoethyl-N,N-diisopropyl phosphoramidite, 2′-F-5′-O-DMT-(NH-ibu)-Guanosine, 3′-O-(2-cyanoethyl-N,N-diisopropyl phosphoramidite, 5′-O-DMT-(NH-Bz)-Cytosine, 2′-F-3′-O-(2-cyanoethyl-N,N-diisopropyl phosphoramidite, 5′-O-DMT-Uridine, 2′-F-3′-O-(2-cyanoethyl-N,N-diisopropyl phosphoramidite and solid supports were purchased from Thermo Fischer Milwaukee WI, USA.

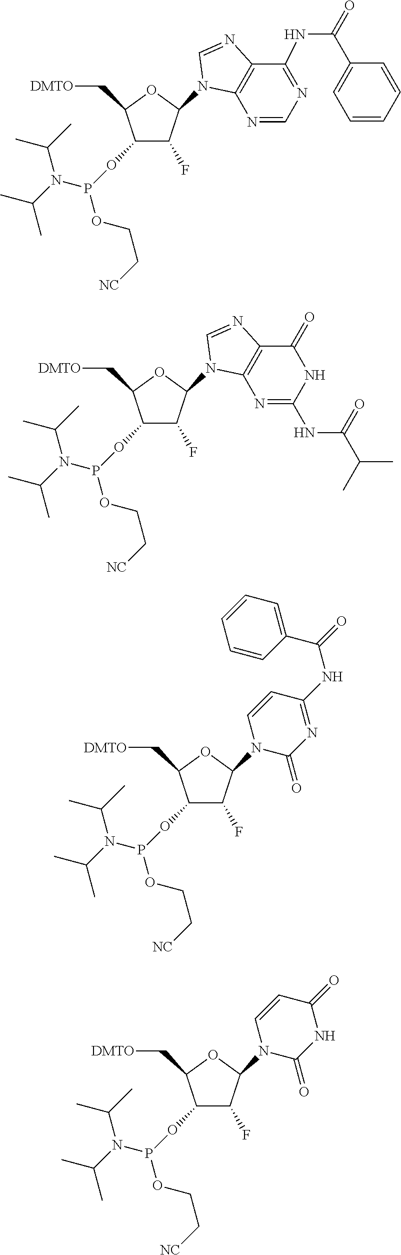

All the monomers were dried in vacuum desiccator with desiccants (P₂O₅, RT 24 h). The solid supports (CPG) attached to the nucleosides and universal supports was obtained from LGC and Chemgenes. The chemicals and solvents for post synthesis workflow were purchased from commercially available sources like VWR/Sigma and used without any purification or treatment. Solvent (Acetonitrile) and solutions (amidite and activator) were stored over molecular sieves during synthesis.

The oligonucleotides were synthesized on a DNA/RNA Synthesizers (Expedite 8909 or ABI-394 or MM-48) using standard oligonucleotide phosphoramidite chemistry starting from the 3′ residue of the oligonucleotide preloaded on CPG support. An extended coupling of 0.1M solution of phosphoramidite in CH₃CN in the presence of 5-(ethylthio)-1H-tetrazole activator to a solid bound oligonucleotide followed by standard capping, oxidation and deprotection afforded modified oligonucleotides. The 0.1M I₂, THF:Pyridine; Water-7:2:1 was used as oxidizing agent while DDTT ((dimethylamino-methylidene)amino)-3H-1,2,4-dithiazoline-3-thione was used as the sulfur-transfer agent for the synthesis of oligoribonucleotide phosphorothioates. The stepwise coupling efficiency of all modified phosphoramidites was more than 98%.

| Reagents | Detailed Description |
|---|---|
| Deblock Solution | 3% Dichloroacetic acid (DCA) in Dichloromethane |
| Amidite | 0.1M in Anhydrous Acetonitrile |
| Concentration | |
| Activator | 0.25M Ethyl-thio-Tetrazole (ETT) |
| Cap-A solution | Acetic anhydride in pyridine/THF |
| Cap-B Solution | 16% 1-Methylimidazole in THF |
| Oxidizing Solution | 0.02M I₂, THF:pyridine; water-7:2:1 |
| Sulfurizing Solution | 0.2M DDTT in pyridine/acetonitrile 1:1 |

### Cleavage and Deprotection:

Deprotection and cleavage from the solid support was achieved with mixture of ammonia methylamine (1:1, AMA) for 15 min at 65° C. When the universal linker was used, the deprotection was left for 90 min at 65° C. or solid supports were heated with aqueous ammonia (28%) solution at 55° C. for 8-16 h to deprotect the base labile protecting groups.

Quantitation of Crude siNA or Raw Analysis

Samples were dissolved in deionized water (1.0 mL) and quantitated as follows: Blanking was first performed with water alone (2 μl) on Thermo Scientific™ Nanodrop UV spectrophotometer or BioTek™ Epoch™ plate reader then Oligo sample reading was obtained at 260 nm. The crude material is dried down and stored at −20° C.

### Crude HPLC/LC-MS Analysis

The 0.1 OD of the crude samples were analyzed for crude HPLC and LC-MS analysis. After Confirming the crude LC-MS data then purification step was performed if needed based on the purity.

### HPLC Purification

The unconjugated and GalNac modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.0 M NaBr, pH 8.5 (buffer B). Fractions containing full-length oligonucleotides were pooled.

### Desalting of Purified SiNA

The purified dry siNA was then desalted using Sephadex G-25 M (Amersham Biosciences). The cartridge was conditioned with 10 mL of deionized water thrice. Finally, the purified siNA dissolved thoroughly in 2.5 mL RNAse free water was applied to the cartridge with very slow drop wise elution. The salt free siNA was eluted with 3.5 ml deionized water directly into a screw cap vial. Alternatively, some unconjugated siNA was deslated using Pall AcroPrep™ 3K MWCO desalting plates.

### IEX HPLC and Electrospray LC/MS Analysis

Approximately 0.10 OD of siNA is dissolved in water and then pipetted into HPLC autosampler vials for IEX-HPLC and LC/MS analysis. Analytical HPLC and ES LC-MS confirmed the identity and purity of the compounds.

### Duplex Preparation:

Single strand oligonucleotides (Sense and Antisense strands) were annealed (1:1 by molar equivalents, heat at 90° C. for 2 min followed by gradual cooling at room temperature) to give the duplex ds-siNA. The final compounds were analyzed on size exclusion chromatography (SEC).

### Example 2: ds-siNA Activity

This example investigates the activity of the ds-siNAs synthesized in Example 1.

Homo sapiens HepG2.2.15 cells were cultured in Dulbecco's Modified Eagle's Medium (DMEM) (ATCC 30-2002) supplemented to also contain 10% fetal calf serum (FCS). Cells were incubated at 37° C. in an atmosphere with 5% CO₂ in a humidified incubator. For transfection of HepG2.2.15 cells with HBV targeting siRNAs, cells were seeded at a density of 15000 cells/well in 96-well regular tissue culture plates. Transfection of cells was carried out using RNAiMAX (Invitrogen/Life Technologies) according to the manufacturer's instructions. Dose-response experiments were done with oligo concentrations of 40, 20, 10, 5, 2.5, 1.25, 0.625, 0.3125, 0.15625 and 0.07813 nM. For each HBV targeting siRNA treatment, four wells were transfected in parallel, and individual data points were collected from each well. After 24 h of incubation with siRNA, media was removed, and cells were lysed and analyzed with a QuantiGene2.0 branched DNA (bDNA) probe set specific for HBV genotype D (also called Hepatitis B virus subtype ayw, complete genome of 3182 base-pairs) as present in cell line HepG2.2.15.

For each well, the HBV on-target mRNA levels were normalized to the GAPDH mRNA level. As shown in Tables 9-17, the activity of the HBV targeting ds-siRNAs was expressed as EC50, 50% reduction of normalized HBV RNA level from no drug control. As shown in Tables 9-17, the cytotoxicity of the HBV targeting ds-siRNAs was expressed by CC50 of 50% reduction of GAPDH mRNA from no drug control.

### Example 3: Use of Ds-siNAs to Treat Hepatitis B Virus Infection

In this example, the ds-siNAs synthesized in Example 1 are used to treat a hepatitis B virus infection in a subject. Generally, a composition comprising a ds-siNA from Tables 1-8 (as identified by the ds-siNA ID) and a pharmaceutically acceptable carrier is administered to the subject suffering from hepatitis B virus. The ds-siNA from Tables 1-8 may be conjugated to N-acetylgalactosamine. The ds-siNA is administered at a dose of 0.3 to 5 mg/kg every three weeks by subcutaneous injection or intravenous infusion.

### Example 4: siNA Activity Assays

This example provides exemplary methods for testing the activity of the siNAs disclosed herein.

### In Vitro Assay:

HepG2.2.15 cells were cultured in Dulbecco's Modified Eagle's Medium (DMEM) (ATCC 30-2002) supplemented to also contain 10% fetal calf serum (FCS). Cells were incubated at 37° C. in an atmosphere with 5% CO₂ in a humidified incubator. For transfection of HepG2.2.15 cells with HBV targeting siRNAs, cells were seeded at a density of 15000 cells/well in 96-well regular tissue culture plates. Transfection of cells was carried out using RNAiMAX (Invitrogen/Life Technologies) according to the manufacturer's instructions. Dose-response experiments were done with oligo concentrations of 40, 20, 10, 5, 2.5, 1.25, 0.625, 0.3125, 0.15625 and 0.07813 nM. For each HBV targeting siRNA treatment (e.g., ds-siRNA, as identified by the ds-siNA ID in Tables 9-17), four wells were transfected in parallel, and individual data points were collected from each well. After 24 h of incubation with siRNA, media was removed, and cells were lysed and analyzed with a QuantiGene2.0 branched DNA (bDNA) probe set specific for HBV genotype D (also called Hepatitis B virus subtype ayw, complete genome of 3182 base-pairs) as present in cell line HepG2.2.15.

For each well, the HBV on-target mRNA levels were normalized to the GAPDH mRNA level. As shown in Tables 19-33 and 36-40, the activity of the HBV targeting ds-siRNAs was expressed as EC50, 50% reduction of normalized HBV RNA level from no drug control. As shown in Tables 19-33 and 36-40, the cytotoxicity of the HBV targeting ds-siRNAs was expressed by CC50 of 50% reduction of GAPDH mRNA from no drug control.

Off target results were also measured for certain siNAs. Table 34 shows IC50 of variants including ganciclovir and danovir nucleotides improved off target activity by greater than 1000× compared to the control. Table 35 shows IC50 of variants including G analogs and vinyl phosphate 5′ end caps also improved off target activity over the control.

TABLE 19
siNA Comprising N1-stabilizing Nucleotides

| | | | EC₅₀ | Emax | CC₅₀ |
|---|---|---|---|---|---|
| Name | Sense | Antisense | (nM) | (%) | (nM) |
| ds-siNA-001 | (SEQ ID NO: 1) | (SEQ ID NO: 2) | 0.037 | 83.44 | >1 |
| ds-siNA-002 | (SEQ ID NO: 1) | (SEQ ID NO: 3) | 0.013 | 77.11 | >1 |
| ds-siNA-003 | (SEQ ID NO: 1) | (SEQ ID NO: 4) | 0.026 | 67.71 | >1 |
| ds-siNA-004 | (SEQ ID NO: 1) | (SEQ ID NO: 5) | 0.035 | 74.99 | >1 |
| ds-siNA-005 | (SEQ ID NO: 1) | (SEQ ID NO: 6) | 0.043 | 66.52 | >1 |
| ds-siNA-006 | (SEQ ID NO: 1) | (SEQ ID NO: 7) | 0.035 | 74.11 | >1 |
| ds-siNA-007 | (SEQ ID NO: 1) | (SEQ ID NO: 8) | 0.032 | 71.84 | >1 |
| ds-siNA-008 | (SEQ ID NO: 1) | (SEQ ID NO: 9) | 0.033 | 68.17 | >1 |
| ds-siNA-009 | (SEQ ID NO: 1) | (SEQ ID NO: 10) | 0.019 | 87.62 | >1 |

TABLE 20
siNA Comprising 2′-ocp and 2′-omcp Nucleotides - 1

| | | | EC₅₀ | Emax | CC₅₀ |
|---|---|---|---|---|---|
| Name | Sense | Antisense | (pM) | (%) | (pM) |
| ds-siNA-010 | (SEQ ID NO: 11) | (SEQ ID NO: 12) | 10 | 80.94 | >1000 |
| ds-siNA-011 | (SEQ ID NO: 11) | (SEQ ID NO: 13) | 6 | 79.19 | >1000 |
| ds-siNA-012 | (SEQ ID NO: 11) | (SEQ ID NO: 14) | 6 | 77.27 | >1000 |
| ds-siNA-013 | (SEQ ID NO: 11) | (SEQ ID NO: 15) | 10 | 77.38 | >1000 |
| ds-siNA-014 | (SEQ ID NO: 11) | (SEQ ID NO: 16) | 6 | 71.22 | >1000 |
| ds-siNA-015 | (SEQ ID NO: 11) | (SEQ ID NO: 17) | 7 | 77.97 | >1000 |
| ds-siNA-016 | (SEQ ID NO: 11) | (SEQ ID NO: 18) | 647 | 100.0 | >1000 |
| ds-siNA-017 | (SEQ ID NO: 11) | (SEQ ID NO: 19) | 169 | 95.26 | >1000 |
| ds-siNA-019 | (SEQ ID NO: 11) | (SEQ ID NO: 20) | 4 | 71.88 | >1000 |
| ds-siNA-020 | (SEQ ID NO: 11) | (SEQ ID NO: 21) | 11 | 78.94 | >1000 |
| ds-siNA-021 | (SEQ ID NO: 11) | (SEQ ID NO: 22) | 24 | 75.29 | >1000 |
| ds-siNA-022 | (SEQ ID NO: 11) | (SEQ ID NO: 23) | 11 | 81.39 | >1000 |
| ds-siNA-023 | (SEQ ID NO: 11) | (SEQ ID NO: 24) | 12 | 78.41 | >1000 |
| Control | (SEQ ID NO: 11) | (SEQ ID NO: 29) | 10 | 72.61 | >1000 |
| ds-siNA-150 | | | | | |

TABLE 21
siNA Comprising 2′-ocp and 2′-omcp Nucleotides - 2

| | | | EC₅₀ | Emax | CC₅₀ |
|---|---|---|---|---|---|
| Name | Sense | Antisense | (pM) | (%) | (pM) |
| ds-siNA- | (SEQ ID NO: 11) | (SEQ ID NO: 25) | 35 | 88.21 | >1000 |
| 024 | | | | | |
| Control | (SEQ ID NO: 11) | (SEQ ID NO: 29) | 21 | 79.24 | >1000 |
| ds-siNA- | | | | | |
| 150 | | | | | |

TABLE 22
siNA Comprising 2′-ocp and 2′-omcp Nucleotides - 3

| | | | EC₅₀ | CC₅₀ |
|---|---|---|---|---|
| Name | Sense | Antisense | (nM) | (nM) |
| ds-siNA-025 | (SEQ ID NO: 11) | (SEQ ID NO: 26) | 0.0061 | >0.1 |
| ds-siNA-026 | (SEQ ID NO: 11) | (SEQ ID NO: 27) | 0.0053 | >0.1 |
| Control | (SEQ ID NO: 11) | (SEQ ID NO: 29) | 0.012 | >1 |
| ds-siNA-150 | | | | |

TABLE 23
siNA Comprising 2′-ocp and 2′-omcp Nucleotides and Conjugated Moieties

| | | | EC₅₀ | EMax | CC₅₀ |
|---|---|---|---|---|---|
| Name | Sense | Antisense | (nM) | (%) | (nM) |
| ds-siNA-027 | (SEQ ID NO: 28) | (SEQ ID NO: 29) | 11 | 82.54 | >1000 |
| ds-siNA-028 | (SEQ ID NO: 30) | (SEQ ID NO: 29) | 21 | 87.04 | >1000 |
| ds-siNA-029 | (SEQ ID NO: 31) | (SEQ ID NO: 29) | 12 | 79.1 | >1000 |
| ds-siNA-030 | (SEQ ID NO: 32) | (SEQ ID NO: 29) | 6 | 86.41 | >1000 |
| ds-siNA-031 | (SEQ ID NO: 33) | (SEQ ID NO: 29) | 6 | 81.48 | >1000 |
| ds-siNA-032 | (SEQ ID NO: 34) | (SEQ ID NO: 29) | 7 | 82.8 | >1000 |
| ds-siNA-033 | (SEQ ID NO: 35) | (SEQ ID NO: 29) | 10 | 85.29 | >1000 |
| ds-siNA-034 | (SEQ ID NO: 36) | (SEQ ID NO: 29) | 11 | 81.3 | >1000 |
| ds-siNA-035 | (SEQ ID NO: 37) | (SEQ ID NO: 29) | 9 | 84.73 | >1000 |
| ds-siNA-036 | (SEQ ID NO: 38) | (SEQ ID NO: 29) | 17 | 82.35 | >1000 |
| ds-siNA-037 | (SEQ ID NO: 39) | (SEQ ID NO: 29) | 10 | 81.98 | >1000 |
| ds-siNA-038 | (SEQ ID NO: 40) | (SEQ ID NO: 29) | 13 | 81.51 | >1000 |
| ds-siNA-039 | (SEQ ID NO: 41) | (SEQ ID NO: 29) | 6 | 78.38 | >1000 |
| ds-siNA-040 | (SEQ ID NO: 42) | (SEQ ID NO: 29) | 7 | 83.67 | >1000 |
| ds-siNA-041 | (SEQ ID NO: 43) | (SEQ ID NO: 29) | 12 | 80.08 | >1000 |
| ds-siNA-042 | (SEQ ID NO: 44) | (SEQ ID NO: 29) | 20 | 82.99 | >1000 |
| ds-siNA-043 | (SEQ ID NO: 45) | (SEQ ID NO: 29) | 50 | 91.31 | >1000 |
| ds-siNA-044 | (SEQ ID NO: 46) | (SEQ ID NO: 29) | 73 | 88.83 | >1000 |
| ds-siNA-045 | (SEQ ID NO: 47) | (SEQ ID NO: 29) | 14 | 84.74 | >1000 |
| ds-siNA-046 | (SEQ ID NO: 48) | (SEQ ID NO: 29) | 4 | 70.38 | >1000 |
| ds-siNA-047 | (SEQ ID NO: 49) | (SEQ ID NO: 29) | 19 | 80.16 | >1000 |
| ds-siNA-048 | (SEQ ID NO: 50) | (SEQ ID NO: 29) | 15 | 85.58 | >1000 |
| ds-siNA-049 | (SEQ ID NO: 51) | (SEQ ID NO: 29) | 12 | 81.65 | >1000 |
| ds-siNA-050 | (SEQ ID NO: 52) | (SEQ ID NO: 29) | 11 | 79.95 | >1000 |
| ds-siNA-051 | (SEQ ID NO: 53) | (SEQ ID NO: 29) | 12 | 84.56 | >1000 |
| Control | | | | | |

TABLE 24
siNA Comprising Alternating 2′-ocp or 2′-omcp Nucleotides

| | | | EC₅₀ | CC₅₀ |
|---|---|---|---|---|
| Name | Sense | Antisense | (nM) | (nM) |
| ds-siNA-052 | (SEQ ID NO: 56) | (SEQ ID NO: 57) | 0.008 | >1 |
| ds-siNA-053 | (SEQ ID NO: 56) | (SEQ ID NO: 58) | 0.004 | >1 |
| ds-siNA-054 | (SEQ ID NO: 56) | (SEQ ID NO: 59) | 0.034 | >1 |
| ds-siNA-055 | (SEQ ID NO: 56) | (SEQ ID NO: 60) | 0.007 | >1 |
| ds-siNA-056 | (SEQ ID NO: 54) | (SEQ ID NO: 29) | 0.037 | >1 |
| ds-siNA-057 | (SEQ ID NO: 55) | (SEQ ID NO: 29) | 0.035 | >1 |
| ds-siNA-058 | (SEQ ID NO: 54) | (SEQ ID NO: 57) | 0.041 | >1 |
| ds-siNA-059 | (SEQ ID NO: 54) | (SEQ ID NO: 58) | 0.009 | >1 |
| ds-siNA-060 | (SEQ ID NO: 54) | (SEQ ID NO: 59) | 0.78 | >1 |
| ds-siNA-061 | (SEQ ID NO: 54) | (SEQ ID NO: 60) | 0.061 | >1 |
| ds-siNA-062 | (SEQ ID NO: 55) | (SEQ ID NO: 57) | 0.109 | >1 |
| ds-siNA-063 | (SEQ ID NO: 55) | (SEQ ID NO: 58) | 0.027 | >1 |
| ds-siNA-064 | (SEQ ID NO: 55) | (SEQ ID NO: 59) | >1 | >1 |
| ds-siNA-065 | (SEQ ID NO: 55) | (SEQ ID NO: 60) | 0.084 | >1 |
| Control | (SEQ ID NO: 56) | (SEQ ID NO: 29) | 0.005 | >1 |
| ds-siNA-162 | | | | |

TABLE 25
siNA Comprising 2′-2ocp or 2′-omcp Nucleotides at the
3′- and 5′-ends of the Antisense Strand

| | | | EC₅₀ | CC₅₀ |
|---|---|---|---|---|
| Name | Sense | Antisense | (nM) | (nM) |
| ds-siNA-066 | (SEQ ID NO: 56) | (SEQ ID NO: 61) | 0.088 | >1 |
| ds-siNA-67 | (SEQ ID NO: 56) | (SEQ ID NO: 62) | 0.145 | >1 |
| ds-siNA-068 | (SEQ ID NO: 56) | (SEQ ID NO: 63) | >1 | >1 |
| ds-siNA-069 | (SEQ ID NO: 56) | (SEQ ID NO: 64) | >1 | >1 |
| Control | (SEQ ID NO: 11) | (SEQ ID NO: 29) | 0.01 | >1 |
| ds-siNA-150 | | | | |

TABLE 26
siNA Comprising 2′-ocp or 2′-omcp Nucleotides
in the 3′-overhang of the Antisense Strand

| | | | EC₅₀ | CC₅₀ |
|---|---|---|---|---|
| Name | Sense | Antisense | (nM) | (nM) |
| ds-siNA-070 | (SEQ ID NO: 1) | (SEQ ID NO: 65) | 0.077 | >1 |
| ds-siNA-071 | (SEQ ID NO: 1) | (SEQ ID NO: 66) | 0.012 | >1 |
| ds-siNA-072 | (SEQ ID NO: 1) | (SEQ ID NO: 67) | 0.166 | >1 |
| ds-siNA-073 | (SEQ ID NO: 1) | (SEQ ID NO: 68) | 0.179 | >1 |
| Control | (SEQ ID NO: 1) | (SEQ ID NO: 10) | 0.014 | >1 |
| ds-siNA-009 | | | | |

TABLE 27
Modified Duplex siNA Comprising High 2′-2ocp
or 2′-omcp Nucleotide Content

| | | | EC₅₀ | CC₅₀ |
|---|---|---|---|---|
| Name | Sense | Antisense | (nM) | (nM) |
| ds-siNA-074 | (SEQ ID NO: 69) | (SEQ ID NO: 29) | 0.092 | >1 |
| ds-siNA-075 | (SEQ ID NO: 53) | (SEQ ID NO: 70) | >1 | >1 |
| ds-siNA-076 | (SEQ ID NO: 69) | (SEQ ID NO: 70) | >1 | >1 |
| Control | (SEQ ID NO: 56) | (SEQ ID NO: 29) | 0.005 | >1 |
| ds-siNA-162 | | | | |

TABLE 28
siNA Comprising Modified Unlocked
Nucleotides on the Antisense Strand

| | | | EC₅₀ | CC₅₀ |
|---|---|---|---|---|
| Name | Sense | Antisense | (nM) | (nM) |
| ds-siNA-077 | (SEQ ID NO: 1) | (SEQ ID NO: 71) | 0.005 | >1 |
| ds-siNA-078 | (SEQ ID NO: 1) | (SEQ ID NO: 72) | 0.005 | >1 |
| Control | (SEQ ID NO: 1) | (SEQ ID NO: 88) | 0.036 | >1 |
| ds-siNA-159 | | | | |

TABLE 29
siNA Comprising Alternative 2′-Fluoro Nucleotide Pattern

| | | | EC₅₀ | CC₅₀ |
|---|---|---|---|---|
| Name | Sense | Antisense | (nM) | (nM) |
| ds-siNA-079 | (SEQ ID NO: 73) | (SEQ ID NO: 74) | 0.186 | >1 |
| ds-siNA-080 | (SEQ ID NO: 73) | (SEQ ID NO: 75) | 0.027 | >1 |
| ds-siNA-081 | (SEQ ID NO: 73) | (SEQ ID NO: 76) | 0.013 | >1 |
| ds-siNA-082 | (SEQ ID NO: 73) | (SEQ ID NO: 77) | >1 | >1 |
| ds-siNA-083 | (SEQ ID NO: 73) | (SEQ ID NO: 78) | >1 | >1 |
| ds-siNA-084 | (SEQ ID NO: 73) | (SEQ ID NO: 79) | 0.26 | >1 |
| Control | (SEQ ID NO: 1) | (SEQ ID NO: 88) | 0.115 | >1 |
| ds-siNA-159 | | | | |

TABLE 30
siNA Comprising 5′-cyclopropyl
Nucleotides on the Antisense Strand

| | | | EC₅₀ | CC₅₀ |
|---|---|---|---|---|
| Name | Sense | Antisense | (nM) | (nM) |
| ds-siNA-085 | (SEQ ID NO: 1) | (SEQ ID NO: 80) | >1 | >1 |
| ds-siNA-086 | (SEQ ID NO: 1) | (SEQ ID NO: 81) | 0.177 | >1 |
| ds-siNA-087 | (SEQ ID NO: 1) | (SEQ ID NO: 82) | >1 | >1 |
| ds-siNA-088 | (SEQ ID NO: 1) | (SEQ ID NO: 83) | 0.066 | >1 |
| ds-siNA-089 | (SEQ ID NO: 1) | (SEQ ID NO: 84) | >1 | >1 |
| ds-siNA-090 | (SEQ ID NO: 1) | (SEQ ID NO: 85) | 0.034 | >1 |
| ds-siNA-091 | (SEQ ID NO: 1) | (SEQ ID NO: 86) | >1 | >1 |
| ds-siNA-092 | (SEQ ID NO: 1) | (SEQ ID NO: 87) | >1 | >1 |
| Control | (SEQ ID NO: 1) | (SEQ ID NO: 10) | 0.014 | >1 |
| ds-siNA-009 | | | | |

TABLE 31
siNA Comprising 2′-F-3′-Xylo Modified Nucleotides
on the Sense or Antisense Strand

| | | | EC₅₀ | CC₅₀ |
|---|---|---|---|---|
| Name | Sense | Antisense | (pM) | (pM) |
| ds-siNA-093 | (SEQ ID NO: 98) | (SEQ ID NO: 79) | 11.69 | >1000 |
| ds-siNA-094 | (SEQ ID NO: 99) | (SEQ ID NO: 79) | 21.28 | >1000 |
| ds-siNA-095 | (SEQ ID NO: 100) | (SEQ ID NO: 79) | 31.49 | >1000 |
| ds-siNA-096 | (SEQ ID NO: 101) | (SEQ ID NO: 79) | 89.14 | >1000 |
| ds-siNA-097 | (SEQ ID NO: 102) | (SEQ ID NO: 79) | 18.22 | >1000 |
| ds-siNA-098 | (SEQ ID NO: 103) | (SEQ ID NO: 79) | 25.65 | >1000 |
| ds-siNA-099 | (SEQ ID NO: 73) | (SEQ ID NO: 104) | 496.63 | >1000 |
| ds-siNA-100 | (SEQ ID NO: 73) | (SEQ ID NO: 105) | 16.92 | >1000 |
| ds-siNA-101 | (SEQ ID NO: 73) | (SEQ ID NO: 106) | 24.61 | >1000 |
| ds-siNA-084 | (SEQ ID NO: 73) | (SEQ ID NO: 79) | 6.32 | >1000 |
| Control | (SEQ ID NO: 11) | (SEQ ID NO: 29) | 16.65 | >1000 |

TABLE 32
siNA Comprising 2′F Nucleotides on the Sense or Antisense Strand

| | | | EC₅₀ | CC₅₀ |
|---|---|---|---|---|
| Name | Sense | Antisense | (pM) | (pM) |
| ds-siNA-102 | (SEQ ID NO: 106) | (SEQ ID NO: 107) | 15.4 | >1000 |
| ds-siNA-103 | (SEQ ID NO: 73) | (SEQ ID NO: 108) | 8.0 | >1000 |
| ds-siNA-104 | (SEQ ID NO: 73) | (SEQ ID NO: 109) | 7.8 | >1000 |
| ds-siNA-105 | (SEQ ID NO: 73) | (SEQ ID NO: 110) | 7.5 | >1000 |
| ds-siNA-084 | (SEQ ID NO: 73) | (SEQ ID NO: 79) | 2.2 | >1000 |
| ds-siNA-106 | (SEQ ID NO: 73) | (SEQ ID NO: 111) | 6.7 | >1000 |
| ds-siNA-107 | (SEQ ID NO: 73) | (SEQ ID NO: 112) | 15.8 | >1000 |
| ds-siNA- 108 | (SEQ ID NO: 73) | (SEQ ID NO: 113) | 3.1 | >1000 |
| ds-siNA-109 | (SEQ ID NO: 73) | (SEQ ID NO: 114) | 11.9 | >1000 |
| ds-siNA-110 | (SEQ ID NO: 73) | (SEQ ID NO: 115) | 14.5 | >1000 |

TABLE 33
siNA Comprising Ganciclovir, Denvir, and 3′-ocp
Nucleotides on the Sense or Antisense Strand

| | | | EC₅₀ | CC₅₀ |
|---|---|---|---|---|
| Name | Sense | Antisense | (pM) | (pM) |
| ds-siNA-111 | (SEQ ID NO: 73) | (SEQ ID NO: 116) | 44.927 | >1000 |
| ds-siNA-112 | (SEQ ID NO: 73) | (SEQ ID NO: 117) | 35.201 | >1000 |
| ds-siNA-113 | (SEQ ID NO: 73) | (SEQ ID NO: 118) | 62.299 | >1000 |
| ds-siNA-114 | (SEQ ID NO: 73) | (SEQ ID NO: 119) | 133.268 | >1000 |
| ds-siNA-115 | (SEQ ID NO: 73) | (SEQ ID NO: 120) | 926.339 | >1000 |
| ds-siNA-116 | (SEQ ID NO: 73) | (SEQ ID NO: 121) | 74.215 | >1000 |
| ds-siNA-117 | (SEQ ID NO: 73) | (SEQ ID NO: 122) | >1000 | >1000 |
| ds-siNA-118 | (SEQ ID NO: 73) | (SEQ ID NO: 123) | 259.748 | >1000 |
| ds-siNA-119 | (SEQ ID NO: 73) | (SEQ ID NO: 124) | >1000 | >1 |
| ds-siNA-120 | (SEQ ID NO: 73) | (SEQ ID NO: 125) | >1000 | >1 |
| ds-siNA-159 | (SEQ ID NO: 1) | (SEQ ID NO: 88) | 66.267 | >1000 |
| ds-siNA- 084 | (SEQ ID NO: 73) | (SEQ ID NO: 79) | 33.519 | >1000 |
| Control | (SEQ ID NO: 1) | (SEQ ID NO: 10) | 29.962 | >1000 |
| ds-siNA-009 | | | | |
| Control | (SEQ ID NO: 165) | (SEQ ID NO: 152) | 50.933 | >1000 |
| ds-siNA-158 | | | | |

TABLE 34
siNA Ganciclovir and Denovir Modified Nucleotides on the Antisense Strand

| | | | siRNA | miRNA |
|---|---|---|---|---|
| | | | IC₅₀ | IC₅₀ |
| Name | Sense | Antisense | (nM) | (nM) |
| ds-siNA-111 | (SEQ ID NO: 73) | (SEQ ID NO: 116) | 0.0031 | >100 |
| ds-siNA-112 | (SEQ ID NO: 73) | (SEQ ID NO: 117) | 0.00398 | >100 |
| ds-siNA-113 | (SEQ ID NO: 73) | (SEQ ID NO: 118) | 0.0053 | >100 |
| ds-siNA-116 | (SEQ ID NO: 73) | (SEQ ID NO: 121) | 0.0011 | >100 |
| ds-siNA-084 | (SEQ ID NO: 73) | (SEQ ID NO: 79) | 0.0012 | 0.307 |

TABLE 35
siRNA G analogs into with vinyl phosphate 5′ end-caps

| | | | siRNA | miRNA |
|---|---|---|---|---|
| | | | IC₅₀ | IC₅₀ |
| Name | Sense | Antisense | (nM) | (nM) |
| ds-siNA-147 | (SEQ ID NO: 56) | (SEQ ID NO: 153) | 0.001 | 0.739 |
| ds-siNA-148 | (SEQ ID NO: 56) | (SEQ ID NO: 154) | 0.003 | 0.964 |
| ds-siRN-149 | (SEQ ID NO: 56) | (SEQ ID NO: 155) | 0.004 | 0.08 |
| Control | (SEQ ID NO: 11) | (SEQ ID NO: 29) | 0.023 | 0.49 |
| Ds-siNA-150 | | | | |

TABLE 36
siNA Comprising 2′-OMe-3′-Xylo Modified Nucleotides on the Antisense Strand

| | | | EC₅₀ | CC₅₀ |
|---|---|---|---|---|
| Name | Sense | Antisense | (pM) | (pM) |
| ds-siNA-121 | (SEQ ID NO: 73) | (SEQ ID NO: 126) | >1000 | >1000 |
| ds-siNA- 122 | (SEQ ID NO: 73) | (SEQ ID NO: 127) | >1000 | >1000 |
| ds-siNA- 123 | (SEQ ID NO: 73) | (SEQ ID NO: 128) | 199.645 | >1000 |
| ds-siNA- 124 | (SEQ ID NO: 73) | (SEQ ID NO: 129) | 150.736 | >1000 |
| ds-siNA- 125 | (SEQ ID NO: 73) | (SEQ ID NO: 130) | 28.657 | >1000 |
| ds-siNA- 126 | (SEQ ID NO: 73) | (SEQ ID NO: 131) | >1000 | >1000 |
| ds-siNA- 127 | (SEQ ID NO: 73) | (SEQ ID NO: 132) | 223.176 | >1000 |
| ds-siNA-159 | (SEQ ID NO: 1) | (SEQ ID NO: 88) | 66.267 | >1000 |
| ds-siNA-084 | (SEQ ID NO: 73) | (SEQ ID NO: 79) | 33.519 | >1000 |
| ds-siNA-009 | (SEQ ID NO: 1) | (SEQ ID NO: 10) | 29.962 | >1000 |
| Control | (SEQ ID NO: 165) | (SEQ ID NO: 152) | 50.933 | >1000 |
| ds-siNA-158 | | | | |

TABLE 37
Additional si-NA Comprising 2′-OMe-3′-Xylo
Modified Nucleotides on the Antisense

| | | | EC₅₀ | CC₅₀ |
|---|---|---|---|---|
| Name | Sense | Antisense | (pM) | (pM) |
| ds-siNA-128 | (SEQ ID NO: 1) | (SEQ ID NO: 133) | >1000 | >1000 |
| ds-siNA-129 | (SEQ ID NO: 1) | (SEQ ID NO: 134) | >1000 | >1000 |
| ds-siNA-130 | (SEQ ID NO: 1) | (SEQ ID NO: 135) | >1000 | >1000 |
| ds-siNA-131 | (SEQ ID NO: 1) | (SEQ ID NO: 136) | 23.99 | >1000 |
| ds-siNA-159 | (SEQ ID NO: 1) | (SEQ ID NO: 88) | 105.41 | >1000 |
| Control | (SEQ ID NO: 11) | (SEQ ID NO: 29) | 62 | >1000 |
| ds-siNA-150 | | | | |

TABLE 38
siNA Comprising 2′-ocp, 2′-omcp, and vmX Nucleotides

| | | | EC₅₀ | CC₅₀ |
|---|---|---|---|---|
| Name | Sense | Antisense | (nM) | (nM) |
| ds-siNA- 132 | (SEQ ID NO: 1) | (SEQ ID NO: 137) | 0.017 | >1 |
| ds-siNA-133 | (SEQ ID NO: 1) | (SEQ ID NO: 138) | 0.003 | >1 |
| ds-siNA- 134 | (SEQ ID NO: 1) | (SEQ ID NO: 139) | 0.051 | >1 |
| ds-siNA- 135 | (SEQ ID NO: 1) | (SEQ ID NO: 140) | 0.026 | >1 |
| ds-siNA- 136 | (SEQ ID NO: 1) | (SEQ ID NO: 141) | 0.037 | >1 |
| ds-siNA- 137 | (SEQ ID NO: 1) | (SEQ ID NO: 142) | >1 | >1 |
| ds-siNA- 138 | (SEQ ID NO: 1) | (SEQ ID NO: 143) | 0.035 | >1 |
| ds-siNA- 139 | (SEQ ID NO: 1) | (SEQ ID NO: 144) | 0.398 | >1 |
| ds-siNA- 140 | (SEQ ID NO: 1) | (SEQ ID NO: 145) | >1 | >1 |
| ds-siNA-141 | (SEQ ID NO: 1) | (SEQ ID NO: 146) | >1 | >1 |
| ds-siNA-142 | (SEQ ID NO: 1) | (SEQ ID NO: 147) | >1 | >1 |
| ds-siNA-143 | (SEQ ID NO: 56) | (SEQ ID NO: 149) | >1 | >1 |
| ds-siNA-144 | (SEQ ID NO: 56) | (SEQ ID NO: 148) | >1 | >1 |
| ds-siNA-145 | (SEQ ID NO: 56) | (SEQ ID NO: 149) | >1 | >1 |
| ds-siNA-146 | (SEQ ID NO: 56) | (SEQ ID NO: 150) | >1 | >1 |
| Control | (SEQ ID NO: 165) | (SEQ ID NO: 152) | 0.008 | >1 |
| ds-siNA-158 | | | | |
| Reference | (SEQ ID NO: 1) | (SEQ ID NO: 10) | 0.015 | >1 |
| ds-siNA-009 | | | | |

TABLE 39
siRNA with TNA phosphonate chemistry

| | | | EC₅₀ | CC₅₀ |
|---|---|---|---|---|
| Name | Sense | Antisense | (nM) | (nM) |
| ds-siNA-151 | (SEQ ID NO: 56) | (SEQ ID NO: 156) | 0.007 | >1 |

TABLE 40
siRNA Comprising Stereodefined PS linkages

| | | | EC₅₀ | CC₅₀ |
|---|---|---|---|---|
| Name | Sense | Antisense | (nM) | (nM) |
| ds-siNA-156 | (SEQ ID NO: 56) | (SEQ ID NO: 161) | 0.002 | >1 |
| ds-siNA-157 | (SEQ ID NO: 56) | (SEQ ID NO: 162) | 0.002 | >1 |
| ds-siNA-150 | (SEQ ID NO: 11) | (SEQ ID NO: 29) | 0.01 | >1 |
| ds-siNA-162 | (SEQ ID NO: 56) | (SEQ ID NO: 29) | 0.004 | >1 |
| ds-siNA-158 | (SEQ ID NO: 165) | (SEQ ID NO: 152) | 0.022 | >1 |

### Example 5—In Vivo Activity of Ds-siNAs Comprising a 5′Vinyl Phosphonate Moiety and Modified Unlocked Nucleotides

Mice were infected with AAV-HBV on day-28 of the study. The test ds-siNAs, ds-siNA-159, ds-siNA-160, ds-siNA-077, ds-siNA-078, or ds-siNA-161 negative control ds-siNA were dosed subcutaneously as single dose on days 0 at 5 mg/kg. Serial blood collections were taken on day 0, 7, 14, 21, and 28. Serum HBsAg was assayed through ELISA.

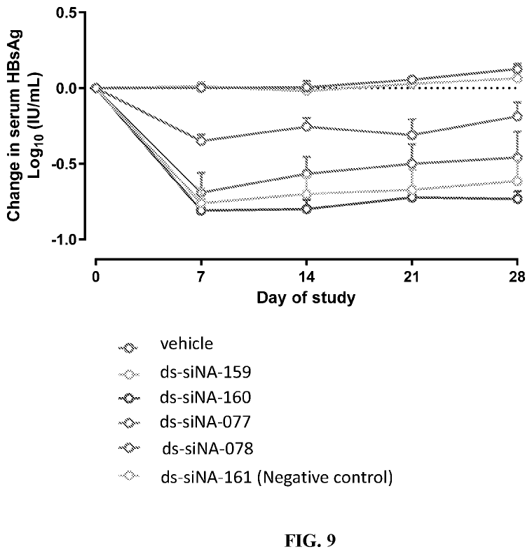

As shown in FIG. 9, presence of the 5′vinyl phosphonate moiety and modified unlocked nucleotides on the antisense strand of ds-siNA-077, ds-siNA-078 caused reduced activity compared to their counter parts ds-siNAs ds-siNA-159, ds-siNA-160, which lacked such modifications.

### Example 6—In Vitro Stability and In Vivo Activity of Ds-siNAs Comprising 5′-Cyclopropyl Nucleotides

The stability of ds-siNA-085, ds-siNA-086, ds-siNA-087, ds-siNA-088, ds siNA-089, ds-siNA-090, ds-siNA-091, ds-siNA-092, ds-siNA-159, were measured in mouse liver homogenates. Mouse livers were ground and then 50 mg of the ground liver was transferred to each microcentrifuge tube set in dry ice. Homogenization buffer (50 mM Tris. HCl, 150 mM KCl, pH 7.2) was added to a concentration of 200 mg/ml. Microcentrifuge tubes were placed into a heated multi-shaker and shaken at 40° C. and 1,200 rpm for one hour. For each mL of homogenate, 40 μL 100 mM MgCl2 and 40 μL 100x antibiotic was added. Liver homogenate was stored at −20° C. On the day of experiment, the required volume of liver homogenate was thawed and added to oligos (final concentration of oligo in matrix was 5 μM). The microcentrifuge tubes were incubated at 37° C. with gentle shaking (˜400 rpm) using a heated shaker. At the end of each timepoint (e.g. 48 hours), 20 μL of an internal standard (2,000 ng/mL in nuclease-free water), 200 μL of 10% phosphoric acid and 600 μL of Clarity lysis-loading buffer was added to the microcentrifuge tubes with vertexing to mix after each addition. Solid phase extraction was then performed.

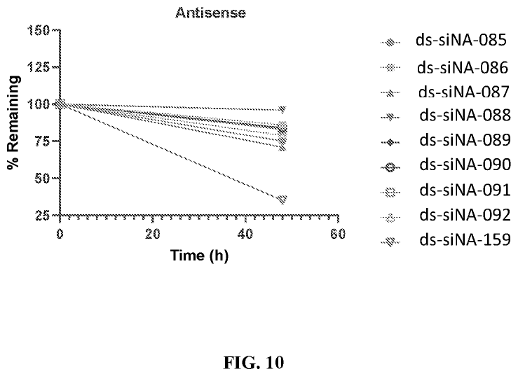

As shown in FIG. 10, ds-siNA-088 and ds-siNA-090 demonstrated the highest stability after 48 hours. Accordingly, ds-siNA-088 and ds-siNA-090 were selected for in vivo activity analysis. Mice were infected with AAV-HBV on day-28 of the study. The test ds-siNAs, ds-siNA-088 and ds-siNA-090, positive control ds-siNA-159, or vehicle were dosed subcutaneously as single dose on days 0 at 5 mg/kg. Serial blood collections were taken on day 0, 7, 14, 21, and 28. Serum HBsAg was assayed through ELISA.

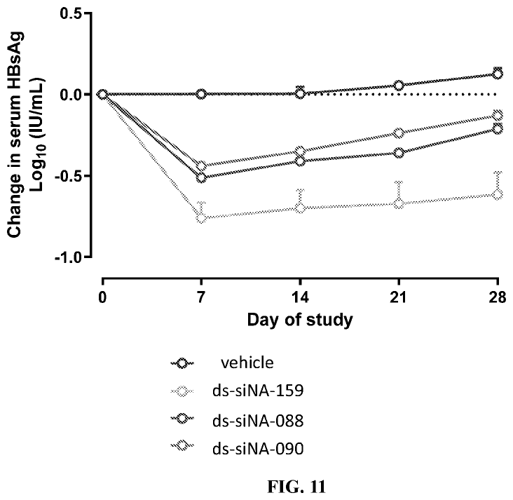

As shown in FIG. 11, higher durability and in vitro activity of ds-siNA-088 and ds-siNA-090 did not translate to in vivo performance, with HBsAg levels moderately higher than the positive control.

### Example 7—In Vivo Activity of Ds-siNAs Comprising 3′OH and Unlocked Modified Nucleotides on the Antisense Strand

Mice were infected with AAV-HBV on day-28 of the study. The test ds-siNAs, ds-siNA-080 and ds-siNA-081, negative control ds-siNA-083, positive control ds-siNA-084, or vehicle were dosed subcutaneously as single dose on days 0 at 5 mg/kg. Serial blood collections were taken on day 0, 7, 14, 21, and 28. Serum HBsAg was assayed through ELISA.

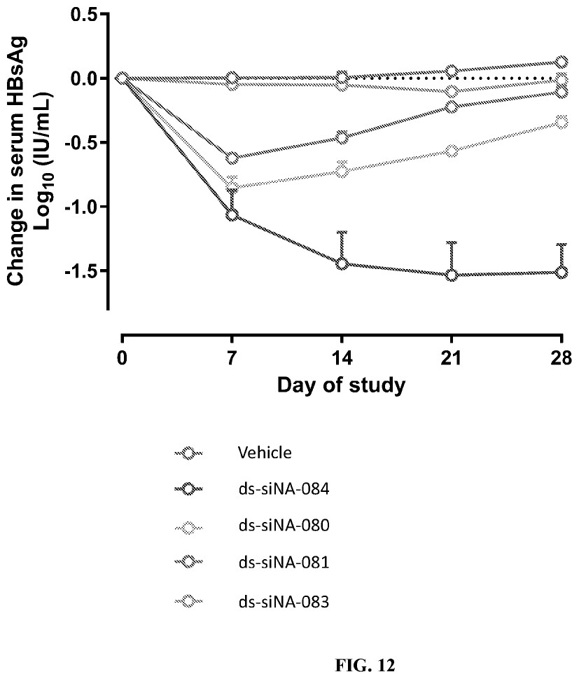

As shown in FIG. 12, presence of the 3′OH and unlocked modified nucleotides on the antisense strand of ds-siNA-080, ds-siNA-081 significantly reduced in vivo activity.

### Example 8—In Vivo Activity of Ds-siNAs Comprising a 5′-End Cap on the Antisense Strand

Mice were infected with AAV-HBV on day-28 of the study. The test ds-siNA ds-siNA-162, control ds-siNA-151, or vehicle were dosed subcutaneously as single dose on days 0 at 5 mg/kg. Serial blood collections were taken on day 0, 7, 14, 21, and 28. Serum HBsAg was assayed through ELISA.

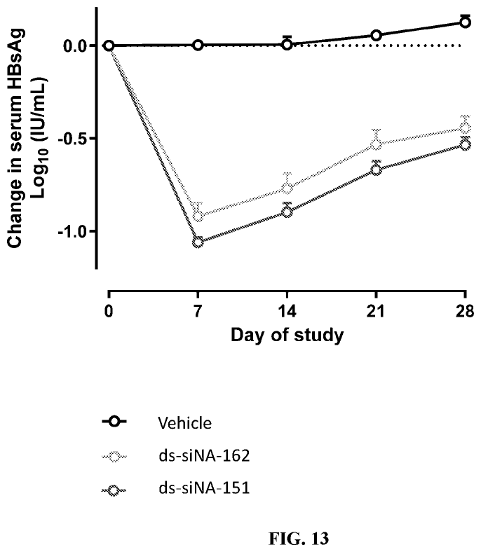

As shown in FIG. 13, the presence of the 5′-end cap on the antisense strand of ds-siNA-162 provided similar in vivo activity as compared with the ds-siNA-151, which does not comprise a 5′-end cap.

### Example 9—Comparison of In Vivo Activity of Ds-siNA Analogues

The stability of ds-siNA-159, a ds-siNA-159 analogue having a single nucleotide modification in the 3′ overhang of the antisense strand, ds-siNA-009, were measured in mouse liver homogenates. Mouse livers were ground and then 50 mg of the ground liver was transferred to each microcentrifuge tube set in dry ice. Homogenization buffer (50 mM Tris. HCl, 150 mM KCl, pH 7.2) was added to a concentration of 200 mg/ml. Microcentrifuge tubes were placed into a heated multi-shaker and shaken at 40° C. and 1,200 rpm for one hour. For each mL of homogenate, 40 μL 100 mM MgCl2 and 40 μL 100× antibiotic was added. Liver homogenate was stored at −20° C. On the day of experiment, the required volume of liver homogenate was thawed and added to oligos (final concentration of oligo in matrix was 5 μM). The microcentrifuge tubes were incubated at 37° C. with gentle shaking (˜400 rpm) using a heated shaker. At the end of each timepoint (e.g. 48 hours), 20 μL of an internal standard (2,000 ng/mL in nuclease-free water), 200 μL of 10% phosphoric acid and 600 μL of Clarity lysis-loading buffer was added to the microcentrifuge tubes with vertexing to mix after each addition. Solid phase extraction was then performed.

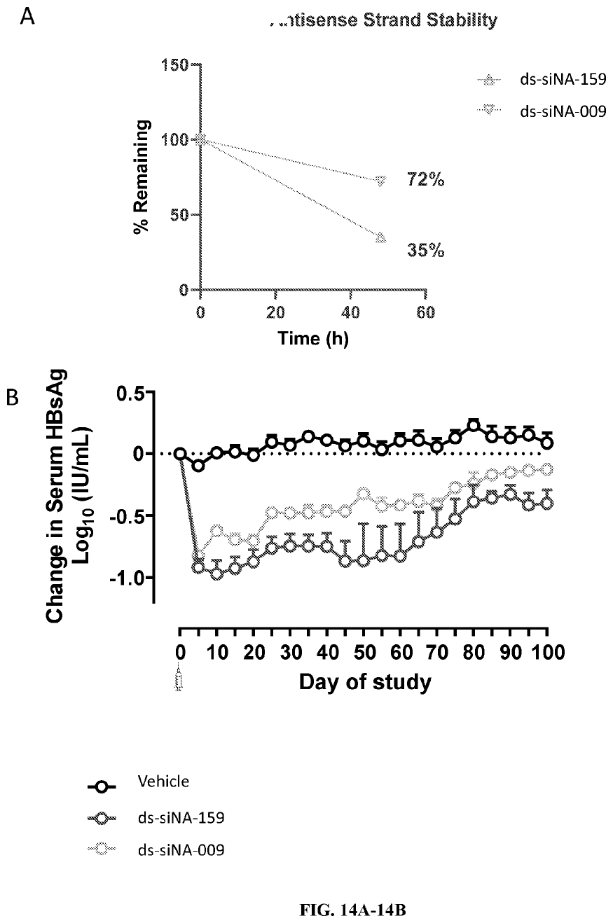

As shown in FIG. 14A, ds-siNA-009 demonstrated improved stability compared to the parent ds-siNA-159 after 48 hours. To determine if the increased in vitro stability translated into improved in vivo activity, in vivo activity analysis was performed.

Mice were infected with AAV-HBV on day-28 of the study. The test ds-siNAs ds-siNA-159, ds-siNA-009, or vehicle were dosed subcutaneously as single dose on days 0 at 5 mg/kg. Serial blood collections were taken on day 0, 5, and every 5 days thereafter up to 100 days. Serum HBsAg was assayed through ELISA.

As shown in FIG. 14B, ds-siNA-009 exhibited moderately reduced in vivo activity compared to the parent ds-siNA-159.

### Example 10—In Vitro Stability and In Vivo Activity of Ds-siNAs Comprising Xylo Modified Nucleotides

The stability of ds-siNA-131 was measured in mouse liver homogenates. Mouse livers were ground and then 50 mg of the ground liver was transferred to each microcentrifuge tube set in dry ice. Homogenization buffer (50 mM Tris·HCl, 150 mM KCl, pH 7.2) was added to a concentration of 200 mg/ml. Microcentrifuge tubes were placed into a heated multi-shaker and shaken at 40° C. and 1,200 rpm for one hour. For each mL of homogenate, 40 μL 100 mM MgCl2 and 40 μL 100x antibiotic was added. Liver homogenate was stored at −20° C. On the day of experiment, the required volume of liver homogenate was thawed and added to oligos (final concentration of oligo in matrix was 5 μM). The microcentrifuge tubes were incubated at 37° C. with gentle shaking (˜400 rpm) using a heated shaker. At the end of each timepoint (e.g. 48 hours), 20 μL of an internal standard (2,000 ng/mL in nuclease-free water), 200 μL of 10% phosphoric acid and 600 μL of Clarity lysis-loading buffer was added to the microcentrifuge tubes with vertexing to mix after each addition. Solid phase extraction was then performed.

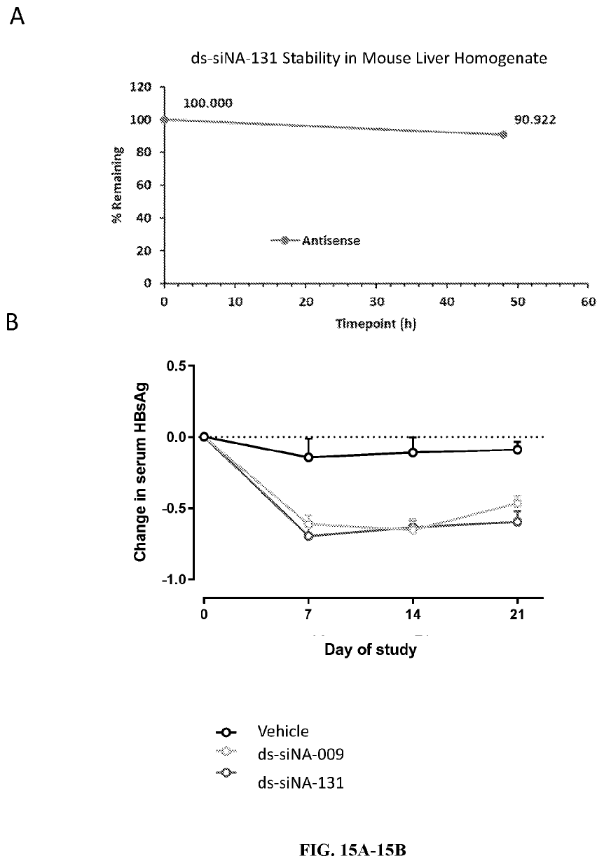

As shown in FIG. 15A, ds-siNA-131 demonstrated increased stability after 48 hours, compared to ds-siNA-159 and ds-siNA-009 in FIG. 14A. Accordingly, ds-siNA-131 was selected for in vivo activity analysis. Mice were infected with AAV-HBV on day-28 of the study. The test ds-siNA ds-siNA-131, ds-siNA-009, or vehicle were dosed subcutaneously as single dose on days 0 at 5 mg/kg. Serial blood collections were taken on day 0, 7, 14, and 21. Serum HBsAg was assayed through ELISA.

As shown in FIG. 15B, increased stability of ds-siNA-131 translates to in vivo activity, comparable to that of ds-siNA-009.

### Example 11—In Vivo Activity and In Vitro Stability of Ds-siNAs Comprising 2′F Modified Nucleotides

Mice were infected with AAV-HBV on day-28 of the study. The test ds-siNAs ds-siNA-103, ds-siNA-104, ds-siNA-105, ds-siNA-084, ds-siNA-106, ds-siNA-108, ds-siNA-109, or vehicle were dosed subcutaneously as single dose on days 0 at 5 mg/kg. Serial blood collections were taken on day 0, 5, and every 5 days thereafter up to 25 days. Serum HBsAg was assayed through ELISA.

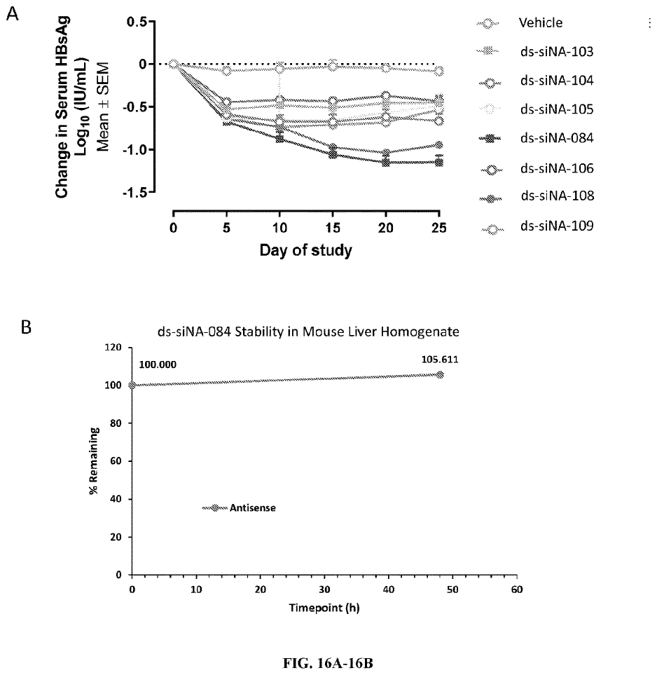

As shown in FIG. 16A, ds-siNA-084 exhibited the best in vivo activity compared to the test ds-siNAs. Accordingly, in vitro stability of ds-siNA-084 was assayed in mouse liver homogenate according to previously described methods. As can be seen in FIG. 16B, ds-siNA-084 demonstrated increased stability after 48 hours, compared to ds-siNA-159 and ds-siNA-009 in FIG. 15A.

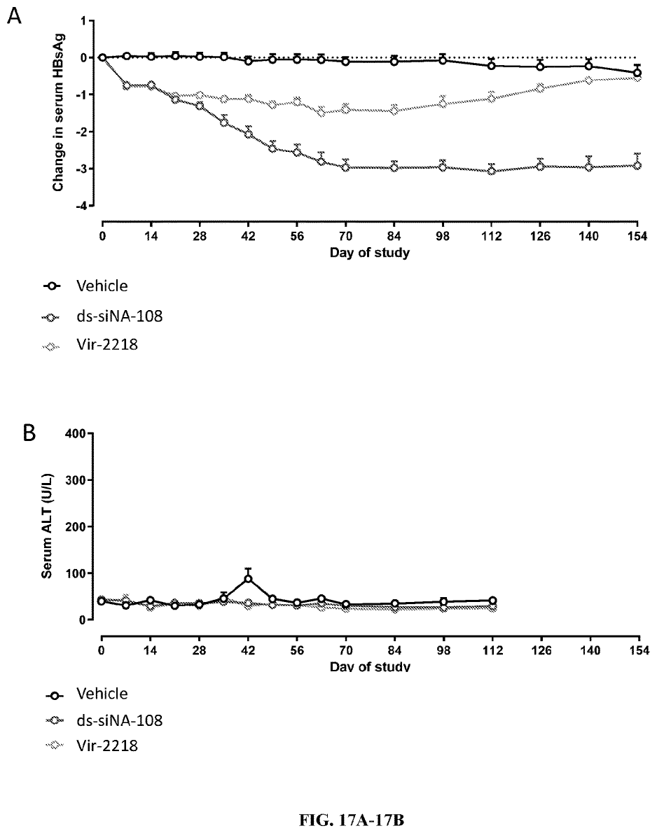

The efficacy of ds-siNA-108, which exhibited the second best in vivo activity in FIG. 17A, was measured compared to Vir-2218. The test ds-siNAs ds-siNA-108, Vir-2218, or vehicle were dosed subcutaneously as single dose on days 0 and every 14 days thereafter for 70 days at 5 mg/kg. Serial blood collections were taken on day 0 and every 7 days thereafter up to 164 days. Serum HBsAg, HBeAg and alanine amino transferase (ALT) levels were assayed through ELISA.

As shown in FIG. 17, ds-siNA-108 demonstrated significantly increased activity compared to Vir-2218 as shown by (A) HBsAg. Serum ALT levels were comparable between the test ds-siNAs ds-siNA-108 and Vir-2218 as shown in (B).

### Example 12—Comparison of In Vivo Activity of Ds-siNA Analogues and HBV Treatment Vir-2218

Mice were infected with AAV-HBV on day-28 of the study. The test ds-siNAs ds-siNA-159, ds-siNA-084, Vir-2218, or vehicle were dosed subcutaneously as single dose on days 0 and every 14 days thereafter for 70 days at 5 mg/kg. Serial blood collections were taken on day 0 and every 7 days thereafter up to 168 days. Serum HBsAg, HBeAg and alanine amino transferase (ALT) levels were assayed through ELISA.

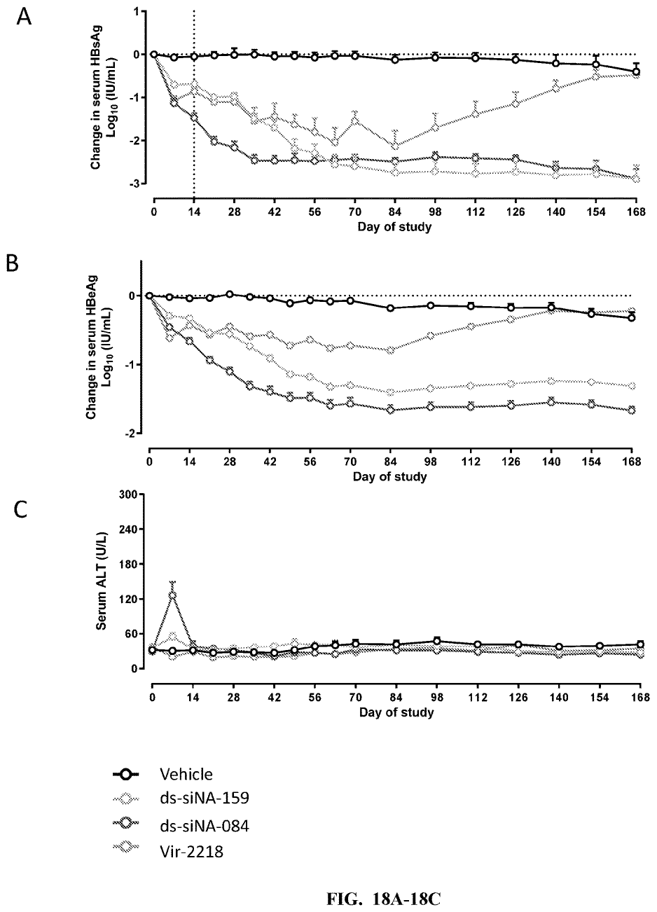

As shown in FIG. 18, ds-siNA-159 and ds-siNA-084 demonstrated increased activity compared to Vir-2218 as shown by (A) HBsAg and (B) HBeAg levels. Serum ALT levels were comparable between the test ds-siNAs ds-siNA-159 and Vir-2218 as shown in (C). ds-siNA-084 showed an ALT flare after first dose on day 0 and then returned to normal on Day 14. It did not cause ALT elevation again even with more repeat dosing.

### Example 13—10× Efficacious Dose of Ds-siNA does not Result in ALT Signal in Non-Infected Mice

To measure ALT, a very sensitive marker for liver toxicity cause by drugs, non-infected mice were dosed subcutaneously with test ds-siNAs ds-siNA-084 on day 0 at either 5 mg/kg, 15 mg/kg, 50 mg/kg or a Control siNA at either 5 mg/kg or 15 mg/kg. Serial blood collections were taken on day 0, 7, and 14. Serum ALT levels were assayed through ELISA.

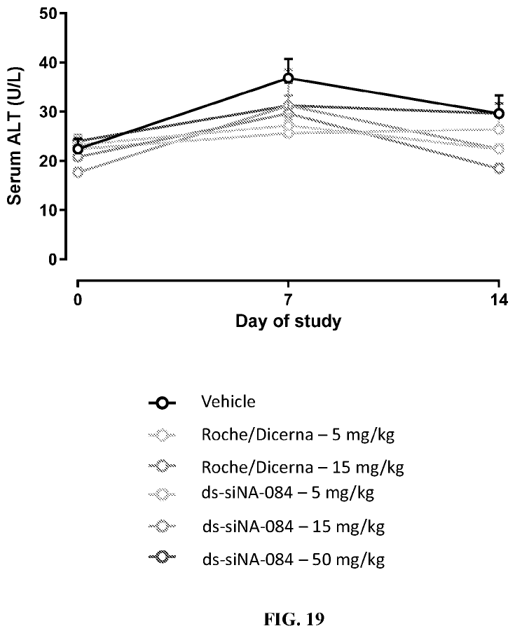

As shown in FIG. 19, ALT levels were not significantly affected by increased dosages of either siNA-084 or Roche/Discerna at 10X efficacious dose. Comparing FIGS. 18C and 19, ds-siNA-084 only showed ALT in AAV-HBV infected mice, suggesting ALT might be immune-related which could occur when siNA-084 activated mouse CD8+ T cell to clear infected hepatocytes.

### Example 14—In Vivo Activity of Ganciclovir and Denavir Modified Ds-siNA

Mice were infected with AAV-HBV on day-28 of the study. The test ds-siNAs ds-siNA-111, ds-siNA-112, ds-siNA-113, ds-siNA-116, control ds-siNA-084, or vehicle were dosed subcutaneously as single dose on day 0 at 5 mg/kg. Serial blood collections were taken on day 0, 7, 14, and 21. Serum HBsAg and ALT levels were assayed through ELISA.

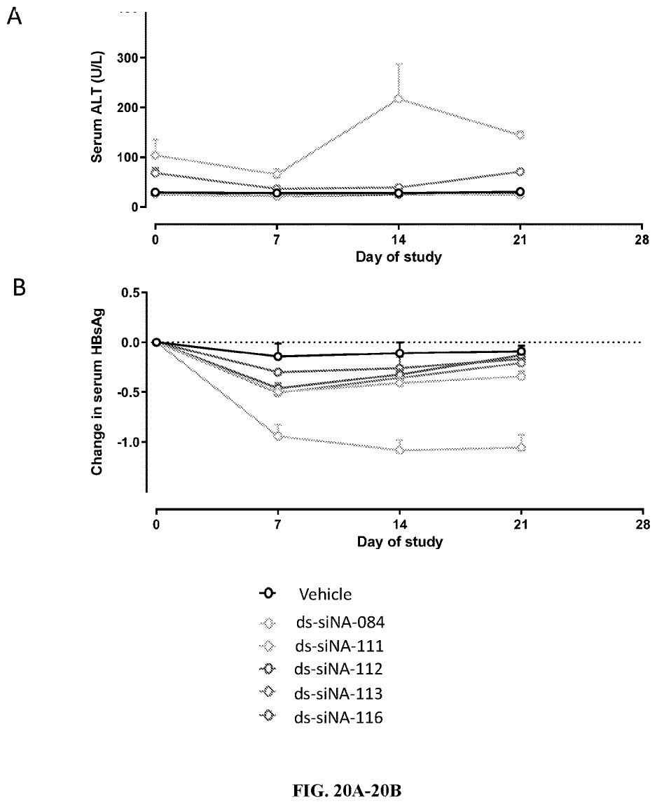

As shown in FIG. 20, ds-siNA-111, ds-siNA-112, ds-siNA-113, ds-siNA-116 reduced ALT levels (A) but lost significant potency as shown by HBsAg levels (B).

### Example 15—In Vitro Stability and In Vivo Activity of Ds-siNAs Comprising Xylo Modified Nucleotides

The stability of parent ds-siNA-084 and xylo modified ds-siNA-125 were measured in mouse liver homogenates. Mouse livers were ground and then 50 mg of the ground liver was transferred to each microcentrifuge tube set in dry ice. Homogenization buffer (50 mM Tris·HCl, 150 mM KCl, pH 7.2) was added to a concentration of 200 mg/ml. Microcentrifuge tubes were placed into a heated multi-shaker and shaken at 40° C. and 1,200 rpm for one hour. For each mL of homogenate, 40 μL 100 mM MgCl2 and 40 μL 100x antibiotic was added. Liver homogenate was stored at −20° C. On the day of experiment, the required volume of liver homogenate was thawed and added to oligos (final concentration of oligo in matrix was 5 μM). The microcentrifuge tubes were incubated at 37° C. with gentle shaking (˜400 rpm) using a heated shaker. At the end of each timepoint (e.g. 48 hours), 20 μL of an internal standard (2,000 ng/mL in nuclease-free water), 200 μL of 10% phosphoric acid and 600 μL of Clarity lysis-loading buffer was added to the microcentrifuge tubes with vertexing to mix after each addition. Solid phase extraction was then performed.

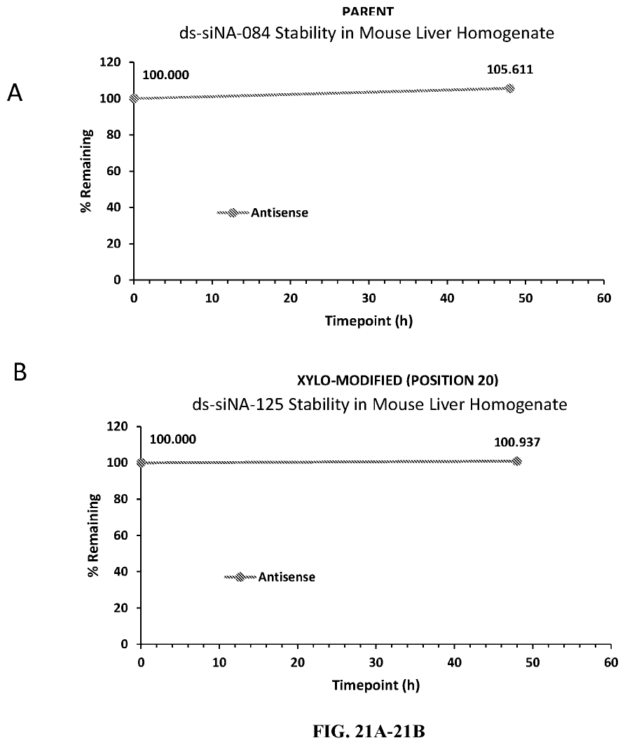

As shown in FIG. 21, parent ds-siNA-084 (A) and xylo modified ds-siNA-125 (B) demonstrated similar stability after 48 hours. ds-siNA-084 and ds-siNA-125 were analyzed for in vivo activity. Mice were infected with AAV-HBV on day-28 of the study. ds-siNA-084, ds-siNA-125, or vehicle were dosed subcutaneously as single dose on days 0 at 5 mg/kg. Serial blood collections were taken on day 0, 7, 14, and 21. Serum HBsAg and ALT were assayed through ELISA.

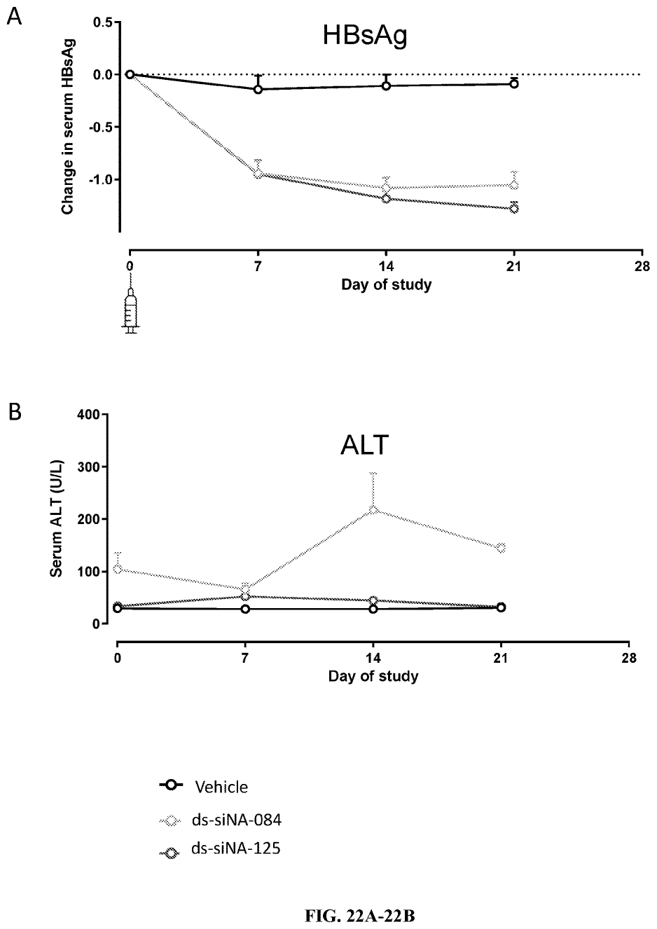

As shown in FIG. 22A, ds-siNA-125 maintained potency compared to ds-siNA-084, whilst reducing ALT (FIG. 22B).

### Example 16—In Vitro Stability and In Vivo Activity of Ds-siNAs Comprising Stereodefined PS Linkages

The stability of parent ds-siNA-143, ds-siNA-157 modified with a PS(S) linkage, and ds-siNA-156 modified with a PS(R) linkage were measured in mouse liver homogenates. Mouse livers were ground and then 50 mg of the ground liver was transferred to each microcentrifuge tube set in dry ice. Homogenization buffer (50 mM Tris·HCl, 150 mM KCl, pH 7.2) was added to a concentration of 200 mg/ml. Microcentrifuge tubes were placed into a heated multi-shaker and shaken at 40° C. and 1,200 rpm for one hour. For each mL of homogenate, 40 μL 100 mM MgCl2 and 40 μL 100x antibiotic was added. Liver homogenate was stored at −20° C. On the day of experiment, the required volume of liver homogenate was thawed and added to oligos (final concentration of oligo in matrix was 5 μM). The microcentrifuge tubes were incubated at 37° C. with gentle shaking (˜400 rpm) using a heated shaker. At the end of each timepoint (e.g. 48 hours), 20 μL of an internal standard (2,000 ng/mL in nuclease-free water), 200 μL of 10% phosphoric acid and 600 μL of Clarity lysis-loading buffer was added to the microcentrifuge tubes with vertexing to mix after each addition. Solid phase extraction was then performed.

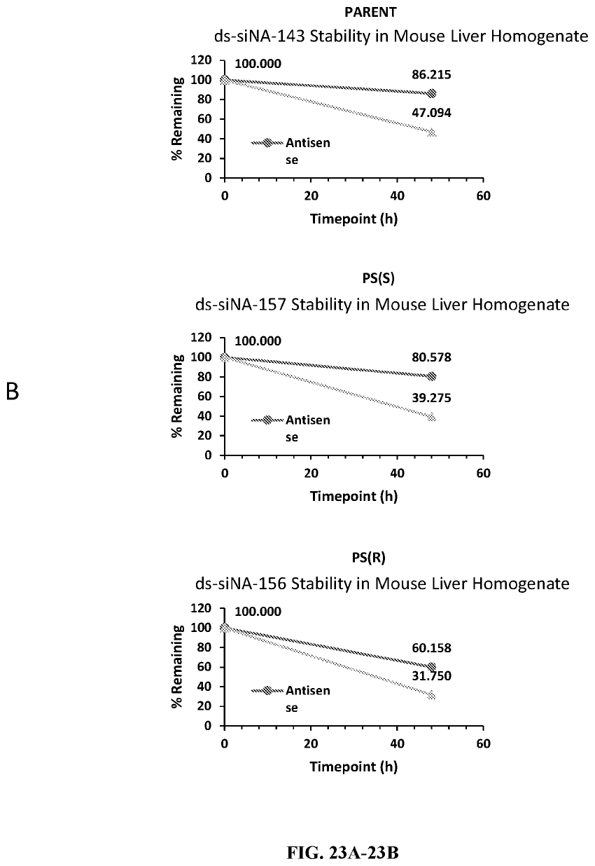

FIG. 23 shows stability of the sense and antisense strands of (A) parent ds-siNA-143, (B) ds-siNA-157 modified with a PS(S) linkage, and (C) ds-siNA-156 modified with a PS(R) linkage. Stability of the antisense strand of the parent ds-siNA-143 and ds-siNA-157 modified with a PS(S) linkage are comparable, and that of the ds-siNA-156 modified with a PS(R) linkage is less stable. Sense strands of all ds-siNAs included in the assay degraded within 48 h.

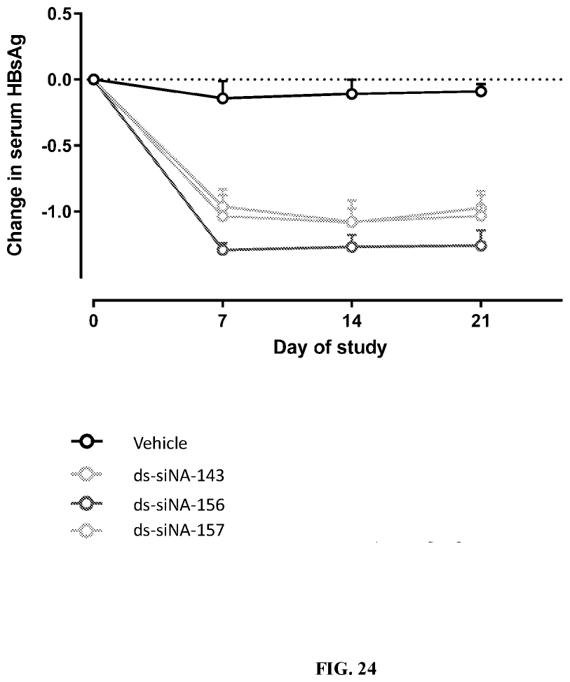

Each of the ds-siNAs were tested for in vivo activity. Mice were infected with AAV-HBV on day-28 of the study. ds-siNA-143, ds-siNA-157, ds-siNA-156, or vehicle were dosed subcutaneously as single dose on days 0 at 5 mg/kg. Serial blood collections were taken on day 0, 7, 14, and 21. Serum HBsAg was assayed through ELISA. Results show that the ds-siNA-156 modified with a PS(R) linkage showed a slight improvement in activity over the parent and s-siNA-157 modified with a PS(S) linkage (FIG. 24).

### Example 17—In Vivo Activity of Ds-siNA Comprising Denavir(s) and Mun12 Modified Nucleotides

Mice were infected with AAV-HBV on day-28 of the study. The test ds-siNAs ds-siNA-147, ds-siNA-148, parent ds-siNA-149, or vehicle were dosed subcutaneously as single dose on day 0 at 5 mg/kg. Serial blood collections were taken on day 0, 7, 14, and 21. Serum HBsAg was assayed through ELISA.

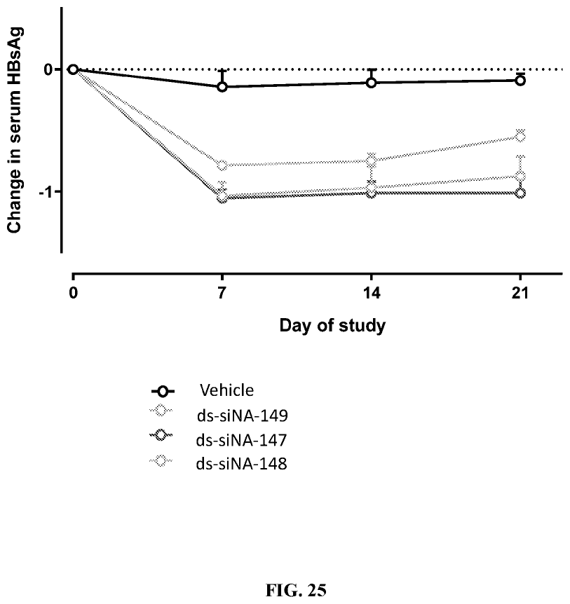

As shown in FIG. 25, ds-siNA-147, shown to improve off target profile in Table 35, maintained potency, with ds-siNA-148 having reduced potency.

### Example 18: Preparation of Compound 40-9 (GalNAc4 Amidite)

Compound 40-9 can be conjugated to any siNA disclosed herein as a targeting moiety. This compound, pictured below, can be prepared according to the following brief description.

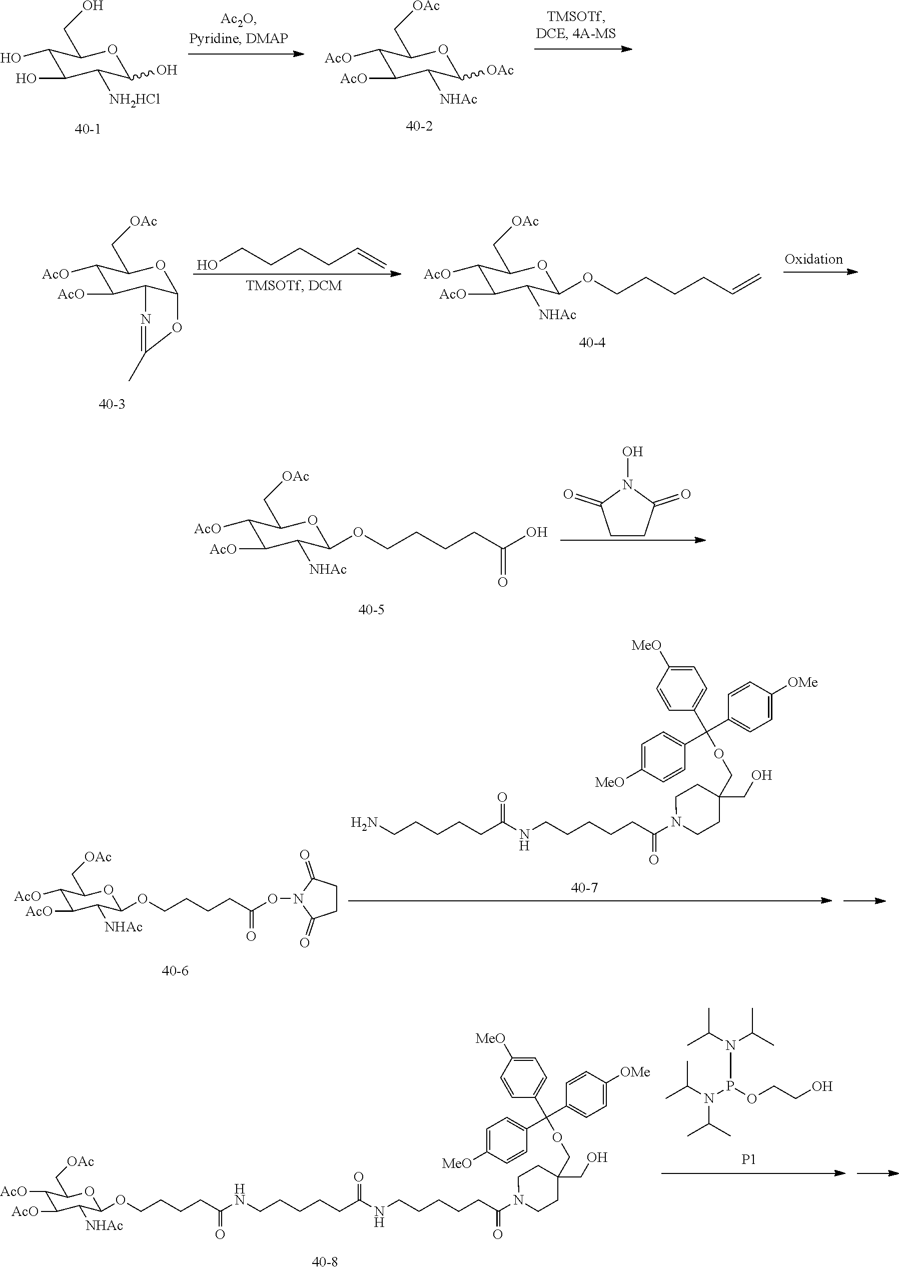

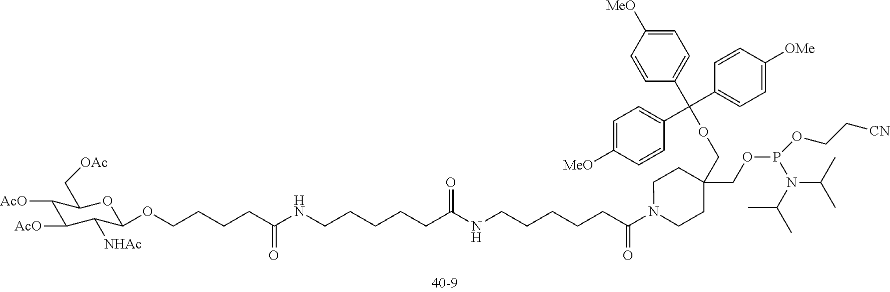 [SMILES: COc1ccc(C(OCC2(COP(OCCC#N)N(C(C)C)C(C)C)CCN(C(=O)CCCCCNC(=O)CCCCCNC(=O)CCCCOC3OC(COC(C)=O)C(OC(C)=O)C(OC(C)=O)C3NC(C)=O)CC2)(c2ccc(OC)cc2)c2ccc(OC)cc2)cc1]

The building block compound 40-9 is useful for making embodiments of modified phosphorothioated oligonucleotides. The compound 40-9 was prepared as follows:

Preparation of compound 40-2: To a solution of commercially available glucosamine hydrochloride 40-1 (60 g, 278.25 mmol, 1 eq) in DCM (300 mL) at 0° C. was added Ac₂O (323.83 g, 3.17 mol, 297.09 mL, 11.4 eq) dropwise, followed by pyridine (300 mL) and DMAP (3.40 g, 27.83 mmol, 0.1 eq). The mixture was allowed to gradually warm to 20° C. and stirred at 20° C. for 24 hours. Upon completion as monitored by LCMS, the mixture was concentrated under reduced pressure, diluted with DCM (900 mL), and extracted with NaHCO₃ (sat., aqueous 300 mL*3). The combined organic layers were washed with brine (300 mL), dried over Na₂SO₄, filtered and concentrated under reduced pressure to give compound 40-2 (89.5 g, crude) as a yellow solid. ¹H NMR (400 MHz, CDCl₃) δ=6.16 (d, J=3.8 Hz, 1H), 5.62 (d, J=9.0 Hz, 1H), 5.27-5.16 (m, 2H), 4.54-4.43 (m, 1H), 4.24 (dd, J=4.0, 12.5 Hz, 1H), 4.10-3.94 (m, 2H), 2.18 (s, 3H), 2.08 (s, 3H), 2.04 (d, J=4.0 Hz, 6H), 1.93 (s, 3H; LCMS (ESI): m/z calcd. for C₁₆H₂₃NaNO₁₀ 412.34 [M+Na]⁺, found 412.0).

Preparation of compound 40-3: To a solution of compound 40-2 (40 g, 102.73 mmol, 1 eq) in DCE (320 mL) at 25° C. was added dropwise TMSOTf (23.98 g, 107.87 mmol, 19.49 mL, 1.05 eq), and the mixture was stirred at 60° C. for 4 hours. Upon completion as monitored by LCMS, the mixture was quenched by addition of TEA (60 mL) at 20° C., stirred for 15 min, diluted with DCM (500 mL), and washed with NaHCO₃ (sat., aqueous 300 mL*2). The organic layer was washed with brine (300 mL), dried over Na₂SO₄, filtered, and concentrated under reduced pressure to give compound 40-3 (32.5 g, crude) as a yellow oil. ¹H NMR (400 MHz, CDCl₃) δ=5.96 (d, J=7.3 Hz, 1H), 5.25 (t, J=2.4 Hz, 1H), 4.95-4.88 (m, 1H), 4.19-4.08 (m, 3H), 3.59 (m, 1H), 2.13-2.05 (m, 12H).

Preparation of compound 40-4: To a mixture of compound 40-3 (32.5 g, 98.69 mmol, 1 eq) in DCM (250 mL) was added hex-5-en-1-ol (11.86 g, 118.43 mmol, 13.96 mL, 1.2 eq) and 4A MS (32.5 g). The mixture was stirred at 30° C. for 0.5 h, followed by dropwise addition of TMSOTf (13.16 g, 59.22 mmol, 10.70 mL, 0.6 eq). The mixture was stirred at 30° C. for 16 hours. Upon completion as monitored by LCMS, the reaction mixture was filtered, and the filtrate was diluted with DCM (300 mL) and washed with NaHCO₃ (sat., aqueous 150 mL*2). The organic layer was washed with brine (150 mL), dried over Na₂SO₄, filtered and concentrated under reduced pressure. The residue was purified by flash silica gel chromatography (ISCOR; 220 g SepaFlash® Silica Flash Column, Eluent of 0˜70% PE/EA gradient at 100 mL/min) to give compound 40-4 (12.3 g, 28.64 mmol, 29.02% yield) as a white solid. ¹H NMR (400 MHz, CDCl₃) δ=5.78 (m, 1H), 5.45 (d, J=8.8 Hz, 1H), 5.31 (dd, J=9.4, 10.7 Hz, 1H), 5.06 (t, J=9.5 Hz, 1H), 5.02-4.92 (m, 2H), 4.68 (d, J=8.3 Hz, 1H), 4.30-4.23 (m, 1H), 4.16-4.10 (m, 1H), 3.91-3.76 (m, 2H), 3.73-3.66 (m, 1H), 3.48 (td, J=6.7, 9.5 Hz, 1H), 2.09-2.01 (m, 11H), 1.94 (s, 3H), 1.60-1.36 (m, 4H); LCMS (ESI): m/z calcd. for C₂₀H₃₂NO₉, 430.47 [M+H]⁺, found 430.1.

Preparation of compound 40-5: To a solution of compound 40-4 (12.3 g, 28.64 mmol, 1 eq) in a mixed solvent of DCM (60 mL) and MeCN (60 mL) was added NaIO₄ (2.5 M, 57.28 mL, 5 eq), and the mixture was stirred at 20° C. for 0.5 hours. RuCl₃ (123.00 mg, 592.97 μmol, 0.02 eq) was added, and the mixture was stirred at 20° C. for 2 hours. Upon completion as monitored by LCMS, saturated aqueous NaHCO₃ was added to the mixture to adjust pH>7. The mixture was diluted with DCM (300 mL) and subjected to extraction. The aqueous layer was adjusted to pH<7 by citric acid, and the aqueous layer was extracted with DCM (300 mL*3). The combined organic layers were washed with brine (300 mL), dried over Na₂SO₄, filtered, and concentrated under reduced pressure to give compound 40-5 (8.9 g, 69.31% yield, as a brown solid. ¹H NMR (400 MHz, CDCl₃) δ=6.14 (d, J=8.8 Hz, 1H), 5.34-5.20 (m, 1H), 5.08-5.01 (m, 1H), 4.67 (d, J=8.3 Hz, 1H), 4.24 (dd, J=4.8, 12.3 Hz, 1H), 4.17-4.05 (m, 1H), 3.90-3.83 (m, 2H), 3.75-3.62 (m, 2H), 3.50 (d, J=5.9, 9.9 Hz, 1H), 2.44-2.27 (m, 2H), 2.09-1.93 (m, 12H), 1.75-1.53 (m, 4H); LCMS (ESI): m/z calcd. for C₁₉H₃₀NO₁₁, 448.44 [M+H]+, found 448.1.

Preparation of compound 40-6: To a solution of compound 40-5 (10 g, 22.35 mmol, 1 eq) and 1-hydroxypyrrolidine-2,5-dione (2.83 g, 24.58 mmol, 1.1 eq) in DCM (100 mL) was added EDCI·HCl (5.57 g, 29.05 mmol, 1.3 eq), and the mixture was stirred at 20° C. for 2 hour. Upon completion as monitored by LCMS, the reaction mixture was diluted with DCM (200 mL) and washed with H₂O (100 mL). The organic layer was washed with NaHCO₃ (sat. aqueous) (100 mL*2) and brine (100 mL), dried over Na₂SO₄, filtered, and concentrated under reduced pressure to give compound 40-6 (10.1 g, 82.66%) as a white solid. ¹H NMR (400 MHz, CDCl₃) δ=5.85 (d, J=8.8 Hz, 1H), 5.31-5.26 (m, 1H), 5.06 (t, J=9.7 Hz, 1H), 4.69 (d, J=8.3 Hz, 1H), 4.25 (dd, J=4.7, 12.2 Hz, 1H), 4.12 (dd, J=2.3, 12.2 Hz, 1H), 3.94-3.79 (m, 2H), 3.75-3.65 (m, 1H), 3.63-3.53 (m, 1H), 2.87 (br d. J=4.3 Hz, 4H), 2.76-2.56 (m, 2H), 2.08 (s, 3H), 2.02 (d, J=1.8 Hz, 6H), 1.92 (s, 3H), 1.86-1.66 (m, 4H); LCMS (ESI): m/z calcd. for C₂₃H₃₃N₂O₁₃, 545.51 [M+H]+, found 545.1.

Preparation of compound 40-8: To a solution of compound 40-7 (40-7 prepared by following the general procedure described in WO 2018013999 A1) (9.8 g, 13.92 mmol, 1 eq) in DCM (100 mL) was added DIEA (3.60 g, 27.84 mmol, 4.85 mL, 2 eq), followed by addition of (2,5-dioxopyrrolidin-1-yl) 5-[3-acetamido-4,5-diacetoxy-6-(acetoxymethyl)tetrahydropyran-2-yl]oxypentanoate (compound 40-6) (9.86 g, 18.10 mmol, 1.3 eq), and the mixture was stirred at 20° C. for 2 hours. Upon completion as monitored by LCMS, the reaction mixture was diluted with water (100 mL), and then extracted with DCM (100 mL*2). The combined organic layers were washed brine (100 mL), dried over anhydrous Na₂SO₄, filtered and the filtrate was concentrated under reduced pressure to give a residue. The residue was purified by flash silica gel chromatography (ISCOR; 120 g SepaFlash® Silica Flash Column, Eluent of 0˜6% MeOH/DCM gradient at 80 mL/min) to give compound 40-8 (13.1 g, 80.95% yield,) as a white solid. ¹H NMR (400 MHz, DMSO-d6) δ=8.06 (d, J=9.3 Hz, 1H), 7.81 (q, J=5.4 Hz, 2H), 7.21 (d, J=8.8 Hz, 6H), 6.84 (d, J=9.0 Hz, 6H), 5.04 (t, J=10.0 Hz, 1H), 4.78 (t, J=9.7 Hz, 1H), 4.55 (d, J=8.5 Hz, 1H), 4.17 (dd, J=4.5, 12.3 Hz, 1H), 3.97 (d, J=10.0 Hz, 1H), 3.77 (dd, J=2.6, 9.9 Hz, 1H), 3.72-3.64 (m, 11H), 3.46-3.25 (m, 5H), 3.05-2.84 (m, 8H), 2.18 (t, J=7.2 Hz, 2H), 2.05-1.95 (m, 7H), 1.93 (s, 3H), 1.88 (s, 3H), 1.74 (s, 3H), 1.47-1.13 (m, 20H); LCMS (ESI): RT=2.017 min, m/z calcd. for C₆₀H₈₄NaN₄O₁₇, 1156.32 [M+Na]⁺, 1155.5.

Preparation of compound 40-9: To a mixture of compound 40-8 (5 g, 4.41 mmol, 1 eq) and 4A MS (5 g) in DCM (50 mL) was added 3-bis(diisopropylamino)phosphanyloxypropanenitrile (1.73 g, 5.74 mmol, 1.82 mL, 1.3 eq) at −10° C., followed by addition of 1H-imidazole-4,5-dicarbonitrile (573.12 mg, 4.85 mmol, 1.1 eq), and the mixture was stirred at 0° C. for 2 hours. Upon completion as monitored by LCMS, the reaction mixture was diluted with DCM (100 mL), washed with NaHCO₃ (sat., aqueous, 50 mL*2), dried over Na₂SO₄, and concentrated under reduced pressure to give a pale yellow foam. The residue was purified by flash silica gel chromatography (ISCO®; 40 g SepaFlash® Silica Flash Column, 0% to 10% i-PrOH in DCM contain 2% TEA) to give compound 40-9 (3.35 g, 56.60% yield,) as a white solid. ¹H NMR (400 MHz, CD₃CN) δ=7.35-7.25 (m, 6H), 6.88-6.82 (m, 6H), 6.79 (d, J=9.3 Hz, 1H), 6.63-6.46 (m, 2H), 5.17-5.08 (m, 1H), 4.93 (t, J=9.7 Hz, 1H), 4.59 (d, J=8.6 Hz, 1H), 4.22 (dd, J=4.9, 12.2 Hz, 1H), 4.04 (dd, J=2.4, 12.2 Hz, 1H), 3.85-3.32 (m, 22H), 3.15-3.00 (m, 8H), 2.59 (t, J=5.8 Hz, 2H), 2.23 (br t, J=6.6 Hz, 3H), 2.12-2.04 (m, 4H), 2.00 (s, 3H), 1.96 (s, 3H), 1.93 (s, 3H), 1.82 (s, 3H), 1.66-1.45 (m, 12H), 1.42-1.21 (m, 6H), 1.19-1.07 (m, 12H); LCMS (ESI) m/z calcd. for C₆₉H₁₀₁NaN₆O₁₈P 1355.68 [M+Na]⁺, found 1355.7; ³¹P NMR (CD₃CN) δ=147.00.

### Example 19: Preparation of GalNAc4 CPG

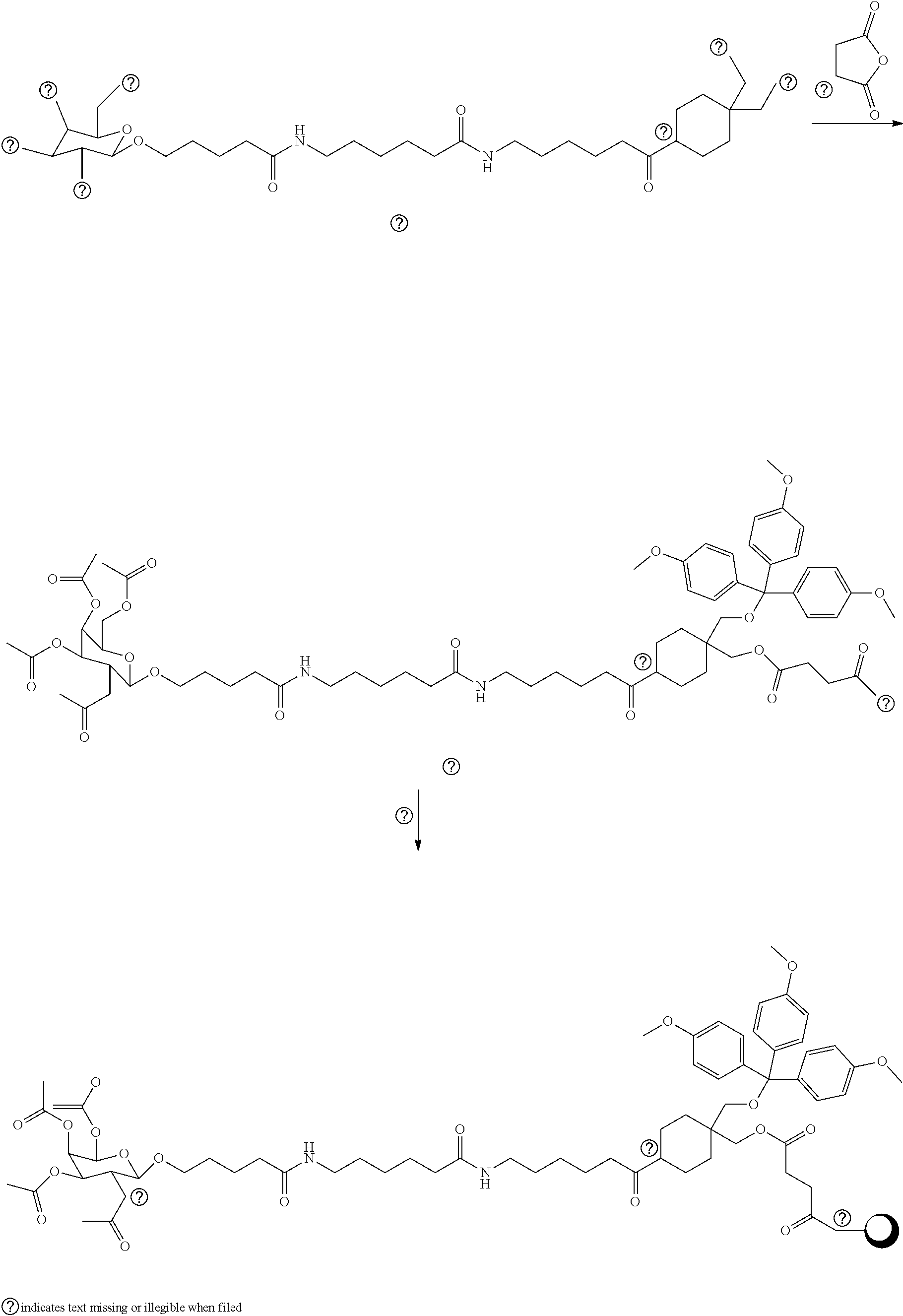

To a solution of 1 (21 g, 18.53 mmol, 1 eq) and succinic anhydride (9.27 g, 92.65 mmol, 5 eq) in DCM (160 mL) were added TEA (18.75 g, 185.30 mmol, 25.79 mL, 10 eq) and DMAP (2.26 g, 18.53 mmol, 1 eq) at 15° C. The mixture was stirred at 15° C. for 16 h. TLC (DCM:MeOH=10:1) showed the reaction was complete. The reaction mixture was diluted with water (200 mL), and then extracted with DCM (300 mL*2). The combined organic layers were washed with brine (300 mL*3), dried over anhydrous Na₂SO₄, concentrated under reduced pressure. The residue was purified by flash silica gel chromatography (ISCOR; 220 g SepaFlash® Silica Flash Column, Eluent of 0˜10% MeOH/DCM/TEA DCM was added 0.5 @ 100 mL/min) to give AGS-6-5 (12.8 g, 56% yield) LCMS: (ESI): m/z 1233.6 [M+H]⁺. Further Succinate AGS-6-5 was loaded onto LCAA (CNA) 500 Å CPG by following the general procedure to give GalNAc 4 CPG.

### Example 20—Preparation of Compound 269

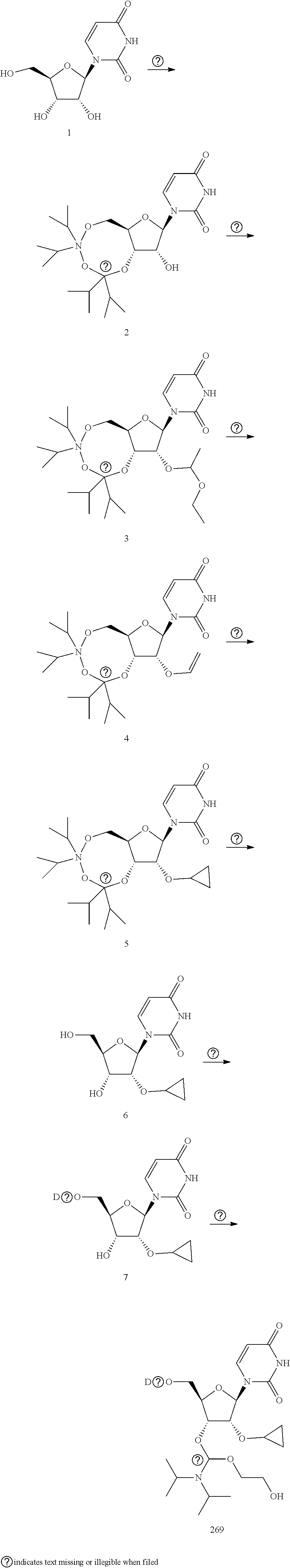

Preparation of compound 2: To a solution of 1 (100.0 g, 409.8 mmol) in DMF (1.0 L). Then the mixture was added imidazole (69.7 g, 1024.5 mmol), TiPSCl₂ (142.2 g, 450.8 mmol) under N₂ atmosphere. The mixture was stirred at r.t for 4 h. LC-MS and TLC show SM was completely consumed. Then the solution was diluted with EA, washed with water twice. Then the combined organic layers were washed the organic phase once with saturated brine and dried over by Na₂SO₄. Then the solution was concentrated under reduced pressure. This resulted was purified by column chromatograph (eluent, PE/EA=100:1˜1:1). This resulted in 2 (185.0 g, 380.5 mmol, 92.8% yield) as a white solid. ESI-LCMS: m/z 487.5 [M+H]⁺, ¹H NMR (400 MHz, DMSO-d₆) δ: 11.4-11.3 (m, 1H), 7.72-7.62 (m, 1H), 5.63-5.47 (m, 3H), 4.20-3.86 (m, 5H), 1.12-0.92 (m, 28H).

Preparation of compound 3: To a stirred solution of 2 (185.0 g, 380.5 mmol) and ethyl vinyl ether (356.1 g, 4946.5 mmol) in DCM (1.8 L) was added PPTS (19.1 g, 76.1 mmol). The resulting mixture was stirred at r.t under argon atmosphere for overnight. LC-MS and TLC show SM was completely consumed. The reaction was quenched by the addition of aq. NH₄OAc (200 mL) at r.t. The resulting mixture was extracted with EA. Then the combined organic layers were washed the organic phase once with saturated brine and dried over by Na₂SO₄. Then the solution was concentrated under reduced pressure. This resulted in crude 3 (230.0 g) as a yellow solid without further purified and used directly for the next step. ESI-LCMS: m/z 559.2 [M+H]⁺.

Preparation of compound 4: To the solution of 3 (230.0 g, crude) in DCM (2.3 L) was treated with TEA (499.6 g, 4946.5 mmol) under nitrogen atmosphere followed by the addition of TMSOTf (845.7 g, 3805.0 mmol) drop wise at 0° C. The resulting mixture was stirred at r.t under argon atmosphere for overnight. LC-MS and TLC show SM was completely consumed. The reaction was quenched with sat. NH₄OAc (aq.) at 0° C. The resulting mixture was extracted with EA. Then the combined organic layers were washed the organic phase once with saturated brine and dried over by Na₂SO₄. Then the solution was concentrated under reduced pressure. This resulted was purified by silica gel column chromatograph (eluent, PE/EA=100:1˜1:1). This resulted in 4 (142.0 g, 277.3 mmol) as a white solid. ESI-LCMS: m/z 511.2 [M−H]⁺.

Preparation of compound 5: To a stirred solution 4 (70.0 g, 136.7 mmol) and DIM (146.4 g, 546.8 mmol) in DCM (1.0 L) was added 1M. ZnEt₂ (547 mL, 546.8 mmol) drop wise 1 h at r.t under argon atmosphere. LC-MS and TLC show SM was completely consumed. The reaction was quenched with sat. NH₄OAc (aq.) at r.t. The resulting mixture was extracted with EA. Then the combined organic layers were washed the organic phase once with saturated brine and dried over by Na₂SO₄. Then the solution was concentrated under reduced pressure. This resulted was purified by silica gel column chromatograph (eluent, PE/EA=100:1˜1:1). This resulted in 5 (25.0 g, 47.5 mmol, 34.7% yield) as a white solid. ESI-LCMS: m/z 526.3 [M+H]⁺.

Preparation of compound 6: To a solution of 5 (25.0 g, 47.5 mmol) in THF (250 mL) and was added IM TBAF (71 ml, 71.2 mmol). The mixture was stirred at r.t. for 3 h. LC-MS showed 5 was consumed completely. H₂O was added to the mixture. Then the solution diluted with EA. The organic layer was washed with NaHCO₃ (aq.) and brine and dried over by Na₂SO₄. Then the solution was concentrated under reduced pressure. This resulted was purified by silica gel column chromatograph (eluent, DCM/MeOH=100:1˜30:1). This resulted in 6 (11.5 g, 40.3 mmol, 84.9% yield) as a white solid. ESI-LCMS: m/z 285.2 [M+H]⁺; ¹H NMR (400 MHz, DMSO-d₆) δ: 7.95 (s, 1H), 8.05-7.85 (m, 1H), 5.95-5.55 (m, 2H), 5.25-5.10 (m, 2H), 4.20-3.40 (m, 6H), 0.65-0.30 (m, 4H).

Preparation of compound 7: To a stirred mixture of 6 (11.5 g, 40.3 mmol) in pyridine (110 mL) was added DMTrCl (16.4 g, 48.3 mmol) at r.t under N₂ atmosphere. The resulting mixture was stirred at r.t under argon atmosphere for 3 h. LC-MS and TLC show SM was completely consumed. The reaction was quenched by the addition of sat. NaHCO₃(aq.). The resulting mixture was extracted with EA. Then the combined organic layers were washed the organic phase once with saturated brine and dried over by Na₂SO₄. Then the solution was concentrated under reduced pressure. This resulted was purified by silica gel column chromatograph (eluent, PE (0.5% TEA)/EA=50:1˜2:3). This resulted in 7 (22.0 g, 37.4 mmol, 92.8% yield) as a white solid. ESI-LCMS: m/z 585.1 [M−H]⁺; ESI-LCMS: m/z 585.1 [M−H]⁺. ¹H NMR (400 MHz, DMSO-d₆) δ: 11.39 (d, J=1.9 Hz, 1H), 7.72 (d, J=8.1 Hz, 1H), 7.45-7.17 (m, 9H), 7.00-6.79 (m, 4H), 5.82 (d, J=4.2 Hz, 1H), 5.46-4.95 (m, 2H), 4.24 (q, J=5.7 Hz, 1H), 4.07 (t, J=4.8 Hz, 1H), 3.94 (dd, J=9.6, 5.6 Hz, 1H), 3.74 (s, 6H), 3.58 (tt, J=6.0, 3.0 Hz, 1H), 3.30-3.15 (m, 2H), 0.71-0.31 (m, 4H).

Preparation of compound 269: To a solution of 7 (5.5 g, 9.4 mmol) in DCM (55 mL) was added DCI (943 mg, 8.0 mmol) and CEP[N(iPr)₂]₂ (3.4 g, 11.3 mmol) under N₂. The mixture was stirred at r.t for 2.5 h. LC-MS showed 7 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NH₄HCO₃)=1/3 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 269 (5.1 g, 6.4 mmol, 92.8% yield, 98.0% purity) as a white solid. ESI-LCMS: m/z 787.3 [M+H]⁺; ¹H NMR (400 MHz, DMSO-d₆) δ: 11.39 (s, 1H), 7.80 (dd, J=13.8, 8.1 Hz, 1H), 7.44-7.20 (m, 9H), 6.90 (ddd, J=8.8, 6.2, 2.6 Hz, 4H), 5.83 (dd, J=5.9, 3.9 Hz, 1H), 5.28 (dd, J=21.8, 8.1 Hz, 1H), 4.45 (ddt, J=30.2, 10.8, 5.5 Hz, 1H), 4.29-4.19 (m, 1H), 4.08 (ddt, J=17.4, 6.2, 3.3 Hz, 1H), 3.89-3.44 (m, 12H), 3.34 (d, J=3.3 Hz, 1H), 3.31-3.26 (m, 1H), 2.78 (t, J=5.9 Hz, 1H), 2.63 (dt, J=6.9, 5.0 Hz, 1H), 1.26-0.92 (m, 12H), 0.66-0.42 (m, 4H). ³¹P NMR (162 MHz, DMSO-d₆) δ: 149.39, 148.28.

### Example 21—Preparation of Compound 272

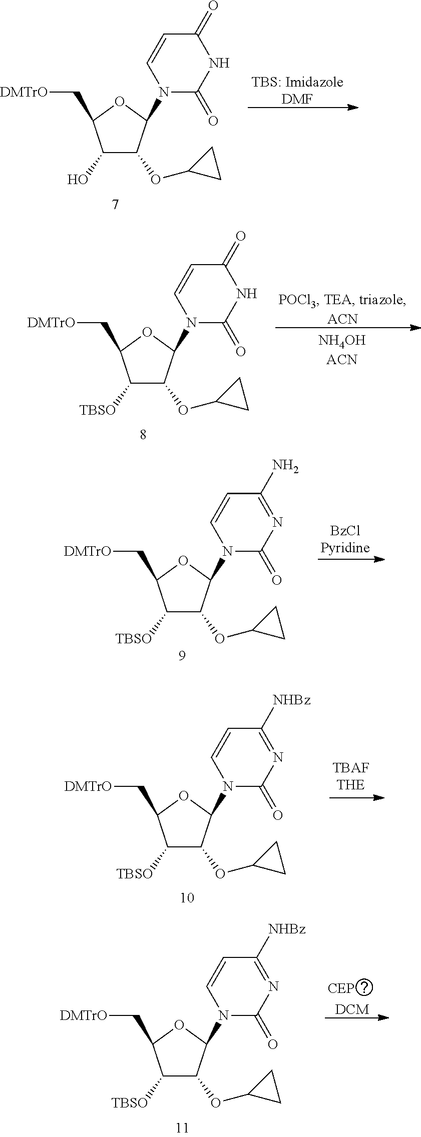

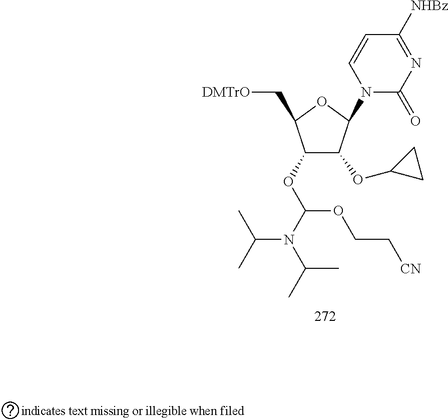

Preparation of compound 8: To a solution of 7 (9.8 g, 16.7 mmol) in DMF (950 mL) was added Imidazole (3.6 g, 53.5 mmol) and TBSCl (3.0 g, 20.0 mmol) at 0° C. under N₂ atmosphere. The mixture was stirred at r.t at N₂ for overnight. LC-MS and TLC show SM was completely consumed. Then the solution was diluted with EA, washed with water twice. Then the combined organic layers were washed the organic phase once with saturated brine and dried over by Na₂SO₄. Then the solution was concentrated under reduced pressure. This resulted in crude 2 (12.2 g) as a white solid without further purified and used directly for the next step. ESI-LCMS: m/z 699.4 [M−H]⁺.

Preparation of compound 9; To a solution of 8 (12.1 g, 17.1 mmol) in ACN (120 mL) was added triazole (9.4 g, 136.2 mmol) and TEA (27.6 g, 273.2 mmol) at r.t. Then the mixture was added POCl₃ (5.2 g, 33.9 mmol) drop wise at 0° C. at N₂. The mixture was stirred at r.t for 5 h. LC-MS and TLC show SM was completely consumed. Then the mixture was added NH₄OH (60 mL) at r.t. The mixture was stirred at r.t. for 15 h. LC-MS and TLC show SM was completely consumed. Then the solution was diluted with EA, washed with water twice. Then the combined organic layers were washed the organic phase once with saturated brine and dried over by Na₂SO₄. Then the solution was concentrated under reduced pressure. This resulted in crude 9 (13.6 g) as a white solid without further purified and used directly for the next step. ESI-LCMS: m/z 700.2 [M+H]⁺.

Preparation of compound 10: To a stirred solution of 9 (11.0 g, 15.7 mmol) in Pyridine (110 mL) was added BzCl (2.6 g, 18.4 mmol) drop wise at 0° C. The resulting mixture was stirred at r.t at N₂ for 3 h. LC-MS and TLC show SM was completely consumed. Then the solution was diluted with EA, washed with water twice. Then the combined organic layers were washed the organic phase once with saturated brine and dried over by Na₂SO₄. Then the solution was concentrated under reduced pressure. This resulted in crude 10 (13.0 g) as a yellow solid without further purified and used directly for the next step. ESI-LCMS: m/z 804.2 [M+H]⁺.

Preparation of compound 11: To a solution of 10 (13.0 g, 16.1 mmol) in THE (130 mL) was added 1 M TBAF (32 mL, 32.3 mmol) at r.t. The mixture was stirred at r.t. for 15 h. LC-MS and TLC show SM was completely consumed. Then the solution was diluted with EA, washed with water twice. Then the combined organic layers were washed the organic phase once with saturated brine and dried over by Na₂SO₄. Then the solution was concentrated under reduced pressure. This resulted was purified by column chromatograph (eluent, PE/EA=5:1˜1:1) to give a crude. The crude was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=4/1; Detector, UV 254 nm. This resulted in 11 (5.6 g, 8.1 mmol, 48.5% yield over 4 steps, 98.0% purity) as a white solid. ESI-LCMS: m/z 487.5 [M+H]⁺. ¹H NMR (400 MHz, DMSO-d₆) δ: 11.30 (s, 1H), 8.37 (d, J=7.6 Hz, 1H), 8.00 (d, J=7.7 Hz, 2H), 7.63 (t, J=7.2 Hz, 1H), 7.52 (t, J=7.6 Hz, 2H), 7.46-7.24 (m, 9H), 7.16 (d, J=7.5 Hz, 1H), 6.93 (d, J=8.4 Hz, 4H), 5.87 (s, 1H), 5.24 (d, J=7.0 Hz, 1H), 4.33 (d, J=6.3 Hz, 1H), 4.03 (d, J=6.9 Hz, 2H), 3.76 (s, 6H), 1.99 (s, 1H), 1.24 (s, 1H), 1.17 (t, J=7.1 Hz, 1H), 0.68 (s, 1H), 0.61-0.52 (m, 1H), 0.48 (d, J=6.1 Hz, 2H).

Preparation of compound 272: To a solution of 11 (4.9 g, 7.1 mmol) in DCM (50 mL) was added DCI (712 mg, 6.0 mmol) and CEP[N(iPr)₂]₂ (2.6 g, 8.5 mmol) at r.t at N₂. The mixture was stirred at r.t for 2 h. LC-MS showed 11 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 272 (4.9 g, 5.5 mmol, 77% yield, 98.0% purity) as a white solid. ESI-LCMS: m/z 787.3 [M+H]⁺; ¹H NMR (400 MHz, DMSO-d₆) δ: 11.26 (s, 1H), 8.59-8.33 (m, 1H), 8.06-7.93 (m, 1H), 7.67-7.22 (m, 12H), 7.17-7.06 (m, 1H), 6.98-6.85 (m, 1H), 6.00-5.89 (m, 1H), 4.63-4.41 (m, 1H), 4.28-4.09 (m, 2H), 3.86-3.35 (m, 13H), 2.80-2.56 (m, 2H), 1.20-0.91 (m, 12H), 0.70-0.41 (m, 4H).

### Example 22—Preparation of Compound 270

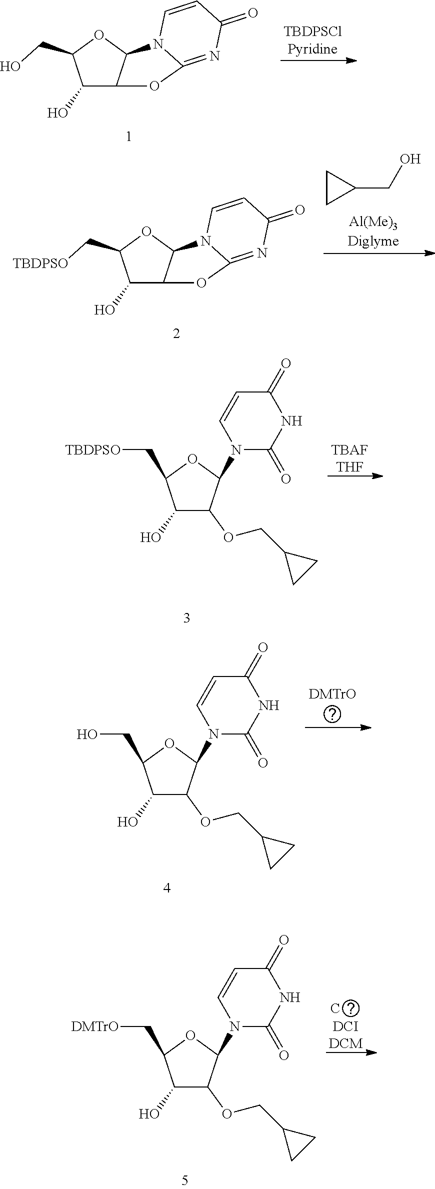

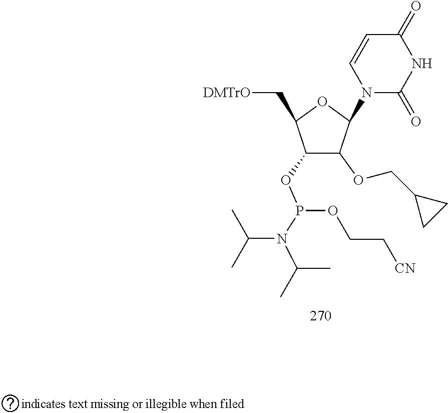

Preparation of compound 2: To a solution of compound 1 (50 g, 221.2 mmol) in pyridine (500 mL) was added TBDPSCl (86.2 g, 265.2 mmol). The mixture was stirred for 16 h until compound 1 was consumed, detected by TLC. The reaction was quenched with H₂O and extracted by EA. The organic layer was washed with water, brine, dried over anhydrous sodium sulfate and evaporated in vacuo. The resulting crude residue was purified by column chromatography (SiO₂, PE:EA=1:1) to give compound 2 (93 g, 93% yield, 98% purity) as a white solid. ESI-MS: m/z 465.2 [M+H]⁺

Preparation of compound 3: To a solution of cyclopropyl carbinol (245.3 g, 3.4 mol) in diglyme (300 mL) then cooled in ice bath, Al(Me)₃ (440.4 mmol, 220 mL) was slowly dropped in mixture over 1 h, then the mixture was heated to 100° C. for 1 h, compound 2 (93 g, 200.1 mmol) was added. The resulting mixture was stirred for 16 h at 120° C., TLC showed 2 was consumed completely. The mixture was slowly added in 10% H₃PO₄ until the solid was dissolved completely, then extracted with EA, washed with NaHCO₃ aqueous solution and brine, dried over anhydrous Na₂SO₄ and filtered. The filtrate was concentrated under reduced pressure to give a residue. The crude was purified by column chromatography (SiO₂, 5% MeOH in DCM) to give 3 (55 g, 51% yield, 98% purity) as a white solid. ¹H NMR (400 MHz, DMSO_{d6}) δ 11.19 (s, 1H), 7.53 (d, J=8.1 Hz, 1H), 7.47-7.42 (m, 4H), 7.32-7.23 (m, 6H), 5.66 (d, J=4.1 Hz, 1H), 5.06 (dd, J=8.0, 2.0 Hz, 1H), 4.98 (d, J=6.4 Hz, 1H), 4.01 (m, 1H), 3.75 (m, 3H), 3.61 (m, 1H), 3.21 (m, 2H), 0.84 (m, 10H), 0.25 (m, 2H), 0.00 (m, 2H). ESI-MS: m/z 537.2 [M+H]⁺

Preparation of compound 4: To a solution of 3 (55 g, 102.5 mmol) in THE (500 mL) was added TBAF (123 mmol, 123 mL), Then the mixture was stirred for 1 h at 20° C., TLC showed 3 was consumed completely. The mixture was added H₂O, extracted with EA, washed with H₂O twice and brine, dried over anhydrous Na₂SO₄ and filtered. The filtrate was concentrated under reduced pressure to give a residue. The crude was filtered by column chromatography (SiO₂, 5% MeOH in EA) to give 4 (21 g, 65% yield, 98% purity) as a white solid. ¹H NMR (400 MHz, DMSO_{d6}) δ 11.33 (s, 1H), 7.93 (d, J=8.1 Hz, 1H), 5.84 (d, J=5.1 Hz, 1H), 5.64 (d, J=8.0 Hz, 1H), 5.12 (t, J=5.0 Hz, 1H), 5.05 (d, J=5.8 Hz, 1H), 4.07 (m, 1H), 3.93 (m, 3H), 3.85 (m, 1H), 3.65-3.52 (m, 2H), 3.35 (m, 2H), 0.98 (m, 1H), 0.44 (m, 2H), 0.17 (m, 2H). ESI-MS: m/z 299 [M+H]⁺

Preparation of compound 5: To a solution of 4 (21 g, 70.4 mmol) in pyr (200 mL) was added DMTrCl (26.1 g, 77.4 mmol). The resulting mixture was stirred for 16 h at 20° C., TLC showed 4 was consumed completely. The reaction was added NaHCO₃ solution, then extracted with EA, dried over anhydrous Na₂SO₄ and filtered. The filtrate was concentrated under reduced pressure to give a residue. The crude was filtered by column chromatography (SiO₂, 10% MeOH in DCM) to give 5 (38 g, 90% yield, 98% purity) as a white solid. ¹H NMR (400 MHz, DMSO_{d6}) δ 11.18 (s, 1H), 7.54 (d, J=8.1 Hz, 1H), 7.19-7.05 (m, 9H), 6.72 (m, 4H), 5.61 (d, J=3.2 Hz, 1H), 5.10 (d, J=8.0 Hz, 1H), 4.94 (d, J=6.6 Hz, 1H), 3.98 (m, 1H), 3.77 (m, 2H), 3.55 (m, 6H), 3.23 (m, 2H), 3.08 (m, 2H), 0.84 (m, 1H), 0.26 (m, 2H), 0.03 (m, 2H). ESI-MS: m/z 601.2 [M+H]⁺.

Preparation of compound 270: To a solution of 5 (11 g, 18.3 mmol) in DCM (100 mL) was added DCI (1.8 g, 15.6 mmol), CEP[N(iPr)₂]₂ (6.6 g, 21.9 mmol). The resulting mixture was stirred for 1 h at 20° C., TLC showed 5 was consumed completely. The reaction was added NaHCO₃ solution, then extracted with DCM, dried over anhydrous Na₂SO₄ and filtered. The filtrate was concentrated under reduced pressure to give a residue. The crude was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in 270 (11.5 g, 80% yield, 98% purity) as a white solid. ¹H NMR (400 MHz, DMSO_{d6}) δ 11.37 (s, 1H), 7.80 (m, 1H), 7.41-7.23 (m, 9H), 6.89 (m, 4H), 5.80 (m, 1H), 5.31 (dd, J=22.8, 8.0 Hz, 1H), 4.45-4.31 (m, 1H), 4.11 (m, 2H), 3.86-3.26 (m, 14H), 2.77-2.56 (m, 2H), 1.17-0.95 (m, 13H), 0.45 (m, 2H), 0.17 (m, 2H); ³¹P NMR (162 MHz, DMSO-_{d6}) δ 149.03, 148.60. ESI-LCMS: m/z 801.4 [M+H]⁺.

### Example 23—Preparation of Compound 273

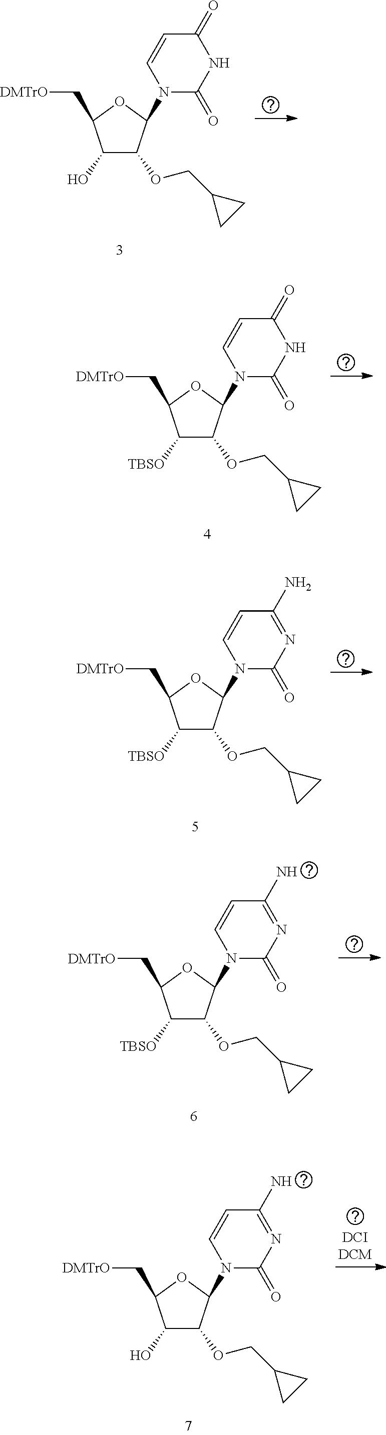

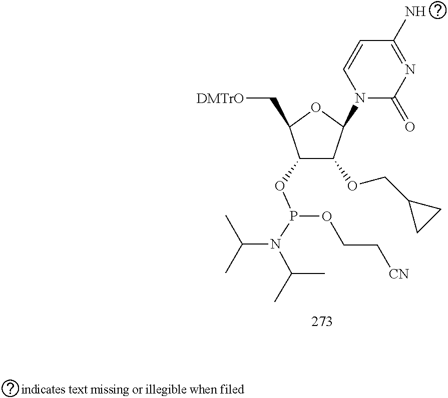

Preparation of compound 7: To a solution of 5 (26 g, 43.3 mmol) in DCM (280 mL) was added imidazole (7.3 g, 108.2 mmol), TBSCl (5.3 g, 64.9 mmol). The resulting mixture was stirred for 16 h at 20° C., TLC showed 5 was consumed completely. The reaction was added NaHCO₃ solution, then extracted with EA, dried over anhydrous Na₂SO₄ and filtered. The filtrate was concentrated under reduced pressure to give a residue. The crude was purified by column chromatography (SiO₂, PE:EA=1:1) to give 7 (27 g, 85% yield, 98% purity) as a white solid. ¹H NMR (400 MHz, DMSO_{d6}) δ 11.43 (s, 1H), 7.91 (d, J=8.1 Hz, 1H), 7.44-7.27 (m, 9H), 6.96 (m, 4H), 5.84 (d, J=2.6 Hz, 1H), 5.34 (m, 1H), 4.34 (m, 1H), 4.01 (m, 2H), 3.79 (m, 6H), 3.54 (m, 1H), 3.35 (m, 1H), 3.26 (m, 1H), 1.04 (m, 1H), 0.81 (s, 9H), 0.50 (m, 2H), 0.22 (m, 2H), 0.08 (s, 3H), 0.00 (s, 3H). ESI-MS: m/z 715.4 [M+H]⁺.

Preparation of compound 8: To a solution of 7 (27 g, 37.8 mmol) in ACN (270 mL) was added triazole (20.8 g, 302 mmol), TEA (61.1 g, 604 mmol), then cooled down to 0° C., POCl₃ (11.5 g, 75.5 mmol) was dropped. The resulting mixture was stirred for 40 min at 0° C., TLC showed 7 was consumed completely. NH₃·H₂O (140 ml) was added. The mixture was stirred for 16 h at 20° C. Then the reaction was added NaHCO₃ solution, then extracted with EA, washed with H₂O and brine, dried over anhydrous Na₂SO₄ and filtered. The filtrate was concentrated under reduced pressure to give a crude 8 (30 g) as a white solid. ¹H NMR (400 MHz, DMSO_{d6}) δ 7.95 (m, 1H), 7.47-7.11 (m, 11H), 6.88 (m, 4H), 5.83 (s, 1H), 5.53 (m, 1H), 4.28 (m, 1H), 3.96 (m, 2H), 3.86-3.55 (m, 5H), 3.45-3.33 (m, 2H), 3.16 (m, 1H), 1.15 (m, 1H), 0.99 (m, 1H), 0.72 (s, 9H), 0.43 (m, 2H), 0.16 (m, 2H), 0.00 (s, 3H), −0.10 (s, 3H). ESI-MS: m/z 714.4 [M+H]⁺.

Preparation of compound 9: To a solution of 8 (30 g, 42 mmol) in DCM (300 mL) was added pyr (37.4 g, 420 mmol), then cooled down to 0° C., BzCl (7.1 g, 50.4 mmol) was dropped. The resulting mixture was stirred for 6 h at 25° C., TLC showed 8 was consumed completely. The reaction was added NaHCO₃ solution, then extracted with DCM, dried over anhydrous Na₂SO₄ and filtered. The filtrate was concentrated under reduced pressure to give a residue. The crude was purified by column chromatography (SiO₂, PE:EA=3:1) to give 9 (24.5 g, 80% yield, 98% purity) as a white solid. ¹H NMR (400 MHz, DMSO_{d6}) δ 11.27 (s, 1H), 8.54 (d, J=7.2 Hz, 1H), 7.96 (m, 2H), 7.61-7.12 (m, 13H), 6.90 (m, 4H), 5.85 (s, 1H), 4.38 (m, 1H), 4.03 (m, 1H), 3.90 (d, J=4.9 Hz, 1H), 3.75-3.68 (m, 7H), 3.52 (m, 1H), 3.35 (m, 1H), 3.23 (m, 1H), 1.01 (m, 1H), 0.70 (s, 9H), 0.45 (m, 2H), 0.18 (m, 2H), 0.00 (s, 3H), −0.11 (s, 3H). ESI-MS: m/z 818.2 [M+H]⁺.

Preparation of compound 10: To a solution of 9 (24.5 g, 29.9 mmol) in THE (240 mL) was added TBAF (32.9 mmol, 33 mL), then the mixture was stirred for 1 h at 20° C., TLC showed 9 was consumed completely. The mixture was added H₂O, extracted with EA, washed with H₂O twice and brine, dried over anhydrous Na₂SO₄ and filtered. The filtrate was concentrated under reduced pressure to give a residue. The crude was purified by Flash-Prep-HPLC with the following conditions (IntelFlash-1): Column, Cis silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm. This resulted in 10 (18.5 g, 90% yield, 98% purity) as a white solid. ¹H NMR (400 MHz, DMSO_{d6}) δ 11.28 (s, 1H), 8.40 (d, J=7.2 Hz, 1H), 7.99 (m, 2H), 7.64-7.12 (m, 13H), 6.90 (m, 4H), 5.85 (m, 1H), 5.12 (d, J=7.2 Hz, 1H), 4.28 (m, 1H), 4.07 (m, 1H), 3.92 (m, 1H), 3.76 (m, 6H), 3.58 (m, 2H), 3.36 (m, 2H), 2.29 (s, 3H), 1.08 (m, 1H), 0.46 (m, 2H), 0.22 (m, 2H). ESI-MS: m/z 704.2 [M+H]⁺.

Preparation of compound 273: To a solution of 10 (11 g, 15.6 mmol) in DCM (110 mL) was added DCI (1.6 g, 13.3 mmol), CEP[N(iPr)₂]₂ (5.6 g, 18.7 mmol). The resulting mixture was stirred for 1 h at 20° C., TLC showed 10 was consumed completely. The reaction was added NaHCO₃ solution, then extracted with DCM, dried over anhydrous Na₂SO₄ and filtered. The filtrate was concentrated under reduced pressure to give a residue. The crude was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 273 (12.2 g, 90% yield, 99% purity) as a white solid. ¹H NMR (400 MHz, DMSO_{d6}) § 11.26 (s, 1H), 8.46 (dd, J=7.2, 24.0 Hz, 1H), 7.99 (m, 2H), 7.64-7.12 (m, 13H), 6.91 (m, 4H), 5.89 (m, 1H), 4.50 (m, 1H), 4.21 (m, 1H), 4.11 (m, 1H), 3.85-3.43 (m, 13H), 3.37 (m, 1H), 2.75 (t, J=6.0 Hz, 1H), 2.60 (m, 1H), 1.18-0.95 (m, 13H), 0.48 (m, 2H), 0.23 (m, 2H); ³¹P NMR (162 MHz, DMSO-_{d6}) δ 149.26, 148.35 ESI-LCMS m/z 904.2 [M+H]⁺.

### Example 24—Preparation of Compounds 274 & 275

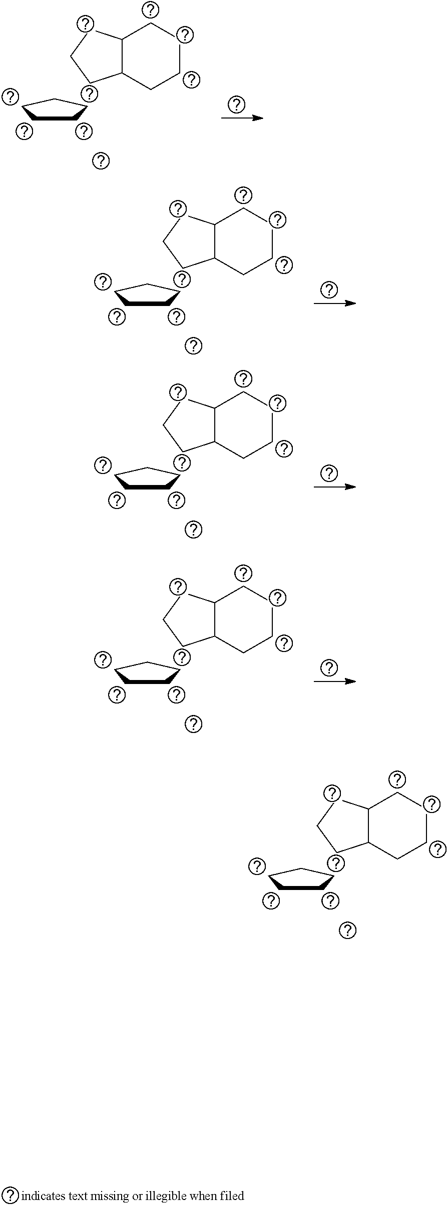

### Preparation of Intermediate 5

Preparation of 2: To a solution of 1 (80 g, 269.1 mmol) in DMF (640 mL), imidazole (18.3 g, 269.1 mmol) and TBSCl (22.16 g, 269.12 mmol) was added at r.t. The solution was stirred 16 hours at r.t. 1 was consumed by LCMS. The solution was added DCM (2000 mL), washed with water (3×3000 mL). The organic phase dried over anhydrous Na₂SO₄, concentrated by rotary evaporator to give 2 (140 g, 252.9 mmol, 93.9% yield) as a white solid. ESI-LCMS m/z 526.3 [M+H]⁺

Preparation of 3: To a solution of 2 (137 g, 260.5 mmol) in pyridine (1000 mL), iBuCl (41.6 g, 390.8 mmol) was added at 0° C. slowly. The solution was stirred 2 hours at r.t. 2 was consumed by LCMS. The solution was added EA (2000 mL), washed with water (3×5000 mL). The organic phase dried over anhydrous Na₂SO₄, concentrated by rotary evaporator to give 3 (155 g, 247.1 mmol, 94.8% yield) as a white solid. ESI-LCMS m/z 596.3 [M+H]⁺

Preparation of 4: To a solution of 3 (145 g, 243.3 mmol) in THF (725 mL) added TFA:H₂O=1:1 (290 mL) was stirred 2 h at r.t. 3 was consumed by LCMS. Con. NH₄OH was added to the mixture to give the pH=7 at 0° C. EA (2000 mL) was added to the mixture. The combined organic layer was washed with water (3×3000 mL). The organic phase dried over anhydrous Na₂SO₄, concentrated by rotary evaporator to give crude. The crude was purified by silica gel; mobile phase, DCM/MeOH=100/1 increasing to DCM/MeOH=20/1, the eluted product was collected at DCM/MeOH=30/1 to give 4 (100 g, 201.4 mmol, 82.7% yield) as a white solid. ESI-LCMS m/z 482.2 [M+H]⁺

Preparation of compound 5: To a solution of 4 (50 g, 103.8 mmol) in DMSO (500 mL), EDCI (59.7 g, 311.4 mmol) was added at 0° C. in N₂. Then pyridine (9.03 g, 114.20 mmol) and TFA (5.92 g, 51.91 mmol) was added to the mixture at 0° C. The solution was stirred 2 h at r.t. The reaction was worked well by LCMS. The reaction was diluted with BA (500 mL). The organic layer was washed with water (3×1000 mL). The organic phase dried over anhydrous Na₂SO₄, concentrated by rotary evaporator to give 5 (48 g, 95.1 mmol, 91.6% yield) as a white solid. ESI-LCMS m/z 480.3 [M+H]⁺

### Preparation of Compound 274

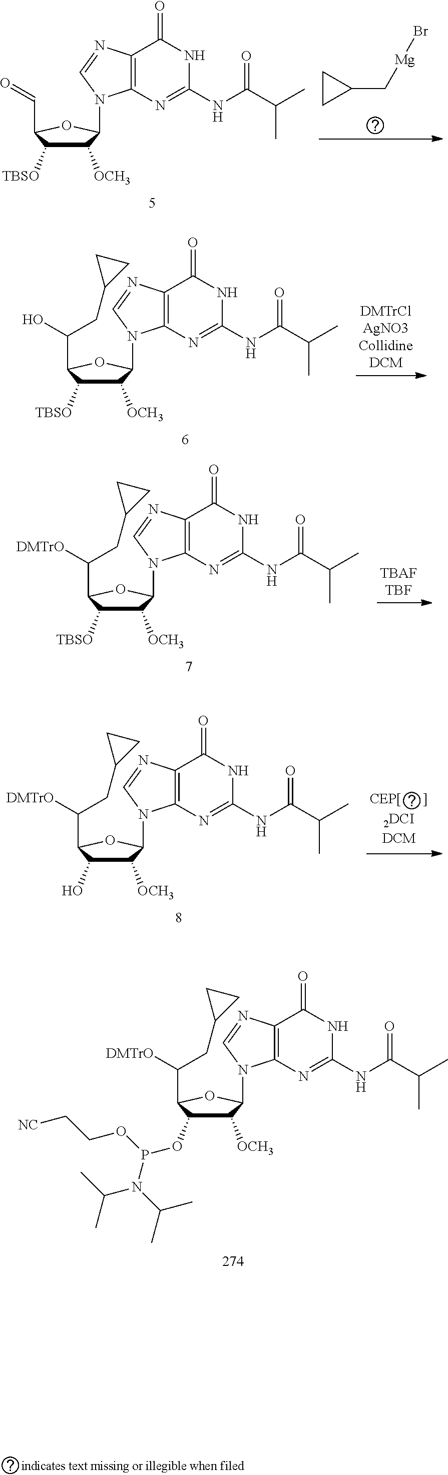

Preparation of compound 6: To a solution of 5 (13.0 g, 27.1 mmol) in THF (20 mL). Then the mixture was drop dawn into (Cyclopropylmethyl) magnesium Bromide (1M, 120 mL) under N₂ atmosphere at 0° C. The mixture was stirred at r.t. for 6 h. Then the solution was diluted with EA, washed with NH₄Cl (aq.) twice. The solvent was concentrated under reduced pressure to give the crude. The crude was purified by Flash-Prep-HPLC with the following conditions (IntelFlash-1): Column, C18 silica gel; mobile phase, CH₃CN/H₂O (0.5% NHAHCO₃)=1/1 increasing to CH₃CN/H₂O (0.5% NHHCO₃)=1/0 within 25 min, the eluted product was collected at CH₃CN/H₂O (0.5% NH₄HCO₃)=2/1; Detector, UV 254 nm, filtered to get 6 (3.7 g, 6.91 mmol, 25.50% yield) as a solid. ESI-LCMS: m/z 536 [M+H]⁺. ¹H NMR (600 MHz, DMSO-d₆) δ 11.84 (d, J=8.4 Hz, 1H), 11.34 (d, J=3.7 Hz, 1H), 8.06 (d, J=22.4 Hz, 1H), 5.65-5.43 (m, 2H), 4.78-4.57 (m, 2H), 4.35-3.86 (m, 2H), 3.34 (dddd, J=23.9, 9.0, 5.6, 2.8 Hz, 1H), 3.15-2.86 (m, 3H), 1.38-1.16 (m, 2H), 0.95-0.78 (m, 9H), 0.62 (d, J=6.5 Hz, 11H), −0.06-−0.23 (m, 6H).

Preparation of compound 7: To a solution of 6 (3.7 g, 6.91 mmol) in DCM (70 mL). The solution was added collidine (1.7 g, 13.8 mmol), DMTrCl (4.2 g, 12.4 mmol) and AgNO₃ (1.8 g, 10.4 mmol). The mixture was stirred at 25° C. for 2 h. LCMS showed 6 was consumed completely. The solvent was filtered and washed with water twice and filtered to get crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 7 (5.3 g, 6.3 mmol, 91.6% yield) as a solid. ESI-LCMS: m/z 838.4 [M+H]⁺.

Preparation of compound 8: To a solution of 7 (5.3 g, 6.3 mmol) in THF (50 mL) and added 1M TBAF (9.4 ml, 9.5 mmol). The mixture was stirred at r.t. for 24 h. LCMS showed 7 was consumed completely. H₂O was added to the mixture. Then filtered and the solution diluted with EA. The organic layer was washed with NaHCO₃ and brine. The solvent was concentrated under reduced pressure and filtered to get a crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=9/1; Detector, UV 254 nm, filtered to get 8 (4.0 g, 5.5 mmol, 87.4% yield) as a solid. ESI-LCMS: m/z 724.3 [M+H]⁺; ¹H NMR (600 MHz, DMSO-d6) δ 12.10 (d, J=5.9 Hz, 1H), 11.75-11.51 (m, 1H), 8.07 (d, J=185.6 Hz, 1H), 7.53-7.09 (m, 8H), 6.90-6.72 (m, 4H), 5.94-5.70 (m, 1H), 5.43 (dddt, J=45.0, 16.9, 10.2, 6.5 Hz, 1H), 5.19 (dd, J=7.8, 5.5 Hz, 1H), 4.83-4.60 (m, 2H), 4.37 (dq, J=23.6, 4.1, 2.8 Hz, 2H), 3.92 (ddd, J=41.2, 6.1, 3.3 Hz, 1H), 3.80-3.62 (m, 6H), 3.53-3.21 (m, 4H), 2.87-2.67 (m, J=6.7 Hz, 1H), 1.74 (ttd, J=64.9, 14.9, 13.6, 8.0 Hz, 2H), 1.43-1.20 (m, 2H), 1.20-1.01 (m, 8H).

Preparation of compound 274: To a solution of 8 (2.8 g, 3.87 mmol) in DCM (30 mL) was added DCI (390 mg, 3.29 mmol) and CEP[N(iPr)₂]₂ (1.5 g, 5.03 mmol) under N₂ pro. The mixture was stirred at 20° C. for 1.5 h. LCMS showed 8 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 274 (3.1 g, 3.4 mmol, 86.7% yield) as a white solid. ESI-LCMS: m/z 924.2 [M+H]⁺; H NMR (400 MHz, DMSO-d6) δ 12.14 (8, 1H), 11.61 (d, J=20.9 Hz, 1H), 8.39-7.68 (m, 1H), 7.53-7.12 (m, 9H), 6.86 (dt, J=11.0, 7.1 Hz, 4H), 5.97-5.67 (m, 1H), 5.57-5.18 (m, 1H), 4.84-4.34 (m, 3H), 4.22-3.92 (m, 2H), 3.86-3.47 (m, 11H), 3.46-3.18 (m, 4H), 2.77 (qd, J=10.7, 10.2, 6.3 Hz, 2H), 2.66-2.35 (m, 2H), 1.96-1.64 (m, 1H), 1.58-0.91 (m, 20H); ³¹P NMR (162 MHz, DMSO-d6) δ 150.08, 149.81, 148.89.

### Preparation of Compound 275

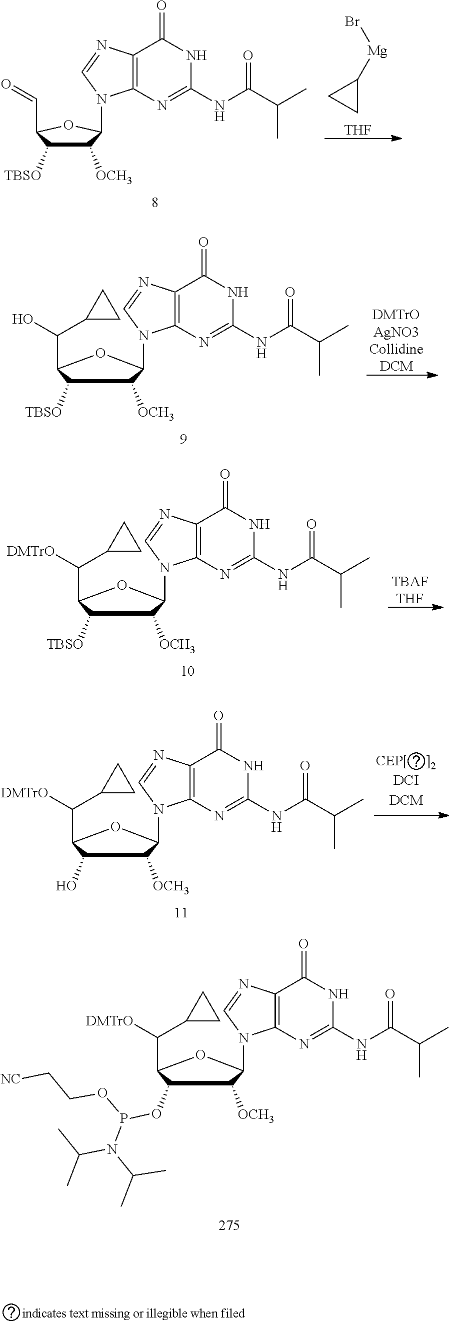

Preparation of compound 9: To a solution of bromo (cyclopropyl) magnesium (1M, 14.5 g, 100 mmol) in THF (67 mL), $ (12 g, 25 mmol) in THE (anhydrous, 70 mL) was added dropwise at 0° C. in N₂. The solution was stirred 4 hours at r.t. The reaction was worked 30% by LCMS. Added aqueous NH₄Cl (50 mL), and EA (300 mL), washed with aqueous NH₄Cl (3×500 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O) (0.5% NH₄HCO₃)=1/1 increasing to CH₃CN/H₂O (0.5% NHHCO₃)=1/0 within 25 min, the eluted product was collected at CH₃CN/H₂O (0.5% NH₄HCO₃)=2/1; Detector, UV 254 nm, filtered to get 9 (3.0 g, 5.46 mmol, 21.8% yield) as a white solid. ¹H NMR (400 MHz, DMSO-d6) δ 12.01 (d, J=2.8 Hz, 1H), 11.53 (d, J=1.6 Hz, 1H), 8.27 (d, J=31.7 Hz, 1H), 5.19 (s, 1H), 4.56-4.08 (m, 2H), 3.89-3.75 (m, 1H), 3.17 (d, J=18.8 Hz, 3H), 2.90 (ddd, J=55.4, 8.0, 3.4 Hz, 1H), 2.68 (pd, J=6.8, 2.7 Hz, 1H), 1.02 (dd, J=6.9, 1.5 Hz, 7H), 0.91-0.58 (m, 10H), 0.42-−0.08 (m, 9H). ESI-LCMS m/z 522.5 [M+H]⁺

Preparation of compound 10: To a solution of 9 (3 g, 5.8 mmol) and DMTrCl (3.5 g, 10.4 mmol) in DCM (30 mL), AgNO₃ (1.5 g, 8.6 mmol) and collidine (1.4 g, 11.5 mmol) was added at r.t in N₂. The solution was stirred 1 hour at r.t. 9 was consumed by LCMS. The reaction was diluted with DCM and filtered. The combined filtrate was washed with water (3×100 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 10 (4.4 g, 5.2 mmol, 91.0% yield) as a white solid. ESI-LCMS m/z 824.5 [M+H]⁺

Preparation of compound 11: To a solution of 10 (4.3 g, 5.2 mmol) in THF (43 mL), TBAF (10 mL, 10.0 mmol) was added at r.t in N₂. The solution was stirred 16 hours at r.t. The reaction was worked well by LCMS. The reaction was diluted with EA and washed with water (3×200 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)-1/0 within 25 min, the eluted product was collected at CH₃CN/H₂O (0.5% NH₄HCO₃)=9/1; Detector, UV 254 nm, filtered to get 11 (3.3 g, 4.6 mmol, 87.3% yield) as a white solid. ¹H NMR (600 MHz, DMSO-d₆) δ 12.10 (d, J=8.8 Hz, 1H), 11.71 (d, J=12.4 Hz, 1H), 7.46 (dd, J=8.0, 4.1 Hz, 2H), 7.38-7.16 (m, 7H), 6.92-6.78 (m, 4H), 5.76 (dd, J=31.4, 7.3 Hz, 1H), 5.18 (dd, J=41.8, 5.0 Hz, 1H), 4.68-4.50 (m, 1H), 4.23 (ddd, J=160.7, 7.5, 5.1 Hz, 1H), 3.94-3.51 (m, 7H), 3.37 (d, J=16.9 Hz, 3H), 2.87-2.59 (m, 2H), 1.99 (s, 2H), 1.13 (dt, J=6.8, 4.5 Hz, 6H), 0.64-−0.45 (m, 4H). ESI-LCMS m/z 710.1 [M+H]⁺

Preparation of compound 275: To a solution of 11 (2.7 g, 3.8 mmol) in DCM (27 mL), CEP[N(iPr)₂]₂ (1.4 g, 4.6 mmol) and DCI (359.4 mg, 3.0 mmol) was added rapidly at r.t in N₂. The solution was stirred 1 hour at r.t. 11 was consumed by LCMS. The reaction mixture was diluted with DCM (200 mL), the solution was washed with water (3×100 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 275 (3.1 g, 3.4 mmol, 88.7% yield) as a white solid. ¹H NMR (600 MHz, DMSO-d₆) δ 11.69 (d, J=265.3 Hz, 2H), 8.10 (s, OH), 7.33-6.92 (m, 9H), 6.80 (s, OH), 6.68-6.52 (m, 4H), 5.66-5.36 (m, 1H), 4.78 (dd, J=10.5, 5.0 Hz, OH), 4.30 (ddd, J=13.6, 8.4, 4.6 Hz, 1H), 4.14-3.84 (m, 1H), 3.66-3.28 (m, 10H), 3.18-3.02 (m, 3H), 2.55 (tq, J=14.0, 7.0, 6.4 Hz, 3H), 2.42-2.08 (m, 2H), 1.75 (s, 1H), 1.13-0.81 (m, 20H), 0.28 (ddt, J=19.4, 9.5, 4.8 Hz, 1H), −0.01 (dq, J=8.3, 4.5, 3.6 Hz, 1H), −0.32 (dq, J=29.9, 5.0 Hz, 1H), −0.60 (ddd, J=39.6, 9.9, 5.0 Hz, 1H). ³¹P NMR (243 MHz, DMSO-d₆) δ 150.69, 150.18, 148.9, 148.83. ESI-LCMS m/z 910.5 [M+H]⁺.

### Example 25—Preparation of Compound 277

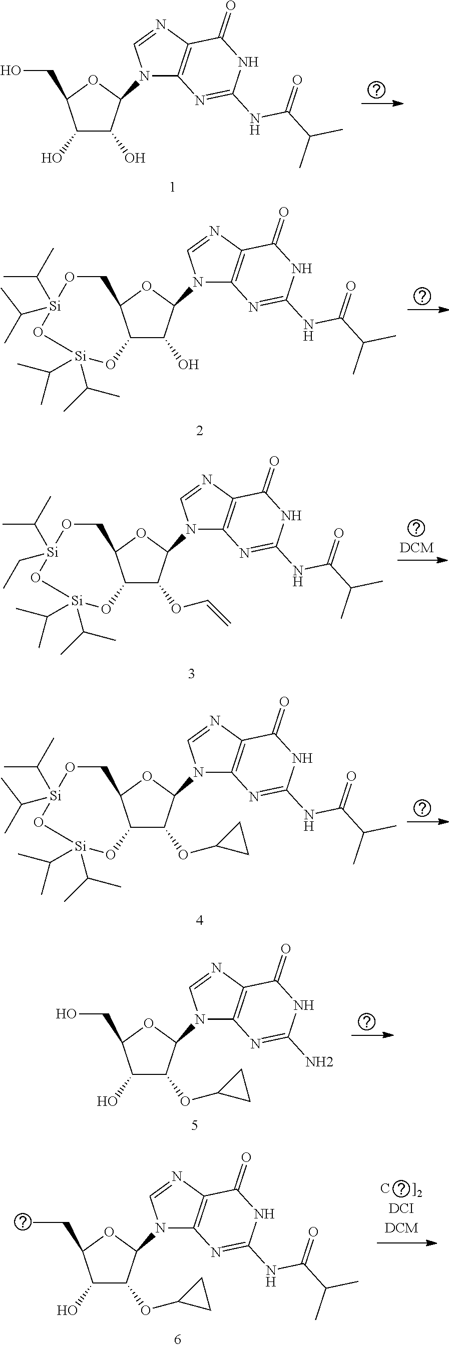

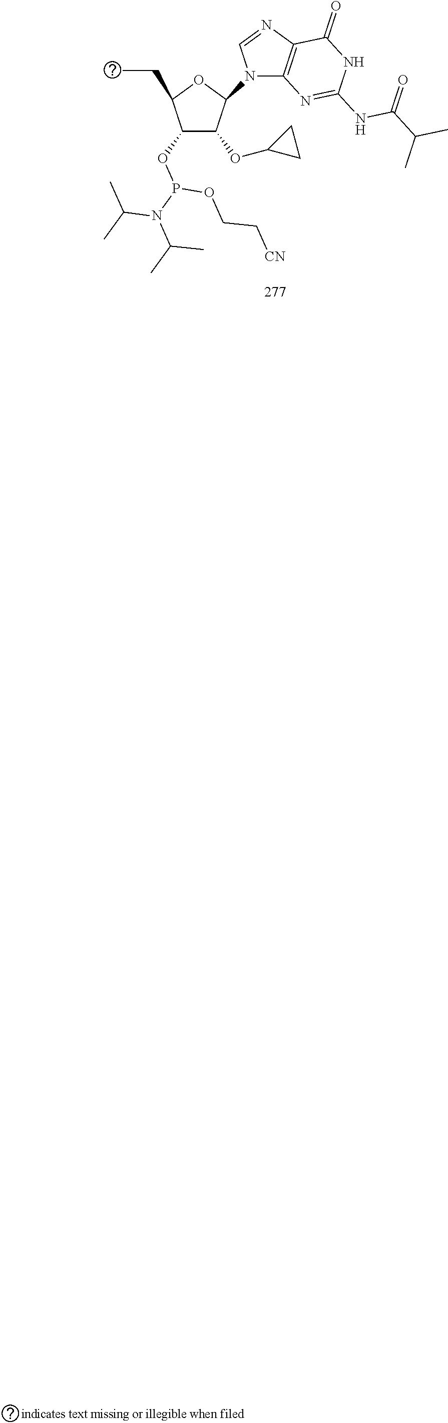 [SMILES: CC[C@H]1O[C@@H](n2cnc3c(=O)[nH]c(NC(=O)C(C)C)nc32)[C@H](OC2CC2)[C@@H]1OP(OCCC#N)N(C(C)C)C(C)C]

Preparation of compound 2: To a solution of 1 (25.0 g, 66.0 mmol) in DMF (250 mL). Then the mixture was added Imidazole (11.2 g, 164.9 mmol), TiPSCl₂ (27.0 g, 85.8 mmol) under N₂ atmosphere. The mixture was stirred at r.t. for 6 h. Then the solution was diluted with BA, washed with water twice. The solvent was concentrated under reduced pressure to give the crude. The crude was purified by silica gel; mobile phase, DCM/MeOH=100/1 increasing to DCM/MeOH=20/1, the eluted product was collected at DCM/MeOH=30/1; filtered to get 2 (28 g, 47.1 mmol, 71.5% yield) as a solid. ESI-LCMS: m/z 596.2 [M+H]⁺.

Preparation of compound 3: To a solution of 2 (28 g, 47.1 mmol) in DCM (300 mL). The solution was added PPTS (2.4 g, 9.4 mmol), EVE (44.1 g, 611 mmol). The mixture was stirred at 25° C. for 16 h. LCMS showed 2 was consumed completely. The solvent was washed with water twice and filtered to get the crude 2-1. ESI-LCMS: m/z 668 [M+H]⁺.

To a solution of 2-1 and TEA (61.8 g, 661 mmol) in DOM (300 ml) was added cooled to 0° C. The solution was added TMSOTf (104.6 g, 471 mmol) drop wise at 0° C. under argon atmosphere. The mixture was stirred at 25° C. for 16 h. LCMS showed 2-1 was consumed completely. The solvent was washed with water twice and filtered to get the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 3 (23.5 g, 33.8 mmol, 80% yield) as a solid. ESI-LCMS: m/z 662.7 [M+H]⁺.

Preparation of compound 4: To a solution of 1M Et₂Zn (96 mL, 96.4 mmol) and DIM (25.8 g, 96.40 mmol) in DCM (225 mL) was stirred at 0° C. for 10 min. The mixture was added 3 (15 g, 24.1 mmol) drop wise under argon atmosphere. The mixture was stirred at 25° C. for 6 h. NH₄Cl (aq.) was added to the mixture. Then filtered and the solution diluted with EA. The organic layer was washed with water and brine. The organic layer was concentrated under reduced pressure and filtered to get a crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 4 (3.2 g, 5.03 mmol, 20.1% yield) as a solid. ESI-LCMS: m/z 636.3 [M+H]⁺;

Preparation of compound 5: To a solution of 4 (3.2 g, 5.0 mmol) in THF (30 mL) and added 1M TBAF (9 mL, 7.5 mmol). The mixture was stirred at r.t. for 4 h. LCMS showed 4 was consumed completely. H₂O was added to the mixture. Then filtered and the solution diluted with EA. The organic layer was washed with NaHCO₃ (aq.) and brine. The solvent was concentrated under reduced pressure and filtered to get a crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=3/2; Detector, UV 254 nm, filtered to get 5 (1.8 g, 4.56 mmol, 90.8% yield) as a solid. ESI-LCMS: m/z 394 [M+H]⁺;

Preparation of compound 6: To a stirred mixture of 5 (1.8 g, 4.56 mmol) in pyridine (20 mL) was added DMTrCl (1.9 g, 5.5 mmol) at room temperature under argon atmosphere. The resulting mixture was stirred for 3 h at room temperature under argon atmosphere. The reaction was quenched by the addition of saturated NaHCO₃. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE (0.5% TEA)/EtOAc (2:3) to afford 6 (2.8 g, 4.01 mmol, 88.1%) as an off-white solid.

ESI-LCMS: m/z 696 [M+H]⁺;

Preparation of compound 277: To a solution of 6 (2.3 g, 3.29 mmol) in DCM (25 mL) was added DCI (331.2 mg, 2.8 mmol) and CEP[N(iPr)₂]₂ (1.2 g, 3.95 mmol) under N₂. The mixture was stirred at 20° C. for 2 h. LCMS showed 6 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 277 (2.5 g, 2.79 mmol, 84.7% yield) as a white solid. ESI-LCMS: m/z 897 [M+H]⁺; ¹H NMR (400 MHz, DMSO-d6) δ 12.12 (s, 1H), 11.58 (s, 1H), 8.16 (s, 1H), 7.49-7.07 (m, 9H), 6.97-6.69 (m, 4H), 5.93 (dd, J=14.3, 6.7 Hz, 1H), 4.79 (dd, J=6.8, 5.0 Hz, 1H), 4.50 (dtd, J=10.4, 5.0, 2.9 Hz, 1H), 4.23 (dt, J=5.7, 3.3 Hz, 1H), 3.91-3.12 (m, 13H), 2.87-2.69 (m, 2H), 2.62 (t, J=5.9 Hz, 1H), 1.28-0.87 (m, 19H), 0.70-0.21 (m, 4H). ³¹P NMR (162 MHz, DMSO-d6) δ 149.69, 149.36.

### Example 26—Preparation of 2′O-CH₂Cyp-G & 3′O-CH₂Cyp-G Precursor

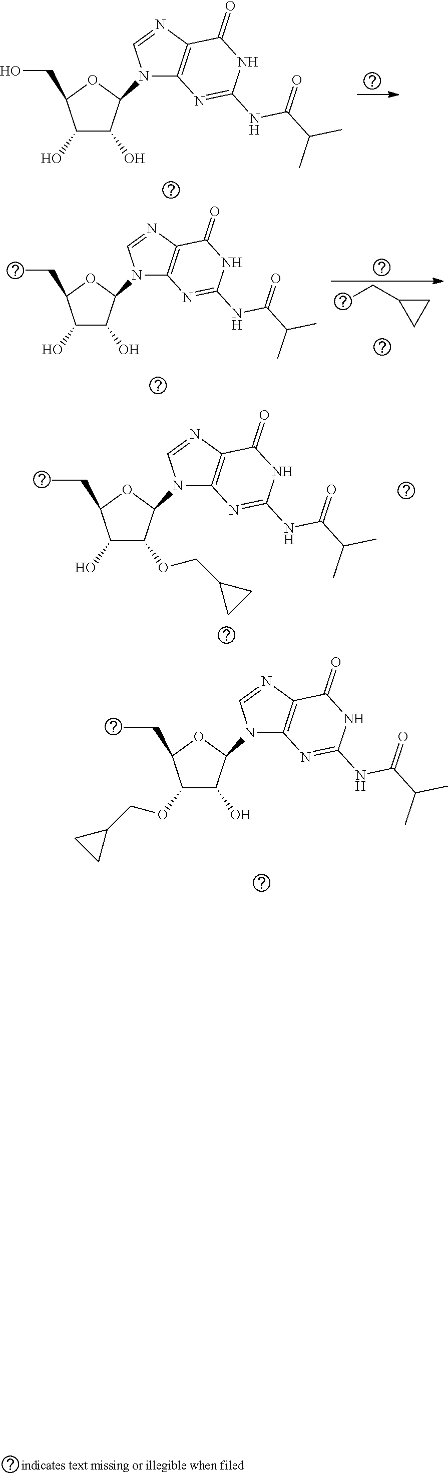

Preparation of compound 2: To a solution of 1 (100.0 g, 283.0 mmol) in pyridine (1000 mL) added DMTrCl (114.8 g, 339.6 mmol). Then the solution was stirred at r.t for 2 h. LC-MS showed 1 was consumed completely. Then the solution was quench with NaHCO₃ (aq.) and diluted with EA, washed with water twice and brine. The organic phase dried over anhydrous Na₂SO₄, filtered and concentrated under reduced pressure. This resulted was purified by silica gel column chromatograph (eluent, DCM/MeOH=100:1˜20:1). This resulted in 2 (140 g, 213.5 mmol, 60.6% yield, 98% purity) as a white solid. ESI-LCMS: m/z 656.3 [M+H]⁺.

Preparation of compound 3a and 3b: To a solution of 2a (82.3 g, 610.0 mmol) in ACN/toluene=1:1 (1000 mL) added Dibutyltin oxide (56.9 g, 228.7 mmol), TBAI (56.3 g, 152.5 mmol), 4A molecular sieve (10.0 g) at r.t. Then added 2 (100.0 g, 152.5 mmol) at r.t under N₂. Then the solution was stirred at 80° C. for 16 h. LC-MS discovered 3a and 3b. Then the solution was diluted with EA, washed with water twice and brine. The organic phase dried over anhydrous Na₂SO₄, filtered and concentrated under reduced pressure. This resulted was purified by silica gel column chromatograph (eluent, DCM/EA=10:1˜1:9). This resulted in 3a (10 g, 90% purity) as a white solid and 3b (4 g, 90% purity) as a white solid. Then the product was purified by prep-SFC separation to give 3a (8.0 g, 11.2 mmol, 7.4% yield, 99% purity) and 3b (1.2 g, 1.6 mmol, 1.0% yield, 99% purity) as a white solid.

3a ESI-LCMS: m/z 710.2 [M+H]⁺. ¹H NMR (400 MHz, DMSO-d₆) δ 11.95 (s, 1H), 11.48 (s, 1H), 8.01 (s, 1H), 7.28-6.98 (m, 9H), 6.82-6.56 (m, 4H), 5.82 (d, J=5.4 Hz, 1H), 5.02 (d, J=5.8 Hz, 1H), 4.31 (t, J=5.3 Hz, 1H), 4.24-4.02 (m, 1H), 4.00-3.84 (m, 1H), 3.57 (d, J=0.9 Hz, 6H), 3.28 (dd, J=10.4, 7.0 Hz, 1H), 3.25-3.17 (m, 1H), 3.13 (dd, J=10.4, 6.0 Hz, 1H), 3.03 (dd, J=10.4, 3.4 Hz, 1H), 2.62 (hept, J=6.8 Hz, 1H), 0.98 (dd, J=6.9, 2.1 Hz, 6H), 0.84 (s, 1H), 0.34-0.18 (m, 2H).

3b ESI-LCMS: m/z 710.2 [M+H]⁺, ¹H NMR (400 MHz, DMSO-dc) δ 11.96 (s, 1H), 11.56 (s, 1H), 8.02 (s, 1H), 7.30-6.93 (m, 9H), 6.80-6.53 (m, 4H), 5.72 (d, J=4.8 Hz, 1H), 5.39 (d, J=5.8 Hz, 1H), 3.93 (q, J=1.9 Hz, 2H), 3.59 (s, 6H), 3.32 (dd, J=10.3, 6.7 Hz, 1H), 3.12 (ddd, J=15.4, 11.3, 7.2 Hz, 3H), 2.63 (h, J=6.8 Hz, 1H), 0.99 (d, J=6.8 Hz, 6H), 0.90-0.76 (m, 1H), 0.28 (ddd, J=7.9, 4.6, 3.2 Hz, 2H).

### Example 27—Preparation of Compound 278

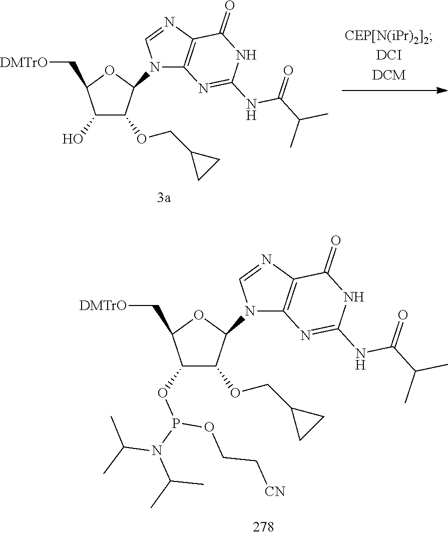

Preparation of compound 278: To a solution of 3a (5.2 g, 7.3 mmol) in DCM (52 mL) was added DCI (734 mg, 6.2 mmol) and CEP[N(iPr)₂]₂ (3.08 g, 10.2 mmol) under N₂. The mixture was stirred at r.t for 1 h. LCMS showed 3a was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NHAHCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 278 (5.3 g, 5.8 mmol, 79.4% yield, 99.0% purity) as a white solid. ESI-LCMS: m/z 910.2 [M+H]⁺. H NMR (400 MHz, DMSO-d₆) δ 11.75 (d, J=19.6, 8 Hz, 2H), 8.05 (s, 1H), 7.35-7.02 (m, 9H), 6.72 (dt, J=8.8, 4.4 Hz, 4H), 5.81 (dd, J=15.6, 6.4 Hz, (H), 4.54 (dd, J=6.5, 5.0 Hz, 1H), 4.31 (ddd, J=10.1, 5.2, 3.2 Hz, 1H), 4.12 (dd, J=5.3, 3.2 Hz, 1H), 3.77-3.36 (m, 10H), 3.21 (dddt, J=30.5, 20.1, 14.8, 5.6 Hz, 4H), 2.76-2.43 (m, 3H), 1.12-0.87 (m, 18H), 0.82 (ddt, J=13.0, 8.0, 4.2 Hz, 1H), 0.35-0.19 (m, 2H). ³¹P NMR (162 MHz, DMSO-d₆) δ 149.47, 149.26.

### Example 28—Preparation of Compound 327

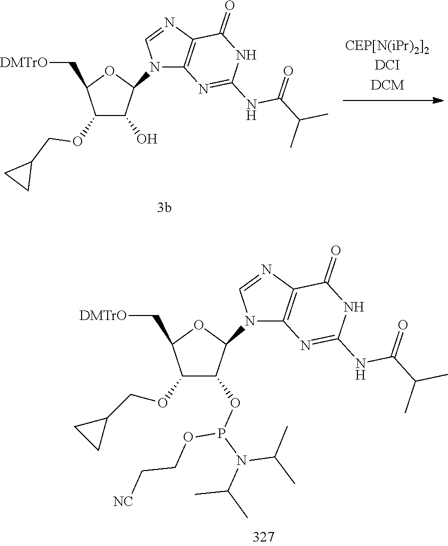

Preparation of compound 327: To a solution of 3b (1.0 g, 1.4 mmol) in DCM (10 mL) was added DCI (141 mg, 1.2 mmol) and CEP[N(iPr)₂]₂ (593 mg, 1.9 mmol) under N₂. The mixture was stirred at rt for 1 h. LCMS showed 3b was consumed completely. The product was extracted with DOM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O) (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 327 (1.0 g, 1.1 mmol, 78.5% yield, 99% purity) as a white solid. ESI-LCMS: m/z 910.2 [M+H]⁺. ¹H NMR (400 MHz, DMSO-d₆) δ 11.93 (s, 1H), 11.51 (s, 1H), 8.02 (d, J=3.6 Hz, 1H), 7.26-6.96 (m, 9H), 6.70 (dt, J=9.1, 2.7 Hz, 4H), 5.85 (dd, J=21.4, 5.7 Hz, 1H), 4.81 (ddt, J=10.0, 7.5, 4.8 Hz, 1H), 4.11-3.85 (m, 2H), 3.63 (dddd, J=11.5, 10.0, 5.2, 2.5 Hz, 7H), 3.34 (tddd, J=19.1, 12.3, 10.3, 6.7 Hz, 4H), 3.24-3.07 (m, 3H), 2.69-2.51 (m, 2H), 2.47-2.37 (m, 1H), 1.05-0.64 (m, 19H), 0.34-0.21 (m, 2H). ³¹P NMR (162 MHz, DMSO-d₆) δ 150.28, 150.25.

### Example 29—Preparation of compound 2′O-CH₂Cyp-A & 3′O-CH₂Cyp-A precursor

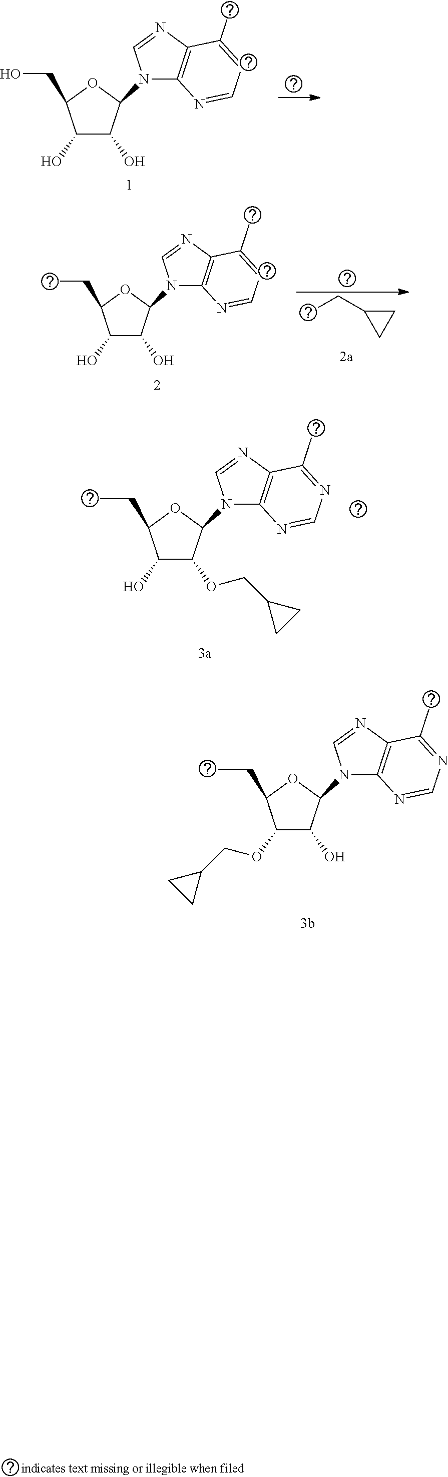

Preparation of compound 2: To a solution of 1 (100.0 g, 269.2 mmol) in pyridine (1000 mL) added DMTrCl (90.9 g, 323.0 mmol). Then the solution was stirred at r.t for 2 h. LC-MS showed 1 was consumed completely. Then the solution was quench with NaHCO₃ (aq.) and diluted with EA, washed with water twice and brine. The organic phase dried over anhydrous Na₂SO₄, filtered and concentrated under reduced pressure. This resulted was purified by silica gel column chromatograph (eluent, DCM/MeOH=100:1˜20:1). This resulted in 2 (143 g, 212.1 mmol, 78.8% yield, 96.0% purity) as a white solid. ESI-LCMS: m/z 674.3 [M+H]⁺.

Preparation of compound 3a and 3b: To a solution of 2a (100.0 g, 593.5 mmol) in ACN/toluene=1:1 (1000 mL) added Dibutyltin oxide (90.9 g, 222.5 mmol), TBAI (90.9 g, 148.4 mmol), 4A molecular sieve (10.0 g) at r.t. Then added 2 (100 g, 148.4 mmol) at r.t at N₂. Then the solution was stirred at 80° C. for 16 h. LC-MS discovered 3a and 3b. Then the solution was diluted with EA, washed with water twice and brine. The organic phase dried over anhydrous Na₂SO₄, filtered and concentrated under reduced pressure. This resulted was purified by silica gel column chromatograph (eluent, PE/EA=10:1˜2:1). This resulted in 3a (13.0 g, 92.0% purity) as a white solid and 3b (11.0 g, 80% purity) as a white solid. Then the product was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=1/0 within 40 min, the eluted product was collected at CH₃CN/H₂O (0.05% NH₄HCO₃)=3/2; Detector, UV 254 nm. This resulted in 3a (8.2 g, 11.3 mmol, 7.6% yield, 99.0% purity) and 3b (6.0 g, 8.2 mmol, 5.5% yield, 99.0% purity) as a white solid.

3a ESI-LCMS: m/z 728.3 [M+H]⁺. ¹H NMR (600 MHz, DMSO-d₆) δ 11.24 (s, 1H), 8.67 (d, J=35.6 Hz, 2H), 8.06 (d, J=7.7 Hz, 2H), 7.64 (t, J=7.4 Hz, 1H), 7.55 (t, J=7.6 Hz, 2H), 7.38 (d, J=7.8 Hz, 2H), 7.32-7.16 (m, 7H), 6.95-6.76 (m, 4H), 6.20 (d, J=5.0 Hz, 1H), 5.75 (s, 1H), 5.28 (d, J=6.2 Hz, 1H), 4.74 (t, J=5.1 Hz, 1H), 4.46 (q, J=5.3 Hz, 1H), 4.16 (q, J=4.7 Hz, 1H), 3.72 (8, 6H), 3.55-3.16 (m, 5H), 0.99 (pt, J=7.6, 5.3, 4.0 Hz, 1H), 0.46-0.30 (m, 2H), 0.14 (td, J=7.5, 6.2, 3.3 Hz, 2H).

3b ESI-LCMS: m/z 728.3 [M+H]⁺. H NMR (400 MHz, DMSO-d₆) δ 11.05 (3, 1H), 8.49 (d, J=31.4 Hz, 2H), 8.06-7.71 (m, 2H), 7.41 (dt, J=37.2, 7.5 Hz, 3H), 7.27-6.94 (m, 9H), 6.67 (dd, J=8.9, 2.8 Hz, 4H), 5.90 (d, J=4.6 Hz, 1H), 5.42 (d, J=5.9 Hz, 1H), 4.79 (q, J=5.1 Hz, 1H), 4.06 (dt, J=40.3, 4.8 Hz, 2H), 3.33 (dd, J=10.4, 6.7 Hz, 1H), 3.12 (ddd, J=25.1, 11.9, 6.0 Hz, 3H), 0.85 (dq, J=12.6, 5.7 Hz, 1H), 0.26 (qd, J=7.8, 4.8 Hz, 2H).

### Example 30—Preparation of Compound 279

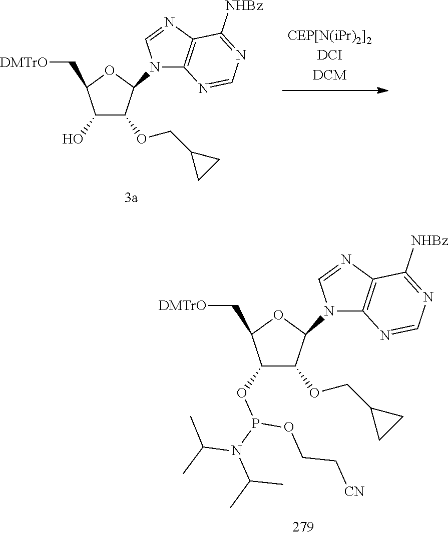

Preparation of compound 279: To a solution of 3a (5.2 g, 7.1 mmol) in DCM (52 mL) was added DCI (716 mg, 6.1 mmol) and CEP[N(iPr)₂]₂ (2.58 g, 8.6 mmol) under N₂. The mixture was stirred at r.t for 1 h. LCMS showed 3a was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₂HCO₃)=1/0 within 25 min, the eluted product was collected at CH₃CN/H₂O (0.05% NH₄HCO₃)=1/0; Detector, UV 254 nm. This resulted in 279 (5.1 g, 5.5 mmol, 77.5% yield, 99.0% purity) as a white solid. ESI-LCMS: m/z 928.2 [M+H]⁺. ¹H NMR (400 MHz, DMSO-d₆) δ 11.23 (s, 1H), 8.73-8.54 (m, 2H), 8.17-7.95 (m, 2H), 7.72-7.48 (m, 3H), 7.37 (ddd, J=8.2, 5.5, 1.4 Hz, 2H), 7.31-7.15 (m, 7H), 6.90-6.76 (m, 4H), 6.19 (dd, J=10.3, 5.2 Hz, 1H), 4.95 (t, J=5.1 Hz, 1H), 4.80-4.61 (m, 1H), 4.27 (dq, J=24.8, 4.6 Hz, 1H), 3.99-3.78 (m, 1H), 3.75-3.55 (m, 9H), 3.46-3.35 (m, 3H), 3.27 (ddd, J=10.4, 7.8, 5.5 Hz, 1H), 2.80 (td, J=5.7, 1.1 Hz, 1H), 2.62 (L, J=5.9 Hz, 1H), 1.15 (t, J=6.5 Hz, 9H), 1.05 (d, J=6.7 Hz, 3H), 0.38 (dtt, J=6.6, 2.5, 1.4 Hz, 2H), 0.12 (dtt, J=6.3, 3.5, 1.8 Hz, 2H), 3P NMR (162 MHz, DMSO-d₆) δ 149.24, 248.87.

### Example 31—Preparation of Compound 325

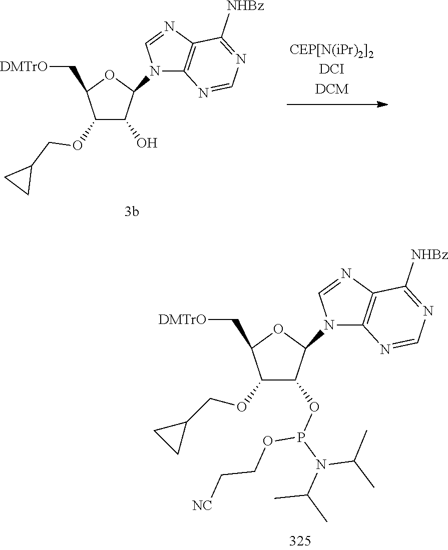

Preparation of compound 325: To a solution of 3b (5.2 g, 7.1 mmol) in DCM (52 mL) was added DCI (716 mg, 6.1 mmol) and CEP[N(Pt)₂]₂ (2.58 g, 8.6 mmol) under N₂. The mixture was stirred at r.t for 1 h. LCMS showed 3b was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 am. This resulted in 325 (5.1 g, 5.5 mmol, 77.5% yield, 99.0% purity) as a white solid. ESI-LCMS: m/z 928.2 [M+H]⁺. ¹H NMR (400 MHz, DMSO-d₆) δ 11.04 (s, 1H), 8.66-8.27 (m, 2H), 7.97-7.75 (m, 2H), 7.37 (td, J=7.4, 13 Hz, 3H), 7.25-6.92 (m, 9H), 6.68 (dt, J=9.0, 1.9 Hz, 4H), 6.04 (dd, J=23.3, 4.6 Hz, 1H), 5.19-4.91 (m, 1H), 4.23 (dt, J=9.8, 5.0 Hz, 1H), 4.03 (dq, J=15.2, 4.5 Hz, 1H), 3.54 (S, 7H), 3.46-3.25 (m, 4H), 3.26-3.16 (m, 2H), 3.09 (ddd, J=10.5, 4.5, 2.9 Hz, 1H), 2.60 (dd, J=6.6, 5.3 Hz, 1H), 2.41 (t, J=6.0 Hz, 1H), 0.91 (dd, J=12.1, 6.7 Hz, 10H), 0.64 (d, J=6.7 Hz, 3H), 0.35-0.16 (m, 2H), 3P NMR (162 MHz, DMSO-d₆) § 150.12, 149.78.

### Example 32—Preparation of Compound 5′Cyp & 5′CH₂-Cyp Modified Monomers

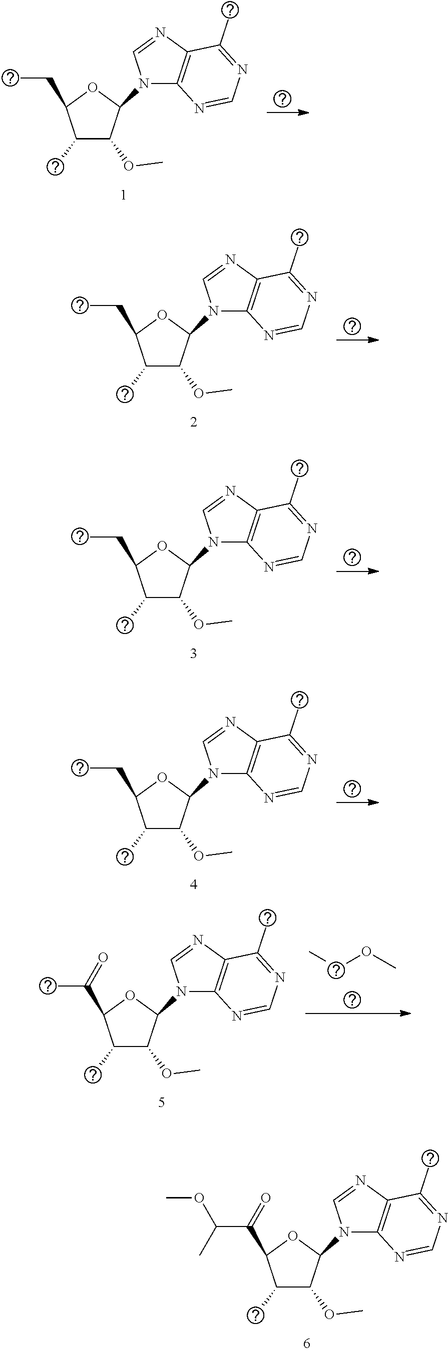

### Preparation of Intermediate 6

Preparation of compound 2: To a solution of 1 (200 g, 711.1 mmol) in DMF (1600 mL), imidazole (169.4 g, 2.5 mol) and TBSCl (146.4 g, 1.8 mol) was added at r.t. The solution was stirred 16 hours at r.t. LCMS showed 1 was consumed. The solution was added EA (3 L), washed with water (2×8 L), and aqueous NaCl (2 L). The organic phase dried over anhydrous Na₂SO₄, concentrated by rotary evaporator to give crude. The organic phase dried over anhydrous Na₂SO₄, concentrated by rotary evaporator to give crude 2 (550 g, 679.7 mmol, 95.6% yield) as a white solid. ESI-LCMS m/z 510.2 [M+H]⁺

Preparation of compound 3: To a solution of 2 (550 g, 679.7 mmol) in pyridine (2000 mL), BzCl (191.0 g, 1.4 mol) was added at 0° C. The solution was stirred 1 h at r.t. LCMS showed 1 was consumed. The solution was diluted with EA (3 L), washed with water (2×10 L), and aqueous NaCl (2 L). The organic phase dried over anhydrous Na₂SO₄, concentrated by rotary evaporator to give crude 3 (600 g, 654.84 mmol, 96.34% yield) as a white solid. The crude was used to next step directly. ESI-LCMS m/z 614.5 [M+H]⁺

Preparation of compound 4: To a solution of 3 (600 g, 654.8 mmol) in THF (2 L), TFA: H₂O=1:1 (860 mL) was added at 0° C. The solution was stirred 2 hours at 0° C. LCMS showed 3 was consumed. Con·NH₄OH was at 0° C. to adjust pH=8. The solution was added EA (3 L), washed with water (2×10 L), and aqueous NaCl (2 L). The organic phase dried over anhydrous Na₂SO₄, concentrated by rotary evaporator to give crude. The crude was purified by silica gel; mobile phase, DCM/MeOH=200/1 increasing to DCM/MeOH=20/1, the eluted product was collected at DCM/MeOH=50/1 to give 4 (248 g, 466.58 mmol, 71.25% yield) as a white solid. ¹H NMR (400 MHz, DMSO-d₆) δ 11.10 (s, 1H), 8.64 (d, J=2.8 Hz, 2H), 8.01-7.84 (m, 2H), 7.55-7.32 (m, 3H), 6.03 (d, J=5.9 Hz, 1H), 4.63-4.27 (m, 2H), 3.87 (q, J=3.9 Hz, 1H), 3.67-3.35 (m, 2H), 0.79 (s, 9H), 0.00 (d, J=1.8 Hz, 6H). ESI-LCMS m/z 500.3 [M+H]⁺

Preparation of compound 5: To a solution of 4 (80 g, 160.12 mmol) in DCM (480 mL), NaHCO₃ (18.8 g, 224.2 mmol) and water (400 mL) was added at r.t. The mixture was added TEMPO (5.0 g, 32.0 mmol) at 5° C. Then the NaClO (391.7 g, 384.3 mmol) was added dropwise at 5° C. The mixture was stirred 1 hour at 15° C. LCMS showed 4 was consumed. The mixture was cooling to 5° C., the solid was collected by filtration. The filter cake was rinsed with water (50 ml) and DCM (2×500 mL), drying by rotary evaporator to give crude 5 (75 g, 138.7 mmol, 86.6% yield) as a yellow solid. The crude was used to next step directly. ESI-LCMS m/z . 514.4 [M+H]⁺

Preparation of compound 6: To a solution of 5 (73 g, 142.1 mmol) and N,O-dimethylhydroxylamine hydrochloride (15.9 g, 163.4 mmol) in pyridine (75 mL) and DMF (240 mL). Then T₃P (135.59 g, 213.19 mmol, 50% purity) was slowly added over the course of 30 min in N₂ at 0° C. The solution was stirred 2 hours at 0° C. LCMS showed 5 was consumed. The solution was added EA (1000 ml), washed with water (2×3 L), and aqueous NaCl (1 L). The organic phase dried over anhydrous Na₂SO₄, concentrated by rotary evaporator to give crude. The crude was purified by silica gel; mobile phase, DCM/MeOH=100/1 increasing to DCM/MeOH=30/1, the eluted product was collected at DCM/MeOH=50/1 to give 6 (68 g, 118.49 mmol, 83.37% yield) as a white solid. ¹H NMR (400 MHz, DMSO-d₆) δ 11.09 (s, 1H), 8.90 (s, 1H), 8.63 (s, 1H), 7.95-7.87 (m, 2H), 7.54-7.44 (m, 1H), 7.40 (dd, J=8.3, 6.8 Hz, 2H), 6.16 (d, J=7.1 Hz, 1H), 4.82-4.68 (m, 1H), 4.59-4.53 (m, 1H), 4.27 (dd, J=7.2, 3.8 Hz, 1H), 3.57 (s, 3H), 3.12 (s, 3H), 3.05 (s, 3H), 0.79 (s, 9H), 0.00 (s, 6H). ESI-LCMS m/z 557.6 [M+H]⁺

### Preparation of Intermediate 8a and 8b

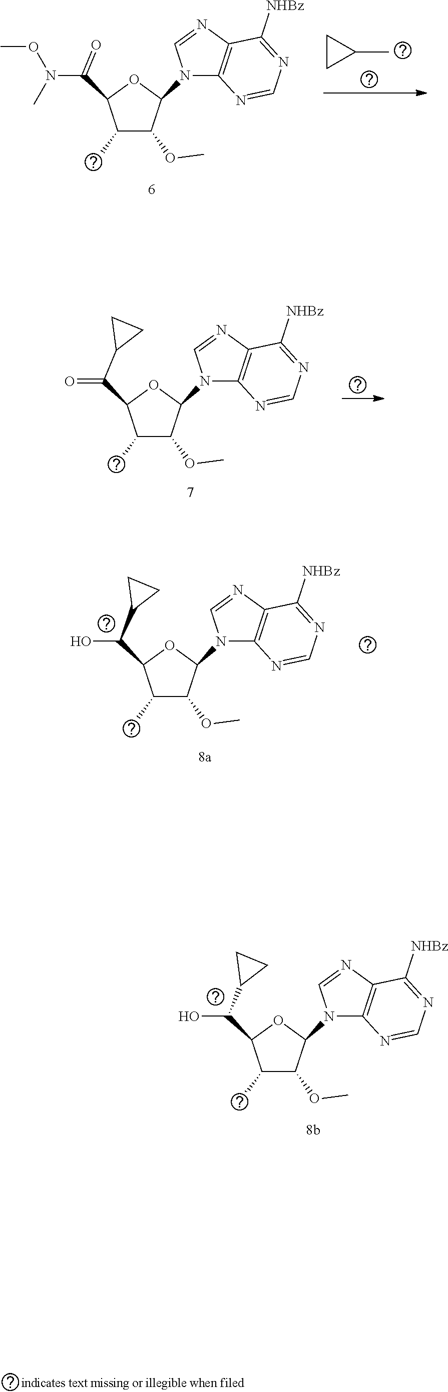

Preparation of compound 7: To a solution of 6 (14 g, 25.2 mmol) in THF (170 mL), bromo (cyclopropyl) magnesium (30 mL, 30.18 mmol) was slowly added at −20° C. in N₂. The solution was stirred 20 min at −20° C. T. The solution was stirred 1 hour at −20° C. LCMS showed 6 was consumed. The solution was quenched by addition of aqueous NH₄Cl (100 ml). The solution was added EA (500 ml), washed with water (2×1.5 L), and aqueous NaCl (500 mL). The organic phase dried over anhydrous Na₂SO₄, concentrated by rotary evaporator to give crude. The crude was purified by silica gel; mobile phase, DCM/MeOH=200/1 increasing to DCM/MeOH=100/1, the eluted product was collected at DCM/MeOH=150/1 to give 7 (12 g, 21.43 mmol, 85.2% yield) as a white solid. ¹H NMR (400 MHz, DMSO-d₆) δ 11.08 (s, 1H), 8.64 (s, 1H), 8.59 (s, 1H), 7.91-7.83 (m, 2H), 7.52-7.42 (m, 1H), 7.42-7.33 (m, 2H), 6.10 (d, J=6.0 Hz, 1H), 4.73 (dd, J=4.5, 3.3 Hz, 1H), 4.37 (dd, J=6.1, 4.5 Hz, 1H), 3.15 (s, 5H), 2.11-2.00 (m, 1H), 0.76 (8, 9H), −0.00 (d, J=3.9 Hz, 6H). ESI-LCMS m/z 538.2 [M+H]⁺

Preparation of compound 8: To a solution of 7 (12 g, 22.32 mmol) in water: DMF:DCM=4:1:1 (300 mL), HCOONa (78.14 g, 1.12 mol) was added at r.t in N₂. Then (s, s) Ru-TsDPEN (283.89 mg, 446.36 μmol) was added at r.t. The solution was stirred 16 hours at r.t. LCMS showed 7 was consumed. The solution was added EA. (200 mL), washed with water (2×500 mL), and aqueous NaCl (200 mL). The organic phase dried over anhydrous Na₂SO₄, concentrated by rotary evaporator to give crude. The crude was purified by silica gel; mobile phase, DCM/MeOH=200/1 increasing to DCM/MeOH=5 0/1, the eluted product was collected at DCM/MeOH=100/1 to mixture. The mixture was SFC column chromatographed to give 5a (5.5 g, 9.89 mmol, 44.29% yield, 97% purity) ¹H NMR (600 MHz, DMSO-d₆) δ 11.11 (s, 1H), 8.72 (s, 1H), 8.65 (s, 1H), 7.94 (d, J=7.7 Hz, 2H), 7.52 (t, J=7.3 Hz, 1H), 7.43 (t, J=7.9 Hz, 2H), 6.08 (d, J=5.8 Hz, 1H), 5.23 (d, J=5.5 Hz, 1H), 4.43 (t, J=3.6 Hz, 1H), 4.32 (t, J=5.2 Hz, 1H), 3.89 (d, J=2.9 Hz, 1H), 3.21 (d, J=1.6 Hz, 4H), 2.89 (ddd, J=8.3, 5.7, 2.6 Hz, 1H), 0.95-0.87 (m, 1H), 0.79 (d, J=1.6 Hz, 9H), 0.40-0.27 (m, 2H), 0.23 (dq, J=8.9, 4.6 Hz, 1H), 0.12-−0.09 (m, 7H). ESI-LCMS m/z 540.5 [M+H] and 8b (5.5 g, 9.89 mmol, 44.29% yield, 97% purity) ¹H NMR (600 MHz, DMSO-d₆) δ 11.25 (s, 1H), 8.78 (d, J=7.1 Hz, 2H), 8.12-7.96 (m, 2H), 7.73-7.44 (m, 3H), 6.15 (d, J=7.6 Hz, 1H), 5.53 (d, J=4.4 Hz, 1H), 4.80-4.47 (m, 2H), 4.03 (d, J=3.9 Hz, 1H), 3.32-3.09 (m, 4H), 0.95 (s, 10H), 0.49-0.06 (m, 9H). ESI-LCMS m/z 540.5 [M+H]⁺ as white solid.

### Preparation of Compound 306

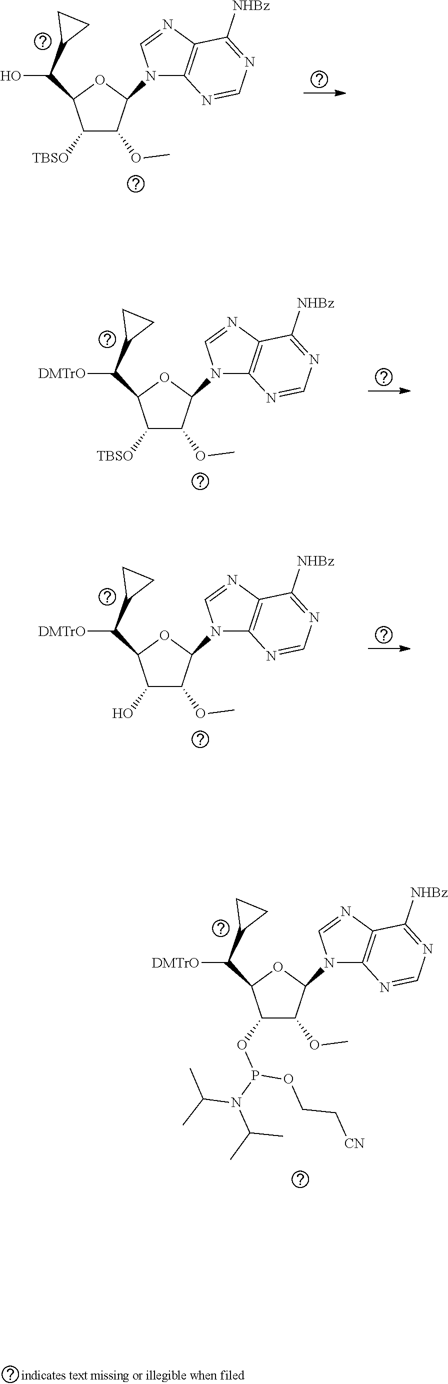

Preparation of compound 9a: To a solution of 8a (4.6 g, 8.5 mmol) in DCM (50 mL), collidine (2.8 g, 23.2 mmol) was added at r.t. under N₂. Then DMTrCl (5.6 g, 16.6 mmol) and AgNO₃ (2.4 g, 13.9 mmol) was added at r.t. The solution was stirred 4 hours at r.t. LCMS showed 8a was consumed. The solution was quenched by addition of water (20 mL) and diluted with DCM (200 mL), then filter to diatomite. The solution was washed with water (2×500 mL), and aqueous NaCl (200 mL). The organic phase dried over anhydrous Na₂SO₄, concentrated by rotary evaporator to give crude. The crude was purified by silica gel; mobile phase, PE/EA=10/1 increasing to PE/EA=1/1, the eluted product was collected at PE/EA=2/1 to give 9a (5.7 g, 6.7 mmol, 78.8% yield) as white solid. ¹H NMR (400 MHz, DMSO-d₆) § 11.21 (s, 1H), 8.69 (d, J=9.6 Hz, 2H), 8.01 (d, J=7.6 Hz, 2H), 7.61-7.40 (m, 5H), 7.39-7.04 (m, 7H), 6.83-6.74 (m, 4H), 5.94 (d, J=7.4 Hz, 1H), 4.74 (dd, J=7.6, 4.6 Hz, 1H), 4.33 (dd, J=4.5, 1.9 Hz, 1H), 3.65 (s, 7H), 3.17 (s, 3H), 2.72 (dd, J=9.0, 3.1 Hz, 1H), 0.79 (s, 9H), 0.36 (tt, J=9.0, 4.6 Hz, 1H), −0.00 (s, 5H), −0.12 (S, 3H), −0.20 (dt, J=10.0, 5.2 Hz, 1H), −0.41 (dq, J=10.1, 5.1 Hz, 1H). ESI-LCMS m/z 842.1 [M+H]⁺

Preparation of compound 10a: To a solution of 9a (5.5 g, 6.5 mmol) in THE (55 mL), TBAF (10.0 mmol, 10 mL) was added at r.t in N₂. The solution was stirred 16 hours at r.t. The reaction was worked well by LCMS. The reaction was diluted with EA (200 mL), washed with water (3×400 mL) and aqueous NaCl (200 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm, filtered to get 10a (3.7 g, 5.1 mmol, 78.4% yield) as a white solid. ¹H NMR (400 MHz, DMSO-d₆): δ −0.29-−0.26 (m, 1H), −0.12-−0.09 (m, 1H), 0.20-0.24 (m, 1H), 0.42-0.45 (m, 1H), 1.14-1.19 (m, 1H), 2.76-2.79 (m, 1H), 3.37 (s, 1H), 3.73-3.80 (m, 7H), 4.53-4.65 (m, 2H), 5.13 (d, J=6.14 Hz, 1H), 6.00 (d, J=6.73 Hz, 1H), 6.82-6.85 (m, 4H), 7.12-7.33 (m, 12H), 8.05 (d, J=7.77 Hz, 2H), 8.72 (d, J=21.23 Hz, 2H), 11.24 (s, 1H). ESI-LCMS m/z 728.1 [M+H]+

Preparation of compound 306: To a solution of 10a (3.5 g, 4.8 mmol) in DCM (35 mL), DCI (483.8 mg, 4.1 mmol) and CEP[N(iPr)₂]₂ (1.9 g, 6.2 mmol) was added rapidly at r.t in N₂. The solution was stirred 1 hour at r.t. LCMS showed 10a was consumed. The reaction was diluted with DCM and the solution was washed with water (3×100 ml). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O) (0.5% NH₄HCO₃)=1/0 within 25 min, the eluted product was collected at CH₃CN/H₂O (0.5% NH₄HCO₃)=4/1; Detector, UV 254 nm, filtered to get 306 (3.4 g, 3.7 mmol, 77.1% yield) as a white solid. ¹H NMR (400 MHz, DMSO-d₆) δ 11.29 (s, 1H), 8.82-8.25 (m, 2H), 8.15-7.99 (m, 2H), 7.70-7.11 (m, 12H), 6.94-6.76 (m, 4H), 6.09-5.94 (m, 1H), 5.75 (s, 1H), 5.01-4.83 (m, 1H), 4.74-4.41 (m, 1H), 4.09-3.49 (m, 11H), 3.28 (s, 1H), 2.93-2.78 (m, 2H), 2.69-2.50 (m, 1H), 1.29-1.03 (m, 13H), 0.50-0.33 (m, 1H), 0.21-0.07 (m, 1H), −0.02˜ ˜ 0.22 (m, 1H), −0.27-−0.42 (m, 1H). ¹H NMR (600 MHz, DMSO-d₆) δ 150.34, 149.21. ESI-LCMS m/z 928.4 [M+H]

### Preparation of Compound 305

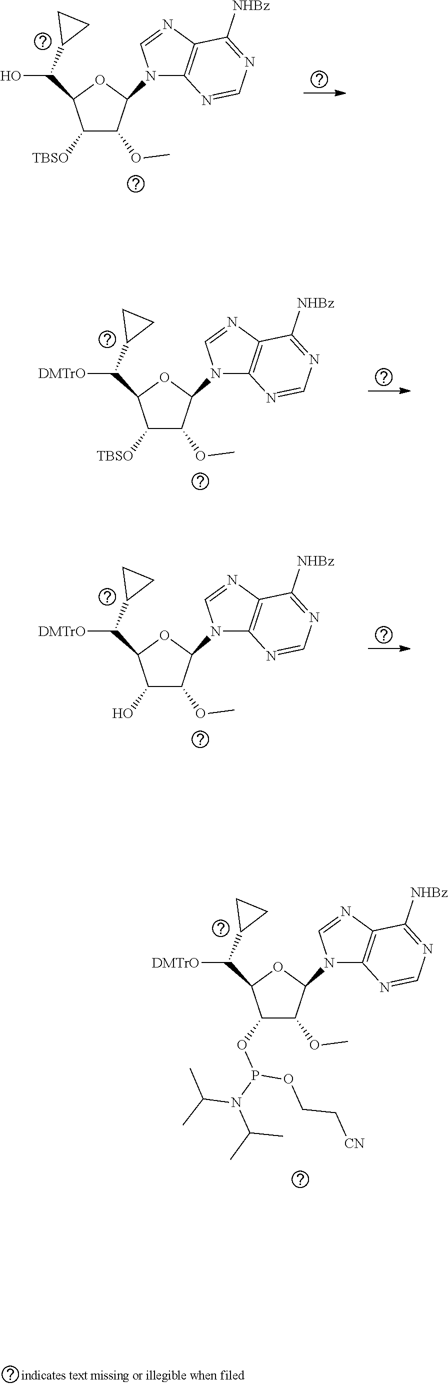

Preparation of compound 9b: To a solution of 8b (4.1 g, 7.6 mmol) in DCM (40 mL), collidine (2.8 g, 23.2 mmol) was added at r.t under N₂. Then DMTrCl (5.1 g, 15.2 mmol) and AgNO₃ (2.4 g, 13.9 mmol) was added at r.t. The solution was stirred 4 hours at r.t. LCMS showed 8b was consumed. The solution was quenched by addition of water (20 mL) and diluted with DCM (200 mL), then filter to diatomite. The solution was washed with water (2×500 mL), and aqueous NaCl (200 mL). The organic phase dried over anhydrous Na₂SO₄, concentrated by rotary evaporator to give crude. The crude was purified by silica gel; mobile phase, PE/EA=10/1 increasing to PE/EA=1/1, the eluted product was collected at PE/EA=2/1 to give 9b (5.1 g, 6.1 mmol, 80.3% yield) as white solid. ¹H NMR (400 MHz, DMSO-d₆) δ 11.13 (s, 1H), 8.61 (s, 1H), 8.21 (s, 1H), 7.99-7.92 (m, 2H), 7.61-7.51 (m, 1H), 7.51-7.36 (m, 4H), 7.30-7.1S (m, 6H), 7.15-7.06 (m, 1H), 6.82-6.73 (m, 4H), 5.90 (d, J=6.2 Hz, 1H), 4.62 (dd, J=4.6, 3.3 Hz, 1H), 4.27 (dd, J=6.3, 4.6 Hz, 1H), 3.99-3.87 (m, 1H), 3.62 (s, 6H), 3.10 (s, 3H), 2.82 (dd, J=9.1, 4.9 Hz, 1H), 0.99 (td, J=12.0, 10.3, 6.1 Hz, 1H), 0.81 (s, 9H), 0.21 (dq, J=9.3, 4.5 Hz, 1H), 0.02 (d, J=15.6 Hz, 7H), −0.23 (h, J=4.8 Hz, 1H), −0.47 (dq, J=10.0, 5.0 Hz, 1H). ESI-LCMS m/z 842.1 [M+H]⁺

Preparation of compound 10b: To a solution of 9b (4.7 g, 5.6 mmol) in THF (50 mL), TBAF (9.0 mmol, 9 mL) was added at r.t. The solution was stirred 16 hours at r.t. LCMS showed 9b was consumed. The reaction was diluted with EA (200 mL) and washed with water (3×400 mL) and aqueous NaCl (200 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm, filtered to get 10b (3.4 g, 4.7 mmol, 83.9% yield) as a white solid. ¹H NMR (400 MHz, DMSO-d₆) δ 11.31 (s, 1H), 8.76 (s, 1H), 8.21 (s, 1H), 8.16-8.08 (m, 2H), 7.76-7.67 (m, 1H), 7.62 (dd, J=8.2, 6.8 Hz, 2H), 7.58-7.51 (m, 2H), 7.44-7.22 (m, 7H), 6.93 (d, J=8.7 Hz, 4H), 6.06 (d, J=6.5 Hz, 1H), 5.30 (d, J=5.8 Hz, 1H), 4.65 (td, J=5.5, 3.6 Hz, 1H), 4.38 (dd, J=6.6, 5.2 Hz, 1H), 4.02 (t, J=3.8 Hz, 1H), 3.78 (s, 6H), 3.35 (s, 3H), 2.90 (dd, J=9.0, 4.0 Hz, 1H), 1.18 (dq, J=8.3, 4.1, 3.3 Hz, 1H), 0.44 (tt, J=8.4, 4.4 Hz, 1H), 0.20 (ddt, J=13.2, 8.5, 4.6 Hz, 1H), 0.04-−0.07 (m, 1H), −0.26 (dq, J=9.9, 5.0 Hz, 1H). ESI-LCMS m/z 728.1 [M+H]⁺

Preparation of compound 305: To a solution of 10b (3.2 g, 4.4 mmol) in DCM (30 mL), DCI (500.8 mg, 3.5 mmol) and CEP[N(iPr)₂]₂ (1.9 g, 6.2 mmol) was added rapidly at rt in N₂. The solution was stirred 1 hour at r.t. LCMS showed 10b was consumed. The reaction was diluted with DCM, the solution was washed with water (3×100 ml). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm, filtered to get 305 (3.3 g, 3.6 mmol, 81.8% yield) as a white solid. ¹H NMR (400 MHz, DMSO-d₆) δ 11.31 (s, 1H), 8.75 (d, J=3.4 Hz, 1H), 8.30 (d, J=56.1 Hz, 1H), 8.17-8.10 (m, 2H), 7.78-7.68 (m, 1H), 7.68-7.53 (m, 4H), 7.48-7.21 (m, 7H), 6.93 (td, J=6.6, 3.2 Hz, 4H), 6.08 (dd, J=19.3, 6.4 Hz, 1H), 5.06-4.93 (m, 1H), 4.65-4.54 (m, 1H), 4.25 (dt, J=19.4, 3.4 Hz, 1H), 4.00-3.64 (m, 9H), 3.47-3.26 (m, 4H), 2.99 (ddd, J=9.2, 4.3, 1.9 Hz, 1H), 2.86 (ddt, J=21.7, 6.7, 4.9 Hz, 2H), 1.38-1.05 (m, 13H), 0.41 (ddq, J=13.1, 8.7, 4.2 Hz, 1H), 0.21-0.05 (m, 1H), −0.00 (dt, J=9.7, 4.9 Hz, 1H), −0.28 (dq, J=10.0, 5.1 Hz, 1H). ESI-LCMS m/z 928.4 [M+H]⁺

### Preparation of Intermediate 12a and 12b

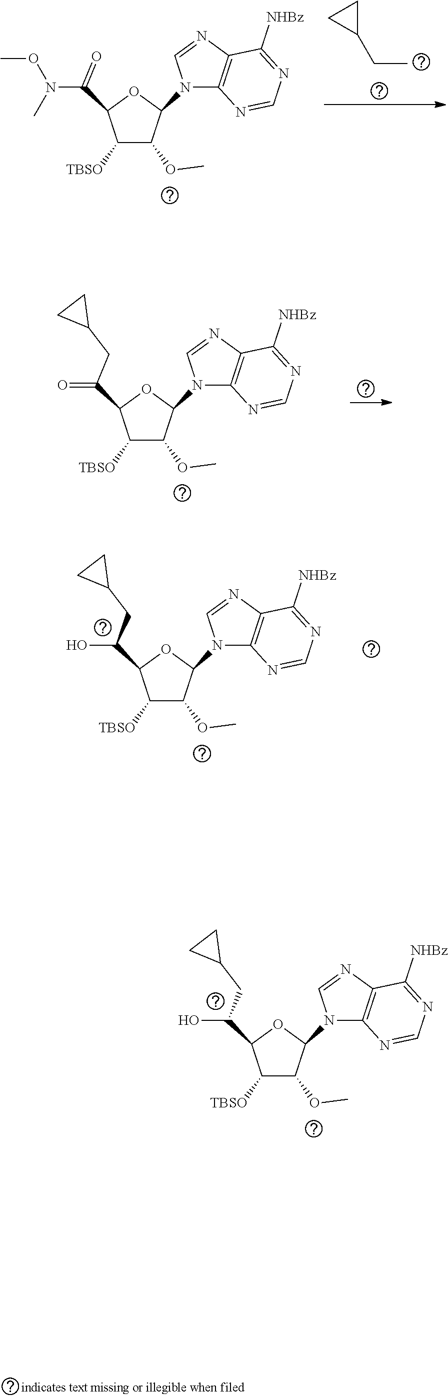

Preparation of compound 6a: Mg (5.0 g, 208.0 mmol) was activated by HCl, washed with acetone, dried by vacuum, dissolved in anhydrous THF 50 mL under N₂, catalytic amount of I₂ was added. Then the solution was heated to 70° C. for 20 min. (Bromomethyl)cyclopropane (27.0 g, 200.0 mmol, dissolved in 150 mL THF) was dropwised via a syringe and the mixture was refluxed for 1 h until Mg consumed to get 1M 6a used directly.

Preparation of compound 11: To a solution of 6 (16.7 g, 30.0 mmol) in THF (100 mL) were dropped at −18° C., The mixture was stirred for 1 h, 6a (69.00 mmol) were dropped at −18° C. The mixture was stirred for additional 1 h, TLC showed 6 was consumed completely. The reaction was diluted with 5% aqueous HCl, extracted with EA, washed with NaHCO₃, dried over anhydrous Na₂SO₄, and filtered. The filtrate was concentrated under reduced pressure to give a residue. The crude was purified by column chromatography (SiO₂, 5% MeOH in DCM) to give 11 (12.2 g, 72% yield, 98% purity) as a yellow solid. ESI-LCMS m/z 552.5 [M+H]⁺; ¹H NMR (600 MHz, DMSO-d6) δ 11.07 (s, 1H), 8.67 (s, 1H), 8.57 (s, 1H), 7.86 (d, J=7.4 Hz, 2H), 7.47 (t, J=7.4 Hz, 1H), 7.38 (t, J=7.6 Hz, 2H), 6.07 (d, J=6.5 Hz, 1H), 5.61 (m, 1H), 4.83-4.75 (m, 2H), 4.69 (m, 1H), 4.37 (m, 2H), 3.13 (d, J=10.8 Hz, 5H), 2.63 (m, 1H), 2.47 (m, 1H), 2.06 (m, 2H), 0.88 (s, 9H), 0.12 (d, J=5.7 Hz, 6H)

Preparation of compound 12a: To a solution of 11 (7.8 g, 13.9 mmol) in a round-bottom flask, HCOONa (48.8 g, 697.8 mmol) in H₂O (160 mL) was added, followed by DCM (40 mL) and (S,S) Ru-TsDPEN (91 mg, 139 μmol) . . . . The resulting two phase mixture was stirred for 24 h, TLC showed 11 was consumed completely. The reaction was diluted with DCM, washed with NaHCO₃, dried over anhydrous Na₂SO₄, and filtered. The filtrate was concentrated under reduced pressure to give a residue. The crude was purified by column chromatography (SiO₂, 5% MeOH in DCM) and Prep-SFC with the following conditions (column 3*15 mm, mobile phase, 10% CO₂ and alcohols; detector UV) to give 12a (4.8 g, 60% yield, 98% purity) as a yellow solid. ESI-LCMS m/z 554.5 [M+H]⁺; 1H NMR (400 MHz, CD₃CN) δ 9.41 (s, 1H), 8.46 (s, 1H), 8.22 (s, 1H), 7.85 (d, J=7.4 Hz, 2H), 7.47 (t, J=7.4 Hz, 1H), 7.37 (t, J=7.6 Hz, 2H), 5.90 (d, J=6.5 Hz, 1H), 5.71 (m, 1H), 4.92-4.79 (m, 2H), 4.71 (d, J=9.3 Hz, 1H), 4.44 (m, 1H), 4.33 (m, 1H), 3.89 (t, J=2.1 Hz, 1H), 3.58 (m, 1H), 3.14 (s, 3H), 2.05 (m, 2H), 1.47 (m, 2H), 0.80 (s, 9H), 0.00 (d, J=1.5 Hz, 6H)

Preparation of compound 12b: To a solution of 11 (5.4 g, 8.7 mmol) in a round-bottom flask, CHOONa (31.3 g, 435.0 mmol) in H₂O (100 mL) was added, followed by BA (25 mL) and (R,R) Ru-TsDPEN (55 mg, 87 μmol). The resulting two phase mixture was stirred for 24 h, TLC showed 11 was consumed completely. The reaction was diluted with EA, washed with NaHCO₃, dried over anhydrous Na₂SO₄, and filtered. The filtrate was concentrated under reduced pressure to give a residue. The crude was purified by column chromatography (SiO₂, 5% MeOH in DCM) to give 12b (4.5 g, 87% yield, 98% purity) as a yellow solid. ESI-LCMS m/z 554.5 [M+H]⁺; ¹H NMR (400 MHz, CD₃CN) δ 9.29 (s, 1H), 8.46 (3, 1H), 8.13 (8, 1H), 7.82 (d, J=7.4 Hz, 2H), 7.47 (t, J=7.4 Hz, 1H), 7.37 (1, J=7.6 Hz, 2H), 5.83 (d, J=7.4 Hz, 1H), 5.71 (m, 1H), 5.18 (s, 1H), 4.92-4.79 (m, 2H), 4.44 (m, 1H), 4.37 (m, 1H), 3.85 (m, 1H), 3.66 (m, 1H), 3.08 (s, 3H), 2.15 (m, 1H), 1.96 (m, 1H), 1.41 (m, 2H), 0.79 (s, 9H), 0.01 (d, J=1.2 Hz, 6H)

### Preparation of Compound 304

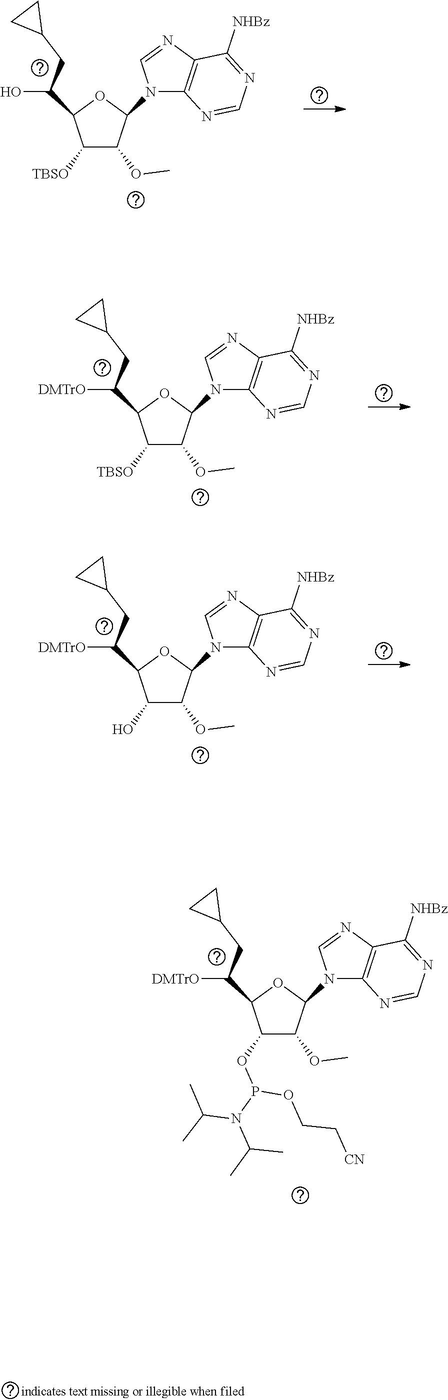

Preparation of compound 13a: To a solution of 12a (4.2 g, 7.6 mmol) in DCM (42 mL), collidine (2.7 g, 22.7 mmol) was added at r.t under N₂. Then DMTrCl (5.1 g, 15.1 mmol) and AgNO₃ (1.9 g, 11.4 mmol) was added at r.t. The solution was stirred 4 hours at r.t. LCMS showed 12a was consumed. The solution was quenched by addition of water (20 mL) and diluted with DCM (200 mL), then filter to diatomite. The solution was washed with water (2×500 mL), and aqueous NaCl (200 ml). The organic phase dried over anhydrous Na₂SO₄, concentrated by rotary evaporator to give crude. The crude was purified by silica gel; mobile phase, PE/EA=10/1 increasing to PE/EA=1/1, the eluted product was collected at PE/EA=2/1 to give 13a (5.2 g, 6.1 mmol, 80.3% yield) as white solid. ¹H NMR (400 MHz, DMSO-d₆) δ 11.25 (s, 1H), 8.71 (d, J=14.2 Hz, 2H), 8.07-8.00 (m, 2H), 7.66-7.42 (m, 5H), 7.39-7.31 (m, 4H), 7.17 (dt, J=28.7, 7.4 Hz, 3H), 6.81 (dd, J=8.9, 7.4 Hz, 4H), 6.08 (d, J=6.7 Hz, 1H), 5.39-5.15 (m, 1H), 5.06 (dd, J=6.8, 4.9 Hz, 1H), 4.72-4.63 (m, 2H), 4.41 (dd, J=4.9, 2.8 Hz, 1H), 4.07-3.88 (m, 1H), 3.68 (d, J=1.6 Hz, 6H), 3.25 (s, 3H), 1.76 (d, J=5.7 Hz, 1H), 1.57-1.02 (m, 3H), 0.81 (s, 9H), 0.00 (s, 3H), −0.11 (s, 3H). ESI-LCMS m/z 856.1 [M+H]⁺

Preparation of compound 14a: To a solution of 13a (5 g, 5.8 mmol) in THF (50 mL), TBAF (9 mmol, 9 mL) was added at r.t in N₂. The solution was stirred 16 hours at r.t. LCMS showed 13a was consumed. The reaction was diluted with EA (200 mL) and washed with water (3×400 mL) and aqueous NaCl (200 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 25 min, the eluted product was collected at CH₃CN/H₂O (0.5% NH₄HCO₃)=1/1; Detector, UV 254 nm, filtered to get 14a (3.6 g, 4.8 mmol, 82.7% yield) as a white solid. ¹H NMR (400 MHz, DMSO-d₆): δ 1.22-1.43 (m, 2H), δ 1.58-1.80 (m, 2H), 3.36-3.42 (m, 4H), 3.72 (d, J=3.16 Hz, 4H), 3.32 (s, 6H), 4.01 (t, J=4.2 Hz, 1H), 4.41-4.46 (m, 1H), 4.71-4.79 (m, 3H), 5.23 (d, J=6.21 Hz, 1H), δ 5.35-5.46 (m, 1H), 6.06 (d, J=6.11 Hz, 1H), 6.78-6.84 (m, 4H), 7.14-7.32 (m, 10H), 7.45 (d, J=8.44 Hz, 2H), 8.05 (d, J=7.94 Hz, 2H), 8.65 (s, 1H), 8.72 (s, 1H), 11.24 (s, 1H) ESI-LCMS m/z 742.1 [M+H]⁺

Preparation of compound 304: To a solution of 14a (3.4 g, 4.6 mmol) in DCM (30 mL), DCI (461.4 mg, 3.9 mmol) and CEP[N(iPr)₂]₂ (1.8 g, 6.1 mmol) was added rapidly at r.t under N₂. The solution was stirred 1 hour at r.t. LCMS showed 14a was consumed. The reaction was diluted with DCM (200 mL), the solution was washed with water (3×100 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm, filtered to get 304 (3.5 g, 3.7 mmol, 80.0% yield) as a white solid. ¹H NMR (400 MHz, DMSO-d₆): δ 1.02-1.56 (m, 16H), 61.74-1.78 (m, 1H), 2.39-2.62 (m, 1H), 2.78 (t, J=5.28 Hz, 1H), 3.32-3.34 (m, 3H), 3.56-3.82 (m, 10H), 4.15-4.20 (m, 1H), 4.56-4.74 (m, 3H), 5.16-5.40 (m, 2H), 6.13 (t, J=7.26 Hz, 1H), 6.80-6.86 (m, 4H), 6.80-6.86 (m, 4H), 7.17-7.25 (m, 3H), 7.33-7.39 (m, 4H), 7.46-7.66 (m, 5H), 8.06-8.09 (m, 2H), 8.69-8.76 (m, 2H), 11.28 (d, J=5.45 Hz, 1H). ³¹P NMR (400 MHz, DMSO-d₆): δ 149.71 (s, P), δ 149.92 (s, 9P). ESI-LCMS m/z 942.4 [M+H]⁺

### Preparation of Compound 303

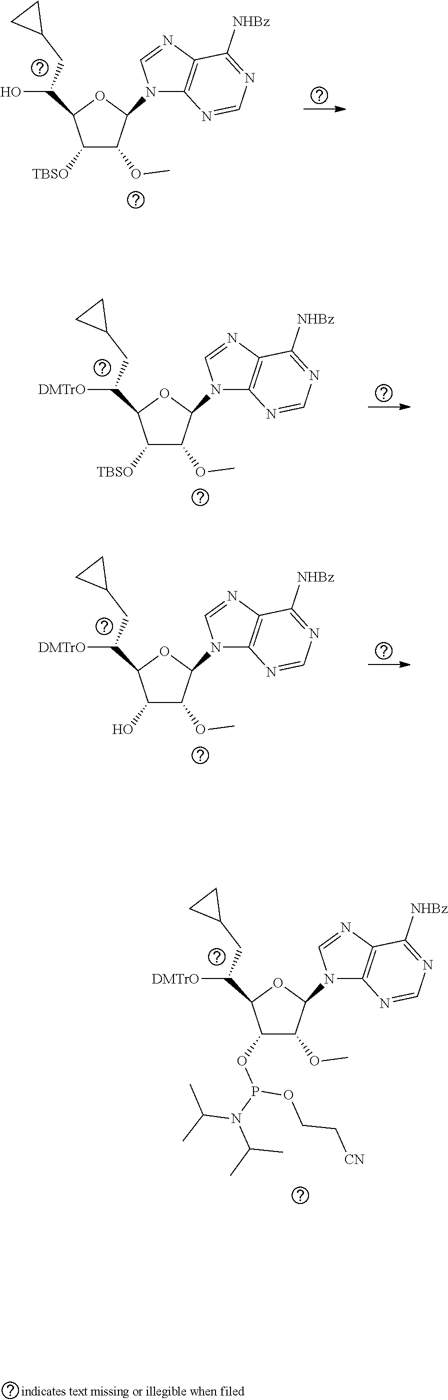

Preparation of compound 13b: To a solution of 12b (3.6 g, 6.5 mmol) in DCM (40 mL), collidine (2.4 g, 19.5 mmol) was added at r.t under N₂. Then DMTrCl (4.4 g, 13.0 mmol) and AgNO₃ (1.6 g, 9.7 mmol) was added at r.t. The solution was stirred 4 hours at r.t. LCMS showed 12b was consumed. The solution was quenched by addition of water (20 mL) and diluted with DCM (200 mL), then filter to diatomite. The solution was washed with water (2×500 mL), and aqueous NaCl (200 ml). The organic phase dried over anhydrous Na₂SO₄, concentrated by rotary evaporator to give crude. The crude was purified by silica gel; mobile phase, PE/EA=10/1 increasing to PE/EA=1/1, the eluted product was collected at PE/EA=2/1 to give 13b (4.7 g, 5.5 mmol, 84.6% yield) as white solid. ¹H NMR. (400 MHz, DMSO-d₆) δ 11.14 (s, 1H), 8.68 (s, 1H), 8.53 (s, 1H), 8.02-7.93 (m, 2H), 7.61-7.52 (m, 1H), 7.54-7.34 (m, 4H), 7.31-7.09 (m, 7H), 6.86-6.74 (m, 4H), 5.90 (d, J=5.8 Hz, 1H), 5.40 (ddt, J=16.0, 11.2, 6.6 Hz, 1H), 4.74-4.63 (m, 2H), 4.36 (t, J=4.1 Hz, 1H), 4.19 (dd, J=5.8, 4.6 Hz, 1H), 4.00-3.88 (m, 2H), 3.80 (q, J=5.0 Hz, 1H), 3.64 (s, 3H), 3.25 (s, 1H), 3.02 (s, 3H), 1.95 (d, J=13.7 Hz, 1H), 1.77 (s, 1H), 1.28 (s, 1H), 1.15 (dq, J=6.6, 4.2, 3.5 Hz, 1H), 0.83 (s, 9H), 0.06 (s, 3H), 0.00 (s, 3H), ESI-LCMS m/z 856.1 [M+H]⁺

Preparation of compound 14b: To a solution of 13b (4.5 g, 5.3 mmol) in THE (50 mL), TBAF (7.0 mmol, 7 mL) was added at r.t. The solution was stirred 16 hours at r.t. LCMS showed 13b was consumed. The reaction was diluted with EA (200 mL), washed with water (3×400 mL) and aqueous NaCl (200 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=1/0 within 25 min, the eluted product was collected at CH₃CN/H₂O (0.5% NHAHCO₃)=1/1; Detector, UV 254 nm, filtered to get 14b (3.3 g, 4.4 mmol, 83.0% yield) as a white solid. ¹H NMR (400 MHz, DMSO-d₆) δ 11.25 (s, 1H), 8.73 (s, 1H), 8.52 (s, 1H), 8.09-8.02 (m, 2H), 7.70-7.60 (m, 1H), 7.56 (dd, J=8.3, 6.9 Hz, 2H), 7.51-7.43 (m, 2H), 7.39-7.09 (m, 7H), 6.89 (dd, J=10.4, 8.0 Hz, 4H), 5.99 (d, J=6.3 Hz, 1H), 5.48 (ddt, J=16.0, 11.0, 6.6 Hz, 1H), 5.30 (d, J=5.7 Hz, 1H), 4.82-4.72 (m, 2H), 4.43-4.29 (m, 2H), 4.02-3.97 (m, 1H), 3.73 (d, J=2.7 Hz, 7H), 3.23 (s, 3H), 1.98-1.91 (m, 1H), 1.90-1.77 (m, 1H), 1.44-1.21 (m, 2H). ESI-LCMS m/z 742.1 [M+H]⁺

Preparation of compound 303: To a solution of 14b (3.0 g, 4.0 mmol) in DCM (30 mL), DCI (405.9 mg, 3.4 mmol) and CEP[N(Pr)₂]₂ (1.8 g, 6.1 mmol) was added rapidly at rt in N₂. The solution was stirred 1 hour at r.t. LCMS showed 14b was consumed. The reaction was diluted with DCM (200 mL), the solution was washed with water (3×100 mL). The organic phase dried over anhydrous Na₂SO₄, concentrated by rotary evaporator to give crude. The crude was purified by Flash-Prep-HPLC with the following conditions (IntelFlash-1): Column, C18 silica gel; mobile phase, CH₃CN/H₂O (0.5% NHAHCO₃)=1/1 increasing to CH₃CN/H₂O (0.5% NH₄HCO₃)=1/0 within 25 min, the eluted product was collected at CH₃CN/H₂O (0.5% NH₄HCO₃)=4/1; Detector, UV 254 nm, filtered to give 303 (3.1 g, 3.3 mmol, 82.5% yield) as a white solid. ¹H NMR (400 MHz, DMSO-d₆) δ 11.23 (s, 1H), 8.71 (d, J=5.6 Hz, 1H), 8.09-8.01 (m, 2H), 7.69-7.60 (m, 1H), 7.52 (ddd, J=27.2, 8.4, 6.8 Hz, 4H), 7.41-7.16 (m, 7H), 6.96-6.81 (m, 4H), 6.03 (dd, J=24.0, 5.7 Hz, 1H), 5.54-5.38 (m, 1H), 4.81-4.59 (m, 3H), 4.50-4.40 (m, 1H), 4.26-4.12 (m, 1H), 3.95-3.56 (m, 11H), 3.22 (d, J=35.4 Hz, 3H), 2.78 (ddt, J=12.5, 10.5, 3.8 Hz, 2H), 2.13-1.70 (m, 2H), 1.45-1.09 (m, 14H). ESI-LCMS m/z 942.4 [M+H]⁺

### Example 33—Preparation of Compound 309

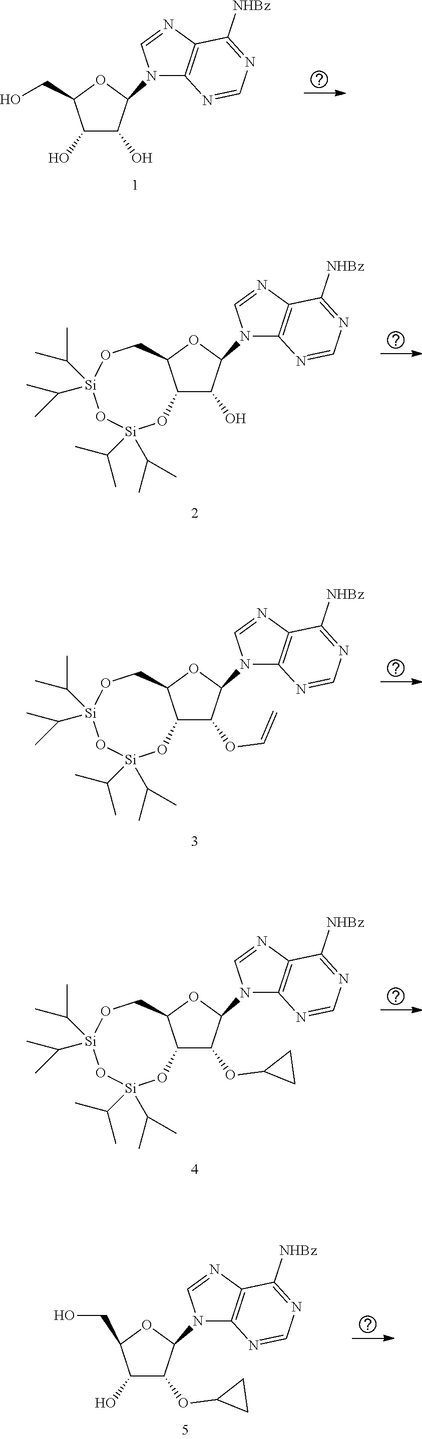

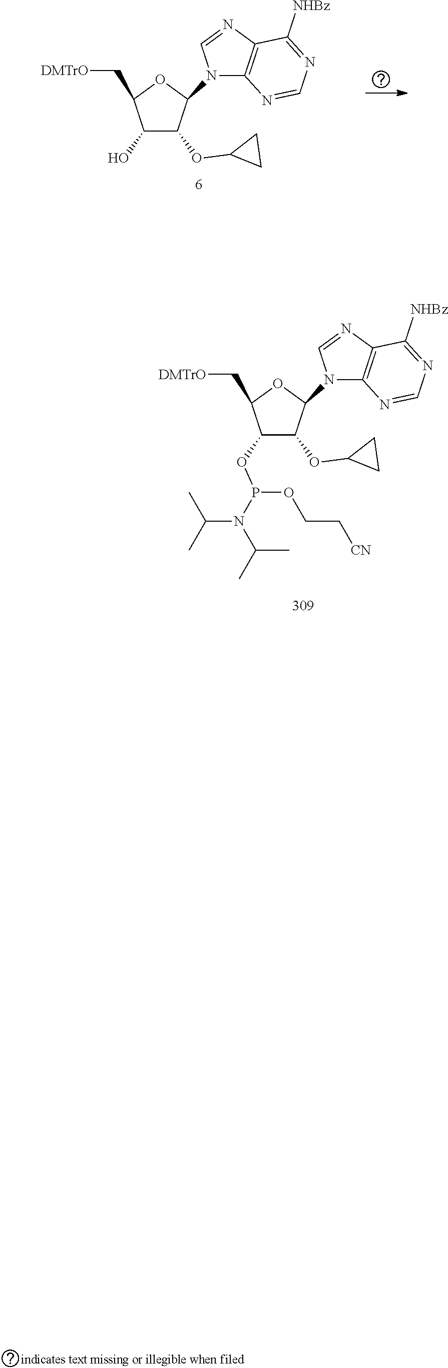

Preparation of 2: To a solution of 1 (30 g, 80.78 mmol) in DMF (300 mL). Then the mixture was added Imidazole (13.7 g, 201.97 mmol), TiPSCl₂ (33.2 g, 105.01 mmol) under N₂ atmosphere. The mixture was stirred at r.t. for 4 h. Then the solution was diluted with EA, washed with water twice. The solvent was concentrated under reduced pressure to give the crude. The crude was purified by silica gel; mobile phase, PE/EA=50/1 increasing to PE/EA=1/1, the eluted product was collected at PE/EA=2/1; filtered to get 2 (43 g, 69.91 mmol, 86.5% yield) as a solid. ESI-LCMS: m/z 615.2 [M+H]⁺. ¹H NMR (400 MHz, DMSO-d6) § 11.23 (s, 1H), 8.68 (s, 1H), 8.56 (s, 1H), 8.06 (d, J=7.6 Hz, 2H), 7.65 (t, J=7.4 Hz, 1H), 7.56 (t, J=7.6 Hz, 2H), 6.03 (s, 1H), 5.69 (d, J=4.6 Hz, 1H), 4.85 (dd, J=8.2, 5.2 Hz, 1H), 4.67 (t, J=5.1 Hz, 1H), 4.24-3.83 (m, 4H), 3.35 (s, OH), 2.51 (p, J=1.8 Hz, 1H), 1.99 (s, 1H), 1.28-0.79 (m, 26H).

Preparation of 3: To a solution of 2 (43 g, 69.91 mmol) in DCM (400 mL). The solution was added PPTS (3.5 g, 13.98 mmol), EVE (65.4 g, 908.83 mmol). The mixture was stirred at 25° C. for 16 h. LCMS showed 2 was consumed completely. The solvent was washed with water twice and filtered to get the crude 2-1. ESI-LCMS: m/z 686 [M+H]⁺.

To a solution of 2-1 in DCM (300 mL). The solution was added TEA (91.8 g, 908.83 mmol) and was cooled to 0° C. The mixture was added TMSOTf (155.4 g, 699.1 mmol) drop wise at 0° C. under argon atmosphere. The mixture was stirred at 25° C. for 16 h. LCMS showed 2-1 was consumed completely. The solvent was washed with water twice and filtered to get the crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 3 (33 g, 51.56 mmol, 73.7% yield) as a solid. ESI-LCMS: m/z 640 [M+H]⁺.

Preparation of 4: To a solution of 1M Et₂Zn (93.8 ml, 93.75 mmol) and DIM (25.1 g, 93.75 mmol) in DCM (225 mL) was stirred at 0° C. for 10 min. The mixture was added 3 (15 g, 23.4 mmol) drop wise under argon atmosphere. The mixture was stirred at 25° C. for 6 h. NH₄Cl (aq.) was added to the mixture. Then filtered and the solution diluted with EA. The organic layer was washed with water and brine. The organic layer was concentrated under reduced pressure and filtered to get a crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 4 (3.4 g, 5.19 mmol, 22.2% yield) as a solid. ESI-LCMS: m/z 654.7 [M+H]⁺; ¹H NMR (400 MHz, DMSO-d6) δ 11.24 (s, 1H), 8.68 (s, 1H), 8.52 (s, 1H), 8.09-8.01 (m, 2H), 7.70-7.61 (m, 1H), 7.56 (t, J=7.6 Hz, 2H), 6.06 (s, 1H), 5.01 (dd, J=9.0, 5.1 Hz, 1H), 4.67 (d, J=5.1 Hz, 1H), 4.11-3.91 (m, 3H), 3.83 (tt, J=5.9, 2.9 Hz, 1H), 1.40 (s, 1H), 1.30-0.84 (m, 28H), 0.70-0.20 (m, 4H).

Preparation of 5: To a solution of 4 (3.4 g, 5.19 mmol) in THF (30 mL) and added 1M TBAF (7.8 ml, 7.78 mmol). The mixture was stirred at r.t. for 6 h. LCMS showed 4 was consumed completely. H₂O was added to the mixture. Then filtered and the solution diluted with EA. The organic layer was washed with NaHCO₃ (aq.) and brine. The solvent was concentrated under reduced pressure and filtered to get a crude. The crude was purified by silica gel; mobile phase, DCM/MeOH=100/1 increasing to DCM/MeOH=20/1, the eluted product was collected at DCM/MeOH=30/1; filtered to get 5 (2 g, 4.85 mmol, 93.4% yield) as a solid. ESI-LCMS: m/z 412.3 [M+H]⁺.

Preparation of 6: To a stirred mixture of 5 (2 g, 4.85 mmol) in pyridine (20 mL) was added DMTrCl (1.8 g, 5.33 mmol) at room temperature under N₂ atmosphere. The resulting mixture was stirred for 3 h at room temperature under argon atmosphere. The reaction was quenched by the addition of sat. NaHCO₃ (aq.). The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE (0.5% TEA)/EtOAc (2:3) to afford 6 (2.7 g, 3.79 mmol, 78.2%) as an off-white solid.

ESI-LCMS: m/z 714.3 [M+H]⁺; ¹H NMR (400 MHz, DMSO-d₆) δ 11.23 (s, 1H), 8.65 (d, J=23.3 Hz, 2H), 8.18-7.93 (m, 2H), 7.75-7.47 (m, 3H), 7.47-7.06 (m, 9H), 6.96-6.69 (m, 4H), 6.18 (d, J=5.4 Hz, 1H), 5.40 (dd, J=5.9, 1.8 Hz, 1H), 4.86 (t, J=5.0 Hz, 1H), 4.49 (q, J=5.1 Hz, 1H), 4.12 (q, J=4.4 Hz, 1H), 3.72 (s, 6H), 3.58-3.48 (m, 1H), 3.31-3.19 (m, 2H), 0.65-0.33 (m, 4H).

Preparation of 309: To a solution of 6 (2.7 g, 3.79 mmol) in DCM (30 mL) was added DCI (381 mg, 3.22 mmol) and CEP[N(iPr)₂]₂ (1.4 g, 4.55 mmol) under N₂. The mixture was stirred at 20° C. for 2.5 h. LCMS showed 6 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 309 (3.2 g, 3.40 mmol, 89.7% yield) as a white solid. ESI-LCMS: m/z 914.0 [M+H]⁺; ¹H NMR (400 MHz, DMSO-d6) δ 11.25 (s, 1H), 8.71-8.61 (m, 2H), 8.09-8.02 (m, 2H), 7.70-7.46 (m, 3H), 7.46-7.07 (m, 9H), 6.84 (ddd, J=9.2, 6.5, 3.3 Hz, 4H), 6.21 (ddd, J=19.3, 5.5, 1.8 Hz, 1H), 5.15-5.06 (m, 1H), 4.78 (dt, J=9.7, 4.5 Hz, 1H), 4.26 (dq, J=25.4, 4.4 Hz, 1H), 3.96-3.47 (m, 9H), 3.47-3.16 (m, 3H), 2.81 (t, J=5.9 Hz, 1H), 2.64 (t, J=5.9 Hz, 1H), 1.38-0.93 (m, 13H), 0.70-0.29 (m, 4H); ³¹P NMR (162 MHz, Acetonitrile-d3) δ 149.60.

### Example 34—Preparation of R-Ganciclovir & S-Ganciclovir Precursor

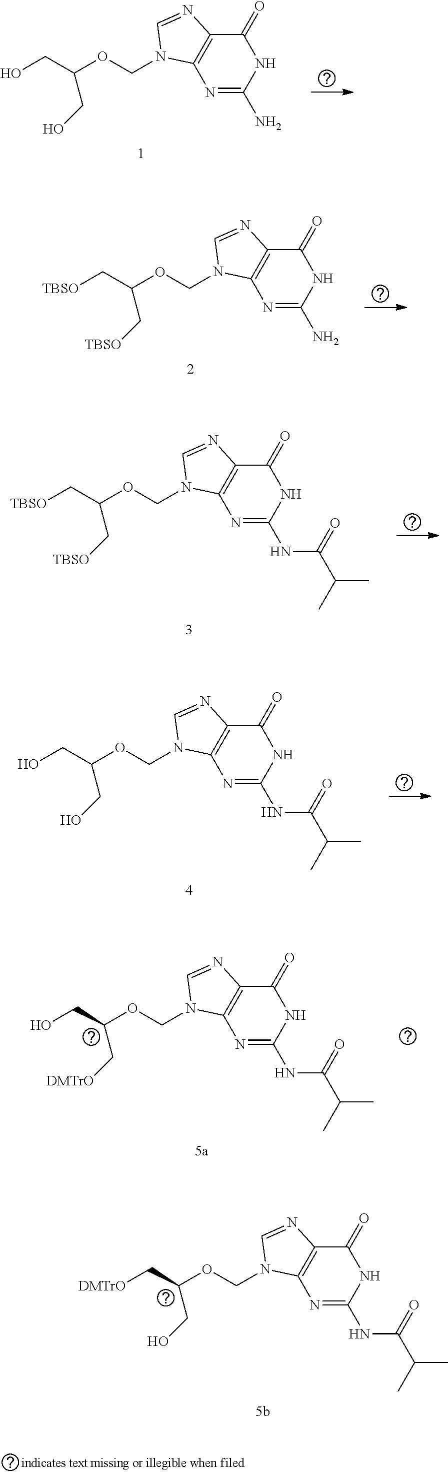

Preparation of 2: To a solution of SM-1 (20.0 g, 78.4 mmol) in DMF (160 mL), imidazole (18.7 g, 274.3 mmol) and TBSCl (29.5 g, 195.9 mmol) was added at r.t. The solution was stirred at r.t under N₂ for 16 h. LC-MS showed 1 was consumed completely. The reaction was added ACN (320 ml), the mixture was purified by slurry and washed with ACN to give 2 (35.2 g, 68.7 mmol, 87.7% yield, 95% purity) as a white solid. ESI-LCMS: m/z 484.3 [M+H]⁺.

Preparation of 3: To a solution of 2 (30.0 g, 62.1 mmol) in pyridine (300 mL) was dropped iBuCl (9.2 g, 86.9 mmol) at 0° C. The resulting mixture was stirred for 3 h at r.t, TLC showed 2 was consumed completely. The reaction was added NaHCO₃ solution, then extracted with EA. Then the organic phase was washed once with saturated brine and dried over by Na₂SO₄. Then the solution was concentrated under reduced pressure. The residue was purified twice by slurry and washed with PE to give 3 (31.0 g, 55.9 mmol, 90.1% yield, 95% purity) as a white solid. ESI-LCMS: m/z 554.3 [M+H]⁺.

Preparation of 4: To a solution of 3 (29.0 g, 52.4 mmol) in ACN (300 mL) was added 3HF·TEA (42.2 g, 262.0 mmol). The resulting mixture was stirred for 3 h at r.t, TLC showed 3 was consumed completely. The reaction was cooled to −78° C. and filtered. This resulted in 4 (15.7 g, 48.3 mmol, 92.2% yield, 98% purity) as a white solid. ESI-LCMS: m/z 326.0 [M+H]⁺ ¹H NMR (400 MHz, DMSO-d₆) δ 11.82 (s, 2H), 8.12 (s, 1H), 5.58 (s, 2H), 4.68 (6, 2H), 3.44 (ddd, J=55.3, 44.7, 5.7 Hz, 4H), 2.86 (dq, J=41.1, 7.0 Hz, 2H), 1.13 (d, J=6.9 Hz, 6H).

Preparation of 5 and 5a: To a solution of 4 (6.0 g, 18.4 mmol) in pyridine (60 mL) was added DMTrCl (6.2 g, 18.4 mmol). The resulting mixture was stirred at r.t for 16 b. TLC and LC-MS showed 4 was consumed completely. The reaction was added NaHCO₃ solution, then extracted with EA. Then the organic phase was washed once with saturated brine and dried over by Na₂SO₄. Then the solution was concentrated under reduced pressure. The residue was purified by silica gel column chromatograph (eluent, DCM/MeOH=30:1). The residue was purified by prep-SFC. This resulted in 5 (2.2 g, 3.5 mmol, 19.0% yield, 98% purity) and 5a (2.1 g, 3.3 mmol, 17.9% yield, 98% purity) as a white solid. ESI-LCMS: m/z 628.3 [M+H]⁺.

5a ¹H NMR (400 MHz, DMSO-d₆) δ 12.11 (s, 1H), 11.86 (s, 1H), 8.26 (s, 1H), 7.31-6.97 (m, 9H), 6.83 (dd, J=9.0, 2.6 Hz, 4H), 5.66 (q, J=11.5 Hz, 2H), 4.76 (t, J=5.2 Hz, 1H), 3.93 (b, J=6.2, 5.7 Hz, 1H), 3.73 (s, 6H), 3.04-2.71 (m, 3H), 1.12 (dd, J=6.9, 2.6 Hz, 6H).

5b ¹H NMR (400 MHz, DMSO-d₆) δ 12.11 (s, 1H), 11.86 (s, 1H), 8.26 (s, 1H), 7.31-6.97 (m, 9H), 6.83 (dd, J=9.0, 2.6 Hz, 4H), 5.66 (q, J=11.5 Hz, 2H), 4.76 (t, J=5.2 Hz, 1H), 3.93 (h, J=6.2, 5.7 Hz, 1H), 3.73 (s, 6H), 3.04-2.71 (m, 3H), 1.12 (dd, J=6.9, 2.6 Hz, 6H).

### Example 35—Preparation of Compound 310

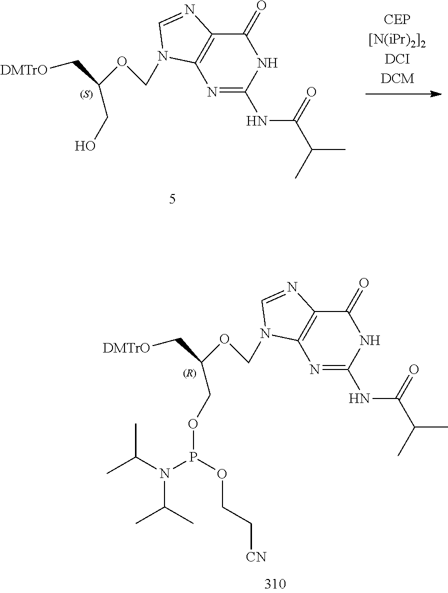

Preparation of 310: To a solution of 5 (2.6 g, 4.1 mmol) in DCM (26 mL) was added DCI (141 mg, 2.5 mmol) and CEP[N(iPr)₂]₂ (593 mg, 4.9 mmol) under N₂. The mixture was stirred at r.t for 1 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated to give the crude. The crude was purified by Flash-Prep-HPLC with the following conditions (IntelFlash-1): Column, C18 silica gel, mobile phase, CH₃CN/H₂O (0.05% NH₄HCO₃)=3/2 increasing to CH₃CN/H₂O (0.05% NH₄HCO₃)=1/0 within 20 min, the eluted product was collected at CH₃CN/H₂O (0.05% NH₄HCO₃)=1/0; Detector, UV 254 nm. This resulted in 310 (2.4 g, 2.9 mmol, 70.7% yield, 99% purity) as a white solid. ESI-LCMS: m/z 828.4 [M+H]⁺. 1H NMR. (400 MHz, DMSO-d₆) δ 11.95 (d, J=99.7 Hz, 2H), 8.25 (s, 1H), 7.30-7.13 (m, SH), 7.12-7.02 (m, 4H), 6.82 (dt, J=9.1, 2.7 Hz, 4H), 5.74-5.44 (m, 2H), 4.20-3.97 (m, 1H), 3.87-3.36 (m, 12H), 3.08-2.86 (m, 2H), 2.80 (hept, J=6.7 Hz, 1H), 2.68 (q, J=5.9 Hz, 2H), 1.16-1.02 (m, 12H), 0.96 (dd, J=6.7, 3.0 Hz, 6H), ³¹P NMR (162 MHz, DMSO-d₆) δ 147.29 (d, J=5.8 Hz).

### Example 36—Preparation of Compound 311

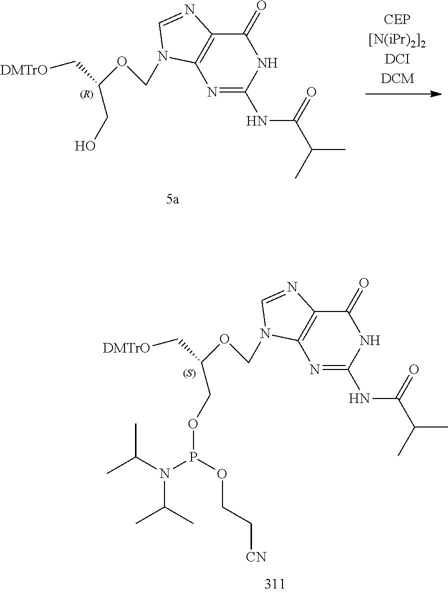

Preparation of 311: To a solution of 5a (2.0 g, 3.2 mmol) in DCM (20 mL) was added DCI (248 mg, 1.9 mmol) and CEP[N(iPr)₂]₂ (1.1 g, 3.8 mmol) under N₂. The mixture was stirred at r.t for 1 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated to give the crude. The crude was purified by Flash-Prep-HPLC with the following conditions (IntelFlash-1): Column, C18 silica gel; mobile phase, CH₃CN/H₂O (0.05% NH₄HCO₃)=3/2 increasing to CH₃CN/H₂O (0.05% NH₄HCO₃)=1/0 within 20 min, the eluted product was collected at CH₃CN/H₂O (0.05% NH₄HCO₃)=1/0; Detector, UV 254 nm. This resulted in 311 (1.5 g, 1.8 mmol, 56.2% yield, 99% purity) as a white solid. ESI-LCMS: m/z 828.4 [M+H]⁺. 1H NMR (400 MHz, DMSO-d₆) δ 12.07 (s, 1H), 11.82 (s, 1H), 8.25 (s, 1H), 7.31-7.14 (m, 5H), 7.12-7.00 (m, 4H), 6.82 (dt, J=9.1, 2.7 Hz, 4H), 5.75-5.51 (m, 2H), 4.06 (ddq, J=12.2, 8.4, 5.0 Hz, 1H), 3.81-3.34 (m, 12H), 3.08-2.86 (m, 2H), 2.80 (hept, J=6.8 Hz, 1H), 2.68 (q, J=5.9 Hz, 2H), 1.17-1.03 (m, 12H), 0.96 (dd, J=6.8, 3.0 Hz, 6H). ³¹P NMR (162 MHz, DMSO-d₆) δ 147.29 (d, J=5.9 Hz).

### Example 37—Preparation of R-Penciclovir & S-Penciclovir Precursor

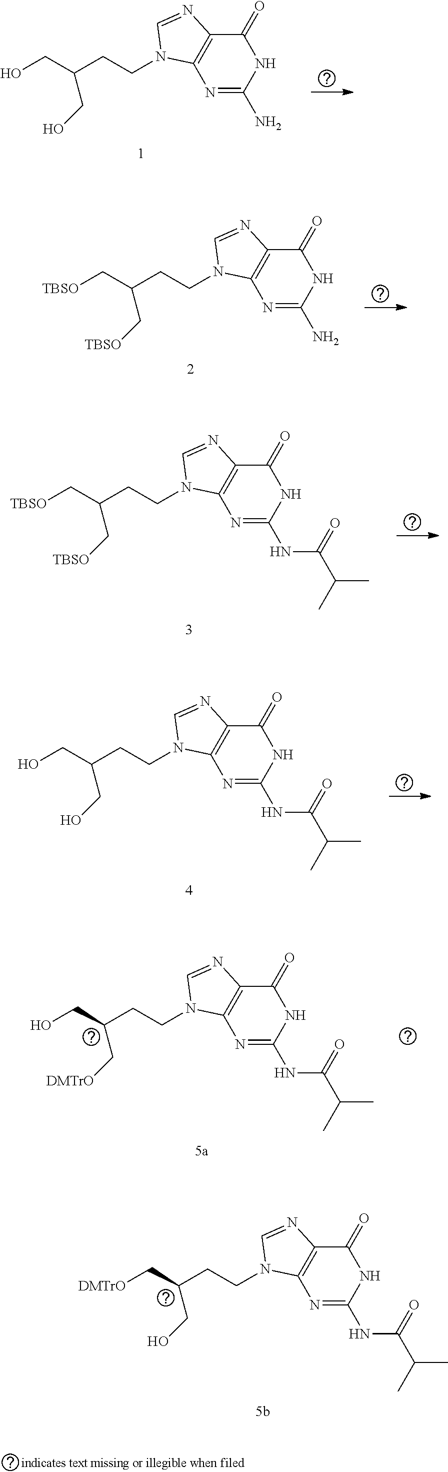

Preparation of 2: To a solution of 1 (25.0 g, 98.7 mmol) in DMF (250 mL) was added TBSCl (20.3 g, 246 mmol), imidazole (33.6 g, 493 mmol). The resulting mixture was stirred at r.t for 16 h, TLC showed 1 was consumed completely. The reaction was added NaHCO₃ solution, then extracted with EA. Then the organic phase was washed once with saturated brine and dried over by Na₂SO₄. Then the solution was concentrated under reduced pressure. The product was purified by slurry and washed with ACN to give 2 (25 g, 51% yield, 98.0% purity) as a white solid. ESI-LCMS: m/z 482.5 [M+H]⁺.

Preparation of 3: To a solution of 2 (25.0 g, 51.8 mmol) in pyridine (250 mL) was dropped iBuCl (4.8 g, 45.2 mmol) at 0° C. The resulting mixture was stirred at r.t for 3 h, TLC showed 2 was consumed completely. The reaction was added NaHCO₃ solution, then extracted with EA. Then the organic phase was washed once with saturated brine and dried over by Na₂SO₄. Then the solution was concentrated under reduced pressure. The residue was purified twice by slurry and washed with PE to give 3 (25 g, 85% yield, 95.0% purity) as a white solid. ESI-LCMS: m/z 552.5 [M+H]⁺.

Preparation of 4: To a solution of 3 (25.0 g, 46.4 mmol) in ACN (250 mL) was added HF·TEA (22.4 g, 139.4 mmol). The resulting mixture was stirred at r.t for 3 h, TLC showed 3 was consumed completely. The reaction was filtered. This resulted in 4 (8.0 g, 54.0% yield, 98.0% purity) as a white solid. ESI-LCMS: m/z 324.1 [M+H]⁺.

Preparation of 5a and 5b: To a solution of 4 (6 g, 19.4 mmol) in pyridine (60 mL) was added DMTrCl (6.5 g, 19.4 mmol). The resulting mixture was stirred at r.t for 16 h. TLC and LC-MS showed 4 was consumed completely. The reaction was added NaHCO₃ solution, then extracted with EA. Then the organic phase was washed once with saturated brine and dried over by Na₂SO₄. Then the solution was concentrated under reduced pressure. The residue was purified by silica gel column chromatograph (eluent, DCM/MeOH=30:1). The residue was purified by prep-SFC. This resulted in 5b (2.2 g, 98% purity) and 5a (2.1 g, 98% purity) as a white solid. ESI-LCMS: m/z 626.1 [M+H]⁺.

5a ¹H NMR (600 MHz, DMSO-d₆) δ 12.09 (s, 1H), 11.66 (s, 1H), 7.92 (s, 1H), 7.45-7.03 (m, 9H), 6.86 (d, J=8.4 Hz, 4H), 4.53 (t, J=5.1 Hz, 1H), 4.07 (hept. J=6.3 Hz, 2H), 3.48 (dh, J=21.1, 5.1 Hz, 2H), 3.10-2.71 (m, 3H), 2.01-1.55 (m, 3H), 1.21-1.04 (m, 6H).

5b ¹H NMR (600 MHz, DMSO-d₆) δ 12.09 (s, 1H), 11.66 (s, 1H), 7.92 (s, 1H), 7.45-7.03 (m, 9H), 6.86 (d, J=8.4 Hz, 4H), 4.53 (t, J=5.1 Hz, 1H), 4.07 (hept, J=6.3 Hz, 2H), 3.48 (dh, J=21.1, 5.1 Hz, 2H), 3.10-2.71 (m, 3H), 2.01-1.55 (m, 3H), 1.21-1.04 (m, 6H).

### Example 38—Preparation of Compound 312

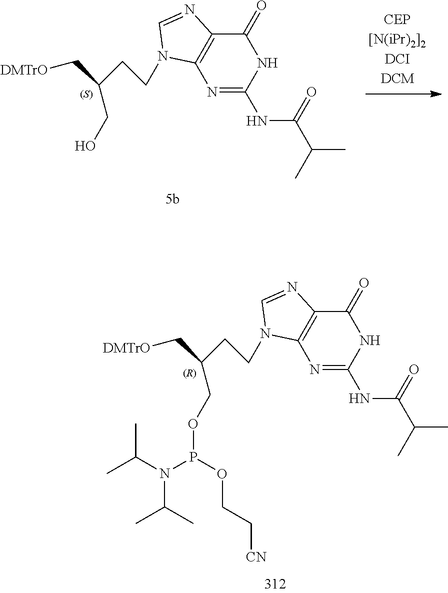

Preparation of 312: To a solution of 5b (1.8 g, 2.9 mmol) in DCM (18 mL) was added DCI (200 mg, 1.7 mmol) and CEP[N(iPr)₂]₂ (1.0 g, 3.5 mmol) under N₂. The mixture was stirred at r.t for 1 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated. The residue was purified by silica gel column chromatograph (eluent, PE/BA=1:1). The residue was purified by Flash-Prep-HPLC with the following conditions (IntelFlash-1): Column, C18 silica gel; mobile phase, CH₃CN/H—O (0.05% NH₄HCO₃)=1/1 increasing to CH₃CN/H₂O (0.05% NHAHCO₃)=1/0 within 25 min, the eluted product was collected at CH₃CN/H₂O (0.05% NHAHCO₃)=1/0; Detector, UV 254 nm. This resulted in 312 (500 mg, 0.6 mmol, 20.7% yield, 99.0% purity) as a white solid. ESI-LCMS: m/z 826.4 [M+H]⁺. ¹H NMR (400 MHz, DMSO-d₆) δ 11.83 (d, J=169.0 Hz, 2H), 7.89 (d, J=2.5 Hz, 1H), 7.37-7.10 (m, 9H), 6.92-6.74 (m, 4H), 4.04 (t, J=6.5 Hz, 2H), 3.72 (s, 10H), 3.45 (ddt, J=10.7, 6.8, 3.4 Hz, 2H), 2.98 (dddd, J=29.3, 20.2, 9.2, 5.5 Hz, 2H), 2.79 (p, J=6.8 Hz, 1H), 2.69 (q, J=6.1 Hz, 2H), 2.02-1.64 (m, 3H), 1.16-1.06 (m, 12H), 0.99 (t, J=6.9 Hz, 6H). ³¹P NMR (162 MHz, DMSO-d₆) δ 146.60 (d, J=12.0 Hz).

### Example 39—Preparation of Compound 313

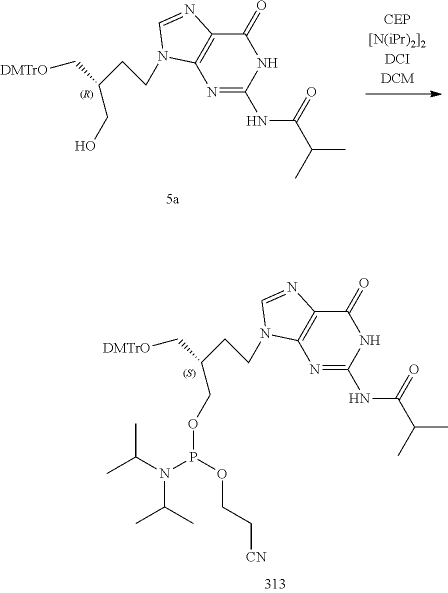

Preparation of 313: To a solution of 5a (1.8 g, 2.9 mmol) in DCM (18 mL) was added DCI (200 mg, 1.7 mmol) and CEP[N(Pr)₂]₂ (1.0 g, 3.5 mmol) under N₂. The mixture was stirred at r.t for 1 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated. The residue was purified by silica gel column chromatograph (eluent, PE/EA=1:1). The residue was purified by Flash-Prep-HPLC with the following conditions (IntelFlash-1): Column, C18 silica gel; mobile phase, CH₃CN/H₂O (0.05% NHAHCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 313 (1.0 g, 1.2 mmol, 41.3% yield, 99% purity) as a white solid. ESI-LCMS: m/z 826.4 [M+H]⁺. ¹H NMR (400 MHz, DMSO-d6) δ 12.07 (s, 1H), 11.64 (s, 1H), 7.89 (d, J=2.5 Hz, 1H), 7.38-7.09 (m, 9H), 6.94-6.75 (m, 4H), 4.05 (t, J=6.5 Hz, 2H), 3.86-3.39 (m, 12H), 2.99 (dddd, J=29.1, 20.0, 8.9, 5.2 Hz, 2H), 2.74 (dq, J=33.4, 6.5 Hz, 3H), 2.01-1.63 (m, 3H), 1.21-0.85 (m, 18H), ³¹P NMR (162 MHz, DMSO-d₆) δ 146.61 (d, J=11.9 Hz).

### Example 40—Preparation of R-Penciclovir & S-Penciclovir Intermediate 6R & 6S

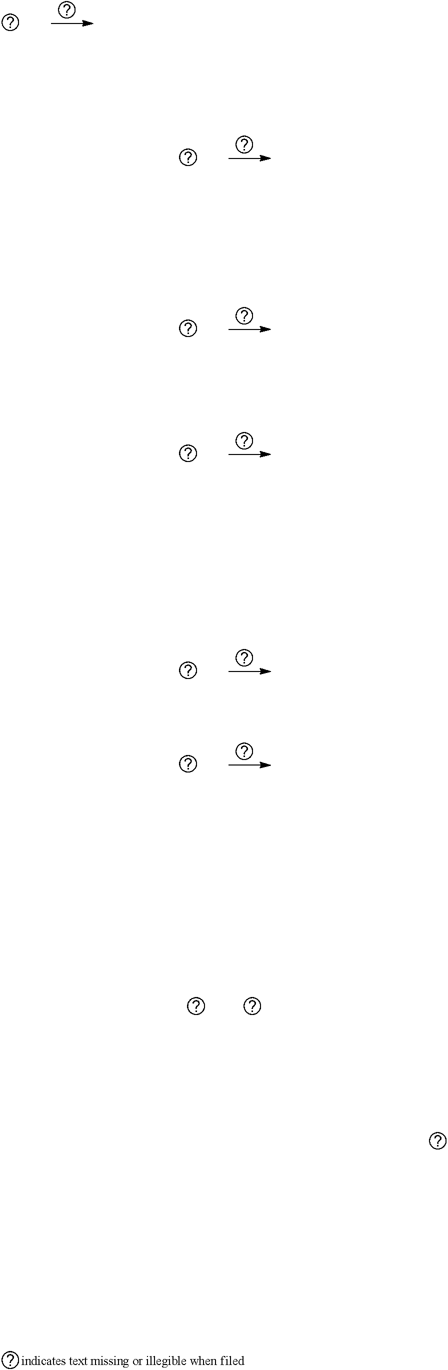

Preparation of 2: To a solution of 1 (30.0 g, 105.2 mmol) in DMF (300 mL) was added imidazole (30.0 g, 442.1 mmol) and TBSCl (39.7 g, 263.1 mmol) at r.t. The mixture was stirred at r.t for 15 h. LC-MS and TLC show SM was completely consumed. The mixture solution was added water, and the product was extracted with EA. And the combined organic phase was washed with water and saturated brine and dried over by Na₂SO₄. Then the solution was concentrated under reduced pressure. This resulted in crude 2 (65.0 g) as a white solid without further purified and used directly for the next step. ESI-LCMS: m/z 514.2 [M+H]+.

Preparation of 3: Crude 2 (65 g) was dried with pyridine for three times. To a solution of 2 (65.0 g) in Pyridine (600 mL) was added iBuCl (16.9 g, 157.9 mmol) at 0° C. The mixture was stirred at r.t. for 1 h. LC-MS showed 2 was consumed and confirmed the formation of the product. Water (500 mL) was added to the mixture to quench the reaction. The product was extracted into ethyl acetate (1.5 L). The organic layer was washed with brine and dried over anhydrous Na₂SO₄. The solution was then concentrated under reduced pressure to give crude 3 (86.0 g) as a white solid which was used directly for the next step. ESI-LCMS: m/z 584 [M+H]+.

Preparation of 4: To a solution of 3 (86.0 g) in THF (800 mL) was added TFA (400 mL) and water (400 mL) at 0° C. The reaction mixture was stirred at 0° C. for 2 h. LC-MS showed 3 was consumed completely. Con. NH₄OH was added to the mixture at 0° C. to quench the reaction until the pH=7.5. The product was extracted into ethyl acetate (2.5 L). The organic layer was washed with brine and dried over anhydrous Na₂SO₄. The solution was then concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=3/7 increasing to CH₃CN/H₂O (0.5% NH₄HCO₃)=1/0 within 20 min, the eluted product was collected at CH₃CN/H₂O (0.5% NH₄HCO₃)=7/3; Detector, UV 254 nm. This resulted in 4 (30.0 g, 63.9 mmol, 60.0% yield over 3 steps) as a white solid. ESI-LCMS: m/z 470.0 [M+H]+.

Preparation of 5: To a stirred solution of 4 (16 g, 21.0 mmol) and 2′OMe-S′vinyl-U amidite (10.9 g, 23.3 mmol) in ACN (160 mL) was added 0.25M ETT (88 ml, 23.3 mmol). The resulting mixture was stirred for 3 h at room temperature under argon atmosphere. LCMS showed 4 was consumed completely. The reaction mixture was used directly for next step.

Preparation of 6: The solution of 5 was added xanthane hydride (6.9 g, 46.6 mmol) and pyridine (7.3 g, 93.2 mmol) under nitrogen atmosphere. The resulting mixture was stirred for 0.5 h at room temperature under argon atmosphere. The reaction was quenched with water. The resulting mixture was extracted with EA (200 mL). The combined organic layers were washed with brine (1×200 mL), dried over anhydrous Na₂SO₄. After filtration, the filtrate was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in crude and purified by SFC to give 6R (4.2 g, 3.6 mmol) and 6S (4.5 g, 3.8 mmol) as a white solid.

### Example 41—Preparation of Compound 320

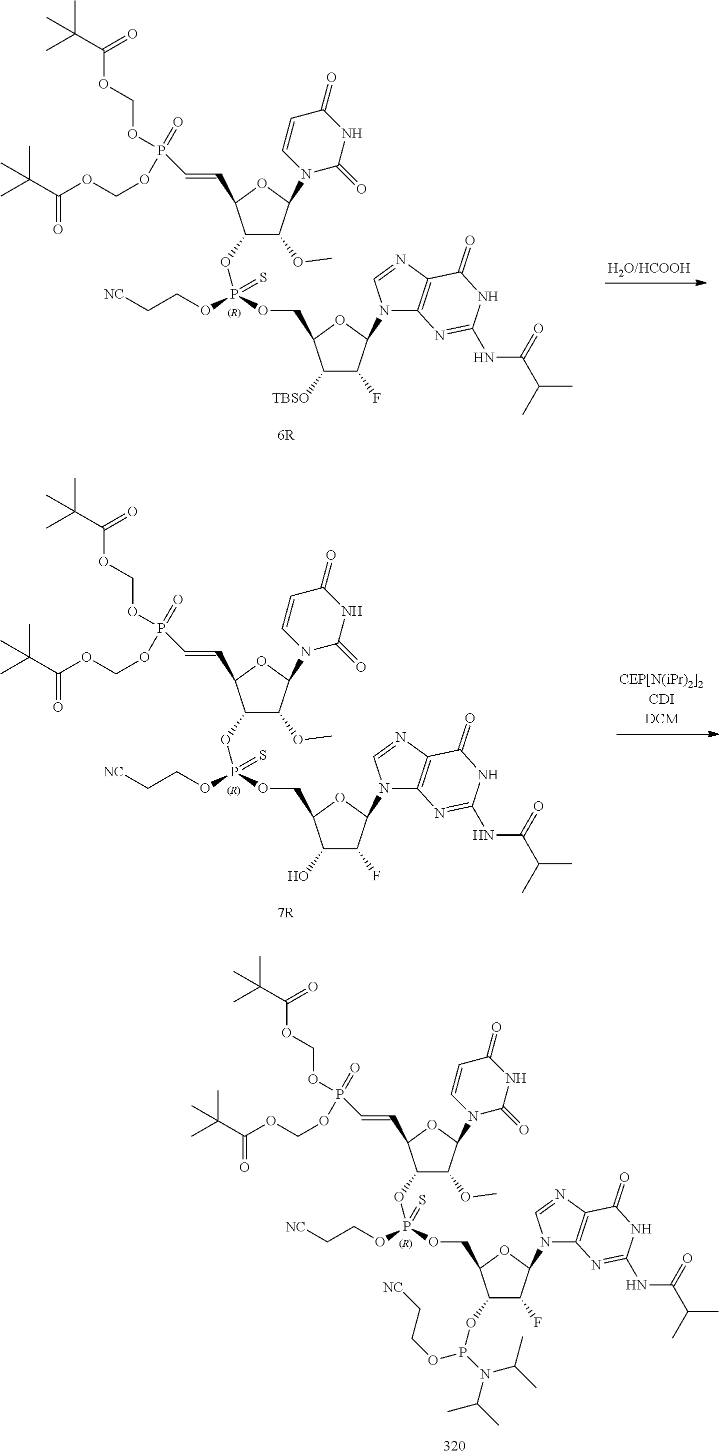

Preparation of compound 7R: To a solution of 6R (3.6 g, 3.1 mmol) in H₂O:HCOOH=1:1 (108 mL). Then the solution was stirred at r.t for 16 h. LCMS showed 6R was consumed completely. Then the solution was quench with NaHCO₃ (aq.) and diluted with EA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=3/1 within 40 min, the eluted product was collected at CH₃CN/H₂O (0.05% NH₄HCO₃)=3/2; Detector, UV 254 nm, filtered to get 7R (2.7 g, 2.6 mmol, 83.1% yield, 98% purity) as a solid, ESI-LCMS: m/z 1049.2 [M+H]⁺. ¹H NMR (400 MHz, DMSO-d₆) § 12.17 (s, 1H), 11.67 (s, 1H), 11.54 (d, J=2.2 Hz, 1H), 8.16 (s, 1H), 7.81 (d, J=8.1 Hz, 1H), 6.87 (ddd, J=22.9, 17.1, 5.5 Hz, 1H), 6.31-6.11 (m, 2H), 5.97 (d, J=6.5 Hz, 1H), 5.87 (d, J=6.0 Hz, 1H), 5.75 (dd, J=8.0, 1.9 Hz, 1H), 5.71-5.57 (m, 4H), 5.14 (dt, J=11.0, 4.7 Hz, 1H), 4.87-4.70 (m, 1H), 4.59 (dtd, J=21.1, 7.2, 4.7 Hz, 1H), 4.53-4.34 (m, 3H), 4.26 (dt, J=8.2, 5.7 Hz, 3H), 3.43 (s, 3H), 2.97 (t, J=5.9 Hz, 2H), 2.82 (hept, J=6.8 Hz, 1H), 1.23-1.16 (m, 24H).

Preparation of compound 320: To a solution of 7R (2.6 g, 2.5 mmol) in DCM (26 mL) was added DCI (249 mg, 2.1 mmol) and CEP[N(iPr)₂]₂ (1.1 g, 3.7 mmol) under N₂. The mixture was stirred at r.t for 1 h. LCMS showed 7R was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 320 (1.9 g, 1.5 mmol, 61.0% yield, 98% purity) as a white solid. ESI-LCMS: m/z 1249.4. ¹H NMR (400 MHz, DMSO-d₆) δ 12.13 (s, 1H), 11.72-11.31 (m, 2H), 8.14 (s, 1H), 7.77 (d, J=8.1 Hz, 1H), 6.84 (dddd, J=22.9, 17.0, 5.5, 2.1 Hz, 1H), 6.32-6.01 (m, 2H), 5.85 (d, J=6.1 Hz, 1H), 5.76-5.39 (m, 6H), 4.89-4.57 (m, 2H), 4.58-4.10 (m, 6H), 3.95-3.72 (m, 2H), 3.63 (dq, J=10.4, 6.7 Hz, 2H), 3.39 (d, J=2.2 Hz, 3H), 3.04-2.88 (m, 2H), 2.87-2.69 (m, 3H), 1.15 (dq, J=9.0, 4.8 Hz, 37H). ³¹P NMR (162 MHz, DMSO-d₆) δ150.49, 150.44, 150.39, 150.34, 67.35, 15.99.

### Example 42—Preparation of Compound 321

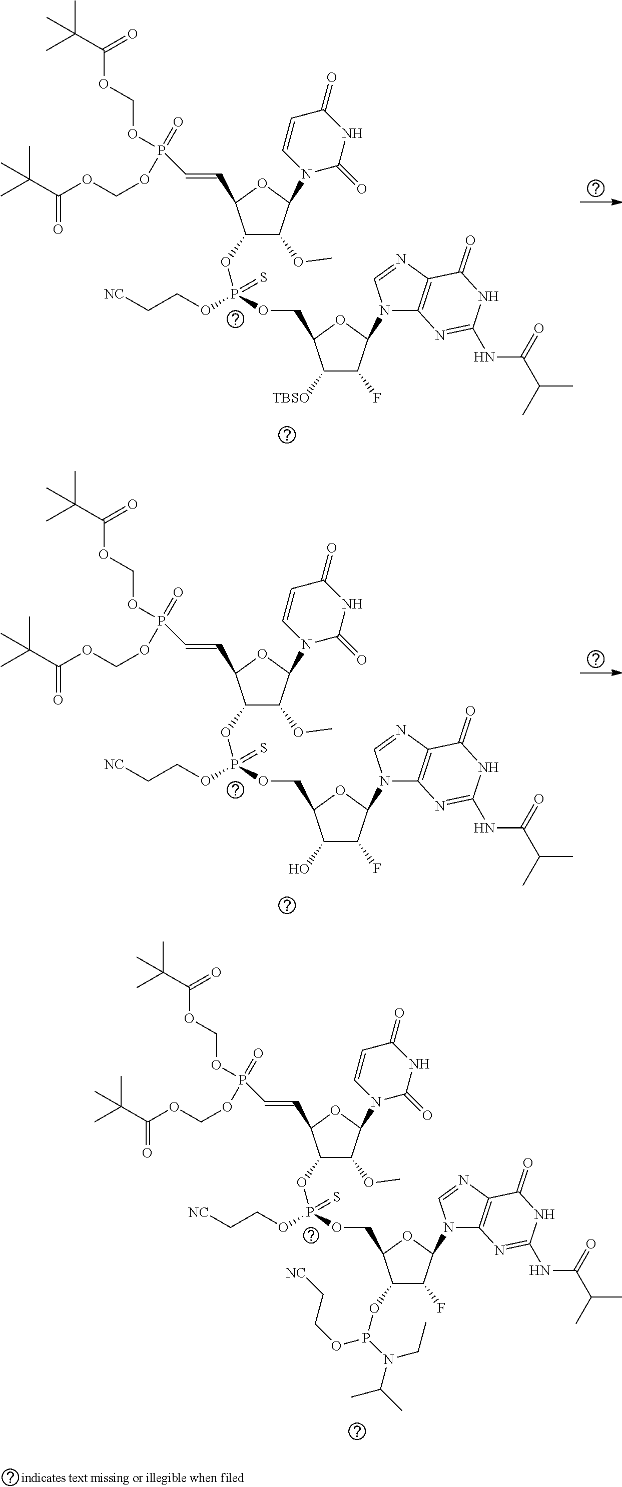

Preparation of compound 78: To a solution of 68 (3.8 g, 3.3 mmol) in H₂O:HCOOH=1:1 (114 mL). Then the solution was stirred at r.t for 16 h. LCMS showed 6S was consumed completely. Then the solution was quench with NaHCO₃ (aq.) and diluted with BA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NHAHCO₃)=3/1 within 40 min, the eluted product was collected at CH₃CN/H₂O (0.05% NH₄HCO₃)=3/2; Detector, UV 254 nm, filtered to get 78 (2.8 g, 2.7 mmol, 80.9% yield, 98% purity) as a solid, ESI-LCMS: m/z 1049.2 [M+H]⁺. ¹H NMR (400 MHz, DMSO-d₆) δ 12.17 (&, 1H), 11.67 (s, 1H), 11.54 (s, 1H), 8.16 (8, 1H), 7.81 (d, J=8.1 Hz, 1H), 6.88 (ddd, J=23.0, 17.1, 5.7 Hz, 1H), 6.32-6.12 (m, 2H), 5.97 (d, J=6.6 Hz, 1H), 5.86 (d, J=5.9 Hz, 1H), 5.74 (dd, J=8.1, 2.1 Hz, 1H), 5.71-5.59 (m, 4H), 5.15 (dt, J=11.0, 4.7 Hz, 1H), 4.76 (ddt, J=5.8, 4.1, 1.7 Hz, 1H), 4.65-4.43 (m, 3H), 4.41-4.17 (m, 4H), 2.99 (t, J=5.8 Hz, 2H), 2.81 (b, J=6.8 Hz, 1H), 1.23-1.15 (m, 24H).

Preparation of compound 321: To a solution of 78 (2.7 g, 2.6 mmol) in DCM (27 mL) was added DCI (258 mg, 2.2 mmol) and CEP[N(iPr)₂]₂ (1.2 g, 3.9 mmol) under N2. The mixture was stirred at r.t for 1 h. LCMS showed 7S was consumed completely. The product was extracted with DCM, The organic layer was washed with H₂O and brine. Then the solution was concentrated under reduced pressure and the residue was purified by Flash-Prep-HPLC with the following conditions (IntelFlash-1); Column, C18 silica gel, mobile phase, CH₃CN/H₂O (0.05% NHAHCO₃)=1/1 increasing to CH₃CN/H₂O (0.05% NHAHCO₃)=1/0 within 25 min, the eluted product was collected at CH₃CN/H₂O (0.05% NHAHCO₃)=1/0; Detector, UV 254 nm. This resulted in 321 (2.0 g, 1.6 mmol, 61.5% yield, 98% purity) as a white solid. ESI-LCMS: m/z 1249.4. ¹H NMR (400 MHz, DMSO-d₆) δ 12.13 (s, 1H), 11.77-11.33 (m, 2H), 8.14 (d, J=1.0 Hz, 1H), 7.78 (dd, J=8.2, 2.1 Hz, 1H), 6.29-6.08 (m, 2H), 5.84 (d, J=6.0 Hz, 1H), 5.75-5.40 (m, 6H), 5.19-5.05 (m, 1H), 4.83-4.58 (m, 2H), 4.57-4.30 (m, 4H), 4.24 (pd, J=5.4, 4.8, 2.7 Hz, 2H), 3.93-3.70 (m, 2H), 3.63 (dq, J=10.4, 6.8 Hz, 2H), 3.34 (d, J=2.1 Hz, 3H), 2.95 (q, J=5.7 Hz, 2H), 2.86-2.71 (m, 3H), 1.23-1.10 (m, 37H). ³¹P NMR (162 MHz, DMSO-d₆) δ 150.58, 150.55, 150.53, 150.50, 67.23, 67.21, 15.99.

### Example 43—Preparation of Compound 322

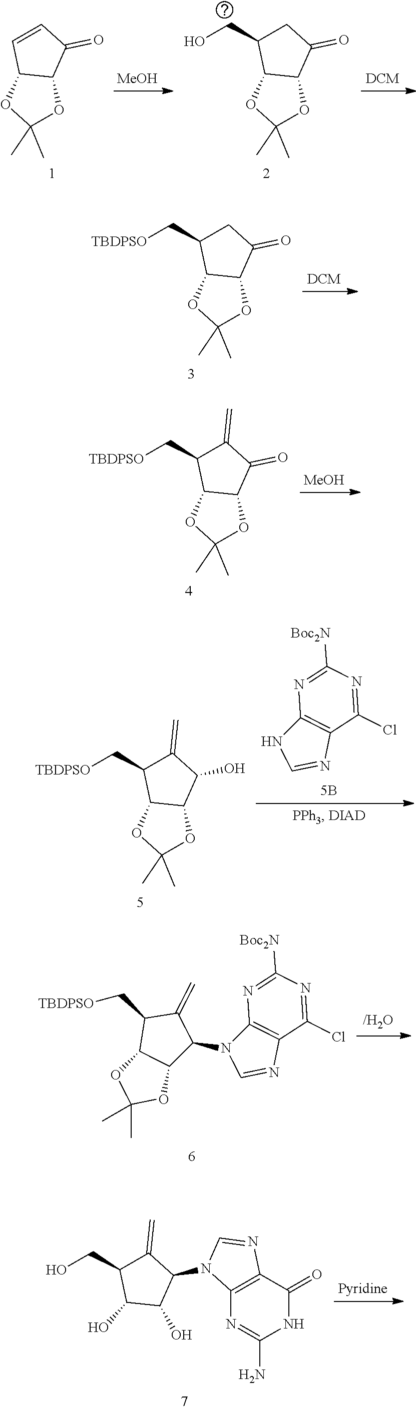

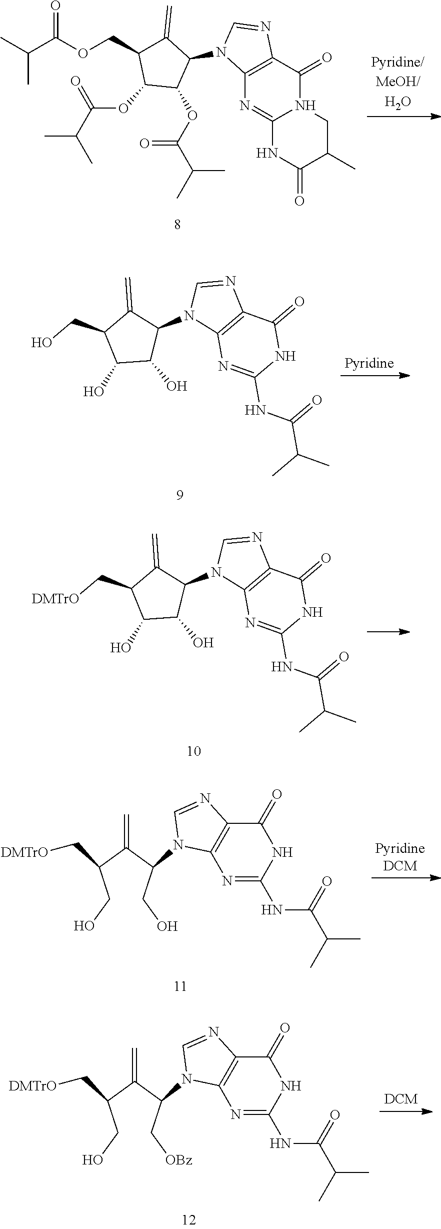

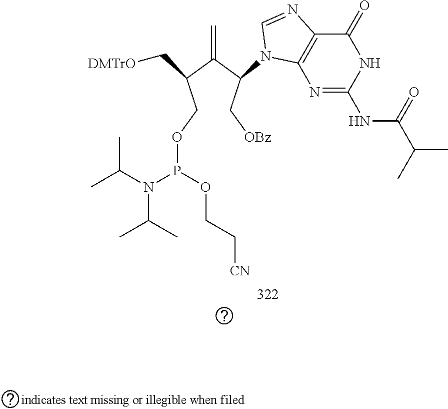

Preparation of 2: To a solution of compound 1 (50 g, 320 mmol) in MeOH (10 L) was added benzophenone (23.6 g, 130 mmol), the mixture was stirred at for 30 min under nitrogen atmosphere. Then the mixture was stirred for 45 min at room temperature under N₂ atmosphere and hv (λ=300 nm) until compound 1 was consumed, detected by TLC. The mixture was concentrated in vacuo and the resulting crude residue was purified by column chromatography (SiO₂, PE:EA=20:1-2:1) to give compound 2 (22.5 g, 37% yield) as a brown oil. ¹H NMR (400 MHz, DMSO-d₆) δ 4.93 (t, J=4.5 Hz, 1H), 4.64 (d, J=5.4 Hz, 1H), 4.18 (d, J=5.4 Hz, 1H), 3.62-3.54 (m, 1H), 3.47-3.39 (m, 1H), 2.59 (dd, J=18.0, 8.9 Hz, 1H), 2.43-2.34 (m, 1H), 1.32 (s, 3H), 1.27 (s, 3H).

Preparation of 3: To a solution of compound 2 (225 g, 1.2 mol) in anhydrous DCM (2300 mL) was added imidazole (246.8 g, 3.6 mol) and the mixture was stirred at room temperature for 30 min under nitrogen atmosphere, then TBDPSCl (298.9 g, 1.1 mol) was added to the mixture over 20 min. The mixture was stirred for 2 h until compound 2 was consumed, detected by TLC. The reaction was quenched with MeOH and extracted by EtOAc (2000 mL×3). The organic layer was washed with water (1000 mL×2), brine (1000 mL), dried over anhydrous sodium sulfate and evaporated in vacuo. The crude was purified by Flash-Prep-HPLC with the following conditions (IntelFlash-1): Column, C₁₈ silica gel; mobile phase, CH₃CN/H₂O (0.5% NH₄HCO₃)=3/1 increasing to CH₃CN/H₂O (0.5% NH₄HCO₃)=1/0 within 25 min, the eluted product was collected at CH₃CN/H₂O (0.5% NH₄HCO₃)=5/1; Detector, UV 254 nm. This resulted in compound 3 (325 g, 63% yield) as brown oil. ¹H NMR (400 MHz, CDCl₃) δ 7.64 (ddd, J=6.5, 3.2, 2.0 Hz, 4H), 7.50-7.40 (m, 6H), 4.67 (d, J=5.4 Hz, 1H), 4.39 (d, J=5.4 Hz, 1H), 3.83 (dd, J=10.2, 2.7 Hz, 1H), 3.63 (dd, J=10.2, 3.3 Hz, 1H), 2.78 (dd, J=18.4, 9.2 Hz, 1H), 2.52 (d, J=8.4 Hz, 1H), 2.31-2.15 (m, 1H), 1.46 (s, 3H), 1.37 (s, 3H), 1.05 (s, 9H).

Preparation of 4: To a solution of compound 3 (92 g, 216.7 mmol) in DCM (2000 mL) was added formaldehyde (32.5 g, 1.1 mol), propionic acid (1.6 g, 21.6 mmol) and tetrahydropyrrole (1.5 g, 21.6 mmol) at room temperature under nitrogen atmosphere. The mixture was stirred for 16 at r.t. under nitrogen atmosphere until compound 3 was consumed, detected by TLC and LCMS. The mixture was concentrated in vacuo to give crude product and the residue was purified by column chromatography (SiO₂, PE:EA=20:1) to give compound 4 (51 g, 50% yield) as yellow solid. ¹H NMR (400 MHz, CDCl₃) δ 7.66-7.62 (m, 2H), 7.58 (dd, J=8.0, 1.4 Hz, 2H), 7.46-7.38 (m, 6H), 6.34 (d, J=1.9 Hz, 1H), 5.50 (d, J=1.4 Hz, 1H), 4.69 (d, J=5.4 Hz, 1H), 4.60 (d, J=5.4 Hz, 1H), 3.92 (dd, J=10.0, 3.4 Hz, 1H), 3.79-3.74 (m, 1H), 3.09 (s, 1H), 1.40 (d, J=3.5 Hz, 6H), 1.03 (s, 9H); ESI-MS: m/z 454.3 [M+18]⁺.

Preparation of 5: To a solution of compound 4 (60 g, 137.4 mmol) in MeOH (900 mL) was added CeCl₃-7H₂O (44 g, 178.6 mmol) at room temperature, then the mixture was cooled to −78° C. and stirred for 15 min under nitrogen atmosphere. Then NaBH₄ (6.8 g, 178.6 mmol) was added slowly to the mixture over 10 min and the reaction was stirred at −78° C. for 15 min. Then the reaction was slowly warmed to 0° C. over 1 h and stirred at this temperature for 2 h until the compound 4 was consumed, detected by TLC and LCMS. The reaction was quenched by addition of saturated ammonium chloride solution and extracted by DCM (1200 mL×3). The combined organic layers was washed with water (1000 mL), brine (1000 mL), dried over anhydrous sodium sulfate and concentrated in vacuo to give resulting crude compound 5 as a yellow oil, which can be used directly for next step without further purification. ¹H NMR (400 MHz, DMSO-d₆) δ 7.64-7.57 (m, 4H), 7.48-7.41 (m, 6H), 5.10 (s, 1H), 4.97 (s, 1H), 4.77 (d, J=7.5 Hz, 1H), 4.50-4.39 (m, 3H), 3.60 (dd, J=5.7, 3.0 Hz, 2H), 1.30 (s, 3H), 1.25 (s, 3H), 0.99 (s, 9H); ESI-MS: m/z 461.2 [M+H]⁺.

Preparation of 6: To a solution of compound 5 (58 g, 132.2 mmol) in THF (600 mL) was added compound 5B (73.3 g, 198.3 mmol) and PPh₃ (51.9 g, 198.3 mmol), the mixture was cooled down to 0° C., then DIAD (40 g, 198.3 mmol) added dropwise. The mixture was stirred for 2 h at 50° C. under nitrogen atmosphere. LCMS and TLC (PE:EA=3:1) showed the reaction was completed. The reaction was quenched by addition of H₂O (10 mL) and extracted by EtOAc (400 mL×3). The combine organic layers were dried over anhydrous sodium sulfate and concentrated under reduced pressure to give the crude. The residue was purified by column chromatography (SiO₂, PE:EA=10:1-2:1) to give compound 6 (42 g, 40% yield for two steps) as a yellow oil. ¹H NMR (400 MHz, CDCl₃) δ 8.10 (s, 1H), 7.67-7.63 (m, 4H), 7.47-7.38 (m, 7H), 5.47 (m, 1H), 5.13 (m, 1H), 4.81 (t, J=6.0 Hz, 1H), 4.69-4.65 (m, 2H), 3.92 (m, 2H), 3.03 (m, 1H), 1.57 (s, 3H), 1.41 (s, 18H), 1.32 (s, 3H), 1.09 (s, 9H); ESI-MS: m/z 790.3 [M+H]⁺.

Preparation of 7: A solution of compound 6 (42 g, 53 mmol) in TFA (420 mL) and H₂O (42 mL) was stirred at r.t. overnight. LCMS showed the reaction was completed. The reaction mixture was concentrated under reduced pressure to give the crude, The crude was purified by Flash-Prep-HPLC with the following conditions (IntelFlash-1): Column, C₁₈ silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=1/5; Detector, UV 254 nm. This resulted in compound 7 (13.0 g, 70% yield) as white solid. ¹H NMR (400 MHz, DMSO-d₆) δ 7.59 (s, 1H), 6.69 (s, 2H), 5.03 (m, 3H), 4.47 (m, 1H), 4.35 (dd, 1H), 3.98 (m, 1H), 3.55 (m, 2H), 2.57 (m, 1H); ESI-MS: m/z 294.1 [M+H]⁺.

Preparation of 8: To a solution of compound 7 (11 g, 37.5 mmol) in dry pyridine (110 mL) was added drop wise isobutyryl chloride (20 g, 187.5 mmol) at 0° C., the mixture was stirred at r.t. for 2 h under nitrogen atmosphere. LCMS showed the reaction was completed. The reaction was quenched by addition of H₂O (10 mL) and extracted by EtOAc (200 mL×3). The combine organic layers were dried over anhydrous sodium sulfate and concentrated under reduced pressure to give a crude without further purification. ESI-MS: m/z 574.3 [M+H]⁺.

Preparation of 9: To a solution of compound 8 (15 g, 26.4 mmol) in pyridine (300 mL) was dropwised 2 N NaOH (in MeOH:H₂O=4:1, 240 mL) at 0° C., the mixture was stirred for 1 h at 0° C., LCMS showed the reaction was completed, The PH of mixture was adjusted to 7 by 4N HCl at 0° C. The reaction mixture was concentrated under reduced pressure to give the crude, The crude was purified by Flash-Prep-HPLC with the following conditions (IntelFlash-1): Column, C₁₈ silica gel; mobile phase, CH₃CN/H₂O (0.5% NH₄HCO₃)=0/1 increasing to CH₃CN/H₂O (0.5% NH₄HCO₃)=1/3 within 25 min, the eluted product was collected at CH₃CN/H₂O (0.5% NH₄HCO₃)=1/5; Detector, UV 254 nm. This resulted in compound 9 (9.0 g, 65% yield for two steps) as white solid. ESI-MS: m/z 364.1 [M+H]⁺.

Preparation of 10: To a solution of compound 9 (9.0 g, 24.7 mmol) in pyridine (90 mL) was added DMTr-Cl (10 g, 29.7 mmol), the mixture was stirred for 16 h at r.t under nitrogen atmosphere. LCMS showed the reaction was completed, the reaction was added NaHCO₃ solution, then extracted with EA, dried over anhydrous Na₂SO₄ and filtered. The filtrate was concentrated under reduced pressure to give a residue. The crude was filtered by column chromatography (SiO₂, 10% MeOH in DCM) to give compound 10 (10.8 g, 65% yield) as a white solid. ¹H NMR (400 MHz, DMSO-d₆) δ 12.03 (s, 1H), 11.67 (s, 1H), 7.78 (s, 1H), 7.42-7.17 (m, 12H), 7.07 (m, 2H), 6.90 (m, 4H), 6.84 (m, 2H), 5.37 (d, J=5.5 Hz, 1H), 5.22 (m, 1H), 4.99 (d, J=3.1 Hz, 1H), 4.83 (m, 1H), 4.46 (m, 1H), 4.34 (m, 1H), 4.01 (m, 1H), 3.74-3.72 (m, 10H), 3.34 (m, 2H), 2.74 (m, 1H), 2.63 (m, 1H), 1.11 (m, 6H). ESI-MS: m/z 666.3 [M+H]⁺.

Preparation of 11: NaIO₄ (5.14 g, 24 mmol) was dissolved in 10 mL of hot water, to the hot solution was added silica gel (20 g) with vigorous swirling and sharking. To a solution of 10 (8.7 g, 13.1 mmol) in DCM (170 mL) was added NaIO₄ silica gel (26 g). The resulting mixture was stirred for 30 min at room temp, TLC showed 10 was consumed completely. Then filtered and the filtrate was added NaBH₄ (546 mg, 14.3 mmol). The resulting mixture was stirred for 30 min at room temp, the reaction was added NH₄Cl solution, then extracted with DCM, dried over anhydrous Na₂SO₄ and filtered. The filtrate was concentrated under reduced pressure to give a residue. The crude was purified by Flash-Prep-HPLC with the following conditions (IntelFlash-1): Column, C18 silica gel; mobile phase, CH₃CN/H₂O (0.5% NH₄HCO₃)=0/1 increasing to CH₃CN/H₂O (0.5% NH₄HCO₃)=1/1 within 25 min, the eluted product was collected at CH₃CN/H₂O (0.5% NH₄HCO₃)=1/3; Detector, UV 254 nm. This resulted in the compound 11 (3.9 g, 44% yield) as a white solid. ¹H NMR (400 MHz, DMSO-d₆) δ 12.07 (s, 1H), 11.56 (s, 1H), 8.00 (s, 1H), 7.35-7.14 (m, 10H), 6.85 (m, 4H), 5.34 (s, 1H), 5.04 (t, J=5.2 Hz, 1H), 4.99 (s, 1H), 4.88 (m, 1H), 4.55 (t, J=7.6 Hz, 1H), 4.12 (m, 1H), 3.87 (m, 1H), 3.73 (s, 1H), 3.72 (s, 6H), 3.09 (m, 1H), 2.93 (m, 1H), 2.79 (m, 1H), 2.41 (m, 1H), 1.11 (m, 6H); ESI-MS: m/z 668.3 [M+H]⁺.

Preparation of 12: To a solution of 11 (3.9 g, 5.8 mmol) in DCM (40 mL) was added pyridine (5.2 g, 58.4 mmol), BzCl (1.0 g, 7.6 mmol) was slowly added at −78° C. The resulting mixture was stirred for 2 h at −78° C. TLC showed 11 was consumed completely. The reaction was added NaHCO₃ solution, then extracted with DCM, dried over anhydrous Na₂SO₄ and filtered. The filtrate was concentrated under reduced pressure to give a residue. The crude was purified by Flash-Prep-HPLC with the following conditions (IntelFlash-1): Column, C₁₈ silica gel; mobile phase, CH₃CN/H₂O (0.5% NH₄HCO₃)=1/5 increasing to CH₃CN/H₂O (0.5% NH₄HCO₃)=3/1 within 25 min, the eluted product was collected at CH₃CN/H₂O (0.5% NH₄HCO₃)=1/1; Detector, UV 254 nm and column chromatography (SiO₂, 10% MeOH in DCM). This resulted in the compound 12 (1.5 g, 33% yield) and 3′OBz (900 mg) as a white solid. ¹H NMR (400 MHz, DMSO-d₆) δ 12.12 (s, 1H), 11.62 (s, 1H), 8.17 (s, 1H), 7.78 (m, 2H), 7.66 (m, 1H), 7.50 (m, 2H), 7.38-7.11 (m, 9H), 6.88 (m, 4H), 5.64 (s, 1H), 5.38 (m, 1H), 5.26 (s, 1H), 4.98 (m, 2H), 4.73 (m, 1H), 3.78 (s, 6H), 3.50 (m, 2H), 3.32 (m, 2H), 2.81 (m, 1H), 2.48 (m, 1H), 1.11 (m, 6H); ESI-MS: m/z 772.3 [M+H]⁺.

Preparation of 322: To a solution of 12 (1.5 g, 1.9 mmol) in DCM (25 mL) was added DCI (137 mg, 1.1 mmol), CEP[N(iPr)₂]₂ (760 mg, 2.5 mmol). The resulting mixture was stirred for 1 h at room temperature, TLC showed 12 was consumed completely. The reaction was added NaHCO₃ solution, then extracted with DCM, dried over anhydrous Na₂SO₄ and filtered. The filtrate was concentrated under reduced pressure to give a residue. The crude was filtered by Flash-Prep-HPLC with the following conditions (IntelFlash-1): Column, C₁₈ silica gel; mobile phase, CH₃CN/H₂O (0.5% NH₄HCO₃)=1/3 increasing to CH₃CN/H₂O (0.5% NH₄HCO₃)=4/1 within 25 min, the eluted product was collected at CH₃CN/H₂O) (0.5% NH₄HCO₃)=7/3; Detector, UV 254 nm. This resulted in the compound 322 (1.3 g, 68% yield) as a white solid. ¹H NMR (400 MHz, DMSO-d₆) δ 12.04 (s, 1H), 11.48 (s, 1H), 8.17 (m, 1H), 7.72 (m, 2H), 7.61 (m, 1H), 7.44 (m, 2H), 7.29-7.06 (m, 9H), 6.80 (m, 4H), 5.64 (s, 1H), 5.28 (m, 2H), 4.97 (m, 1H), 4.86 (m, 1H), 3.70 (s, 6H), 3.64-3.35 (m, 6H), 3.19 (m, 1H), 3.08 (m, 1H), 2.75-2.58 (m, 4H), 1.09 (m, 12H), 0.95 (m, 6H); ³¹P NMR (162 MHz, DMSO-d₆) δ 146.45, 146.44; ESI-MS: m/z 973.3 [M+H]⁺.

### Example 44—Preparation of Compound 323

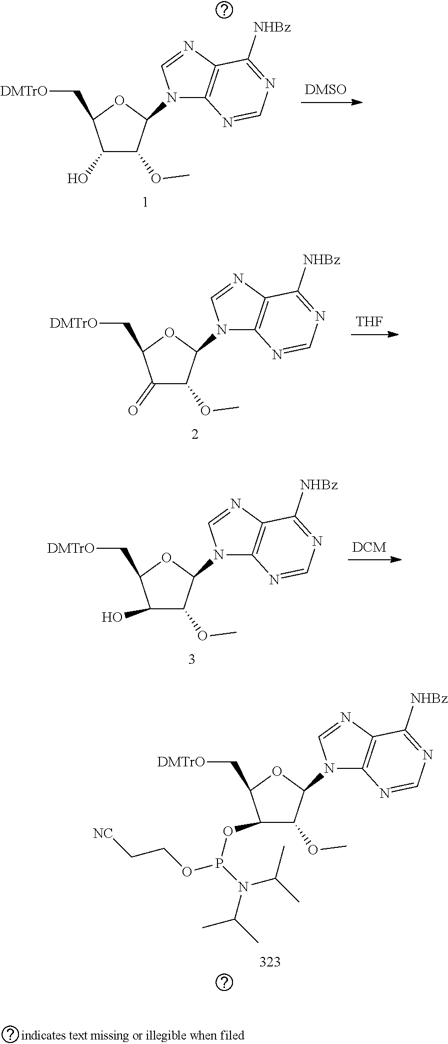

Preparation of compound 2: To a solution of 1 (15.0 g, 21.8 mmol) in DMSO (150 mL) was added EDCI (12.5 g, 65.4 mmol), and the mixture was cooled to 0° C. The mixture solution was added pyridine (1.9 g, 23.9 mmol) and TFA (1.2 g, 11 mmol) at 0° C. The mixture solution was stirred at 25° C. for 4 h. TLC and LCMS show 1 was completely consumed. Then the solution was added water (2 L), the product was extracted with EA, and was washed with water. Then washed the organic phase once with saturated brine and dried over by Na₂SO₄. Then the solution was concentrated under reduced pressure. The residue was to give 2 (15.5 g) as a white solid. ESI-LCMS: m/z 686 [M+H]⁻.

Preparation of compound 3: To a solution of 2 (15.5 g) in THE (160 mL) was added TBAB (3.9 g, 45 mmol) at 0° C. The mixture solution was stirred at 0° C. for 1 b. LC-MS show SM was completely consumed. Then the solution was added NH₄Cl·aq (200 mL), the product was extracted with BA, and was washed with water. Then washed the organic phase once with saturated brine and dried over by Na₂SO₄. Then the solution was concentrated under reduced pressure. The residue was purified by Flash-Prep-HPLC with the following conditions (IntelFlash-1): Column, C18 silica gel; mobile phase, CH₃CN/H₂O (0.5% NHAHCO₃)=2/3 increasing to CH₃CN/H₂O (0.5% NH₄HCO₃)=3/1 within 40 min, the eluted product was collected at CH₃CN/H₂O (0.5% NHAHCO₃)=3/2; Detector, UV 254 nm, this resulted in 3 (5.1 g, 7.3 mmol, 34% yield over 2 steps) as a white solid. ESI-LCMS: m/z 688 [M+H]⁻. ¹H NMR (400 MHz, DMSO-d₆) δ 11.22 (s, 1H), 8.77 (s, 1H), 8.31 (s, 1H), 8.10-8.00 (m, 2H), 7.70-7.14 (m, 12H), 6.91-6.78 (m, 4H), 6.29-6.20 (m, 1H), 5.73-5.61 (m, 1H), 4.43-4.32 (m, 1H), 4.27-4.19 (m, 1H), 4.13 (s, 1H), 3.75-3.68 (m, 6H), 3.51-3.41 (m, 3H), 3.31-3.25 (m, 1H).

Preparation of compound 323: To a solution of 3 (5.0 g, 7.3 mmol) in DCM (50 mL) was added DCI (729 mg, 6.1 mmol) and CEP[N(Pr)₂]₂ (2.6 g, 8.7 mmol) at r.t at N₂. The mixture was stirred at r.t. for 2 h under Na atmosphere. LCMS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated to give the crude. The crude was purified by Flash-Prep-HPLC with the following conditions (IntelFlash-1). Column, C18 silica gel; mobile phase, CH₃CN/H₂O=3/2 increasing to CH₃CN/H₂O=1/0 within 20 min, the eluted product was collected at CH₃CN/H₂O J=1/0. This resulted in 323 (5.1 g, 5.7 mmol, 80% yield) as a white solid. ESI-LCMS: m/z 888 [M+H]⁻. ¹H NMR (400 MHz, DMSO-d₆) δ 11.21 (s, 1H), 8.81-8.72 (m, 2H), 8.21-8.13 (m, 1H), 8.08-7.98 (m, 2H), 7.69-7.38 (m, 5H), 7.35-7.08 (m, 7H), 6.92-6.79 (m, 4H), 6.31 (s, 1H), 4.62-4.52 (m, 1H), 4.49-4.34 (m, 2H), 3.77-3.66 (m, 6H), 3.64-3.41 (m, 6H), 3.37-3.32 (m, 1H), 3.26-3.12 (m, 2H), 2.68-2.51 (m, 2H), 1.06-0.94 (m, 6H), 0.86-0.69 (m, 6H). ³¹P NMR (400 MHz, DMSO-d₆) δ 150.14, 147.16.

### Example 45—Preparation of Compound 324

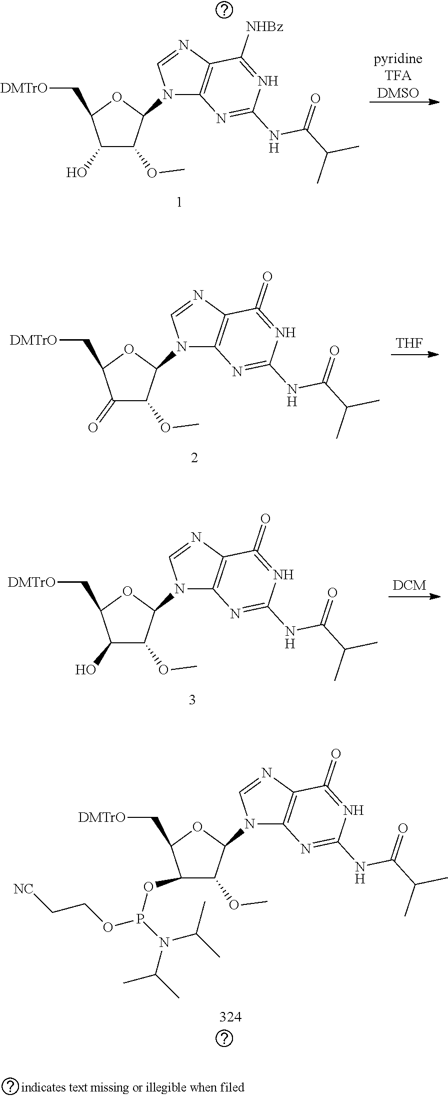

Preparation of compound 2: To a solution of 1 (20.0 g, 29.9 mmol) in DMSO (200 mL) added EDCI (17.2 g, 89.7 mmol) under N₂ atmosphere. Then the mixture was added pyridine (2.60 g, 32.9 mmol) and TEA (1.70 g, 14.9 mmol) in 0° C. The mixture was stirred at r.t. for 1 h. Then the solution was diluted with EA, washed with water twice and brine. The organic phase dried over anhydrous Na₂SO₄, filtered and concentrated under reduced pressure to get a crude 2 (16.5 g crude). The crude was used to next step directly. ESI-LCMS: m/z 668 [M+H]⁺.

Preparation of compound 3: To a solution of 2 (16.5 g, 24.7 mmol) in THE (160 mL). Then the solution was added borane tert-butylamine complex (4.3 g, 49.4 mmol) in 0° C. The mixture was stirred at 0° C. for 1 h. Then the solution was quench with NH₄Cl (aq.) and diluted with EA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/1 within 30 min, the eluted product was collected at CH₃CN/H₂O (0.5% NH₄HCO₃)=3/2; Detector, UV 254 nm, filtered to get 3 (6.5 g, 9.7 mmol, 39.2% yield, 99% purity) as a solid. ESI-LCMS: m/z 670.3 [M+H]⁺. ¹H NMR (400 MHz, DMSO-d₆) δ 11.88 (s, 2H), 7.90 (s, 1H), 7.47-7.35 (m, 2H), 7.35-7.14 (m, 7H), 6.91-6.80 (m, 4H), 5.94 (d, J=1.4 Hz, 1H), 5.65 (s, 1H), 4.31 (dt, J=7.6, 3.5 Hz, 1H), 4.07-3.98 (m, 1H), 3.73 (d, J=1.6 Hz, 6H), 3.43 (dd, J=10.3, 7.9 Hz, 1H), 3.42 (s, 3H), 3.26 (dd, J=10.3, 3.3 Hz, 1H), 2.80 (p, J=6.8 Hz, 1H), 1.27-1.01 (m, 6H).

Preparation of compound 324: To a solution of 3 (6.5 g, 9.7 mmol) in DCM (65 mL) was added DCI (973.0 mg, 8.24 mmol) and CEP[N(iPr)₂]₂ (3.51 g, 11.64 mmol) under N₂. The mixture was stirred at 35° C. for 2 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=5/6; Detector, UV 254 nm. This resulted in 324 (5.6 g, 6.4 mmol, 65.9% yield) as a white solid. ESI-LCMS: m/z 870.2 [M+H]+; ¹H NMR (400 MHz, DMSO-d₆) δ 12.15 (s, 1H), 11.67 (s, 1H), 7.77 (ddd, J=5.4, 3.3, 1.8 Hz, 1H), 7.42 (ddd, J=8.7, 3.8, 1.9 Hz, 2H), 7.34-7.17 (m, 7H), 6.86 (td, J=9.0, 5.8 Hz, 4H), 6.01 (tt, J=3.2, 1.3 Hz, 1H), 5.78-5.72 (m, 1H), 4.54-4.38 (m, 2H), 4.22 (d, J=2.8 Hz, 1H), 3.73 (dd, J=3.0, 0.9 Hz, 6H), 3.64 (ddt, J=10.5, 7.7, 5.3 Hz, 1H), 3.55-3.41 (m, 5H), 3.40-3.13 (m, 3H), 2.87-2.75 (m, 1H), 2.72-2.55 (m, 2H), 1.13 (d, J=6.7 Hz, 6H), 1.04 (t, J=6.5 Hz, 6H), 0.85 (dd, J=6.8, 2.3 Hz, 6H). ³¹P NMR (162 MHz, DMSO-d₆) δ 150.63, 146.47.

### Example 46—Preparation of Compound 341

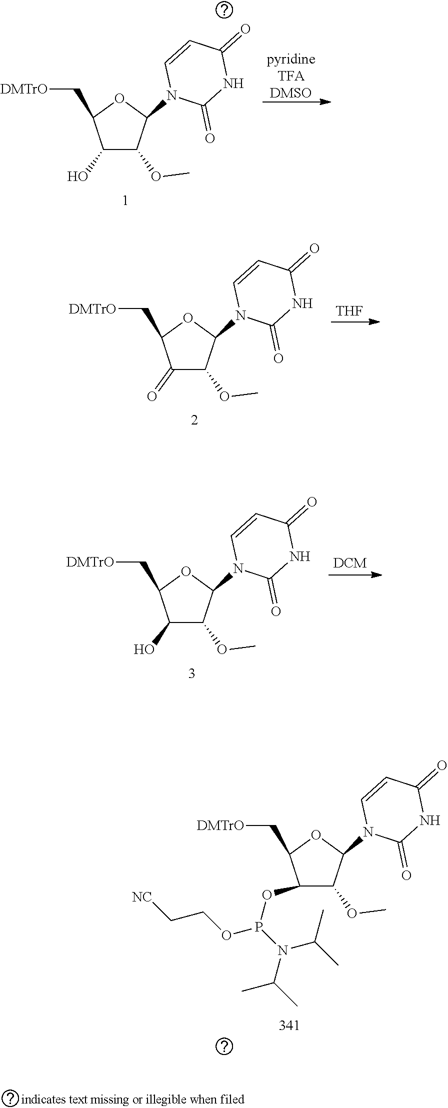

Preparation of compound 2: To a solution of 1 (20.0 g, 35.7 mmol) in DMSO (200 mL) added EDCI (20.5 g, 107.1 mmol) under N₂ atmosphere. Then the mixture was added pyridine (3.1 g, 39.3 mmol) and TEA (2.0 g, 17.8 mmol) in 0° C. The mixture was stirred at r.t. for 1 h. Then the solution was diluted with EA, washed with water twice and brine. The organic phase dried over anhydrous Na₂SO₄, filtered and concentrated under reduced pressure to get a crude 2 (21 g crude). The crude was used to next step directly. ESI-LCMS: m/z 559 [M+H].

Preparation of compound 3: To a solution of 2 (21 g crude, 35.7 mmol) in THE (200 ml). Then the solution was added borane tert-butylamine complex (3.1 g, 35.7 mmol) in 0° C. The mixture was stirred at 0° C. for 1 h. Then the solution was quench with NH₄Cl (aq.) and diluted with EA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/1 within 40 min, the eluted product was collected at CH₃CN/H₂O (0.5% NH₄HCO₃)=3/2; Detector, UV 254 nm, filtered to get 3 (4.8 g, 8.6 mmol, 24.0% yield, 99% purity) as a solid, ESI-LCMS: m/z 559.3 [M+H]⁺. ¹H NMR (400 MHz, DMSO-d₆) δ 11.36 (s, 1H), 7.52 (d, J=8.2 Hz, 1H), 7.46-7.39 (m, 2H), 7.35-7.18 (m, 7H), 6.93-6.84 (m, 4H), 5.82 (d, J=1.1 Hz, 1H), 5.58-5.50 (m, 2H), 4.21 (dt, J=7.3, 3.3 Hz, 1H), 4.08-4.03 (m, 2H), 3.74 (s, 6H), 3.39 (s, 3H), 3.44-3.30 (m, 1H), 3.25 (dd, J=10.5, 3.5 Hz, 1H).

Preparation of compound 341: To a solution of 3 (4.8 g, 8.6 mmol) in DCM (50 mL) was added DCI (863.3 mg, 7.3 mmol) and CEP[N(iPr)₂]₂ (3.1 g, 10.3 mmol) under N2. The mixture was stirred at 35° C. for 2 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NHAHCO₃)=5/6; Detector, UV 254 nm. This resulted in 341 (5.0 g, 6.6 mmol, 76.4% yield) as a white solid. ESI-LCMS: m/z 859.3 [M+H]+; ¹H NMR (400 MHz, DMSO-d₆) δ 11.40 (s, 1H), 7.52-7.14 (m, 9H), 6.94-6.82 (m, 4H), 5.83 (dd, J=3.0, 0.9 Hz, TH), 5.46 (dd, J=25.9, 8.1 Hz, 1H), 4.45-4.23 (m, 2H), 3.90 (d, J=44.5 Hz, 1H), 3.80-3.46 (m, 8H), 3.46-3.16 (m, 7H), 2.74-2.65 (m, 1H), 2.55 (td, J=6.9, 6.5, 5.0 Hz, 1H), 1.05 (t, J=7.2 Hz, 6H), 0.86 (dd, J=23.1, 6.7 Hz, 6H), ³¹P NMR (162 MHz, DMSO-d₆) δ 150.43, 147.02.

### Example 47—Preparation of Compound 342

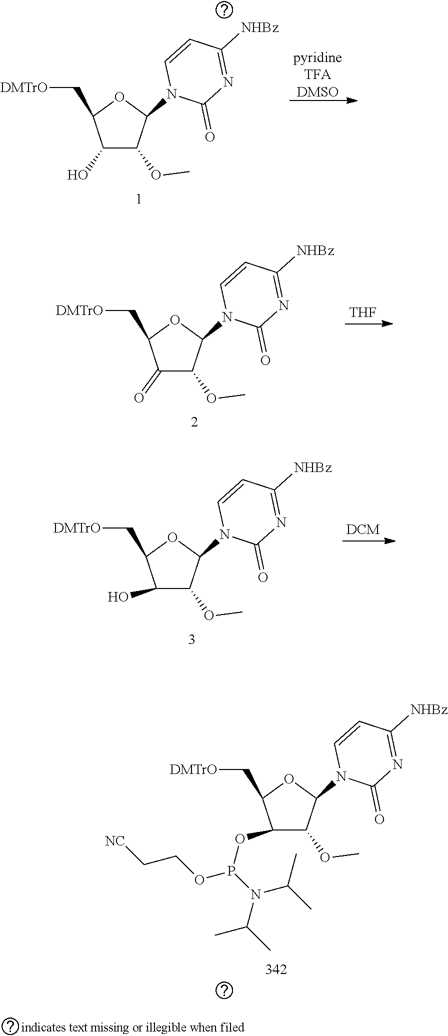

Preparation of compound 2: To a solution of 1 (20 g, 30.1 mmol) in DMSO (200 mL) added EDCI (17.3 g, 90.4 mmol) under Na atmosphere. Then the mixture was added pyridine (2.6 g, 33.1 mmol) and TFA (1.7 g, 15.1 mmol) in 0° C. The mixture was stirred at r.t. for 1 h. Then the solution was diluted with EA, washed with water twice and brine. The organic phase dried over anhydrous Na₂SO₄, filtered and concentrated under reduced pressure to get a crude 2 (19 g). The crude was used to next step directly. ESI-LCMS: m/z 662 [M+H]+.

Preparation of compound 3: To a solution of 2 (18 g, 27.2 mmol) in THF (180 mL). Then the solution was added NaBH₄ (514.1 mg, 13.6 mmol) in-78° C. The mixture was stirred at −78° C. for 1 h. Then the solution was quench with NH₄Cl (aq.) and diluted with EA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.5% NHHCO₃)=2/3 increasing to CH₃CN/H₂O (0.5% NH₄HCO₃)=3/1 within 40 min, the eluted product was collected at CH₃CN/H₂O (0.5% NH₄HCO₃)=3/2; Detector, UV 254 nm, filtered to get 3 (7.3 g, 10.8 mmol, 40.0% yield, 99.0% purity) as a solid, ESI-LCMS: m/z 664 [M+H]⁺, ¹H NMR (400 MHz, DMSO-d₆) δ 11.26 (s, 1H), 8.05-7.97 (m, 2H), 7.88 (d, J=7.5 Hz, 1H), 7.63 (t, J=7.4 Hz, 1H), 7.56-7.43 (m, 4H), 7.38-7.20 (m, 8H), 6.96-6.88 (m, 4H), 5.84 (s, 1H), 5.35 (d, J=3.3 Hz, 1H), 4.36 (dt, J=7.1, 3.1 Hz, 1H), 4.06 (t, J=3.3 Hz, 1H), 3.76 (s, 7H), 3.48 (s, 3H), 3.38-3.30 (m, 1H).

Preparation of compound 342: To a solution of 3 (6.5 g, 9.7 mmol) in DCM (65 mL) was added DCI (982 mg, 8.3 mmol) and CEP[N(Pr)₂]₂ (3.5 g, 11.7 mmol) under N₂. The mixture was stirred at 35° C. for 2 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NHAHCO₃)=5/6; Detector, UV 254 am. This resulted in 342 (5.7 g, 6.5 mmol, 66.7% yield) as a white solid. ESI-LCMS: m/z 864.1 [M+H]⁺; ¹H NMR (600 MHz, DMSO-d₆) δ 11.29 (s, 1H), 8.45 (dd, J=41.3, 7.5 Hz, 1H), 8.03-7.98 (m, 2H), 7.63 (t, J=7.4 Hz, 1H), 7.55-7.49 (m, 2H), 7.47-7.24 (m, 10H), 7.16-7.10 (m, 1H), 6.92 (dd, J=10.9, 8.6 Hz, 4H), 5.97-5.90 (m, 1H), 4.50 (did, J=79.6, 8.8, 4.7 Hz, 1H), 4.19 (dq, J=8.3, 5.0, 3.8 Hz, 1H), 4.01-3.93 (m, 1H), 3.65 (dd, J=132.2, 4.6 Hz, 10H), 3.38 (td, J=11.0, 3.5 Hz, 1H), 2.77 (1, J=5.9 Hz, 1H), 2.65-2.55 (m, 1H), 1.12 (dd, J=26.7, 6.7 Hz, 9H), 0.96 (d, J=6.7 Hz, 3H). ³¹P NMR (243 MHz, DMSO-d₆) δ 149.54, 148.83.

### Example 48—Preparation of Compound 344

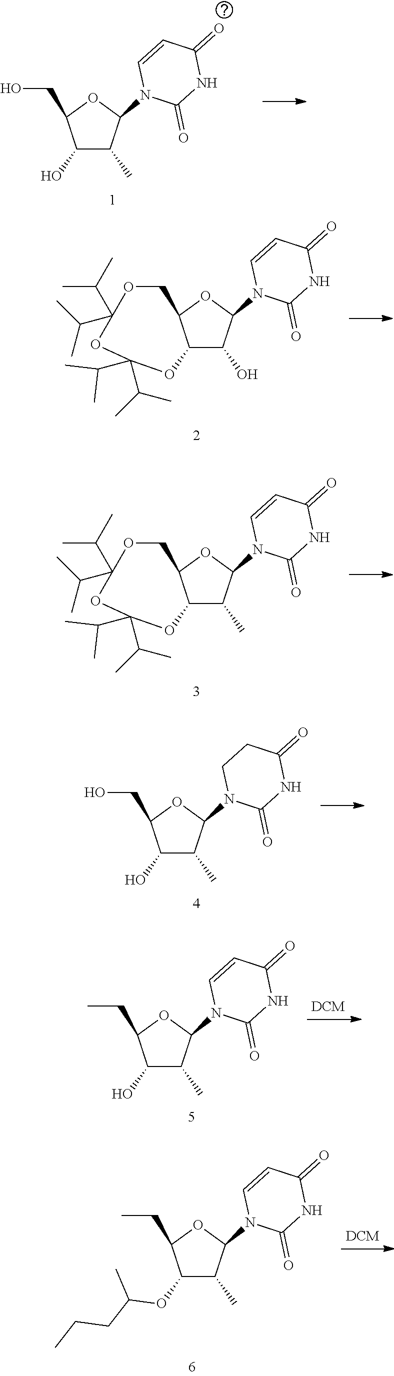 [SMILES: C[C@@H]1[C@H](O)[C@@H](CO)O[C@H]1n1ccc(=O)[nH]c1=O ; CC(C)C1(C(C)C)OC[C@H]2O[C@@H](n3ccc(=O)[nH]c3=O)[C@H](O)[C@@H]2OC(C(C)C)(C(C)C)O1 ; CC(C)C1(C(C)C)OC[C@H]2O[C@@H](n3ccc(=O)[nH]c3=O)[C@H](C)[C@@H]2OC(C(C)C)(C(C)C)O1 ; C[C@@H]1[C@H](O)[C@@H](CO)O[C@H]1N1CCC(=O)NC1=O ; CC[C@H]1O[C@@H](n2ccc(=O)[nH]c2=O)[C@H](C)[C@@H]1O ; CCCC(C)O[C@H]1[C@@H](C)[C@H](n2ccc(=O)[nH]c2=O)O[C@@H]1CC]

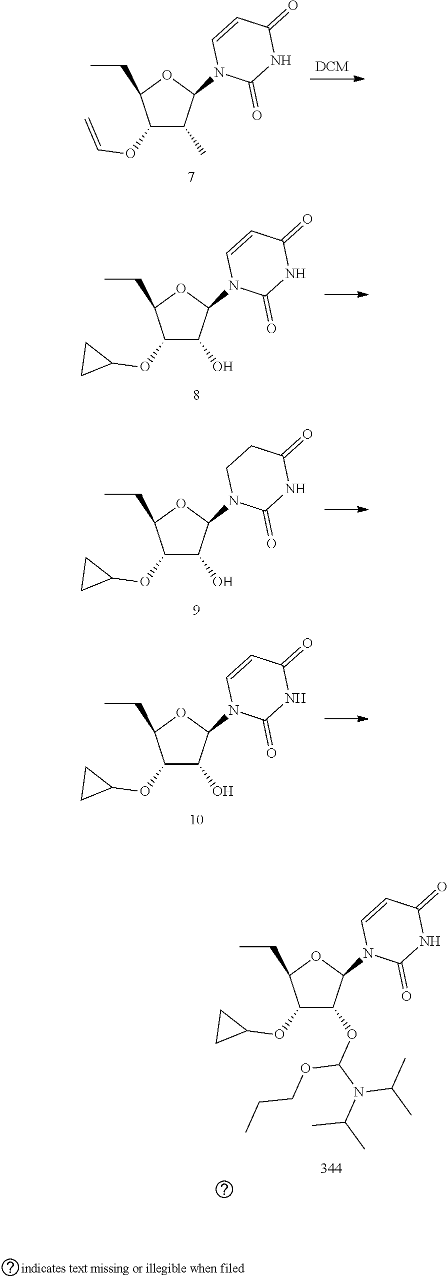 [SMILES: C=CO[C@H]1[C@@H](C)[C@H](n2ccc(=O)[nH]c2=O)O[C@@H]1CC ; CC[C@H]1O[C@@H](n2ccc(=O)[nH]c2=O)[C@H](O)[C@@H]1OC1CC1 ; CC[C@H]1O[C@@H](N2CCC(=O)NC2=O)[C@H](O)[C@@H]1OC1CC1 ; CC[C@H]1O[C@@H](n2ccc(=O)[nH]c2=O)[C@H](O)[C@@H]1OC1CC1 ; CCCOC(O[C@@H]1[C@H](OC2CC2)[C@@H](CC)O[C@H]1n1ccc(=O)[nH]c1=O)N(C(C)C)C(C)C]

Preparation of compound 2: To a solution of 1 (80 g, 372.8 mmol) in DMF (800 mL). Then the mixture was added Imidazole (55.7 g, 816.3 mmol), TiPSCl₂ (113.6 g, 360.5 mmol) under N₂ atmosphere. The mixture was stirred at r.t. for 4 h. Then the solution was diluted with BA, washed with water twice. The solvent was concentrated under reduced pressure to give the crude. The crude was purified by silica gel; mobile phase, PE/EA=50/1 increasing to PE/EA=1/1, the eluted product was collected at PE/EA=2/1; filtered to get 2 (131 g, 269.4 mmol, 72.2% yield) as a solid. ESI-LCMS: m/z 487.5 [M+H]⁺.

Preparation of compound 3: To a stirred mixture of 2 (131 g, 269.4 mmol) in pyridine (1.3 L) was added AgNO₃ (82.4 g, 484.9 mmol), 2,4,6-Collidine (195.6 g, 1600 mmol) and DMTrCl (136.9 g, 404.1 mmol) at room temperature under N₂ atmosphere. The resulting mixture was stirred for 16 h at 60° C. under argon atmosphere. The reaction was quenched by the addition of sat. NaHCO₃ (aq.). The resulting mixture was extracted with EtOAc. The combined organic layers were washed with brine, dried over anhydrous Na₂SO₄. After filtration, the filtrate was concentrated under reduced pressure. The residue was purified by silica gel column chromatography, to afford 3 (288.0 g crude) as an oil. ESI-LCMS: m/z 787.0 [M−H]⁻.

Preparation of compound 4: To a solution of 3 (288.0 g crude) in THF (3.0 L) and was added 1M TBAF (404 ml, 404.1 mmol). The mixture was stirred at r.t. for 3 h. LCMS showed 3 was consumed completely. H₂O was added to the mixture. Then the solution diluted with EA. The organic layer was washed with NaHCO₃ (aq.) and brine. The solvent was concentrated under reduced pressure and filtered to get a crude. The crude was purified by silica gel; mobile phase PE/EA=100/1 increasing to PE/EA=1/3, the eluted product was collected at PE/EA=1/1; filtered to get 4 (112.0 g, 205.1 mmol, 76.1% yield) as a solid. ESI-LCMS: m/z 545.3 [M−H]⁻.

Preparation of compound 5: To a stirred mixture of 4 (112.0 g, 205.1 mmol) in pyridine (1.1 L) was added Imidazole (20.9 g, 307.6 mmol), TBDPSCL (33.8 g, 225.6 mmol) at room temperature under N₂ atmosphere. The resulting mixture was stirred for 3 h at 45° C. under argon atmosphere. LCMS showed 4 was consumed completely. The reaction was quenched by the addition of sat. NaHCO₃ (aq.). The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE (0.5% TEA)/EtOAc (2:3) to afford 5 (132.8 g, 173.7 mmol, 84.7%) as an off-white solid. ESI-LCMS: m/z 783.3 [M−H]⁻; ¹H NMR (400 MHz, DMSO-d₆) δ:11.29 (d, J=2.1 Hz, 1H), 7.59-7.15 (m, 20H), 6.84-6.61 (m, 4H), 5.99 (d, J=6.9 Hz, 1H), 5.14 (dd, J=8.0, 1.9 Hz, 1H), 4.99 (d, J=5.9 Hz, 1H), 4.18 (dd, J=7.1, 5.2 Hz, 1H), 3.91 (q, J=3.1 Hz, 1H), 3.81-3.44 (m, 8H), 3.36 (dd, J=5.7, 2.0 Hz, 1H), 0.92 (s, 9H).

Preparation of compound 6: To a solution of 5 (132.8 g, 173.7 mmol) in DCM (1.3 L). The solution was added PPTS (43.6 g, 173.7 mmol), EVE (162.6 g, 2258.1 mmol). The mixture was stirred at 25° C. for 16 h. LCMS showed 5 was consumed completely. The solvent was washed with water twice and filtered to get the crude 6 (138.5 g crude).

ESI-LCMS: m/z 855.1 [M−H]⁻.

Preparation of compound 7: To a solution of 6 (138.5 g crude) in DCM (1.4 L). The solution was added TEA (228.7 g, 2258.1 mmol) and was cooled to 0° C. The mixture was added TMSOTf (386.1 g, 1737 mmol) drop wise at 0° C. under argon atmosphere. The mixture was stirred at 25° C. for 16 h. LCMS showed 6 was consumed completely. The solvent was washed with water twice and filtered to get the crude. The residue was purified by silica gel column chromatography, eluted with PE (0.5% TEA)/EtOAc (2:3) to afford 7 (112.8 g, 139.2 mmol, 80.1%) as an off-white solid. ESI-LCMS: m/z 516.9 [M−H]⁻; 1H NMR (400 MHz, DMSO-d₆) &: 11.47 (s, 1H), 7.60-7.30 (m, 13H), 7.30-7.12 (m, 7H), 6.92-6.68 (m, 4H), 6.23-5.96 (m, 2H), 5.43-5.14 (m, 1H), 4.39 (dd, J=8.0, 4.8 Hz, 1H), 4.12-3.81 (m, 3H), 3.71 (s, 7H), 3.48 (dd, J=11.1, 3.4 Hz, 1H), 3.06 (d, J=4.8 Hz, 1H), 0.93 (s, 9H).

Preparation of compound 8: To a solution of 1M Et₂Zn (556.8 ml, 556.8 mmol) and D1M (149.1 g, 556.8 mmol) in DCM (1.1 L) was stirred at 0° C. for 10 min. The mixture was added 7 (112.8 g, 139.2 mmol) drop wise under argon atmosphere. The mixture was stirred at 25° C. for 6 h. NH₄Cl (aq.) was added to the mixture. Then filtered and the solution diluted with EA. The organic layer was washed with water and brine. The organic layer was concentrated under reduced pressure and filtered to get a crude. The crude was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm, filtered to get 8 (25.0 g, 47.9 mmol, 34.4% yield) as a solid. ESI-LCMS: m/z 523.5 [M+H]⁺; ¹H NMR (400 MHz, DMSO-d₆) δ 11.37 (d, J=2.2 Hz, 1H), 7.71 (d, J=8.1 Hz, 1H), 7.63 (ddd, J=7.7, 5.9, 1.8 Hz, 4H), 7.55-7.34 (m, 7H), 5.75 (d, J=4.6 Hz, 1H), 5.58 (d, J=5.9 Hz, 1H), 5.29 (dd, J=8.1, 2.1 Hz, 1H), 4.31-4.21 (m, 1H), 4.12-3.96 (m, 3H), 3.93 (dd, J=11.6, 2.8 Hz, 1H), 3.74 (dd, J=11.5, 2.6 Hz, 1H), 3.54 (tt, J=6.0, 3.1 Hz, 1H), 1.04 (s, 10H), 0.64-0.26 (m, 4H).

Preparation of compound 9: To a solution of 8 (25.0 g, 47.9 mmol) in THF (250 mL) and added 1M TBAF (72 ml, 71.8 mmol). The mixture was stirred at r.t. for 6 h. LCMS showed 8 was consumed completely. H₂O was added to the mixture. Then filtered and the solution diluted with EA. The organic layer was washed with NaHCO₃ (aq.) and brine. The solvent was concentrated under reduced pressure and filtered to get a crude. The crude was purified by silica gel; mobile phase, DCM/MeOH=100/1 increasing to DCM/MeOH=20/1, the eluted product was collected at DCM/MeOH=30/1; filtered to get 9 (13.0 g, 45.7 mmol, 95.5% yield) as a solid. ESI-LCMS: m/z 285.0 [M+H]⁺.

Preparation of compound 10: To a stirred mixture of 9 (13.0 g, 45.7 mmol) in pyridine (130 mL) was added DMTrCl (23.2 g, 68.5 mmol) at room temperature under N₂ atmosphere. The resulting mixture was stirred for 3 h at room temperature under argon atmosphere. The reaction was quenched by the addition of sat. NaHCO₃ (aq.). The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE (0.5% TEA)/EtOAc (2:3) to afford 10 (18.5 g, 31.5 mmol, 69.0%) as an off-white solid. ESI-LCMS: m/z 585.3 [M−H]⁻; ¹H NMR (400 MHz, DMSO-d₆) δ:11.37 (s, 1H), 7.77 (d, J=8.1 Hz, 1H), 7.46-7.15 (m, 9H), 7.01-6.76 (m, 4H), 5.70 (d, J=4.1 Hz, 1H), 5.55 (d, J=5.8 Hz, 1H), 5.34 (d, J=8.1 Hz, 1H), 4.29 (q, J=4.9 Hz, 1H), 3.75 (s, 6H), 3.49 (dq, J=8.9, 4.3, 3.6 Hz, 1H), 3.35-3.11 (m, 2H), 1.17 (t, J=7.1 Hz, 2H), 0.68-0.26 (m, 4H).

Preparation of compound 344: To a solution of 10 (5.5 g, 9.4 mmol) in DCM (30 mL) was added DCI (942 mg, 7.9 mmol) and CEP[N(iPr)₂]₂ (3.4 g, 11.3 mmol) under N2. The mixture was stirred at 20° C. for 2.5 h. LCMS showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 344 (5.2 g, 6.6 mmol, 70.3% yield) as a white solid. ESI-LCMS: m/z 787.4 [M+H]⁺; ¹H NMR (400 MHz, DMSO-d₆) δ: 11.40 (s, 1H), 7.77 (dd, J=8.1, 7.0 Hz, 1H), 7.43-7.16 (m, 9H), 7.00-6.79 (m, 4H), 5.85 (dd, J=11.9, 4.7 Hz, 1H), 5.40 (t, J=7.8 Hz, 1H), 4.67-4.46 (m, 1H), 4.17-3.95 (m, 2H), 3.86-3.44 (m, 10H), 3.41-3.33 (m, 1H), 3.23 (ddd, J=11.1, 7.9, 3.8 Hz, 1H), 2.84-2.69 (m, 2H), 1.28-0.95 (m, 12H), 0.62-0.26 (m, 4H). ³¹P NMR (162 MHz, DMSO-d₆) δ: 149.98, 149.83.

### Example 49—Preparation of Compound 345

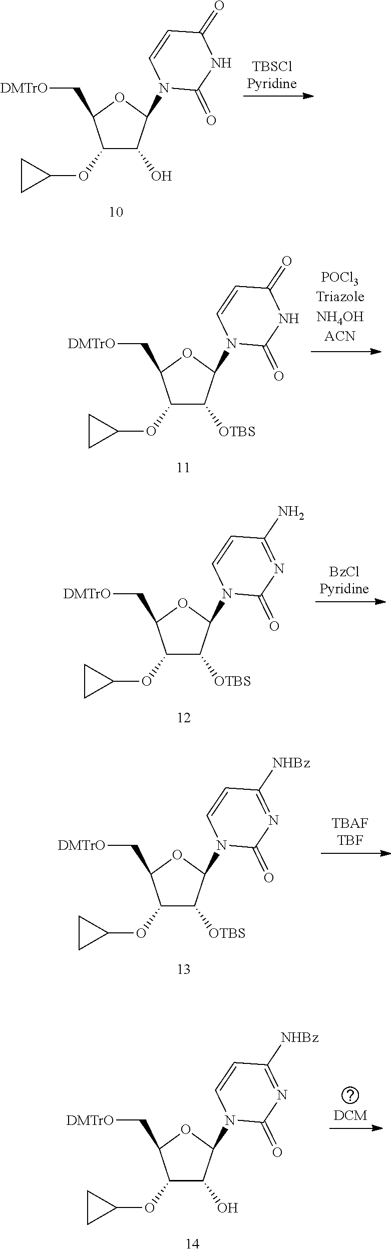

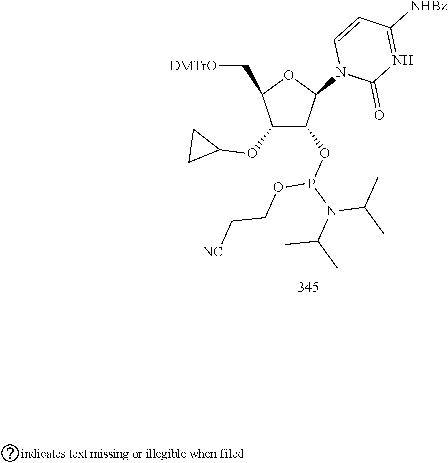

Preparation of compound 11: To a solution of 10 (10.0 g, 17.1 mmol) in dry DMF (100 mL) was added Imidazole (2.9 g, 42.7 mmol) and TBSCl (7.7 g, 51.3 mmol) at room temperature. After the solution was stirred at r.t. for 3.5 h, water (500 mL) was added, and the resulting mixture was extracted with EA (300 mL). The combined organic layer was washed with water and brine, dried over Na₂SO₄, and concentrated to give the crude 11 (12.0 g crude) as an oil which was used directly for next step. ESI-LCMS: m/z 699.3 [M−H]⁻.

Preparation of compound 12: To a solution of 11 (12.0 g crude) in dry ACN (120 ml) was added triazole (9.4 g, 136.8 mmol) and TEA (27.6 g, 273.6 mmol). Then POCl₃ (5.2 g, 34.2 mmol) was added to the mixture at 0″° C. The reaction mixture was stirred at r.t. under N₂ atmosphere for 3 h. TLC (PE:EA=2:1) showed 11 was consumed completely. 38% NH₄OH (50 mL) was added to the mixture. The reaction mixture was then stirred at r.t. for 16 h. After addition of water (800 mL), the resulting mixture was extracted with EA (250 mL). The organic layer was washed with water and brine, dried over Na₂SO₄, and concentrated under reduced pressure and the residue was purified by silica gel column chromatography (SiO₂, PE/EA=5:1˜1:2) to give 12 (13.0 g crude) as an oil which was used directly for next step. ESI-LCMS: m/z 698.4 [M−H]⁻.

Preparation of compound 13: To a solution of 12 (13.0 g crude) in pyridine (130 mL) was added BzCl (2.9 g, 20.5 mmol) at 0° C. After the solution was stirred for 2.5 h, LCMS showed 12 was consumed completely. Water (800 mL) was added, and the resulting mixture was extracted with EA (300 mL). The combined organic layer was washed with water and brine, dried over Na₂SO₄, and concentrated to give the crude 13 (14.0 g crude) which was used directly for next step. ESI-LCMS: m/z 802.3 [M−H]⁻.

Preparation of compound 14: To a solution of 13 (14.0 g crude) in THF (140 mL) and was added 1M TBAF (26 ml, 25.6 mmol). The mixture was stirred at r.t. for 3 h. LCMS showed 13 was consumed completely. H₂O was added to the mixture. Then the solution diluted with EA. The organic layer was washed with NaHCO₃ (aq.) and brine. The solvent was concentrated under reduced pressure and filtered to get a crude. The crude was purified by silica gel; mobile phase PE/EA=100/1 increasing to PE/EA=1/3, the eluted product was collected at PE/EA=1/1; filtered to get 14 (6 g, 8.7 mmol, 50.9% yield over 4 steps) as a solid. ESI-LCMS: m/z 688.0 [M−H]⁻. ¹H NMR (400 MHz, DMSO-d₆) δ: 11.28 (s, 1H), 8.47 (d, J=7.5 Hz, 1H), 8.01 (dd, J=7.9, 1.4 Hz, 2H), 7.69-7.07 (m, 14H), 7.05-6.79 (m, 4H), 5.87-5.67 (m, 2H), 4.38-4.27 (m, 1H), 4.14 (qd, J=8.4, 4.9 Hz, 2H), 3.76 (s, 6H), 3.52-3.36 (m, 2H), 3.26 (dd, J=11.3, 2.9 Hz, 1H), 0.62-0.29 (m, 4H).

Preparation of compound 345: To a solution of 14 (6 g, 8.7 mmol) in DCM (60 mL) was added DCI (873 mg, 7.4 mmol) and CEP[N(iPr)₂]₂ (3.4 g, 11.3 mmol) under N₂. The mixture was stirred at 20° C. for 2.5 h. LCMS showed 9 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 345 (6.8 g, 7.6 mmol, 87.9% yield) as a white solid. ESI-LCMS: m/z 890.3 [M+H]⁺; ¹H NMR (400 MHz, DMSO-d₆) δ: 11.29 (s, 1H), 8.46 (d, J=7.5 Hz, 1H), 8.01 (d, J=7.6 Hz, 2H), 7.69-7.11 (m, 13H), 6.93 (d, J=8.5 Hz, 4H), 6.01-5.92 (m, 1H), 4.64 (dddd, J=21.1, 11.1, 4.3, 2.2 Hz, 1H), 4.32-4.06 (m, 2H), 3.98-3.38 (m, 12H), 3.35-3.22 (m, 1H), 2.80 (h, J=5.6 Hz, 2H), 1.22-1.10 (m, 12H), 0.66-0.23 (m, 4H). ³¹P NMR (162 MHz, DMSO-d₆) δ: 150.67, 149.47.

### Example 50—Preparation of Compound 346

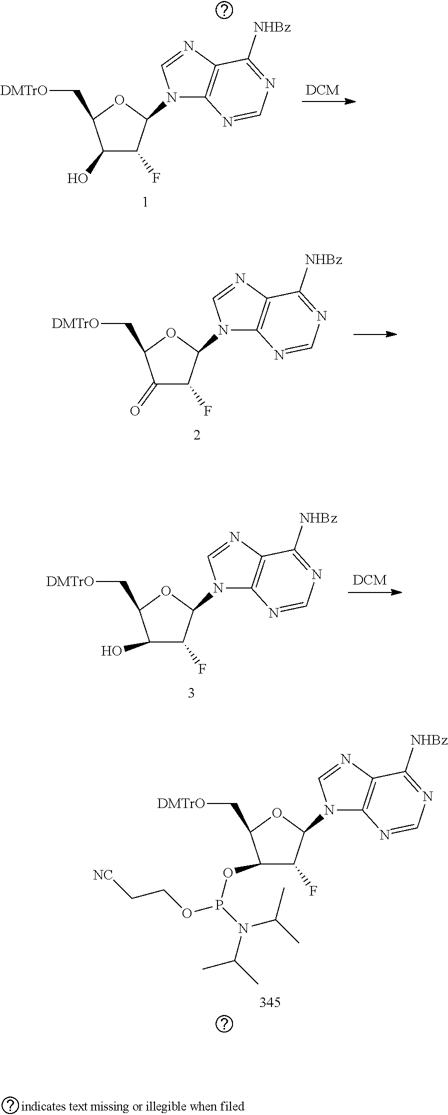

Preparation of compound 2: To a solution of chromium (VI) oxide (6.7 g, 66.7 mmol) in DCM (200 mL), and was added pyridine (8.4 g, 106.8 mmol). acetic anhydride (6.8 g, 66.7 mmol) at 0° C. under N₂ atmosphere. The mixture was stirred until the mixture was cleared. Then the solution was added 1 (18.0 g, 26.7 mmol), stirred at r.t. for 2 h. TLC showed the reaction was consumed. The mixture was diluted with EA. The organic layer was filtered and concentrated to give the crude 2 (18.3 g crude) as a solid. ESI-LCMS: m/z 674.3 [M+H]⁺.

Preparation of compound 3: To a solution of 2 (18.3 g crude, 26.7 mmol) in THF (200 mL). The solution was added borane tert-butylamine complex (4.6 g, 53.4 mmol). The mixture was stirred at 0° C. for 2 h. LCMS showed 2 was consumed completely. The mixture was diluted with EA, and was washed with water twice and the organic layer was concentrated to give the crude. The crude was purified by Flash-Prep-HPLC with the following conditions (IntelFlash-1): Column, C18 silica gel; mobile phase, CH₃CN/H₂O (0.5% NHAHCO₃)=3/7 increasing to CH₃CN/H₂O (0.5% NH₄HCO₃)=4/1 within 25 min, the eluted product was collected at CH₃CN/H₂O (0.5% NH₄HCO₃)=56/44; Detector, UV 254 nm. This resulted in 3 (5.3 g, 7.8 mmol) as a white solid. ESI-LCMS: m/z 676.3 [M+H]⁺. ¹H NMR (400 MHz, DMSO-d₆) δ 11.25 (s, 1H), 8.78 (s, 1H), 8.26 (s, 1H), 8.08-8.01 (m, 2H), 7.72-7.50 (m, 3H), 7.50-7.13 (m, 9H), 6.94-6.80 (m, 4H), 6.45 (d, J=20.3 Hz, 1H), 5.97 (d, J=4.6 Hz, 1H), 5.46 (d, J=48.8 Hz, 1H), 4.57-4.32 (m, 2H), 3.72 (d, J=2.8 Hz, 6H), 3.48 (dd, J=10.4, 7.8 Hz, TH), 3.30 (t, J=5.1 Hz, 1H).

Preparation of compound 346: To a solution of 3 (5.3 g, 7.8 mmol) in DCM (50 mL) was added DCI (787.0 mg, 6.7 mmol) and CEP[N(iPr)₂]₂ (2.8 g, 9.4 mmol) under N₂. The mixture was stirred at 20° C. for 2.5 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C15 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 346 (5.0 g, 5.7 mmol, 72.6% yield) as a white solid. ESI-LCMS: m/z 858.4 [M+H]⁺; ¹H NMR (400 MHz, DMSO-d₆) δ 11.25 (s, 1H), 8.78 (d, J=8.6 Hz, 1H), 8.20 (s, 1H), 8.04 (dt, J=8.6, 1.3 Hz, 2H), 7.56 (td, J=7.5, 7.0, 1.5 Hz, 3H), 7.51-7.36 (m, 2H), 7.34-7.18 (m, 6H), 6.87 (ddt, J=9.1, 6.9, 2.8 Hz, 4H), 6.53 (dd, J=19.3, 1.6 Hz, 1H), 5.74 (d, J=48.7 Hz, 1H), 4.89-4.51 (m, 2H), 4.03 (q, J=7.1 Hz, 1H), 3.72 (dd, J=2.7, 1.6 Hz, 6H), 3.65-3.44 (m, 3H), 3.31-3.09 (m, 2H), 2.66-2.50 (m, 3H), 1.99 (s, 2H), 1.17 (t, J=7.1 Hz, 2H), 1.00 (dd, J=19.3, 6.7 Hz, 5H), 0.78 (dd, J=10.4, 6.7 Hz, SH). ¹⁹F NMR (376 MHz, DMSO-d₆) δ −186.80. ³¹P NMR (162 MHz, DMSO-d₆) δ 150.94, 148.61.

### Example 51—Preparation of Compound 347

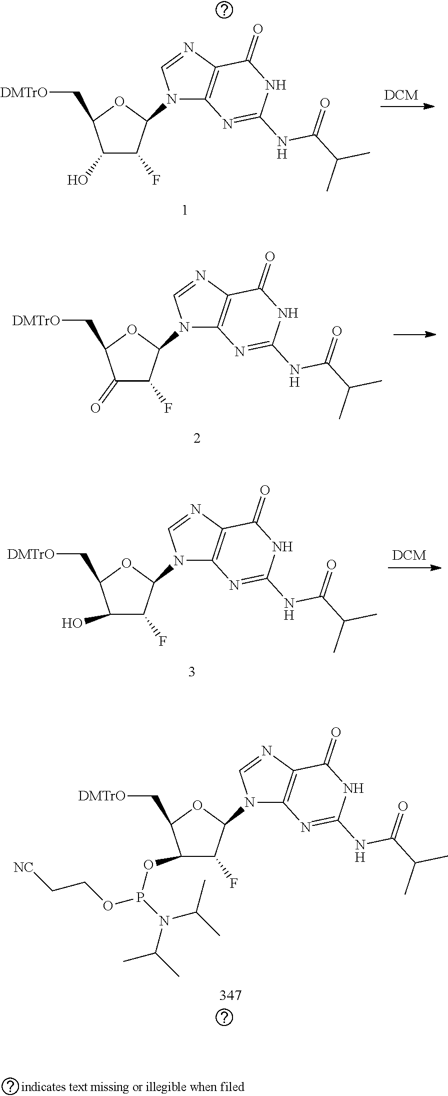

Preparation of compound 2: To a solution of chromium (VI) oxide (6.8 g, 68.5 mmol) in DCM (200 mL), and was added pyridine (8.6 g, 109.5 mmol), acetic anhydride (6.9 g, 68.42 mmol) at 0° C. under N₂ atmosphere. The mixture was stirred until the mixture was cleared. Then the solution was added 1 (18.0 g, 27.4 mmol), stirred at r.t. for 2 h. TLC showed the reaction was consumed. The mixture was diluted with EA. The organic layer was filtered and concentrated to give the crude 2 (18.5 g crude) as a solid. ESI-LCMS: m/z 656 [M+H]⁺.

Preparation of compound 3: To a solution of 2 (18.5 g crude, 27.4 mmol) in THF (200 mL). The solution was added borane tert-butylamine complex (4.7 g, 54.8 mmol). The mixture was stirred at 0° C. for 2 h. LCMS showed 2 was consumed completely. The mixture was diluted with EA, and was washed with water twice and the organic layer was concentrated to give the crude. The crude was purified by Flash-Prep-HPLC with the following conditions (IntelFlash-1): Column, C18 silica gel; mobile phase, CH₃CN/H₂O (0.5% NH₄HCO₃)=3/7 increasing to CH₃CN/H₂O (0.5% NH₄HCO₃)=4/1 within 25 min, the eluted product was collected at CH₃CN/H₂O (0.5% NH₄HCO₃)=62/38; Detector, UV 254 nm. This resulted in 3 (4.3 g, 6.5 mmol) as a white solid. ESI-LCMS: m/z 658 [M+H]⁺. ¹H NMR (400 MHz, DMSO-d₆) δ 12.15 (s, 1H), 11.76 (s, 1H), 7.80 (s, 1H), 7.56-7.13 (m, 9H), 6.86 (dd, J=8.8, 7.2 Hz, 4H), 6.14 (s, OH), 5.89 (d, J=4.1 Hz, 1H), 5.76 (s, 1H), 5.26 (s, OH), 4.50-4.34 (m, 2H), 3.76-3.71 (m, 6H), 3.47 (dd, J=10.4, 7.9 Hz, 1H), 3.29 (dd, J=10.4, 3.1 Hz, 1H), 2.80 (p. J=6.8 Hz, 1H), 1.27-1.09 (m, 7H).

Preparation of compound 347: To a solution of 3 (4.3 g, 6.5 mmol) in DCM (40 mL) was added DCI (652.5 mg, 5.5 mmol) and CEP[N(iPr)₂]₂ (2.4 g, 7.8 mmol) under N₂. The mixture was stirred at 20° C. for 2.5 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 347 (3.6 g, 4.1 mmol, 63.1% yield) as a white solid. ESI-LCMS: m/z 876.4 [M+H]⁺; ¹H NMR (400 MHz, DMSO-d₆) δ 12.13 (s, 1H), 11.77 (s, 1H), 7.70 (d, J=13.6 Hz, 1H), 7.49-7.20 (m, 9H), 6.87 (dt, J=9.0, 4.7 Hz, 4H), 6.24 (dd, J=19.8, 3.2 Hz, 1H), 5.82-5.35 (m, 1H), 4.73-4.43 (m, 2H), 3.74 (s, 6H), 3.63 (did, J=15.1, 7.5, 4.6 Hz, 1H), 3.47 (dddd, J=16.8, 12.4, 7.2, 3.4 Hz, 2H), 3.27-3.14 (m, 2H), 2.93-2.53 (m, 3H), 1.38-0.91 (m, 13H), 0.81 (t, J=6.5 Hz, 6H). ³¹P NMR (162 MHz, DMSO-d₆) δ 151.24, 148.05.

### Example 52—Preparation of Compound 348

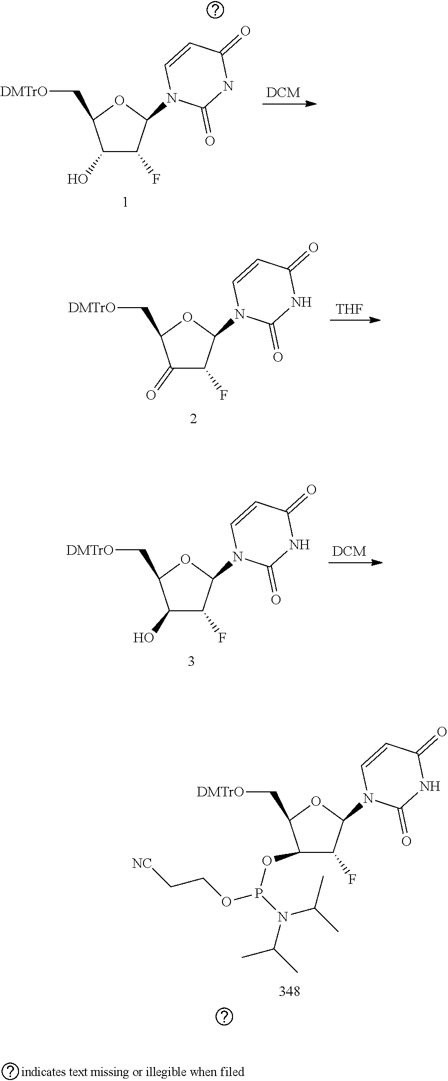

Preparation of compound 2: To a solution of chromium (VI) oxide (8.2 g, 82.1 mmol) in DCM (200 mL), and was added pyridine (10.4 g, 131.3 mmol), acetic anhydride (8.3 g, 82.1 mmol) at 0° C. under N; atmosphere. The mixture was stirred until the mixture was cleared. Then the solution was added 1 (18.0 g, 32.8 mmol), stirred at r.t. for 2 hr. TLC showed the reaction was consumed. The mixture was diluted with EA. The organic layer was filtered and concentrated to give the crude 2 (19.5 g crude) as a solid. ESI-LCMS: m/z 547.2 [M+H]⁺.

Preparation of compound 3: To a solution of 2 (19.5 g crude, 32.8 mmol) in THF (200 mL). The solution was added borane tert-butylamine complex (5.7 g, 65.6 mmol). The mixture was stirred at 0° C. for 1 h. LCMS showed 2 was consumed completely. The mixture was diluted with EA, and was washed with water twice and the organic layer was concentrated to give the crude. The crude was purified by Flash-Prep-HPLC with the following conditions (IntelFlash-1): Column, C18 silica gel; mobile phase, CH₃CN/H₂O (0.5% NH₄HCO₃)=3/7 increasing to CH₃CN/H₂O (0.5% NH₄HCO₃)=4/1 within 25 min, the eluted product was collected at CH₃CN/H₂O (0.5% NH₄HCO₃)=52/48; Detector, UV 254 nm. This resulted in 3 (5.1 g, 9.3 mmol) as a white solid. ESI-LCMS: m/z 549.3 [M+H]⁺. ¹H NMR (400 MHz, DMSO-d₆) δ 11.44 (s, 1H), 7.44 (dd, J=8.4, 1.6 Hz, 3H), 7.36-7.19 (m, 7H), 6.95-6.85 (m, 4H), 5.99 (d, J=21.6 Hz, 1H), 5.55 (d, J=8.1 Hz, 1H), 4.99 (d, J=48.7 Hz, 1H), 4.38-4.30 (m, 1H), 4.24 (dt, J=9.7, 3.6 Hz, 1H), 3.74 (s, 6H), 3.43 (dd, J=10.4, 7.7 Hz, 1H), 3.29 (dd, J=10.4, 3.4 Hz, 1H).

Preparation of compound 348: To a solution of 3 (5.1 g, 9.3 mmol) in DCM (50 mL) was added DCI (933.6 mg, 7.9 mmol) and CEP[N(iPr)₂]₂ (3.4 g, 11.2 mmol) under N₂. The mixture was stirred at 25° C. for 2 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=9/1; Detector, UV 254 nm. This resulted in 348 (5.2 g, 6.9 mmol, 74.8% yield) as a white solid. ESI-LCMS: m/z 749.3 [M+H]+; ¹H NMR (400 MHz, DMSO-d₆) δ 11.46 (s, 1H), 7.52-7.17 (m, 10H), 6.89 (t, J=9.0 Hz, 4H), 6.01 (d, J=20.6 Hz, 1H), 5.48 (dd, J=18.9, 8.1 Hz, 1H), 5.15 (dd, J=48.3, 28.9 Hz, 1H), 4.60-4.41 (m, 2H), 3.74 (d, J=3.5 Hz, 6H), 3.64-3.47 (m, 2H), 3.43 (dd, J=10.6, 7.9 Hz, 1H), 3.40-3.20 (m, 3H), 2.79-2.53 (m, 2H), 1.05 (dd, J=10.8, 6.7 Hz, 6H), 0.86 (dd, J=22.5, 6.7 Hz, 6H). ³¹P NMR (162 MHz, DMSO-d₆) δ 151.29, 148.54.

### Example 53—Preparation of Compound 349

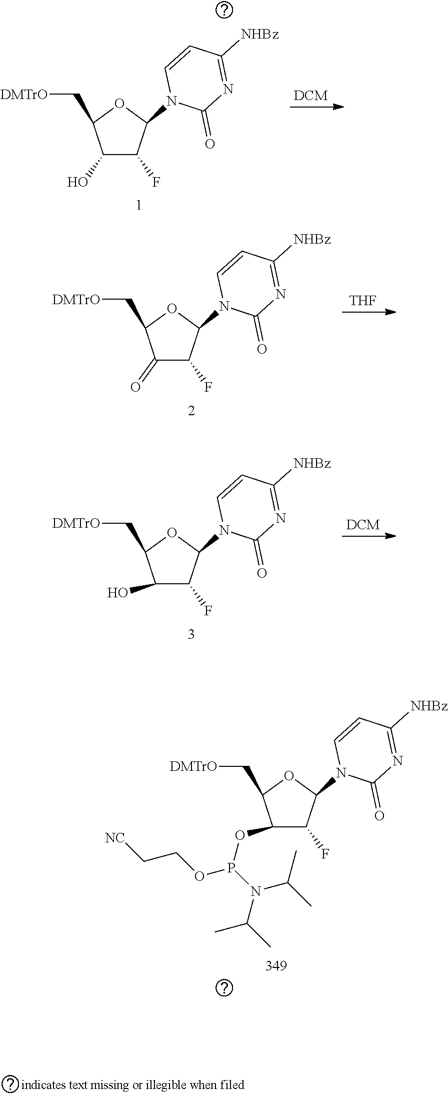

Preparation of compound 2: To a solution of Chromium (VI) oxide (6.8 g, 68.4 mmol) in DCM (200 mL), and was added pyridine (8.6 g, 109.4 mmol), Acetic anhydride (6.8 g, 68.4 mmol) at 0° C. under N₂ atmosphere. The mixture was stirred until the mixture was cleared. Then the solution was added 1 (17.8 g, 27.3 mmol), stirred at r.t. for 2 hr. TLC showed the reaction was consumed. The mixture was diluted with EA. The organic layer was filtered and concentrated to give the crude 2 (18.0 g crude) as a solid. ESI-LCMS: m/z 650.0 [M+H]⁺.

Preparation of compound 3: To a solution of 2 (18.0 g crude) in THF (180 mL). Then the solution was added NaBH4 (1.5 g, 41.0 mmol) in 0° C. The mixture was stirred at 0° C. for 1 h. LCMS showed 2 was consumed completely. Then the solution was quench with NH₄Cl (aq) and diluted with EA, washed with water twice and brine. The organic phase dried over anhydrous Na₂SO₄, filtered and concentrated under reduced pressure to get a crude. The crude was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=3/1 within 40 min, the eluted product was collected at CH₃CN/H₂O (0.5% NHAHCO₃)=3/2; Detector, UV 254 om, filtered to get 3 (5.2 g, 7.97 mmol, 29.2% yield, 99% purity) as a solid, ESI-LCMS: m/z 652.0 [M+H]+. ¹H NMR (400 MHz, DMSO-d₆) δ 11.29 (s, 1H), 8.05-7.98 (m, 2H), 7.82 (d, J=7.6 Hz, 1H), 7.67-7.58 (m, 1H), 7.59-7.44 (m, 4H), 7.40-7.21 (m, 8H), 6.99-6.88 (m, 4H), 5.98 (d, J=19.3 Hz, 1H), 5.66 (d, J=3.5 Hz, 1H), 4.99 (d, J=48.2 Hz, 1H), 4.47 (dd, J=7.8, 3.2 Hz, 1H), 4.26 (dt, J=7.5, 3.4 Hz, 1H), 3.76 (d, J=1.3 Hz, 6H), 3.52 (dd, J=10.6, 7.9 Hz, 1H), 3.39 (dd, J=10.6, 3.1 Hz, 1H).

Preparation of compound 349: To a solution of 3 (5.2 g, 7.9 mmol) in DCM (50 mL) was added DCI (799 mg, 6.7 mmol) and CEP[N(IPr)₂]₂ (3.3 g, 11.1 mmol) under N₂. The mixture was stirred at 40° C. for 4 h. LCMS showed 3 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 349 (5 g, 5.8 mmol, 73.6% yield) as a white solid. ESI-LCMS: m/z 852.1 [M+H]+; ¹H NMR (400 MHz, DMSO-d₆) δ 11.29 (d, J=12.7 Hz, 1H), 8.00 (d, J=7.6 Hz, 2H), 7.84-6.64 (m, 18H), 6.01 (dd, J=18.5, 10.1 Hz, 1H), 5.20 (t, J=48.7 Hz, 1H), 4.79-4.41 (m, 2H), 3.76 (s, 6H), 3.67-3.47 (m, 2H), 3.36-3.12 (m, 3H), 2.71-2.63 (m, 1H), 2.57 (s, 1H), 0.98 (dd, J=22.6, 6.7 Hz, 6H), 0.80 (t, J=7.4 Hz, 6H). ³¹P NMR (162 MHz, DMSO-d₆) δ 151.19, 147.94.

### Example 54—Preparation of Compound 350

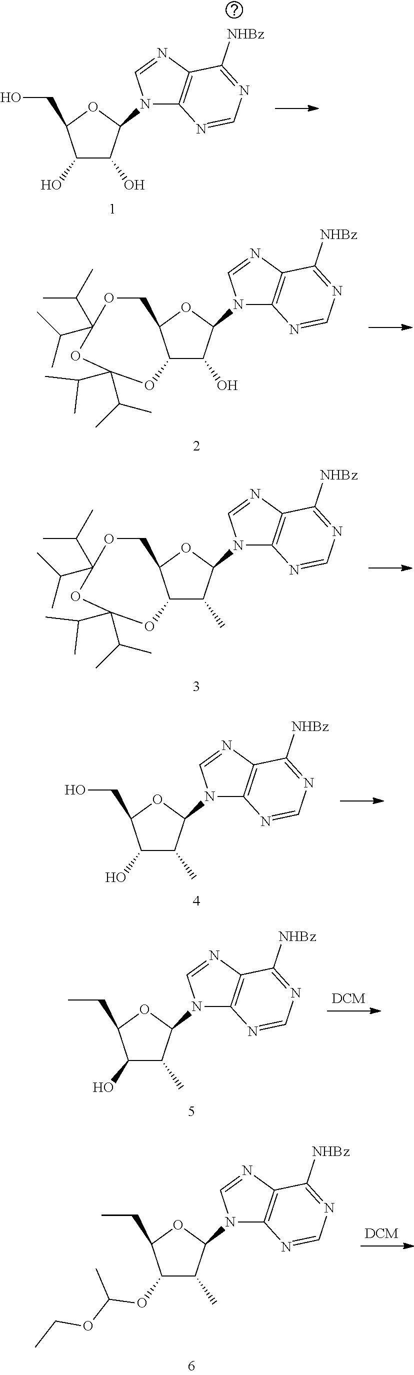

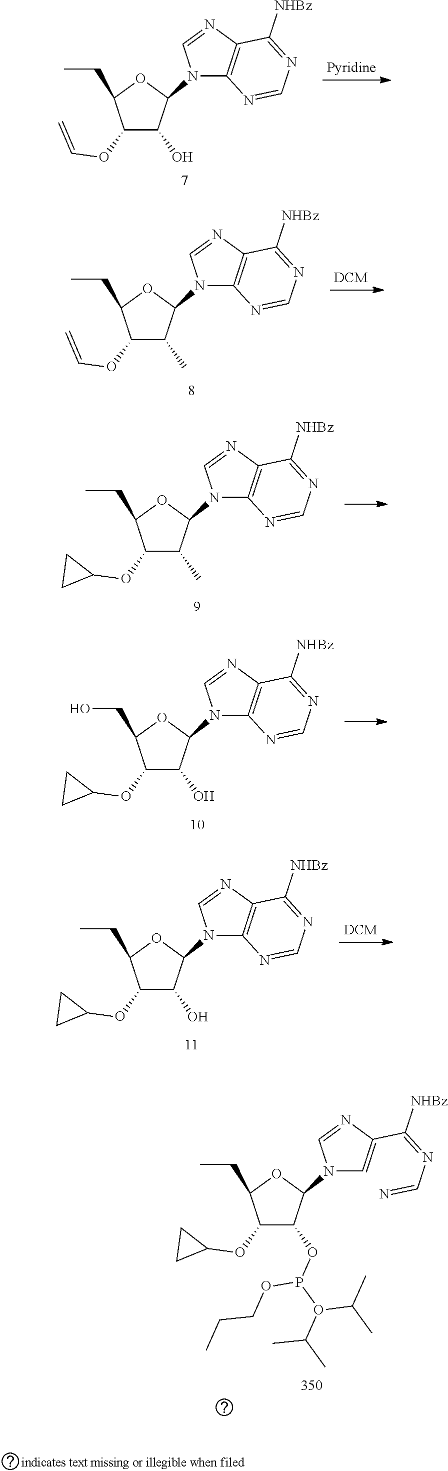

Preparation of 2: To a solution of 1 (115.0 g, 309.9 mmol) in DMF (1.1 L). Then the mixture was added imidazole (73.7 g, 371.9 mmol), TiPSCl₂ (117.1 g, 309.9 mmol) under N₂ atmosphere. The mixture was stirred at r.t. for 4 h. Then the solution was diluted with EA, washed with water twice. The solvent was concentrated under reduced pressure to give the crude. The crude was purified by silica gel column chromatograph (eluent, PE/EA=50:1˜1:1). This resulted in 2 (84.0 g, 137.0 mmol, 44.2% yield) as a solid. ESI-LCMS: m/z 614.2 [M+H]⁺.

Preparation of 3: To a stirred mixture of 2 (84 g, 137.0 mmol) in pyridine (800 mL) was added AgNO₃ (41.9 g, 246.6 mmol), 2, 4, 6-Collidine (132.6 g, 1096.0 mmol) and DMTrCl (92.8 g, 274.0 mmol) at r.t under N₂ atmosphere. The resulting mixture was stirred at 60° C. for 16 h under argon atmosphere. The reaction was quenched by the addition of sat. NaHCO₃ (aq.). The resulting mixture was extracted with EA. The combined organic layers was washed with saturated brine and dried over by Na₂SO₄. Then the solution was concentrated under reduced pressure. This resulted in crude 3 (150 g) as a brown oil. ESI-LCMS: m/z 916.4 [M+H]⁺.

Preparation of 4: To a solution of crude 3 (150 g) in THF (1.5 L) and was added 1M TBAF (225 mL, 224.9 mmol). The mixture was stirred at r.t. for 3 h. LC-MS showed 3 was consumed completely. Then the solution was added water (1.0 L), the product was extracted with EA, and was washed with water. Then washed the organic phase once with NaHCO₃ (aq.) and saturated brine and dried over by Na₂SO₄. Then the solution was concentrated under reduced pressure. This resulted was purified by silica gel column chromatograph (eluent, PE:EA=15:1˜8:1). The residue in to give 4 (65.2 g, 96.8 mmol, 70.6% yield) as a yellow solid. ESI-LCMS: m/z 674.2 [M+H]⁺; ¹H NMR (400 MHz, DMSO-d₆) δ: 11.20 (s, 1H), 8.61 (s, 1H), 8.42 (s, 1H), 8.06 (dt, J=7.0, 1.4 Hz, 2H), 7.74-7.50 (m, 3H), 7.45-7.00 (m, 9H), 6.76-6.53 (m, 4H), 6.08 (d, J=6.6 Hz, 1H), 5.10 (t, J=5.0 Hz, 2H), 4.88 (dd, J=6.6, 4.8 Hz, 1H), 3.98 (d, J=2.6 Hz, 1H), 3.66 (d, J=14.5 Hz, 6H), 3.53 (dt, J=12.0, 4.2 Hz, 1H), 3.45-3.34 (m, 2H).

Preparation of 5: To a mixture of 4 (65.2 g, 96.8 mmol) in pyridine (700 mL) was added imidazole (6.5 g, 96.8 mmol), TBDPSCL (27.9 g, 101.6 mmol) at r.t under N₂ atmosphere. The resulting mixture was stirred at 45° C. for 3 h under argon atmosphere. LC-MS showed 4 was consumed completely. The reaction was quenched by the addition of sat. NaHCO₃ (aq.). The resulting mixture was extracted with EA. The combined organic layers were washed the organic phase once with saturated brine and dried over by Na₂SO₄. Then the solution was concentrated under reduced pressure. This resulted was purified by silica gel column chromatograph (eluent, PE (0.5% TEA)/EA=100:1˜1:1). This resulted in 5 (70 g, 76.7 mmol, 79.2% yield) as a white solid. ESI-LCMS: m/z 912.1 [M+H]⁺; ¹H NMR (400 MHz, DMSO-d₆) δ: 11.15 (s, 1H), 8.45 (s, 1H), 8.31 (s, 1H), 8.14-7.97 (m, 2H), 7.74-7.03 (m, 22H), 6.76-6.48 (m, 4H), 5.90 (d, J=5.5 Hz, 1H), 5.25 (d, J=5.9 Hz, 1H), 5.04 (t, J=5.3 Hz, 1H), 4.08 (q, J=4.6 Hz, 1H), 3.79 (ddd, J=41.0, 10.5, 5.1 Hz, 2H), 3.64 (d, J=10.2 Hz, 6H), 0.88 (s, 9H).

Preparation of 6: To a solution of 5 (70.0 g, 76.7 mmol) in DCM (700 mL) was added PPTS (32.7 g, 130.4 mmol), EVE (71.8 g, 997.1 mmol) at r.t. The mixture was stirred at 25° C. for 16 h. LC-MS showed 5 was consumed completely. The residue was diluted with DCM (500 mL) and was washed with water. Then the organic phase was washed once with saturated brine and dried over by Na₂SO₄. Then the solution was concentrated under reduced pressure. The residue in to give crude 6 (76.0 g) as a white solid. ESI-LCMS: m/z 984.1 [M+H]⁺.

Preparation of 7: To a solution of 6 (76.0 g crude) was dissolved in DCM (3% DCA) (800 mL) at −5° C. The mixture was stirred at −5° C. for 0.5 h. TLC showed 6 was consumed completely. The reaction was quenched by the addition of sat. NaHCO₃ (aq.), the product was extracted with DCM. The combined organic layers were washed the organic phase once with saturated brine and dried over by Na₂SO₄. Then the solution was concentrated under reduced pressure. This resulted in crude 6a (80.0 g) as a yellow solid. To a solution of crude 6a (80.0 g) in DCM (300 mL) was added TEA (100.9 g, 997.1 mmol) at 0° C. The mixture was added TMSOTf (170.4 g, 767.2 mmol) drop wise at 0° C. under argon atmosphere. The mixture was stirred at 25° C. for 16 h. LCMS showed 6a was consumed completely. The mixture was quenching with NaHCO₃·aq, the product was extracted with DCM. Then washed the organic phase once with saturated brine and dried over by Na₂SO₄. Then the solution was concentrated under reduced pressure. This resulted was purified by silica gel column chromatograph (eluent, PE (3% TEA)/EA=100:1˜20:1). This resulted in crude 7 (85 g) as a white solid. ESI-LCMS: m/z 636.0 [M+H]⁺. ¹H NMR (400 MHz, DMSO-d₆) δ: 11.22 (s, 1H), 8.65 (s, 2H), 8.17-7.98 (m, 2H), 7.74-7.15 (m, 20H), 7.14-6.95 (m, 8H), 6.92-6.76 (m, 6H), 6.59 (dd, J=14.2, 6.7 Hz, 1H), 6.10 (d, J=5.8 Hz, 1H), 5.89 (d, J=6.0 Hz, 1H), 5.17 (q, J=5.6 Hz, 1H), 4.79 (dd, J=5.0, 3.8 Hz, 1H), 4.48 (dd, J=14.1, 1.9 Hz, 1H), 4.26 (q, J=4.5 Hz, 1H), 4.13 (dd, J=6.6, 1.9 Hz, 1H), 3.82 (dd, J=11.4, 4.7 Hz, 1H), 3.72 (s, 10H), 0.98 (s, 10H).

Preparation of 8: To a solution of crude 7 (85 g) in DMF (850 mL) was added imidazole (26.1 g, 383.5 mmol), TBSCl (23.0 g, 153.4 mmol) at r.t under N₂ atmosphere. The mixture was stirred at r.t. for 4 h. Then the solution was diluted with EA, washed with water twice. The solvent was concentrated under reduced pressure to give the crude. The crude was purified by silica gel; mobile phase, PE/EA=50/1 increasing to PE/EA=1/1, the eluted product was collected at PE/EA=3/1; Detector, UV 254 nm. This resulted in 8 (32 g, 42.7 mmol, 55.7% yield over 4 steps) as a white solid. ESI-LCMS: m/z 750.1 [M+H]⁺.

Preparation of 9: To a solution of D1M (45.7 g, 170.8 mmol) in DCM (300 mL) was added 1M Et₂Zn (170 ml, 170.8 mmol) at 0° C. The mixture was stirred at 0° C. for 10 min. The mixture was added 8 (32.0 g, 42.7 mmol) drop wise at 0° C. under argon atmosphere. The mixture was stirred at 25° C. for 6 h. LC-MS show 8 was completely consumed. The mixture was quenching with NH₄Cl·aq, the product was extracted with DCM. Then the organic phase was washed with once with saturated brine and dried over by Na₂SO₄. Then the solution was concentrated under reduced pressure. This resulted was purified by Flash-Prep-HPLC with the following conditions (IntelFlash-1): Column, C18 silica gel; mobile phase, CH₃CN/H₂O (0.05% NH₄HCO₃)=1/1 increasing to CH₃CN/H₂O (0.05% NH₄HCO₃)=1/0 within 25 min, the eluted product was collected at CH₃CN/H₂O (0.05% NH₄HCO₃)=1/0; Detector, UV 254 nm. This resulted in 9 (8.5 g crude) as a solid. ESI-LCMS: m/z 764.1 [M+H]⁺;

Preparation of 10: To a solution of crude 9 (8.5 g) in THF (80 mL) and added 1M TBAF (13.2 ml, 13.2 mmol). The mixture was stirred at r.t. for 3 h. LC-MS showed 9 was consumed completely. The reaction was quenched by water, the product was extracted with DCM. Then the organic phase was washed with once with saturated brine and dried over by Na₂SO₄. Then the solution was concentrated under reduced pressure. This resulted was purified by silica gel; mobile phase, DCM/MeOH=100/1 increasing to DCM/MeOH=20/1, the eluted product was collected at DCM/MeOH=30/1; Detector, UV 254 nm. This resulted in 10 (2.0 g, 4.8 mmol) as a white solid. ESI-LCMS: m/z 412.1 [M+H]⁺. ¹H NMR (400 MHz, DMSO-d₆) δ: 11.22 (s, 1H), 8.75 (d, J=12.1 Hz, 2H), 8.05 (dt, J=7.2, 1.4 Hz, 2H), 7.72-7.61 (m, 1H), 7.56 (dd, J=8.2, 6.8 Hz, 2H), 6.03 (d, J=5.8 Hz, 1H), 5.66 (d, J=6.3 Hz, 1H), 5.21 (t, J=5.6 Hz, 1H), 4.82 (q, J=5.7 Hz, 1H), 4.19-3.96 (m, 2H), 3.80-3.48 (m, 3H), 0.72-0.33 (m, 4H).

Preparation of 11: To a mixture of 10 (2.0 g, 4.8 mmol) in pyridine (20 mL) was added DMTrCl (1.9 g, 5.7 mmol) at r.t under N₂ atmosphere. The resulting mixture was stirred at r.t for 3 h under argon atmosphere. The reaction was quenched by the addition of sat. NaHCO₃ (aq.). The resulting mixture was extracted with EA. Then the organic phase was washed with once with saturated brine and dried over by Na₂SO₄. Then the solution was concentrated under reduced pressure. This resulted was purified by silica gel column chromatography (eluted, PE (0.5% TEA)/EA=2:3). This resulted in 11 (2.1 g, 2.9 mmol, 60.4% yield) as an off-white solid. ESI-LCMS: m/z 714.2 [M+H]⁺; ¹H NMR (400 MHz, DMSO-d₆) δ: 11.24 (s, 1H), 8.65 (d, J=26.4 Hz, 2H), 8.14-7.97 (m, 2H), 7.71-7.46 (m, 3H), 7.46-7.16 (m, 9H), 6.93-6.79 (m, 4H), 6.03 (d, J=5.1 Hz, 1H), 5.80-5.63 (m, 1H), 5.01 (q, J=5.4 Hz, 1H), 4.29 (t, J=4.9 Hz, 1H), 4.16 (q, J=4.6 Hz, 1H), 3.73 (s, 6H), 3.59 (tt, J=5.8, 3.3 Hz, 1H), 3.40-3.15 (m, 2H), 0.71-0.37 (m, 4H).

Preparation of 350: To a solution of 11 (2.1 g, 2.9 mmol) in DCM (20 mL) was added DCI (381 mg, 3.22 mmol) and CEP[N(iPr)₂]₂ (1.4 g, 4.55 mmol) under N₂. The mixture was stirred at 20° C. for 2.5 h. LC-MS showed 11 was consumed completely. The product was extracted with DCM, The organic layer was washed with H₂O and brine. Then the solution was concentrated under reduced pressure and the residue was purified by Flash-Prep-HPLC with the following conditions (IntelFlash-1): Column, C18 silica gel; mobile phase, CH₃CN/H₂O (0.5% NH₄HCO₃)=1/3 increasing to CH₃CN/H₂O (0.5% NH₄HCO₃)=1/0 within 25 min, the eluted product was collected at CH₃CN/H₂O (0.5% NH₄HCO₃)=1/0; Detector, UV 254 nm. This resulted in 350 (2.1 g, 2.3 mmol, 79.3% yield, 98.0% purity) as a white solid. ESI-LCMS: m/z 914.2 [M+H]⁺; ¹H NMR (400 MHz, DMSO-d₆) δ: 11.22 (s, 1H), 8.74-8.34 (m, 2H), 8.16-7.96 (m, 2H), 7.74-7.47 (m, 3H), 7.42-7.18 (m, 8H), 6.92-6.77 (m, 4H), 6.19 (dd, J=21.5, 5.4 Hz, 1H), 5.31 (ddt, J=18.1, 10.3, 5.2 Hz, 1H), 4.43 (t, J=4.6 Hz, 1H), 4.25 (dq, J=30.8, 4.6 Hz, 1H), 3.86-3.37 (m, 11H), 3.25 (dt, J=10.5, 4.3 Hz, 1H), 2.67 (dt, J=70.0, 5.9 Hz, 2H), 1.28-0.86 (m, 12H), 0.75-0.39 (m, 4H). ³¹P NMR (162 MHz, DMSO-d₆) δ: 149.91, 149.68.

### Example 55—Preparation of 3′OCyp Coupling Sugar

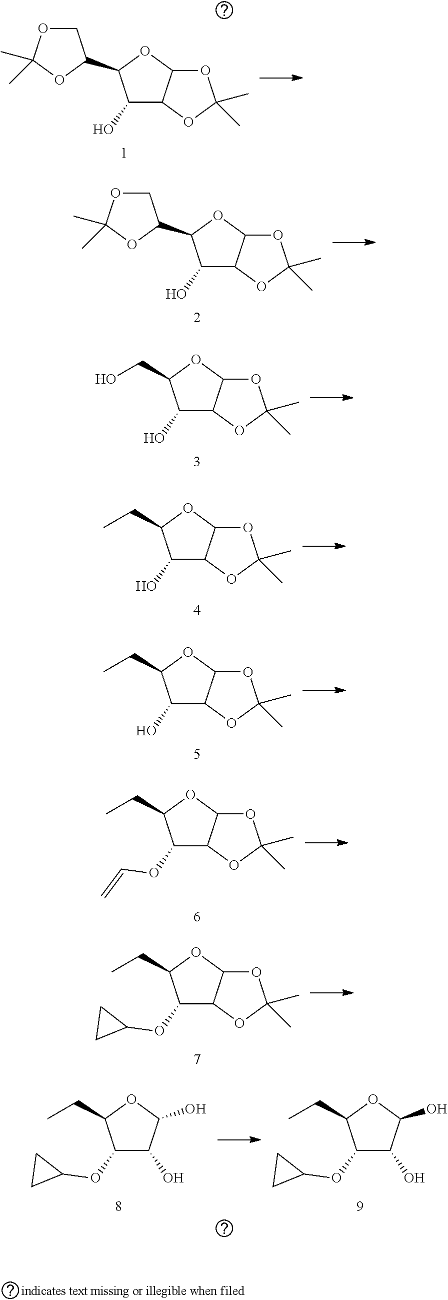

Preparation of 2: To a solution of 1 (350.0 g, 1.3 mol) in pyridine (3.0 L) was added BzCl (287 g, 2.02 mol) at 0° C. The mixture solution was stirred at 25° C. for 2 h. LC-MS and TLC show SM was completely consumed. The mixture solution was added water, and the product was extracted with EA. And the combined organic phase was washed with water and saturated brine and dried over by Na₂SO₄. Then the solution was concentrated under reduced pressure. This resulted in crude 2 (550.0 g) as a clear oil without further purified and used directly for the next step. ESI-LCMS: m/z 365.2 [M+H]⁻.

Preparation of 3: To a solution of 2 (80.0 g, 219.0 mmol) in EA (1.6 L) was added H₅IO₆ (60.0 g, 263.0 mmol) at 0° C. The mixture solution was stirred at 25° C. for 2 h. The mixture solution was filtered and the filtrate was concentrated under reduced pressure. And the resulted in EtOH (2.0 L) was added NaBH₄ (18.3 g, 480.0 mmol) at 0° C. The mixture solution was stirred at 25° C. for 2 h. LC-MS and TLC show SM was completely consumed. The mixture solution was filtered and the filtrate was concentrated under reduced pressure. The resulted was dissolved with EA, the organic phase was washed with water and saturated brine and dried over by Na₂SO₄. Then the solution was concentrated under reduced pressure. The residue was to give crude 3 (70.0 g) as a clear oil without further purified and used directly for the next step. ESI-LCMS: m/z 295.2 [M+H]⁻. ¹H NMR (400 MHz, DMSO-d₆) δ 8.00-7.92 (m, 2H), 7.75-7.62 (m, 1H), 7.61-7.49 (m, 2H), 5.91-5.85 (m, 1H), 4.95-4.80 (m, 3H), 4.25-4.17 (m, 1H), 3.72-3.51 (m, 2H), 1.43 (s, 3H), 1.26 (s, 3H).

Preparation of 4: To a solution of 3 (70.0 g, 237.0 mmol) in pyridine (700 mL) was added imidazole (32.2 g, 474.0 mmol) and TBDPSCl (98.0 g, 356.0 mmol) at 0° C. The mixture solution was stirred at 25° C. for 2 h. LC-MS show 3 was completely consumed. Then the mixture solution was added water, and the product was extracted with EA. And the organic phase was washed with water and saturated brine and dried over by Na₂SO₄. Then the solution was concentrated under reduced pressure. This resulted in crude 4 (120.0 g) as a clear oil without further purified and used directly for the next step. ESI-LCMS: m/z 555.5 [M+Na].

Preparation of 5: To a solution of 4 (120.0 g, 225.0 mmol) was dissolved in 33% C₃NH₂ in MeOH (1.2 L) at r.t. The mixture solution was stirred at 25° C. for 5 h. TLC and LC-MS show 4 was completely consumed. Then the solution was removed under reduced pressure. The residue was purified by silica gel column chromatography (eluent, PE:EA=20:1˜8:1). This resulted in 5 (45.0 g, 105.0 mmol, 50.0% yield over 4 steps) as a clear oil. ESI-LCMS: m/z 446.4 [M+H₂O]⁻. ¹H NMR (400 MHz, CDCl₃-d) δ 7.77-7.62 (m, 4H), 7.48-7.32 (m, 6H), 5.88-5.80 (m, 1H), 4.64-4.55 (m, 1H), 4.19-4.07 (m, 1H), 4.00-3.80 (m, 2H), 2.35-2.25 (m, 1H), 1.55 (s, 3H), 1.38 (s, 3H), 1.04 (s, 9H).

Preparation of 6: To a solution of 5 (45.0 g, 105.0 mmol) in BVE (210.0 g, 2.1 mol) was added 4, 7-dipenyl phenanthroline (1.7 g, 5.3 mmol) and Pd (TFA) 2 (1.7 g, 5.3 mmol) and TEA (10.6 g, 105.0 mmol) at r.t. The mixture was stirred at 25° C. for 20 min. Then the mixture was stirred at 75° C. for 15 h. LC-MS show 30% of 6 was remained. Then the solution was added water (1.0 L), the product was extracted with EA, and was washed with water. Then washed the organic phase once with saturated brine and dried over by Na₂SO₄. Then the solution was concentrated under reduced pressure. This resulted was purified by silica gel column chromatograph (eluent, PE:EA=15:1˜8:1). The residue was purified by Flash-Prep-HPLC with the following conditions (IntelFlash-1): Column, C18 silica gel; mobile phase, CH₃CN/H₂O (0.05% NH₄HCO₃)=1/3 increasing to CH₃CN/H₂O (0.05% NH₄HCO₃)=4/1 within 25 min, the eluted product was collected at CH₃CN/H₂O (0.05% NH₄HCO₃)=3/2; Detector, UV 254 nm. This resulted in 6 (16.0 g, 35.0 mmol, 33.3% yield, 90% purity) as a clear oil. ESI-LCMS: m/z 472.2 [M+H₂O]⁻. ¹H NMR (400 MHz, CDCl₃-d) δ 7.75-7.61 (m, 4H), 7.48-7.32 (m, 6H), 6.57-6.43 (m, 1H), 4.83-4.75 (m, 1H), 4.56-4.42 (m, 2H), 4.21-4.10 (m, 2H), 4.05-3.95 (m, 1H), 3.85-3.74 (m, 1H), 1.58 (s, 3H), 1.37 (s, 3H), 1.04 (s, 9H).

Preparation of 7: To a solution of D1M (35.4 g, 133.0 mmol) in dry DCM (100 mL) was added 1 M Et Zn (92.5 mL, 92.5 mmol) at −5° C. The mixture solution was stirred at 0° C. for 15 min. The mixture was added 6 (15.0 g, 33.0 mmol) in dry DCM (50 mL). The mixture solution was stirred at 0° C. for 1 h. The mixture was added D1M (35.4 g, 133.0 mmol), and the mixture solution was stirred at 0˜15° C. for 1.5 h. LC-MS show 6 was completely consumed. The mixture was quenching with NH₄Cl·aq, the product was extracted with DCM. Then washed the organic phase once with saturated brine and dried over by Na₂SO₄. Then the solution was concentrated under reduced pressure. This resulted was purified by silica gel column chromatograph (eluent, PE/EA=10:1˜5:1) twice. This resulted in 6 (9.0 g, 19.2 mmol, 58% yield, 98% purity) as a clear oil. ESI-LCMS: m/z 486.2 [M+H₂O]⁻. ¹H NMR (400 MHz, CDCl₃-d) δ 7.75-7.61 (m, 4H), 7.48-7.32 (m, 6H), 5.86-5.74 (m, 1H), 4.83-4.70 (m, 1H), 4.23-3.91 (m, 3H), 3.82-3.70 (m, 1H), 3.55-3.44 (m, 1H), 1.57 (s, 3H), 1.38 (s, 3H), 1.05 (s, 9H), 0.81-0.71 (m, 1H), 0.66-0.42 (m, 3H).

Preparation of 8: To a solution of 7 (5.0 g, 10.7 mmol) in DCM (75 mL) was added TFA (48.7 g, 427.0 mmol) in H₂O (5.4 mL) at 0° C. The mixture was stirred at 0° C. for 3 h. TLC and LC-MS showed the reaction was complete. The mixture was poured into NaHCO₃·aq, the product was extracted with DCM. Then the combined organic layers were washed the organic phase once with saturated brine and dried over by Na₂SO₄. Then the solution was concentrated under reduced pressure. This resulted in crude 8 (4.7 g) as a clear oil without further purified and used directly for the next step. ESI-LCMS: m/z 446.2 [M+H₂O].

Preparation of 9: To a solution of 8 (4.7 g, 10.6 mmol) in pyridine (40 mL) was added Ac₂O (3.0 g, 2.9 mmol) at r.t. The mixture was stirred at r.t for 16 h. TLC and LC-MS show the reaction was complete. The mixture was poured into NaHCO₃ aq, the product was extracted with DCM. Then washed the organic phase once with saturated brine and dried over by Na₂SO₄. Then the solution was concentrated under reduced pressure. This resulted was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=1/0 within 30 min, the eluted product was collected at CH₃CN/H₂O (0.05% NH₄HCO₃)=7/3; Detector, UV 254 nm. This resulted in 9 (4.4 g, 8.5 mmol, 80% yield over 2 steps, 96% purity) as a clear oil. ESI-LCMS, m/z 530.2 [M+H₂O]⁻. ¹H NMR (400 MHz, CDCl-d) δ 7.75-7.61 (m, 4H), 7.48-7.32 (m, 6H), 6.48-6.09 (m, 1H), 5.46-5.13 (m, 1H), 4.57-4.32 (m, 1H), 4.32-4.03 (m, 1H), 3.97-3.62 (m, 2H), 3.50-3.34 (m, 1H), 2.24-1.85 (m, 6H), 1.13-1.01 (m, 9H), 0.71-0.40 (m, 4H).

### Example 56—Preparation of Compound 351

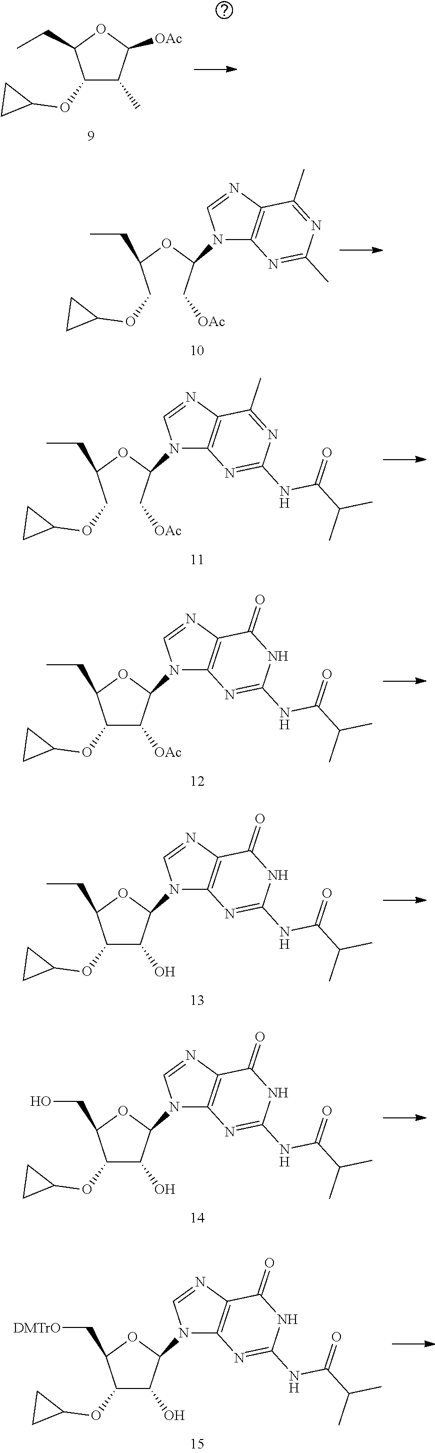

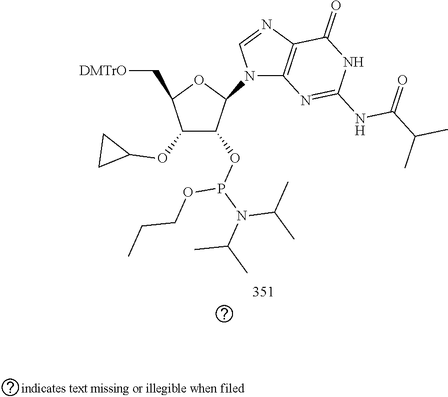

Preparation of 10: To a solution of 9 (5.0 g, 9.8 mmol) in dry ACN (50 mL) was added 6-Chloroguanine (2.5 g, 14.6 mmol) and BSA (6.3 g, 31.3 mmol) at r.t. The mixture solution was stirred at 50° C. for 30 min till the solution was clear. The mixture solution was cooled to 0° C., the mixture was added TMSOTf (2.6 g, 11.9 mmol) drop wise at 0° C. The mixture solution was stirred at 70° C. for 2 h. TLC and LC-MS show 9 was completely consumed. Then the solution was added water (200 mL), the product was extracted with EA, and was washed with water. Then washed the organic phase once with saturated brine and dried over by Na₂SO₄. Then the solution was concentrated under reduced pressure. The residue was isolated by silica gel column chromatography (eluent, PE/EA=2:1˜1:1) to give 10 (5.6 g, 9.0 mmol, 90% yield, 90% purity) as a yellow solid. ESI-LCMS: m/z 622.2 [M+H]⁻. ¹H NMR (400 MHz, DMSO-d₆) δ 8.29 (s, 1H), 7.75-7.54 (m, 4H), 7.50-7.32 (m, 6H), 6.96 (s, 1H), 6.09-6.02 (m, 1H), 5.91-5.82 (m, 1H), 4.69-4.61 (m, 1H), 4.15-4.06 (m, 1H), 3.97-3.69 (m, 2H), 3.56-3.45 (m, 1H), 2.08 (s, 3H), 0.95 (s, 9H), 0.57-0.40 (m, 4H).

Preparation of 11: To a solution of 10 (5.6 g, 9.0 mmol) in pyridine (50 mL) was added iBuCl (1.2 g, 11.7 mmol) at 0° C. The mixture solution was stirred at 25° C. for 3 h. LC-MS show SM was completely consumed. The residue was diluted with EA (200 mL) and was washed with water. Then washed the organic phase once with saturated brine and dried over by Na₂SO₄. Then the solution was concentrated under reduced pressure. This resulted in crude 11 (6.2 g) as a yellow solid. ESI-LCMS: m/z 692.4 [M+H]⁻.

Preparation of 12: To a solution of 11 (6.2 g, 9.0 mmol) in DMF (110 mL) was added TEA (2.8 g, 27.4 mmol), CsOAc (5.1 g, 26.5 mmol) and DABCO (1.0 g, 8.9 mmol) at r.t. The mixture solution was stirred at 25° C. for 3 h. LCMS show SM was completely consumed. The residue was diluted with EA (300 mL) and was washed with water. Then washed the organic phase once with saturated brine and dried over by Na₂SO₄. Then the solution was concentrated under reduced pressure. Then the solution was concentrated under reduced pressure. This resulted in crude 12 (6.8 g) as a yellow solid. ESI-LCMS: m/z 674.2 [M+H]⁻.

Preparation of 13: To a solution of 12 (6.5 g, 9.0 mol) in pyridine (50 mL) was added 2 N NaOH in MeOH/H₂O=4:1 (20 mL) at 0° C. The mixture solution was stirred at 0° C. for 30 min. LC-MS show SM was completely consumed. The mixture was quenching with NH₄Cl·aq, the product was extracted with EA. Then washed the organic phase once with saturated brine and dried over by Na₂SO₄. Then the solution was concentrated under reduced pressure. This resulted was purified twice by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=1/0 within 20 min, the eluted product was collected at CH₃CN/H₂O (0.05% NH₄HCO₃)=3/2; Detector, UV 254 nm. This resulted in 13 (3.1 g, 4.9 mmol, 58% yield over 3 steps, 95% purity) as a white solid. ESI-LCMS: m/z 632.5 [M+H]⁻. ¹H NMR (400 MHz, DMSO-d₆) δ 12.08 (s, 1H), 11.68 (s, 1H), 8.16 (s, 1H), 7.75-7.54 (m, 4H), 7.50-7.32 (m, 6H), 5.87-5.80 (m, 1H), 5.69-5.63 (m, 1H), 4.71-4.61 (m, 1H), 4.20-4.13 (m, 2H), 3.94-3.69 (m, 2H), 3.56-3.56 (m, 1H), 2.83-2.70 (m, 1H), 1.15-1.09 (m, 6H), 1.015 (s, 9H), 0.65-0.40 (m, 4H).

Preparation of 14: To a solution of 13 (3.1 g, 4.9 mmol) in THF (30 mL) was added TBAF (7.3 mL, 7.3 mmol) at 25° C. The mixture solution was stirred at 25° C. for 2 h. LC-MS show SM was completely consumed. Then the solution was concentrated under reduced pressure. The residue was diluted with EA (300 mL) and was washed with water. Then washed the organic phase once with saturated brine and dried over by Na₂SO₄. Then the solution was concentrated under reduced pressure. The residue was purified twice by silica gel column chromatograph (eluent, DCM/MeOH=50:1). This resulted in 27 (1.6 g, 4.0 mmol, 81% yield, 96% purity) as a white solid. ESI-LCMS: m/z 394.0 [M+H]⁻. 1H NMR (400 MHz, DMSO-d₆) δ 12.07 (s, 1H), 11.69 (s, 1H), 8.28 (s, 1H), 5.86-5.77 (m, 1H), 5.58-5.47 (m, 1H), 5.17-5.05 (m, 1H), 4.63-4.49 (m, 1H), 4.11-3.91 (m, 2H), 3.72-3.46 (m, 3H), 2.83-2.70 (m, 1H), 1.19-1.04 (m, 6H), 0.65-0.40 (m, 4H).

Preparation of 15: To a solution of 14 (1.6 g, 4.0 mmol) in pyridine (15 mL) was added DMTrCl (1.7 g, 5.0 mmol) at 25° C. The mixture solution was stirred at 25° C. for 2 h. LC-MS show SM was completely consumed. Then the solution was concentrated under reduced pressure. The residue was diluted with EA (300 mL) and was washed with water. Then washed the organic phase once with saturated brine and dried over by Na₂SO₄. Then the solution was concentrated under reduced pressure. The residue was purified by silica gel column chromatograph (eluent, EA/PE=2:1) to give the crude. The crude was purified by Flash-Prep-HPLC with the following conditions (IntelFlash-1): Column, C18 silica gel; mobile phase, CH₃CN/H₂O (0.05% NH₄HCO₃)=2/3 increasing to CH₃CN/H₂O (0.05% NH₄HCO₃)=1/0 within 20 min, the eluted product was collected at CH₃CN/H₂O (0.05% NH₄HCO₃)=3/2; Detector, UV 254 nm. This resulted in 15 (2.1 g, 3.0 mmol, 75% yield, 99% purity) as a white solid. ESI-LCMS: m/z 696.2 [M+H]⁻. ¹H NMR (400 MHz, DMSO-d₆) δ 12.08 (s, 1H), 11.69 (s, 1H), 8.16 (s, 1H), 7.39-7.17 (m, 9H), 6.92-6.79 (m, 4H), 5.86-5.77 (m, 1H), 5.68-5.57 (m, 1H), 4.78-4.69 (m, 1H), 4.14-4.02 (m, 2H), 3.73 (s, 6H), 3.59-3.50 (m, 1H), 3.31-3.15 (m, 2H), 2.84-2.71 (m, 1H), 1.16-1.07 (m, 6H), 0.65-0.370 (m, 4H).

Preparation of 351: To a solution of 15 (1.9 g, 2.7 mmol) in DCM (20 mL) was added DCI (274 mg, 2.3 mmol) and CEP[N(iPr)₂]₂ (1.1 g, 3.4 mmol) at r.t at N₂. The mixture was stirred at r.t at N₂ for 2 h. LC-MS showed all precursor was consumed completely. The mixture was added NaHCO₃ aqueous (100 mL) and extracted with DCM (100 mL). Then the organic layer was washed with water (200 mL) and brine (200 mL) and dried over Na₂SO₄. The solution was filtered and the filter was concentrated to give the crude. The crude was purified by Flash-Prep-HPLC with the following conditions (IntelFlash-1): Column, C18 silica gel; mobile phase, CH₃CN/H₂O (0.05% NH₄HCO₃)=3/2 increasing to CH₃CN/H₂O (0.05% NH₄HCO₃)=1/0 within 20 min, the eluted product was collected at CH₃CN/H₂O (0.05% NH₄HCO₃)=1/0; Detector, UV 254 nm. This resulted in 351 (1.9 g, 2.1 mmol, 77% yield, 99% purity) as a white solid. ESI-LCMS: m/z 896.2 [M+H]⁻. ¹H NMR (400 MHz, DMSO-d₆) δ 12.14-12.01 (m, 1H), 11.64 (6, 1H), 8.22-8.12 (m, 1H), 7.42-7.15 (m, 9H), 6.92-6.79 (m, 4H), 6.03-5.90 (m, 1H), 5.04-4.88 (m, 1H), 4.24-4.03 (m, 2H), 3.81-3.38 (m, 11H), 3.38-3.16 (m, 2H), 2.84-2.69 (m, 2H), 2.65-2.52 (m, 1H), 1.67-0.95 (m, 15H), 0.87-0.75 (m, 3H), 0.62-0.36 (m, 4H), ³¹P NMR. (600 MHz, DMSO-d₆) δ 150.37, 149.89.

### Example 57—Preparation of Compound 359

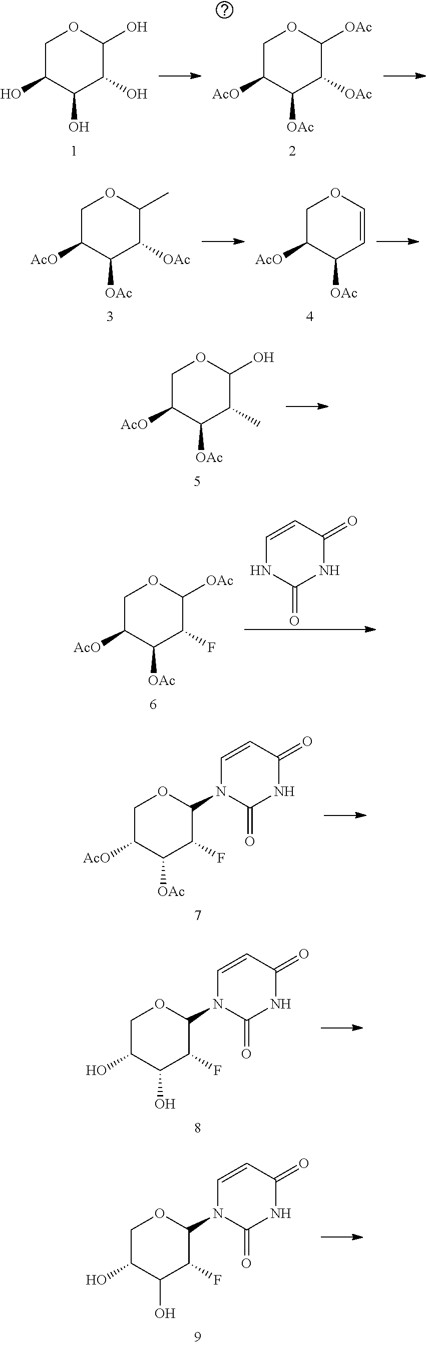

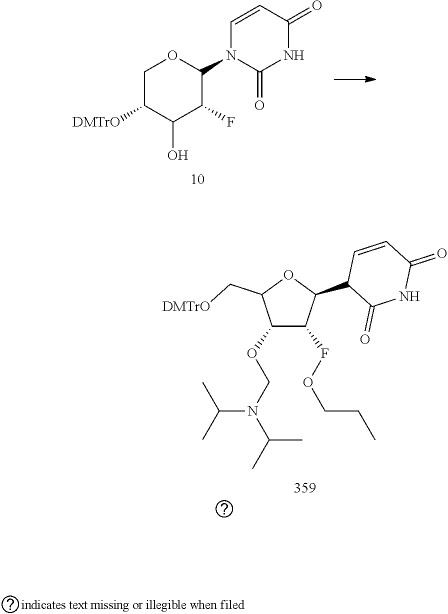

Preparation of 2: To a well stirred suspension of 1 (70.0 g, 470.0 mmol) in dry pyridine (190 mL) was slowly added Ac₂O (271.3 g, 2.6 mol) at 0° C. The suspension was then stirred at room temperature for 4 h, after which it became a light brown colored solution, TLC showed 1 was consumed. The solution was concentrated to give crude 2 (140.0 g, 440.0 mmol, 94.5% yield) as a clear oil, used in the next step directly without any further purification.

Preparation of 3: Crude 2 (140.0 g, 440.0 mmol) was dissolved in DCM (400 mL) and Ac₂O ((6 mL), and a solution of 30% wt HBr in AcOH (280 mL) was added dropwise, the mixture solution was stirred for 12 h at r.t., LTC showed 2 was consumed, then the solution was washed with water (3×500 mL), saturated NaHCO₃ (3×500 mL) and water (3×500 mL), dried over Na₂SO₄, filtered and evaporated to get a syrup that was crystallized from PE to afford 4 (70.0 g, 210.0 mmol, 47% yield), as a white solid. ¹H NMR (400 MHz, CDCl₃) δ 6.71-6.69 (m, 1H), 5.43-5.39 (m, 2H), 5.12-5.06 (m, 1H), 4.24-4.18 (m, 1H), 3.96-3.91 (m, 1H), 2.16 (s, 3H), 2.12 (s, 3H), 2.03 (3, 3H).

Preparation of 4:4-picoline (13.7 g, 147.5 mmol) was added to a suspension of Zn (57.5 g, 884.6 mmol) in EA (500 ml). The mixture was refluxed with vigorous stirring at 80° C., and then a solution of 3 (50.0 g, 147.5 mmol) in EA (150 mL) was added over 30 min. After 60 min, the mixture was cooled down and filtered over a pad of celite. The filtrate was washed with 10% aqueous HCl and saturated NaHCO₃, dried over Na₂SO₄ and concentrated under vacuum. The residue was purified by flash column chromatography using PE/EA=10:1˜5:1 as the eluent to afford 4 (23 g, 115.0 mmol, 79.3% yield) as a white solid. ¹H NMR (400 MHz, CDCl₃) δ 6.52-6.49 (m, 1H), 5.46-5.43 (m, 1H), 5.21-5.16 (m, 1H), 4.87-4.84 (m, 1H) 4.03-3.96 (m, 2H), 2.13-2.04 (m, 6H).

Preparation of 5: To a well stirred solution of 4 (15.0 g, 75.0 mmol) in CH₃NO₂ and water (2:1, 200 mL) was added Selectfluor™ (26.6 g, 90.0 mmol). The solution was stirred overnight at room temperature. The solution was then heated at reflux for 1 h, TLC show 4 was consumed completely. After cooling to room temperature, the CH₃NO₂ was removed in vacuo. Water was added and extracted with EA. The combined organic fractions were successively washed with 1N HCl (100 mL) and water, dried over Na₂SO₄, filtered and evaporated to afford 5 (11.2 g, 47.4 mmol, 63% yield). ¹H NMR (400 MHz, CDCl₃) δ 5.23-5.06 (m, 1H), 4.95-4.80 (m, 1H), 4.76-4.72 (m, 1H), 4.10-3.94 (m, 1H), 3.79-3.65 (m, 3H), 2.19-2.02 (m, 6H).

Preparation of 6: To a well stirred suspension of 5 (11.0 g, 46.6 mmol) in dry pyridine (35 ml) at 0° C., was slowly added acetyl acetate (9.5 g, 93.2 mmol). The suspension was then stirred at room temperature for 4 h, after which it became a light brown colored solution TLC showed 5 was consumed, the pyridine was removed in vacuo. The reaction mixture was diluted with EA (400 mL), and successively washed with water (3×300 mL), saturated NaHCO₃ (3×300 mL) and NaCl (3×300 mL), dried over Na₂SO₄, filtered and concentrated under vacuum. The residue was purified by flash column chromatography using PE/EA=5:1 as the eluent to afford 6 (11.0 g, 39.6 mmol, 84.6% yield) as a light yellow oil. ¹H NMR (400 MHz, CDCl₃) δ 6.44-6.43 (m, 0.5H), 5.74-5.71 (m, 0.5H), 5.44-5.39 (m, 1H), 5.34-5.33 (m, 1H), 5.20-5.17 (m, 1H), 4.99-4.97 (m, 1H), 4.88-4.82 (m, 1H), 4.76-3.71 (m, 1H), 4.64-4.58 (m, 1H), 4.05-4.02 (m, 1H), 4.83-4.77 (m, 1H), 2.19-2.02 (m, 9H); ¹⁹F NMR (400 MHz, CDCl₃) δ 205.10, 207.26.

Preparation of 7: To a well stirred suspension of 6 (10.0 g, 36.0 mmol) and uracil (4.0 g, 36.0 mmol) in CH₃CN (100 ml). BSA (14.6 g, 71.8 mmol) was added into the reaction mixture solution and heated to 70° C. for 45 min under N₂. Then the TMSOTf (24.0 g, 108.0 mmol) was added into the reaction mixture. TLC showed 6 was consumed completely. The reaction mixture was diluted with EA (500 mL), and successively washed with saturated NaHCO₃ (3×1000 mL), dried over Na₂SO₄, filtered and concentrated under vacuum. The residue was purified by flash column chromatography using PE/EA=5:1 as the eluent to afford 7 (9.0 g, 27.2 mmol, 76% yield) as a white solid. ESI-LCMS: m/z 331.3 [M+H]⁺; ¹H NMR (400 MHz, DMSO-d₆) δ 11.57 (s, 1H), 7.8-7.76 (m, 1H), 6.00-5.95 (m, 1H), 5.76-5.71 (m, 1H), 5.55-5.47 (m, 1H), 5.30-5.04 (m, 2H), 4.15-4.09 (m, 1H), 3.96-3.91 (m, 1H), 2.14 (s, 3H), 2.02 (s, 3H).

Preparation of 8: To a solution of 7 (9.0 g, 27.2 mmol) in NH₃-MeOH (200 mL), then the reaction mixture was stirred at r.t. for 5 h. LCMS and TLC showed that the raw material was disappeared, the MeOH was removed in vacuo. The reaction mixture was diluted with DCM (400 mL), and saturated NaHCO₃ (3×500 mL) and NaCl (3×500 mL), dried over Na₂SO₄, filtered and concentrated under vacuum. The residue was purified by flash column chromatography using DCM/MeOH=15:1˜10:1 as the eluent to afford 8 (4.5 g, 18.3 mmol, 67.2% yield) as a white solid. LCMS: m/z=247.3 [M+H]⁺; ¹H NMR (400 MHz, DMSO-d₆) δ 11.47 (s, 1H), 7.74-7.71 (m, 1H), 5.73-5.71 (d, J=8.0, 1H), 5.63-5.59 (m, 1H), 5.48-5.46 (d, J=8.0, 1H), 5.02-5.01 (d, J=4.0, 1H), 4.77-4.58 (m, 1H), 3.92-3.3.81 (m, 1H), 3.78-3.71 (m, 3H).

Preparation of 9: To a solution of 8 (4.5 g, 18.3 mmol) in a mixture solvent of dioxane (67 ml) and H₂O (22 ml) at r.t., NaIO₄ (5.9 g, 36.6 mmol) was added into the mixture reaction, 9 was consumed by TLC and LCMS, the mixture was cooled down to turn 0° C., NaBH₄ (765.0 mg, 20.1 mmol) was added into the mixture reaction, the reaction mixture was allowed to stir for 2 h at 0° C., the reaction was adjusted pH=7 by the aqueous 2N HCl, then the reaction was concentrated to get crude which was purified by silica gel (DCM:MeOH=20:1˜10:1) to give compound 9 (2.2 g, 8.9 mmol, 50% yield) as a white solid. LCMS: m/z=271.3 [M+Na]⁺; ¹HNMR (400 MHz, DMSO-d₆) δ 11.41 (s, 1H), 7.73-7.66 (m, 1H), 5.93-5.82 (m, 1H), 5.74-5.64 (m, 1H), 5.22-5.14 (m, 1H), 4.80-4.6 (m, 2H), 3.84-3.41 (m, 6H).

Preparation of 10: To a solution of 9 (2.2 g, 8.9 mmol) in pyridine (22 mL) was added DMTrCl (3.0 g, 8.9 mmol), then the reaction mixture was stirred at rt. for 2 h, LCMS shows that the raw material was consumed. Then the reaction solution was poured into ice-water, and extracted with EA, wished by brine, dried over Na₂SO₄, filtered and concentrated to get residue which was purified by silica gel column to give compound 10 (3.0 g, 5.5 mmol, 61% yield). LCMS: m/z=549.3 [M−H]⁺; ¹H NMR (400 MHz, DMSO-d₆) δ 11.47 (s, 1H), 7.53-7.32 (d, J=8.4, 1H), 7.38-7.22 (m, 9H), 6.89-6.87 (m, 4H), 5.90-5.88 (m, 1H), 5.70-5.68 (m, 1H), 5.20-5.17 (m, 1H), 4.85-4.69 (m, 1H), 3.74-3.3.56 (m, 10H), 3.27-2.95 (m, 2H); LCMS: m/z=247.3 [M+H]⁺.

Preparation of 359: To a suspension of 10 (3 g, 5.5 mmol) in DCM (30 mL) was added DCI (0.58 g, 4.9 mmol) and CEP[N(iPr)₂]₂ (2.14 g, 7.1 mmol). The mixture was stirred at r.t. for 1 h. LC-MS showed 10 was consumed completely. The solution was washed with a solution of NaHCO₃twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=1/0; Detector, UV 254 nm. This resulted in 359 (2.7 g, 3.6 mmol, 66% yield) as a white solid. ESI-LCMS: m/z 749.3 [M−H]⁺; ¹H NMR (400 MHz, DMSO-d₆) δ 11.43 (s, 1H), 7.76-7.70 (m, 1H), 7.39-7.35 (m, 2H), 7.33-7.27 (m, 2H), 7.26-7.21 (m, 5H), 6.91-6.85 (m, 4H), 5.73-5.67 (m, 1H), 5.07-4.89 (m, 1H), 4.03-3.89 (m, 1H), 3.84-3.51 (m, 14H), 3.19-2.97 (m, 2H), 2.79-2.83 (m, 2H), 1.16-1.01 (m, 12H); ³¹P NMR (162 MHz, DMSO-d₆) δ 148.98, 148.94, 148.89.

### Additional Tables

TABLE 41

| SEQ ID NO: | Description | Sequence⁺ |
|---|---|---|
| 89 | Hepatitis B | aattccacaaccttteaccaaactctgcaagateccagagtgagaggcetgtatttccctg |
| | virus | ctggtggctccagttcaggagcagtaaaccctgttcegactactgcctetcccttategtca |
| | (Genbank | atcttctcgaggattggggaccctgcgctgaacatggagaacatcacatcaggattecta |
| | Accession | ggaccccttctcgtgttacaggcggggtttttcttgttgacaagaatcctcacaataccgca |
| | No. | gagtctagactcgtggtggacttctctcaattttctagggggaactaccgtgtgtcttggcc |
| | U95551.1) | aaaattcgcagtccccaacctccaatcactcaccaacctcctgtcctccaacttgtcctggt |
| | | tatcgctggatgtgtctgeggegttttatcatcttcctetteatcctgctgctatgectcatettc |
| | | ttgttggttcttctggactatcaaggtatgttgcccgtttgtcctctaattccaggatcctcaac |
| | | caccagcacgggaccatgccgaacctgcatgactactgctcaaggaacctctatgtatec |
| | | ctcctgttgctgtaccaaaccttcggacggaaattgcacctgtattcccateccatcatcct |
| | | gggcttteggaaaattectatgggagtgggectcagccegtttctectggctcagtttacta |
| | | gtgccatttgttcagtggttogtagggctttcccccactgtttggctttcagttatatggatgat |
| | | gtggtattgggggccaagtctgtacagcatettgagtccctttttaccgetgttaccaattttc |
| | | ttttgtctttgggtatacatttaaaccctaacaaaacaaagagatggggttactctctgaatttt |
| | | atgggttatgtcattggaagttatgggtccttgccacaagaacacatcatacaaaaaatca |
| | | aagaatgttttagaaaacttcctattaacaggcctattgattggaaagtatgtcaacgaattg |
| | | tgggtcttttgggttttgctgccccatttacacaatgtggttatcctgegttaatgcccttgtat |
| | | gcatgtattcaatctaagcaggctttcactttctogccaacttacaaggcctttctgtgtaaac |
| | | aatacctgaacctttaccccgttgcccggcaacggccaggtctgtgccaagtgtttgctga |
| | | cgcaacccccactggctggggcttggtcatgggccatcagegcgtgcgtggaacctttt |
| | | eggctcctctgccgatccatactgeggaactectagecgcttgttttgctcgcagcaggtc |
| | | tggagcaaacattatcgggactgataactetgttgtcetetcccgcaaatatacategtatc |
| | | catggctgctaggctgtgctgccaactggatcctgcgcgggacgtcctttgtttacgtccc |
| | | gtcggcgctgaatcctgeggacgaccettcteggggtegettgggactetetegtcccctt |
| | | ctccgtetgcegttecgaccgaccacggggegcacctetetttacgeggactccccgtct |
| | | gtgccttetcatctgceggaccgtgtgcacttegettcacetetgcacgtcgcatggagac |
| | | caccgtgaacgcccaccgaatgttgcccaaggtcttacataagaggactcttggactetct |
| | | gcaatgtcaacgaccgaccttgaggcatacttcaaagactgtttgtttaaagactgggagg |
| | | agttgggggaggagattagattaaaggtctttgtactaggaggctgtaggcataaattggt |
| | | ctgcgcaccagcaccatgcaactttttcacctetgcctaatcatetettgttcatgtcctactg |
| | | ttcaagcctccaagctgtgccttgggtggctttggggcatggacategaccettataaaga |
| | | atttggagctactgtggagttactctegtttttgccttetgacttetttccttcagtacgagatct |
| | | tetagataccgcctcagetctgtategggaagccttagagtetcctgagcattgttcacctc |
| | | accatactgcactcaggcaagcaattctttgetggggggaactaatgactctagctacctg |
| | | ggtgggtgttaatttggaagatccagcatctagagacctagtagtcagttatgtcaacacta |
| | | atatgggcctaaagttcaggcaactcttgtggtttcacatttcttgtctcacttttggaagaga |
| | | aaccgttatagagtatttggtgtcttteggagtgtggattegcactcetccagettatagacc |
| | | accaaatgcccctatcctatcaacacttccggaaactactgttgttagacgacgaggcag |
| | | gtcccctagaagaagaactecctogcctegcagacgaaggtctcaategccgegtegca |
| | | gaagatctcaatctegggaacctcaatgttagtattccttggactcataaggtggggaactt |
| | | tactggtctttattettctactgtacctgtctttaatectcattggaaaacaccatettttcctaat |
| | | atacatttacaccaagacattatcaaaaaatgtgaacagtttgtaggcccacttacagttaat |
| | | gagaaaagaagattgcaattgattatgcctgctaggttttatccaaaggttaccaaatattta |
| | | ccattggataagggtattaaaccttattatccagaacatctagttaatcattacttccaaacta |
| | | gacactatttacacactctatggaaggegggtatattatataagagagaaacaacacatag |
| | | cgcctcattttgtgggtcaccatattcttgggaacaagatctacagcatggggcagaatett |
| | | tccaccagcaatcctctgggattctttcccgaccaccagttggatccagccttcagagcaa |
| | | acacagcaaatccagattgggacttcaatcccaacaaggacacctggccagacgccaa |
| | | caaggtaggagctggagcattegggctgggtttcaccccaccgcacggaggccttttgg |
| | | ggtggagccctcaggctcagggcatactacaaactttgccagcaaatccgcctectgcct |
| | | ccaccaategccagacaggaaggcagcctaccccgetgtetccacctttgagaaacact |
| | | catcctcaggccatgcagtgg |
| 90 | MCJ mRNA | agtcactgccgeggegccttgagtctccgggccgccttgccatggctgcccgtggtgtcatc |
| | (GenBank | gctccagttggcgagagtttgcgctacgetgagtacttgcagccctcggccaaacggccag |
| | Accession | acgccgacgtcgaccagcagagactggtaagaagtttgatagctgtaggactgggtgttgc |
| | No. | agctcttgcatttgcaggtcgctacgcatttcggatctggaaacctctagaacaagttatcaca |
| | NM_01323 | gaaactgcaaagaagatttcaactcctagcttttcatcctactataaaggaggatttgaacaga |
| | 8.3) | aaatgagtaggcgagaagctggtcttattttaggtglaagcccatctgctggcaaggctaaga |
| | | ttagaacagctcataggagagtcatgattttgaatcacccagataaaggtggatctccttacgt |
| | | agcagccaaaataaatgaagcaaaagacttgctagaaacaaccaccaaacattgatgettaa |
| | | ggaccacactgaaggaaaaaaaaagaggggacttegaaaaaaaaaaaagccctgcaaaa |
| | | tattctaaaacatggtcttcttaattttctatatggattgaccacagtcttatcttccaccattaagct |
| | | gtataacaataaaatgttaatagtcttgctttttattatottttaaagatctccttaaattctataactg |
| | | atcttttttcttattttgtttgtgacattcatacatttttaagatttttgttatgttetgaattcccccctac |
| | | acacacacacacacacacacacacacacacgtgcaaaaaatatgatcaagaatgcaattgg |
| | | gatttgtgagcaatgagtagacctcttattgtttatatttgtaccctcattgtcaatttttttttaggg |
| | | aatttgggactctgcctatataaggtgttttaaatgtettgagaacaagcactggctgatacctc |
| | | ttggagatatgatctgaaatgtaatggaatttattaaatggtgtttagtaaagtaggggttaagg |
| | | acttgttaaagaaccccactatctctgagaccctatagccaaagcatgaggacttggagagct |
| | | actaaaatgattcaggtttacaaaatgagccctgtgaggaaaggttgagagaagtctgagga |
| | | gtttgtatttaattatagtcttccagtactgtatattcattcattactcattctacaaatatttattgacc |
| | | ccttttgatgtgcaaggcactategtgcgtcccctgagagttgcaagtatgaagcagtcatgg |
| | | atcatgaaccaaaggaacttatatgtagaggaaggataaatcacaaatagtgaatactgttag |
| | | atacagatgatatattttaaaagttcaaaggaagaaaagaatgtgttaaacactgcatgagag |
| | | gaggaataagtggcatagagctaggctttagaaaagaaaaatattccgataccatatgattgg |
| | | tgaggtaagtgttattctgagatgagaattagcagaaatagatatatcaatcggagtgattaga |
| | | gtgcagggtttctggaaagcaaggtttggacagagtggtcatcaaaggccagccctgtgact |
| | | tacactgcattaaattaatttcttagaacatagtccctgatcattatcactttactattccaaaggt |
| | | gagagaacagattcagatagagtgccagcattgttteccagtattectttacaaatcttgggttc |
| | | attccaggtaaactgaactactgcattgtttctatottaaaatactttttagatatcctagatgcatc |
| | | tttcaacttctaacattctgtagtttaggagttctcaaccttggcattattgacatgttaggccaaa |
| | | taattttttttgtgggaggtctcttgtgegttttagatgattagcaataatccctgacctgttatcta |
| | | ctaaagactagtcgtttctcatcagttgtgacaacaaaaatggttccagatattgccaaatgcc |
| | | ctttagaggacagtaatcgcccccagttgagaaccatttcagtaaaactttaattactattttttct |
| | | tttggtttataaaataatgatcctgaattaaattgatggaaccttgaagtegataaaatatatttett |
| | | getttaaagtccccatacgtgtectactaattttctcatgetttagtgttttcacttttetcctgttatc |
| | | cttgtacctaagaatgccatcccaatccccagatgtccacctgcccaaagtctaggcatagct |
| | | gaaggccaagctaaaatgtatccctctttttctggtacatgcagcaaaagtaatatgaattatca |
| | | gctttctgagagcaggcattgtatctgtettgtttggtgttacattggcacccaataaatatttgtt |
| | | gagtgaatgaataaattcccatagcactttattcttcacatggtacataactataggggctatag |
| | | cttggtaccttgtgaagcaactcttggtgtaacataccttatttctcatactaaaatgcaagaacc |
| | | tttagagcaaggatcttgccattcatctttgtaacctctttactctggagcacttgcatttagcag |
| | | gcatcataaagttttacgtaccaagaaaatgttgctgttttctgaatactatgcatcaaaaaatgt |
| | | taccactaatttttaaagetctgctaaggaatattggggcaccctcagatgcaccttttaattgat |
| | | gtcatattttcctaatccatactttattcatgagaatttgagtcaccccagcattagcttggaatttc |
| | | cttatttcccatttgctttgcaggtgccttggagtcagatctggttttgaatactatettectgttat |
| | | gtgatcttgggcagttacttaattttctagtcaataacccgtatctataaaatagagaaaataatc |
| | | ctacacaccggggcctgttgtggggggggagaggggggagggatcgcatttggagatat |
| | | actaatgtaaatgacaagttaattggtgcagcacaccaacatggctcatgtctacatatgtaac |
| | | aaacctgcacgttgtgcacatgtgccctagaacttaaagtataataaaaagaaattttaaaaaa |
| | | tcctgtcaaataaggttatagtagagaataaggatgtgtaaagcatttagtcacgtaaatgotta |
| | | aaaaaatgtaatttttacttotttcactgcctcatttaattagttttatotttaataataccttggattca |
| | | gggtaaagtttcagttatgtcccagtaatcatttattttaccctcgaatctgcaatttggatagaa |
| | | catggtggggacagctegtctctattccttgcagcattaacaggetggaggcaccacttetct |
| | | ggccagcaagttgggcctggttgttggctgagagcctcagttcctttctgcacaggttcctctt |
| | | tacataggcttctcaacagggctactagagcategtcaccatagcagctgtcttataacagag |
| | | agtggtcggtctgagagacaaaaaatggaagctgccaaattgttctgggtctggaaactgtc |
| | | agggcatcacttgtgccatattcagttggcctaagaattacagagcctgcctcgattcaaagg |
| | | gagaggatagagaggactgaaggaatcagtgctcatctttaatatgcagcaggacaggtttg |
| | | ggattttttttcccccttgagtctgtgaaggcattacttaagaacaaagtcaggcatgtataattg |
| | | aactacagttacttgaaatataagcccagaaagtttcagataataaatacaactatttttctgctg |
| | | ttacccttgtacctaaagatgccatcctaatccccagatctccacaactatacctacatagtaga |
| | | aggttaaaatgtatccctctttttctggtgcatccagcaaaagtaatatcatgaattatgagctct |
| | | ctgagagcaaggatcatatcagtcttgtttattgttgcagtgaacaagtacagttgcagatattc |
| | | aggagtaattatctaaatggcagtaggettataaaactgaattttcaccagccacaccctcccc |
| | | ccaactccttatctgtaaaaagcttatttgagtggttacctgtcttcagtaaagattgcgcttgca |
| | | tatttgctgtcattgcatattctgcttaattaagctctgttgatattgcagtttctgtgcatacttacat |
| | | cttagatgcaatctgagggcctaggaaggccttttaaaaataaaacaattccgattgcagaga |
| | | aagtgtaagtcaaggacagttaattcaaggggaacatagaaagctatttagattttagttgatg |
| | | gtgccagtcttcagegtaaagtcaaaagtggagggaagtttagtaaggaaaaaatgttgggc |
| | | ttggaatacattgtttagtcttcaaagcactttactttttatgaaatatattttagacattcagcaaat |
| | | attgaatacttactatatcaggcagtaaagatataaattcattcttaaaatgtgcaacatgttcaa |
| | | actgaaaaaaatacattcttaaacaggaaactttttccttcatactttttaattaacaagacatata |
| | | agagttgcattaatgggcgtgcttatgattgatcacccagcagcatcattagaaataatatatttt |
| | | attcatgtgcagaaatcttttggttgtcctggggaaccttgaacacagaaaagagcttttattga |
| | | taaggtaattgaacacacttgacaattagcttaatatggtttaataccatttgtgggagaagatg |
| | | aatcagccaggctctttacgtcaagaatatgaagtttctettgagtcaaccaacttaagatgag |
| | | ctacggagactgcagtgaaaagttaaatatccaagtacaccagccaatttcacacagtggaa |
| | | ccatgtgtcetegggcaccctgcacctegoccaacagtcatcaactagatggaggctoctg |
| | | gctgcaaggaggatttgatgggaatgagtaaatgtgtcagcatagtccgtcccttctaatgga |
| | | aaagcaacccaaagagcaaatcctattaatggetggatcagtatcatctacttgtcaaaaaca |
| | | ttccatgaattatgagtcaaaattttatttatggtggcattacacacattaagagatgaggacttc |
| | | tgttagcataatttattagctggaaaagttgagaaggttctctggactcatttttataggtggaac |
| | | ctaagtgatctggataattgcccaccagcaaaattgctgggcatggtggacaaagaaaatgtt |
| | | ccttctaatgattttttatgagctgagtagctattgttcccagctgagtgctcttttcctctttttattg |
| | | ttgctgagcaaaagaatttataaaaagctctttcttttgtattaaaaaccctgctcaattgaaatg |
| | | caagttcattaagtaatetteatttetettcetgecataataaccetttcectetetgttcgattcaa |
| | | cagtatctagcagcactgctccaaattttaagtctgaacagactatattacatagatgtagaga |
| | | aatactcaatcttcagcattaagagggagcttaatttcacacgggtggaatatgatcactcagg |
| | | ctagatgttggccataaatttcaaattagtatctcaacttagcaggggggatcaacagtggca |
| | | aacttcaattatgacaggataaaaatcacatagagatattggttcaatatggacatctaaactat |
| | | aatgctaaaagccaataattagaataagttcattttaagaaaagcattaataatattagctaacg |
| | | tttagtacctgtgccaaacattctacctatgttaccttgattttcatagccagcctaagaggtact |
| | | attatgtatccccattttacaggttaagaaacaggctcagaggagtttaggatottttccaagatt |
| | | acatagccagtaagtggtggcactaggaaccaaattcagactctgaatcgcatgctgtttatat |
| | | tatattgcactcattctaaatatgtgggaatcagaatgaaggggcttgtatgacttttggctcatt |
| | | ttttgatgcatgtgacctgggattataaatgtgaaattaggtttacgaaaggatccagtgtcatt |
| | | gtgcatcatgggcaaggagtacctaatctctttaattettccctggaagcttacgatgtccatcc |
| | | aagtgcacatagcaaaagttctgttgtaaagtttagcagagtgactttetttgactcagagtgat |
| | | gacggaggaagctttgataagattttatctgaaatgttcatggacaagagctttcaaggagaa |
| | | catccagagcaaggttctgaagacagctcatgaaggtgaagcagcagacctggcacaaga |
| | | aatgaagagagagctcagtgtattaaagatgaaaacaagaaaaccgaatatattgaaagga |
| | | gcagagaggcaatgaaaacaagacaactgaaatgaggtaacttgcagcaattgaaaggga |
| | | atttcagtacttttatagaattcttaaaaattgtttcctgctgtttattttcaattttgaacagggttatt |
| | | tgtccatgccatactttttttgccaaattccaaaattgtgtatagttctatagttgtctggtggagtc |
| | | aatggaactttagttaccagtctaagaatgtgtctttgagattgtccagttaattctctatttccagt |
| | | agctgtaataaatggtgaaaaggtttctgactcctggagaaagtttctaactccttatgactaat |
| | | attcataacagacttgtgagttccttgaacatggatacacctatatgcaagagtgtattccaaag |
| | | ctaactcagtgatctttccatttatctattcttggattagtggtgcctttgctotttccttctgtaaatg |
| | | tgaatagttaagagttgactgcagaagtgtttacactttggcttccatgcctctggaatgtttgtg |
| | | ctttggtggtgagatgtgagactatatttgtatagtctgcatctctcaggctgccccagaatgtt |
| | | gtacagtgcagtgctgaagaaagcagcaggtacacacagaaatgcagcctttcctggttaa |
| | | ccctgcttggatctgagttacactttgtttcctgacttettgggacttaggtaatcagtttgccttct |
| | | actctatctcattttgtactcgcttacatactacattcttgtttgggctttcgtttcttcttgtaagcag |
| | | agattttttaaaatccaatatgtgaaaatacggatgcactacaattaaataaataaaatgctgttg |
| | | tgtttgttttgctttaaaattgtaaaggataaacaataagatagttttatctatgtggttttcccgatg |
| | | cagttaaaataaaacctaatctgctaaaattgaa |
| 91 | TAZ | gctttceggcggttgcacegggceggggtgccagegcccgcettccegtttcetcccgttcc |
| | (GenBank | gcagcgcgcccacggcctgtgaccccggcgaccgctccccagtgacgagagagcgggg |
| | Accession | ccgggegetgctceggcctgacctgegaagggaccteggtccagtcecetgttgegcegc |
| | No. | gcccccgtccgtccgtgcgcgggccagtcaggggccagtgtctcgageggtcgaggtcg |
| | NM_00011 | cagacctagaggegccccacaggceggcccggggcgctgggagegccggccgegggc |
| | 6.5) | cgggtggggatgcctctgcacgtgaagtggccgttccccgeggtgccgccgctcacctgg |
| | | accctggccagcagcgtcgtcatgggcttggtgggcacctacagctgcttctggaccaagt |
| | | acatgaaccacctgaccgtgcacaacagggaggtgctgtacgagctcatcgagaagcgag |
| | | gcceggccacgcccctcatcacegtgtccaatcaccagtcctgcatggacgaccetcatetc |
| | | tgggggatcctgaaactccgccacatctggaacctgaagttgatgcgttggacccctgcagc |
| | | tgcagacatctgcttcaccaaggagctacactcccacttcttcagcttgggcaagtgtgtgcct |
| | | gtgtgccgaggagcagaatttttccaagcagagaatgaggggaaaggtgttctagacacag |
| | | gcaggcacatgccaggtgctggaaaaagaagagagaaaggagatggcgtctaccagaag |
| | | gggatggacttcattttggagaagctcaaccatggggactgggtgcatatcttcccagaagg |
| | | gaaagtgaacatgagttccgaattcctgcgtttcaagtggggaatcgggcgcctgattgctg |
| | | agtgtcatctcaaccccatcatcctgcccctgtggcatgtoggaatgaatgacgtcettectaa |
| | | cagtccgccctacttcccccgetttggacagaaaatcactgtgctgategggaagcccttca |
| | | gtgccctgcctgtactcgagcggctccgggcggagaacaagtcggctgtggagatgcgga |
| | | aagccctgacggacttcattcaagaggaattccagcatctgaagactcaggcagagcagct |
| | | ccacaaccacctccagcctgggagataggccttgcttgctgccttctggattcttggcccgca |
| | | cagagctggggctgagggatggactgatgcttttagctcaaacgtggcttttagacagatttg |
| | | ttcatagacectctcaagtgccctetcegagetggtaggcattccagetcetcegtgettectc |
| | | agttacacaaaggacctcagctgcttctcccacttggccaagcagggaggaagaagcttag |
| | | gcagggctctctttccttettgccttcagatgttctctcccaggggctggettcaggagggagc |
| | | atagaaggcaggtgagcaaccagttggctaggggagcagggggcccaccagagctgtg |
| | | gagaggggaccctaagactecteggcctggetcctacccacegcccttgcegaaccagga |
| | | getgctcactacctectcagggatggccgttggccacgtcttcettetgcetgagettcccccc |
| | | caccacaggccctttcctcaggcaaggtctggcctcaggtgggccgcaggcgggaaaag |
| | | cagcccttggccagaagtcaagcccagccacgtggagcctagagtgagggectgaggtct |
| | | ggetgettgcccccatgetggegecaacaacttetecatcctttetgcctetcaacatcacttg |
| | | aatcctagggcctgggttttcatgtttttgaaacagaaccataaagcatatgtgttggcttgttgt |
| | | aaaa |
| 92 | ANGPTL3 | agaagaaaacagttccacgttgettgaaattgaaaatcaagataaaaatgttcacaattaaget |
| | (GenBank | ccttctttttattgttcctctagttatttcctccagaattgatcaagacaattcatcatttgattetctat |
| | Accession | ctccagagccaaaatcaagatttgctatgttagacgatgtaaaaattttagccaatggectcctt |
| | No. | cagttgggacatggtcttaaagactttgtccataagacgaagggccaaattaatgacatatttc |
| | NM_01449 | aaaaactcaacatatttgatcagtctttttatgatctatcgctgcaaaccagtgaaatcaaagaa |
| | 5.4) | gaagaaaaggaactgagaagaactacatataaactacaagtcaaaaatgaagaggtaaag |
| | | aatatgtcacttgaactcaactcaaaacttgaaagcctcctagaagaaaaaattctacttcaac |
| | | aaaaagtgaaatatttagaagagcaactaactaacttaattcaaaatcaacctgaaactccag |
| | | aacacccagaagtaacttcacttaaaacttttgtagaaaaacaagataatagcatcaaagacc |
| | | ttctccagacegtggaagaccaatataaacaattaaaccaacagcatagtcaaataaaagaa |
| | | atagaaaatcagetcagaaggactagtattcaagaacccacagaaatttetctatcttccaag |
| | | ccaagagcaccaagaactactccctttcttcagttgaatgaaataagaaatgtaaaacatgat |
| | | ggcattcctgctgaatgtaccaccatttataacagaggtgaacatacaagtggcatgtatgcc |
| | | atcagacccagcaactctcaagtttttcatgtctactgtgatgttatatcaggtagtccatggac |
| | | attaattcaacategaatagatggatcacaaaacttcaatgaaacgtgggagaactacaaata |
| | | tggttttgggaggcttgatggagaattttggttgggcctagagaagatatactccatagtgaag |
| | | caatctaattatgttttacgaattgagttggaagactggaaagacaacaaacattatattgaata |
| | | ttctttttacttgggaaatcacgaaaccaactatacgctacatctagttgcgattactggcaatgt |
| | | ccccaatgcaatcccggaaaacaaagatttggtgttttctacttgggatcacaaagcaaaagg |
| | | acacttcaactgtccagagggttattcaggaggctggtggtggcatgatgagtgtggagaaa |
| | | acaacctaaatggtaaatataacaaaccaagagcaaaatctaagccagagaggagaagag |
| | | gattatottggaagtctcaaaatggaaggttatactctataaaatcaaccaaaatgttgatccat |
| | | ccaacagattcagaaagctttgaatgaactgaggcaaatttaaaaggcaataatttaaacatta |
| | | acctcattccaagttaatgtggtctaataatctggtattaaatccttaagagaaagcttgagaaa |
| | | tagattttttttatcttaaagtcactgtctatttaagattaaacatacaatcacataaccttaaagaa |
| | | taccgtttacatttctcaatcaaaattcttataatactatttgttttaaattttgtgatgtgggaatcaa |
| | | ttttagatggtcacaatctagattataatcaataggtgaacttattaaataacttttctaaataaaa |
| | | aatttagagacttttattttaaaaggcatcatatgagctaatatcacaacttteccagtttaaaaaa |
| | | ctagtactcttgttaaaactctaaacttgactaaatacagaggactggtaattgtacagttcttaa |
| | | atgttgtagtattaatttcaaaactaaaaategtcagcacagagtatgtgtaaaaatctgtaatac |
| | | aaatttttaaactgatgcttcattttgctacaaaataatttggagtaaatgtttgatatgatttatttat |
| | | gaaacctaatgaagcagaattaaatactgtattaaaataagttcgctgtctttaaacaaatgga |
| | | gatgactactaagtcacattgactttaacatgaggtatcactataccttatttgttaaaatatatac |
| | | tgtatacattttatatattttaacacttaatactatgaaaacaaataattgtaaaggaatcttgtcag |
| | | attacagtaagaatgaacatatttgtggcatcgagttaaagtttatatttcccctaaatatgctgt |
| | | gattctaatacattegtgtaggttttcaagtagaaataaacctegtaacaagttactgaacgttta |
| | | aacagcctgacaagcatgtatatatgtttaaaattcaataaacaaagacccagtccctaaatta |
| | | tagaaatttaaattattcttgcatgtttategacatcacaacagatccctaaatccctaaatcccta |
| | | aagattagatacaaattttttaccacagtatcacttgtcagaatttatttttaaatatgattttttaaa |
| | | actgccagtaagaaattttaaattaaacccatttgttaaaggatatagtgcccaagttatatggt |
| | | gacctacctttgtcaatacttagcattatgtatttcaaattatccaatatacatgtcatatatattttta |
| | | tatgtcacatatataaaagatatgtatgatctatgtgaatcctaagtaaatattttgttccagaaaa |
| | | gtacaaaataataaaggtaaaaataatctataattttcaggaccacagactaagctgtcgaaat |
| | | taacgctgatttttttagggccagaataccaaaatggctcctctettcccccaaaattggacaat |
| | | ttcaaatgcaaaataattcattatttaatatatgagttgcttcctctatttggtttcc |
| 93 | DGAT2 | tgccccgttgtgaggtgataaagtgttgegctcegggacgccagegccgeggetgccgect |
| | (GenBank | clgctggggtctaggetgtttctctegcgccaccactggcegccggccgcagctccaggtgt |
| | Accession | cctagecgcccagcctegacgcegtecegggacecetgtgctetgegegaagcectggcc |
| | No. | ccgggggccggggcatgggccaggggcgcggggtgaageggcttcccgeggggccgt |
| | NM_00125 | gactggggggettcagccatgaagaccctcatagccgcctactccggggtcctgcgcgg |
| | 3891.1) | cgagcgtcaggccgaggctgaccggagccagcgctctcacggaggacctgcgctgtcgc |
| | | gcgaggggtctgggagatggggagtggcctgcagtgccatcctcatgtacatattctgcact |
| | | gattgctggctcatcgctgtgctctacttcacttggctggtgtttgactggaacacacccaaga |
| | | aaggtggcaggaggtcacagtgggtccgaaactgggctgtgtggcgctactttcgagacta |
| | | ctttcccatccagctggtgaagacacacaacctgctgaccaccaggaactatatctttggata |
| | | ccacccccatggtatcatgggcctgggtgccttctgcaacttcagcacagaggccacagaa |
| | | gtgagcaagaagttcccaggcatacggccttacctggctacactggcaggcaacttccgaa |
| | | tgcctgtgttgagggagtacctgatgtctggaggtatctgccctgtcagccgggacaccata |
| | | gactatttgctttcaaagaatgggagtggcaatgctatcatcatcgtggtcgggggtgcggct |
| | | gagtctctgagctccatgcctggcaagaatgcagtcaccctgeggaaccgcaagggctttgt |
| | | gaaactggccctgegtcatggagctgacctggttcccatctactcctttggagagaatgaagt |
| | | gtacaagcaggtgatcttcgaggagggctcctggggccgatgggtccagaagaagttcca |
| | | gaaatacattggtttcgccccatgcatcttccatggtegaggcctettctcctccgacacctgg |
| | | gggctggtgccctactccaagcccatcaccactgttgtgggagagcccatcaccatcccca |
| | | agctggagcacccaacccagcaagacatcgacctgtaccacaccatgtacatggaggccc |
| | | tggtgaagctcttcgacaagcacaagaccaagttcggccteccggagactgaggtectgga |
| | | ggtgaactgagccagccttcggggccaattccctggaggaaccagctgcaaatcactttttt |
| | | gctctgtaaatttggaagtgtcatgggtgtctgtgggttatttaaaagaaattataacaattttgct |
| | | aaaccattacaatgttaggtcttttttaagaaggaaaaagtcagtatttcaagttetttcacttcca |
| | | gcttgccctgttctaggtggtggctaaatetgggcctaatctgggtggctcagctaacctetctt |
| | | cttcccttcctgaagtgacaaaggaaactcagtcttcttggggaagaaggattgccattagtg |
| | | acttggaccagttagatgattcactttttgcccctagggatgagaggegaaagccacttctcat |
| | | acaagcccctttattgccactaccccacgctegtctagtcctgaaactgcaggaccagtttctc |
| | | tgccaaggggaggagttggagagcacagttgccccgttgtgtgagggcagtagtaggcat |
| | | ctggaatgctccagtttgatetcocttctgccacccctacctcacccctagtcactcatatogga |
| | | gcctggactggcctccaggatgaggatgggggtggcaatgacaccctgcaggggaaagg |
| | | actgccccccatgcaccattgcagggaggatgccgccaccatgagetaggtggagtaact |
| | | ggtttttcttgggtggctgatgacatggatgcagcacagactcagccttggcctggagcacat |
| | | gettactggtggcctcagtttaccttccccagatcctagattctggatgtgaggaagagatccc |
| | | tcttcagaaggggcctggccttctgagcagcagattagttccaaagcaggtggcccccgaa |
| | | cccaagcctcacttttctgtgccttcctgagggggttgggceggggaggaaacccaaccetc |
| | | tcctgtgtgttctgttatctcttgatgagatcattgcaccatgtcagacttttgtatatgccttgaaa |
| | | ataaatgaaagtgagaatcctctaaaaaaaaaaaa |
| 94 | HBV | ctccaccactttccaccaaactcttcaagatcccagagtcagggecetgtactttcctgctggt |
| | Genbank | ggctcaagttccggaacagtaaaccctgctccgactactgcetetcccatategtcaatcttct |
| | Accession | cgaggactggggaccetgtaccgaatatggagagcaccacatcaggattcctaggacccct |
| | No. | gctcgtgttacaggggggtttttcttgttgacaagaatcctcacaataccacagagtctagac |
| | KC315400. | tcgtggtggacttctctcaattttctagggggagcacccacgtgtcctggccaaaatttgcagt |
| | 1 | ccccaacctccaatcactcaccaacctettgtcctccaatttgtcctggttatcgctggatgtgt |
| | | ctgeggogttttatcatettcctettcatectgetgctatgectcatettettgttggttettctggac |
| | | taccaaggtatgttgcccgtttgtcctctacttccaggaacatcaactaccagcaceggaccat |
| | | gcaaaacctgcacaactactgctcaagggacctetatgtttccetcatgttgetgtacaaaacc |
| | | tacggacggaaactgcacctgtattcccatcccatcatcttgggctttcgcaaaatacctatgg |
| | | gagtgggcctcagtccgtttctcttggctcagtttactagtgccatttgltcagtggttcgtagg |
| | | gctttcccccactgtctggctttcagttatatggatgatgtggttttgggggccaagtetgtaca |
| | | acatcttgagtccctttataccgctgttaccaattttcttttatclltgggtatacatttaaaccctca |
| | | caaaacaaaaagatggggatattcccttaacttcatgggatatgtaattgggagttggggcac |
| | | tttgcctcaggaacatattgtacaaaaaatcaagcaatgttttaggaaacttcctgtaaacagg |
| | | cctattgattggaaagtatgtcaacraattgtgggtcttttggggtttgccgcccctttcacgca |
| | | atgtggatatcctgctttaatgcctttatatgcatgtatacaagctaagcaggcttttactttctcg |
| | | ccaacttacaaggcctttctgtgtaaacaatatctgaacctttaccccgttgcteggcaacggt |
| | | caggtctttgccaagtgtttgctgacgcaacccccactggttggggcttggccataggccatc |
| | | agcgcatgcgtggaacctttgtggctcctctgccgatccatactgcggaactcctagcagctt |
| | | gttttgctcgcagccggtctggagcaaaacttateggcaccgacaactetgttgtectetctcg |
| | | gaaatacacctcctttccatggctgctaggatgtgctgccaactggatcctgcgcgggacgt |
| | | cctttgtctacgtcccgteggcgctgaatcccgcggacgacccatctcggggccgtttggga |
| | | ctctaccgtccccttetgegtctgccgttcegcccgaccacggggegcacctetetttacgeg |
| | | gtctccccgtetgtgccttctcatctgceggaccgtgtgcacttegottcacctctgcacgtcg |
| | | catggagaccaccgtgaacgcccacgggaacctgcccaaggtcttgcataagaggactett |
| | | ggactttcagcaatgtcaacgaccgaccttgaggcatacttcaaagactgtgtgtttactgagt |
| | | gggaggagttgggggaggaggttaggttaaaggtctttgtactaggaggctgtaggcataa |
| | | attggtgtgttcaccagcaccatgcaactttttcacctetgcctaatcatotcatgttcatgtccta |
| | | ctgttcaagcctccaagctgtgccttgggtggctttggggcatggacattgacccgtataaag |
| | | aatttggagcttctgtggagttactctcttttttgccttetgacttetttccttctattcgagatctcct |
| | | cgacaccgcctetgetctgtatcgggaggccttagagtctccggaacattgttcacctcacca |
| | | tacggcactcaggcaagcaattctgtgttggggtgagttaatgaatctagccacctgggtgg |
| | | gaagtaatttggaagatccagcatccagggaattagtagtcagctatgtcaacgttaatatgg |
| | | gcctaaaaatcagacaactattgtggtttcacatttectgtcttacttttgggagagaaactgttc |
| | | ttgaatatttggtgtcttttggagtgtggattcgcactcctcctgcatatagaccacaaaatgcc |
| | | cctatcttatcaacacttccggaaactactgttgttagacgaagaggcaggtcccctagaaga |
| | | agaactccctegcctegcagacgaaggtctcaategccgegtegcagaagatetcaatetc |
| | | gggaatctcaatgttagtattccttggacacataaggtgggaaactttacggggetttattettct |
| | | acggtaccttgctttaatcctaaatggcaaactecttettttcctgacattcatttgcaggaggac |
| | | attgttgatagatgtaagcaatttgtggggccccttacagtaaatgaaaacaggagacttaaat |
| | | taattatgcctgctaggttttatcccaatgttactaaatatttgcccttagataaagggatcaaac |
| | | cgtattatccagagtatgtagttaatcattacttccagacgcgacattatttacacactctttgga |
| | | aggggggatcttatataaaagagagtccacacgtagcgcctcattttgegggtcaccatatt |
| | | cttgggaacaagatctacagcatgggaggttggtcttccaaacctcgaaaaggcatgggga |
| | | caaatctttctgtccccaatcccctgggattcttccccgatcatcagttggaccctgcattcaaa |
| | | gccaactcagaaaatccagattgggacctcaacccacacaaggacaactggceggacgcc |
| | | aacaaggtgggagtgggagcattcgggccagggttcacccctcctcatgggggactgttg |
| | | gggtggagccctcaggetcagggcatattcacaacagtgccagcagetectoctcctgcct |
| | | ccaccaateggcagtcaggaaggcagcctactcccttetctccacctctaagagacactcat |
| | | cctcaggccatgcagtggaa |
| 95 | ASO 1 | GalNAc4-ps-GalNAc4-ps-GalNAc4-po-mA-po- |
| | | lnGpslnApslnTpsInApslnApsApsAps(5OH)CpsGps(5m)Cps(5m) |
| | | CpsGps(5m)CpslnApslnGpslnApscp(5m)C |
| 96 | ASO 2 | mA-po- |
| | | lnGpslnApslnTpstnApslnApsApsAps(5OH)CpsGps(5m)Cps(5m) |
| | | CpsGps(5m)CpsInApslnGpslnApscp(5m)C |
| 97 | SARS-COV- | attaaaggtttatacetteccaggtaacaaaccaaccaactttegatetettgtagatetgttetct |
| | 2 genome | aaacgaactttaaaatctgtgtggctgtcacteggctgcatgettagtgcactcacgcagtata |
| | (Genbank | attaataactaattactgtogttgacaggacacgagtaactegtctatcttctgcaggetgctta |
| | Accession | cggtttegtccgtgttgcagcegatcatcagcacatctaggtttcgtcegggtgtgaccgaaa |
| | No. | ggtaagatggagagccttgtccctggtttcaacgagaaaacacacgtccaactcagtttgcct |
| | NC_045512.2) | gttttacaggttcgcgacgtgctcgtacgtggctttggagactccgtggaggaggtcttatca |
| | | gaggcacgtcaacatcttaaagatggcacttgtggcttagtagaagttgaaaaaggcgttttg |
| | | cctcaacttgaacagccctatgtgttcatcaaacgtteggatgctegaactgeacctcatggtc |
| | | atgttatggttgagctggtagcagaactcgaaggcattcagtacggtcgtagtggtgagaca |
| | | cttggtgtccttgtccctcatgtgggegaaataccagtggcttaccgcaaggttettettegtaa |
| | | gaacggtaataaaggagctggtggccatagttacggcgccgatctaaagtcatttgacttag |
| | | gcgacgagcttggcactgatccttatgaagattttcaagaaaactggaacactaaacatagca |
| | | gtggtgttacccgtgaactcatgcgtgagcttaacggaggggcatacactcgctatgtogata |
| | | acaacttetgtggccctgatggctaccctcttgagtgcattaaagaccttctagcacgtgetgg |
| | | taaagcttcatgcactttgtccgaacaactggactttattgacactaagaggggtgtatactgct |
| | | gccgtgaacatgagcatgaaattgcttggtacacggaacgttctgaaaagagctatgaattg |
| | | cagacaccttttgaaattaaattggcaaagaaatttgacaccttcaatggggaatgtccaaattt |
| | | tgtatttcccttaaattccataatcaagactattcaaccaagggttgaaaagaaaaagcttgatg |
| | | gctttatgggtagaattcgatctgtctatccagttgcgtcaccaaatgaatgcaaccaaatgtg |
| | | cctttcaactctcatgaagtgtgatcattgtggtgaaacttcatggcagacgggegattttgtta |
| | | aagccacttgcgaattttgtggcactgagaatttgactaaagaaggtgccactacttgtggtta |
| | | cttaccccaaaatgctgttgttaaaatttattgtccagcatgtcacaattcagaagtaggacctg |
| | | agcatagtcttgccgaataccataatgaatctggottgaaaaccattcttcgtaagggtggtcg |
| | | cactattgcctttggaggetgtgtgttctcttatgttggttgccataacaagtgtgcctattgggtt |
| | | ccacgtgctagcgctaacataggttgtaaccatacaggtgttgttggagaaggttccgaaggt |
| | | cttaatgacaaccttottgaaatactccaaaaagagaaagtcaacatcaatattgttggtgactt |
| | | taaacttaatgaagagatcgccattattttggcatctttttctgcttccacaagtgcttttgtggaa |
| | | actgtgaaaggtttggattataaagcattcaaacaaattgttgaatcctgtggtaattttaaagtt |
| | | acaaaaggaaaagctaaaaaaggtgcctggaatattggtgaacagaaatcaatactgagtc |
| | | ctctttatgcatttgcatcagaggctgctcgtgttgtacgatcaattttctcccgcactettgaaa |
| | | ctgctcaaaattctgtgcgtgttttacagaaggccgctataacaatactagatggaatttcaca |
| | | gtattcactgagactcattgatgctatgatgttcacatctgatttggctactaacaatctagttgta |
| | | atggcctacattacaggtggtgttgttcagttgacttcgcagtggctaactaacatctttggcac |
| | | tgtttatgaaaaactcaaacccgtccttgattggcttgaagagaagtttaaggaaggtgtagag |
| | | tttcttagagacggttgggaaattgttaaatttatctcaacctgtgcttgtgaaattgtcggtgga |
| | | caaattgtcacctgtgcaaaggaaattaaggagagtgttcagacattctttaagottgtaaata |
| | | aatttttggctttgtgtgctgactctatcattattggtggagctaaacttaaagccttgaatttaggt |
| | | gaaacatttgtcacgcactcaaagggattgtacagaaagtgtgttaaatccagagaagaaac |
| | | tggcctactcatgcctctaaaagccccaaaagaaattatettettagagggagaaacacttcc |
| | | cacagaagtgttaacagaggaagttgtcttgaaaactggtgatttacaaccattagaacaacc |
| | | tactagtgaagctgttgaagctccattggttggtacaccagtttgtattaacgggcttatgttgct |
| | | cgaaatcaaagacacagaaaagtactgtgcccttgcacctaatatgatggtaacaaacaata |
| | | ccttcacactcaaaggeggtgcaccaacaaaggttacttttggtgatgacactgtgatagaag |
| | | tgcaaggttacaagagtgtgaatatcacttttgaacttgatgaaaggattgataaagtacttaat |
| | | gagaagtgetctgcctatacagttgaactcggtacagaagtaaatgagttcgcctgtgttgtg |
| | | gcagatgctgtcataaaaactttgcaaccagtatctgaattacttacaccactgggcattgattt |
| | | agatgagtggagtatggctacatactacttatttgatgagtctggtgagtttaaattggcttcac |
| | | atatgtattgttotttctaccctccagatgaggatgaagaagaaggtgattgtgaagaagaaga |
| | | gtttgagccatcaactcaatatgagtatggtactgaagatgattaccaaggtaaacctttggaa |
| | | tttggtgccacttctgctgctcttcaacctgaagaagagcaagaagaagattggttagatgatg |
| | | atagtcaacaaactgttggtcaacaagacggcagtgaggacaatcagacaactactattcaa |
| | | acaattgttgaggttcaacctcaattagagatggaacttacaccagttgttcagactattgaagt |
| | | gaatagttttagtggttatttaaaacttactgacaatgtatacattaaaaatgcagacattgtgga |
| | | agaagctaaaaaggtaaaaccaacagtggttgttaatgcagccaatgtttaccttaaacatgg |
| | | aggaggtgttgcaggagccttaaataaggctactaacaatgccatgcaagttgaatctgatg |
| | | attacatagctactaatggaccacttaaagtgggtggtagttgtgttttaageggacacaatott |
| | | gctaaacactgtcttcatgttgteggcccaaatgttaacaaaggtgaagacattcaacttcttaa |
| | | gagtgcttatgaaaattttaatcagcacgaagttctacttgcaccattattatcagctggtattttt |
| | | ggtgctgaccctatacattctttaagagtttgtgtagatactgttcgcacaaatgtctacttagct |
| | | gtctttgataaaaatctctatgacaaacttgtttcaagctttttggaaatgaagagtgaaaagca |
| | | agttgaacaaaagatcgctgagattcctaaagaggaagttaagccatttataactgaaagtaa |
| | | accttcagttgaacagagaaaacaagatgataagaaaatcaaagcttgtgttgaagaagttac |
| | | aacaactctggaagaaactaagttcctcacagaaaacttgttactttatattgacattaatggca |
| | | atcttcatccagattctgccactcttgttagtgacattgacatcactttcttaaagaaagatgetc |
| | | catatatagtgggtgatgttgttcaagagggtgttttaactgctgtggttatacctactaaaaag |
| | | gctggtggcactactgaaatgctagcgaaagctttgagaaaagtgccaacagacaattatat |
| | | aaccacttaccogggtcagggtttaaatggttacactgtagaggaggcaaagacagtgctta |
| | | aaaagtgtaaaagtgccttttacattctaccatctattatctctaatgagaagcaagaaattcttg |
| | | gaactgtttcttggaatttgcgagaaatgcttgcacatgcagaagaaacacgcaaattaatgc |
| | | ctgtctgtgtggaaactaaagccatagtttcaactatacagcgtaaatataagggtattaaaata |
| | | caagagggtgtggttgattatggtgctagattttacttttacaccagtaaaacaactgtagegtc |
| | | acttatcaacacacttaacgatctaaatgaaactcttgttacaatgccacttggctatgtaacac |
| | | atggcttaaatttggaagaagctgctcggtatatgagatctctcaaagtgccagctacagtttct |
| | | gtttcttcacctgatgctgttacagegtataatggttatcttacttcttcttctaaaacacctgaag |
| | | aacattttattgaaaccatctcacttgctggttcctataaagattggtcctattctggacaatctac |
| | | acaactaggtatagaatttcttaagagaggtgataaaagtgtatattacactagtaatcctacca |
| | | cattccacctagatggtgaagttatcacctttgacaatcttaagacacttctttctttgagagaag |
| | | tgaggactattaaggtgtttacaacagtagacaacattaacctccacacgcaagttgtggaca |
| | | tgtcaatgacatatggacaacagtttggtccaacttatttggatggagctgatgttactaaaata |
| | | aaacctcataattcacatgaaggtaaaacattttatgttttacctaatgatgacactctacgtgtt |
| | | gaggcttttgagtactaccacacaactgatcctagttttctgggtaggtacatgtcagcattaaa |
| | | tcacactaaaaagtggaaatacccacaagttaatggtttaacttctattaaatgggcagataac |
| | | aactgttatcttgccactgcattgttaacactccaacaaatagagttgaagtttaatccacctget |
| | | ctacaagatgettattacagagcaagggctggtgaagctgctaacttttgtgcacttatettagc |
| | | ctactgtaataagacagtaggtgagttaggtgatgttagagaaacaatgagttacttgtttcaa |
| | | catgccaatttagattcttgcaaaagagtcttgaacgtggtgtgtaaaacttgtggacaacagc |
| | | agacaaccettaagggtgtagaagctgttatgtacatgggcacactttcttatgaacaatttaa |
| | | gaaaggtgttcagataccttgtacgtgtggtaaacaagctacaaaatatctagtacaacagga |
| | | gtcaccttttgttatgatgtcagcaccacctgotcagtatgaacttaagcatggtacatttacttg |
| | | tgctagtgagtacactggtaattaccagtgtggtcactataaacatataacttctaaagaaactt |
| | | tgtattgcatagacggtgctttacttacaaagtcctcagaatacaaaggtcctattacggatgttt |
| | | tctacaaagaaaacagttacacaacaaccataaaaccagttacttataaattggatggtgttgtt |
| | | tgtacagaaattgaccctaagttggacaattattataagaaagacaattcttatttcacagagca |
| | | accaattgatcttgtaccaaaccaaccatatccaaacgcaagcttcgataattttaagtttgtat |
| | | gtgataatatcaaatttgctgatgatttaaaccagttaactggttataagaaacctgottcaaga |
| | | gagcttaaagttacatttttccctgacttaaatggtgatgtggtggctattgattataaacactac |
| | | acaccctcttttaagaaaggagctaaattgttacataaacctattgtttggcatgttaacaatgca |
| | | actaataaagccacgtataaaccaaatacctggtgtatacgttgtctttggagcacaaaacca |
| | | gttgaaacatcaaattcgtttgatgtactgaagtcagaggacgcgcagggaatggataatctt |
| | | gcctgcgaagatctaaaaccagtctctgaagaagtagtggaaaatcctaccatacagaaag |
| | | acgttcttgagtgtaatgtgaaaactaccgaagttgtaggagacattatacttaaaccagcaaa |
| | | taatagtttaaaaattacagaagaggttggccacacagatctaatggctgcttatgtagacaat |
| | | tctagtcttactattaagaaacctaatgaattatctagagtattaggtttgaaaacccttgctactc |
| | | atggtttagctgctgttaatagtgtcccttgggatactatagctaattatgctaagccttttcttaa |
| | | caaagttgttagtacaactactaacatagttacacggtgtttaaaccgtgtttgtactaattatatg |
| | | ccttatttctttactttattgctacaattgtgtacttttactagaagtacaaattctagaattaaagca |
| | | tctatgccgactactatagcaaagaatactgttaagagtgtcggtaaattttgtctagaggcttc |
| | | atttaattatttgaagtcacctaatttttctaaactgataaatattataatttggtttttactattaagtg |
| | | tttgcctaggttctttaatctactcaaccgctgctttaggtgttttaatgtctaatttaggcatgcctt |
| | | cttactgtactggttacagagaaggctatttgaactctactaatgtcactattgcaacctactgta |
| | | ctggttctataccttgtagtgtttgtcttagtggtttagattetttagacacctatccttetttagaaa |
| | | ctatacaaattaccatttcatcttttaaatgggatttaactgottttggettagttgcagagtggtttt |
| | | tggcatatattcttttcactaggtttttctatgtacttggattggctgcaatcatgcaattgtttttca |
| | | gctattttgcagtacattttattagtaattcttggcttatgtggttaataattaatcttgtacaaatgg |
| | | ccccgatttcagctatggttagaatgtacatcttetttgcatcattttattatgtatggaaaagttat |
| | | gtgcatgttgtagacggttgtaattcatcaacttgtatgatgtgttacaaacgtaatagagcaac |
| | | aagagtcgaatgtacaactattgttaatggtgttagaaggtccttttatgtctatgctaatggag |
| | | gtaaaggcttttgcaaactacacaattggaattgtgttaattgtgatacattctgtgetggtagta |
| | | catttattagtgatgaagttgcgagagacttgtcactacagtttaaaagaccaataaatcctact |
| | | gaccagtcttcttacatcgttgatagtgttacagtgaagaatggttccatccatctttactttgata |
| | | aagctggtcaaaagacttatgaaagacattctctctctcattttgttaacttagacaacctgaga |
| | | gctaataacactaaaggttcattgcctattaatgttatagtttttgatggtaaatcaaaatgtgaa |
| | | gaatcatctgcaaaatcagcgtctgtttactacagtcagcttatgtgtcaacctatactgttacta |
| | | gatcaggcattagtgtctgatgttggtgatagtgcggaagttgcagttaaaatgtttgatgetta |
| | | cgttaatacgttttcatcaacttttaacgtaccaatggaaaaactcaaaacactagttgcaactg |
| | | cagaagctgaacttgcaaagaatgtgtccttagacaatgtcttatctacttttatttcagcagete |
| | | ggcaagggtttgttgattcagatgtagaaactaaagatgttgttgaatgtcttaaattgtcacatc |
| | | aatctgacatagaagttactggcgatagttgtaataactatatgctcacctataacaaagttgaa |
| | | aacatgacaccccgtgaccttggtgettgtattgactgtagtgcgcgtcatattaatgegcagg |
| | | tagcaaaaagtcacaacattgctttgatatggaacgttaaagatttcatgtcattgtctgaacaa |
| | | ctacgaaaacaaatacgtagtgctgctaaaaagaataacttaccttttaagttgacatgtgcaa |
| | | ctactagacaagttgttaatgttgtaacaacaaagatagcacttaagggtggtaaaattgttaat |
| | | aattggttgaagcagttaattaaagttacacttgtgttcctttttgttgctgctattttctatttaataa |
| | | cacctgttcatgtcatgtctaaacatactgacttttcaagtgaaatcataggatacaaggctatt |
| | | gatggtggtgtcactcgtgacatagcatctacagatacttgttttgctaacaaacatgctgatttt |
| | | gacacatggtttagccagcgtggtggtagttatactaatgacaaagcttgcccattgattgctg |
| | | cagtcataacaagagaagtgggttttgtcgtgcctggtttgcctggcacgatattacgcacaa |
| | | ctaatggtgactttttgcatttcttacctagagtttttagtgcagttggtaacatctgttacacacca |
| | | tcaaaacttatagagtacactgactttgcaacatcagcttgtgttttggctgctgaatgtacaatt |
| | | tttaaagatgcttctggtaagccagtaccatattgttatgataccaatgtactagaaggttctgtt |
| | | gcttatgaaagtttacgccctgacacacgttatgtgctcatggatggctctattattcaatttect |
| | | aacacctaccttgaaggttctgttagagtggtaacaacttttgattotgagtactgtaggcacg |
| | | gcacttgtgaaagatcagaagctggtgtttgtgtatctactagtggtagatgggtacttaacaat |
| | | gattattacagatctttaccaggagttttctgtggtgtagatgctgtaaatttacttactaatatgttt |
| | | acaccactaattcaacctattggtgctttggacatatcagcatctatagtagctggtggtattgta |
| | | gctategtagtaacatgccttgcctactattttatgaggtttagaagagcttttggtgaatacagt |
| | | catgtagttgcctttaatactttactattccttatgtcattcactgtactctgtttaacaccagtttact |
| | | cattcttacctggtgtttattetgttatttacttgtacttgacattttatettactaatgatgtttctttttt |
| | | agcacatattcagtggatggttatgttcacacctttagtacctttctggataacaattgettatatc |
| | | atttgtatttccacaaagcatttctattggttotttagtaattacctaaagagacgtgtagtctttaat |
| | | ggtgtttcctttagtacttttgaagaagctgcgctgtgcacctttttgttaaataaagaaatgtatc |
| | | taaagttgogtagtgatgtgctattacctcttacgcaatataatagatacttagctetttataataa |
| | | gtacaagtattttagtggagcaatggatacaactagctacagagaagetgettgttgtcatctc |
| | | gcaaaggctctcaatgacttcagtaactcaggttctgatgttctttaccaaccaccacaaacct |
| | | ctatcacctcagctgttttgcagagtggttttagaaaaatggcattcccatctggtaaagttgag |
| | | ggttgtatggtacaagtaacttgtggtacaactacacttaacggtctttggcttgatgacgtagt |
| | | ttactgtccaagacatgtgatctgcacctctgaagacatgettaaccctaattatgaagatttact |
| | | cattcgtaagtctaatcataatttettggtacaggctggtaatgttcaactcagggttattggaca |
| | | ttctatgcaaaattgtgtacttaagcttaaggttgatacagccaatcctaagacacctaagtata |
| | | agtttgttcgcattcaaccaggacagactttttcagtgttagcttgttacaatggttcaccatctg |
| | | gtgtttaccaatgtgctatgaggcccaatttcactattaagggttcattccttaatggttcatgtg |
| | | gtagtgttggttttaacatagattatgactgtgtctctttttgttacatgcaccatatggaattacca |
| | | actggagttcatgetggcacagacttagaaggtaacttttatggaccttttgttgacaggcaaa |
| | | cagcacaagcagctggtacggacacaactattacagttaatgttttagcttggttgtacgctgc |
| | | tgttataaatggagacaggtggtttctcaatcgatttaccacaactcttaatgactttaaccttgt |
| | | ggctatgaagtacaattatgaacctctaacacaagaccatgttgacatactaggacctetttct |
| | | gctcaaactggaattgccgttttagatatgtgtgcttcattaaaagaattactgcaaaatggtat |
| | | gaatggacgtaccatattgggtagtgctttattagaagatgaatttacaccttttgatgttgttag |
| | | acaatgctcaggtgttactttccaaagtgcagtgaaaagaacaatcaagggtacacaccact |
| | | ggttgttactcacaattttgacttcacttttagttttagtccagagtactcaatggtctttgttctttttt |
| | | ttgtatgaaaatgcctttttaccttttgctatgggtattattgctatgtctgcttttgcaatgatgtttg |
| | | tcaaacataagcatgcatttctctgttttttttgttaccttctcttgccactgtagcttattttaatat |
| | | ggtctatatgcctgctagttgggtgatgcgtattatgacatggttggatatggttgatactagttt |
| | | gtctggttttaagctaaaagactgtgttatgtatgcatcagctgtagtgttactaatccttatgaca |
| | | gcaagaactgtgtatgatgatggtgctaggagagtgtggacacttatgaatgtcttgacactc |
| | | gtttataaagtttattatggtaatgctttagatcaagccatttccatgtgggctettataatctetgtt |
| | | acttctaactactcaggtgtagttacaactgtcatgtttttggccagaggtattgtttttatgtgtgt |
| | | tgagtattgccctattttcttcataactggtaatacacttcagtgtataatgctagtttattgtttctta |
| | | ggctatttttgtacttgttactttggcctcttttgtttactcaaccgctactttagactgactcttggt |
| | | gtttatgattacttagtttctacacaggagtttagatatatgaattcacagggactacteccaccc |
| | | aagaatagcatagatgccttcaaactcaacattaaattgttgggtgttggtggcaaaccttgtat |
| | | caaagtagccactgtacagtctaaaatgtcagatgtaaagtgcacatcagtagtettactctca |
| | | gttttgcaacaactcagagtagaatcatcatctaaattgtgggctcaatgtgtccagttacacaa |
| | | tgacattctcttagctaaagatactactgaagectttgaaaaaatggtttcactactttctgttttg |
| | | ctttccatgcagggtgctgtagacataaacaagctttgtgaagaaatgctggacaacagggc |
| | | aaccttacaagctatagcctcagagtttagttccettecatcatatgcagcttttgctactgctca |
| | | agaagcttatgagcaggctgttgctaatggtgattctgaagttgttcttaaaaagttgaagaagt |
| | | ctttgaatgtggctaaatctgaatttgaccgtgatgcagccatgcaacgtaagttggaaaagat |
| | | ggctgatcaagctatgacccaaatgtataaacaggctagatctgaggacaagagggcaaaa |
| | | gttactagtgctatgcagacaatgcttttcactatgcttagaaagttggataatgatgcactcaa |
| | | caacattatcaacaatgcaagagatggttgtgttcccttgaacataatacctcttacaacagca |
| | | gccaaactaatggttgtcataccagactataacacatataaaaatacgtgtgatggtacaacat |
| | | ttacttatgcatcagcattgtgggaaatccaacaggttgtagatgcagatagtaaaattgttcaa |
| | | cttagtgaaattagtatggacaattcacctaatttagcatggcctcttattgtaacagctttaagg |
| | | gccaattctgctgtcaaattacagaataatgagcttagtcctgttgcactacgacagatgtcttg |
| | | tgctgccggtactacacaaactgcttgcactgatgacaatgcgttagcttactacaacacaac |
| | | aaagggaggtaggtttgtacttgcactgttatccgatttacaggatttgaaatgggctagattcc |
| | | ctaagagtgatggaactggtactatctatacagaactggaaccaccttgtaggtttgttacaga |
| | | cacacctaaaggtcctaaagtgaagtatttatactttattaaaggattaaacaacctaaatagag |
| | | gtatggtacttggtagtttagctgccacagtacgtctacaagctggtaatgcaacagaagtgc |
| | | ctgccaattcaactgtattatctttctgtgcttttgctgtagatgctgctaaagcttacaaagattat |
| | | ctagctagtgggggacaaccaatcactaattgtgttaagatgttgtgtacacacactggtactg |
| | | gtcaggcaataacagttacaccggaagccaatatggatcaagaatcctttggtggtgcatcgt |
| | | gttgtctgtactgccgttgccacatagatcatccaaatcctaaaggattttgtgacttaaaaggt |
| | | aagtatgtacaaatacctacaacttgtgctaatgaccctgtgggttttacacttaaaaacacagt |
| | | ctgtaccgtctgcggtatgtggaaaggttatggctgtagttgtgatcaactccgcgaacccat |
| | | gcttcagtcagctgatgcacaatcgtttttaaacgggtttgcggtgtaagtgcagcccgtctta |
| | | caccgtgoggcacaggcactagtactgatgtcgtatacagggcttttgacatctacaatgata |
| | | aagtagctggttttgctaaattcctaaaaactaattgttgtogcttccaagaaaaggacgaaga |
| | | tgacaatttaattgattcttactttgtagttaagagacacactttctctaactaccaacatgaagaa |
| | | acaatttataatttacttaaggattgtccagctgttgctaaacatgacttctttaagtttagaatag |
| | | acggtgacatggtaccacatatatcacgtcaacgtcttactaaatacacaatggcagaccteg |
| | | tctatgctttaaggcattttgatgaaggtaattgtgacacattaaaagaaatacttgtcacataca |
| | | attgttgtgatgatgattatttcaataaaaaggactggtatgattttgtagaaaacccagatatatt |
| | | acgcgtatacgccaacttaggtgaacgtgtacgccaagctttgttaaaaacagtacaattctgt |
| | | gatgccatgcgaaatgctggtattgttggtgtactgacattagataatcaagatctcaatggta |
| | | actggtatgatttcggtgatttcatacaaaccacgccaggtagtggagttcctgttgtagattctt |
| | | attattcattgttaatgcctatattaaccttgaccagggctttaactgcagagtcacatgttgaca |
| | | ctgacttaacaaagccttacattaagtgggatttgttaaaatatgacttcacggaagagaggtt |
| | | aaaactctttgaccgttattttaaatattgggatcagacataccacccaaattgtgttaactgtttg |
| | | gatgacagatgcattctgcattgtgcaaactttaatgttttattctctacagtgttcccacctacaa |
| | | gttttggaccactagtgagaaaaatatttgttgatggtgttccatttgtagtttcaactggatacc |
| | | acttcagagagctaggtgttgtacataatcaggatgtaaacttacatagctctagacttagtttta |
| | | aggaattacttgtgtatgctgctgaccctgctatgcacgctgcttctggtaatctattactagata |
| | | aacgcactacgtgettttcagtagctgcacttactaacaatgttgcttttcaaactgtcaaaccc |
| | | ggtaattttaacaaagacttctatgactttgctgtgtctaagggtttctttaaggaaggaagttct |
| | | gttgaattaaaacacttcttctttgctcaggatggtaatgetgctatcagegattatgactactat |
| | | cgttataatctaccaacaatgtgtgatatcagacaactactatttgtagttgaagttgttgataag |
| | | tactttgattgttacgatggtggctgtattaatgctaaccaagtcatcgtcaacaacctagacaa |
| | | atcagctggttttccatttaataaatggggtaaggctagactttattatgattcaatgagttatga |
| | | ggatcaagatgcacttttegcatatacaaaacgtaatgtcatccctactataactcaaatgaatc |
| | | ttaagtatgccattagtgcaaagaatagagctcgcaccgtagctggtgtctctatctgtagtact |
| | | atgaccaatagacagtttcatcaaaaattattgaaatcaatagcegccactagaggagctact |
| | | gtagtaattggaacaagcaaattctatggtggttggcacaacatgttaaaaactgtttatagtga |
| | | tgtagaaaaccctcaccttatgggttgggattatcctaaatgtgatagagccatgcctaacatg |
| | | cttagaattatggcctcacttgttcttgctcgcaaacatacaacgtgttgtagcttgtcacacegt |
| | | ttctatagattagctaatgagtgtgctcaagtattgagtgaaatggtcatgtgtggcggttcact |
| | | atatgttaaaccaggtggaacctcatcaggagatgccacaactgcttatgctaatagtgttttta |
| | | acatttgtcaagctgtcacggccaatgttaatgcacttttatctactgatggtaacaaaattgcc |
| | | gataagtatgtccgcaatttacaacacagactttatgagtgtctctatagaaatagagatgttga |
| | | cacagactttgtgaatgagttttacgcatatttgcgtaaacatttctcaatgatgatactctctgac |
| | | gatgctgttgtgtgtttcaatagcacttatgcatctcaaggtctagtggctagcataaagaacttt |
| | | aagtcagttctttattatcaaaacaatgtttttatgtctgaagcaaaatgttggactgagactgac |
| | | cttactaaaggacctcatgaattttgetctcaacatacaatgctagttaaacagggtgatgatta |
| | | tgtgtaccttccttacccagatccatcaagaatcctaggggccggctgttttgtagatgatatcg |
| | | taaaaacagatggtacacttatgattgaacggttcgtgtetttagctatagatgcttacccactta |
| | | ctaaacatcctaatcaggagtatgctgatgtctttcatttgtacttacaatacataagaaagctac |
| | | atgatgagttaacaggacacatgttagacatgtattctgttatgcttactaatgataacacttcaa |
| | | ggtattgggaacctgagttttatgaggctatgtacacacegcatacagtcttacaggctgttgg |
| | | ggcttgtgttctttgcaattcacagacttcattaagatgtggtgcttgcatacgtagaccattctta |
| | | tgttgtaaatgetgttacgaccatgtcatatcaacatcacataaattagtcttgtctgttaatccgt |
| | | atgtttgcaatgctccaggttgtgatgtcacagatgtgactcaactttacttaggaggtatgagc |
| | | tattattgtaaatcacataaaccacccattagttttccattgtgtgctaatggacaagtttttggttt |
| | | atataaaaatacatgtgttggtagcgataatgttactgactttaatgcaattgcaacatgtgactg |
| | | gacaaatgctggtgattacattttagctaacacctgtactgaaagactcaagctttttgcagca |
| | | gaaacgctcaaagctactgaggagacatttaaactgtcttatggtattgctactgtacgtgaag |
| | | tgctgtctgacagagaattacatctttcatgggaagttggtaaacctagaccaccacttaaccg |
| | | aaattatgtctttactggttatcgtgtaactaaaaacagtaaagtacaaataggagagtacacct |
| | | ttgaaaaaggtgactatggtgatgctgttgtttacegaggtacaacaacttacaaattaaatgtt |
| | | ggtgattattttgtgctgacatcacatacagtaatgccattaagtgcacctacactagtgccaca |
| | | agagcactatgttagaattactggcttatacccaacactcaatatctcagatgagttttctagca |
| | | atgttgcaaattatcaaaaggttggtatgcaaaagtattctacactccagggaccacctggtac |
| | | tggtaagagtcattttgctattggcctagetctctactacccttctgctcgcatagtgtatacagc |
| | | ttgctctcatgccgctgttgatgcactatgtgagaaggcattaaaatatttgcctatagataaat |
| | | gtagtagaattatacctgcacgtgctcgtgtagagtgttttgataaattcaaagtgaattcaaca |
| | | ttagaacagtatgtcttttgtactgtaaatgcattgcctgagacgacagcagatatagttgtcttt |
| | | gatgaaatttcaatggccacaaattatgatttgagtgttgtcaatgccagattacgtgctaagca |
| | | ctatgtgtacattggcgaccctgctcaattacctgcaccacgcacattgctaactaagggcac |
| | | actagaaccagaatatttcaattcagtgtgtagacttatgaaaactataggtccagacatgttcc |
| | | tcggaacttgtcggegttgtcctgctgaaattgttgacactgtgagtgctttggtttatgataata |
| | | agcttaaagcacataaagacaaatcagctcaatgctttaaaatgttttataagggtgttatcacg |
| | | catgatgtttcatctgcaattaacaggccacaaataggegtggtaagagaattccttacacgta |
| | | accctgcttggagaaaagctgtctttatttcaccttataattcacagaatgctgtagcctcaaag |
| | | attttgggactaccaactcaaactgttgattcatcacagggctcagaatatgactatgtcatatt |
| | | cactcaaaccactgaaacagctcactcttgtaatgtaaacagatttaatgttgctattaccagag |
| | | caaaagtaggcatactttgcataatgtctgatagagacctttatgacaagttgcaatttacaagt |
| | | cttgaaattccacgtaggaatgtggcaactttacaagctgaaaatgtaacaggactctttaaag |
| | | attgtagtaaggtaatcactgggttacatcctacacaggcacctacacacctcagtgttgaca |
| | | ctaaattcaaaactgaaggtttatgtgttgacatacctggcatacctaaggacatgacctatag |
| | | aagactcatctctatgatgggttttaaaatgaattatcaagttaatggttaccctaacatgtttatc |
| | | acccgcgaagaagctataagacatgtacgtgcatggattggcttcgatgtcgaggggtgtca |
| | | tgctactagagaagctgttggtaccaatttacctttacagetaggtttttctacaggtgttaacct |
| | | agttgctgtacctacaggttatgttgatacacctaataatacagatttttccagagttagtgctaa |
| | | accaccgcctggagatcaatttaaacacctcataccacttatgtacaaaggacttccttggaat |
| | | gtagtgcgtataaagattgtacaaatgttaagtgacacacttaaaaatctctctgacagagtcg |
| | | tatttgtcttatgggcacatggctttgagttgacatctatgaagtattttgtgaaaataggacctg |
| | | agcgcacctgttgtctatgtgatagacgtgccacatgcttttccactgcttcagacacttatgcc |
| | | tgttggcatcattctattggatttgattacgtctataatccgtttatgattgatgttcaacaatgggg |
| | | ttttacaggtaacctacaaagcaaccatgatctgtattgtcaagtccatggtaatgcacatgtag |
| | | ctagttgtgatgcaatcatgactaggtgtctagctgtccacgagtgctttgttaagcgtgttgac |
| | | tggactattgaatatcctataattggtgatgaactgaagattaatgcggcttgtagaaaggttca |
| | | acacatggttgttaaagctgcattattagcagacaaattcccagttettcacgacattggtaacc |
| | | ctaaagctattaagtgtgtacctcaagctgatgtagaatggaagttctatgatgcacagccttgt |
| | | agtgacaaagcttataaaatagaagaattattctattcttatgccacacattctgacaaattcaca |
| | | gatggtgtatgcctattttggaattgcaatgtcgatagatatcctgctaattccattgtttgtagatt |
| | | tgacactagagtgctatctaaccttaacttgcctggttgtgatggtggcagtttgtatgtaaataa |
| | | acatgcattccacacaccagcttttgataaaagtgottttgttaatttaaaacaattaccatttttct |
| | | attactctgacagtccatgtgagtctcatggaaaacaagtagtgtcagatatagattatgtacc |
| | | actaaagtctgctacgtgtataacacgttgcaatttaggtggtgctgtctgtagacatcatgcta |
| | | atgagtacagattgtatctcgatgcttataacatgatgatctcagctggctttagcttgtgggttt |
| | | acaaacaatttgatacttataacctctggaacacttttacaagacttcagagtttagaaaatgtg |
| | | gcttttaatgttgtaaataagggacactttgatggacaacagggtgaagtaccagtttctatcat |
| | | taataacactgtttacacaaaagttgatggtgttgatgtagaattgtttgaaaataaaacaacatt |
| | | acctgttaatgtagcatttgagctttgggctaagcgcaacattaaaccagtaccagaggtgaa |
| | | aatactcaataatttgggtgtggacattgctgctaatactgtgatctgggactacaaaagagat |
| | | gctccagcacatatatctactattggtgtttgttctatgactgacatagccaagaaaccaactga |
| | | aacgatttgtgcaccactcactgtcttttttgatggtagagttgatggtcaagtagacttatttag |
| | | aaatgcccgtaatggtgttcttattacagaaggtagtgttaaaggtttacaaccatctgtaggtc |
| | | ccaaacaagctagtottaatggagtcacattaattggagaagccgtaaaaacacagttcaatt |
| | | attataagaaagttgatggtgttgtccaacaattacctgaaacttactttactcagagtagaaatt |
| | | tacaagaatttaaacccaggagtcaaatggaaattgatttcttagaattagctatggatgaattc |
| | | attgaacggtataaattagaaggctatgccttcgaacatatcgtttatggagattttagtcatagt |
| | | cagttaggtggtttacatctactgattggactagctaaacgttttaaggaatcaccttttgaatta |
| | | gaagattttattcctatggacagtacagttaaaaactatttcataacagatgcgcaaacaggttc |
| | | atctaagtgtgtgtgttctgttattgatttattacttgatgattttgttgaaataataaaatcccaaga |
| | | tttatctgtagtttctaaggttgtcaaagtgactattgactatacagaaatttcatttatgctttggtg |
| | | taaagatggccatgtagaaacattttacccaaaattacaatctagtcaagcgtggcaaccggg |
| | | tgttgctatgcctaatctttacaaaatgcaaagaatgctattagaaaagtgtgaccttcaaaatta |
| | | tggtgatagtgcaacattacctaaaggcataatgatgaatgtcgcaaaatatactcaactgtgt |
| | | caatatttaaacacattaacattagctgtaccctataatatgagagttatacattttggtgctggtt |
| | | ctgataaaggagttgcaccaggtacagetgttttaagacagtggttgcctacgggtacgetgc |
| | | ttgtcgattcagatcttaatgactttgtctctgatgcagattcaactttgattggtgattgtgcaact |
| | | gtacatacagctaataaatgggatctcattattagtgatatgtacgaccctaagactaaaaatgt |
| | | tacaaaagaaaatgactctaaagagggttttttcacttacatttgtgggtttatacaacaaaagc |
| | | tagctcttggaggttccgtggctataaagataacagaacattcttggaatgctgatctttataag |
| | | ctcatgggacacttcgcatggtggacagcctttgttactaatgtgaatgcgtcatcatctgaag |
| | | catttttaattggatgtaattatcttggcaaaccacgcgaacaaatagatggttatgtcatgcatg |
| | | caaattacatattttggaggaatacaaatccaattcagttgtcttcctattctttatttgacatgagt |
| | | aaatttccccttaaattaaggggtactgctgttatgtctttaaaagaaggtcaaatcaatgatatg |
| | | attttatctcttcttagtaaaggtagacttataattagagaaaacaacagagttgttatttctagtg |
| | | atgttcttgttaacaactaaacgaacaatgtttgtttttcttgttttattgccactagtctctagtcag |
| | | tgtgttaatcttacaaccagaactcaattaccccctgcatacactaattctttcacacgtggtgttt |
| | | attaccctgacaaagttttcagatcctcagttttacattcaactcaggacttgttcttacctttctttt |
| | | ccaatgttacttggttccatgctatacatgtctctgggaccaatggtactaagaggtttgataac |
| | | cctgtcctaccatttaatgatggtgtttattttgcttccactgagaagtctaacataataagaggc |
| | | tggatttttggtactactttagattcgaagacccagtccctacttattgttaataacgctactaatg |
| | | ttgttattaaagtctgtgaatttcaattttgtaatgatccatttttgggtgtttattaccacaaaaaca |
| | | acaaaagttggatggaaagtgagttcagagtttattctagtgcgaataattgcacttttgaatat |
| | | gtctctcagccttttcttatggaccttgaaggaaaacagggtaatttcaaaaatcttagggaattt |
| | | gtgtttaagaatattgatggttattttaaaatatattctaagcacacgcctattaatttagtgcgtga |
| | | tctccctcagggtttttcggctttagaaccattggtagatttgccaataggtattaacatcactag |
| | | gtttcaaactttacttgctttacatagaagttatttgactcctggtgattcttcttcaggttggacag |
| | | ctggtgctgcagcttattatgtgggttatcttcaacctaggacttttctattaaaatataatgaaaa |
| | | tggaaccattacagatgctgtagactgtgcacttgaccctctctcagaaacaaagtgtacgttg |
| | | aaatccttcactgtagaaaaaggaatctatcaaacttctaactttagagtccaaccaacagaat |
| | | ctattgttagatttcctaatattacaaacttgtgcccttttggtgaagtttttaacgccaccagattt |
| | | gcatctgtttatgcttggaacaggaagagaatcagcaactgtgttgctgattattetgtcctatat |
| | | aattccgcatcattttccacttttaagtgttatggagtgtctcctactaaattaaatgatctctgcttt |
| | | actaatgtctatgcagattcatttgtaattagaggtgatgaagtcagacaaatcgctccagggc |
| | | aaactggaaagattgctgattataattataaattaccagatgattttacaggctgcgttatagctt |
| | | ggaattctaacaatcttgattctaaggttggtggtaattataattacctgtatagattgtttaggaa |
| | | gtctaatctcaaaccttttgagagagatatttcaactgaaatctatcaggccggtagcacacctt |
| | | gtaatggtgttgaaggttttaattgttactttcctttacaatcatatggtttccaacccactaatggt |
| | | gttggttaccaaccatacagagtagtagtactttcttttgaacttctacatgcaccagcaactgtt |
| | | tgtggacctaaaaagtctactaatttggttaaaaacaaatgtgtcaatttcaacttcaatggttta |
| | | acaggcacaggtgttcttactgagtctaacaaaaagtttctgcctttccaacaatttggcagag |
| | | acattgctgacactactgatgctgtccgtgatccacagacacttgagattcttgacattacacc |
| | | atgttcttttggtggtgtcagtgttataacaccaggaacaaatacttctaaccaggttgetgttett |
| | | tatcaggatgttaactgcacagaagtccctgttgctattcatgcagatcaacttactcctacttg |
| | | gcgtgtttattctacaggttctaatgtttttcaaacacgtgcaggctgtttaataggggctgaaca |
| | | tgtcaacaactcatatgagtgtgacatacccattggtgcaggtatatgcgctagttatcagact |
| | | cagactaattctccteggegggcacgtagtgtagctagtcaatccatcattgcctacactatgt |
| | | cacttggtgcagaaaattcagttgcttactctaataactctattgccatacccacaaattttactat |
| | | tagtgttaccacagaaattctaccagtgtctatgaccaagacatcagtagattgtacaatgtac |
| | | atttgtggtgattcaactgaatgcagcaatcttttgttgcaatatggcagtttttgtacacaattaa |
| | | accgtgctttaactggaatagctgttgaacaagacaaaaacacccaagaagtttttgcacaag |
| | | tcaaacaaatttacaaaacaccaccaattaaagattttggtggttttaatttttcacaaatattacc |
| | | agatccatcaaaaccaagcaagaggtcatttattgaagatctacttttcaacaaagtgacactt |
| | | gcagatgctggcttcatcaaacaatatggtgattgccttggtgatattgctgctagagacctca |
| | | tttgtgcacaaaagtttaacggccttactgttttgccacctttgetcacagatgaaatgattgetc |
| | | aatacacttctgcactgttagcgggtacaatcacttctggttggacctttggtgcaggtgctgc |
| | | attacaaataccatttgctatgcaaatggcttataggtttaatggtattggagttacacagaatgt |
| | | tctctatgagaaccaaaaattgattgccaaccaatttaatagtgctattggcaaaattcaagact |
| | | cactttcttccacagcaagtgcacttggaaaacttcaagatgtggtcaaccaaaatgcacaag |
| | | ctttaaacacgcttgttaaacaacttagctccaattttggtgcaatttcaagtgttttaaatgatatc |
| | | ctttcacgtcttgacaaagttgaggctgaagtgcaaattgataggttgatcacaggcagacttc |
| | | aaagtttgcagacatatgtgactcaacaattaattagagctgcagaaatcagagcttctgctaa |
| | | tcttgctgctactaaaatgtcagagtgtgtacttggacaatcaaaaagagttgatttttgtggaa |
| | | agggctatcatcttatgtccttccctcagtcagcacctcatggtgtagtcttcttgcatgtgactt |
| | | atgtccctgcacaagaaaagaacttcacaactgctcctgccatttgtcatgatggaaaagcac |
| | | actttcctcgtgaaggtgtctttgtttcaaatggcacacactggtttgtaacacaaaggaattttt |
| | | atgaaccacaaatcattactacagacaacacatttgtgtctggtaactgtgatgttgtaatagga |
| | | attgtcaacaacacagtttatgatcctttgcaacctgaattagactcattcaaggaggagttaga |
| | | taaatattttaagaatcatacatcaccagatgttgatttaggtgacatctctggcattaatgcttca |
| | | gttgtaaacattcaaaaagaaattgaccgcctcaatgaggttgccaagaatttaaatgaatetc |
| | | tcatcgatctccaagaacttggaaagtatgagcagtatataaaatggccatggtacatttggct |
| | | aggttttatagctggcttgattgccatagtaatggtgacaattatgctttgctgtatgaccagttg |
| | | ctgtagttgtctcaagggctgttgttcttgtggatcctgctgcaaatttgatgaagacgactctg |
| | | agccagtgctcaaaggagtcaaattacattacacataaacgaacttatggattttttatgaga |
| | | atcttcacaattggaactgtaactttgaagcaaggtgaaatcaaggatgctactccttcagattt |
| | | tgttcgogctactgcaacgataccgatacaagcctcactccctttoggatggcttattgttggc |
| | | gttgcacttettgctgtttttcagagegettccaaaatcataaccctcaaaaagagatggcaact |
| | | agcactctccaagggtgttcactttgtttgcaacttgctgttgttgtttgtaacagtttactcacac |
| | | cttttgctegttgctgctggccttgaagccccttttctctatotttatgetttagtctacttcttgcag |
| | | agtataaactttgtaagaataataatgaggctttggctttgctggaaatgccgttecaaaaacc |
| | | cattactttatgatgccaactattttctttgctggcatactaattgttacgactattgtataccttaca |
| | | atagtgtaacttcttcaattgtcattacttcaggtgatggcacaacaagtcctatttctgaacatg |
| | | actaccagattggtggttatactgaaaaatgggaatctggagtaaaagactgtgttgtattaca |
| | | cagttacttcacttcagactattaccagetgtactcaactcaattgagtacagacactggtgttg |
| | | aacatgttaccttcttcatctacaataaaattgttgatgagcctgaagaacatgtccaaattcac |
| | | acaatcgacggttcatccggagttgttaatccagtaatggaaccaatttatgatgaaccgacg |
| | | acgactactagcgtgcctttgtaagcacaagctgatgagtacgaacttatgtactcattegtttc |
| | | ggaagagacaggtacgttaatagttaatagegtacttctttttcttgctttogtggtattcttgcta |
| | | gttacactagccatccttactgcgcttcgattgtgtgcgtactgctgcaatattgttaacgtgagt |
| | | cttgtaaaaccttctttttaegtttactetegtgttaaaaatctgaattettctagagttcctgatcttc |
| | | tggtctaaacgaactaaatattatattagtttttctgtttggaactttaattttagccatggcagatt |
| | | ccaacggtactattaccgttgaagagcttaaaaagctccttgaacaatggaacctagtaatag |
| | | gtttcctattccttacatggatttgtcttctacaatttgcctatgccaacaggaataggtttttgtat |
| | | ataattaagttaattttectetggetgttatggccagtaactttagcttgttttgtgettgetgetgttt |
| | | acagaataaattggatcaccggtggaattgctatcgcaatggcttgtcttgtaggcttgatgtg |
| | | gctcagctacttcattgettetttcagactgtttgcgegtacgegttecatgtggtcatteaatcc |
| | | agaaactaacattcttctcaacgtgccactccatggcactattctgaccagaccgcttctagaa |
| | | agtgaactegtaatcggagctgtgatccttegtggacatcttcgtattgctggacaccatctag |
| | | gacgctgtgacatcaaggacctgcctaaagaaatcactgttgctacatcacgaacgetttott |
| | | attacaaattgggagcttcgcagcgtgtagcaggtgactcaggttttgctgcatacagtcgct |
| | | acaggattggcaactataaattaaacacagaccattccagtagcagtgacaatattgctttgct |
| | | tgtacagtaagtgacaacagatgtttcatctcgttgactttcaggttactatagcagagatattac |
| | | taattattatgaggacttttaaagtttccatttggaatcttgattacatcataaacctcataattaaa |
| | | aatttatctaagtcactaactgagaataaatattctcaattagatgaagagcaaccaatggaga |
| | | ttgattaaacgaacatgaaaattattcttttcttggcactgataacactcgctacttgtgagcttta |
| | | tcactaccaagagtgtgttagaggtacaacagtacttttaaaagaaccttgetettctggaacat |
| | | acgagggcaattcaccatttcatcctctagctgataacaaatttgcactgacttgctttagcact |
| | | caatttgcttttgcttgtcctgacggcgtaaaacacgtctatcagttacgtgccagatcagtttca |
| | | cctaaactgttcatcagacaagaggaagttcaagaactttactctccaatttttcttattgttgcg |
| | | gcaatagtgtttataacactttgcttcacactcaaaagaaagacagaatgattgaactttcatta |
| | | attgacttctatttgtgctttttagcctttctgctattcettgttttaattatgettattatottttggttctc |
| | | acttgaactgcaagatcataatgaaacttgtcacgcctaaacgaacatgaaatttettgttttett |
| | | aggaatcatcacaactgtagctgcatttcaccaagaatgtagtttacagtcatgtactcaacat |
| | | caaccatatgtagttgatgacccgtgtcctattcacttctattctaaatggtatattagagtagga |
| | | gctagaaaatcagcacctttaattgaattgtgcgtggatgaggctggttctaaatcacccattc |
| | | agtacatcgatatcggtaattatacagtttcctgtttaccttttacaattaattgccaggaacctaa |
| | | attgggtagtcttgtagtgcgttgttcgttctatgaagactttttagagtatcatgacgttcgtgtt |
| | | gttttagatttcatctaaacgaacaaactaaaatgtctgataatggaccccaaaatcagegaaa |
| | | tgcaccccgcattacgtttggtggaccctcagattcaactggcagtaaccagaatggagaac |
| | | gcagtggggcgegatcaaaacaacgteggccccaaggtttacccaataatactgegtettg |
| | | gttcaccgctctcactcaacatggcaaggaagaccttaaattccctogaggacaaggogttc |
| | | caattaacaccaatagcagtccagatgaccaaattggctactaccgaagagctaccagacg |
| | | aattcgtggtggtgacggtaasatgaaagatctcagtccaagatggtatttctactacctagga |
| | | actgggccagaagetggacttcectafggtgctaacaaagacegcatcatagggttgcaac |
| | | tgagggagcottgaatacaccaaaagatcacattggcaccegcaatcctgctaacaatgetg |
| | | caategtgctacaacttoctcaaggaacaacattgccasaaggcttctacgcagaagggag |
| | | cagaggeggcagtcaagcctettctegftcctcatcacgtagtegcascagttcaagaaattc |
| | | aactccaggcagcagtaggggaacttctectgctagaatggctggcaatggeggtgatget |
| | | gctettgctttgctgotgottgacagattgaaccagettgagagcaaaatgtctggtaaaggcc |
| | | aacaacaacaaggccaaactgteactaagaaatctgctgctgaggcttetaagaagcctcg |
| | | gcaaaaacgtactgccactaaagcatacaatgtaacacaagottteggcagacgtggtcca |
| | | gaacaaacccaaggaaattttggggaccaggaactaatcagacaaggaactgattacaaac |
| | | attggcegcasattgcacaatttgcccccagegcttcagogttctteggaatgtegogcattg |
| | | gcalggaagtcacacettegggaacgtggttgacetacacaggtgccatcaaattggatgac |
| | | aaagatccaaatttcaaagatcaagtcattttgetgaataagcatattgacgcatacaaaacatt |
| | | cccaccaacagagcctaaaaaggacaaaaagaagaaggetgatgaaactcaagcettace |
| | | gcagagacagaagaaacagcaaactgtgactcttcttcetgctgcagatttggatgatttotec |
| | | aaacaattgcascaatccatgagcagtgctgactcaactcaggectaaactcatgcagacca |
| | | cacaaggcagatgggctatataaacgttttegettttcogtttacgatatatagtctactcttgtg |
| | | cagaatgaattctegtaactacatagcacaagtagatgtagttaactttaatctcacatagcaat |
| | | ctttaatcagtgtgtaacattagggaggacttgaaagagccaccacattttcacegaggecac |
| | | goggagtacgalcgagtgtacagtgaacaatgctagggagagctgcctatatggaagage |
| | | cctaatgtgtaaaattaattttagtagtgctatccccatgtgattttaatagcttcttaggagaatg |
| | | acaaaaaaaa |

⁺ln = Locked nucleic acid (LNA) = 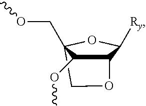
wherein R_{y} is a nucleobase;
lnA = Locked nucleic acid (LNA) A;
ln(5m)C = In(5m)C =Locked nucleic acid (LNA)-5 methyl C;
lnG = Locked nucleic acid (LNA) G;
lnT = Locked nucleic acid (LNA) T;
(5m)C = 5 methyl C;
scp = spirocyclopropyl;
sep(5m)C = cyclopropyl-5 methyl C;
(5OH)C = 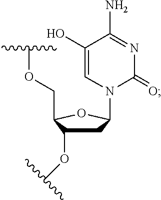 [SMILES: *OC[C@H]1O[C@@H](n2cc(O)c(N)nc2=O)C[C@H]1O*]
po = phosphodiester linkage;
ps = phosphorothioate linkage

What is claimed is:

1. An oligonucleotide comprising a nucleotide comprising a structure selected from: 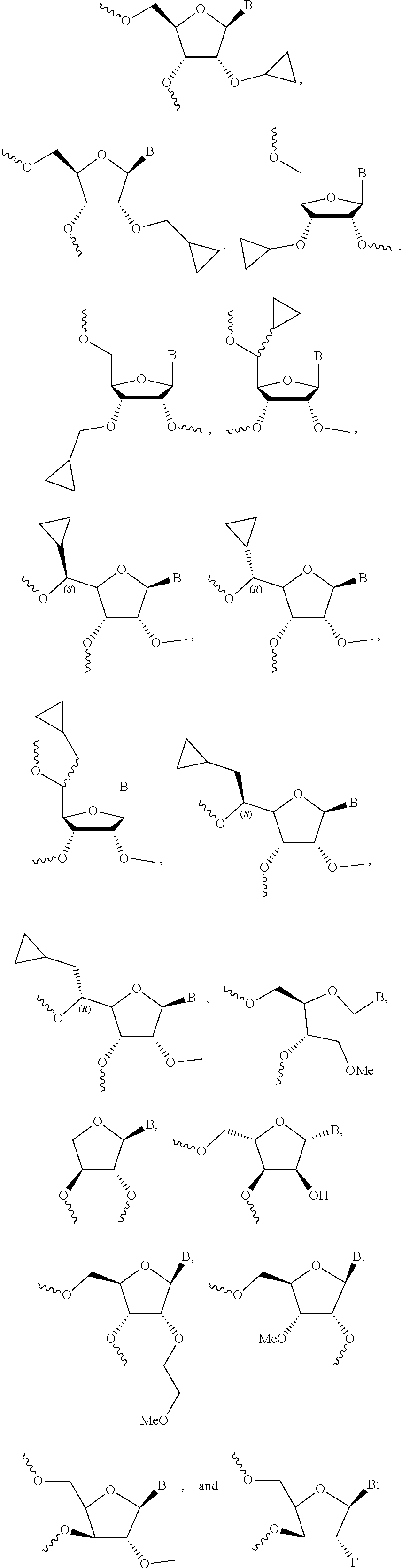
wherein B is a nucleobase, aryl, heteroaryl, or H, and wherein [symbol] represents a phosphodiester linkage, a phosphorothioate linkage, a mesyl phosphoroamidate linkage, or H.

2. The oligonucleotide of claim 1, wherein B is selected from adenine, guanine, cytosine, thymine, and uracil.

3. The oligonucleotide of claim 1, wherein the nucleotide comprises a structure selected from: 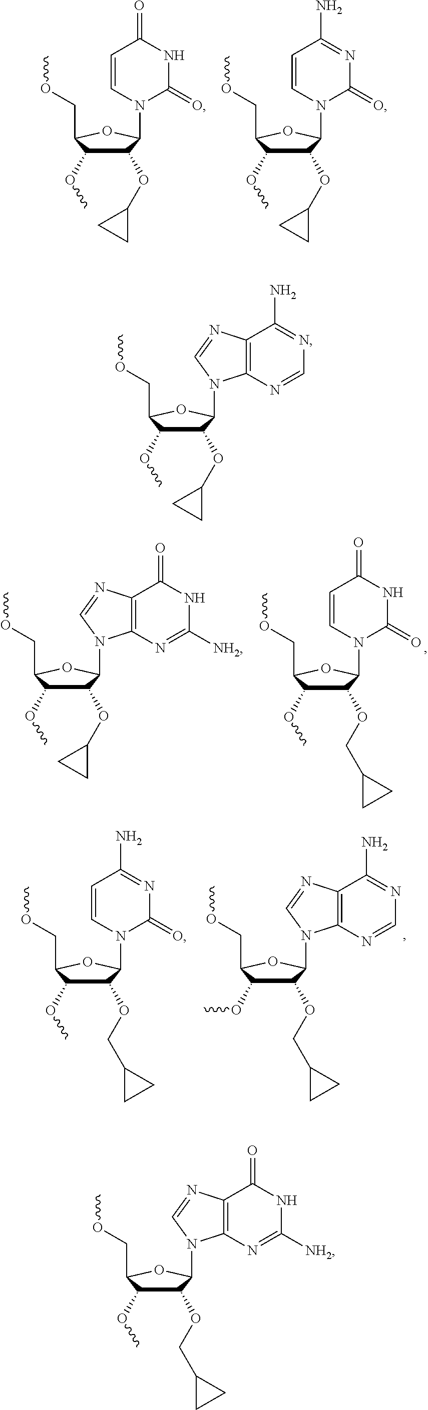 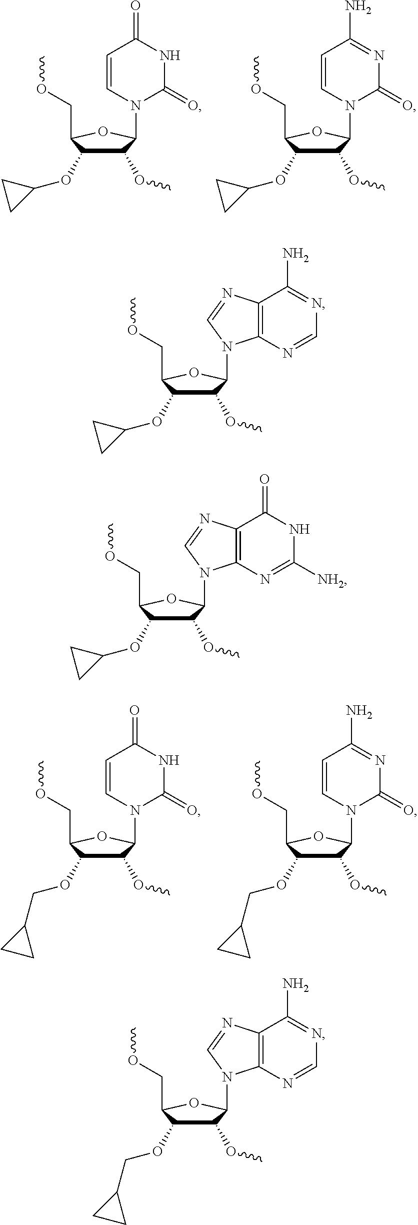 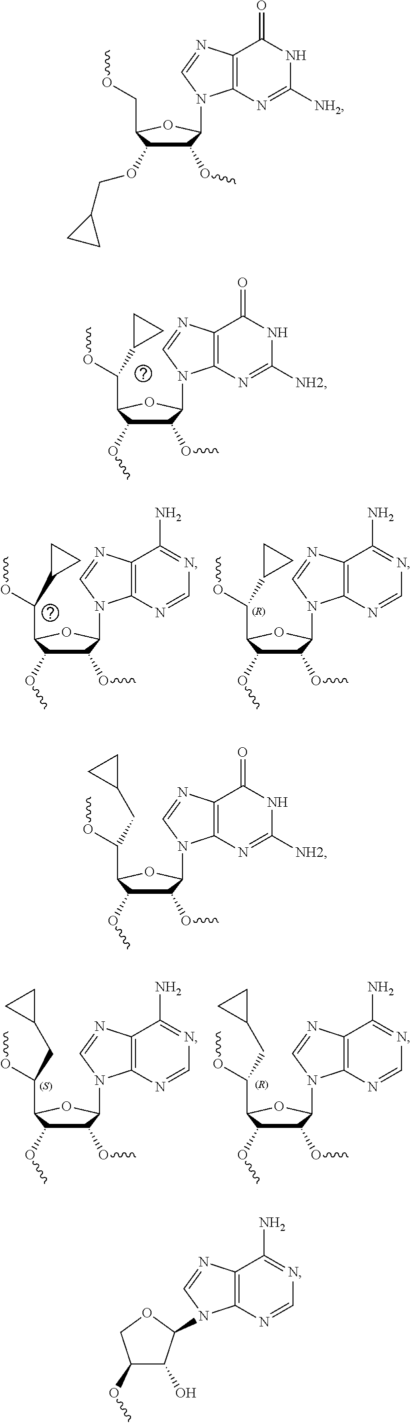 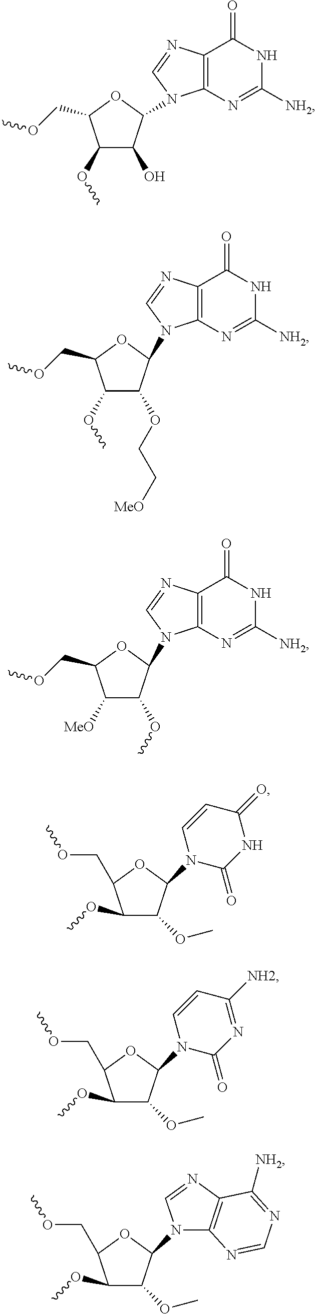 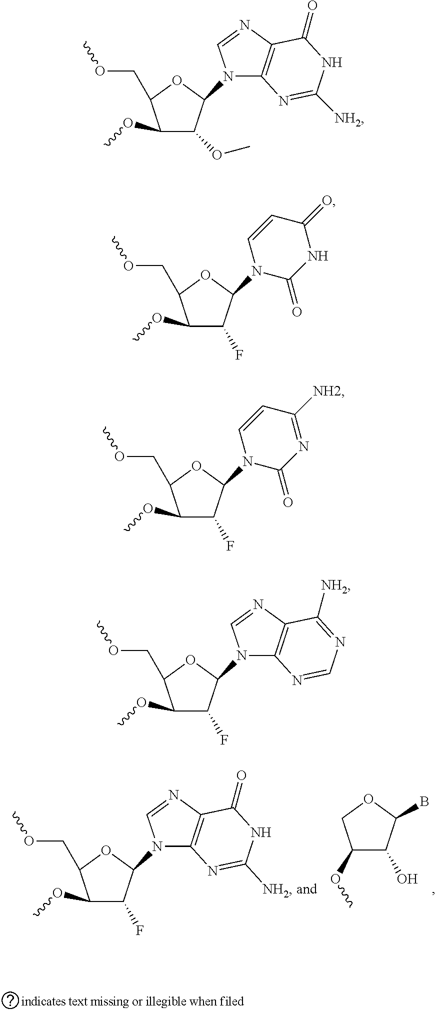
wherein B is a nucleobase, aryl, heteroaryl, or H, and wherein [symbol] represents a phosphodiester linkage, a phosphorothioate linkage, a mesyl phosphoroamidate linkage, or H.

4. The oligonucleotide of any one of claims 1-3, wherein the oligonucleotide comprises at least 2, at least 3, at least 4, or at least 5 nucleotides comprising a structure independently selected from: 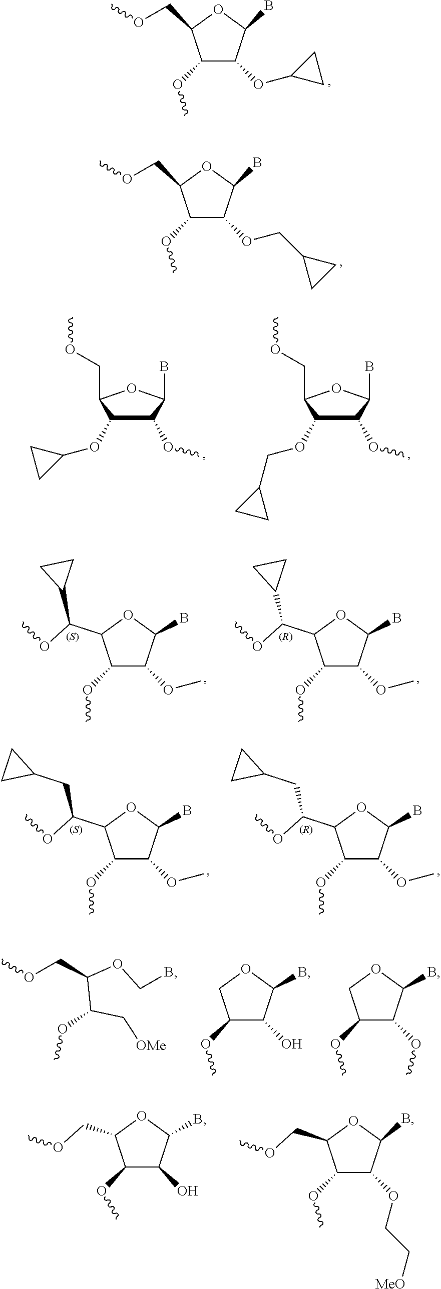 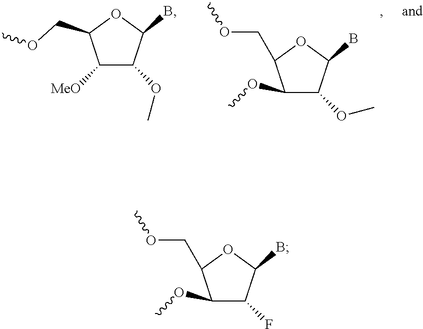
wherein B is a nucleobase, aryl, heteroaryl, or H, and wherein [symbol] represents a phosphodiester linkage, a phosphorothioate linkage, a mesyl phosphoroamidate linkage, or H.

5. An oligonucleotide comprising a nucleotide analog comprising a structure of: 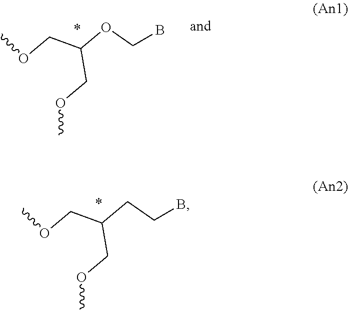
wherein B is a nucleobase, an aryl, heteroaryl, or H; wherein [symbol] represents a phosphodiester linkage, a phosphorothioate linkage, or H; and wherein * represent chiral center.

6. The nucleotide analog of claim 5, wherein B is selected from adenine, guanine, cytosine, thymine, and uracil.

7. The oligonucleotide of claim 5 or 6, wherein the oligonucleotide comprises at least 2, at least 3, at least 4, or at least 5 nucleotide analogs comprising a structure independently selected from: 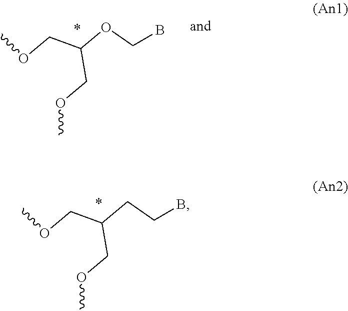
wherein B is a nucleobase, an aryl, heteroaryl, or H; wherein [symbol] represents a phosphodiester linkage, a phosphorothioate linkage, or H; and wherein * represent chiral center.

8. An oligonucleotide comprising a nucleotide comprising a structure selected from: 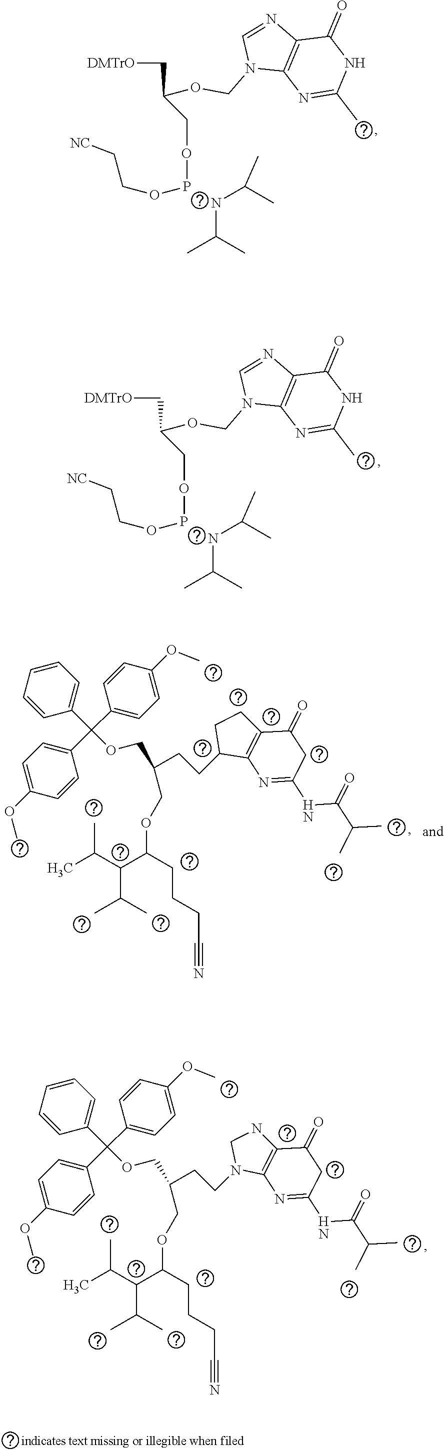
wherein B is a nucleobase, aryl, heteroaryl, or H; wherein [symbol] represents a phosphodiester linkage, a phosphorothioate linkage, or a mesyl phosphoroamidate linkage.

9. An oligonucleotide comprising a structure of: 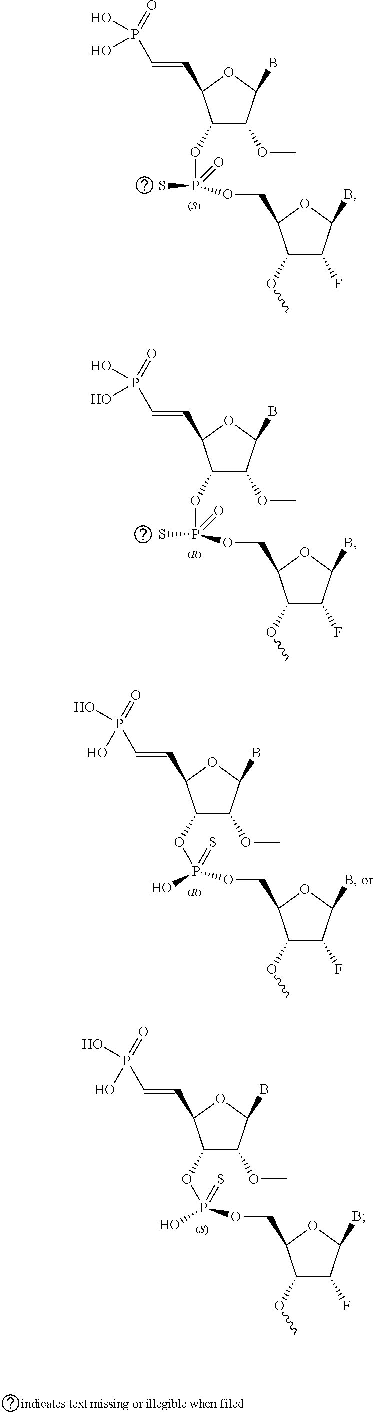
wherein each B is independently selected from a nucleobase, aryl, heteroaryl, and H; wherein [symbol] represents a phosphodiester linkage, a phosphorothioate linkage, or a mesyl phosphoroamidate linkage.

10. The oligonucleotide of any one of claims 1-9, wherein the oligonucleotide is selected from a short interfering nucleic acid (siNA), an antisense oligonucleotide (ASO), a steric blocker, a short hairpin RNA (shRNA), and an mRNA.

11. A short interfering nucleic acid (siNA), comprising a sense strand and an antisense strand, wherein the sense strand, the antisense strand, or both comprise at least 1, at least 2, at least 3, at least 4, or at least 5 nucleotide(s) independently selected from: 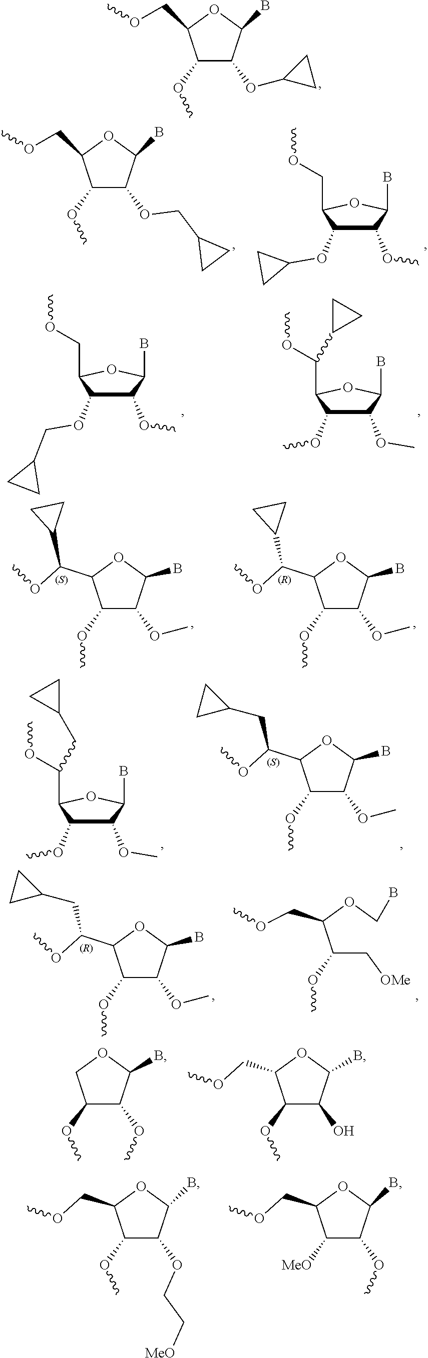 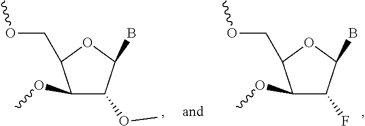
or at least 1, at least 2, at least 3, at least 4, or at least 5 nucleotide analog(s) independently selected from: 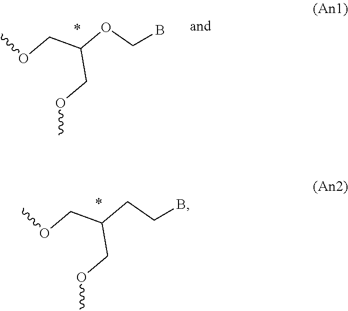
wherein B is a nucleobase, an aryl, heteroaryl, or H; wherein [symbol] represents a phosphodiester linkage, a phosphorothioate linkage, or H; and wherein * represent chiral center.

11. A short interfering nucleic acid (siNA) comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(ix) is 15 to 30 nucleotides in length; and
(x) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(xi) is 15 to 30 nucleotides in length; and
(xii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 7, 8, 10, 14, 16, 17, and/or 18 from the 5′ end of the second nucleotide sequence is a 2′-fluoro nucleotide;
wherein the sense strand and/or the antisense strand comprise at least 1, at least 2, at least 3, at least 4, or at least 5 nucleotide(s) selected from: 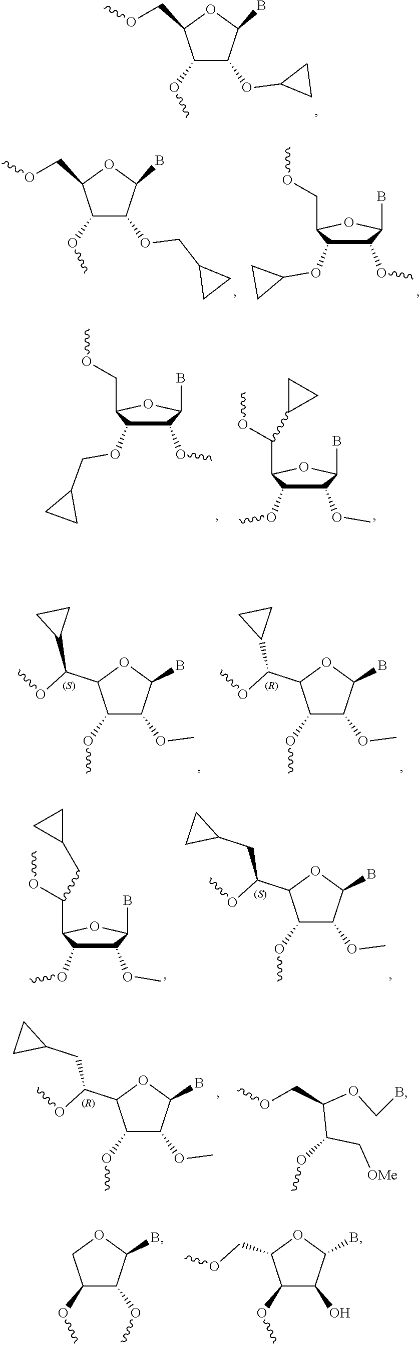 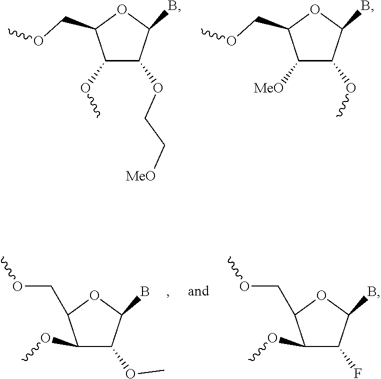
or at least 1, at least 2, at least 3, at least 4, or at least 5 nucleotide analog(s) selected from: 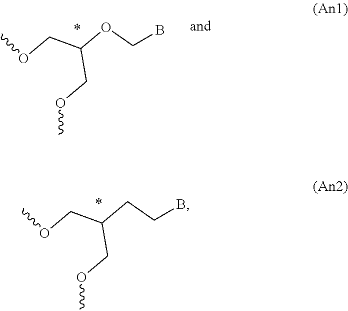
wherein B is a nucleobase, an aryl, heteroaryl, or H; wherein [symbol] represents a phosphodiester linkage, a phosphorothioate linkage, or H; and wherein * represent chiral center.

12. The siNA according to claim 10 or 11, wherein the antisense strand comprises a 5′-stabilized end cap selected from: 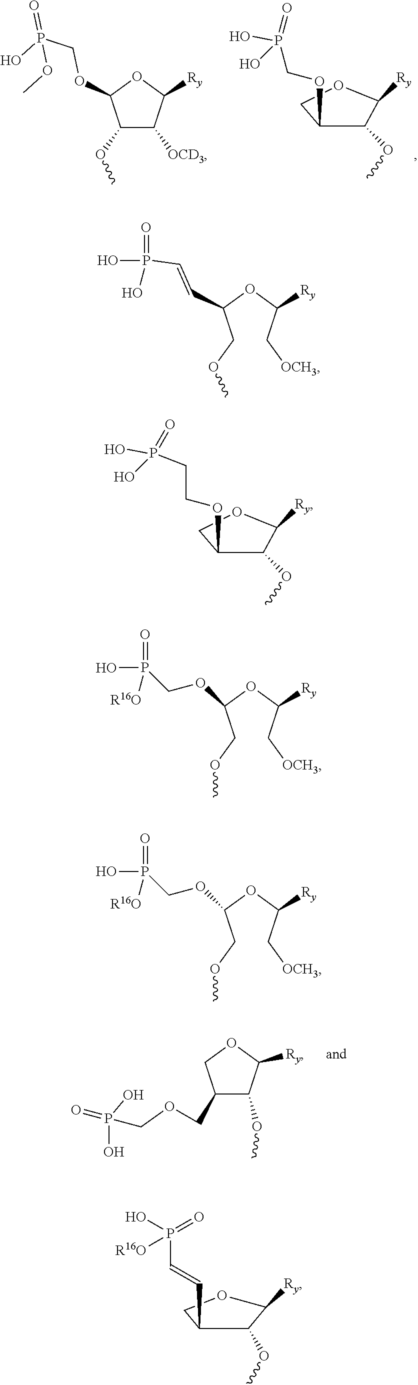
wherein R_{y} is a nucleobase and R¹⁵ is H or CH₃, and wherein [symbol] represents a phosphodiester linkage, a phosphorothioate linkage, or a mesyl phosphoroamidate linkage.

13. The siNA according to claim 10 or 11, wherein the antisense strand comprises a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61), Formula (62), and Formula (63), wherein Rₓ is a nucleobase, aryl, heteroaryl, or H.

14. The siNA according to claim 10 or 11, wherein the antisense strand comprises a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′, wherein Rₓ is a nucleobase, aryl, heteroaryl, or H.

15. The siNA according to claim 10 or 11, wherein the antisense strand comprises a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY).

16. The siNA according to claim 10 or 11, wherein the antisense strand comprises a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY).

17. The siNA according to claim 10 or 11, wherein the antisense strand comprises a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′).

18. A short interfering nucleic acid (siNA), comprising a sense strand and an antisense strand, wherein the antisense strand comprises a 5′vinyl phosphonate moiety comprising a structure of: 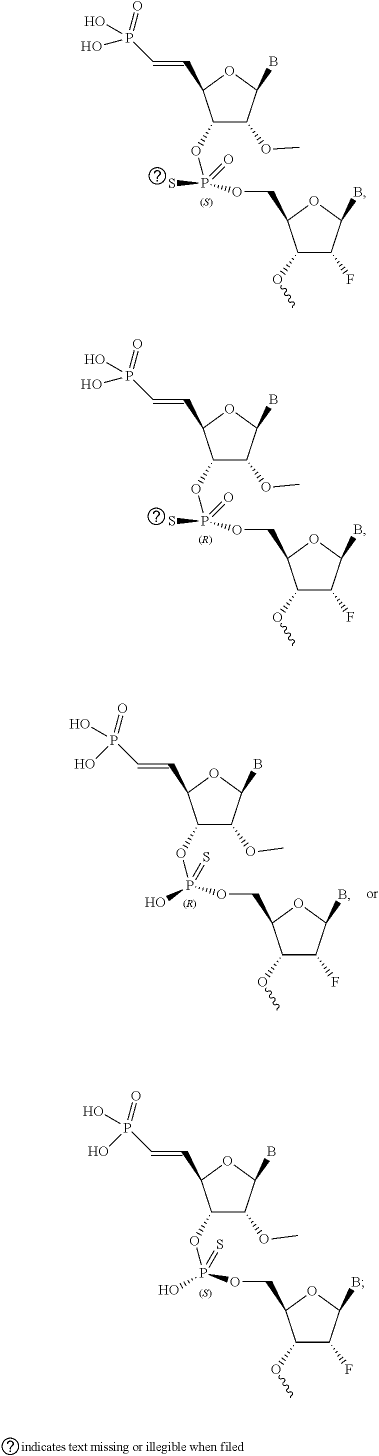
wherein each B is independently selected from a nucleobase, aryl, heteroaryl, and H;
wherein [symbol] represents a phosphodiester linkage, a phosphorothioate linkage, or a mesyl phosphoroamidate linkage.

19. The siNA of claim 18, wherein the structure is:

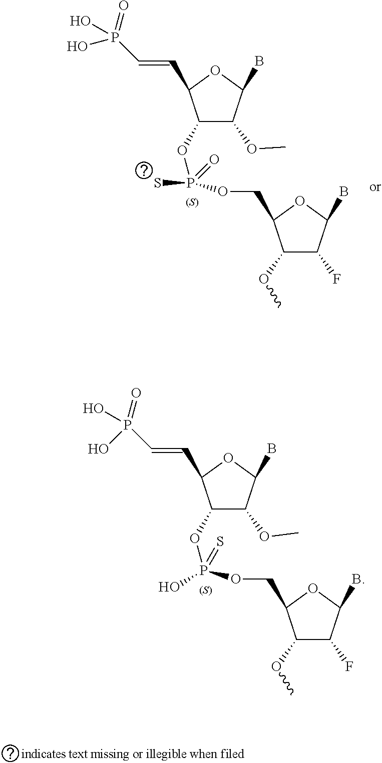

20. The siNA of claim 18, wherein the structure is:

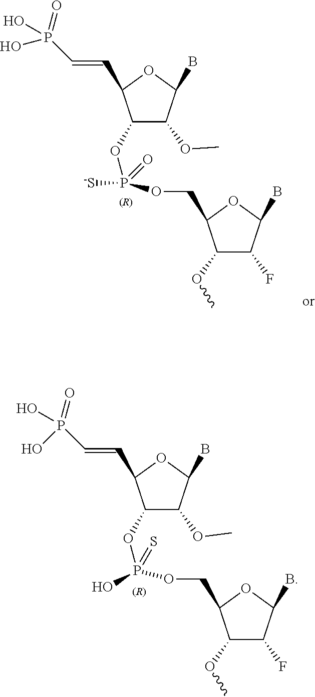

21. The siNA of any one of claims 18-20, wherein the sense strand, the antisense strand, or both comprise at least 1, at least 2, at least 3, at least 4, or at least 5 nucleotide(s) comprising a structure independently selected from: 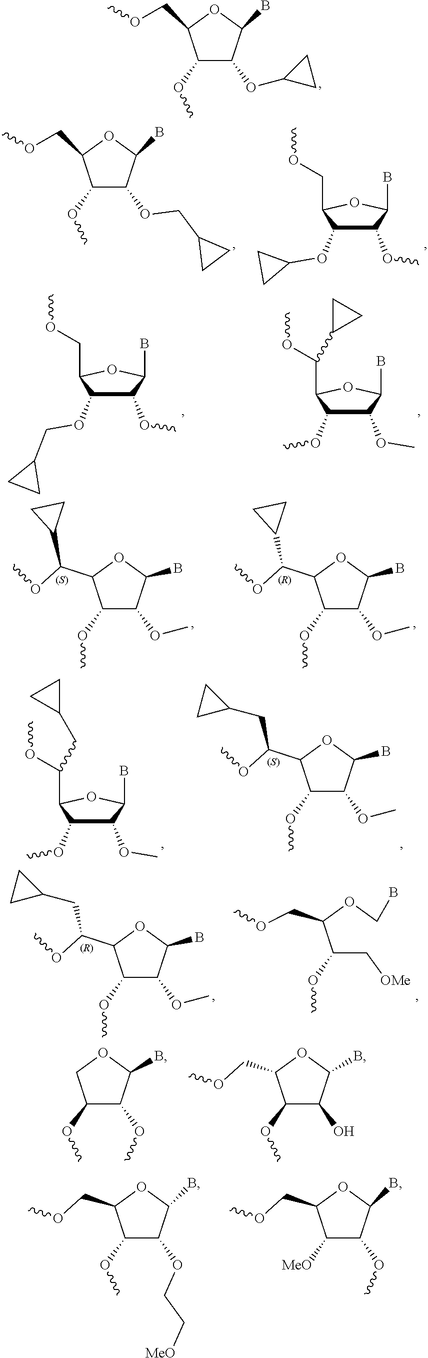 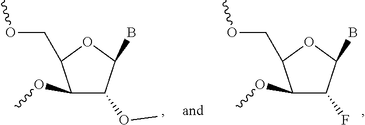
or at least 1, at least 2, at least 3, at least 4, or at least 5 nucleotide analog(s) comprising a structure independently selected from: 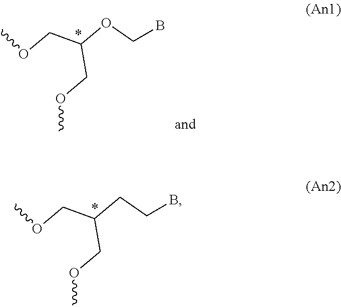
wherein B is a nucleobase, an aryl, heteroaryl, or H; wherein [symbol] represents a phosphodiester linkage, a phosphorothioate linkage, or H; and wherein * represent chiral center.

22. The siNA of any one of claims 10-21, wherein the sense strand, the antisense strand, or both each independently comprise 1 or more phosphorothioate internucleoside linkages.

23. The siNA of any one of claims 10-22, wherein the siNA further comprises a phosphorylation blocker.

24. The siNA molecule according to any one of claims 10-23, wherein the sense strand comprises at least 1, 2, 3, 4, S, 6, 7, 8, 9, 10, 11, 12, 13, 14, 15 or more phosphorothioate internucleoside linkages

25. The siNA molecule of claim 24, wherein:
(i) at least one phosphorothioate internucleoside linkage in the sense strand is between the nucleotides at positions 1 and 2 from the 5′ end of the sense strand; and/or
(ii) at least one phosphorothioate internucleoside linkage in the sense strand is between the nucleotides at positions 2 and 3 from the 5′ end of the sense strand.

26. The siNA molecule according to any one of claims 10-25, wherein the antisense strand further comprises at least 1, 2, 3, 4, 5, 6, 7, 8, 9, 10, 11, 12, 13, 14, 15 or more phosphorothioate internucleoside linkages.

27. The siNA molecule of claim 26, wherein:
(i) at least one phosphorothioate internucleoside linkage in the antisense strand is between the nucleotides at positions 1 and 2 from the 5′ end of the antisense strand;
(ii) at least one phosphorothioate internucleoside linkage in the antisense strand is between the nucleotides at positions 2 and 3 from the 5′ end of the antisense strand;
(iii) at least one phosphorothioate internucleoside linkage in the antisense strand is between the nucleotides at positions 1 and 2 from the 3′ end of the secantisense strand; and/or
(iv) at least one phosphorothioate internucleoside linkage is between the nucleotides at positions 2 and 3 from the 3′ end of the antisense strand.

28. The siNA molecule according to any one of claims 10-27, wherein the sense strand comprises at least 1, 2, 3, 4, 5, 6, 7, 8, 9, 10, 11, 12, 13, 14, 15 or more mesyl phosphoroamidate internucleoside linkages.

29. The siNA molecule of claim 28, wherein:
(i) at least one mesyl phosphoroamidate internucleoside linkage in the sense strand is between the nucleotides at positions 1 and 2 from the 5′ end of the sense strand; and/or
(ii) at least one mesyl phosphoroamidate internucleoside linkage is between the nucleotides at positions 2 and 3 from the 5′ end of the sense strand.

30. The siNA molecule according to any one of claims 10-29, wherein the antisense strand further comprises at least 1, 2, 3, 4, 5, 6, 7, 8, 9, 10, 11, 12, 13, 14, 15 or more mesyl phosphoroamidate internucleoside linkages.

31. The siNA molecule of claim 30, wherein:
(i) at least one mesyl phosphoroamidate internucleoside linkage in the antisense strand is between the nucleotides at positions 1 and 2 from the 5′ end of the antisense strand;
(ii) at least one mesyl phosphoroamidate internucleoside linkage in the antisense strand is between the nucleotides at positions 2 and 3 from the 5′ end of the antisense strand;
(iii) at least one mesyl phosphoroamidate internucleoside linkage in the antisense strand is between the nucleotides at positions 1 and 2 from the 3′ end of the antisense strand; and/or
(iv) at least one mesyl phosphoroamidate internucleoside linkage is between the nucleotides at positions 2 and 3 from the 3′ end of the antisense strand.

32. The siNA molecule according to any one of claims 10-31, wherein the sense strand, the antisense strand, or both each independently comprise at least 1, at least 2, at least 3, at least 4, or at least 5 or more of 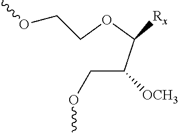
wherein Rₓ is a nucleobase, aryl, heteroaryl, or H), 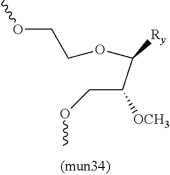
wherein R^{y} is a nucleobase, 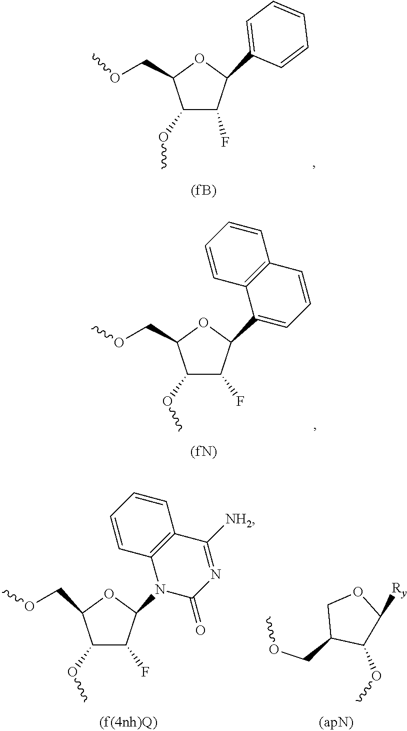
wherein R_{y} is a nucleobase, or combinations thereof.

33. The siNA according to any one of claims 10-32, wherein the siNA further comprises a galactosamine.

34. The siNA according to claim 33, wherein the galactosamine is N-acetylgalactosamine (GalNAc) of Formula (VI): 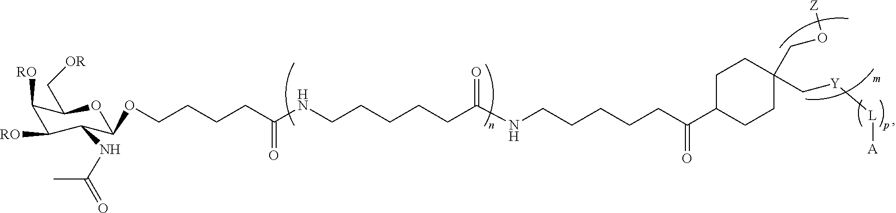
wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

35. The siNA according to claim 33, wherein the galactosamine is N-acetylgalactosamine (GalNAc) of Formula (VII): 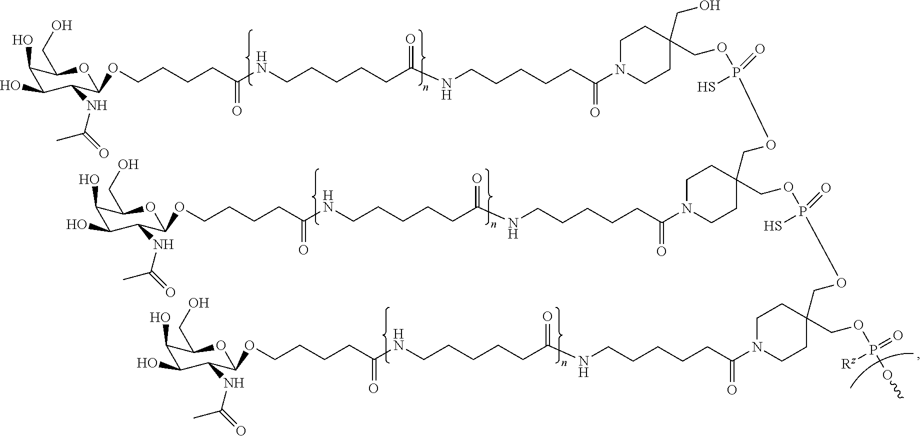
wherein R^{z} is OH or SH; and each n is independently 1 or 2.

36. The siNA according to any one of claims 10-35, wherein:
(i) at least one end of the siNA is a blunt end;
(ii) at least one end of the siNA comprises an overhang, wherein the overhang comprises at least one nucleotide; or
(iii) both ends of the siNA comprise an overhang, wherein the overhang comprises at least one nucleotide.

37. The siNA according to any one of claims 10-36, wherein;
(i) the target gene is a viral gene;
(ii) the target gene is a gene is from a DNA virus.
(iii) the target gene is a gene from a double-stranded DNA (dsDNA) virus;
(iv) the target gene is a gene from a hepadnavirus;
(v) the target gene is a gene from a a hepatitis B virus (HBV);
(vi) the target gene is a gene from a HBV of any one of genotypes A-J; or
(vii) the target gene is selected from the S gene or X gene of a HBV

38. An siNA as shown in Table 1, Table 2, Table 3, Table 4, Table 5, Table 6, Table 7, Table 8, Table 9, or Table 10, Table 11, Table 12, Table 13, Table 14, Table 15, Table 16, Table 17, or Table 18.

39. A composition comprising the siNA according to any one of claims 10-38; and a pharmaceutically acceptable excipient.

40. The composition of claim 39 further comprising 2, 3, 4, 5, 6, 7, 8, 9, 10 or more siNAs according to any one of claims 10-38.

41. The composition according to claim 39 or 40 further comprising an additional treatment agent.

42. The composition of claim 41, wherein the additional treatment agent is selected from a nucleotide analog, nucleoside analog, a capsid assembly modulator (CAM), a recombinant interferon, an entry inhibitor, a small molecule immunomodulatory, and oligonucleotide therapy.

43. The composition of claim 42, wherein the oligonucleotide therapy is an additional siNA, an antisense oligonucleotide (ASO), NAPs, or STOPS™.

44. A method of treating a disease in a subject in need thereof, comprising administering to the subject the siNA according to any one of claims 10-38 or a composition according to any one of claims 39-43.

45. The method of claim 44, wherein the disease is a viral disease, which is optionally caused by a DNA virus or a double stranded DNA (dsDNA) virus.

46. The method of claim 45, wherein the dsDNA virus is a hepadnavirus.

47. The method of claim 46, wherein the hepadnavirus is a hepatitis B virus (HBV), and optionally wherein the HBV is selected from HBV genotypes A-J.

48. The method of claim 47 further comprising administering an additional HBV treatment agent.

49. The method of claim 48, wherein the siNA or the composition and the additional HBV treatment agent are administered concurrently or administered sequentially.

50. The method of claim 48 or 49, wherein the additional HBV treatment agent is selected from a nucleotide analog, nucleoside analog, a capsid assembly modulator (CAM), a recombinant interferon, an entry inhibitor, a small molecule immunomodulator and oligonucleotide therapy.

51. The method of claim 45, wherein the viral disease is a disease caused by a coronavirus, and optionally wherein the coronavirus is SARS-COV-2.

52. The method of claim 44, wherein the disease is a liver disease.

53. The method of claim 52, wherein the liver disease is a nonalcoholic fatty liver disease (NAFLD) or hepatocellular carcinoma (HCC).

54. The method of claim 53, wherein the NAFLD is nonalcoholic steatohepatitis (NASH).

55. The method of any one of claims 52-54 further comprising administering to the subject a liver disease treatment agent.

56. The method of claim 55, wherein the liver disease treatment agent is selected from a peroxisome proliferator-activator receptor (PPAR) agonist, farnesoid X receptor (FXR) agonist, lipid-altering agent, and incretin-based therapy.

57. The method of claim 56, wherein (i) the PPAR agonist is selected from a PPARα agonist, dual PPARα/δ agonist, PPARγ agonist, and dual PPARα/γ agonist; (ii) the lipid-altering agent is aramchol; or (iii) the incretin-based therapy is a glucagon-like peptide 1 (GLP-1) receptor agonist or dipeptidyl peptidase 4 (DPP-4) inhibitor.

58. The method of any one of claims 55-57, wherein the siNA or composition and the liver disease treatment agent are administered concurrently or administered sequentially.

59. The method of any of one claims 44-58, wherein the siNA or the composition is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg.

60. The method of any of one claims 44-58, wherein the siNA or the composition is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

61. The method of any of one claims 44-60, wherein the siNA or the composition is administered at least 1, 2, 3, 4, 5, 6, 7, 8, 9, or 10 times.

62. The method of any of one claims 44-61, wherein the siNA or the composition is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month.

63. The method of any of one claims 44-61, wherein the siNA or the composition is administered at least once every 1, 2, 3, 4, 5, 6, 7, 8, 9, 10, 11, 12, 13, 14, 15, 16, 17, 18, 19, 20, or 21 days.

64. The method of any of one claims 44-61, wherein the siNA or the composition is administered for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

65. The method of any of one claims 44-64, wherein the siNA or the composition is administered at a single dose of 5 mg/kg or 10 mg/kg, at three doses of 10 mg/kg once a week, at three doses of 10 mg/kg once every three days, or at five doses of 10 mg/kg once every three days.

66. The method of any of one claims 44-64, wherein the siNA or the composition is administered at six doses of ranging from 1 mg/kg to 15 mg/kg, 1 mg/kg to 10 mg/kg, 2 mg/kg to 15 mg/kg, 2 mg/kg to 10 mg/kg, 3 mg/kg to 15 mg/kg, or 3 mg/kg to 10 mg/kg; wherein the first dose and second dose are optionally administered at least 3 days apart; wherein the second dose and third dose are optionally administered at least 4 days apart; and wherein the third dose and fourth dose, fourth dose and fifth dose, and or fifth dose and sixth dose are optionally administered at least 7 days apart.

67. The method of any one of claims 44-66, wherein the siNA or the composition are administered in a particle or viral vector, wherein the viral vector is optionally selected from a vector of adenovirus, adeno-associated virus (AAV), alphavirus, flavivirus, herpes simplex virus, lentivirus, measles virus, picornavirus, poxvirus, retrovirus, and rhabdovirus.

68. The method of claim 67, wherein the viral vector is a recombinant viral vector.

69. The method of claim 67 or 68, wherein the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

70. The method of any one of claims 44-69, wherein the siNA or the composition is administered systemically or administered locally.

71. The method of any one of claims 44-70, wherein the siNA or the composition is administered intravenously, subcutaneously, or intramuscularly.

72. Use of the siNA according to any one of claims 10-38 or the composition according to any one of claims 39-43 for treating a disease in a subject.

73. The use according to claim 72, wherein the disease is a viral disease, which is optionally caused by a DNA virus or a double stranded DNA (dsDNA) virus or the disease.

74. The use according to claim 72, wherein the disease is a liver disease, which is optionally selected from a nonalcoholic fatty liver disease (NAFLD) and hepatocellular carcinoma (HCC).

75. The siNA according to any one of claims 10-38 or the composition according to any one of claims 39-43 for use in treating a disease in a subject.

76. The siNA or composition according to claim 75, wherein the disease is a viral disease, which is optionally caused by a DNA virus or a double stranded DNA (dsDNA) virus or the disease.

77. The siNA or composition according to claim 75, wherein the disease is a liver disease, which is optionally selected from a nonalcoholic fatty liver disease (NAFLD) and hepatocellular carcinoma (HCC).

78. A short interfering nucleic acid (siNA), comprising a sense strand and an antisense strand, wherein the antisense strand comprises a 3′ overhang comprising at least one modified nucleotide selected from 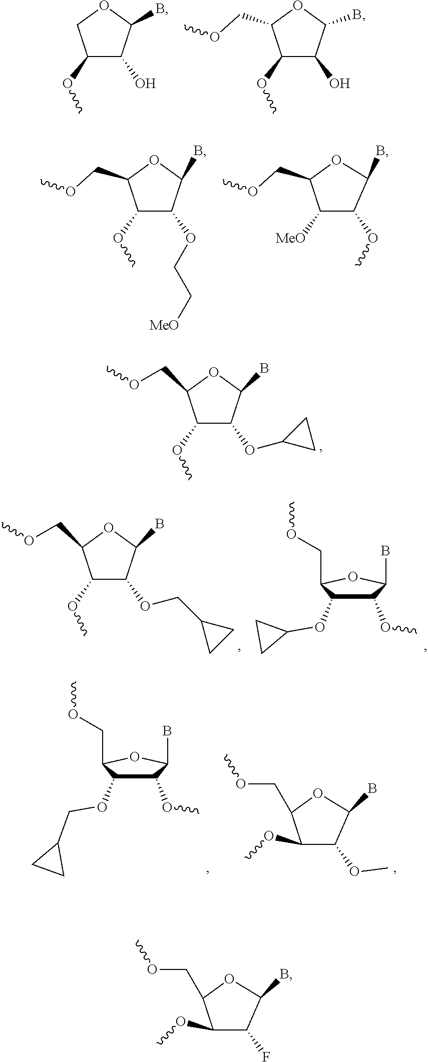
wherein B is a nucleobase, aryl, heteroaryl, or H, and wherein [symbol] represents a phosphodiester linkage, a phosphorothioate linkage, a mesyl phosphoroamidate linkage, or H.

79. The siNA of claim 78, wherein the modified nucleotide is selected from:

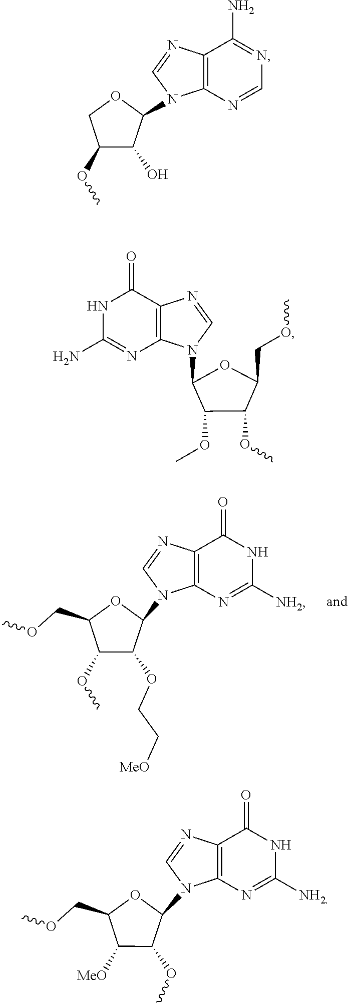

80. The siNA of claim 78 or 79, wherein the modified nucleotide is the last nucleotide or second to last nucleotide at the 3′ end of the antisense strand.

81. The siNA of any one of claims 78-80, wherein the siNA is resistant to nuclease activity relative to a siNA of the same sequence without the modified nucleotide in the 3′ overhang.

82. A phosphoramidite comprising a structure of: 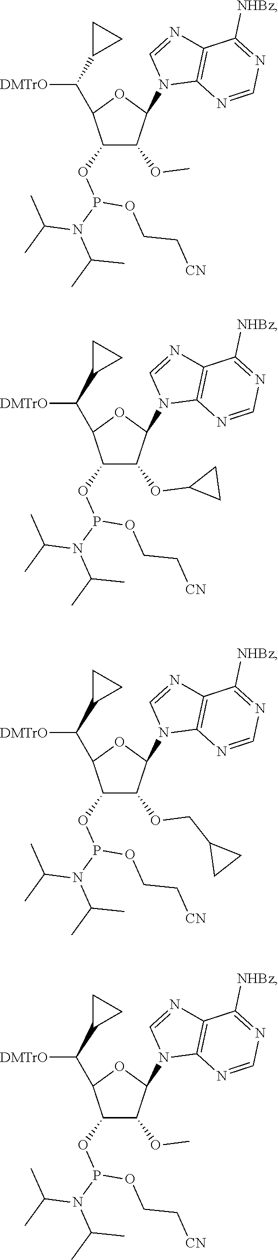 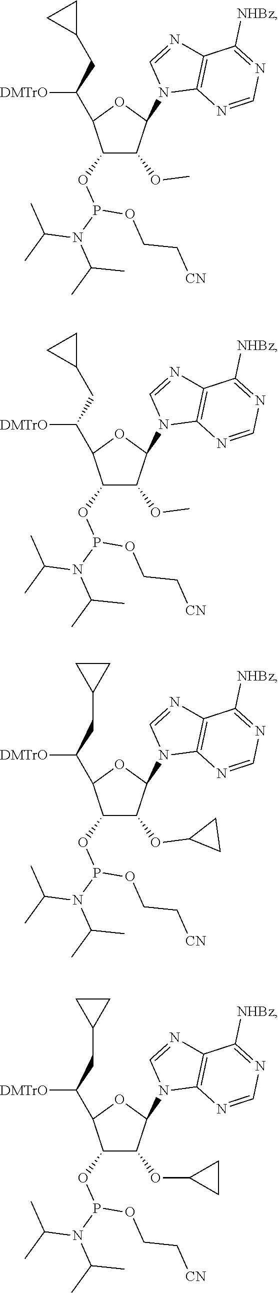 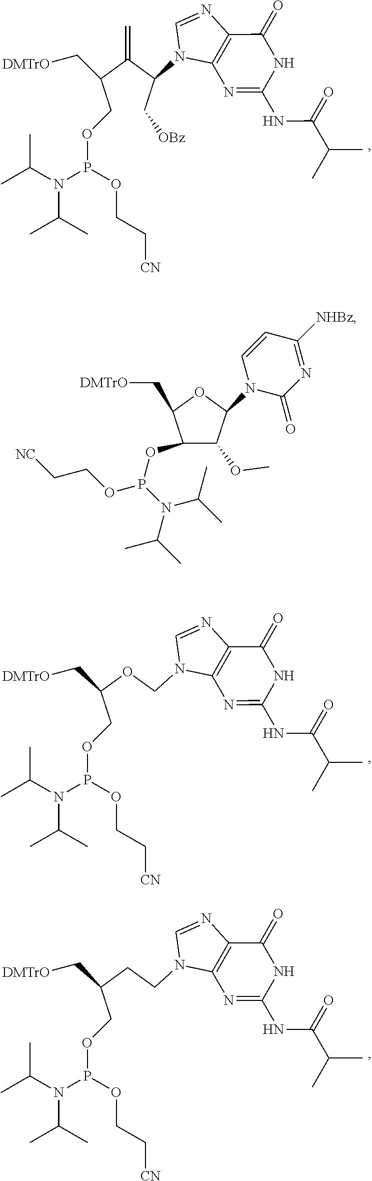 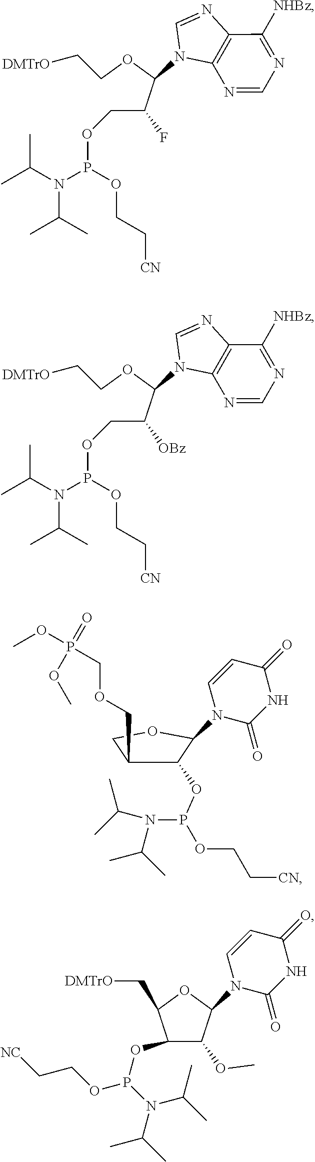 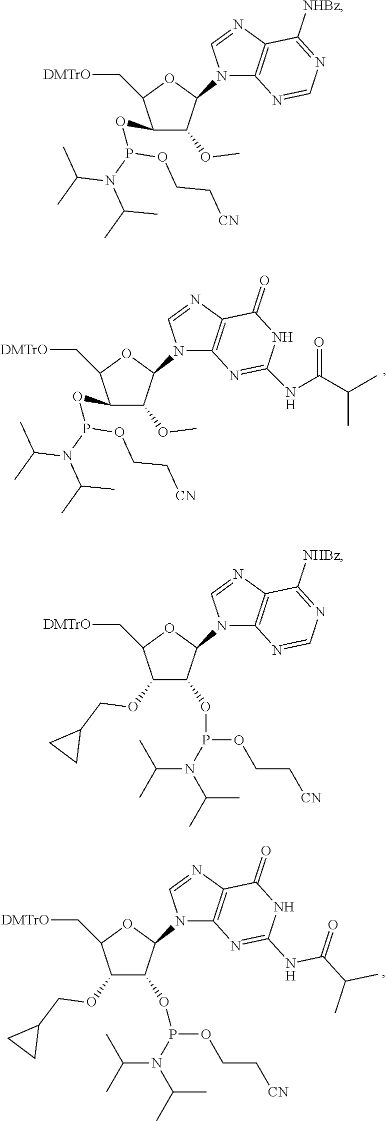 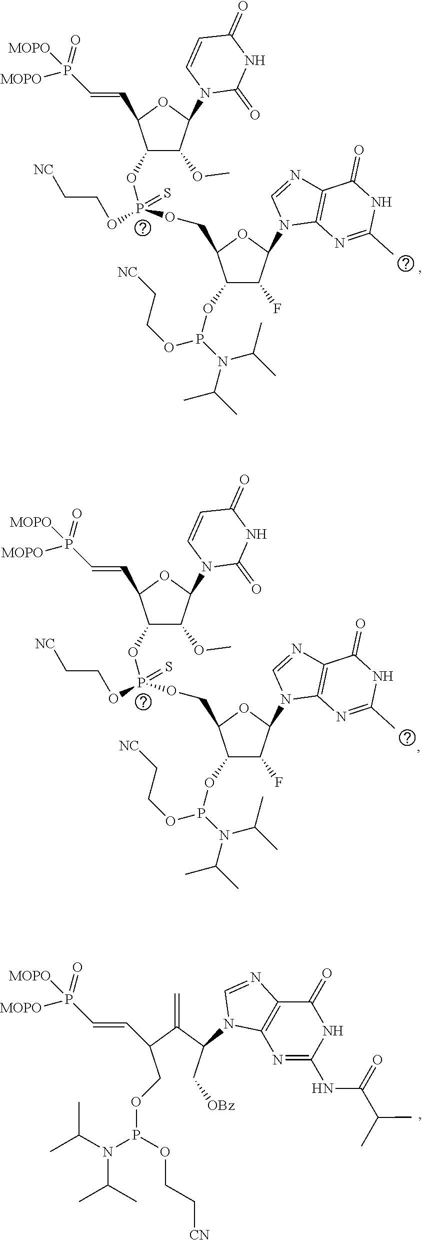 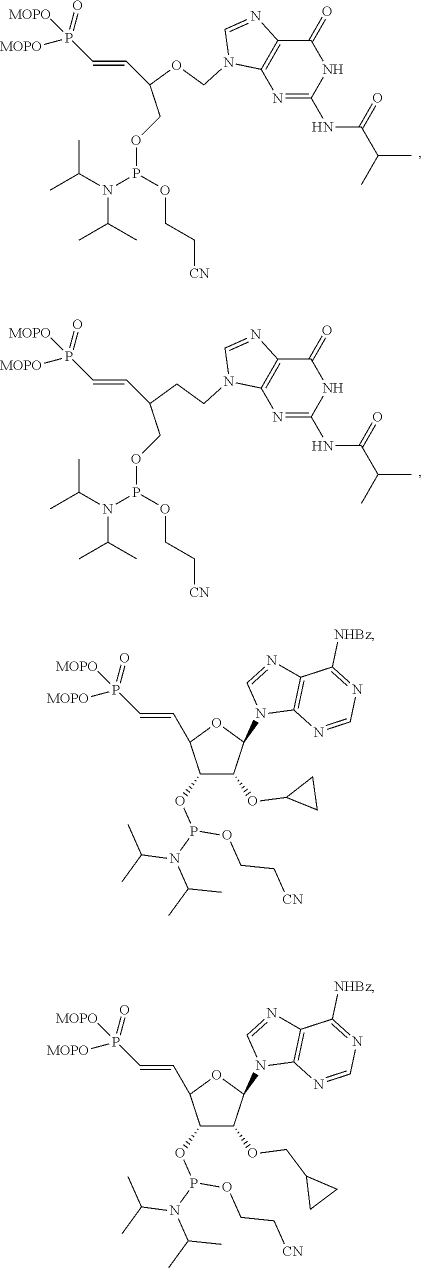 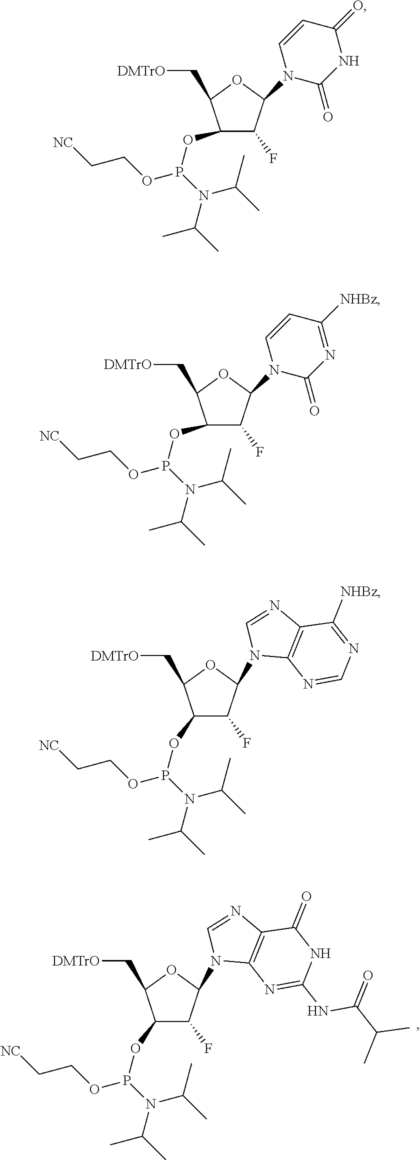 [chemical structure]


SEQUENCE LISTING (166 sequences)

SEQ ID NO: 1 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 2 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = O-methoxyethyl modified nucleotide with phosphorothioate linkage
  modified_base 21: mod_base = OTHER; note = 2'-O-methyl modified nucleotide
attgagagaagtccaccacga

SEQ ID NO: 3 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = Locked nucleic acid with phosphorothioate linkage
  modified_base 21: mod_base = OTHER; note = 2'-O-methyl modified nucleotide
attgagagaagtccaccacga

SEQ ID NO: 4 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = Spirocyclopropane crosslinked nucleotide with phosphorothioate linkage
  modified_base 21: mod_base = OTHER; note = 2'-O-methyl modified nucleotide
attgagagaagtccaccacga

SEQ ID NO: 5 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 3', 4' seco modified nucleotide with phosphorothioate linkage
  modified_base 21: mod_base = OTHER; note = 2'-O-methyl modified nucleotide
attgagagaagtccaccacga

SEQ ID NO: 6 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = L-2'-O-Methyl modified nucleotide with phosphorothioate linkage
  modified_base 21: mod_base = OTHER; note = 2'-O-methyl modified nucleotide
attgagagaagtccaccacga

SEQ ID NO: 7 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 8 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = N1-stabilizing modified nucleotide
attgagagaagtccaccacga

SEQ ID NO: 9 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 3'-methyl modified nucleotide with phosphorothioate linkage
  modified_base 21: mod_base = OTHER; note = 2'-O-methyl modified nucleotide
attgagagaagtccaccacga

SEQ ID NO: 10 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 11 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ccgtgtgcacttcgcttca

SEQ ID NO: 12 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 13 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 14 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-cyclopropyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 15 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl-cyclopropyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 16 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-cyclopropyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 17 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl-cyclopropyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 18 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-cyclopropyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 19 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl-cyclopropyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 20 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 21 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 22 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-cyclopropyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 23 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl-cyclopropyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 24 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 25 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 26 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-cyclopropyl modified nucleotide
tgaagcgaagtgcacacggtc

SEQ ID NO: 27 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl-cyclopropyl modified nucleotide
tgaagcgaagtgcacacggtc

SEQ ID NO: 28 - RNA, 19 nt, synthetic construct
  source 1..19: mol_type = other RNA
  modified_base 1: mod_base = OTHER; note = 2'-O-cyclopropyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 29 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 30 - RNA, 19 nt, synthetic construct
  source 1..19: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 31 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-cyclopropyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 32 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl-cyclopropyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 33 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-cyclopropyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 34 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl-cyclopropyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 35 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-cyclopropyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 36 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl-cyclopropyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 37 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-cyclopropyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 38 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl-cyclopropyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 39 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-cyclopropyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 40 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl-cyclopropyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 41 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-cyclopropyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 42 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl-cyclopropyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 43 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-cyclopropyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 44 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl-cyclopropyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 45 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-cyclopropyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 46 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl-cyclopropyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 47 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-cyclopropyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 48 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl-cyclopropyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 49 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-cyclopropyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 50 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl-cyclopropyl modified nucleotide
  modified_base 18: mod_base = OTHER; note = 2'-O-methyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 51 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-cyclopropyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 52 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl-cyclopropyl modified nucleotide
  modified_base 19: mod_base = OTHER; note = 2'-O-methyl modified nucleotide attached to 4x N-acetylgalactosamine
ccgtgtgcacttcgcttca

SEQ ID NO: 53 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
ccgtgtgcacttcgcttca

SEQ ID NO: 54 - RNA, 19 nt, synthetic construct
  source 1..19: mol_type = other RNA
  modified_base 1: mod_base = OTHER; note = 2'-O-cyclopropyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-cyclopropyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-cyclopropyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-cyclopropyl modified nucleotide
  modified_base 11: mod_base = OTHER; note = 2'-O-methyl modified nucleotide
  modified_base 12: mod_base = OTHER; note = 2'-O-cyclopropyl modified nucleotide
  modified_base 13: mod_base = OTHER; note = 2'-O-methyl modified nucleotide
  modified_base 14: mod_base = OTHER; note = 2'-O-cyclopropyl modified nucleotide
  modified_base 15: mod_base = OTHER; note = 2'-O-methyl modified nucleotide
  modified_base 16: mod_base = OTHER; note = 2'-O-cyclopropyl modified nucleotide
  modified_base 17: mod_base = OTHER; note = 2'-O-methyl modified nucleotide
  modified_base 18: mod_base = OTHER; note = 2'-O-cyclopropyl modified nucleotide
  modified_base 19: mod_base = OTHER; note = 2'-O-methyl modified nucleotide with a phosphorothioate linkage attached to 4x N-acetylgalactosamine
ccgtgtgcacttcgcttca

SEQ ID NO: 55 - RNA, 19 nt, synthetic construct
  source 1..19: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl-cyclopropyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl-cyclopropyl modified nucleotide
  modified_base 11: mod_base = OTHER; note = 2'-O-methyl modified nucleotide
  modified_base 12: mod_base = OTHER; note = 2'-O-methyl-cyclopropyl modified nucleotide
  modified_base 13: mod_base = OTHER; note = 2'-O-methyl modified nucleotide
  modified_base 14: mod_base = OTHER; note = 2'-O-methyl-cyclopropyl modified nucleotide
  modified_base 15: mod_base = OTHER; note = 2'-O-methyl modified nucleotide
  modified_base 16: mod_base = OTHER; note = 2'-O-methyl-cyclopropyl modified nucleotide
  modified_base 17: mod_base = OTHER; note = 2'-O-methyl modified nucleotide
  modified_base 18: mod_base = OTHER; note = 2'-O-methyl-cyclopropyl modified nucleotide
  modified_base 19: mod_base = OTHER; note = 2'-O-methyl-cyclopropyl modified nucleotide with phosphorothioate linkage attached to 4x N-acetylgalactosamine
ccgtgtgcacttcgcttca

SEQ ID NO: 56 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
ccgtgtgcacttcgcttca

SEQ ID NO: 57 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-cyclopropyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-cyclopropyl modified nucleotide
  modified_base 11: mod_base = OTHER; note = 2'-O-methyl modified nucleotide
  modified_base 12: mod_base = OTHER; note = 2'-O-cyclopropyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-cyclopropyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 58 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-cyclopropyl modified nucleotide
  modified_base 11: mod_base = OTHER; note = 2'-O-methyl modified nucleotide
  modified_base 12: mod_base = OTHER; note = 2'-O-cyclopropyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-cyclopropyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 59 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl-cyclopropyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl-cyclopropyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl-cyclopropyl modified nucleotide
  modified_base 11: mod_base = OTHER; note = 2'-O-methyl modified nucleotide
  modified_base 12: mod_base = OTHER; note = 2'-O-methyl-cyclopropyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl-cyclopropyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 60 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl-cyclopropyl modified nucleotide
  modified_base 10: mod_base = OTHER; note = 2'-O-methyl-cyclopropyl modified nucleotide
  modified_base 11: mod_base = OTHER; note = 2'-O-methyl modified nucleotide
  modified_base 12: mod_base = OTHER; note = 2'-O-methyl-cyclopropyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl-cyclopropyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl modified nucleotide
tgaagcgaagtgcacacggtc

SEQ ID NO: 61 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-cyclopropyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-cyclopropyl modified nucleotide
tgaagcgaagtgcacacggtc

SEQ ID NO: 62 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-cyclopropyl modified nucleotide
  modified_base 4: mod_base = OTHER; note = 2'-O-cyclopropyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-cyclopropyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-cyclopropyl modified nucleotide
tgaagcgaagtgcacacggtc

SEQ ID NO: 63 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl-cyclopropyl modified nucleotide
tgaagcgaagtgcacacggtc

SEQ ID NO: 64 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl-cyclopropyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl-cyclopropyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl-cyclopropyl modified nucleotide
tgaagcgaagtgcacacggtc

SEQ ID NO: 65 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-methyl modified nucleotide
attgagagaagtccaccacga

SEQ ID NO: 66 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl modified nucleotide
attgagagaagtccaccacga

SEQ ID NO: 67 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-cyclopropyl modified nucleotide
attgagagaagtccaccacga

SEQ ID NO: 68 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl-cyclopropyl modified nucleotide
attgagagaagtccaccacga

SEQ ID NO: 69 - RNA, 19 nt, synthetic construct
  source 1..19: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-O-methyl-cyclopropyl modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl-cyclopropyl modified nucleotide
  modified_base 5: mod_base = OTHER; note = 2'-fluoro modified nucleotide
  modified_base 6: mod_base = OTHER; note = 2'-O-methyl-cyclopropyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl-cyclopropyl modified nucleotide
  modified_base 11: mod_base = OTHER; note = 2'-O-methyl-cyclopropyl modified nucleotide
  modified_base 12: mod_base = OTHER; note = 2'-O-methyl-cyclopropyl modified nucleotide
  modified_base 13: mod_base = OTHER; note = 2'-O-methyl-cyclopropyl modified nucleotide
  modified_base 14: mod_base = OTHER; note = 2'-O-methyl-cyclopropyl modified nucleotide
  modified_base 15: mod_base = OTHER; note = 2'-O-methyl-cyclopropyl modified nucleotide
  modified_base 16: mod_base = OTHER; note = 2'-O-methyl-cyclopropyl modified nucleotide
  modified_base 17: mod_base = OTHER; note = 2'-O-methyl-cyclopropyl modified nucleotide
  modified_base 18: mod_base = OTHER; note = 2'-O-methyl-cyclopropyl modified nucleotide
  modified_base 19: mod_base = OTHER; note = 2'-O-methyl-cyclopropyl modified nucleotide with phosphorothioate linkage attached to 4x N-acetylgalactosamine
ccgtgtgcacttcgcttca

SEQ ID NO: 70 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl-cyclopropyl modified nucleotide
  modified_base 5: mod_base = OTHER; note = 2'-fluoro modified nucleotide
  modified_base 6: mod_base = OTHER; note = 2'-O-methyl-cyclopropyl modified nucleotide
  modified_base 7: mod_base = OTHER; note = 2'-O-methyl-cyclopropyl modified nucleotide
  modified_base 8: mod_base = OTHER; note = 2'-fluoro modified nucleotide
  modified_base 9: mod_base = OTHER; note = 2'-O-methyl-cyclopropyl modified nucleotide
  modified_base 10: mod_base = OTHER; note = 2'-O-methyl-cyclopropyl modified nucleotide
  modified_base 11: mod_base = OTHER; note = 2'-O-methyl-cyclopropyl modified nucleotide
  modified_base 12: mod_base = OTHER; note = 2'-O-methyl-cyclopropyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-O-methyl-cyclopropyl modified nucleotide
  modified_base 17: mod_base = OTHER; note = 2'-fluoro modified nucleotide
  modified_base 18: mod_base = OTHER; note = 2'-O-methyl-cyclopropyl modified nucleotide
  modified_base 19: mod_base = OTHER; note = 2'-O-methyl-cyclopropyl modified nucleotide with phosphorothioate linkage
  modified_base 20: mod_base = OTHER; note = 2'-O-methyl-cyclopropyl modified nucleotide with phosphorothioate linkage
  modified_base 21: mod_base = OTHER; note = 2'-O-methyl-cyclopropyl modified nucleotide
tgaagcgaagtgcacacggtc

SEQ ID NO: 71 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = unlocked nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 72 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 3', 4' seco modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 73 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 74 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 3'-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 75 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 3-hydroxy modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 76 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = unlocked nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 77 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 3', 4' seco modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 78 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 79 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 80 - RNA, 21 nt, synthetic construct
  source 1..21: mol_type = other RNA
  modified_base 1: mod_base = OTHER; note = 5-(R)cyclopropyl 2-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 81 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 5-(R)cyclopropyl 2-O-methyl modified nucleotide
attgagagaagtccaccacga

SEQ ID NO: 82 - RNA, 21 nt, synthetic construct
  source 1..21: mol_type = other RNA
  modified_base 1: mod_base = OTHER; note = 5-(S)cyclopropyl 2-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 83 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 5-(S)cyclopropyl 2-O-methyl modified nucleotide
attgagagaagtccaccacga

SEQ ID NO: 84 - RNA, 21 nt, synthetic construct
  source 1..21: mol_type = other RNA
  modified_base 1: mod_base = OTHER; note = 5-(R)methylcyclopropyl 2-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 85 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 5-(R)methylcyclopropyl 2-O-methyl modified nucleotide
attgagagaagtccaccacga

SEQ ID NO: 86 - RNA, 21 nt, synthetic construct
  source 1..21: mol_type = other RNA
  modified_base 1: mod_base = OTHER; note = 5-(S)methylcyclopropyl 2-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 87 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 5-(S)methylcyclopropyl 2-O-methyl modified nucleotide
attgagagaagtccaccacga

SEQ ID NO: 88 - skipped

SEQ ID NO: 89 - DNA, 3182 bp, Hepatitis B virus
  source 1..3182: mol_type = genomic DNA
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 90 - DNA, 7459 bp, Homo sapiens
  source 1..7459: mol_type = genomic DNA
agtcactgccgcggcgccttgagtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggactgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgcttaaggaccacactgaaggaaaaaaaaagaggggacttcgaaaaaaaaaaaagccctgcaaaatattctaaaacatggtcttcttaattttctatatggattgaccacagtcttatcttccaccattaagctgtataacaataaaatgttaatagtcttgctttttattatcttttaaagatctccttaaattctataactgatcttttttcttattttgtttgtgacattcatacatttttaagatttttgttatgttctgaattcccccctacacacacacacacacacacacacacacacacgtgcaaaaaatatgatcaagaatgcaattgggatttgtgagcaatgagtagacctcttattgtttatatttgtaccctcattgtcaatttttttttagggaatttgggactctgcctatataaggtgttttaaatgtcttgagaacaagcactggctgatacctcttggagatatgatctgaaatgtaatggaatttattaaatggtgtttagtaaagtaggggttaaggacttgttaaagaaccccactatctctgagaccctatagccaaagcatgaggacttggagagctactaaaatgattcaggtttacaaaatgagccctgtgaggaaaggttgagagaagtctgaggagtttgtatttaattatagtcttccagtactgtatattcattcattactcattctacaaatatttattgaccccttttgatgtgcaaggcactatcgtgcgtcccctgagagttgcaagtatgaagcagtcatggatcatgaaccaaaggaacttatatgtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatctttcaacttctaacattctgtagtttaggagttctcaaccttggcattattgacatgttaggccaaataattttttttgtgggaggtctcttgtgcgttttagatgattagcaataatccctgacctgttatctactaaagactagtcgtttctcatcagttgtgacaacaaaaatggttccagatattgccaaatgccctttagaggacagtaatcgcccccagttgagaaccatttcagtaaaactttaattactattttttcttttggtttataaaataatgatcctgaattaaattgatggaaccttgaagtcgataaaatatatttcttgctttaaagtccccatacgtgtcctactaattttctcatgctttagtgttttcacttttctcctgttatccttgtacctaagaatgccatcccaatccccagatgtccacctgcccaaagtctaggcatagctgaaggccaagctaaaatgtatccctctttttctggtacatgcagcaaaagtaatatgaattatcagctttctgagagcaggcattgtatctgtcttgtttggtgttacattggcacccaataaatatttgttgagtgaatgaataaattcccatagcactttattcttcacatggtacataactataggggctatagcttggtaccttgtgaagcaactcttggtgtaacataccttatttctcatactaaaatgcaagaacctttagagcaaggatcttgccattcatctttgtaacctctttactctggagcacttgcatttagcaggcatcataaagttttacgtaccaagaaaatgttgctgttttctgaatactatgcatcaaaaaatgttaccactaatttttaaagctctgctaaggaatattggggcaccctcagatgcaccttttaattgatgtcatattttcctaatccatactttattcatgagaatttgagtcaccccagcattagcttggaatttccttatttcccatttgctttgcaggtgccttggagtcagatctggttttgaatactatcttcctgttatgtgatcttgggcagttacttaattttctagtcaataacccgtatctataaaatagagaaaataatcctacacaccggggcctgttgtggggcggggagaggggggagggatcgcatttggagatatactaatgtaaatgacaagttaattggtgcagcacaccaacatggctcatgtctacatatgtaacaaacctgcacgttgtgcacatgtgccctagaacttaaagtataataaaaagaaattttaaaaaatcctgtcaaataaggttatagtagagaataaggatgtgtaaagcatttagtcacgtaaatgcttaaaaaaatgtaatttttacttctttcactgcctcatttaattagttttatctttaataataccttggattcagggtaaagtttcagttatgtcccagtaatcatttattttaccctcgaatctgcaatttggatagaacatggtggggacagctcgtctctattccttgcagcattaacaggctggaggcaccacttctctggccagcaagttgggcctggttgttggctgagagcctcagttcctttctgcacaggttcctctttacataggcttctcaacagggctactagagcatcgtcaccatagcagctgtcttataacagagagtggtcggtctgagagacaaaaaatggaagctgccaaattgttctgggtctggaaactgtcagggcatcacttgtgccatattcagttggcctaagaattacagagcctgcctcgattcaaagggagaggatagagaggactgaaggaatcagtgctcatctttaatatgcagcaggacaggtttgggattttttttcccccttgagtctgtgaaggcattacttaagaacaaagtcaggcatgtataattgaactacagttacttgaaatataagcccagaaagtttcagataataaatacaactatttttctgctgttacccttgtacctaaagatgccatcctaatccccagatctccacaactatacctacatagtagaaggttaaaatgtatccctctttttctggtgcatccagcaaaagtaatatcatgaattatgagctctctgagagcaaggatcatatcagtcttgtttattgttgcagtgaacaagtacagttgcagatattcaggagtaattatctaaatggcagtaggcttataaaactgaattttcaccagccacaccctccccccaactccttatctgtaaaaagcttatttgagtggttacctgtcttcagtaaagattgcgcttgcatatttgctgtcattgcatattctgcttaattaagctctgttgatattgcagtttctgtgcatacttacatcttagatgcaatctgagggcctaggaaggccttttaaaaataaaacaattccgattgcagagaaagtgtaagtcaaggacagttaattcaaggggaacatagaaagctatttagattttagttgatggtgccagtcttcagcgtaaagtcaaaagtggagggaagtttagtaaggaaaaaatgttgggcttggaatacattgtttagtcttcaaagcactttactttttatgaaatatattttagacattcagcaaatattgaatacttactatatcaggcagtaaagatataaattcattcttaaaatgtgcaacatgttcaaactgaaaaaaatacattcttaaacaggaaactttttccttcatactttttaattaacaagacatataagagttgcattaatgggcgtgcttatgattgatcacccagcagcatcattagaaataatatattttattcatgtgcagaaatcttttggttgtcctggggaaccttgaacacagaaaagagcttttattgataaggtaattgaacacacttgacaattagcttaatatggtttaataccatttgtgggagaagatgaatcagccaggctctttacgtcaagaatatgaagtttctcttgagtcaaccaacttaagatgagctacggagactgcagtgaaaagttaaatatccaagtacaccagccaatttcacacagtggaaccatgctgtcctcgggcaccctgcacctcgcccaacagtcatcaactagatggaggctcctggctgcaaggaggatttgatgggaatgagtaaatgtgtcagcatagtccgtcccttctaatggaaaagcaacccaaagagcaaatcctattaatggctggatcagtatcatctacttgtcaaaaacattccatgaattatgagtcaaaattttatttatggtggcattacacacattaagagatgaggacttctgttagcataatttattagctggaaaagttgagaaggttctctggactcatttttataggtggaacctaagtgatctggataattgcccaccagcaaaattgctgggcatggtggacaaagaaaatgttccttctaatgattttttatgagctgagtagctattgttcccagctgagtgctcttttcctctttttattgttgctgagcaaaagaatttataaaaagctctttcttttgtattaaaaaccctgctcaattgaaatgcaagttcattaagtaatcttcatttctcttcctgccataataaccctttccctctctgttcgattcaacagtatctagcagcactgctccaaattttaagtctgaacagactatattacatagatgtagagaaatactcaatcttcagcattaagagggagcttaatttcacacgggtggaatatgatcactcaggctagatgttggccataaatttcaaattagtatctcaacttagcaggggggatcaacagtggcaaacttcaattatgacaggataaaaatcacatagagatattggttcaatatggacatctaaactataatgctaaaagccaataattagaataagttcattttaagaaaagcattaataatattagctaacgtttagtacctgtgccaaacattctacctatgttaccttgattttcatagccagcctaagaggtactattatgtatccccattttacaggttaagaaacaggctcagaggagtttaggatcttttccaagattacatagccagtaagtggtggcactaggaaccaaattcagactctgaatcgcatgctgtttatattatattgcactcattctaaatatgtgggaatcagaatgaaggggcttgtatgacttttggctcattttttgatgcatgtgacctgggattataaatgtgaaattaggtttacgaaaggatccagtgtcattgtgcatcatgggcaaggagtacctaatctctttaattcttccctggaagcttacgatgtccatccaagtgcacatagcaaaagttctgttgtaaagtttagcagagtgactttctttgactcagagtgatgacggaggaagctttgataagattttatctgaaatgttcatggacaagagctttcaaggagaacatccagagcaaggttctgaagacagctcatgaaggtgaagcagcagacctggcacaagaaatgaagagagagctcagtgtattaaagatgaaaacaagaaaaccgaatatattgaaaggagcagagaggcaatgaaaacaagacaactgaaatgaggtaacttgcagcaattgaaagggaatttcagtacttttatagaattcttaaaaattgtttcctgctgtttattttcaattttgaacagggttatttgtccatgccatactttttttgccaaattccaaaattgtgtatagttctatagttgtctggtggagtcaatggaactttagttaccagtctaagaatgtgtctttgagattgtccagttaattctctatttccagtagctgtaataaatggtgaaaaggtttctgactcctggagaaagtttctaactccttatgactaatattcataacagacttgtgagttccttgaacatggatacacctatatgcaagagtgtattccaaagctaactcagtgatctttccatttatctattcttggattagtggtgcctttgctctttccttctgtaaatgtgaatagttaagagttgactgcagaagtgtttacactttggcttccatgcctctggaatgtttgtgctttggtggtgagatgtgagactatatttgtatagtctgcatctctcaggctgccccagaatgttgtacagtgcagtgctgaagaaagcagcaggtacacacagaaatgcagcctttcctggttaaccctgcttggatctgagttacactttgtttcctgacttcttgggacttaggtaatcagtttgccttctactctatctcattttgtactcgcttacatactacattcttgtttgggctttcgtttcttcttgtaagcagagattttttaaaatccaatatgtgaaaatacggatgcactacaattaaataaataaaatgctgttgtgtttgttttgctttaaaattgtaaaggataaacaataagatagttttatctatgtggttttcccgatgcagttaaaataaaacctaatctgctaaaattgaa

SEQ ID NO: 91 - DNA, 1906 bp, Homo sapiens
  source 1..1906: mol_type = genomic DNA
gctttccggcggttgcaccgggccggggtgccagcgcccgccttcccgtttcctcccgttccgcagcgcgcccacggcctgtgaccccggcgaccgctccccagtgacgagagagcggggccgggcgctgctccggcctgacctgcgaagggacctcggtccagtcccctgttgcgccgcgcccccgtccgtccgtgcgcgggccagtcaggggccagtgtctcgagcggtcgaggtcgcagacctagaggcgccccacaggccggcccggggcgctgggagcgccggccgcgggccgggtggggatgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagcagaatttttccaagcagagaatgaggggaaaggtgttctagacacaggcaggcacatgccaggtgctggaaaaagaagagagaaaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagataggccttgcttgctgccttctggattcttggcccgcacagagctggggctgagggatggactgatgcttttagctcaaacgtggcttttagacagatttgttcatagaccctctcaagtgccctctccgagctggtaggcattccagctcctccgtgcttcctcagttacacaaaggacctcagctgcttctcccacttggccaagcagggaggaagaagcttaggcagggctctctttccttcttgccttcagatgttctctcccaggggctggcttcaggagggagcatagaaggcaggtgagcaaccagttggctaggggagcagggggcccaccagagctgtggagaggggaccctaagactcctcggcctggctcctacccaccgcccttgccgaaccaggagctgctcactacctcctcagggatggccgttggccacgtcttccttctgcctgagcttcccccccaccacaggccctttcctcaggcaaggtctggcctcaggtgggccgcaggcgggaaaagcagcccttggccagaagtcaagcccagccacgtggagcctagagtgagggcctgaggtctggctgcttgcccccatgctggcgccaacaacttctccatcctttctgcctctcaacatcacttgaatcctagggcctgggttttcatgtttttgaaacagaaccataaagcatatgtgttggcttgttgtaaaa

SEQ ID NO: 92 - DNA, 2926 bp, Homo sapiens
  source 1..2926: mol_type = genomic DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 93 - DNA, 2336 bp, Homo sapiens
  source 1..2336: mol_type = genomic DNA
tgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtctgtgggttatttaaaagaaattataacaattttgctaaaccattacaatgttaggtcttttttaagaaggaaaaagtcagtatttcaagttctttcacttccagcttgccctgttctaggtggtggctaaatctgggcctaatctgggtggctcagctaacctctcttcttcccttcctgaagtgacaaaggaaactcagtcttcttggggaagaaggattgccattagtgacttggaccagttagatgattcactttttgcccctagggatgagaggcgaaagccacttctcatacaagcccctttattgccactaccccacgctcgtctagtcctgaaactgcaggaccagtttctctgccaaggggaggagttggagagcacagttgccccgttgtgtgagggcagtagtaggcatctggaatgctccagtttgatctcccttctgccacccctacctcacccctagtcactcatatcggagcctggactggcctccaggatgaggatgggggtggcaatgacaccctgcaggggaaaggactgccccccatgcaccattgcagggaggatgccgccaccatgagctaggtggagtaactggtttttcttgggtggctgatgacatggatgcagcacagactcagccttggcctggagcacatgcttactggtggcctcagtttaccttccccagatcctagattctggatgtgaggaagagatccctcttcagaaggggcctggccttctgagcagcagattagttccaaagcaggtggcccccgaacccaagcctcacttttctgtgccttcctgagggggttgggccggggaggaaacccaaccctctcctgtgtgttctgttatctcttgatgagatcattgcaccatgtcagacttttgtatatgccttgaaaataaatgaaagtgagaatcctctaaaaaaaaaaaa

SEQ ID NO: 94 - DNA, 3215 bp, Hepatitis B virus
  source 1..3215: mol_type = genomic DNA
ctccaccactttccaccaaactcttcaagatcccagagtcagggccctgtactttcctgctggtggctcaagttccggaacagtaaaccctgctccgactactgcctctcccatatcgtcaatcttctcgaggactggggaccctgtaccgaatatggagagcaccacatcaggattcctaggacccctgctcgtgttacaggcggggtttttcttgttgacaagaatcctcacaataccacagagtctagactcgtggtggacttctctcaattttctagggggagcacccacgtgtcctggccaaaatttgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactaccaaggtatgttgcccgtttgtcctctacttccaggaacatcaactaccagcaccggaccatgcaaaacctgcacaactactgctcaagggacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttataccgctgttaccaattttcttttatctttgggtatacatttaaaccctcacaaaacaaaaagatggggatattcccttaacttcatgggatatgtaattgggagttggggcactttgcctcaggaacatattgtacaaaaaatcaagcaatgttttaggaaacttcctgtaaacaggcctattgattggaaagtatgtcaacraattgtgggtcttttggggtttgccgcccctttcacgcaatgtggatatcctgctttaatgcctttatatgcatgtatacaagctaagcaggcttttactttctcgccaacttacaaggcctttctgtgtaaacaatatctgaacctttaccccgttgctcggcaacggtcaggtctttgccaagtgtttgctgacgcaacccccactggttggggcttggccataggccatcagcgcatgcgtggaacctttgtggctcctctgccgatccatactgcggaactcctagcagcttgttttgctcgcagccggtctggagcaaaacttatcggcaccgacaactctgttgtcctctctcggaaatacacctcctttccatggctgctaggatgtgctgccaactggatcctgcgcgggacgtcctttgtctacgtcccgtcggcgctgaatcccgcggacgacccatctcggggccgtttgggactctaccgtccccttctgcgtctgccgttccgcccgaccacggggcgcacctctctttacgcggtctccccgtctgtgccttctcatctgccggaccgtgtgcacttcgcttcacctctgcacgtcgcatggagaccaccgtgaacgcccacgggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagcaattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccacaaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagacttaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccacacaaggacaactggccggacgccaacaaggtgggagtgggagcattcgggccagggttcacccctcctcatgggggactgttggggtggagccctcaggctcagggcatattcacaacagtgccagcagctcctcctcctgcctccaccaatcggcagtcaggaaggcagcctactcccttctctccacctctaagagacactcatcctcaggccatgcagtggaa

SEQ ID NO: 95 - RNA, 18 nt, synthetic construct
  source 1..18: mol_type = other RNA
  modified_base 1: mod_base = OTHER; note = 4x N-acetylgalactosamine phosphorothioate 4x N-acetylgalactosamine phosphorothioate 4x N-acetylgalactosamine attached to 2'-O-methyl modified nucleotide
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Locked thymine with phosphorothioate linkage
  modified_base 5: mod_base = OTHER; note = Locked nucleic acid with phosphorothioate linkage
  modified_base 6: mod_base = OTHER; note = Locked nucleic acid with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-hydroxy modified nucleotide with phosphorothioate linkage
  modified_base 10: mod_base = OTHER; note = Nucleotide with phosphorothioate linkage
  modified_base 11: mod_base = OTHER; note = 5-methyl modified nucleotide with phosphorothioate linkage
  modified_base 12: mod_base = OTHER; note = 5-methyl modified nucleotide with phosphorothioate linkage
  modified_base 13: mod_base = OTHER; note = Nucleotide with phosphorothioate linkage
  modified_base 14: mod_base = OTHER; note = 5-methyl modified nucleotide with phosphorothioate linkage
  modified_base 15: mod_base = OTHER; note = Locked nucleic acid with phosphorothioate linkage
  modified_base 16: mod_base = OTHER; note = Locked nucleic acid with phosphorothioate linkage
  modified_base 17: mod_base = OTHER; note = Locked nucleic acid with phosphorothioate linkage
  modified_base 18: mod_base = OTHER; note = Spirocyclopropane crosslinked 5-methyl modified nucleotide
agataaaacgccgcagac

SEQ ID NO: 96 - RNA, 18 nt, synthetic construct
  source 1..18: mol_type = other RNA
  modified_base 1: mod_base = OTHER; note = 2'-O-methyl modified nucleotide
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Locked thymine with phosphorothioate linkage
  modified_base 5: mod_base = OTHER; note = Locked nucleic acid with phosphorothioate linkage
  modified_base 6: mod_base = OTHER; note = Locked nucleic acid with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-hydroxy modified nucleotide with phosphorothioate linkage
  modified_base 10: mod_base = OTHER; note = Nucleotide with phosphorothioate linkage
  modified_base 11: mod_base = OTHER; note = 5-methyl modified nucleotide with phosphorothioate linkage
  modified_base 12: mod_base = OTHER; note = 5-methyl modified nucleotide with phosphorothioate linkage
  modified_base 13: mod_base = OTHER; note = Nucleotide with phosphorothioate linkage
  modified_base 14: mod_base = OTHER; note = 5-methyl modified nucleotide with phosphorothioate linkage
  modified_base 15: mod_base = OTHER; note = Locked nucleic acid with phosphorothioate linkage
  modified_base 16: mod_base = OTHER; note = Locked nucleic acid with phosphorothioate linkage
  modified_base 17: mod_base = OTHER; note = Locked nucleic acid with phosphorothioate linkage
  modified_base 18: mod_base = OTHER; note = Spirocyclopropane crosslinked 5-methyl modified nucleotide
agataaaacgccgcagac

SEQ ID NO: 97 - DNA, 29903 bp, Severe acute respiratory syndrome coronavirus 2
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 98 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro-3'-xylo modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 99 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro-3'-xylo modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 100 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro-3'-xylo modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 101 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro-3'-xylo modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 102 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro-3'-xylo modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 103 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro-3'-xylo modified nucleotide
  modified_base 18: mod_base = OTHER; note = 2'-O-methyl modified nucleotide
  modified_base 19: mod_base = OTHER; note = 2'-O-methyl modified nucleotide with a phosphorothioate linkage attached to 4x N-acetylgalactosamine
gtggtggacttctctcaat

SEQ ID NO: 104 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro-3'-xylo modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 105 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro-3'-xylo modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 106 - RNA, 19 nt, synthetic construct
  source 1..19: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gtgtgcacttcgcttcaca

SEQ ID NO: 107 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = Glycol nucleic acid
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgtgaagcgaagtgcacactt

SEQ ID NO: 108 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 109 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 110 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 111 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 112 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 113 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 114 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 115 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-fluoro modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 116 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = ganciclovir-(R)guanosine
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 117 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = ganciclovir-(S)guanosine
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 118 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = denvir-(R)guanosine
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 119 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = denvir-(S)guanosine
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 120 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = ganciclovir-(R)guanosine
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 121 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = ganciclovir-(S)guanosine
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 122 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = denvir-(R)guanosine
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 123 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = denvir-(S)guanosine
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 124 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 3'- O-cyclopropyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 125 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 3'- O-cyclopropyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 126 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-Methyl-3'-Xylo modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 127 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-Methyl-3'-Xylo modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 128 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-Methyl-3'-Xylo modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 129 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-Methyl-3'-Xylo modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 130 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-Methyl-3'-Xylo modified nucleotide with phosphorothioate linkage
  modified_base 21: mod_base = OTHER; note = 2'-O-methyl modified nucleotide
attgagagaagtccaccacga

SEQ ID NO: 131 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-Methyl-3'-Xylo modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-Methyl-3'-Xylo modified nucleotide with phosphorothioate linkage
  modified_base 21: mod_base = OTHER; note = 2'-O-methyl modified nucleotide
attgagagaagtccaccacga

SEQ ID NO: 132 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-Methyl-3'-Xylo modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-Methyl-3'-Xylo modified nucleotide with phosphorothioate linkage
  modified_base 21: mod_base = OTHER; note = 2'-O-methyl modified nucleotide
attgagagaagtccaccacga

SEQ ID NO: 133 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-Methyl-3'-Xylo modified nucleotide
attgagagaagtccaccacga

SEQ ID NO: 134 - RNA, 21 nt, synthetic construct
  source 1..21: mol_type = other RNA
  modified_base 1: mod_base = OTHER; note = 2'-O-Methyl-3'-Xylo modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 135 - RNA, 21 nt, synthetic construct
  source 1..21: mol_type = other RNA
  modified_base 1: mod_base = OTHER; note = 2'-O-Methyl-3'-Xylo modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 136 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-Methyl-3'-Xylo modified nucleotide with phosphorothioate linkage
  modified_base 21: mod_base = OTHER; note = 2'-O-methyl modified nucleotide
attgagagaagtccaccacga

SEQ ID NO: 137 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
attgagagaagtccaccacga

SEQ ID NO: 138 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 139 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl-cyclopropyl modified nucleotide
attgagagaagtccaccacga

SEQ ID NO: 140 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 141 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
attgagagaagtccaccacga

SEQ ID NO: 142 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl-cyclopropyl modified nucleotide
attgagagaagtccaccacga

SEQ ID NO: 143 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-cyclopropyl modified nucleotide with phosphorothioate linkage
  modified_base 21: mod_base = OTHER; note = 2'-O-cyclopropyl modified nucleotide
attgagagaagtccaccacga

SEQ ID NO: 144 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-cyclopropyl modified nucleotide
  modified_base 10: mod_base = OTHER; note = 2'-O-methyl modified nucleotide
  modified_base 11: mod_base = OTHER; note = 2'-O-cyclopropyl modified nucleotide
  modified_base 12: mod_base = OTHER; note = 2'-O-methyl modified nucleotide
  modified_base 13: mod_base = OTHER; note = 2'-O-cyclopropyl modified nucleotide
  modified_base 14: mod_base = OTHER; note = 2'-fluoro modified nucleotide
  modified_base 15: mod_base = OTHER; note = 2'-O-cyclopropyl modified nucleotide
  modified_base 16: mod_base = OTHER; note = 2'-fluoro modified nucleotide
  modified_base 17: mod_base = OTHER; note = 2'-O-cyclopropyl modified nucleotide
  modified_base 18: mod_base = OTHER; note = 2'-O-methyl modified nucleotide
  modified_base 19: mod_base = OTHER; note = 2'-O-cyclopropyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
attgagagaagtccaccacga

SEQ ID NO: 145 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl-cyclopropyl modified nucleotide
  modified_base 10: mod_base = OTHER; note = 2'-O-methyl modified nucleotide
  modified_base 11: mod_base = OTHER; note = 2'-O-methyl-cyclopropyl modified nucleotide
  modified_base 12: mod_base = OTHER; note = 2'-O-methyl modified nucleotide
  modified_base 13: mod_base = OTHER; note = 2'-O-methyl-cyclopropyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-fluoro modified nucleotide
  modified_base 17: mod_base = OTHER; note = 2'-O-methyl-cyclopropyl modified nucleotide
  modified_base 18: mod_base = OTHER; note = 2'-O-methyl modified nucleotide
  modified_base 19: mod_base = OTHER; note = 2'-O-methyl-cyclopropyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl-cyclopropyl modified nucleotide
attgagagaagtccaccacga

SEQ ID NO: 146 - RNA, 21 nt, synthetic construct
  source 1..21: mol_type = other RNA
  modified_base 1: mod_base = OTHER; note = 2'-O-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-cyclopropyl modified nucleotide
  modified_base 4: mod_base = OTHER; note = 2'-O-methyl modified nucleotide
  modified_base 5: mod_base = OTHER; note = 2'-O-cyclopropyl modified nucleotide
  modified_base 6: mod_base = OTHER; note = 2'-fluoro modified nucleotide
  modified_base 7: mod_base = OTHER; note = 2'-O-cyclopropyl modified nucleotide
  modified_base 8: mod_base = OTHER; note = 2'-O-methyl modified nucleotide
  modified_base 9: mod_base = OTHER; note = 2'-O-cyclopropyl modified nucleotide
  modified_base 10: mod_base = OTHER; note = 2'-O-methyl modified nucleotide
  modified_base 11: mod_base = OTHER; note = 2'-O-cyclopropyl modified nucleotide
  modified_base 12: mod_base = OTHER; note = 2'-O-methyl modified nucleotide
  modified_base 13: mod_base = OTHER; note = 2'-O-cyclopropyl modified nucleotide
  modified_base 14: mod_base = OTHER; note = 2'-fluoro modified nucleotide
  modified_base 15: mod_base = OTHER; note = 2'-O-cyclopropyl modified nucleotide
  modified_base 16: mod_base = OTHER; note = 2'-fluoro modified nucleotide
  modified_base 17: mod_base = OTHER; note = 2'-O-cyclopropyl modified nucleotide
  modified_base 18: mod_base = OTHER; note = 2'-O-methyl modified nucleotide
  modified_base 19: mod_base = OTHER; note = 2'-O-cyclopropyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
attgagagaagtccaccacga

SEQ ID NO: 147 - RNA, 21 nt, synthetic construct
  source 1..21: mol_type = other RNA
  modified_base 1: mod_base = OTHER; note = 2'-O-methyl-cyclopropyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl-cyclopropyl modified nucleotide
  modified_base 4: mod_base = OTHER; note = 2'-O-methyl modified nucleotide
  modified_base 5: mod_base = OTHER; note = 2'-O-methyl-cyclopropyl modified nucleotide
  modified_base 6: mod_base = OTHER; note = 2'-fluoro modified nucleotide
  modified_base 7: mod_base = OTHER; note = 2'-O-methyl-cyclopropyl modified nucleotide
  modified_base 8: mod_base = OTHER; note = 2'-O-methyl modified nucleotide
  modified_base 9: mod_base = OTHER; note = 2'-O-methyl-cyclopropyl modified nucleotide
  modified_base 10: mod_base = OTHER; note = 2'-O-methyl modified nucleotide
  modified_base 11: mod_base = OTHER; note = 2'-O-methyl-cyclopropyl modified nucleotide
  modified_base 12: mod_base = OTHER; note = 2'-O-methyl modified nucleotide
  modified_base 13: mod_base = OTHER; note = 2'-O-methyl-cyclopropyl modified nucleotide
  modified_base 14: mod_base = OTHER; note = 2'-fluoro modified nucleotide
  modified_base 15: mod_base = OTHER; note = 2'-O-methyl-cyclopropyl modified nucleotide
  modified_base 16: mod_base = OTHER; note = 2'-fluoro modified nucleotide
  modified_base 17: mod_base = OTHER; note = 2'-O-methyl-cyclopropyl modified nucleotide
  modified_base 18: mod_base = OTHER; note = 2'-O-methyl modified nucleotide
  modified_base 19: mod_base = OTHER; note = 2'-O-methyl-cyclopropyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl-cyclopropyl modified nucleotide
attgagagaagtccaccacga

SEQ ID NO: 148 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 149 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
tgaagcgaagtgcacacggtc

SEQ ID NO: 150 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl-cyclopropyl modified nucleotide
tgaagcgaagtgcacacggtc

SEQ ID NO: 151 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-cyclopropyl modified nucleotide
attgagagaagtccaccacga

SEQ ID NO: 152 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
agaagatgaggcatagcagtt

SEQ ID NO: 153 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = denvir-(S)guanosine
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 154 - RNA, 21 nt, synthetic construct
  source 1..21: mol_type = other RNA
  modified_base 1: mod_base = OTHER; note = 5'-vinyl phosphonate 2'-O-methyl nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 1', 2' seco modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 155 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 156 - RNA, 21 nt, synthetic construct
  source 1..21: mol_type = other RNA
  misc_difference 1: note = Threose modified adenosine, cytidine, uridine, guanosine, unknown or other nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
ngaagcgaagtgcacacggtc

SEQ ID NO: 157-160 - skipped (4 entries)

SEQ ID NO: 161 - RNA, 21 nt, synthetic construct
  source 1..21: mol_type = other RNA
  modified_base 1: mod_base = OTHER; note = 5'-vinyl phosphonate 2'-O-methyl nucleotide with R-stereo-defined phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 162 - RNA, 21 nt, synthetic construct
  source 1..21: mol_type = other RNA
  modified_base 1: mod_base = OTHER; note = 5'-vinyl phosphonate 2'-O-methyl nucleotide with S-stereo-defined phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgaagcgaagtgcacacggtc

SEQ ID NO: 163 - RNA, 21 nt, synthetic construct
  source 1..21: mol_type = other RNA
  modified_base 1: mod_base = OTHER; note = deuterated 5' vinyl phosphonate nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 164 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga

SEQ ID NO: 165 - RNA, 19 nt, synthetic construct
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ctgctatgcctcatcttct

SEQ ID NO: 166 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro-3'-xylo modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
attgagagaagtccaccacga